(19) United States
(12) Patent Application Publication
(10) Pub. No.: US 2026/0035335 A1
(43) Pub. Date: Feb. 5, 2026

(54) PI3K-ALPHA INHIBITORS AND METHODS OF USE THEREOF

(71) Applicant: Relay Therapeutics, Inc., Cambridge, MA (US)

(72) Inventors: Yue Pan, Cambridge, MA (US); Alessandro Boezio, Cambridge, MA (US); Cary Griffin Fridrich, Cambridge, MA (US); Elaine B. Krueger, Cambridge, MA (US); Demetri T. Moustakas, Cambridge, MA (US); Kevin David Raynor, Cambridge, MA (US); Kelley C. Shortsleeves, Cambridge, MA (US); Thomas H. McLean, Cambridge, MA (US); Michael Paul DeNinno, Gales Ferry, CT (US); Christopher Thomson, Hoddesdon (GB); Alexandre Larivee, Montreal (CA); Andrew J. Burnie, Montreal (CA); Bren-Jordan Atienza, Montreal (CA); Christian Perreault, Montreal (CA); Gaetan Maertens, Montreal (CA); Tarek Mohamed, Montreal (CA); Thomas Lepitre, Montreal (CA); Erich W. Baum, Cambridge, MA (US); Hakan Gunaydin, Cambridge, MA (US)

(21) Appl. No.: 19/296,535

(22) Filed: Aug. 11, 2025

Related U.S. Application Data
(63) Continuation of application No. 18/863,452.
(60) Provisional application No. 63/364,459, filed on May 10, 2022.

(51) Int. Cl. C07C 211/29; A61K 31/137; A61K 31/325; A61K 31/403; A61K 31/4184; A61K 31/423; A61P 35/00; C07C 271/06; C07D 209/54; C07D 235/02; C07D 263/52

(52) U.S. Cl. CPC C07C 211/29; A61K 31/137; A61K 31/325; A61K 31/403; A61K 31/4184; A61K 31/423; A61P 35/00; C07C 271/06; C07D 209/54; C07D 235/02; C07D 263/52

(57) ABSTRACT

The present disclosure relates to novel compounds and pharmaceutical compositions thereof, and methods for inhibiting the activity of PI3Kα enzymes with the compounds and compositions of the disclosure. The present disclosure further relates to, but is not limited to, methods for treating disorders associated with PI3Kα signaling with the compounds and compositions of the disclosure.

### CROSS-REFERENCE TO RELATED APPLICATIONS

This application claims the benefit of U.S. Provisional Application No. 63/364,459, filed on May 10, 2022, the entirety of which is hereby incorporated by reference.

### BACKGROUND

Phosphatidylinositol 3-kinases (PI3Ks) comprise a family of lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃), which, in turn, act as second messengers in signaling cascades by docking proteins containing pleckstrin-homology, FYVE, Phox and other phospholipid-binding domains into a variety of signaling complexes often at the plasma membrane (Vanhaesebroeck et al., Annu. Rev. Biochem 70:535 (2001); Katso et al., Annu. Rev. Cell Dev. Biol. 17:615 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens et al., Cell 89:105 (1997); Katso et al., Annu. Rev. Cell Dev. Biol. 17:615-675 (2001)).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). In many cases, PIP₂ and PIP₃ recruit Aid, the product of the human homologue of the viral oncogene v-Akt, to the plasma membrane where it acts as a nodal point for many intracellular signaling pathways important for growth and survival (Fantl et al., Cell 69:413-423 (1992); Bader et al., Nature Rev. Cancer 5:921 (2005); Vivanco and Sawyer, Nature Rev. Cancer 2:489 (2002)).

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

In view of the above, inhibitors of PI3Kα would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the clinical development of existing PI3K inhibitors has been the inability to achieve the required level of target inhibition in tumors while avoiding toxicity in cancer patients. Pan-PI3K inhibitors share certain target-related toxicities including diarrhea, rash, fatigue, and hyperglycemia. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (Hanker et al., Cancer Discovery (2019) PMID: 30837161. Therefore, selective inhibitors of PI3Kα may increase the therapeutic window, enabling sufficient target inhibition in the tumor while avoiding dose-limiting toxicity in cancer patients.

### SUMMARY

In some embodiments, the present disclosure provides a compound of formula I:

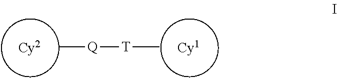

or a pharmaceutically acceptable salt thereof, wherein each of Cy¹, Cy², Q, and T is as defined in embodiments and classes and subclasses herein.

In some embodiments, the present disclosure provides a pharmaceutical composition comprising a compound of formula I, or a pharmaceutically acceptable salt thereof, and a pharmaceutically acceptable carrier, adjuvant, or diluent.

In some embodiments, the present disclosure provides a method of treating a PI3Kα-mediated disorder comprising administering to a patient in need thereof a compound of formula I, or composition comprising said compound.

In some embodiments, the present disclosure provides a process for providing a compound of formula I, or synthetic intermediates thereof.

In some embodiments, the present disclosure provides a process for providing pharmaceutical compositions comprising compounds of formula I.

### DETAILED DESCRIPTION

### 1. General Description of Certain Embodiments of the Disclosure

Compounds of the present disclosure, and pharmaceutical compositions thereof, are useful as inhibitors of PI3Kα. In some embodiments, the present disclosure provides a compound of formula I:

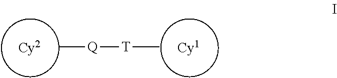

or a pharmaceutically acceptable salt thereof, wherein:
Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹;
Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R²;
Q is L^{Q};
T is a bivalent C₁₋₃ aliphatic chain substituted with q instances of R^{T};
each R¹ is independently -L¹-R^{1A};
each R² is independently -L²-R^{2A};
each R^{T} is independently -L^{T}-R^{TA}; or
   two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC};
   two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C};
   two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C};
   one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; or
   one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC};
each of L¹, L², L^{Q}, and L^{T} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R^{1A} is independently RA or R^{B} substituted by r¹ instances of R^{1C};
each R^{2A} is independently RA or R^{B} substituted by r² instances of R^{2C};
each RTA is independently RA or R^{B} substituted by r³ instances of R^{TC};
each R^{L} is independently RA or R^{B} substituted by r⁴ instances of R^{LC};
each instance of RA is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{TC}, R^{TTC}, R^{11C}, R^{22C}, R^{T1C}, R^{TLC}, and R^{LC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, m, q, p¹, p², p³, p⁴, p⁵, r¹, r², r³, and r⁴ is independently 0, 1, 2, 3, 4, or 5.

### 2. Compounds and Definitions

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl”, unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “C₁₋₈ (or C₁₋₆, or C₁₋₄) bivalent saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl,” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but is not limited to, phenyl, biphenyl, naphthyl, anthracyl and the like, which may bear one or more substituents.

The terms “heteroaryl” or “heteroaromatic”, unless otherwise defined, as used herein refers to a monocyclic aromatic 5-6 membered ring containing one or more heteroatoms, for example one to three heteroatoms, such as nitrogen, oxygen, and sulfur, or an 8-10 membered polycyclic ring system containing one or more heteroatoms, wherein at least one ring in the polycyclic ring system is aromatic, and the point of attachment of the polycyclic ring system is through a ring atom on an aromatic ring. A heteroaryl ring may be linked to adjacent radicals though carbon or nitrogen. Examples of heteroaryl rings include but are not limited to furan, thiophene, pyrrole, thiazole, oxazole, isothiazole, isoxazole, imidazole, pyrazole, triazole, pyridine, pyrimidine, indole, etc. For example, unless otherwise defined, 1,2,3,4-tetrahydroquinoline is a heteroaryl ring if its point of attachment is through the benzo ring, e.g.:

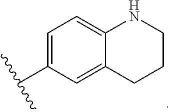 [SMILES: *c1ccc2c(c1)CCCN2]

The terms “heterocyclyl” or “heterocyclic group”, unless otherwise defined, refer to a saturated or partially unsaturated 3-10 membered monocyclic or 7-14 membered polycyclic ring system, including bridged or fused rings, and whose ring system includes one to four heteroatoms, such as nitrogen, oxygen, and sulfur. A heterocyclyl ring may be linked to adjacent radicals through carbon or nitrogen.

The term “partially unsaturated” in the context of rings, unless otherwise defined, refers to a monocyclic ring, or a component ring within a polycyclic (e.g., bicyclic, tricyclic, etc.) ring system, wherein the component ring contains at least one degree of unsaturation in addition to those provided by the ring itself, but is not aromatic. Examples of partially unsaturated rings include, but are not limited to, 3,4-dihydro-2H-pyran, 3-pyrroline, 2-thiazoline, etc. Where a partially unsaturated ring is part of a polycyclic ring system, the other component rings in the polycyclic ring system may be saturated, partially unsaturated, or aromatic, but the point of attachment of the polycyclic ring system is on a partially unsaturated component ring. For example, unless otherwise defined, 1,2,3,4-tetrahydroquinoline is a partially unsaturated ring if its point of attachment is through the piperidino ring, e.g.:

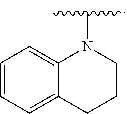 [SMILES: *N1CCCc2ccccc21]

The term “saturated” in the context of rings, unless otherwise defined, refers to a 3-10 membered monocyclic ring, or a 7-14 membered polycyclic (e.g., bicyclic, tricyclic, etc.) ring system, wherein the monocyclic ring or the component ring that is the point of attachment for the polycyclic ring system contains no additional degrees of unsaturation in addition to that provided by the ring itself. Examples of monocyclic saturated rings include, but are not limited to, azetidine, oxetane, cyclohexane, etc. Where a saturated ring is part of a polycyclic ring system, the other component rings in the polycyclic ring system may be saturated, partially unsaturated, or aromatic, but the point of attachment of the polycyclic ring system is on a saturated component ring. For example, unless otherwise defined, 2-azaspiro[3.4]oct-6-ene is a saturated ring if its point of attachment is through the azetidino ring, e.g.:

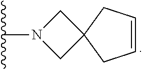 [SMILES: *N1CC2(CC=CC2)C1]

The terms “alkylene”, “arylene”, “cycloalkylene”, “heteroarylene”, “heterocycloalkylene”, and the other similar terms with the suffix “-ylene” as used herein refers to a divalently bonded version of the group that the suffix modifies. For example, “alkylene” is a divalent alkyl group connecting the groups to which it is attached.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

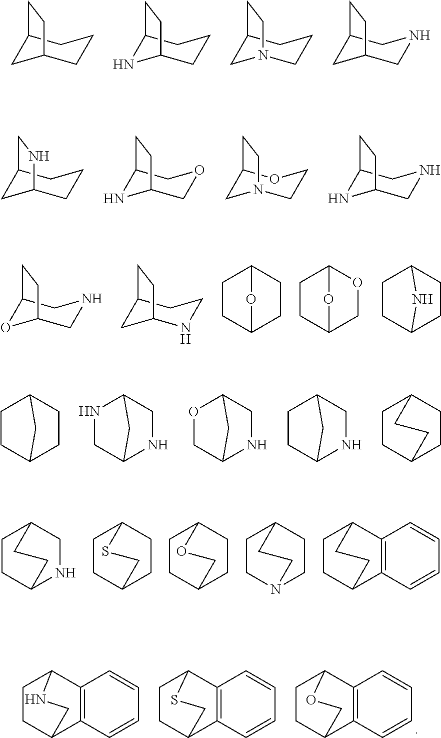

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o};
CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)(OR^{o})R^{o}; —P(O)R^{o}₂; —OP(O)R^{o}₂; —OP(O)(OR^{o})₂; —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “isomer” as used herein refers to a compound having the identical chemical formula but different structural or optical configurations. The term “stereoisomer” as used herein refers to and includes isomeric molecules that have the same molecular formula but differ in positioning of atoms and/or functional groups in the space. All stereoisomers of the present compounds (e.g., those which may exist due to asymmetric carbons on various substituents), including enantiomeric forms and diastereomeric forms, are contemplated within the scope of this disclosure. Therefore, unless otherwise stated, single stereochemical isomers as well as mixtures of enantiomeric, diastereomeric, and geometric (or conformational) isomers of the present compounds are within the scope of the disclosure.

The term “tautomer” as used herein refers to one of two or more structural isomers which exist in equilibrium and which are readily converted from one isomeric form to another. It is understood that tautomers encompass valence tautomers and proton tautomers (also known as prototropic tautomers). Valence tautomers include interconversions by reorganization of some of the bonding electrons. Proton tautomers include interconversions via migration of a proton, such as keto-enol and imine-enamine isomerizations. Unless otherwise stated, all tautomers of the compounds of the disclosure are within the scope of the disclosure.

The term “isotopic substitution” as used herein refers to the substitution of an atom with its isotope. The term “isotope” as used herein refers to an atom having the same atomic number as that of atoms dominant in nature but having a mass number (neutron number) different from the mass number of the atoms dominant in nature. It is understood that a compound with an isotopic substitution refers to a compound in which at least one atom contained therein is substituted with its isotope. Atoms that can be substituted with its isotope include, but are not limited to, hydrogen, carbon, and oxygen. Examples of the isotope of a hydrogen atom include ²H (also represented as D) and ³H. Examples of the isotope of a carbon atom include ¹³C and ¹⁴C. Examples of the isotope of an oxygen atom include ¹⁸O. Unless otherwise stated, all isotopic substitution of the compounds of the disclosure are within the scope of the disclosure. Such compounds are useful, for example, as analytical tools, as probes in biological assays, or as therapeutic agents in accordance with the present disclosure.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Exemplary pharmaceutically acceptable salts are found, e.g., in Berge, et al. (J. Pharm. Sci. 1977, 66(1), 1; and Gould, P. L., Int. J. Pharmaceutics 1986, 33, 201-217; (each hereby incorporated by reference in its entirety).

Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount”, or “therapeutically effective amount” as used herein is an amount of a compound that is sufficient to effect beneficial or desired results, including clinical results. As such, the effective amount may be sufficient, e.g., to reduce or ameliorate the severity and/or duration of afflictions related to PI3Kα signaling, or one or more symptoms thereof, prevent the advancement of conditions or symptoms related to afflictions related to PI3Kα signaling, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease or affliction, a stabilized (i.e., not worsening) state of disease or affliction, preventing spread of disease or affliction, delay or slowing of disease or affliction progression, amelioration or palliation of the disease or affliction state and remission (whether partial or total), whether detectable or undetectable. “Treatment” can also mean prolonging survival as compared to expected survival if not receiving treatment. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “in need thereof” refers to the need for symptomatic or asymptomatic relief from conditions related to PI3Kα signaling activity or that may otherwise be relieved by the compounds and/or compositions of the disclosure.

### 3. Description of Exemplary Embodiments

As described above, in some embodiments, the present disclosure provides a compound of formula I:

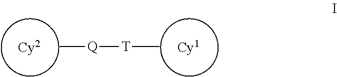

or a pharmaceutically acceptable salt thereof, wherein:
Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹;
Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R²;
Q is L^{Q};
T is a bivalent C₁₋₃ aliphatic chain substituted with q instances of R^{T};
each R¹ is independently -L¹-R^{1A};
each R² is independently -L²-R^{2A};
each R^{T} is independently -L^{T}-R^{TA}; or
two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC};
two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C};
two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C};
one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; or
one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC};
each of L¹, L², L^{Q}, and L^{T} is independently a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —N(R)C(NR)—, —N(R)C(NOR)—, —N(R)C(NCN)—, —C(O)N(R)—, —N(R)S(O)₂—, —S(O)₂N(R)—, —O—, —C(O)—, —OC(O)—, —C(O)O—, —S—, —S(O)—, or —S(O)₂—;
each R^{1A} is independently R^{A} or R^{B} substituted by r¹ instances of R^{1C};
each R^{2A} is independently R^{A} or R^{B} substituted by r² instances of R^{2C};
each RTA is independently R^{A} or R^{B} substituted by r³ instances of R^{TC};
each R^{L} is independently R^{A} or R^{B} substituted by r⁴ instances of R^{LC};
each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂;
each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur;
each instance of R^{1C}, R^{2C}, R^{TC}, R^{TTC}, R^{11C}, R^{22C}, R^{T1C}, R^{TLC}, and R^{LC} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur;
each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
each of n, m, q, p¹, p², p³, p⁴, p⁵, r¹, r², r³, and r⁴ is independently 0, 1, 2, 3, 4, or 5.

As defined generally above, Cy¹ is phenyl; naphthyl; cubanyl; adamantyl; a 3-7 membered saturated or partially unsaturated monocyclic carbocyclic ring; a 3-7 membered saturated or partially unsaturated monocyclic heterocyclic ring having 1-2 heteroatoms independently selected from nitrogen, oxygen, and sulfur; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; wherein Cy¹ is substituted with n instances of R¹.

In some embodiments, Cy¹ is phenyl, wherein Cy¹ is substituted with n instances of R¹. In some embodiments, Cy¹ is naphthyl, wherein Cy¹ is substituted with n instances of R¹. In some embodiments, Cy¹ is cubanyl, wherein Cy¹ is substituted with n instances of R¹. In some embodiments, Cy¹ is adamantyl, wherein Cy¹ is substituted with n instances of R¹. In some embodiments, Cy¹ is a 3-7 membered saturated or partially unsaturated monocyclic carbocyclic ring, wherein Cy¹ is substituted with n instances of R¹. In some embodiments, Cy¹ is a 3-7 membered saturated or partially unsaturated monocyclic heterocyclic ring having 1-2 heteroatoms independently selected from nitrogen, oxygen, and sulfur, wherein Cy¹ is substituted with n instances of R¹. In some embodiments, Cy¹ is a 5-6 membered monocyclic heteroaryl ring having 1-4 heteroatoms independently selected from nitrogen, oxygen, and sulfur, wherein Cy¹ is substituted with n instances of R¹. In some embodiments, Cy¹ is an 8-10 membered bicyclic heteroaryl ring having 1-4 heteroatoms independently selected from nitrogen, oxygen, and sulfur, wherein Cy¹ is substituted with n instances of R¹.

In some embodiments, Cy¹ is

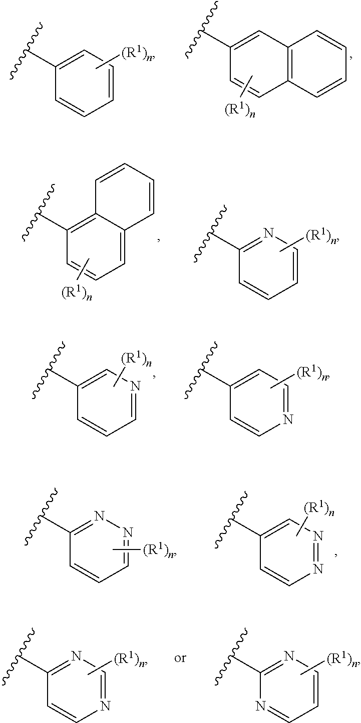

wherein R¹ and n are as defined in the embodiments and classes and subclasses herein. In some embodiments, Cy¹ is

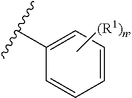

wherein R¹ and n are as defined in the embodiments and classes and subclasses herein. In some embodiments, Cy¹ is

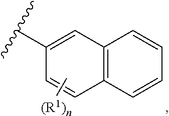

wherein R¹ and n are as defined in the embodiments and classes and subclasses herein. In some embodiments, Cy¹ is

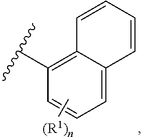

wherein R¹ and n are as defined in the embodiments and classes and subclasses herein. In some embodiments, Cy¹ is

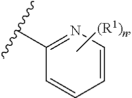

wherein R¹ and n are as defined in the embodiments and classes and subclasses herein. In some embodiments, Cy¹ is

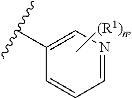

wherein R¹ and n are as defined in the embodiments and classes and subclasses herein. In some embodiments, Cy¹ is

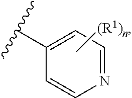

wherein R¹ and n are as defined in the embodiments and classes and subclasses herein. In some embodiments, Cy¹ is

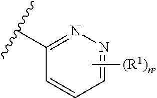

wherein R¹ and n are as defined in the embodiments and classes and subclasses herein. In some embodiments, Cy¹ is

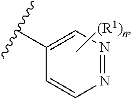

wherein R¹ and n are as defined in the embodiments and classes and subclasses herein. In some embodiments, Cy is

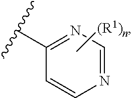

wherein R¹ and n are as defined in the embodiments and classes and subclasses herein. In some embodiments, Cy, is

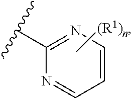

wherein R¹ and n are as defined in the embodiments and classes and subclasses herein.

In some embodiments, Cy¹ is

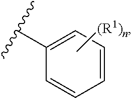

wherein R¹ is halogen and n is as defined in the embodiments and classes and subclasses herein. In some embodiments, Cy¹ is

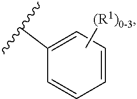

wherein R¹ is halogen. In some embodiments, Cy¹ is

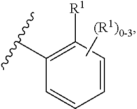

wherein R¹ is as defined in the embodiments and classes and subclasses herein. In some embodiments, Cy¹ is

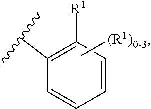

wherein R¹ is halogen. In some embodiments, Cy¹ is

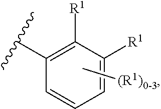

wherein R¹ is as defined in the embodiments and classes and subclasses herein. In some embodiments, Cy¹

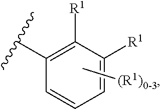

wherein R¹ is halogen. In some embodiments, Cy¹ is

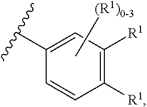

wherein R¹ is as defined in the embodiments and classes and subclasses herein. In some embodiments, Cy¹ is

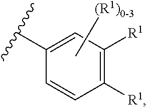

wherein R¹ is halogen. In some embodiments, Cy¹ is

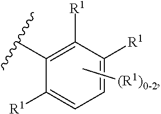

wherein R¹ is as defined in the embodiments and classes and subclasses herein. In some embodiments, Cy¹ is

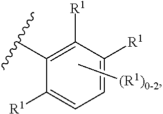

wherein R¹ is halogen.

In some embodiments, Cy¹ is

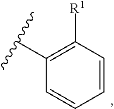

wherein R¹ is as defined in the embodiments and classes and subclasses herein. In some embodiments, Cy¹ is

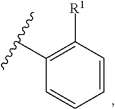

wherein R¹ is halogen. In some embodiments, Cy¹ is

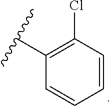 [SMILES: *c1ccccc1Cl]

In some embodiments, Cy¹ is

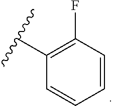 [SMILES: *c1ccccc1F]

In some embodiments, Cy¹ is

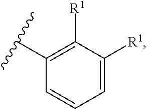

wherein R¹ is as defined in the embodiments and classes and subclasses herein. In some embodiments, Cy¹ is

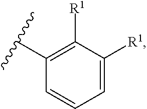

wherein R¹ is halogen. In some embodiments, Cy¹ is

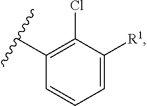

wherein R¹ is halogen. In some embodiments, Cy¹ is

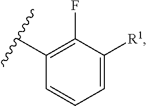

wherein R¹ is halogen. In some embodiments, Cy¹ is

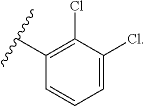 [SMILES: *c1cccc(Cl)c1Cl]

In some embodiments, Cy¹ is

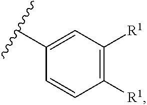

wherein R¹ is as defined in the embodiments and classes and subclasses herein. In some embodiments, Cy¹ is

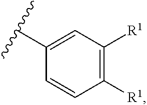

wherein R¹ is halogen. In some embodiments, Cy¹ is

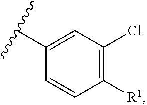

wherein R¹ is halogen. In some embodiments, Cy¹ is

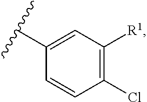

wherein R¹ is halogen. In some embodiments, Cy¹

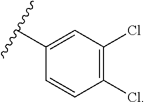 [SMILES: *c1ccc(Cl)c(Cl)c1]

In some embodiments, Cy¹ is

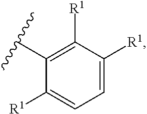

wherein R¹ is as defined in the embodiments and classes and subclasses herein. In some embodiments, Cy¹ is

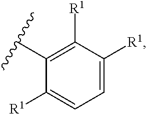

wherein R¹ is halogen. In some embodiments, Cy¹ is

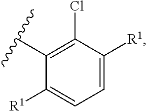

wherein R¹ is halogen. In some embodiments, Cy¹ is

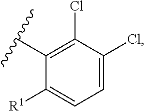

wherein R¹ is halogen. In some embodiments, Cy¹ is

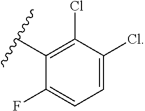 [SMILES: *c1c(F)ccc(Cl)c1Cl]

In some embodiments, Cy¹ is

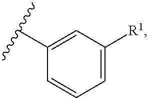

wherein R¹ is as defined in the embodiments and classes and subclasses herein. In some embodiments, Cy¹ is

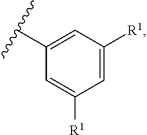

wherein R¹ is as defined in the embodiments and classes and subclasses herein. In some embodiments, Cy¹ is

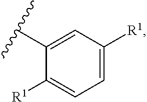

wherein R¹ is as defined in the embodiments and classes and subclasses herein. In some embodiments, Cy¹ is

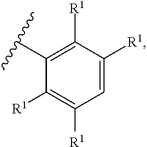

wherein R¹ is as defined in the embodiments and classes and subclasses herein. In some embodiments, Cy¹ is

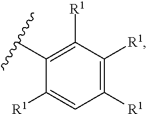

wherein R¹ is as defined in the embodiments and classes and subclasses herein. In some embodiments, Cy¹ is

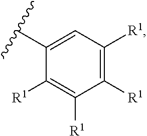

wherein R¹ is as defined in the embodiments and classes and subclasses herein. In some embodiments, Cy¹ is

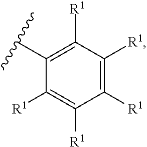

wherein R¹ is as defined in the embodiments and classes and subclasses herein.

In some embodiments, Cy¹ is an 8-10 membered bicyclic heteroaryl ring having 1-4 heteroatoms independently selected from nitrogen, oxygen, and sulfur; wherein Cy¹ is substituted with n instances of R¹ wherein R¹ is as defined in the embodiments and classes and subclasses herein. In some embodiments, Cy¹ is an 8-10 membered bicyclic heteroaryl ring having 1-2 heteroatoms independently selected from nitrogen, oxygen, and sulfur; wherein Cy¹ is substituted with n instances of R¹ wherein R¹ is as defined in the embodiments and classes and subclasses herein. In some embodiments, Cy¹ is

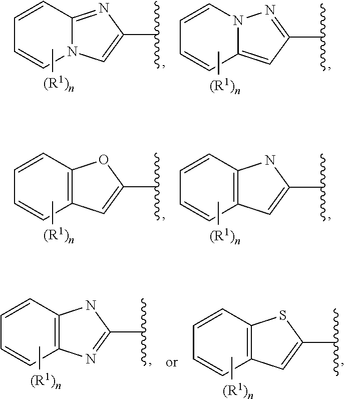

wherein n and R¹ are as defined in the embodiments and classes and subclasses herein. In some embodiments, Cy¹ is

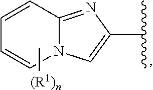

wherein n and R¹ are as defined in the embodiments and classes and subclasses herein. In some embodiments, Cy¹ is

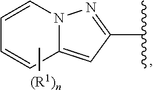

wherein n and R¹ are as defined in the embodiments and classes and subclasses herein. In some embodiments, Cy¹ is

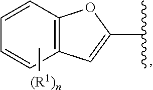

wherein n and R¹ are as defined in the embodiments and classes and subclasses herein. In some embodiments, Cy¹ is

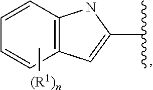

wherein n and R¹ are as defined in the embodiments and classes and subclasses herein. In some embodiments, Cy¹ is

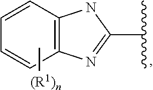

wherein n and R¹ are as defined in the embodiments and classes and subclasses herein. In some embodiments, Cy¹ is

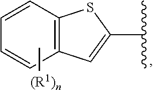

wherein n and R¹ are as defined in the embodiments and classes and subclasses herein. In some embodiments, Cy¹ is

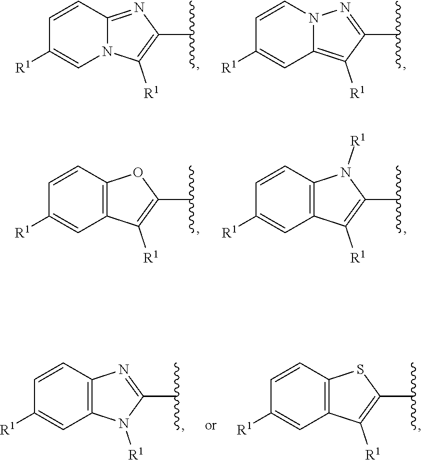

wherein n and R¹ are as defined in the embodiments and classes and subclasses herein. In some embodiments, Cy¹ is

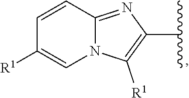

wherein n and R¹ are as defined in the embodiments and classes and subclasses herein. In some embodiments, Cy¹ is,

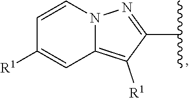

wherein n and R¹ are as defined in the embodiments and classes and subclasses herein. In some embodiments, Cy¹ is

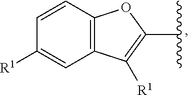

wherein n and R¹ are as defined in the embodiments and classes and subclasses herein. In some embodiments, Cy¹ is

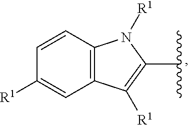

wherein n and R¹ are as defined in the embodiments and classes and subclasses herein. In some embodiments, Cy¹ is

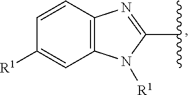

wherein n and R¹ are as defined in the embodiments and classes and subclasses herein. In some embodiments, Cy¹ is,

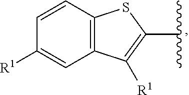

wherein n and R¹ are as defined in the embodiments and classes and subclasses herein. In some embodiments, Cy¹ is

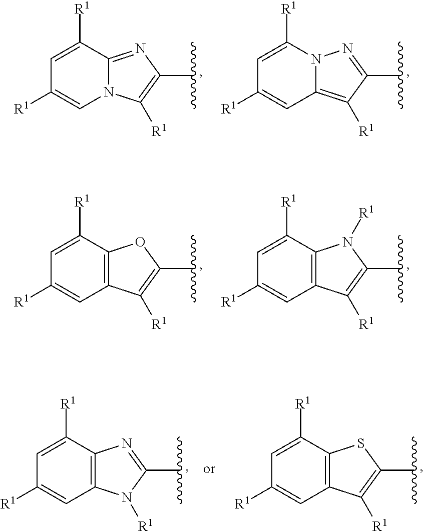

wherein n and R¹ are as defined in the embodiments and classes and subclasses herein. In some embodiments, Cy¹ is

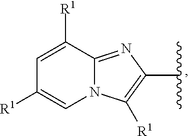

wherein n and R¹ are as defined in the embodiments and classes and subclasses herein. In some embodiments, Cy¹ is

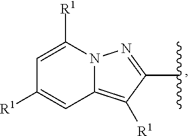

wherein n and R¹ are as defined in the embodiments and classes and subclasses herein. In some embodiments, Cy¹ is

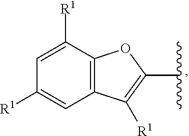

wherein n and R¹ are as defined in the embodiments and classes and subclasses herein. In some embodiments, Cy¹ is

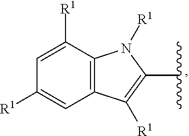

wherein n and R¹ are as defined in the embodiments and classes and subclasses herein. In some embodiments, Cy¹ is

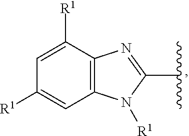

wherein n and R¹ are as defined in the embodiments and classes and subclasses herein. In some embodiments, Cy¹ is

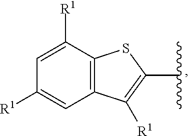

wherein n and R¹ are as defined in the embodiments and classes and subclasses herein.

In some embodiments, Cy¹ is selected from the groups depicted in the compounds in Table 1. In some embodiments, Cy¹ is not

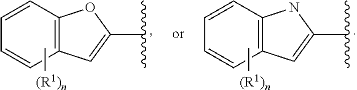

As defined generally above, Cy² is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein Cy² is substituted with m instances of R².

In some embodiments, Cy² is phenyl, wherein Cy² is substituted with m instances of R². In some embodiments, Cy² is naphthyl, wherein Cy² is substituted with m instances of R². In some embodiments, Cy² is cubanyl, wherein Cy² is substituted with m instances of R². In some embodiments, Cy² is adamantyl, wherein Cy² is substituted with m instances of R². In some embodiments, Cy² is a 5-6 membered monocyclic heteroaryl ring having 1-4 heteroatoms independently selected from nitrogen, oxygen, and sulfur, wherein Cy² is substituted with m instances of R². In some embodiments, Cy² is an 8-10 membered bicyclic heteroaryl ring having 1-4 heteroatoms independently selected from nitrogen, oxygen, and sulfur, wherein Cy² is substituted with m instances of R². In some embodiments, Cy² is a 3-7 membered saturated or partially unsaturated monocyclic carbocyclic ring, wherein Cy² is substituted with m instances of R². In some embodiments, Cy² is a 5-12 membered saturated or partially unsaturated bicyclic carbocyclic ring, wherein Cy² is substituted with m instances of R². In some embodiments, Cy² is a 3-7 membered saturated or partially unsaturated monocyclic heterocyclic ring having 1-2 heteroatoms independently selected from nitrogen, oxygen, and sulfur, wherein Cy² is substituted with m instances of R². In some embodiments, Cy² is a 7-12 membered saturated or partially unsaturated bicyclic heterocyclic ring having 1-4 heteroatoms independently selected from nitrogen, oxygen, and sulfur, wherein Cy² is substituted with m instances of R².

In some embodiments, Cy² is an 8-10 membered bicyclic heteroaryl ring having 1-4 heteroatoms independently selected from nitrogen, oxygen, and sulfur, wherein Cy² is substituted with m instances of R². In some embodiments, Cy² is a 9-10 membered bicyclic heteroaryl ring having 1-4 heteroatoms independently selected from nitrogen, oxygen, and sulfur, wherein Cy² is substituted with m instances of R². In some embodiments, Cy² is an 8-9 membered bicyclic heteroaryl ring having 1-4 heteroatoms independently selected from nitrogen, oxygen, and sulfur, wherein Cy² is substituted with m instances of R². In some embodiments, Cy² is an 8-membered bicyclic heteroaryl ring having 1-4 heteroatoms independently selected from nitrogen, oxygen, and sulfur, wherein Cy¹ is substituted with m instances of R². In some embodiments, Cy² is a 9-membered bicyclic heteroaryl ring having 1-4 heteroatoms independently selected from nitrogen, oxygen, and sulfur, wherein Cy² is substituted with m instances of R². In some embodiments, Cy² is a 10-membered bicyclic heteroaryl ring having 1-4 heteroatoms independently selected from nitrogen, oxygen, and sulfur, wherein Cy² is substituted with m instances of R².

In some embodiments, Cy² is a 3-7 membered saturated or partially unsaturated monocyclic carbocyclic ring, wherein Cy² is substituted with m instances of R². In some embodiments, Cy² is a 4-6 membered saturated or partially unsaturated monocyclic carbocyclic ring, wherein Cy² is substituted with m instances of R². In some embodiments, Cy² is a 4-5 membered saturated or partially unsaturated monocyclic carbocyclic ring, wherein Cy² is substituted with m instances of R². In some embodiments, Cy² is a 5-6 membered saturated or partially unsaturated monocyclic carbocyclic ring, wherein Cy² is substituted with m instances of R². In some embodiments, Cy² is a 4-membered saturated or partially unsaturated monocyclic carbocyclic ring, wherein Cy² is substituted with m instances of R². In some embodiments, Cy² is a 5-membered saturated or partially unsaturated monocyclic carbocyclic ring, wherein Cy² is substituted with m instances of R². In some embodiments, Cy² is a 6-membered saturated or partially unsaturated monocyclic carbocyclic ring, wherein Cy² is substituted with m instances of R².

In some embodiments, Cy² is

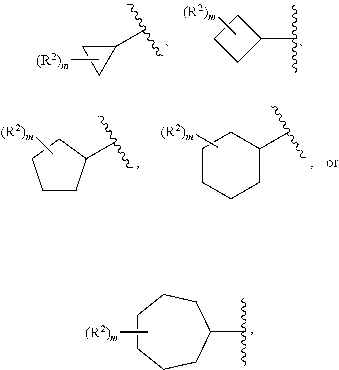

wherein R² and m are as defined in embodiments and classes and subclasses herein. In some embodiments, Cy² is

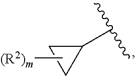

wherein R² and m are as defined in embodiments and classes and subclasses herein. In some embodiments, Cy² is

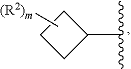

wherein R² and m are as defined in embodiments and classes and subclasses herein. In some embodiments, Cy² is

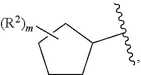

wherein R² and m are as defined in embodiments and classes and subclasses herein. In some embodiments, Cy² is

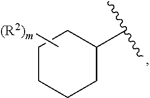

wherein R² and m are as defined in embodiments and classes and subclasses herein. In some embodiments, Cy² is

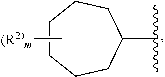

wherein R² and m are as defined in embodiments and classes and subclasses herein.

In some embodiments, Cy² is

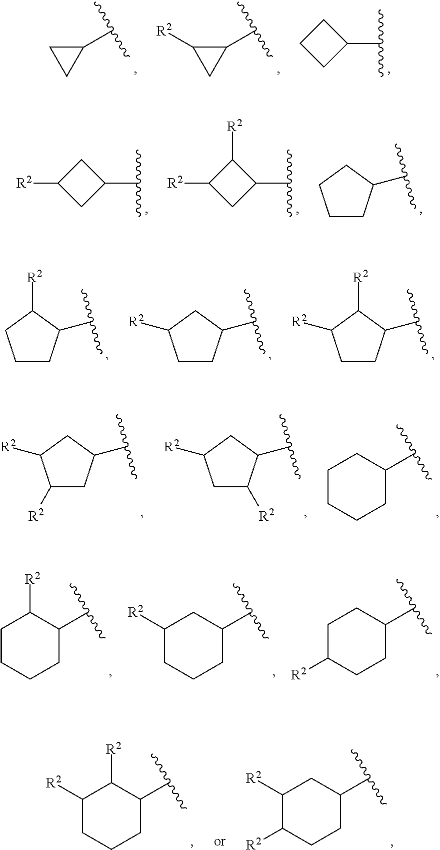

wherein R² is as defined in embodiments and classes and subclasses herein. In some embodiments, Cy² is

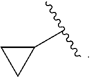 [SMILES: *C1CC1]

In some embodiments, Cy² is

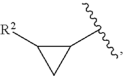

wherein R² is as defined in embodiments and classes and subclasses herein. In some embodiments, Cy² is

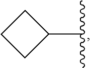

wherein R² is as defined in embodiments and classes and subclasses herein. In some embodiments, Cy² is

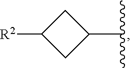

wherein R² is as defined in embodiments and classes and subclasses herein. In some embodiments, Cy² is

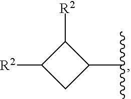

wherein R² is as defined in embodiments and classes and subclasses herein. In some embodiments, Cy² is

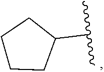

wherein R² is as defined in embodiments and classes and subclasses herein. In some embodiments, Cy² is

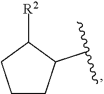

wherein R² is as defined in embodiments and classes and subclasses herein. In some embodiments, Cy² is

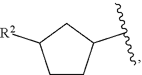

wherein R² is as defined in embodiments and classes and subclasses herein. In some embodiments, Cy² is

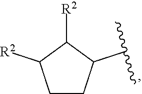

wherein R² is as defined in embodiments and classes and subclasses herein. In some embodiments, Cy² is

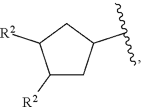

wherein R² is as defined in embodiments and classes and subclasses herein. In some embodiments, Cy² is

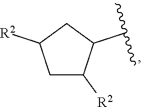

wherein R² is as defined in embodiments and classes and subclasses herein. In some embodiments, Cy² is

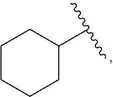

wherein R² is as defined in embodiments and classes and subclasses herein. In some embodiments, Cy² is

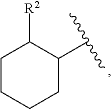

wherein R² is as defined in embodiments and classes and subclasses herein. In some embodiments, Cy² is

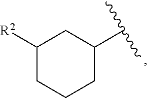

wherein R² is as defined in embodiments and classes and subclasses herein. In some embodiments, Cy² is

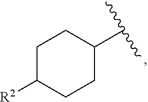

wherein R² is as defined in embodiments and classes and subclasses herein. In some embodiments, Cy² is

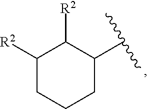

wherein R² is as defined in embodiments and classes and subclasses herein. In some embodiments, Cy² is

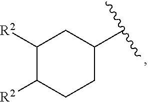

wherein R² is as defined in embodiments and classes and subclasses herein.

In some embodiments, Cy² is a 7-12 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-10 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-9 membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is an 8-membered saturated or partially unsaturated bicyclic heterocyclic ring having 1-4 heteroatoms independently selected from nitrogen, oxygen, and sulfur, wherein Cy² is substituted with m instances of R². In some embodiments, Cy² is a 9-membered saturated or partially unsaturated bicyclic heterocyclic ring having 1-4 heteroatoms independently selected from nitrogen, oxygen, and sulfur, wherein Cy² is substituted with m instances of R².

In some embodiments, Cy² is

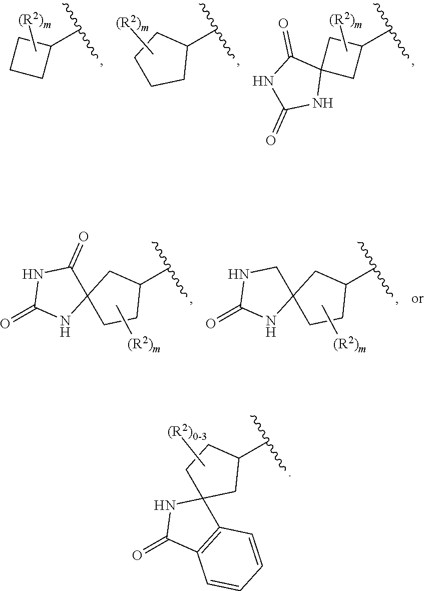

In some embodiments, Cy² is

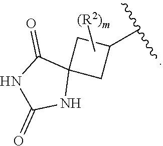

In some embodiments, Cy² is

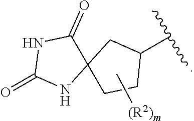

In some embodiments, Cy² is

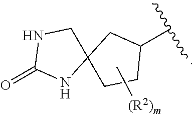

In some embodiments, Cy² is

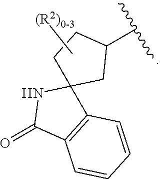

In some embodiments, Cy² is

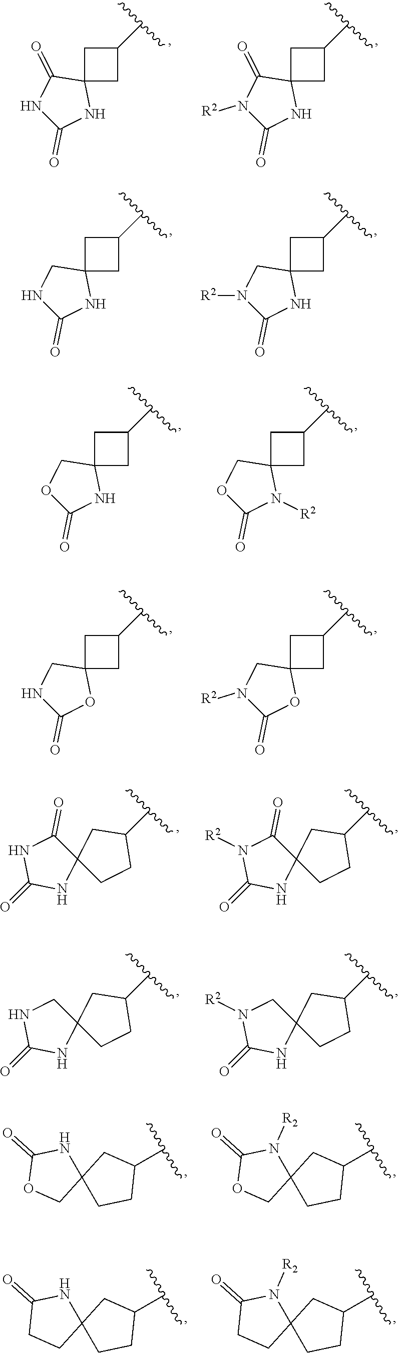

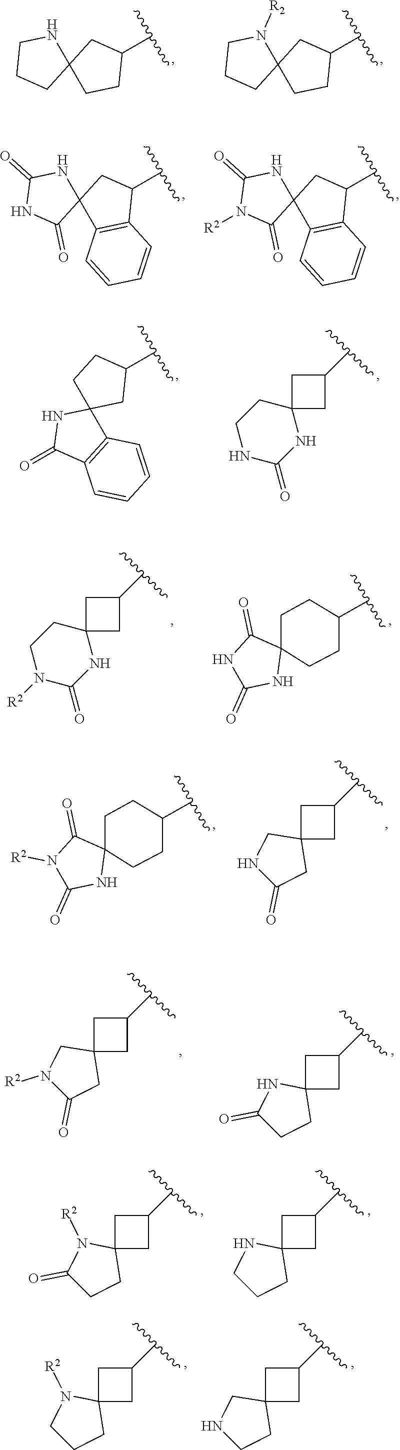

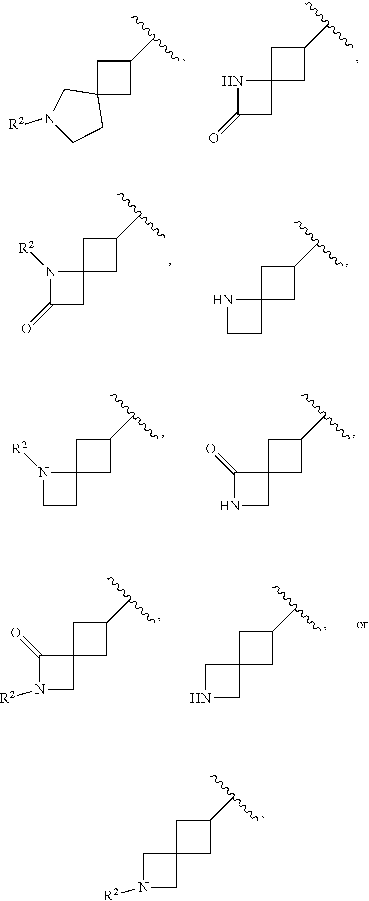

wherein R² is as defined in embodiments and classes and subclasses herein.

In some embodiments, Cy² is

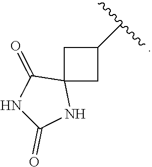 [SMILES: *C1CC2(C1)NC(=O)NC2=O]

In some embodiments, Cy² is

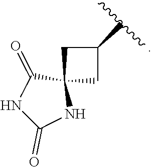 [SMILES: *[C@H]1C[C@]2(C1)NC(=O)NC2=O]

In some embodiments, Cy² is

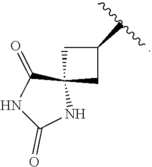 [SMILES: *[C@H]1C[C@@]2(C1)NC(=O)NC2=O]

In some embodiments, Cy² is

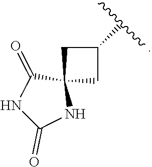 [SMILES: *[C@H]1C[C@@]2(C1)NC(=O)NC2=O]

In some embodiments, Cy² is

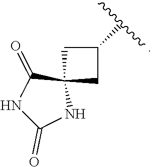 [SMILES: *[C@H]1C[C@]2(C1)NC(=O)NC2=O]

In some embodiments, Cy² is

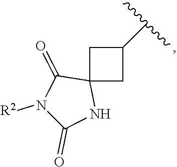

wherein R² is as defined in embodiments and classes and subclasses herein. In some embodiments, Cy² is

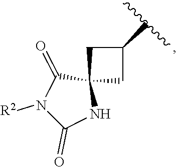

wherein R² is as defined in embodiments and classes and subclasses herein. In some embodiments, Cy² is

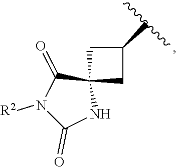

wherein R² is as defined in embodiments and classes and subclasses herein. In some embodiments, Cy² is

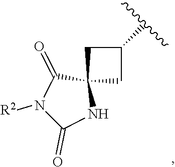

wherein R² is as defined in embodiments and classes and subclasses herein. In some embodiments, Cy² is

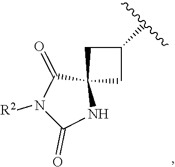

wherein R² is as defined in embodiments and classes and subclasses herein.

In some embodiments, Cy² is

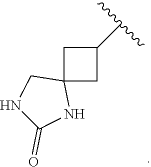 [SMILES: *C1CC2(CNC(=O)N2)C1]

In some embodiments, Cy² is

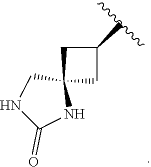 [SMILES: *[C@H]1C[C@]2(CNC(=O)N2)C1]

In some embodiments, Cy² is

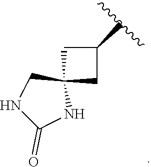 [SMILES: *[C@H]1C[C@@]2(CNC(=O)N2)C1]

In some embodiments, Cy² is

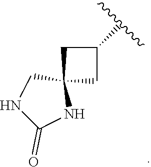 [SMILES: *[C@H]1C[C@@]2(CNC(=O)N2)C1]

In some embodiments, Cy² is

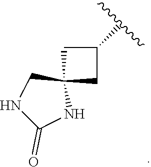 [SMILES: *[C@H]1C[C@]2(CNC(=O)N2)C1]

In some embodiments, Cy² is

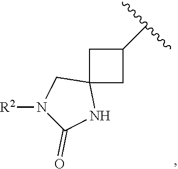

wherein R² is as defined in embodiments and classes and subclasses herein. In some embodiments, Cy² is

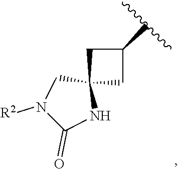

wherein R² is as defined in embodiments and classes and subclasses herein. In some embodiments, Cy² is

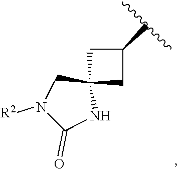

wherein R² is as defined in embodiments and classes and subclasses herein. In some embodiments, Cy² is

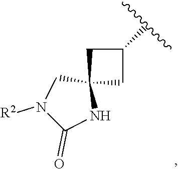

wherein R² is as defined in embodiments and classes and subclasses herein. In some embodiments, Cy² is

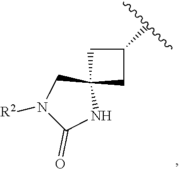

wherein R² is as defined in embodiments and classes and subclasses herein.

In some embodiments, Cy² is

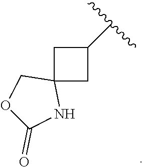 [SMILES: *C1CC2(COC(=O)N2)C1]

In some embodiments, Cy² is

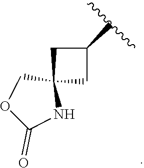 [SMILES: *[C@H]1C[C@]2(COC(=O)N2)C1]

In some embodiments, Cy² is

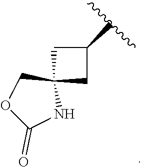 [SMILES: *[C@H]1C[C@@]2(COC(=O)N2)C1]

In some embodiments, Cy² is

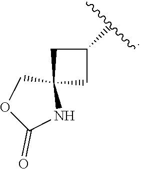 [SMILES: *[C@H]1C[C@@]2(COC(=O)N2)C1]

In some embodiments, Cy² is

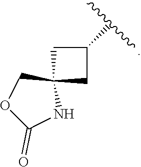 [SMILES: *[C@H]1C[C@]2(COC(=O)N2)C1]

In some embodiments, Cy² is

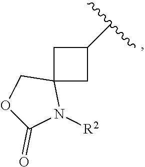

wherein R² is as defined in embodiments and classes and subclasses herein. In some embodiments, Cy² is

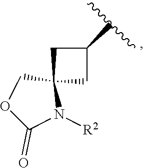

wherein R² is as defined in embodiments and classes and subclasses herein. In some embodiments, Cy² is

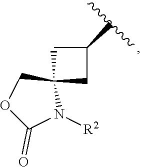

wherein R² is as defined in embodiments and classes and subclasses herein. In some embodiments, Cy² is

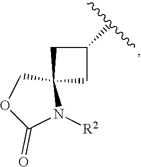

wherein R² is as defined in embodiments and classes and subclasses herein. In some embodiments, Cy² is

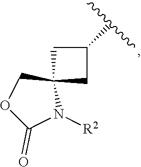

wherein R² is as defined in embodiments and classes and subclasses herein.

In some embodiments, Cy² is

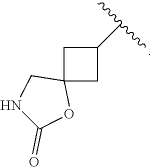 [SMILES: *C1CC2(CNC(=O)O2)C1]

In some embodiments, Cy² is

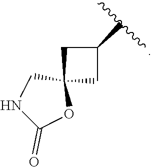 [SMILES: *[C@H]1C[C@]2(CNC(=O)O2)C1]

In some embodiments, Cy² is

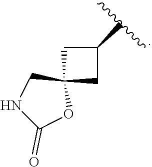 [SMILES: *[C@H]1C[C@@]2(CNC(=O)O2)C1]

In some embodiments, Cy² is

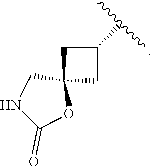 [SMILES: *[C@H]1C[C@@]2(CNC(=O)O2)C1]

In some embodiments, Cy² is

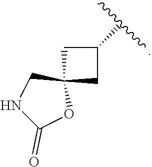 [SMILES: *[C@H]1C[C@]2(CNC(=O)O2)C1]

In some embodiments, Cy² is

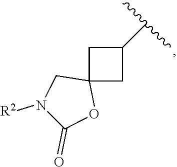

wherein R² is as defined in embodiments and classes and subclasses herein. In some embodiments, Cy² is

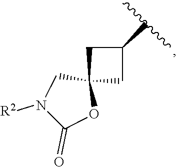

wherein R² is as defined in embodiments and classes and subclasses herein. In some embodiments, Cy² is

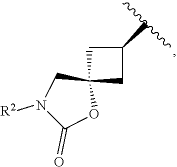

wherein R² is as defined in embodiments and classes and subclasses herein. In some embodiments, Cy² is

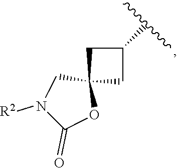

wherein R² is as defined in embodiments and classes and subclasses herein. In some embodiments, Cy² is

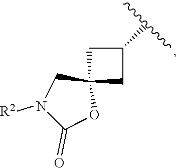

wherein R² is as defined in embodiments and classes and subclasses herein.

In some embodiments, Cy² is

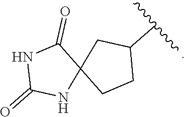 [SMILES: *C1CCC2(C1)NC(=O)NC2=O]

In some embodiments, Cy² is

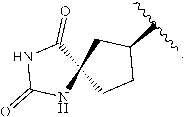 [SMILES: *[C@H]1CC[C@]2(C1)NC(=O)NC2=O]

In some embodiments, Cy² is

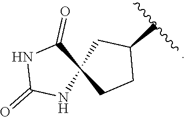 [SMILES: *[C@H]1CC[C@@]2(C1)NC(=O)NC2=O]

In some embodiments, Cy²

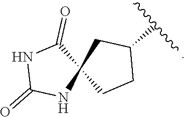 [SMILES: *[C@@H]1CC[C@]2(C1)NC(=O)NC2=O]

In some embodiments, Cy² is

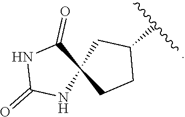 [SMILES: *[C@@H]1CC[C@@]2(C1)NC(=O)NC2=O]

In some embodiments, Cy² is

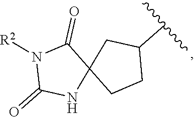

wherein R² is as defined in embodiments and classes and subclasses herein. In some embodiments, Cy² is

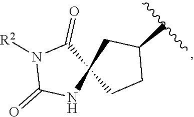

wherein R² is as defined in embodiments and classes and subclasses herein. In some embodiments, Cy² is

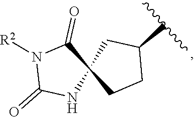

wherein R² is as defined in embodiments and classes and subclasses herein. In some embodiments, Cy²

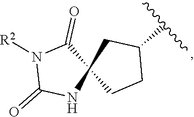

wherein R² is as defined in embodiments and classes and subclasses herein. In some embodiments, Cy¹ is

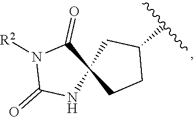

wherein R² is as defined in embodiments and classes and subclasses herein.

In some embodiments, Cy² is

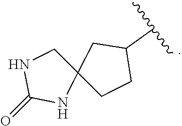 [SMILES: *C1CCC2(CNC(=O)N2)C1]

In some embodiments, Cy² is

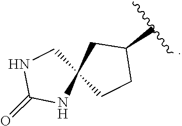 [SMILES: *[C@H]1CC[C@]2(CNC(=O)N2)C1]

In some embodiments, Cy² is

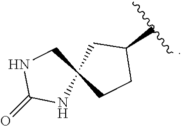 [SMILES: *[C@H]1CC[C@@]2(CNC(=O)N2)C1]

In some embodiments, Cy² is

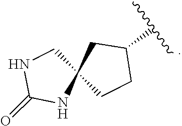 [SMILES: *[C@@H]1CC[C@]2(CNC(=O)N2)C1]

In some embodiments, Cy² is

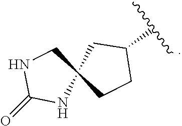 [SMILES: *[C@@H]1CC[C@@]2(CNC(=O)N2)C1]

In some embodiments, Cy² is

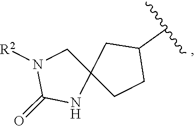

wherein R² is as defined in embodiments and classes and subclasses herein. In some embodiments, Cy² is

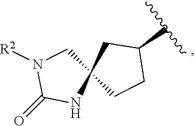

wherein R² is as defined in embodiments and classes and subclasses herein. In some embodiments, Cy² is

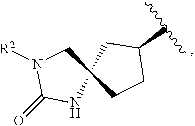

wherein R² is as defined in embodiments and classes and subclasses herein. In some embodiments, Cy² is

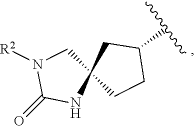

wherein R² is as defined in embodiments and classes and subclasses herein. In some embodiments, Cy² is

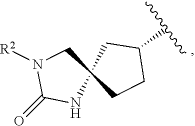

wherein R² is as defined in embodiments and classes and subclasses herein.

In some embodiments, Cy² is

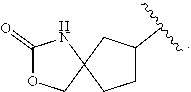 [SMILES: *C1CCC2(COC(=O)N2)C1]

In some embodiments, Cy² is

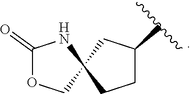 [SMILES: *[C@H]1CC[C@]2(COC(=O)N2)C1]

In some embodiments, Cy² is

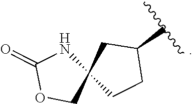 [SMILES: *[C@H]1CC[C@@]2(COC(=O)N2)C1]

In some embodiments, Cy² is

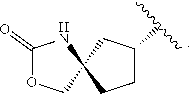 [SMILES: *[C@@H]1CC[C@]2(COC(=O)N2)C1]

In some embodiments, Cy² is

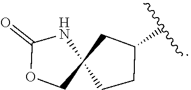 [SMILES: *[C@@H]1CC[C@@]2(COC(=O)N2)C1]

In some embodiments, Cy² is

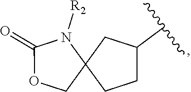

wherein R² is as defined in embodiments and classes and subclasses herein. In some embodiments, Cy² is

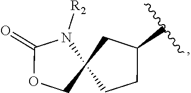

wherein R² is as defined in embodiments and classes and subclasses herein. In some embodiments, Cy² is

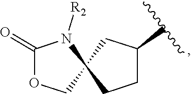

wherein R² is as defined in embodiments and classes and subclasses herein. In some embodiments, Cy² is

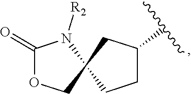

wherein R² is as defined in embodiments and classes and subclasses herein. In some embodiments, Cy² is

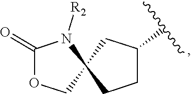

wherein R² is as defined in embodiments and classes and subclasses herein.

In some embodiments, Cy² is

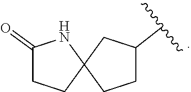 [SMILES: *C1CCC2(CCC(=O)N2)C1]

In some embodiments, Cy² is

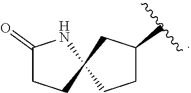 [SMILES: *[C@H]1CC[C@@]2(CCC(=O)N2)C1]

In some embodiments, Cy² is

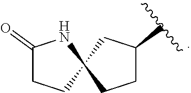 [SMILES: *[C@H]1CC[C@]2(CCC(=O)N2)C1]

In some embodiments, Cy² is

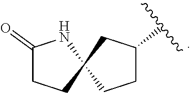 [SMILES: *[C@@H]1CC[C@@]2(CCC(=O)N2)C1]

In some embodiments, Cy² is

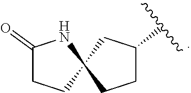 [SMILES: *[C@@H]1CC[C@]2(CCC(=O)N2)C1]

In some embodiments, Cy² is

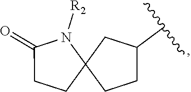

wherein R² is as defined in embodiments and classes and subclasses herein. In some embodiments, Cy² is

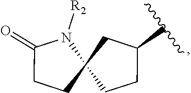

wherein R² is as defined in embodiments and classes and subclasses herein. In some embodiments, Cy² is

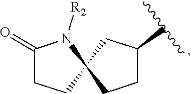

wherein R² is as defined in embodiments and classes and subclasses herein. In some embodiments, Cy² is

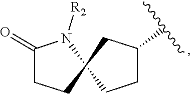

wherein R² is as defined in embodiments and classes and subclasses herein. In some embodiments, Cy² is

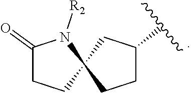

wherein R² is as defined in embodiments and classes and subclasses herein.

In some embodiments, Cy² is

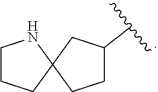 [SMILES: *C1CCC2(CCCN2)C1]

In some embodiments, Cy² is

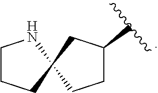 [SMILES: *[C@H]1CC[C@@]2(CCCN2)C1]

In some embodiments, Cy² is

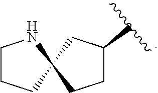 [SMILES: *[C@H]1CC[C@]2(CCCN2)C1]

In some embodiments, Cy² is

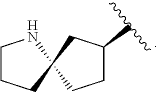 [SMILES: *[C@H]1CC[C@@]2(CCCN2)C1]

In some embodiments, Cy² is

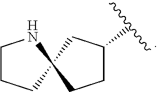 [SMILES: *[C@@H]1CC[C@]2(CCCN2)C1]

In some embodiments, Cy² is

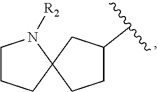

wherein R² is as defined in embodiments and classes and subclasses herein. In some embodiments, Cy² is

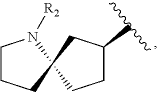

wherein R² is as defined in embodiments and classes and subclasses herein. In some embodiments, Cy² is

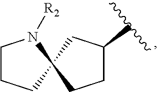

wherein R² is as defined in embodiments and classes and subclasses herein. In some embodiments, Cy² is

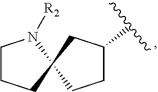

wherein R² is as defined in embodiments and classes and subclasses herein. In some embodiments, Cy² is

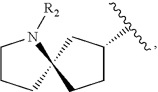

wherein R² is as defined in embodiments and classes and subclasses herein.

In some embodiments, Cy² is

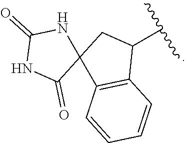 [SMILES: *C1CC2(NC(=O)NC2=O)c2ccccc21]

In some embodiments, Cy² is

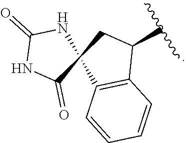 [SMILES: *[C@@H]1C[C@@]2(NC(=O)NC2=O)c2ccccc21]

In some embodiments, Cy² is

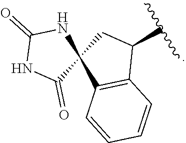 [SMILES: *[C@@H]1C[C@]2(NC(=O)NC2=O)c2ccccc21]

In some embodiments, Cy² is

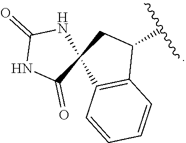 [SMILES: *[C@H]1C[C@@]2(NC(=O)NC2=O)c2ccccc21]

In some embodiments, Cy² is

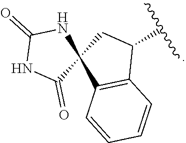 [SMILES: *[C@H]1C[C@]2(NC(=O)NC2=O)c2ccccc21]

In some embodiments, Cy² is

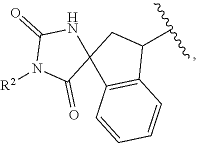

wherein R² is as defined in embodiments and classes and subclasses herein. In some embodiments, Cy² is

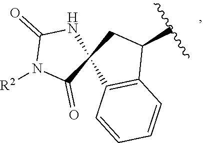

wherein R² is as defined in embodiments and classes and subclasses herein. In some embodiments, Cy² is

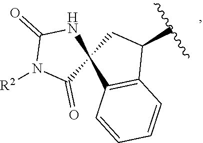

wherein R² is as defined in embodiments and classes and subclasses herein. In some embodiments, Cy² is

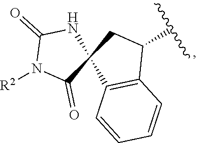

wherein R² is as defined in embodiments and classes and subclasses herein. In some embodiments, Cy² is

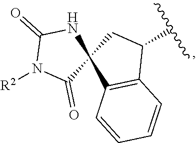

wherein R² is a defined in embodiments and classes and subclasses herein.

In some embodiments, Cy² is

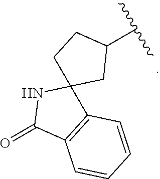 [SMILES: *C1CCC2(C1)NC(=O)c1ccccc12]

In some embodiments, Cy² is

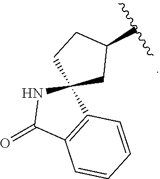 [SMILES: *[C@@H]1CC[C@@]2(C1)NC(=O)c1ccccc12]

In some embodiments, Cy² is

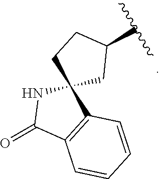 [SMILES: *[C@@H]1CC[C@]2(C1)NC(=O)c1ccccc12]

In some embodiments, Cy² is

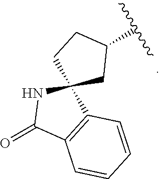 [SMILES: *[C@H]1CC[C@@]2(C1)NC(=O)c1ccccc12]

In some embodiments, Cy² is

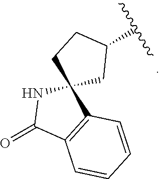 [SMILES: *[C@H]1CC[C@]2(C1)NC(=O)c1ccccc12]

In some embodiments, Cy² is

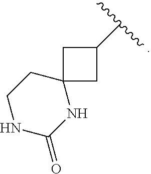 [SMILES: *C1CC2(CCNC(=O)N2)C1]

In some embodiments, Cy² is

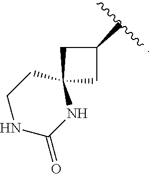 [SMILES: *[C@H]1C[C@]2(CCNC(=O)N2)C1]

In some embodiments, Cy² is

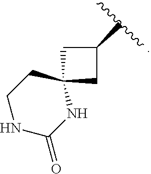 [SMILES: *[C@H]1C[C@@]2(CCNC(=O)N2)C1]

In some embodiments Cy² is

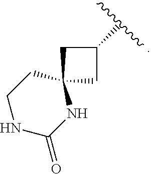 [SMILES: *[C@H]1C[C@@]2(CCNC(=O)N2)C1]

In some embodiments, Cy² is

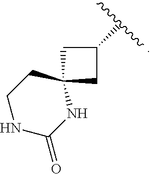 [SMILES: *[C@H]1C[C@]2(CCNC(=O)N2)C1]

In some embodiments, Cy² is

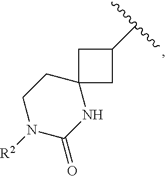

wherein R² is as defined in embodiments and classes and subclasses herein. In some embodiments, Cy² is

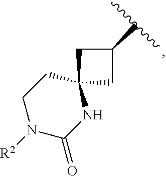

wherein R² is as defined in embodiments and classes and subclasses herein. In some embodiments, Cy² is

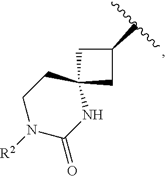

wherein R² is as defined in embodiments and classes and subclasses herein. In some embodiments, Cy² is

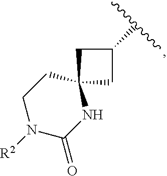

wherein R² is as defined in embodiments and classes and subclasses herein. In some embodiments, Cy² is

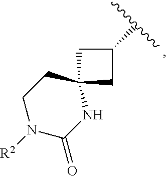

wherein R² is as defined in embodiments and classes and subclasses herein.

In some embodiments, Cy² is

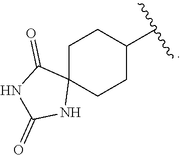 [SMILES: *C1CCC2(CC1)NC(=O)NC2=O]

In some embodiments, Cy² is

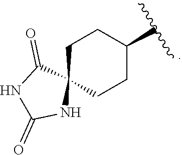 [SMILES: *[C@H]1CC[C@]2(CC1)NC(=O)NC2=O]

In some embodiments, Cy² is

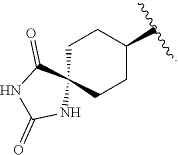 [SMILES: *[C@H]1CC[C@@]2(CC1)NC(=O)NC2=O]

In some embodiments, Cy² is

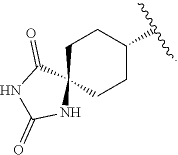 [SMILES: *[C@H]1CC[C@@]2(CC1)NC(=O)NC2=O]

In some embodiments, Cy² is

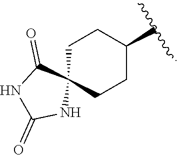 [SMILES: *[C@H]1CC[C@@]2(CC1)NC(=O)NC2=O]

In some embodiments, Cy² is

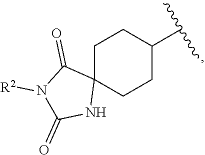

wherein R² is as defined in embodiments and classes and subclasses herein. In some embodiments, Cy² is

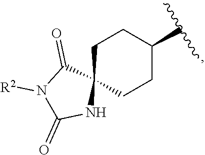

wherein R² is as defined in embodiments and classes and subclasses herein. In some embodiments, Cy² is

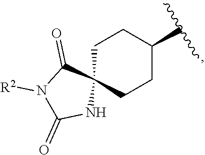

wherein R² is as defined in embodiments and classes and subclasses herein. In some embodiments, Cy² is

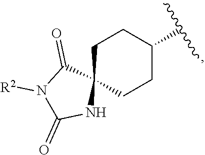

wherein R² is as defined in embodiments and classes and subclasses herein. In some embodiments, Cy² is

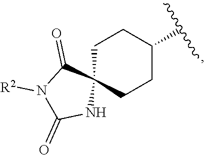

wherein R² is as defined in embodiments and classes and subclasses herein.

In some embodiments, Cy² is

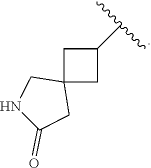 [SMILES: *C1CC2(CNC(=O)C2)C1]

In some embodiments, Cy² is

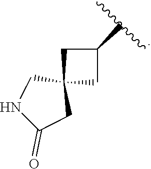 [SMILES: *[C@H]1C[C@]2(CNC(=O)C2)C1]

In some embodiments, Cy² is

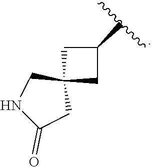 [SMILES: *[C@H]1C[C@@]2(CNC(=O)C2)C1]

In some embodiments, Cy² is

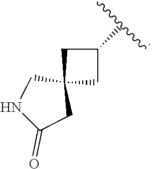 [SMILES: *[C@H]1C[C@@]2(CNC(=O)C2)C1]

In some embodiments, Cy² is

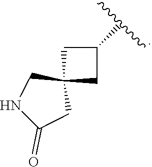 [SMILES: *[C@H]1C[C@]2(CNC(=O)C2)C1]

In some embodiments, Cy² is

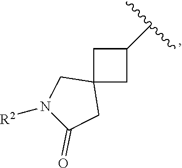

wherein R² is as defined in embodiments and classes and subclasses herein. In some embodiments, Cy² is

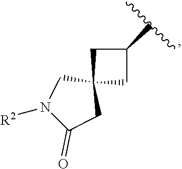

wherein R² is as defined in embodiments and classes and subclasses herein. In some embodiments, Cy² is

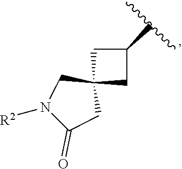

wherein R² is as defined in embodiments and classes and subclasses herein. In some embodiments, Cy² is

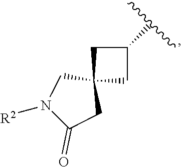

wherein R² is as defined in embodiments and classes and subclasses herein. In some embodiments, Cy² is

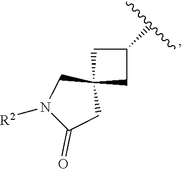

wherein R² is as defined in classes and subclasses herein.

In some embodiments, Cy² is

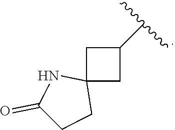 [SMILES: *C1CC2(CCC(=O)N2)C1]

In some embodiments, Cy² is

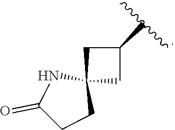 [SMILES: *[C@H]1C[C@@]2(CCC(=O)N2)C1]

In some embodiments, Cy² is

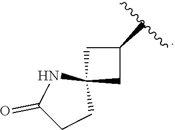 [SMILES: *[C@H]1C[C@]2(CCC(=O)N2)C1]

In some embodiments, Cy² is

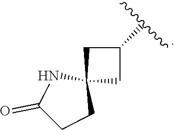 [SMILES: *[C@H]1C[C@]2(CCC(=O)N2)C1]

In some embodiments, Cy² is

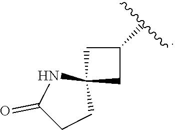 [SMILES: *[C@H]1C[C@@]2(CCC(=O)N2)C1]

In some embodiments, Cy² is

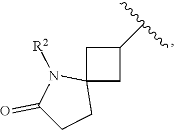

wherein R² is as defined in embodiments and classes and subclasses herein. In some embodiments, Cy² is

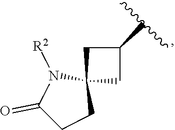

wherein R² is as defined in embodiments and classes and subclasses herein. In some embodiments, Cy² is

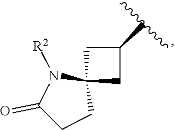

wherein R² is as defined in embodiments and classes and subclasses herein. In some embodiments, Cy² is

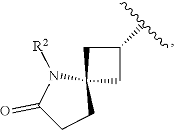

wherein R² is as defined in embodiments and classes and subclasses herein. In some embodiments, Cy² is

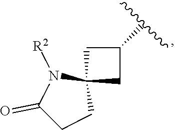

wherein R² is as defined in classes and subclasses herein.

In some embodiments, Cy² is

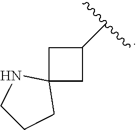 [SMILES: *C1CC2(CCCN2)C1]

In some embodiments, Cy² is

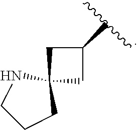 [SMILES: *[C@H]1C[C@@]2(CCCN2)C1]

In some embodiments, Cy² is

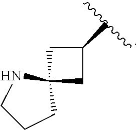 [SMILES: *[C@H]1C[C@]2(CCCN2)C1]

In some embodiments, Cy² is

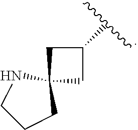 [SMILES: *[C@H]1C[C@]2(CCCN2)C1]

In some embodiments, Cy² is

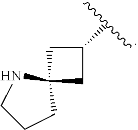 [SMILES: *[C@H]1C[C@@]2(CCCN2)C1]

In some embodiments, Cy² is

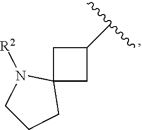

wherein R² is as defined in embodiments and classes and subclasses herein. In some embodiments, Cy² is

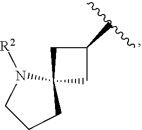

wherein R² is as defined in embodiments and classes and subclasses herein. In some embodiments, Cy² is

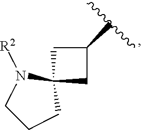

wherein R² is as defined in embodiments and classes and subclasses herein. In some embodiments, Cy² is

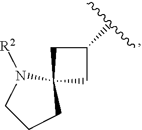

wherein R² is as defined in embodiments and classes and subclasses herein. In some embodiments, Cy² is

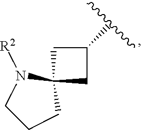

wherein R² is as defined in embodiments and classes and subclasses herein.

In some embodiments, Cy² is

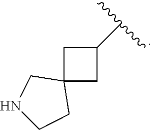 [SMILES: *C1CC2(CCNC2)C1]

In some embodiments, Cy² is

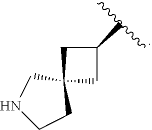 [SMILES: *[C@H]1C[C@@]2(CCNC2)C1]

In some embodiments, Cy² is

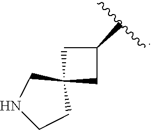 [SMILES: *[C@H]1C[C@]2(CCNC2)C1]

In some embodiments, Cy² is

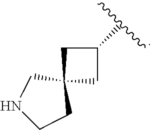 [SMILES: *[C@H]1C[C@]2(CCNC2)C1]

In some embodiments, Cy² is

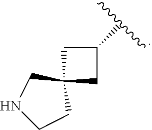 [SMILES: *[C@H]1C[C@@]2(CCNC2)C1]

In some embodiments, Cy² is

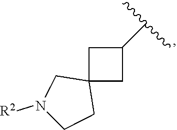

wherein R² is as defined in embodiments and classes and subclasses herein. In some embodiments, Cy² is

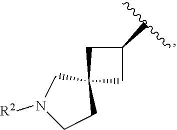

wherein R² is as defined in embodiments and classes and subclasses herein. In some embodiments, Cy² is

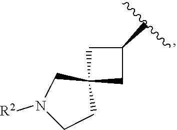

wherein R² is as defined in embodiments and classes and subclasses herein. In some embodiments, Cy² is

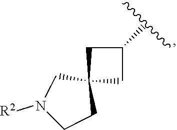

wherein R² is as defined in embodiments and classes and subclasses herein. In some embodiments, Cy² is

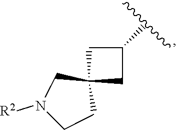

wherein R² is as defined in embodiments and classes and subclasses herein.

In some embodiments, Cy² is

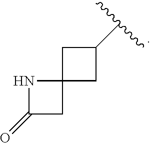 [SMILES: *C1CC2(CC(=O)N2)C1]

In some embodiments, Cy² is

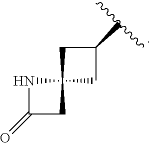 [SMILES: *[C@H]1C[C@@]2(CC(=O)N2)C1]

In some embodiments, Cy² is

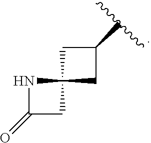 [SMILES: *[C@H]1C[C@]2(CC(=O)N2)C1]

In some embodiments, Cy² is

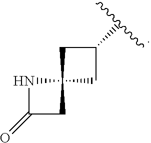 [SMILES: *[C@H]1C[C@]2(CC(=O)N2)C1]

In some embodiments, Cy² is

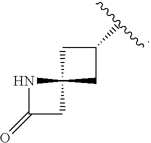 [SMILES: *[C@H]1C[C@@]2(CC(=O)N2)C1]

In some embodiments, Cy² is

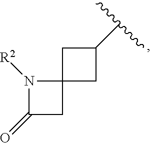

wherein R² is as defined in embodiments and classes and subclasses herein. In some embodiments, Cy² is

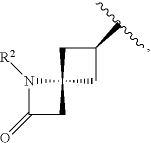

wherein R² is as defined in embodiments and classes and subclasses herein. In some embodiments, Cy² is

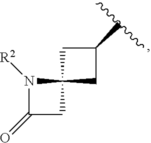

wherein R² is as defined in embodiments and classes and subclasses herein. In some embodiments, Cy² is

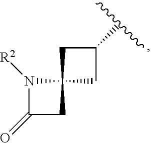

wherein R² is as defined in embodiments and classes and subclasses herein. In some embodiments, Cy² is

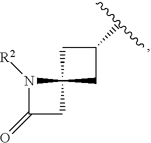

wherein R is as defined in embodiments and classes and subclasses herein.

In some embodiments, Cy² is

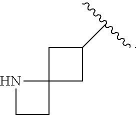 [SMILES: *C1CC2(CCN2)C1]

In some embodiments, Cy² is

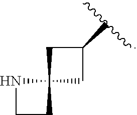 [SMILES: *[C@H]1C[C@@]2(CCN2)C1]

In some embodiments, Cy² is

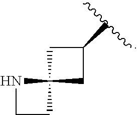 [SMILES: *[C@H]1C[C@]2(CCN2)C1]

In some embodiments, Cy² is

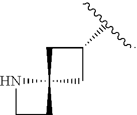 [SMILES: *[C@H]1C[C@]2(CCN2)C1]

In some embodiments, Cy² is

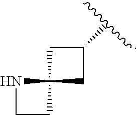 [SMILES: *[C@H]1C[C@@]2(CCN2)C1]

In some embodiments, Cy² is

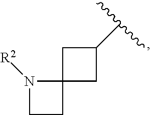

wherein R² is as defined in embodiments and classes and subclasses herein. In some embodiments, Cy² is

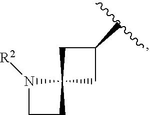

wherein R² is as defined in embodiments and classes and subclasses herein. In some embodiments, Cy² is

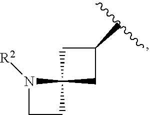

wherein R² is as defined in embodiments and classes and subclasses herein. In some embodiments, Cy² is

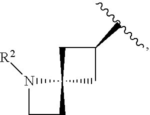

wherein R² is as defined in embodiments and classes and subclasses herein. In some embodiments, Cy² is

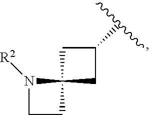

wherein R² is as defined in embodiments and classes and subclasses herein.

In some embodiments, Cy² is

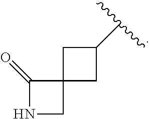 [SMILES: *C1CC2(CNC2=O)C1]

In some embodiments, Cy² is

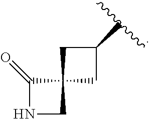 [SMILES: *[C@H]1C[C@@]2(CNC2=O)C1]

In some embodiments, Cy² is

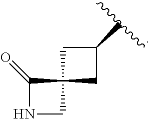 [SMILES: *[C@H]1C[C@]2(CNC2=O)C1]

In some embodiments, Cy² is

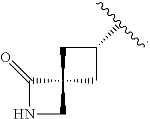 [SMILES: *[C@H]1C[C@]2(CNC2=O)C1]

In some embodiments, Cy² is

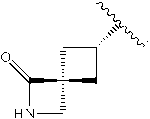 [SMILES: *[C@H]1C[C@@]2(CNC2=O)C1]

In some embodiments, Cy² is

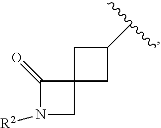

wherein R² is as defined in embodiments and classes and subclasses herein. In some embodiments, Cy² is

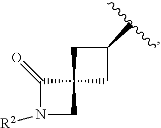

wherein R² is as defined in embodiments and classes and subclasses herein. In some embodiments, Cy² is

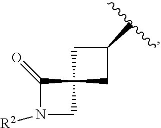

wherein R² is as defined in embodiments and classes and subclasses herein. In some embodiments, Cy² is

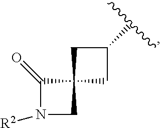

wherein R² is as defined in embodiments and classes and subclasses herein. In some embodiments, Cy² is

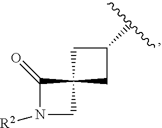

wherein R² is as defined in embodiments and classes and subclasses herein.

In some embodiments, Cy² is

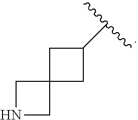 [SMILES: *C1CC2(CNC2)C1]

In some embodiments, Cy² is

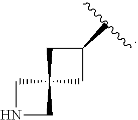

In some embodiments, Cy² is

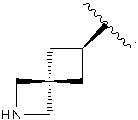

In some embodiments, Cy² is

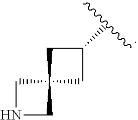

In some embodiments, Cy² is

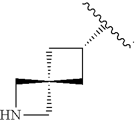

In some embodiments, Cy² is

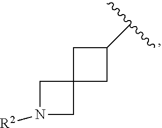

wherein R² is as defined in embodiments and classes and subclasses herein. In some embodiments, Cy² is

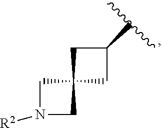

wherein R² is as defined in embodiments and classes and subclasses herein. In some embodiments, Cy² is

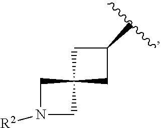

wherein R² is as defined in embodiments and classes and subclasses herein. In some embodiments, Cy² is

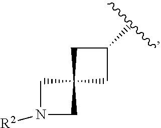

wherein R² is as defined in embodiments and classes and subclasses herein. In some embodiments, Cy² is

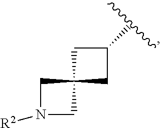

wherein R² is as defined in embodiments and classes and subclasses herein.

In some embodiments, Cy² is a 5-6 membered monocyclic heteroaryl ring having 1-4 heteroatoms independently selected from nitrogen, oxygen, and sulfur, wherein Cy² is substituted with m instances of R². In some embodiments, Cy² is a 5-6 membered monocyclic heteroaryl ring having 1-2 heteroatoms independently selected from nitrogen, oxygen, and sulfur, wherein Cy² is substituted with m instances of R². In some embodiments, Cy² is pyridyl, pyrimidinyl, pyridazinyl, triazinyl, or tetrazinyl. In some embodiments, Cy² is

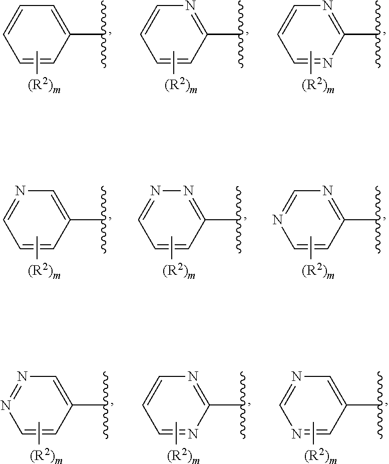

wherein each R² is as defined in embodiments and classes and subclasses herein. In some embodiments, Cy² is

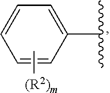

wherein each R² is as defined in embodiments and classes and subclasses herein. In some embodiments, Cy² is

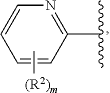

wherein each R² is as defined in embodiments and classes and subclasses herein. In some embodiments, Cy² is

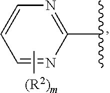

wherein each R² is as defined in embodiments and classes and subclasses herein. In some embodiments, Cy² is

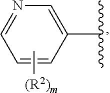

wherein each R² is as defined in embodiments and classes and subclasses herein. In some embodiments, Cy² is

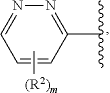

wherein each R² is as defined in embodiments and classes and subclasses herein. In some embodiments, Cy² is

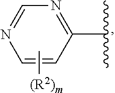

wherein each R² is as defined in embodiments and classes and subclasses herein. In some embodiments. Cy² is

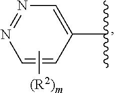

wherein each R² is as defined in embodiments and classes and subclasses herein. In some embodiments, Cy² is

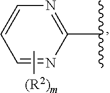

wherein each R² is as defined in embodiments and classes and subclasses herein. In some embodiments, Cy² is

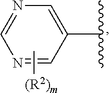

wherein each R² is as defined in embodiments and classes and subclasses herein.

In some embodiments, Cy² is a 3-7 membered saturated or partially unsaturated monocyclic heterocyclic ring having 1-2 heteroatoms independently selected from nitrogen, oxygen, and sulfur; wherein Cy² is substituted with m instances of R². In some embodiments, Cy² is aziridinyl, oxiranyl, azetidinyl, oxetanyl, pyrrolidinyl, piperidinyl, piperazinyl, tetrahydrofuranyl, tetrahydropyranyl, dioxanyl, morpholinyl, tetrahydrothiofuranyl, tetrahydrothiopyranyl, thiomorpholinyl, azepanyl, homomorpholinyl, and homothiomorpholinyl. In some embodiments, Cy² is azetidinyl, pyrrolidinyl, or piperidinyl. In some embodiments, Cy² is

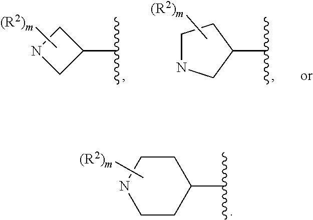

In some embodiments, Cy² is selected from the groups depicted in the compounds in Table 1.

As defined generally above, Q is L^{Q}, wherein L^{Q} is as defined in embodiments and classes and subclasses herein.

In some embodiments, Q is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a covalent bond. In some embodiments, Q is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a C₁₋₄ bivalent saturated or unsaturated, straight, or branched hydrocarbon chain.

In some embodiments, Q is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, Q is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, Q is a C₁₋₂ bivalent saturated or unsaturated hydrocarbon chain.

In some embodiments, Q is —C(O)N(R)—, —C(O)N(R)CH₂—, —N(R)—, —CH₂C(O)N(R)—, —N(R)C(O)N(R)—, or a covalent bond. In some embodiments, Q is —C(O)N(H)—, —C(O)N(H)CH₂—, —N(H)—, —CH₂C(O)N(H)—, —N(H)C(O)N(H)—, or a covalent bond. In some embodiments, Q is —C(O)N(H)—, —C(O)N(H)CH₂—, or a covalent bond. In some embodiments, Q is —C(O)N(H)— or —C(O)N(H)CH₂—. In some embodiments, Q is —C(O)N(H)—. In some embodiments, Q is —C(O)N(H)CH₂—. In some embodiments, Q is —N(H)—. In some embodiments, Q is —CH₂C(O)N(H)—. In some embodiments, Q is —N(H)C(O)N(H)—. In some embodiments, Q is a covalent bond.

In some embodiments, Q is selected from the groups depicted in the compounds in Table 1.

As defined generally above, T is a bivalent C₁₋₃ aliphatic chain substituted with q instances of R^{T}. In some embodiments, T is a bivalent C₂₋₃ aliphatic chain substituted with q instances of R^{T}. In some embodiments, T is a bivalent C₁₋₂ aliphatic chain substituted with q instances of R^{T}. In some embodiments, T is a bivalent C₁ aliphatic chain substituted with q instances of R^{T}. In some embodiments, T is a bivalent C₂ aliphatic chain substituted with q instances of R^{T}. In some embodiments, T is a bivalent C₃ aliphatic chain substituted with q instances of R^{T}.

In some embodiments, T is

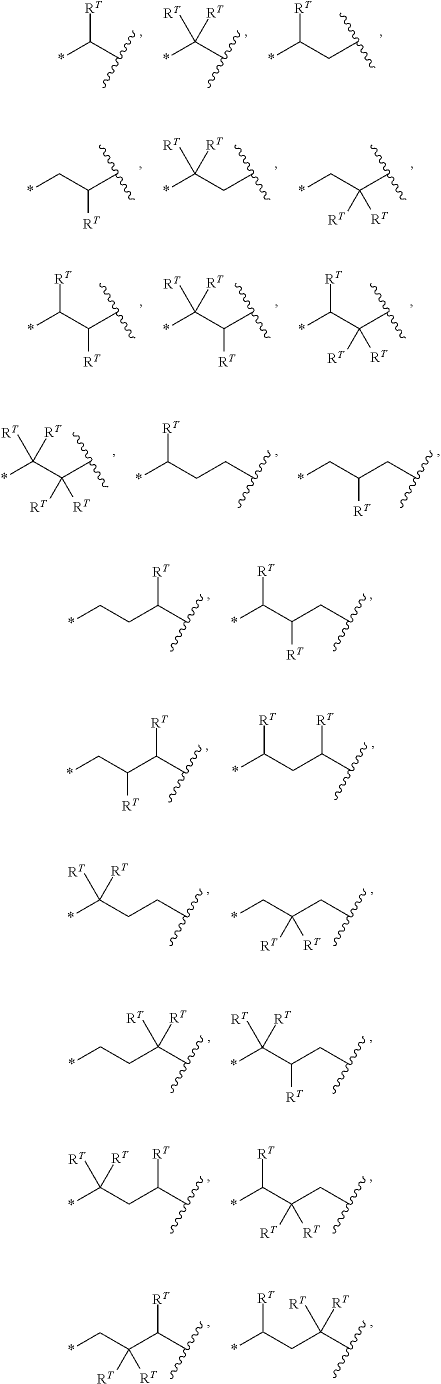

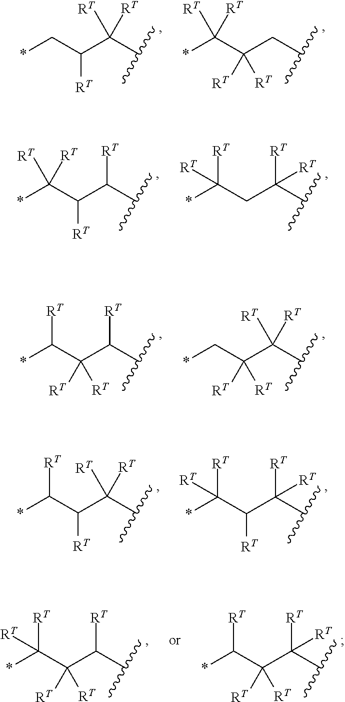

wherein [symbol] represents a bond to Q and

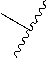

represents a bond to Cy¹, and wherein R^{T} is as defined in embodiments and classes and subclasses herein.

In some embodiments, T is

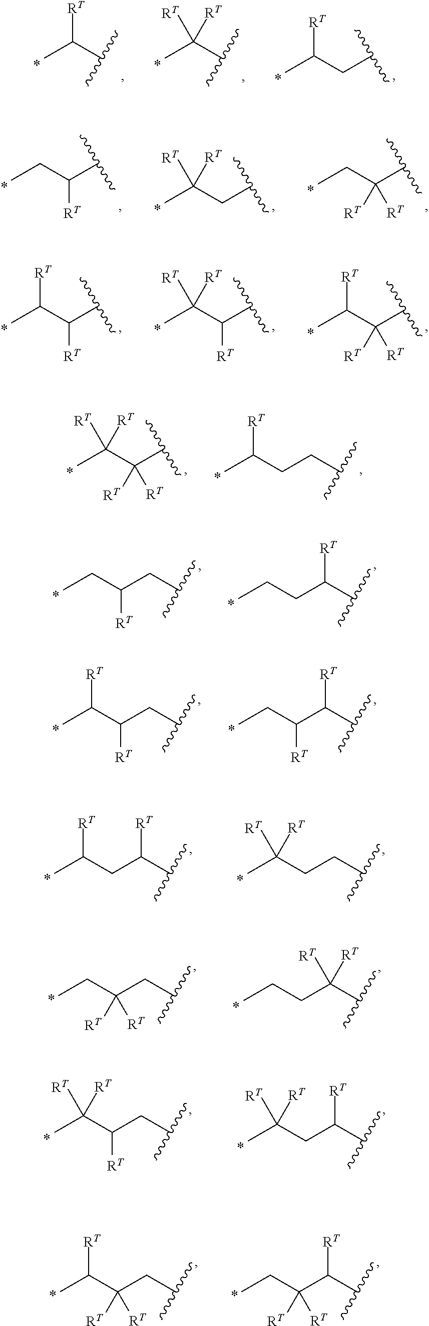

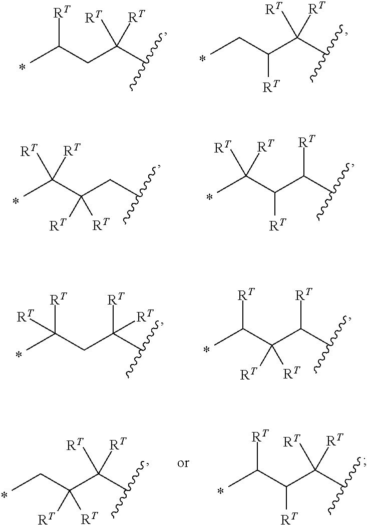

wherein [symbol] represents a bond to Q and

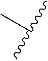

represents a bond to Cy¹, and wherein R^{T} is as defined in embodiments and classes and subclasses herein.

In some embodiments, T is

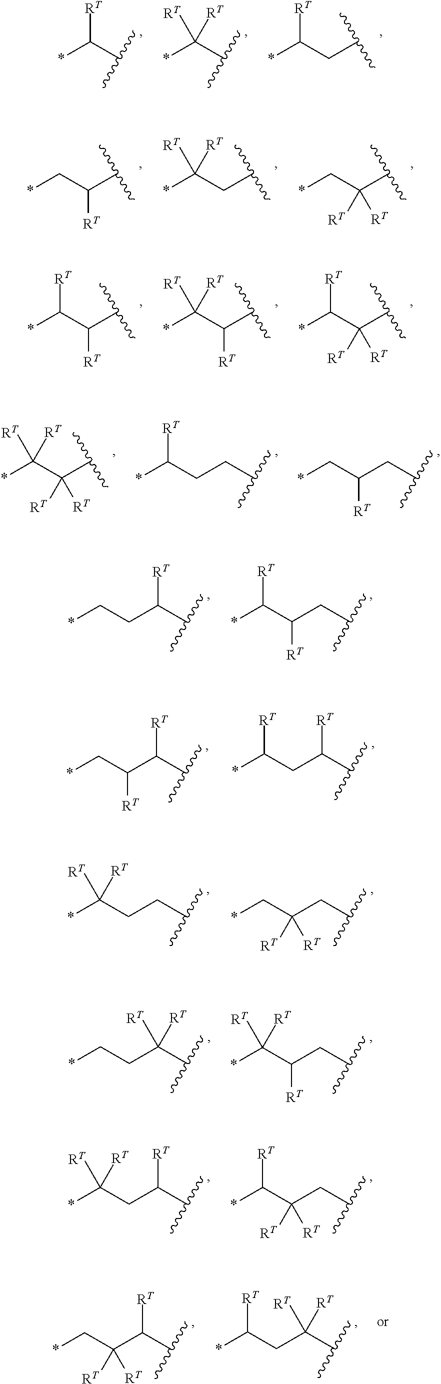

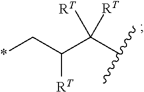

wherein [symbol] represents a bond to Q and

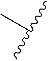

represents a bond to Cy¹, and wherein R^{T} is as defined in embodiments and classes and subclasses herein.

In some embodiments, T is

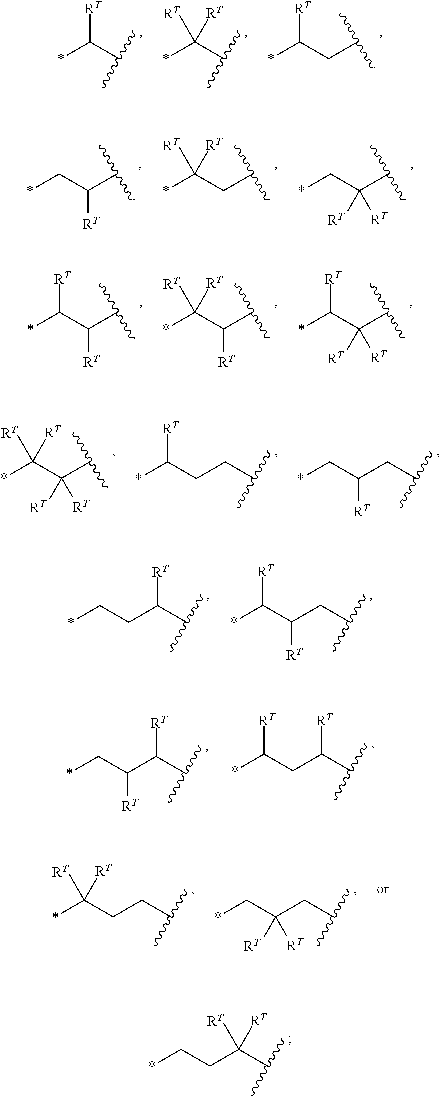

wherein [symbol] represents a bond to Q and

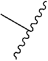

represents a bond to Cy¹, and wherein R^{T} is as defined in embodiments and classes and subclasses herein.

In some embodiments, T is

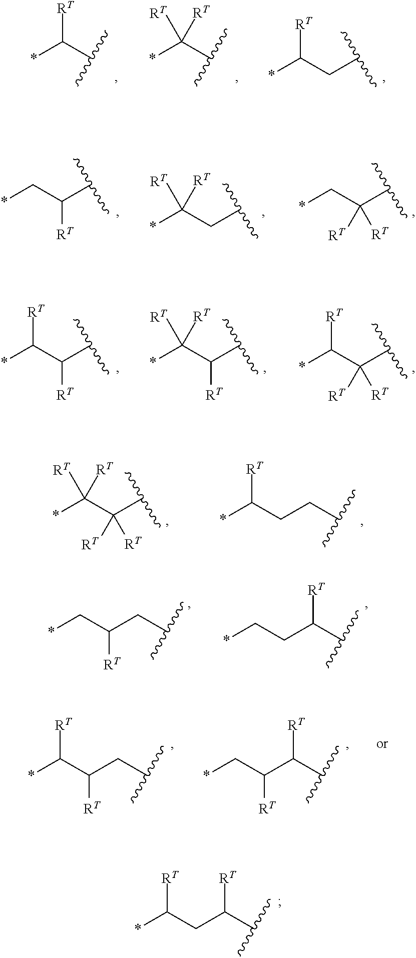

wherein [symbol] represents a bond to Q and

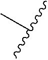

represents a bond to Cy¹, and wherein R^{T} is as defined in embodiments and classes and subclasses herein.

In some embodiments, T is

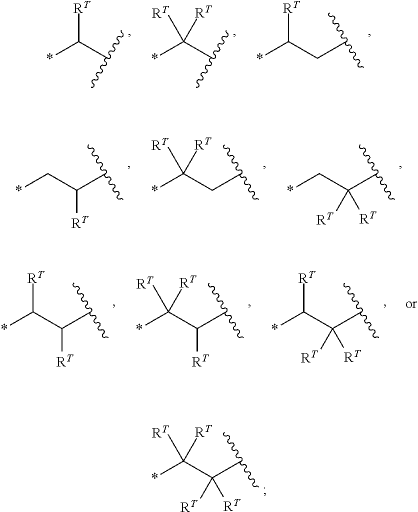

wherein [symbol] represents a bond to Q and

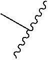

represents a bond to Cy¹, and wherein R^{T} is as defined in embodiments and classes and subclasses herein.

In some embodiments, T is

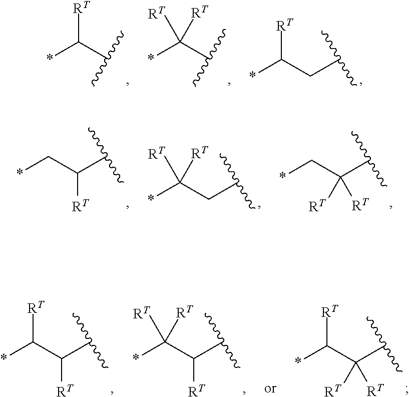

wherein [symbol] represents a bond to Q and

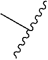

represents a bond to Cy¹, and wherein R^{T} is as defined in embodiments and classes and subclasses herein.

In some embodiments, T is

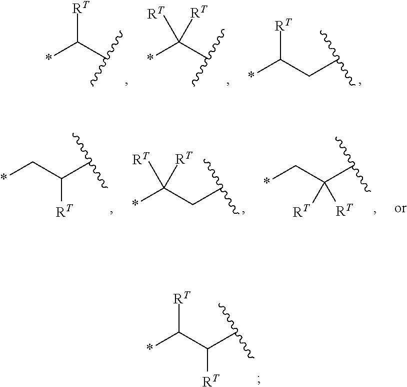

wherein [symbol] represents a bond to Q and

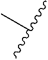

represents a bond to Cy¹, and wherein R^{T} is as defined in embodiments and classes and subclasses herein.

In some embodiments, T is

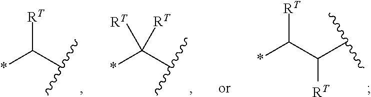

wherein [symbol] represents a bond to Q and

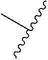

represents a bond to Cy¹, and wherein R^{T} is as defined in embodiments and classes and subclasses herein.

In some embodiments, T is

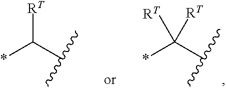

wherein [symbol] represents a bond to Q and

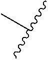

represents a bond to Cy¹, and wherein R^{T} is as defined in embodiments and classes and subclasses herein.

In some embodiments, T is

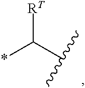

wherein [symbol] represents a bond to Q and

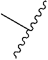

represents a bond to Cy¹, and wherein R^{T} is as defined in embodiments and classes and subclasses herein. In some embodiments, T is

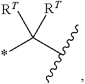

wherein [symbol] represents a bond to Q and

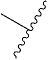

represents a bond to Cy¹, and wherein R^{T} is as defined in embodiments and classes and subclasses herein. In some embodiments, T is

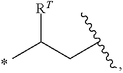

wherein [symbol] represents a bond to Q and

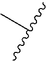

represents a bond to Cy¹, and wherein R^{T} is as defined in embodiments and classes and subclasses herein. In some embodiments, T is

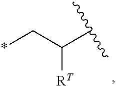

wherein [symbol] represents a bond to Q and

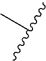

represents a bond to Cy¹, and wherein R^{T} is as defined in embodiments and classes and subclasses herein. In some embodiments, T is

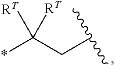

wherein [symbol] represents a bond to Q and

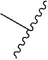

represents a bond to Cy¹, and wherein R^{T} is as defined in embodiments and classes and subclasses herein. In some embodiments, T is

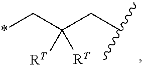

wherein [symbol] represents a bond to Q and

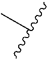

represents a bond to Cy¹, and wherein R^{T} is as defined in embodiments and classes and subclasses herein. In some embodiments, T is

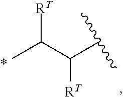

wherein [symbol] represents a bond to Q and

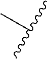

represents a bond to Cy¹, and wherein R^{T} is as defined in embodiments and classes and subclasses herein. In some embodiments, T is

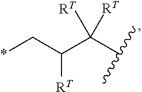

wherein [symbol] represents a bond to Q and

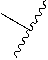

represents a bond to Cy¹, and wherein R^{T} is as defined in embodiments and classes and subclasses herein. In some embodiments, T is

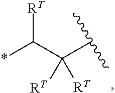

wherein [symbol] represents a bond to Q and

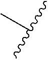

represents a bond to Cy¹, and wherein R^{T} is as defined in embodiments and classes and subclasses herein. In some embodiments, T is

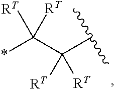

wherein [symbol] represents a bond to Q and

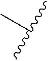

represents a bond to Cy¹, and wherein R^{T} is as defined in embodiments and classes and subclasses herein. In some embodiments, T is

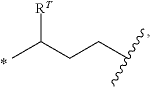

wherein [symbol] represents a bond to Q and

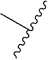

represents a bond to Cy¹, and wherein R^{T} is as defined in embodiments and classes and subclasses herein. In some embodiments, T is

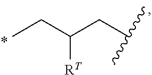

wherein [symbol] represents a bond to Q and

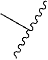

represents a bond to Cy¹, and wherein R^{T} is as defined in embodiments and classes and subclasses herein. In some embodiments, T is

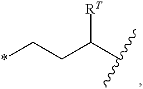

wherein [symbol] represents a bond to Q and

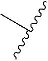

represents a bond to Cy¹, and wherein R^{T} is as defined in embodiments and classes and subclasses herein. In some embodiments, T is

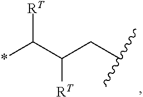

wherein [symbol] represents a bond to Q and

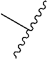

represents a bond to Cy¹, and wherein R^{T} is as defined in embodiments and classes and subclasses herein. In some embodiments, T is

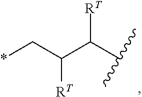

wherein [symbol] represents a bond to Q and

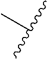

represents a bond to Cy¹, and wherein R^{T} is as defined in embodiments and classes and subclasses herein. In some embodiments, T is

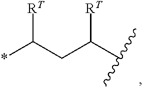

wherein [symbol] represents a bond to Q and

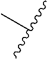

represents a bond to Cy¹, and wherein R^{T} is as defined in embodiments and classes and subclasses herein. In some embodiments, T is

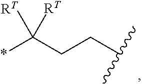

wherein [symbol] represents a bond to Q and

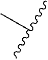

represents a bond to Cy¹, and wherein R^{T} is as defined in embodiments and classes and subclasses herein. In some embodiments, T is

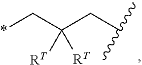

wherein [symbol] represents a bond to Q and

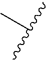

represents a bond to Cy¹, and wherein R^{T} is as defined in embodiments and classes and subclasses herein. In some embodiments, T is,

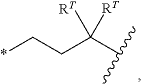

wherein [symbol] represents a bond to Q and

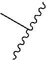

represents a bond to Cy¹, and wherein R^{T} is as defined in embodiments and classes and subclasses herein. In some embodiments, T is

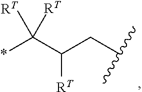

wherein [symbol] represents a bond to Q and

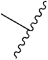

represents a bond to Cy¹, and wherein R^{T} is as defined in embodiments and classes and subclasses herein. In some embodiments, T is

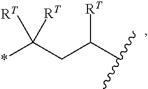

wherein [symbol] represents a bond to Q and

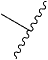

represents a bond to Cy¹, and wherein R^{T} is as defined in embodiments and classes and subclasses herein. In some embodiments, T is

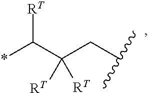

wherein [symbol] represents a bond to Q and

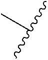

represents a bond to Cy¹, and wherein R^{T} is as defined in embodiments and classes and subclasses herein. In some embodiments, T is

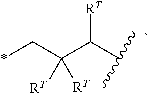

wherein [symbol] represents a bond to Q and

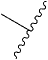

represents a bond to Cy¹, and wherein R^{T} is as defined in embodiments and classes and subclasses herein. In some embodiments, T is

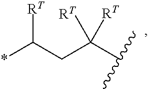

wherein [symbol] represents a bond to Q and

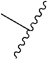

represents a bond to Cy¹, and wherein R^{T} is as defined in embodiments and classes and subclasses herein. In some embodiments, T is

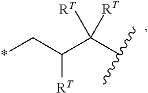

wherein [symbol] represents a bond to Q and

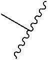

represents a bond to Cy¹, and wherein R^{T} is as defined in embodiments and classes and subclasses herein. In some embodiments, T is

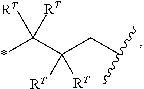

wherein [symbol] represents a bond to Q and

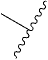

represents a bond to Cy¹, and wherein R^{T} is as defined in embodiments and classes and subclasses herein. In some embodiments, T is

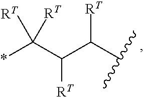

wherein [symbol] represents a bond to Q and

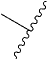

represents a bond to Cy¹, and wherein R^{T} is as defined in embodiments and classes and subclasses herein. In some embodiments, T is

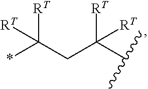

wherein [symbol] represents a bond to Q and

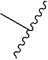

represents a bond to Cy¹, and wherein R^{T} is as defined in embodiments and classes and subclasses herein. In some embodiments, T is

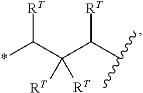

wherein [symbol] represents a bond to Q and

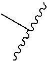

represents a bond to Cy¹, and wherein R^{T} is as defined in embodiments and classes and subclasses herein. In some embodiments, T is

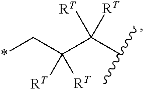

wherein [symbol] represents a bond to Q and

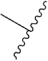

represents a bond to Cy¹, and wherein R^{T} is as defined in embodiments and classes and subclasses herein. In some embodiments, T is

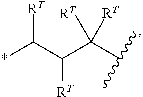

wherein [symbol] represents a bond to Q and

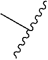

represents a bond to Cy¹, and wherein R^{T} is as defined in embodiments and classes and subclasses herein. In some embodiments, T is

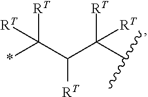

wherein [symbol] represents a bond to Q and

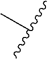

represents a bond to Cy¹, and wherein R^{T} is as defined in embodiments and classes and subclasses herein. In some embodiments, T is

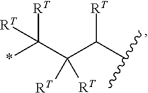

wherein [symbol] represents a bond to Q and

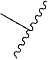

represents a bond to Cy¹, and wherein R^{T} is as defined in embodiments and classes and subclasses herein. In some embodiments, T is

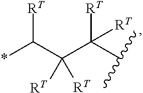

wherein [symbol] represents a bond to Q and

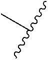

represents a bond to Cy¹, and wherein R^{T} is as defined in embodiments and classes and subclasses herein.

In some embodiments, T is

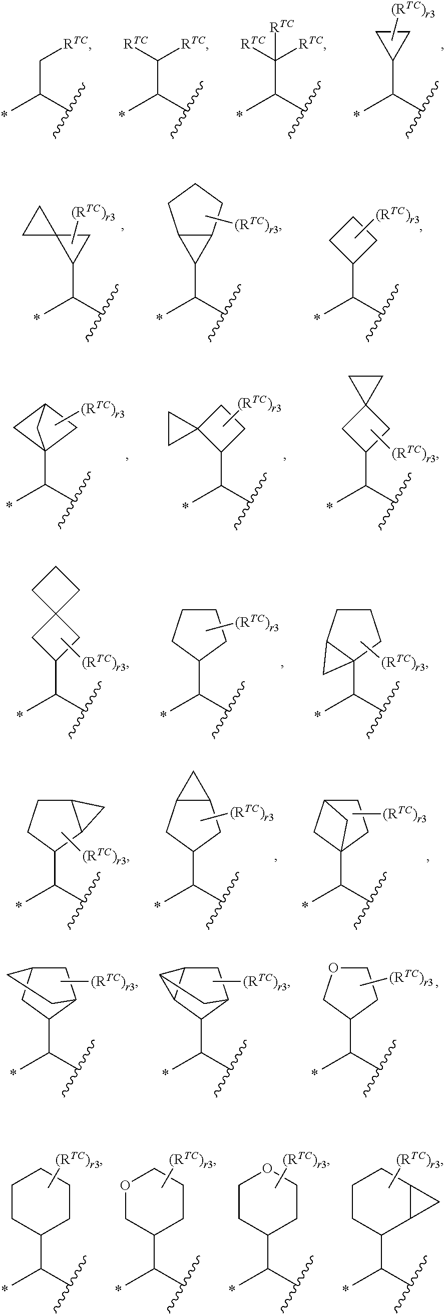

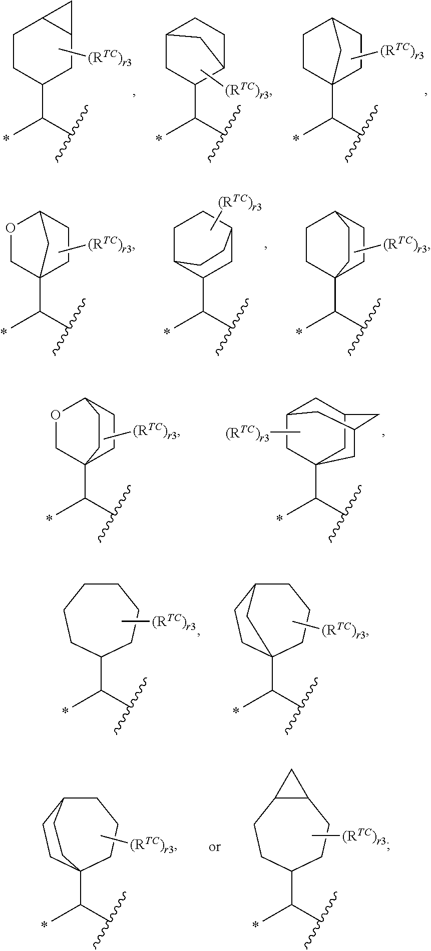

wherein [symbol] represents a bond to Q and

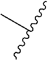

represents a bond to Cy¹, and wherein R^{TC} and r³ are as defined in embodiments and Classes and subclasses herein.

In some embodiments, T is

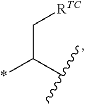

wherein [symbol] represents a bond to Q and

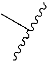

represents a bond to Cy¹, and wherein R^{TC} is as defined in embodiments and classes and subclasses herein. In some embodiments, T is

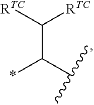

wherein [symbol] represents a bond to Q and

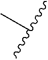

represents a bond to Cy¹, and wherein R^{TC} is as defined in embodiments and classes and subclasses herein. In some embodiments, T is

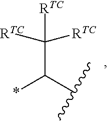

wherein [symbol] represents a bond to Q and

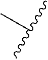

represents a bond to Cy¹, and wherein R^{TC} is as defined in embodiments and classes and subclasses herein. In some embodiments, T is

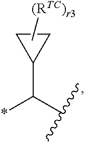

wherein [symbol] represents a bond to Q and

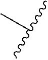

represents a bond to Cy¹, and wherein R^{TC} an r³ are as defined in embodiments and classes and subclasses herein. In some embodiments, T is

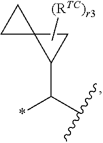

wherein [symbol] represents a bond to Q and

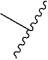

represents a bond to Cy¹, and wherein R^{TC} an r³ are as defined in embodiments and classes and subclasses herein. In some embodiments, T is

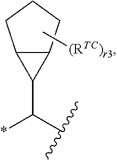

wherein [symbol] represents a bond to Q and

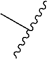

represents a bond to Cy¹, and wherein R^{T}c and r³ are as defined in embodiments and classes and subclasses herein. In some embodiments, T is

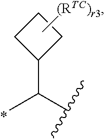

wherein [symbol] represents a bond to Q and

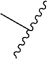

represents a bond to Cy¹, and wherein R^{TC} and r³ are as defined in embodiments and classes and subclasses herein. In some embodiments, T is

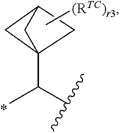

wherein [symbol] represents a bond to Q and

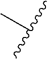

represents a bond to Cy¹, and wherein R^{TC} and r³ are as defined in embodiments and classes and subclasses herein. In some embodiments, T is,

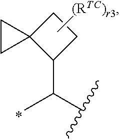

wherein [symbol] represents a bond to Q and

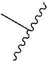

represents a bond to Cy¹, and wherein R^{TC} and r³ are as defined in embodiments and classes and subclasses herein. In some embodiments, T is

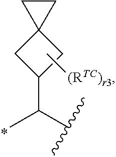

wherein [symbol] represents a bond to Q and

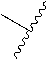

represents a bond to Cy¹, and wherein R^{TC} and r³ are as defined in embodiments and classes and subclasses herein. In some embodiments, T is

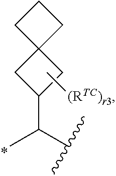

wherein [symbol] represents a bond to Q and

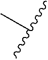

represents a bond to Cy¹, and wherein R^{TC} and r³ are as defined in embodiments and classes and subclasses herein. In some embodiments, T is

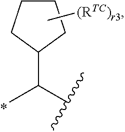

wherein [symbol] represents a bond to Q and

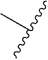

represents a bond to Cy¹, and wherein R^{TC} and r³ are as defined in embodiments and classes and subclasses herein. In some embodiments, T is

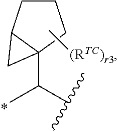

wherein [symbol] represents a bond to Q and

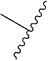

represents a bond to Cy¹, and wherein R^{TC} and r³ are as defined in embodiments and classes and subclasses herein. In some embodiments, T is

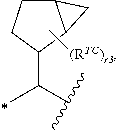

wherein [symbol] represents a bond to Q and

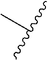

represents a bond to Cy¹, and wherein R^{TC} and r³ are as defined in embodiments and classes and subclasses herein. In some embodiments, T is

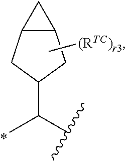

wherein [symbol] represents a bond to Q and

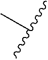

represents a bond to Cy¹, and wherein R^{TC} and r³ are as defined in embodiments and classes and subclasses herein. In some embodiments, T is

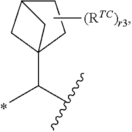

wherein [symbol] represents a bond to Q and

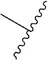

represents a bond to Cy¹, and wherein R^{TC} and r³ are as defined in embodiments and classes and subclasses herein. In some embodiments, T is

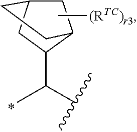

wherein [symbol] represents a bond to Q and

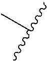

represents a bond to Cy¹, and wherein R^{TC} and r³ are as defined in embodiments and classes and subclasses herein. In some embodiments, T is

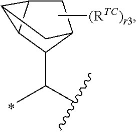

wherein [symbol] represents a bond to Q and

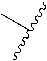

represents a bond to Cy¹, and wherein R^{TC} and r³ are as defined in embodiments and classes and subclasses herein. In some embodiments, T is

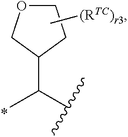

wherein [symbol] represents a bond to Q and

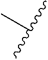

represents a bond to Cy¹, and wherein R^{TC} and r³ are as defined in embodiments and classes and subclasses herein. In some embodiments, T is

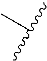

wherein [symbol] represents a bond to Q and

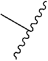

represents a bond to Cy¹, and wherein R^{TC} and r³ are as defined in embodiments and classes and subclasses herein. In some embodiments, T is

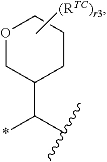

wherein [symbol] represents a bond to Q and

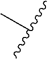

represents a bond to Cy¹, and wherein R^{TC} and r³ are as defined in embodiments and classes and subclasses herein. In some embodiments, T is

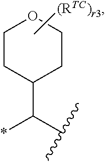

wherein [symbol] represents a bond to Q and

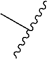

represents a bond to Cy¹, and wherein R^{TC} and r³ are as defined in embodiments and classes and subclasses herein. In some embodiments, T is

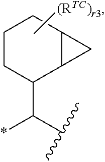

wherein [symbol] represents a bond to Q and

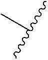

represents a bond to Cy¹, and wherein R^{TC} and r³ are as defined in embodiments and classes and subclasses here in. In some embodiments, T is

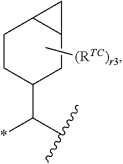

wherein [symbol] represents a bond to Q and

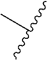

represents a bond to Cy¹, and wherein R^{TC} and r³ are as defined in embodiments and classes and subclasses herein. In some embodiments, T is

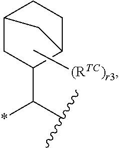

wherein [symbol] represents a bond to Q and

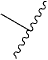

represents a bond to Cy¹, and wherein R^{TC} and r³ are as defined in embodiments and classes and subclasses herein. In some embodiments, T is

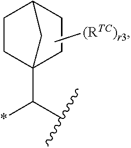

wherein [symbol] represents a bond to Q and

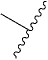

represents a bond to Cy¹, and wherein R^{TC} and r³ are as defined in embodiments and classes and subclasses herein. In some embodiments, T is

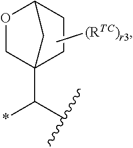

wherein [symbol] represents a bond to Q and

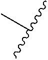

represents a bond to Cy¹, and wherein R^{TC} and r³ are as defined in embodiments and classes and subclasses herein. In some embodiments, T is

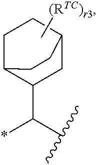

wherein [symbol] represents a bond to Q and

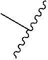

represents a bond to Cy¹, and wherein R^{TC} and r³ are as defined in embodiments and classes and subclasses herein. In some embodiments, T is

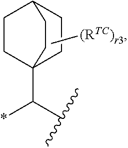

wherein [symbol] represents a bond to Q and

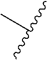

represents a bond to Cy¹, and wherein R^{TC} and r³ are as defined in embodiments and classes and subclasses herein. In some embodiments, T is

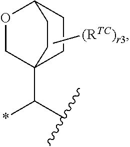

wherein [symbol] represents a bond to Q and

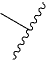

represents a bond to Cy¹, and wherein R^{TC} and r³ are as defined in embodiments and classes and subclasses herein. In some embodiments, T is

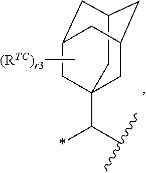

wherein [symbol] represents a bond to Q and

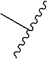

represents a bond to Cy¹, and wherein R^{TC} and r³ are as defined in embodiments and classes and subclasses herein. In some embodiments, T is

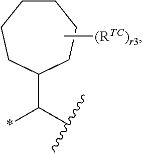

wherein [symbol] represents a bond to Q and

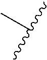

represents a bond to Cy¹, and wherein R^{TC} and r³ are as defined in embodiments and classes and subclasses herein. In some embodiments, T is

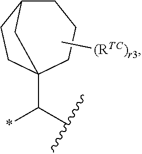

wherein [symbol] represents a bond to Q and

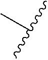

represents a bond to Cy¹, and wherein R^{TC} and r³ are as defined in embodiments and classes and subclasses herein. In some embodiments, T is

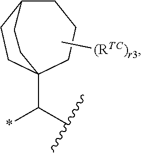

wherein [symbol] represents a bond to Q and

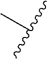

represents a bond to Cy¹, and wherein R^{TC} and r³ are as defined in embodiments and classes and subclasses herein. In some embodiments, T is

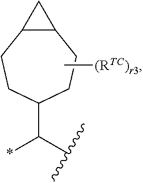

wherein [symbol] represents a bond to Q and

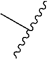

represents a bond to Cy¹, and wherein R^{TC} and r³ are as defined in embodiments and classes and subclasses herein.

In some embodiments, T is

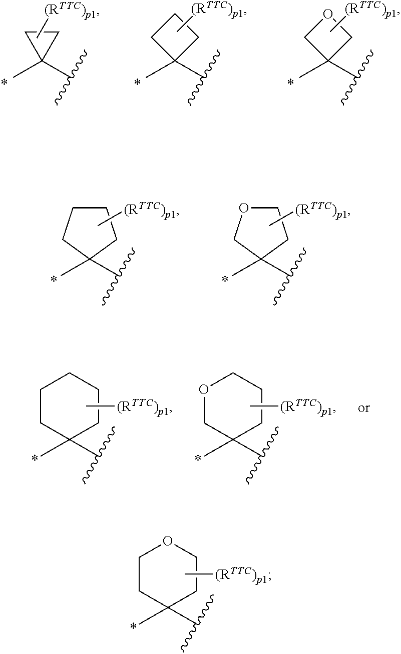

wherein [symbol] and represents a bond to Q and

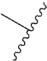

represents a bond to Cy¹, and wherein R^{TTC} and p¹ are as defined in embodiments and classes and subclasses herein.

In some embodiments, T is

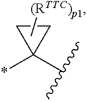

wherein [symbol] represents a bond to Q and

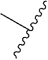

represents a bond to Cy¹, and wherein R^{TTC} and p¹ are as defined in embodiments and classes and subclasses herein. In some embodiments, T is

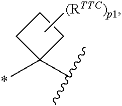

wherein [symbol] represents a bond to Q and

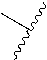

represents a bond to Cy¹, and wherein R^{TTC} and p¹ are as defined in embodiments and classes and subclasses herein. In some embodiments, T is

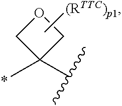

wherein [symbol] represents a bond to Q and

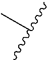

represents a bond to Cy¹, and wherein R^{TTC} and p¹ are as defined in embodiments and classes and subclasses herein. In some embodiments, T is

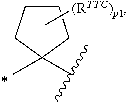

wherein [symbol] represents a bond to Q and

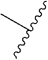

represents a bond to Cy¹, and wherein R^{TTC} and p¹ are as defined in embodiments and classes and subclasses herein. In some embodiments, T is

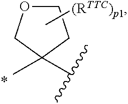

wherein [symbol] represents a bond to Q and

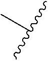

represents a bond to Cy¹, and wherein R^{TTC} and p¹ are as defined in embodiments and classes and subclasses herein. In some embodiments, T is

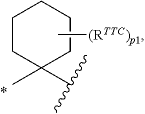

wherein [symbol] represents a bond to Q and

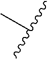

represents a bond to Cy¹, and wherein R^{TTC} and p¹ are defined in embodiments and classes and subclasses herein. In some embodiments, T is

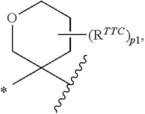

wherein [symbol] represents a bond to Q and

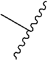

represents a bond to Cy¹, and wherein R^{TTC} and p¹ are as defined in embodiments and classes and subclasses herein. In some embodiments, T is

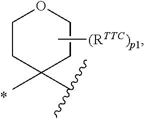

wherein [symbol] represents a bond to Q and

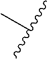

represents a bond to Cy¹, and wherein R^{TTC} and p¹ are as defined in embodiments and classes and subclasses herein.

In some embodiments, T is

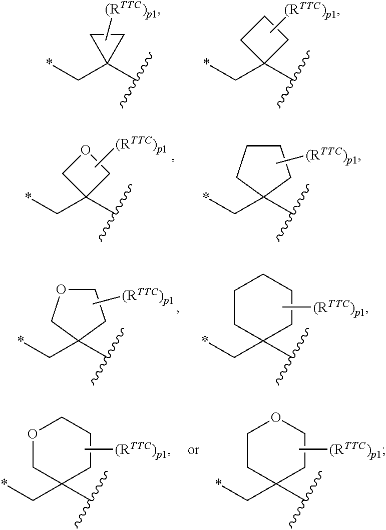

wherein [symbol] represents a bond to Q and

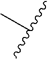

represents a bond to Cy¹, and wherein R^{TTC} and p¹ are as defined in embodiments and classes and subclasses herein.

In some embodiments, T is

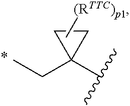

wherein [symbol] represents a bond to Q and

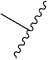

represents a bond to Cy¹, and wherein R^{TTC} and p¹ are as defined in embodiments and classes and subclasses herein. In some embodiments, T is

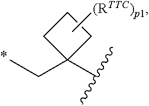

wherein [symbol] represents a bond to Q and

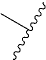

represents a bond to Cy¹, and wherein R^{TTC} and p¹ are as defined in embodiments and classes and subclasses herein. In some embodiments, T is

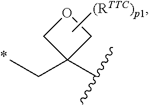

wherein [symbol] represents a bond to Q and

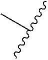

represents a bond to Cy¹, and wherein R^{TTC} and p¹ are as defined in embodiments and classes and subclasses herein. In some embodiments, T is

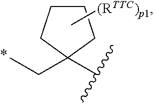

wherein [symbol] represents a bond to Q and

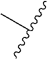

represents a bond to Cy¹, and wherein R^{TTC} and p¹ are as defined in embodiments and classes and subclasses herein. In some embodiments, T is

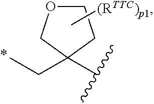

wherein [symbol] represents a bond to Q and

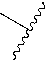

represents a bond to Cy¹, and wherein R^{TTC} and p¹ are as defined in embodiments and classes and subclasses herein. In some embodiments, T is

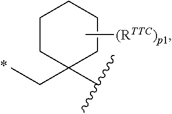

wherein [symbol] represents a bond to Q and

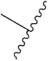

represents a bond to Cy¹, and wherein R^{TTC} and p¹ are as defined in embodiments and classes and subclasses herein. In some embodiments, T is

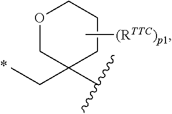

wherein [symbol] represents a bond to Q and

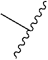

represents a bond to Cy¹, and wherein R^{TTC} and p¹ are as defined in embodiments and classes and subclasses herein. In some embodiments, T is

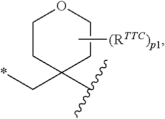

wherein [symbol] represents a bond to Q and

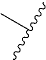

represents a bond to Cy¹, and wherein R^{TTC} and p¹ are as defined in embodiments and classes and subclasses herein.

In some embodiments, T is

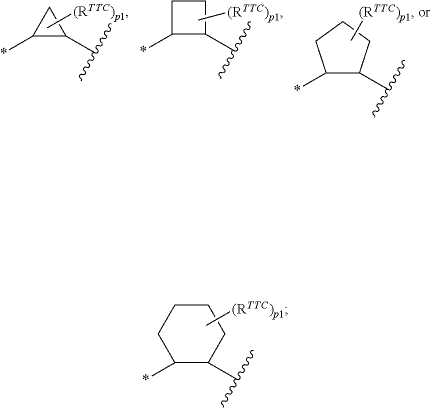

wherein [symbol] represents a bond to Q and

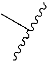

represents a bond to Cy¹, and wherein R^{TTC} and p¹ are as defined in embodiments and classes and subclasses herein.

In some embodiments, T is

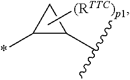

wherein [symbol] represents a bond to Q and

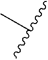

represents a bond to Cy¹, and wherein R^{TTC} and p¹ are as defined in embodiments and classes and subclasses herein. In some embodiments, T is

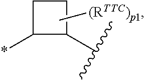

wherein [symbol] represents a bond to Q and

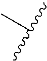

represents a bond to Cy¹, and wherein R^{TTC} and p¹ are as defined in embodiments and classes and subclasses herein. In some embodiments, T is

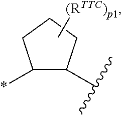

wherein [symbol] represents a bond to Q and

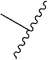

represents a bond to Cy¹, and wherein R^{TTC} and p¹ are as defined in embodiments and classes and subclasses herein. In some embodiments, T is

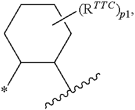

wherein [symbol] represents a bond to Q and

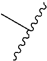

represents a bond to Cy¹, and wherein R^{TTC} and p¹ are as defined in embodiments and classes and subclasses herein. In some embodiments, T is

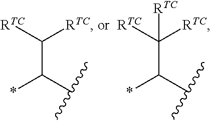

wherein each R^{TC} is as defined in embodiments and classes and subclasses herein. In some embodiments, T is

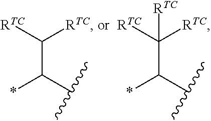

wherein each R^{TC} is as defined in embodiments and classes and subclasses herein. In some embodiments, T is

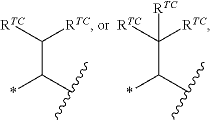

wherein each R^{TC} is independently deuterium, halogen, or an optionally substituted group selected from C₁₋₆ aliphatic. In some embodiments, T is

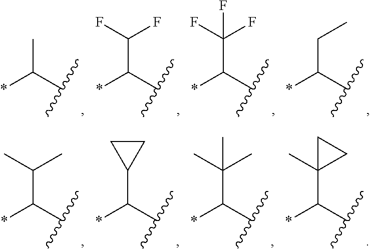

In some embodiments, T is

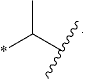 [SMILES: *C(*)C]

In some embodiments, T is

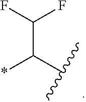 [SMILES: *C(*)C(F)F]

In some embodiments, T is

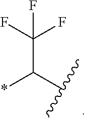 [SMILES: *C(*)C(F)(F)F]

In some embodiments, T is

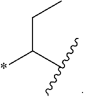 [SMILES: *C(*)CC]

In some embodiments, T is

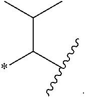 [SMILES: *C(*)C(C)C]

In some embodiments, T is

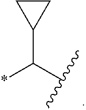 [SMILES: *C(*)C1CC1]

In some embodiments, T is

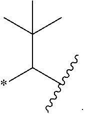 [SMILES: *C(*)C(C)(C)C]

In some embodiments, T is

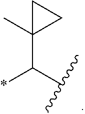 [SMILES: *C(*)C1(C)CC1]

In some embodiments, T is selected from the groups depicted in the compounds in Table 1.

As defined generally above, each R¹ is independently -L¹-R^{1A}; or two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C}; or one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}.

In some embodiments, each R¹ is independently -L¹-R^{1A}. In some embodiments, each R¹ is independently —R^{1A}.

In some embodiments, each R¹ is independently R^{A}. In some embodiments, each R¹ (i.e., -L¹-R^{1A} taken together) is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —S(O)(NCN)R, —S(NCN)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂; wherein R is as defined in embodiments and classes and subclasses herein.

In some embodiments, R¹ is oxo. In some embodiments, R¹ is deuterium. In some embodiments, each R¹ is independently halogen. In some embodiments, R¹ is —CN. In some embodiments, R¹ is —NO₂. In some embodiments, each R¹ is independently —OR, wherein R is as defined in embodiments and classes and subclasses herein. In some embodiments, R¹ is —SF₅. In some embodiments, each R¹ is independently —SR, wherein R is as defined in embodiments and classes and subclasses herein. In some embodiments, each R¹ is independently —NR₂, wherein R is as defined in embodiments and classes and subclasses herein. In some embodiments, each R¹ is independently —S(O)₂R, wherein R is as defined in embodiments and classes and subclasses herein. In some embodiments, each R¹ is independently —S(O)₂NR₂, wherein R is as defined in embodiments and classes and subclasses herein. In some embodiments, R¹ is —S(O)₂F. In some embodiments, each R¹ is independently —S(O)R, wherein R is as defined in embodiments and classes and subclasses herein. In some embodiments, each R¹ is independently —S(O)NR₂, wherein R is as defined in embodiments and classes and subclasses herein. In some embodiments, each R¹ is independently —S(O)(NR)R, wherein R is as defined in embodiments and classes and subclasses herein. In some embodiments, each R¹ is independently —S(O)(NCN)R, wherein R is as defined in embodiments and classes and subclasses herein. In some embodiments, each R¹ is independently —S(NCN)R, wherein R is as defined in embodiments and classes and subclasses herein. In some embodiments, each R¹ is independently —C(O)R, wherein R is as defined in embodiments and classes and subclasses herein. In some embodiments, each R¹ is independently —C(O)OR, wherein R is as defined in embodiments and classes and subclasses herein. In some embodiments, each R¹ is independently —C(O)NR₂, wherein R is as defined in embodiments and classes and subclasses herein. In some embodiments, each R¹ is independently —C(O)N(R)OR, wherein R is as defined in embodiments and classes and subclasses herein. In some embodiments, each R¹ is independently —OC(O)R, wherein R is as defined in embodiments and classes and subclasses herein. In some embodiments, each R¹ is independently —OC(O)NR₂, wherein R is as defined in embodiments and classes and subclasses herein. In some embodiments, each R¹ is independently —N(R)C(O)OR, wherein R is as defined in embodiments and classes and subclasses herein. In some embodiments, each R¹ is independently —N(R)C(O)R, wherein R is as defined in embodiments and classes and subclasses herein. In some embodiments, each R¹ is independently —N(R)C(O)NR₂, wherein R is as defined in embodiments and classes and subclasses herein. In some embodiments, each R¹ is independently —N(R)C(NR)NR₂, wherein R is as defined in embodiments and classes and subclasses herein. In some embodiments, each R¹ is independently —N(R)S(O)₂NR₂, wherein R is as defined in embodiments and classes and subclasses herein. In some embodiments, each R¹ is independently —N(R)S(O)₂R, wherein R is as defined in embodiments and classes and subclasses herein. In some embodiments, each R¹ is independently —P(O)R₂, wherein R is as defined in embodiments and classes and subclasses herein. In some embodiments, each R¹ is independently —P(O)(R)OR, wherein R is as defined in embodiments and classes and subclasses herein. In some embodiments, each R¹ is independently —B(OR)₂, wherein R is as defined in embodiments and classes and subclasses herein.

In some embodiments, each R¹ is independently R^{B} substituted by r instances of R^{1C}. In some embodiments, R¹ (i.e., -L¹-R^{1A} taken together) is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, each R¹ (i.e., -L¹-R^{1A} taken together) is independently halogen, —CN, —OR, or a C₁₋₆ aliphatic chain substituted with r¹ halogens. In some embodiments, each R¹ is independently halogen, —CN, —OR, or a C₁₋₆ aliphatic chain substituted with 0-5 halogens. In some embodiments, each R¹ is independently halogen, —CN, —O—(C₁₋₆ aliphatic chain substituted with 0-5 halogens), or a C₁₋₆ aliphatic chain substituted with 0-5 halogens. In some embodiments, each R¹ is independently halogen or a C₁₋₆ aliphatic chain substituted with 0-5 halogens. In some embodiments, each R¹ is independently halogen or a C₁₋₆ aliphatic chain substituted with 0-4 halogens. In some embodiments, each R¹ is independently halogen or a C₁₋₆ aliphatic chain substituted with 0-3 halogens. In some embodiments, each R¹ is independently halogen or a C₁₋₃ aliphatic chain substituted with 0-3 halogens. In some embodiments, each R¹ is independently halogen or a C₁₋₃ aliphatic chain substituted with 0-2 halogens.

In some embodiments, each R¹ is independently a halogen selected from Br, Cl, and F. In some embodiments, each R¹ is independently a halogen selected from Cl and F. In some embodiments, R¹ is Cl. In some embodiments, R¹ is F.

In some embodiments, at least one R¹ is halogen. In some embodiments, at least two R¹ are halogen. In some embodiments, at least three R¹ are halogen. In some embodiments, one instance of R¹ is Cl. In some embodiments, two instances of R¹ are Cl. In some embodiments, one instance of R¹ is F. In some embodiments, two instances of R¹ are F. In some embodiments, one instance of R¹ is Cl, and one instance of R¹ is F. In some embodiments, two instances of R¹ are Cl, and one instance of R¹ is F. In some embodiments, one instance of R¹ is Cl, and two instances of R¹ are F.

In some embodiments, each R¹ is independently a C₁₋₆ aliphatic chain substituted with 0-5 halogens. In some embodiments, each R¹ is independently a C₁₋₆ aliphatic chain substituted with 0-4 halogens. In some embodiments, each R¹ is independently a C₁₋₆ aliphatic chain substituted with 0-3 halogens. In some embodiments, each R¹ is independently a C₁₋₃ aliphatic chain substituted with 0-3 halogens. In some embodiments, each R¹ is independently a C₁₋₃ aliphatic chain substituted with 0-2 halogens.

In some embodiments, at least one R¹ is C₁₋₃ aliphatic optionally substituted with 1-3 halogen. In some embodiments, at least one R¹ is —O—C₁₋₃ aliphatic optionally substituted with 1-3 halogen.

In some embodiments, each R¹ (i.e., -L¹-R^{1A} taken together) is independently halogen, —OH, —OCH₃, or C₁₋₃ aliphatic optionally substituted with 1-3 halogen. In some embodiments, each R¹ is independently fluorine, chlorine, —OCH₃, or —CH₃. In some embodiments, R¹ is —OH. In some embodiments, R¹ is —CH₃. In some embodiments, R¹ is —OCH₃. In some embodiments, R¹ is —CF₃. In some embodiments, R¹ is —CHF₂.

In some embodiments, two instances of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p² instances of R^{11C}; wherein p² and R^{11C} are as defined in embodiments and classes and subclasses herein.

In some embodiments, one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; wherein p⁴ and R^{T1C} are as defined in embodiments and classes and subclasses herein.

In some embodiments, R¹ is selected from the groups depicted in the compounds in Table 1.

As defined generally above, each R² is independently -L²-R^{2A}; or two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C}.

In some embodiments, each R² is independently -L²-R^{2A}. In some embodiments each R² is independently R^{2A}.

In some embodiments, R² (i.e., -L²-R^{2A} taken together) is —N(R)—R^{2A}, wherein R and R^{2A} are as defined in embodiments and classes and subclasses herein. In some embodiments, R² (i.e., -L²-R^{2A} taken together) is —NH—R^{2A}, wherein R^{2A} is as defined in embodiments and classes and subclasses herein. In some embodiments, R² (i.e., -L²-R^{2A} taken together) is —CH(R^{L})—R^{2A}, wherein R and R^{2A} are as defined in embodiments and classes and subclasses herein. In some embodiments, R² (i.e., -L²-R^{2A} taken together) is —CH₂—R^{2A}, wherein R^{2A} is as defined in embodiments and classes and subclasses herein. In some embodiments, R² (i.e., -L²-R^{2A} taken together) is —N(R)C(O)—R^{2A}, wherein R and R^{2A} are as defined in embodiments and classes and subclasses herein. In some embodiments, R² (i.e., -L²-R^{2A} taken together) is —NHC(O)—R^{2A}, wherein R^{2A} is as defined in embodiments and classes and subclasses herein. In some embodiments, R² (i.e., -L²-R^{2A} taken together) is —N(R)C(O)CH(R^{L})—R^{2A}, wherein R and R^{2A} are as defined in embodiments and classes and subclasses herein. In some embodiments, R² (i.e., -L²-R^{2A} taken together) is —NHC(O)CH₂—R^{2A}, wherein R^{2A} is as defined in embodiments and classes and subclasses herein. In some embodiments, R² (i.e., -L²-R^{2A} taken together) is —N(R)C(O)N(R)—R^{2A}, wherein R and R^{2A} are as defined in embodiments and classes and subclasses herein. In some embodiments, R² (i.e., -L²-R^{2A} taken together) is —NHC(O)NH—R^{2A}, wherein R^{2A} is as defined in embodiments and classes and subclasses herein. In some embodiments, R² (i.e., -L²-R^{2A} taken together) is —N(R)C(O)CH(R^{L})O—R^{2A}, wherein R and R^{2A} are as defined in embodiments and classes and subclasses herein. In some embodiments, R² (i.e., -L²-R^{2A} taken together) is —NHC(O)CH₂O—R^{2A}, wherein R^{2A} is as defined in embodiments and classes and subclasses herein. In some embodiments, R² (i.e., -L²-R^{2A} taken together) is —CH(R^{L})N(R)—R^{2A}, wherein R and R^{2A} are as defined in embodiments and classes and subclasses herein. In some embodiments, R² (i.e., -L²-R^{2A} taken together) is —CH(R^{L})O—R^{2A}, wherein R and R^{2A} are as defined in embodiments and classes and subclasses herein.

In some embodiments, R² is —N(R)—R^{2A}, —N(R)C(O)—R^{2A}, —CH(R^{L})N(R)—R^{2A}, —N(R)C(O)CH(R^{L})—R^{2A}, —CH(R^{L})O—R^{2A}, —CH(R^{L})—R^{2A}, or —R^{2A}. In some embodiments, R² is —N(R)—R^{2A}, —N(R)C(O)—R^{2A}, —CH(R^{L})N(R)—R^{2A}, or —R^{2A}. In some embodiments, R² is —N(R)—R^{2A}, —N(R)C(O)—R^{2A}, or —R^{2A}. In some embodiments, R² is —N(R)C(O)—R^{2A} or —R^{2A}.

In some embodiments, R² is —N(H)—R^{2A}, —N(H)C(O)—R^{2A}, —CH₂N(H)—R^{2A}, —N(H)C(O)CH₂—R^{2A}, —CH₂O—R^{2A}, —CH₂—R^{2A}, or —R^{2A}. In some embodiments, R² is —N(H)—R^{2A}, —N(H)C(O)—R^{2A}, —CH₂N(H)—R^{2A}, or —R^{2A}. In some embodiments, R² is —N(H)—R^{2A}, —N(H)C(O)—R^{2A}, or —R^{2A}. In some embodiments, R² is —N(H)C(O)—R^{2A} or —R^{2A}.

In some embodiments, R² (i.e., -L²-R^{2A} taken together) is

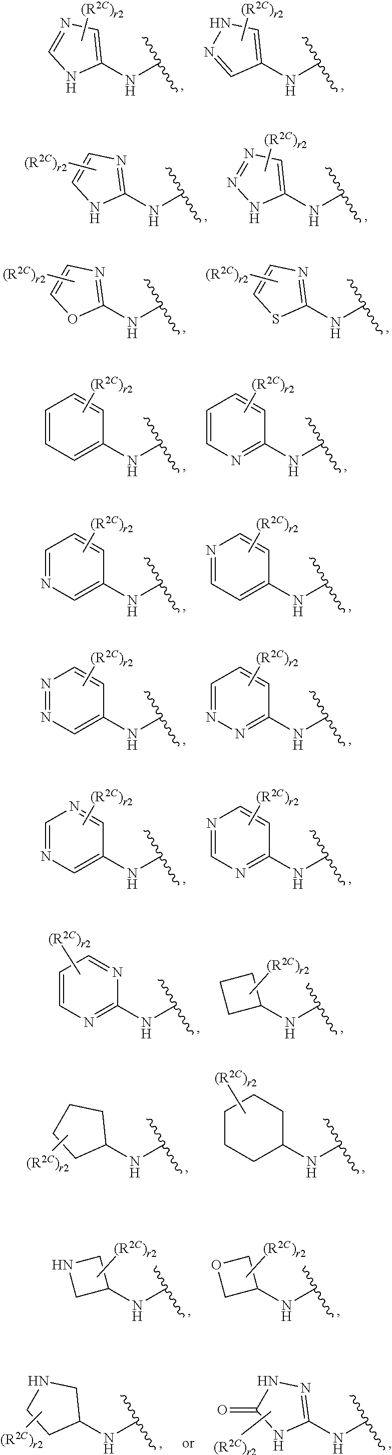

wherein R^{2C} and r² are as defined in the embodiments and classes and subclasses herein.

In some embodiments, R² (i.e., -L²-R^{2A} taken together) is

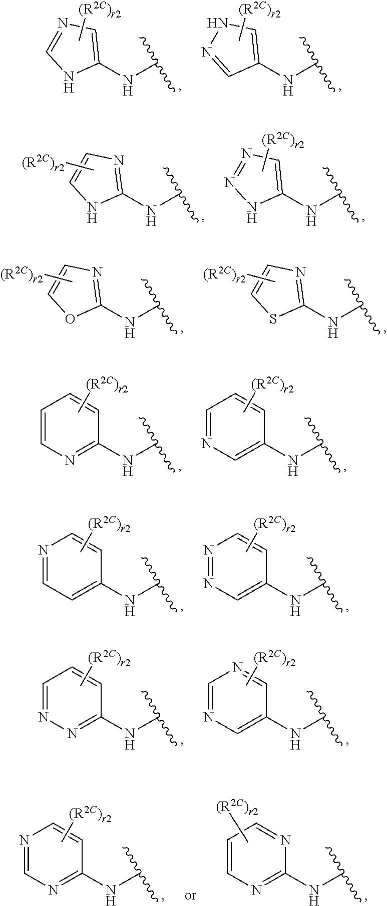

wherein R^{2C} and r² are as defined in the embodiments and classes and subclasses herein.

In some embodiments, R² (i.e., -L²-R^{2A} taken together) is

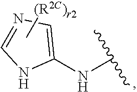

wherein R^{2C} and r² are as defined in the embodiments and classes and subclasses herein. In some embodiments, R² (i.e., -L²-R^{2A} taken together) is

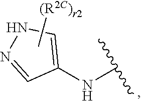

wherein R^{2C} and r² are as defined in the embodiments and classes and subclasses herein. In some embodiments, R² (i.e., -L²-R^{2A} taken together) is

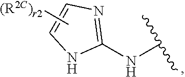

wherein R^{2C} and r² are as defined in the embodiments and classes and subclasses herein. In some embodiments, R² (i.e., -L²-R^{2A} taken together) is

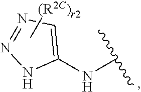

wherein R^{2C} and r² are as defined in the embodiments and classes and subclasses herein. In some embodiments, R² (i.e., -L²-R^{2A} taken together) is

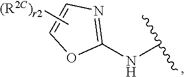

wherein R^{2C} and r² are as defined in the embodiments and classes and subclasses herein. In some embodiments, R² (i.e., -L²-R^{2A} taken together) is

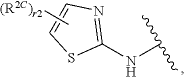

wherein R^{2C} and r² are as defined in the embodiments and classes and subclasses herein. In some embodiments, R² (i.e., -L²-R^{2A} taken together) is

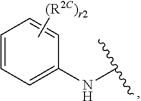

wherein R^{2C} and r² are as defined in the embodiments and classes and subclasses herein. In some embodiments, R² (i.e., -L²-R^{2A} taken together) is

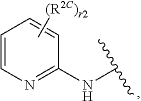

wherein R^{2C} and r² are as defined in the embodiments and classes and subclasses herein. In some embodiments, R² (i.e., -L²-R^{2A} taken together) is

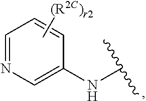

wherein R^{2C} and r² are as defined in the embodiments and classes and subclasses herein. In some embodiments, R² (i.e., -L²-R^{2A} taken together) is

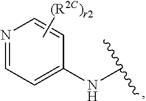

(wherein R^{2C} and r² are as defined in the embodiments and classes and subclasses herein. In some embodiments, R² (i.e., -L²-R^{2A} taken together) is

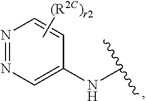

wherein R^{2C} and r² are as defined in the embodiments and classes and subclasses herein. In some embodiments, R² (i.e., -L²-R^{2A} taken together) is

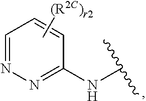

wherein R^{2C} and r² are as defined in the embodiments and classes and subclasses herein. In some embodiments, R² (i.e., -L²-R^{2A} taken together) is

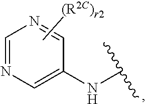

wherein R^{2C} and r² are as defined in the embodiments and classes and subclasses herein. In some embodiments, R² (i.e., -L²-R^{2A} taken together) is

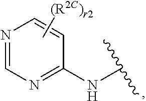

wherein R^{2C} and r² are as defined in the embodiments and classes and subclasses herein. In some embodiments, R² (i.e., -L²-R^{2A} taken together) is

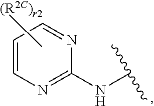

wherein R^{2C} and r² are as defined in the embodiments and classes and subclasses herein.

In some embodiments, R² (i.e., -L²-R^{2A} taken together) is

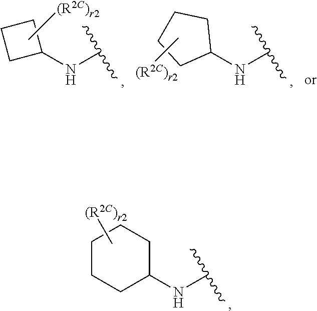

wherein R^{2C} and r² are as defined in the embodiments and classes and subclasses herein. In some embodiments, R² (i.e., -L²-R^{2A} taken together) is

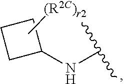

wherein R^{2C} and r² are as defined in the embodiments and classes and subclasses herein. In some embodiments, R² (i.e., -L²-R^{2A} taken together) is

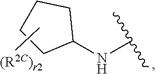

wherein R^{2C} and r² are as defined in the embodiments and classes and subclasses herein. In some embodiments, R² (i.e., -L²-R^{2A} taken together) is

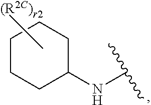

wherein R^{2C} and r² are as defined in the embodiments and classes and subclasses herein.

In some embodiments, R² (i.e., -L²-R^{2A} taken together) is

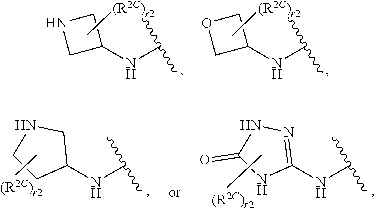

wherein R^{2C} and r² are as defined in the embodiments and classes and subclasses herein. In some embodiments, R² (i.e., -L²-R^{2A} taken together) is

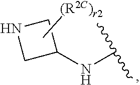

wherein R^{2C} and r² are as defined in the embodiments and classes and subclasses herein. In some embodiments, R² (i.e., -L²-R^{2A} taken together) is

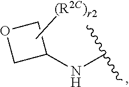

wherein R^{2C} and r² are as defined in the embodiments and classes and subclasses herein. In some embodiments, R² (i.e., -L²-R^{2A} taken together) is

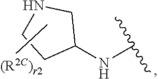

wherein R^{2C} and r² are as defined in the embodiments and classes and subclasses herein. In some embodiments, R² (i.e., -L²-R^{2A} taken together) is

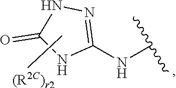

wherein R^{2C} and r² are as defined in the embodiments and classes and subclasses herein.

In some embodiments, R² (i.e., -L²-R^{2A} taken together) is

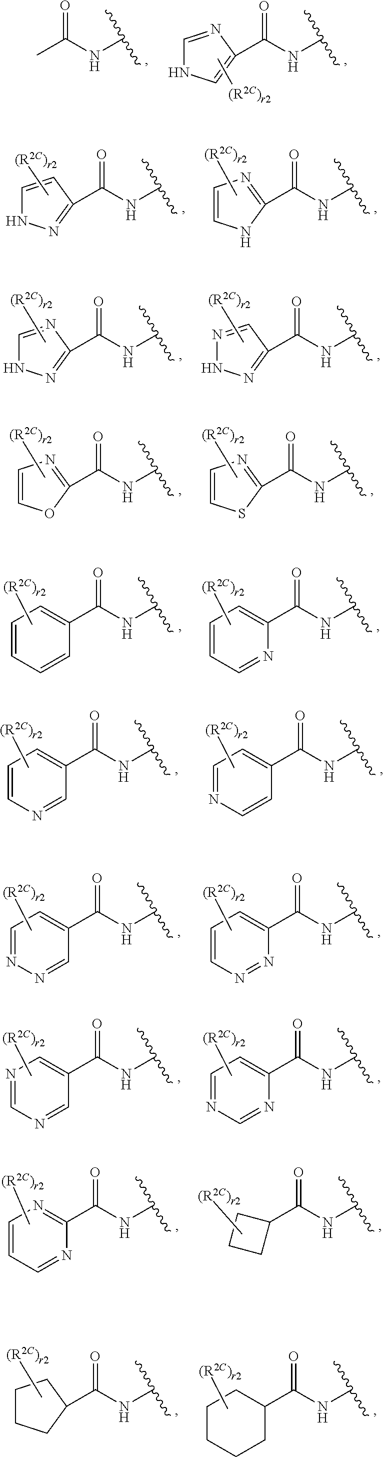

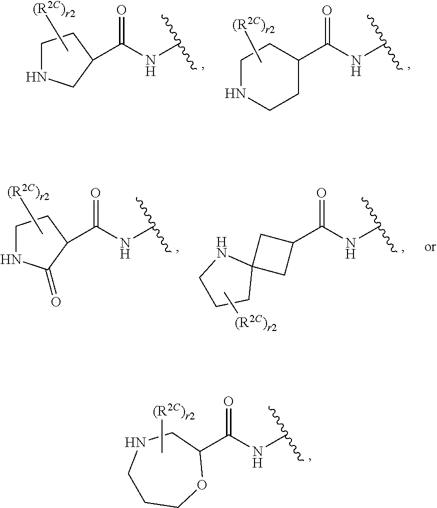

wherein R^{2C} and r² are as defined in the embodiments and classes and subclasses herein.

In some embodiments, R² (i.e., -L²-R^{2A} taken together) is

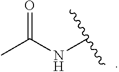 [SMILES: *NC(C)=O]

In some embodiments, R² (i.e., -L²-R^{2A} taken together) is

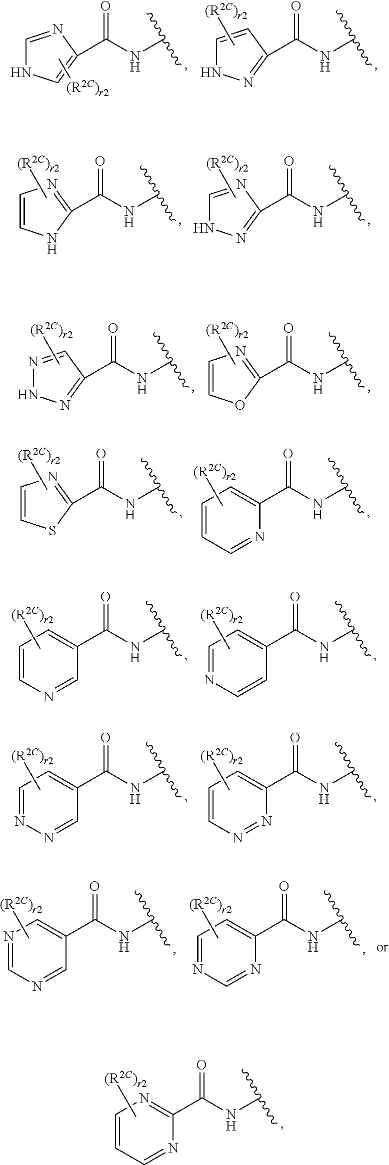

wherein R^{2C} and r² are as defined in the embodiments and classes and subclasses herein.

In some embodiments, R² (i.e., -L²-R^{2A} taken together) is

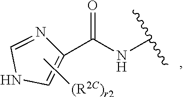

wherein R^{2C} and r² are as defined in the embodiments and classes and subclasses herein. In some embodiments, R² (i.e., -L²-R^{2A} taken together) is

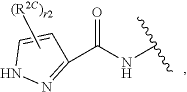

wherein R^{2C} and r² are as defined in the embodiments and classes and subclasses herein. In some embodiments, R² (i.e., -L²-R^{2A} taken together) is

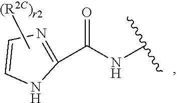

wherein R^{2C} and r² are as defined in the embodiments and classes and subclasses herein. In some embodiments, R² (i.e., -L²-R^{2A} taken together) is

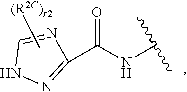

wherein R^{2C} and r² are as defined in the embodiments and classes and subclasses herein. In some embodiments, R² (i.e., -L²-R^{2A} taken together) is

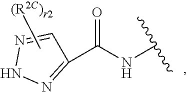

wherein R^{2C} and r² are as defined in the embodiments and classes and subclasses herein. In some embodiments, R² (i.e., -L²-R^{2A} taken together) is

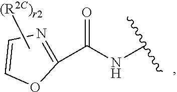

wherein R^{2C} and r² are as defined in the embodiments and classes and subclasses herein. In some embodiments, R² (i.e., -L²-R^{2A} taken together) is

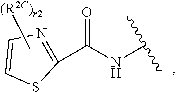

wherein R^{2C} and r² are as defined in the embodiments and classes and subclasses herein. In some embodiments, R² (i.e., -L²-R^{2A} taken together) is

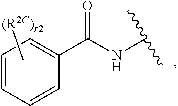

wherein R^{2C} and r² are as defined in the embodiments and classes and subclasses herein. In some embodiments, R² (i.e., -L²-R^{2A} taken together) is

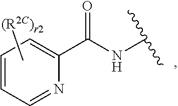

wherein R^{2C} and r² are as defined in the embodiments and classes and subclasses herein. In some embodiments, R² (i.e., -L²-R^{2A} taken together) is

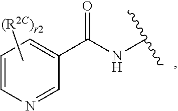

wherein R^{2C} and r² are as defined in the embodiments and classes and subclasses herein. In some embodiments, R² (i.e., -L²-R^{2A} taken together) is

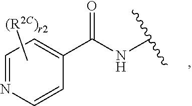

wherein R^{2C} and r² are as defined in the embodiments and classes and subclasses herein. In some embodiments, R² (i.e., -L²-R^{2A} taken together) is

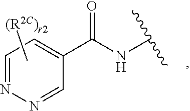

wherein R^{2C} and r² are as defined in the embodiments and classes and subclasses herein. In some embodiments, R² (i.e., -L²-R^{2A} taken together) is

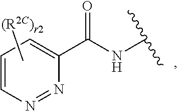

wherein R^{2C} and r² are as defined in the embodiments and classes and subclasses herein. In some embodiments, R² (i.e., -L²-R^{2A} taken together) is

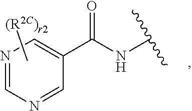

wherein R^{2C} and r² are as defined in the embodiments and classes and subclasses herein. In some embodiments, R² (i.e., -L²-R^{2A} taken together) is

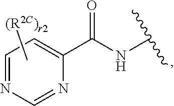

wherein R^{2C} and r² are as defined in the embodiments and classes and subclasses herein. In some embodiments, R² (i.e., -L²-R^{2A} taken together) is

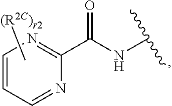

wherein R^{2C} and r² are as defined in the embodiments and classes and subclasses herein.

In some embodiments, R² (i.e., -L²-R^{2A} taken together) is

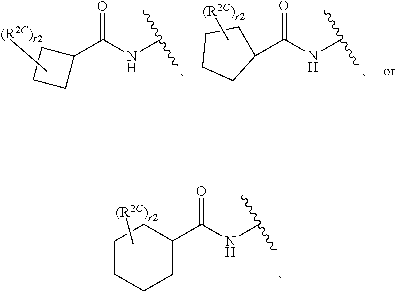

wherein R^{2C} and r² are as defined in the embodiments and classes and subclasses herein. In some embodiments, R² (i.e., -L²-R^{2A} taken together) is

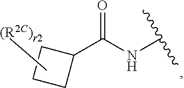

wherein R^{2C} and r² are as defined in the embodiments and classes and subclasses herein. In some embodiments, R² (i.e., -L²-R^{2A} taken together) is

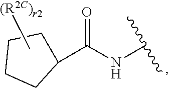

wherein R^{2C} and r² are as defined in the embodiments and classes and subclasses herein. In some embodiments, R² (i.e., -L²-R^{2A} taken together) is

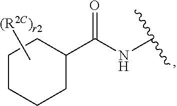

wherein R^{2C} and r² are as defined in the embodiments and classes and subclasses herein.

In some embodiments, R² (i.e., -L²-R^{2A} taken together) is

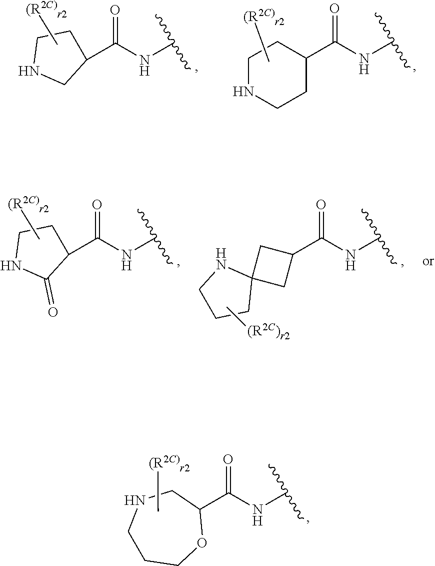

wherein R^{2C} and r² are as defined in the embodiments and classes and subclasses herein. In some embodiments, R² (i.e., -L²-R^{2A} taken together) is

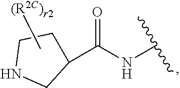

wherein R^{2C} and r² are as defined in the embodiments and classes and subclasses herein. In some embodiments, R² (i.e., -L²-R^{2A} taken together) is

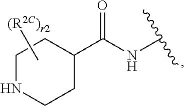

wherein R^{2C} and r² are as defined in the embodiments and classes and subclasses herein. In some embodiments, R² (i.e., -L²-R^{2A} taken together) is

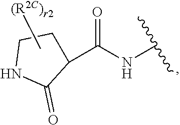

wherein R^{2C} and r² are as defined in the embodiments and classes and subclasses herein. In some embodiments, R² (i.e., -L²-R^{2A} taken together) is

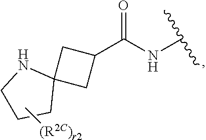

wherein R^{2C} and r² are as defined in the embodiments and classes and subclasses herein. In some embodiments, R² (i.e., -L²-R^{2A} taken together) is

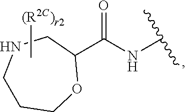

wherein R^{2C} and r² are as defined in the embodiments and classes and subclasses herein.

In some embodiments, R² (i.e., -L²-R^{2A} taken together) is

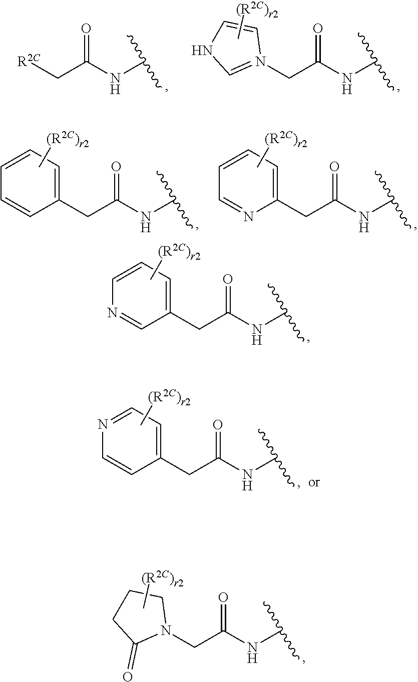

wherein R^{2C} and r² are as defined in the embodiments and classes and subclasses herein.

In some embodiments, R² (i.e., -L²-R^{2A} taken together) is

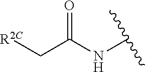

wherein R^{2C} is as defined in the embodiments and classes and subclasses herein. In some embodiments, R² (i.e., -L²-R^{2A} taken together) is

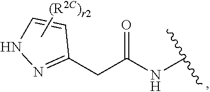

wherein R^{2C} and r² are as defined in the embodiments and classes and subclasses herein. In some embodiments, R² (i.e., -L²-R^{2A} taken together) is

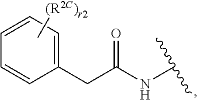

wherein R^{2C} and r² are as defined in the embodiments and classes and subclasses herein. In some embodiments, R² (i.e., -L²-R^{2A} taken together) is

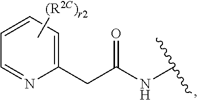

wherein R^{2C} and r² are as defined in the embodiments and classes and subclasses herein. In some embodiments, R² (i.e., -L²-R^{2A} taken together) is

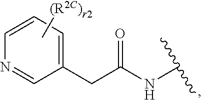

wherein R^{2C} and r² are as defined in the embodiments and classes and subclasses herein. In some embodiments, R² (i.e., -L²-R^{2A} taken together) is

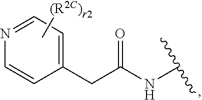

wherein R^{2C} and r² are as defined in the embodiments and classes and subclasses herein. In some embodiments, R² (i.e., -L²-R^{2A} taken together) is

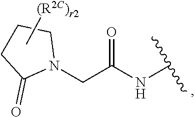

wherein R^{2C} and r² are as defined in the embodiments and classes and subclasses herein.

In some embodiments, R² (i.e., -L²-R^{2A} taken together) is

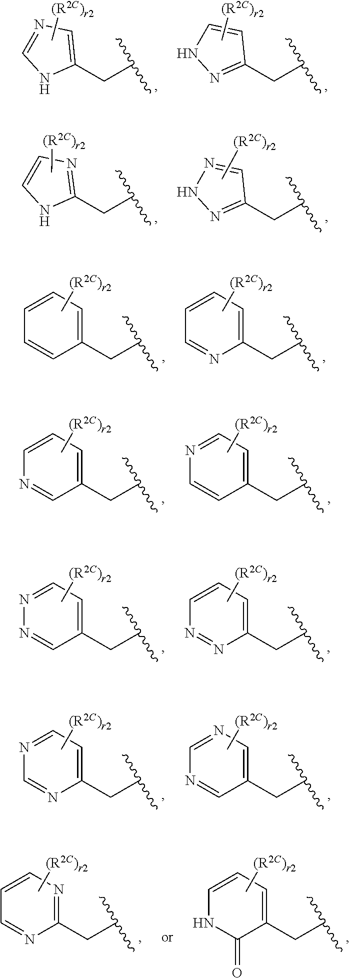

wherein R^{2A}, R^{2C}, and r² are as defined in the embodiments and classes and subclasses herein.

In some embodiments, R² (i.e., -L²-R^{2A} taken together) is

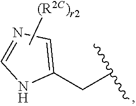

wherein R² and r² are as defined in embodiments and classes and subclasses herein. In some embodiments, R² (i.e., -L²-R^{2A} taken together) is

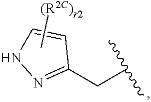

wherein R^{2C} and r² are as defined in embodiments and classes and subclasses herein. In some embodiments, R² (i.e., -L²-R^{2A}, taken together) is

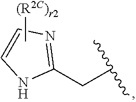

wherein R^{2C} and r² are as defined in embodiments and classes and subclasses herein. In some embodiments, R² (i.e., -L²-R^{2A} taken together) is

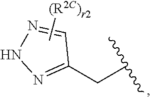

wherein R² and r² are as defined in embodiments and classes and subclasses herein. In some embodiments, R² (i.e., -L²-R^{2A} taken together) is

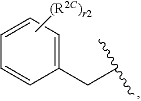

wherein R^{2C} and r² are as defined in embodiments and classes and subclasses herein. In some embodiments, R² (i.e., -L²-R^{2A} taken together) is

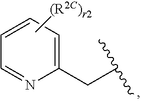

wherein R^{2C} and r² are as defined in embodiments and classes and subclasses herein. In some embodiments, R² (i.e., -L²-R^{2A} taken together) is

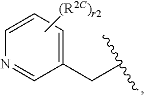

wherein R^{2C} and r² are as defined in embodiments and classes and subclasses herein. In some embodiments, R² (i.e., -L²-R^{2A} taken together) is

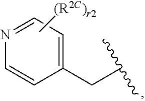

wherein R^{2C} and r² are as defined in embodiments and classes and subclasses herein. In some embodiments, R² (i.e., -L²-R^{2A} taken together) is

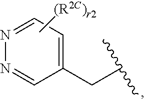

wherein R^{2C} and r² are as defined in embodiments and classes and subclasses herein. In some embodiments, R² (i.e., -L²-R^{2A} taken together) is

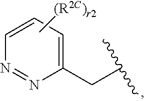

wherein R^{2C} and r² are as defined in embodiments and classes and subclasses herein. In some embodiments, R² (i.e., -L²-R^{2A} taken together) is

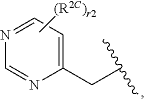

wherein R^{2C} and r² are as defined in embodiments and classes and subclasses herein. In some embodiments, R² (i.e., -L²-R^{2A} taken together) is

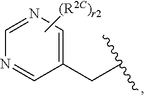

wherein R^{2C} and r² are as defined in embodiments and classes and subclasses herein. In some embodiments, R² (i.e., -L²-R^{2A} taken together) is

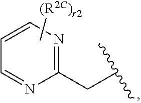

wherein R^{2C} and r² are as defined in embodiments and classes and subclasses herein. In some embodiments, R² (i.e., -L²-R^{2A} taken together) is

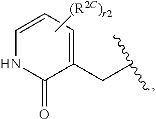

wherein R^{2C} and r² are as defined in embodiments and classes and subclasses herein.

In some embodiments, each R² (i.e., -L²-R^{2A} taken together) is independently halogen, —OH, —OCH₃, or C₁₋₃ aliphatic optionally substituted with 1-3 halogen. In some embodiments, each R² is independently fluorine, chlorine, —OCH₃, or —CH₃. In some embodiments, R² is —OH. In some embodiments, R² is —CH₃. In some embodiments, R² is —OCH₃. In some embodiments, R² is —CF₃. In some embodiments, R² is —CHF₂.

In some embodiments, two instances of R² are taken together with their intervening atoms to form a 3-7 membered saturated, partially unsaturated, or aromatic carbocyclic ring or a 3-7 membered saturated or partially unsaturated monocyclic heterocyclic ring having 1-3 heteroatoms independently selected from nitrogen, oxygen, and sulfur; wherein the ring is substituted with p³ instances of R^{22C}. In some embodiments, two instances of R² are taken together with their intervening atoms to form a 3-7 membered saturated, partially unsaturated, or aromatic carbocyclic ring; wherein the ring is substituted with p³ instances of R^{22C}. In some embodiments, two instances of R² are taken together with their intervening atoms to form a 6-membered aromatic carbocyclic ring; wherein the ring is substituted with p³ instances of R^{22C}.

In some embodiments, R² is selected from the groups depicted in the compounds in Table 1.

As defined generally above, each R^{T} is independently -L^{T}-R^{TA}; or two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC}; or one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}; or one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC}.

In some embodiments, each R^{T} is independently -L^{T}-R^{TA}. In some embodiments, each R^{T} is independently —R^{TA}. In some embodiments, each R^{T} is independently R^{A}. In some embodiments, each R^{T} is independently R^{B} substituted by r³ instances of R^{TC}.

In some embodiments, R^{T} (i.e., -L^{T}-R^{TA} taken together) is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a C₁₋₆ aliphatic chain; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a 3-7 membered saturated or partially unsaturated monocyclic carbocyclic ring or a 5-12 membered saturated or partially unsaturated bicyclic carbocyclic ring; each of which is substituted by r³ instances of R^{TC}. In some embodiments, R^{T} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted with r³ instances of R^{TC}.

In some embodiments, R^{T} is a 3-7 membered saturated monocyclic carbocyclic ring; a 5-12 membered saturated bicyclic carbocyclic ring; a 3-7 membered saturated monocyclic heterocyclic ring having 1-2 heteroatoms independently selected from nitrogen, oxygen, and sulfur; or a 7-12 membered saturated bicyclic heterocyclic ring having 1-4 heteroatoms independently selected from nitrogen, oxygen, and sulfur; each of which is substituted with r³ instances of R^{TC}. In some embodiments, R^{T} is a 3-7 membered saturated monocyclic carbocyclic ring; or a 5-12 membered saturated bicyclic carbocyclic ring; each of which is substituted with r³ instances of R^{TC}. In some embodiments, R^{T} is a 3-7 membered saturated monocyclic heterocyclic ring having 1-2 heteroatoms independently selected from nitrogen, oxygen, and sulfur; or a 7-12 membered saturated bicyclic heterocyclic ring having 1-4 heteroatoms independently selected from nitrogen, oxygen, and sulfur; each of which is substituted with r³ instances of R^{TC}.

In some embodiments, R^{T} (i.e., -L^{T}-R^{TA} taken together) is a C₁₋₆ aliphatic chain substituted with r³ instances of R^{TC}. In some embodiments, R^{T} (i.e., -LT-R^{TA} taken together) is phenyl substituted with r³ instances of R^{TC}. In some embodiments, R^{T} (i.e., -L^{T}-R^{TA} taken together) is naphthyl substituted with r³ instances of R^{TC}. In some embodiments, R^{T}(i.e., -L^{T}-R^{TA} taken together) is cubanyl substituted with r³ instances of R^{TC}. In some embodiments, R^{T} (i.e., -LT-R^{TA} taken together) is adamantyl substituted with r³ instances of R^{TC}. In some embodiments, R^{T} (i.e., -LT-R^{TA} taken together) is a 5-6 membered monocyclic heteroaryl ring having 1-4 heteroatoms independently selected from nitrogen, oxygen, and sulfur, wherein the ring is substituted with r³ instances of R^{TC}. In some embodiments, R^{T}(i.e., -LT-R^{TA} taken together) is an 8-10 membered bicyclic heteroaryl ring having 1-4 heteroatoms independently selected from nitrogen, oxygen, and sulfur, wherein the ring is substituted with r³ instances of R^{TC}. In some embodiments, R^{T} (i.e., -LT-R^{TA} taken together) is a 3-7 membered saturated or partially unsaturated monocyclic carbocyclic ring, wherein the ring is substituted with r³ instances of R^{TC}. In some embodiments, R^{T} (i.e., -L^{T}-R^{TA} taken together) is a 5-12 membered saturated or partially unsaturated bicyclic carbocyclic ring, wherein the ring is substituted with r³ instances of R^{TC}. In some embodiments, R^{T}(i.e., -LT-R^{TA} taken together) is a 3-7 membered saturated or partially unsaturated monocyclic heterocyclic ring having 1-2 heteroatoms independently selected from nitrogen, oxygen, and sulfur, wherein the ring is substituted with r³ instances of R^{TC}. In some embodiments, R^{T}(i.e., -L^{T}-R^{TA} taken together) is a 7-12 membered saturated or partially unsaturated bicyclic heterocyclic ring having 1-4 heteroatoms independently selected from nitrogen, oxygen, and sulfur, wherein the ring is substituted with r³ instances of R^{TC}.

In some embodiments R^{T} is

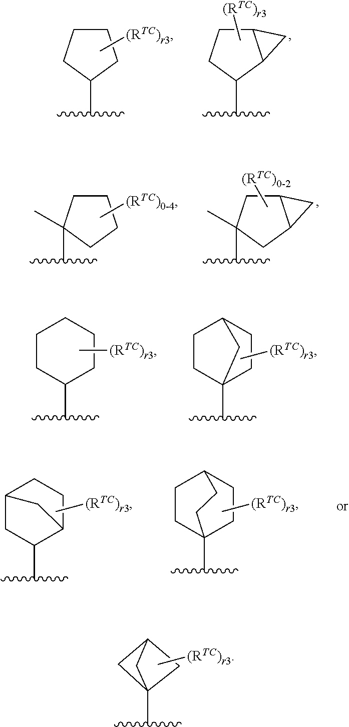

In some embodiments, R^{T} is

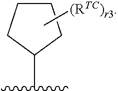

In some embodiments, R^{T} is

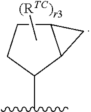

In some embodiments, R^{T} is

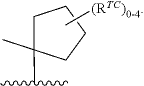

In some embodiments, R^{T} is

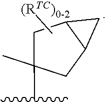

In some embodiments, R^{T} is

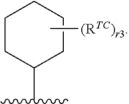

In some embodiments, R^{T} is

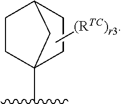

In some embodiments, R^{T} is

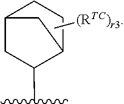

In some embodiments, R^{T} is

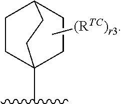

In some embodiments, R^{T} is

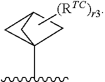

In some embodiments, R^{T} is CF3. In some embodiments, R^{T} is C₁₋₆ alkyl substituted by r³ instances of R^{TC}. In some embodiments, R^{T} is C₃₋₈ cycloalkyl substituted by r³ instances of R^{TC}.

In some embodiments, two instances of R^{T} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p¹ instances of R^{TTC}.

In some embodiments, one instance of R^{T} and one instance of R¹ are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁴ instances of R^{T1C}.

In some embodiments, one instance of R^{T} and one instance of R^{L} are taken together with their intervening atoms to form a 3-7 membered saturated or partially unsaturated carbocyclic ring or a 3-7 membered saturated or partially unsaturated monocyclic heterocyclic ring having 1-3 heteroatoms independently selected from nitrogen, oxygen, and sulfur; wherein the ring is substituted with p⁵ instances of R^{TLC}.

In some embodiments, R^{T} is selected from the groups depicted in the compounds in Table 1.

As defined generally above, L¹ is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a covalent bond. In some embodiments, L¹ is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₄ bivalent saturated or unsaturated, straight or branched hydrocarbon chain.

In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L¹ is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L¹ is a C₁₋₂ bivalent saturated or unsaturated hydrocarbon chain.

In some embodiments, L¹ is selected from the groups depicted in the compounds in Table 1.

As defined generally above, L² is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a covalent bond. In some embodiments, L² is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₄ bivalent saturated or unsaturated, straight or branched hydrocarbon chain.

In some embodiments, L² is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L² is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L² is a C₁₋₂ bivalent saturated or unsaturated hydrocarbon chain.

In some embodiments, L² is —N(R)—, —N(R)C(O)—, —CH(R^{L})N(R)—, —N(R)C(O)CH(R^{L})—, —CH(R^{L})O—, —CH(R^{L})—, or a covalent bond. In some embodiments, R² is —N(R)—, —N(R)C(O)—, —CH(R^{L})N(R)—, or a covalent bond. In some embodiments, R² is —N(R)—, —N(R)C(O)—, or a covalent bond. In some embodiments, R² is —N(R)C(O)— or a covalent bond.

In some embodiments, R² is —N(H)—, —N(H)C(O)—, —CH₂N(H)—, —N(H)C(O)CH₂—, —CH₂O—, —CH₂—, or a covalent bond. In some embodiments, R² is —N(H)—, —N(H)C(O)—, —CH₂N(H)—, or a covalent bond. In some embodiments, R² is —N(H)—, —N(H)C(O)—, or a covalent bond. In some embodiments, R² is —N(H)C(O)— or a covalent bond.

In some embodiments, L² is —N(R)C(O)— or —N(R)C(O)N(R)—. In some embodiments, L² is —N(H)C(O)— or —N(H)C(O)N(H)—. In some embodiments, L² is —N(R)C(O)—. In some embodiments, L² is —N(H)C(O)—. In some embodiments, L² is —N(R)C(O)N(R)—. In some embodiments, L² is —N(H)C(O)N(H)—. In some embodiments, L² is —N(R)—. In some embodiments, L² is —N(H)—. In some embodiments, L² is a covalent bond. In some embodiments, L² is —CH(R^{L})N(R)—. In some embodiments, L² is —N(R)C(O)CH(R^{L})—. In some embodiments, L² is —CH(R^{L})O—. In some embodiments, L² is —CH(R^{L})—.

In some embodiments, L² is selected from the groups depicted in the compounds in Table 1.

As defined generally above, L^{Q} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a covalent bond. In some embodiments, L^{Q} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₄ bivalent saturated or unsaturated, straight or branched hydrocarbon chain.

In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{Q} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{Q} is a C₁₋₂ bivalent saturated or unsaturated hydrocarbon chain.

In some embodiments, L^{Q} is —C(O)N(R)—, —C(O)N(R)CH₂—, —N(R)—, —CH₂C(O)N(R)—, —N(R)C(O)N(R)—, or a covalent bond. In some embodiments, L^{Q} is —C(O)N(H)—, —C(O)N(H)CH₂—, —N(H)—, —CH₂C(O)N(H)—, —N(H)C(O)N(H)—, or a covalent bond. In some embodiments, L^{Q} is —C(O)N(H)—, —C(O)N(H)CH₂—, or a covalent bond. In some embodiments, L^{Q} is —C(O)N(H)— or —C(O)N(H)CH₂—. In some embodiments, L^{Q} is —C(O)N(H)—. In some embodiments, L^{Q} is —C(O)N(H)CH₂—. In some embodiments, L^{Q} is —N(H)—. In some embodiments, L^{Q} is —CH₂C(O)N(H)—. In some embodiments, L^{Q} is —N(H)C(O)N(H)—. In some embodiments, L^{Q} is a covalent bond.

In some embodiments, L^{Q} is selected from the groups depicted in the compounds in Table 1.

As defined generally above, L^{T} is a covalent bond, or a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{T} is a covalent bond. In some embodiments, L^{T} is a C₁₋₄ bivalent saturated or unsaturated, straight or branched hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{X} is a C₁₋₄ bivalent saturated or unsaturated, straight, or branched hydrocarbon chain.

In some embodiments, L^{T} is a C₁₋₂ bivalent saturated or unsaturated hydrocarbon chain wherein one or two methylene units of the chain are optionally and independently replaced by —CH(R^{L})—, —C(R^{L})₂—, C₃₋₆ cycloalkylene, C₃₋₆ heterocycloalkylene, —N(R)—, —N(R)C(O)—, —C(O)N(R)—, —N(R)S(O)₂—, —S(O)₂N(R)—, —O—, —C(O)—, —OC(O)—, —C(O)O—, —S—, —S(O)—, or —S(O)₂—. In some embodiments, L^{T} is a C₁₋₂ bivalent saturated or unsaturated hydrocarbon chain wherein one or two methylene units of the chain are optionally and independently replaced by —CH(R^{L})—, —C(R^{L})₂—, —N(R)—, —N(R)C(O)—, —C(O)N(R)—, —N(R)S(O)₂—, —S(O)₂N(R)—, or —O—. In some embodiments, L^{T} is a C₁₋₂ bivalent saturated or unsaturated hydrocarbon chain.

In some embodiments, L^{T} is selected from the groups depicted in the compounds in Table 1.

As defined generally above, each R^{1A} is independently R^{A} or R^{B} substituted by r¹ instances of R^{1C}. In some embodiments, each R^{1A} is independently R^{A}. In some embodiments, each R^{1A} is independently R^{B} substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein RA is substituted by r¹ instances of R^{1C}.

In some embodiments, R^{1A} is phenyl substituted by r¹ instances of R^{1C}. In some embodiments, R^{1A} is an 8-10 membered bicyclic heteroaryl ring having 1-4 heteroatoms independently selected from nitrogen, oxygen, and sulfur, wherein RA is substituted by r¹ instances of R^{1C}. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; or a 3-7 membered saturated or partially unsaturated monocyclic carbocyclic ring; wherein R^{1A} is substituted by r¹ instances of R^{1C}.

In some embodiments, R^{1A} is phenyl substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{1A} is phenyl or an 8-10 membered bicyclic heteroaryl ring having 1-4 heteroatoms independently selected from nitrogen, oxygen, and sulfur, wherein R^{1A} is substituted by r¹ instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{1A} is phenyl substituted by 1-3 instances of R^{1C}. In some embodiments, R^{1A} is phenyl substituted by 2 instances of R^{1C}. In some embodiments, R^{1A} is phenyl substituted by 1 instance of R^{1C}.

In some embodiments, R^{1A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{1A} is phenyl substituted by 1-3 instances of a group independently selected from halogen and C₁₋₃ aliphatic optionally substituted with 1-3 halogen. In some embodiments, R^{1A} is phenyl substituted by 1-3 instances of a group independently selected from fluorine, chlorine, —CH₃, —CHF₂, and —CF₃.

In some embodiments, R^{1A} is phenyl substituted by 2 instances of a group independently selected from halogen, —CN, —O-(optionally substituted C₁₋₆ aliphatic), and an optionally substituted C₁₋₆ aliphatic. In some embodiments, R^{1A} is phenyl substituted by 2 instances of a group independently selected from halogen and C₁₋₃ aliphatic optionally substituted with 1-3 halogen. In some embodiments, R^{1A} is phenyl substituted by 2 instances of a group independently selected from fluorine, chlorine, —CH₃, —CHF₂, and —CF₃.

In some embodiments, R^{1A} is phenyl substituted by one group selected from halogen, —CN, —O-(optionally substituted C₁₋₆ aliphatic), and an optionally substituted C₁₋₆ aliphatic. In some embodiments, R^{1A} is phenyl substituted by one halogen or C₁₋₃ aliphatic group optionally substituted with 1-3 halogen. In some embodiments, R^{1A} is phenyl substituted by one fluorine, chlorine, —CH₃, —CHF₂, or —CF₃.

In some embodiments, each R^{1A} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, each R^{1A} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, each RA is independently halogen. In some embodiments, R^{1A} is —CN. In some embodiments, R^{1A} is —NO₂. In some embodiments, each R^{1A} is independently —OR. In some embodiments, each R^{1A} is independently —SR. In some embodiments, each R^{1A} is independently —NR₂. In some embodiments, each R^{1A} is independently —S(O)₂R. In some embodiments, each R^{1A} is independently —S(O)₂NR₂. In some embodiments, R^{1A} is —S(O)₂F. In some embodiments, each R^{1A} is independently —S(O)R. In some embodiments, each R^{1A} is independently —S(O)NR₂. In some embodiments, each R^{1A} is independently —S(O)(NR)R. In some embodiments, each R^{1A} is independently —C(O)R. In some embodiments, each R^{1A} is independently —C(O)OR. In some embodiments, each R^{1A} is independently —C(O)NR₂. In some embodiments, each R^{1A} is independently —C(O)N(R)OR. In some embodiments, each R^{1A} is independently —OC(O)R. In some embodiments, each R^{1A} is independently —OC(O)NR₂. In some embodiments, each R^{1A} is independently —N(R)C(O)OR. In some embodiments, each R^{1A} is independently —N(R)C(O)R. In some embodiments, each R^{1A} is independently —N(R)C(O)NR₂. In some embodiments, each R^{1A} is independently —N(R)C(NR)NR₂. In some embodiments, each R^{1A} is independently —N(R)S(O)₂NR₂. In some embodiments, each R^{1A} is independently —N(R)S(O)₂R. In some embodiments, each R^{1A} is independently —P(O)R₂. In some embodiments, each R^{1A} is independently —P(O)(R)OR. In some embodiments, each R^{1A} is independently —B(OR)₂. In some embodiments, R^{1A} is deuterium.

In some embodiments, R^{1A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{1A} is halogen, —CN, or —NO₂. In some embodiments, R^{1A} is —OR, —SR, or —NR₂. In some embodiments, R^{1A} is —S(O)₂R, —S(O)₂NR₂, —S(O)₂F, —S(O)R, —S(O)NR₂, or —S(O)(NR)R. In some embodiments, R^{1A} is —C(O)R, —C(O)OR, —C(O)NR₂, or —C(O)N(R)OR. In some embodiments, R^{1A} is —OC(O)R or —OC(O)NR₂. In some embodiments, R^{1A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{1A} is —P(O)R₂ or —P(O)(R)OR.

In some embodiments, R^{1A} is —OR, —OC(O)R, or —OC(O)NR₂. In some embodiments, R^{1A} is —SR, —S(O)₂R, —S(O)₂NR₂, —S(O)₂F, —S(O)R, —S(O)NR₂, or —S(O)(NR)R. In some embodiments, R^{1A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{1A} is —S(O)₂R, —S(O)₂NR₂, or —S(O)₂F. In some embodiments, R^{1A} is —S(O)R, —S(O)NR₂, or —S(O)(NR)R. In some embodiments, R^{1A} is —SR, —S(O)₂R, or —S(O)R. In some embodiments, R^{1A} is —S(O)₂NR₂, —S(O)NR₂, or —S(O)(NR)R. In some embodiments, R^{1A} is —S(O)₂NR₂ or —S(O)NR₂. In some embodiments, R^{1A} is —SR, —S(O)₂R, —S(O)₂NR₂, or —S(O)R.

In some embodiments, R^{1A} is —N(R)C(O)OR, —N(R)C(O)R, or —N(R)C(O)NR₂. In some embodiments, R^{1A} is —N(R)S(O)₂NR₂ or —N(R)S(O)₂R. In some embodiments, R^{1A} is —N(R)C(O)OR or —N(R)C(O)R. In some embodiments, R^{1A} is —N(R)C(O)NR₂ or —N(R)S(O)₂NR₂. In some embodiments, R^{1A} is —N(R)C(O)OR, —N(R)C(O)R, or —N(R)S(O)₂R.

In some embodiments, R^{1A} is —NR₂, —N(R)C(O)OR, —N(R)C(O)R, or —N(R)C(O)NR₂. In some embodiments, R^{1A} is —NR₂, —N(R)C(O)OR, or —N(R)C(O)R. In some embodiments, R^{1A} is —NR₂, —N(R)C(O)OR, —N(R)C(O)R, or —N(R)S(O)₂R.

In some embodiments, R^{1A} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain substituted by r¹ instances of R^{1C}. In some embodiments, R^{1A} is phenyl substituted by r¹ instances of R^{1C}. In some embodiments, R^{1A} is naphthyl substituted by r¹ instances of R^{1C}. In some embodiments, R^{1A} is a 5-6 membered monocyclic heteroaryl ring having 1-4 heteroatoms independently selected from nitrogen, oxygen, and sulfur; wherein said ring is substituted by r¹ instances of R^{1C}. In some embodiments, R^{1A} is an 8-10 membered bicyclic heteroaryl ring having 1-4 heteroatoms independently selected from nitrogen, oxygen, and sulfur; wherein said ring is substituted by r¹ instances of R^{1C}. In some embodiments, R^{1A} is a 3-7 membered saturated or partially unsaturated monocyclic carbocyclic ring substituted by r¹ instances of R^{1C}. In some embodiments, R^{1A} is a 5-12 membered saturated or partially unsaturated bicyclic carbocyclic ring substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; wherein said ring is substituted by r¹ instances of R^{1C}. In some embodiments, R^{1A} is a 7-12 membered saturated or partially unsaturated bicyclic heterocyclic ring having 1-4 heteroatoms independently selected from nitrogen, oxygen, and sulfur; wherein said ring is substituted by r¹ instances of R^{1C}.

In some embodiments, R^{1A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or naphthyl; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 5-12 membered saturated or partially unsaturated bicyclic carbocyclic ring; each of which is substituted by r¹ instances of R^{1C}. In some embodiments, R^{1A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 5-6 membered monocyclic heteroaryl ring having 1-4 heteroatoms independently selected from nitrogen, oxygen, and sulfur; each of which is substituted by r¹ instances of R^{1C}. In some embodiments, R^{1A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is naphthyl or an 8-10 membered bicyclic heteroaryl ring having 1-4 heteroatoms independently selected from nitrogen, oxygen, and sulfur; each of which is substituted by r¹ instances of R^{1C}. In some embodiments, R^{1A} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is phenyl or a 3-7 membered saturated or partially unsaturated monocyclic carbocyclic ring; each of which is substituted by r¹ instances of R^{1C}. In some embodiments, R^{1A} is naphthyl or a 5-12 membered saturated or partially unsaturated bicyclic carbocyclic ring; each of which is substituted by r¹ instances of R^{1C}. In some embodiments, R^{1A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}.

In some embodiments, R^{1A} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r¹ instances of R^{1C}. In some embodiments, R^{1A} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r¹ instances of R^{1C}. In some embodiments, R^{1A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r¹ instances of R^{1C}.

In some embodiments, R^{1A} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r¹ instances of R^{1C}. In some embodiments, R^{1A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r¹ instances of R^{1C}. In some embodiments, R^{1A} is a C₁₋₆ aliphatic chain, phenyl, or a 3-7 membered saturated or partially unsaturated monocyclic carbocyclic ring; each of which is substituted by r¹ instances of R^{1C}.

In some embodiments, each R^{1A} is independently halogen, —CN, —OR, or a C₁₋₆ aliphatic chain substituted with r¹ halogens. In some embodiments, each R^{1A} is independently halogen, —CN, —OR, or a C₁₋₆ aliphatic chain substituted with 0-5 halogens. In some embodiments, each R^{1A} is independently halogen, —CN, —O—(C₁₋₆ aliphatic chain substituted with 0-5 halogens), or a C₁₋₆ aliphatic chain substituted with 0-5 halogens. In some embodiments, each R^{1A} is independently halogen or a C₁₋₆ aliphatic chain substituted with 0-5 halogens. In some embodiments, each R^{1A} is independently halogen or a C₁₋₆ aliphatic chain substituted with 0-4 halogens. In some embodiments, each R^{1A} is independently halogen or a C₁₋₆ aliphatic chain substituted with 0-3 halogens. In some embodiments, each R^{1A} is independently halogen or a C₁₋₃ aliphatic chain substituted with 0-3 halogens. In some embodiments, each R^{1A} is independently halogen or a C₁₋₃ aliphatic chain substituted with 0-2 halogens.

In some embodiments, each R^{1A} is independently a halogen selected from Br, Cl, and F. In some embodiments, each R^{1A} is independently a halogen selected from Cl and F. In some embodiments, R^{1A} is Cl. In some embodiments, R^{1A} is F.

In some embodiments, at least one R^{1A} is halogen. In some embodiments, at least two R^{1A} are halogen. In some embodiments, at least three R^{1A} are halogen. In some embodiments one instance of R^{1A} is Cl. In some embodiments two instances of R^{1A} are Cl. In some embodiments, one instance of R^{1A} is F. In some embodiments, two instances of R^{1A} are F. In some embodiments, one instance of R^{1A} is Cl, and one instance of R^{1A} is F. In some embodiments, two instances of R^{1A} are Cl, and one instance of R^{1A} is F. In some embodiments, one instance of R^{1A} is Cl, and two instances of R^{1A} are F.

In some embodiments, R^{1A} is a C₁₋₆ aliphatic chain substituted with 0-5 halogens. In some embodiments, R^{1A} is a C₁₋₆ aliphatic chain substituted with 0-4 halogens. In some embodiments, R^{1A} is a C₁₋₆ aliphatic chain substituted with 0-3 halogens. In some embodiments, R^{1A} is a C₁₋₃ aliphatic chain substituted with 0-3 halogens. In some embodiments, R^{1A} is a C₁₋₃ aliphatic chain substituted with 0-2 halogens.

In some embodiments, at least one R^{1A} is C₁₋₃ aliphatic optionally substituted with 1-3 halogen. In some embodiments, at least one R^{1A} is —O—C₁₋₃ aliphatic optionally substituted with 1-3 halogen.

In some embodiments, each R^{1A} is independently halogen, —OH, —OCH₃, or C₁₋₃ aliphatic optionally substituted with 1-3 halogen. In some embodiments, each R^{1A} is independently fluorine, chlorine, —OCH₃, or —CH₃. In some embodiments, R^{1A} is —OH. In some embodiments, R^{1A} is —CH₃. In some embodiments, R^{1A} is —OCH₃. In some embodiments, R^{1A} is —CF₃. In some embodiments, R^{1A} is —CHF₂.

In some embodiments, R^{1A} is selected from the groups depicted in the compounds in Table 1.

As defined generally above, each R^{2A} is independently R^{A} or R^{B} substituted by r² instances of R^{2C}. In some embodiments, each R^{2A} is R^{A}. In some embodiments, each R^{2A} is R^{B} substituted by r² instances of R^{2C}.

In some embodiments, R^{2A} is phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of R^{2C}. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of R^{2C}.

In some embodiments, R^{2A} is phenyl; naphthyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl; an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic. In some embodiments, R^{2A} is phenyl or an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by r² instances of R^{2C}. In some embodiments, R^{2A} is phenyl substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is phenyl substituted by 1-3 instances of a group independently selected from halogen, —CN, —O-(optionally substituted C₁₋₆ aliphatic), and an optionally substituted C₁₋₆ aliphatic. In some embodiments, R^{2A} is phenyl substituted by 1-3 instances of a group independently selected from halogen and C₁₋₃ aliphatic optionally substituted with 1-3 halogen. In some embodiments, R^{2A} is phenyl substituted by 1-3 instances of a group independently selected from fluorine, chlorine, —CH₃, —CHF₂, and —CF₃.

In some embodiments, R^{2A} is phenyl substituted by 2 instances of a group independently selected from halogen, —CN, —O-(optionally substituted C₁₋₆ aliphatic), and an optionally substituted C₁₋₆ aliphatic. In some embodiments, R^{2A} is phenyl substituted by 2 instances of a group independently selected from halogen and C₁₋₃ aliphatic optionally substituted with 1-3 halogen. In some embodiments, R^{2A} is phenyl substituted by 2 instances of a group independently selected from fluorine, chlorine, —CH₃, —CHF₂, and —CF₃.

In some embodiments, R^{2A} is an 8-10 membered bicyclic heteroaryl ring having 1-4 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-4 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by r² instances of R^{2C}. In some embodiments, R^{2A} is an 8-10 membered bicyclic heteroaryl ring having 1-2 heteroatoms independently selected from nitrogen, oxygen, and sulfur; wherein R^{2A} is substituted by r² instances of a group independently selected from oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂, and optionally substituted C₁₋₆ aliphatic.

In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen, —CN, —O-(optionally substituted C₁₋₆ aliphatic), and an optionally substituted C₁₋₆ aliphatic. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from halogen and C₁₋₃ aliphatic optionally substituted with 1-3 halogen. In some embodiments, R^{2A} is an 8-10 membered bicyclic heteroaryl ring having 1-2 heteroatoms independently selected from nitrogen, oxygen, and sulfur; wherein R^{2A} is substituted by 0-2 instances of a group independently selected from fluorine, chlorine, —CH₃, —CHF₂, and —CF₃.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{2A} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is oxo. In some embodiments, R^{2A} is halogen. In some embodiments, R^{2A} is —CN. In some embodiments, R^{2A} is —NO₂. In some embodiments, R^{2A} is —OR. In some embodiments, R^{2A} is —SR. In some embodiments, R^{2A} is —NR₂. In some embodiments, R^{2A} is —S(O)₂R. In some embodiments, R^{2A} is —S(O)₂NR₂. In some embodiments, R^{2A} is —S(O)₂F. In some embodiments, R^{2A} is —S(O)R. In some embodiments, R^{2A} is —S(O)NR₂. In some embodiments, R^{2A} is —S(O)(NR)R. In some embodiments, R^{2A} is —C(O)R. In some embodiments, R^{2A} is —C(O)OR. In some embodiments, R^{2A} is —C(O)NR₂. In some embodiments, R^{2A} is —C(O)N(R)OR. In some embodiments, R^{2A} is —OC(O)R. In some embodiments, R^{2A} is —OC(O)NR₂. In some embodiments, R^{2A} is —N(R)C(O)OR. In some embodiments, R^{2A} is —N(R)C(O)R. In some embodiments, R^{2A} is —N(R)C(O)NR₂. In some embodiments, R^{2A} is —N(R)C(NR)NR₂. In some embodiments, R^{2A} is —N(R)S(O)₂NR₂. In some embodiments, R^{2A} is —N(R)S(O)₂R. In some embodiments, R^{2A} is —P(O)R₂. In some embodiments, R^{2A} is —P(O)(R)OR. In some embodiments, R^{2A} is —B(OR)₂. In some embodiments, R^{2A} is deuterium.

In some embodiments, R^{2A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{2A} is halogen, —CN, or —NO₂. In some embodiments, R^{2A} is —OR, —SR, or —NR₂. In some embodiments, R^{2A} is —S(O)₂R, —S(O)₂NR₂, —S(O)₂F, —S(O)R, —S(O)NR₂, or —S(O)(NR)R. In some embodiments, R^{2A} is —C(O)R, —C(O)OR, —C(O)NR₂, or —C(O)N(R)OR. In some embodiments, R^{2A} is —OC(O)R or —OC(O)NR₂. In some embodiments, R^{2A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{2A} is —P(O)R₂ or —P(O)(R)OR.

In some embodiments, R^{2A} is —OR, —OC(O)R, or —OC(O)NR₂. In some embodiments, R^{2A} is —SR, —S(O)₂R, —S(O)₂NR₂, —S(O)₂F, —S(O)R, —S(O)NR₂, or —S(O)(NR)R. In some embodiments, R^{2A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{2A} is —S(O)₂R, —S(O)₂NR₂, or —S(O)₂F. In some embodiments, R^{2A} is —S(O)R, —S(O)NR₂, or —S(O)(NR)R. In some embodiments, R^{2A} is —SR, —S(O)₂R, or —S(O)R. In some embodiments, R^{2A} is —S(O)₂NR₂, —S(O)NR₂, or —S(O)(NR)R. In some embodiments, R^{2A} is —S(O)₂NR₂ or —S(O)NR₂. In some embodiments, R^{2A} is —SR, —S(O)₂R, —S(O)₂NR₂, or —S(O)R.

In some embodiments, R^{2A} is —N(R)C(O)OR, —N(R)C(O)R, or —N(R)C(O)NR₂. In some embodiments, R^{2A} is —N(R)S(O)₂NR₂ or —N(R)S(O)₂R. In some embodiments, R^{2A} is —N(R)C(O)OR or —N(R)C(O)R. In some embodiments, R^{2A} is —N(R)C(O)NR₂ or —N(R)S(O)₂NR₂. In some embodiments, R^{2A} is —N(R)C(O)OR, —N(R)C(O)R, or —N(R)S(O)₂R.

In some embodiments, R^{2A} is —NR₂, —N(R)C(O)OR, —N(R)C(O)R, or —N(R)C(O)NR₂. In some embodiments, R^{2A} is —NR₂, —N(R)C(O)OR, or —N(R)C(O)R. In some embodiments, R^{2A} is —NR₂, —N(R)C(O)OR, —N(R)C(O)R, or —N(R)S(O)₂R.

In some embodiments, R^{2A} is a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain substituted by r² instances of R^{2C}. In some embodiments, R^{2A} is phenyl substituted by r² instances of R^{2C}. In some embodiments, R^{2A} is naphthyl substituted by r² instances of R^{2C}. In some embodiments, R^{2A} is cubanyl substituted by r² instances of R^{2C}. In some embodiments, R^{2A} is adamantyl substituted by r² instances of R^{2C}. In some embodiments, R^{2A} is a 5-6 membered monocyclic heteroaryl ring having 1-4 heteroatoms independently selected from nitrogen, oxygen, and sulfur; wherein said ring is substituted by r² instances of R^{2C}. In some embodiments, R^{2A} is an 8-10 membered bicyclic heteroaryl ring having 1-4 heteroatoms independently selected from nitrogen, oxygen, and sulfur; wherein said ring is substituted by r² instances of R^{2C}. In some embodiments, R^{2A} is a 3-7 membered saturated or partially unsaturated monocyclic carbocyclic ring substituted by r² instances of R^{2C}. In some embodiments, R^{2A} is a 5-12 membered saturated or partially unsaturated bicyclic carbocyclic ring substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; wherein said ring is substituted by r² instances of R^{2C}. In some embodiments, R^{2A} is a 7-12 membered saturated or partially unsaturated bicyclic heterocyclic ring having 1-4 heteroatoms independently selected from nitrogen, oxygen, and sulfur; wherein said ring is substituted by r² instances of R^{2C}.

In some embodiments, R^{2A} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl; cubanyl; adamantyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or naphthyl; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 5-12 membered saturated or partially unsaturated bicyclic carbocyclic ring; each of which is substituted by r² instances of R^{2C}. In some embodiments, R^{2A} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 5-6 membered monocyclic heteroaryl ring having 1-4 heteroatoms independently selected from nitrogen, oxygen, and sulfur; each of which is substituted by r² instances of R^{2C}. In some embodiments, R^{2A} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is naphthyl or an 8-10 membered bicyclic heteroaryl ring having 1-4 heteroatoms independently selected from nitrogen, oxygen, and sulfur; each of which is substituted by r² instances of R^{2C}. In some embodiments, R^{2A} is cubanyl; adamantyl; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is phenyl or a 3-7 membered saturated or partially unsaturated monocyclic carbocyclic ring; each of which is substituted by r² instances of R^{2C}. In some embodiments, R^{2A} is naphthyl; cubanyl; adamantyl; or a 5-12 membered saturated or partially unsaturated bicyclic carbocyclic ring; each of which is substituted by r² instances of R^{2C}. In some embodiments, R^{2A} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}.

In some embodiments, R^{2A} is a C₁₋₆ aliphatic chain; cubanyl; adamantyl; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r² instances of R^{2C}. In some embodiments, R^{2A} is a C₁₋₆ aliphatic chain; phenyl; naphthyl; cubanyl; adamantyl; a 3-7 membered saturated or partially unsaturated monocyclic carbocyclic ring; or a 5-12 membered saturated or partially unsaturated bicyclic carbocyclic ring; each of which is substituted by r² instances of R^{2C}. In some embodiments, R^{2A} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r² instances of R^{2C}.

In some embodiments, R^{2A} is a C₁₋₆ aliphatic chain, cubanyl, adamantyl, a 3-7 membered saturated or partially unsaturated monocyclic carbocyclic ring, or a 5-12 membered saturated or partially unsaturated bicyclic carbocyclic ring; each of which is substituted by r² instances of R^{2C}. In some embodiments, R^{2A} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r² instances of R^{2C}. In some embodiments, R^{2A} is a C₁₋₆ aliphatic chain, phenyl, or a 3-7 membered saturated or partially unsaturated monocyclic carbocyclic ring; each of which is substituted by r² instances of R^{2C}.

In some embodiments, R^{2A} is selected from the groups depicted in the compounds in Table 1.

As defined generally above, each R^{TA} is independently R^{A} or R^{B} substituted with r³ instances of R^{TC}. In some embodiments, each R^{T} is independently R^{A}. In some embodiments, each R^{T} is independently R^{B} substituted with r³ instances of R^{TC}.

In some embodiments, R^{TA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{TA} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{TA} is oxo. In some embodiments, R^{TA} is halogen. In some embodiments, RTA is —CN. In some embodiments, R^{TA} is —NO₂. In some embodiments, R^{TA} is —OR. In some embodiments, R^{TA} is —SR. In some embodiments, R^{TA} is —NR₂. In some embodiments, R^{TA} is —S(O)₂R. In some embodiments, R^{TA} is —S(O)₂NR₂. In some embodiments, R^{TA} is —S(O)₂F. In some embodiments, R^{TA} is —S(O)R. In some embodiments, R^{TA} is —S(O)NR₂. In some embodiments, R^{TA} is —S(O)(NR)R. In some embodiments, R^{TA} is —C(O)R. In some embodiments, R^{TA} is —C(O)OR. In some embodiments, R^{TA} is —C(O)NR₂. In some embodiments, R^{TA} is —C(O)N(R)OR. In some embodiments, R^{TA} is —OC(O)R. In some embodiments, R^{TA} is —OC(O)NR₂. In some embodiments, R^{TA} is —N(R)C(O)OR. In some embodiments, R^{TA} is —N(R)C(O)R. In some embodiments, R^{TA} is —N(R)C(O)NR₂. In some embodiments, R^{TA} is —N(R)C(NR)NR₂. In some embodiments, R^{TA} is —N(R)S(O)₂NR₂. In some embodiments, R^{TA} is —N(R)S(O)₂R. In some embodiments, R^{TA} is —P(O)R₂. In some embodiments, R^{TA} is —P(O)(R)OR. In some embodiments, R^{TA} is —B(OR)₂. In some embodiments, R^{TA} is deuterium.

In some embodiments, R^{TA} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{TA} is halogen, —CN, or —NO₂. In some embodiments, R^{TA} is —OR, —SR, or —NR₂. In some embodiments, R^{TA} is —S(O)₂R, —S(O)₂NR₂, —S(O)₂F, —S(O)R, —S(O)NR₂, or —S(O)(NR)R. In some embodiments, R^{TA} is —C(O)R, —C(O)OR, —C(O)NR₂, or —C(O)N(R)OR. In some embodiments, R^{TA} is —OC(O)R or —OC(O)NR₂. In some embodiments, R^{TA} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{TA} is —P(O)R₂ or —P(O)(R)OR.

In some embodiments, R^{TA} is —OR, —OC(O)R, or —OC(O)NR₂. In some embodiments, R^{TA} is —SR, —S(O)₂R, —S(O)₂NR₂, —S(O)₂F, —S(O)R, —S(O)NR₂, or —S(O)(NR)R. In some embodiments, R^{TA} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{TA} is —S(O)₂R, —S(O)₂NR₂, or —S(O)₂F. In some embodiments, R^{TA} is —S(O)R, —S(O)NR₂, or —S(O)(NR)R. In some embodiments, R^{TA} is —SR, —S(O)₂R, or —S(O)R. In some embodiments, R^{TA} is —S(O)₂NR₂, —S(O)NR₂, or —S(O)(NR)R. In some embodiments, R^{TA} is —S(O)₂NR₂ or —S(O)NR₂. In some embodiments, R^{TA} is —SR, —S(O)₂R, —S(O)₂NR₂, or —S(O)R.

In some embodiments, R^{TA} is —N(R)C(O)OR, —N(R)C(O)R, or —N(R)C(O)NR₂. In some embodiments, R^{TA} is —N(R)S(O)₂NR₂ or —N(R)S(O)₂R. In some embodiments, R^{TA} is —N(R)C(O)OR or —N(R)C(O)R. In some embodiments, R^{TA} is —N(R)C(O)NR₂ or —N(R)S(O)₂NR₂. In some embodiments, R^{TA} is —N(R)C(O)OR, —N(R)C(O)R, or —N(R)S(O)₂R.

In some embodiments, R^{TA} is —NR₂, —N(R)C(O)OR, —N(R)C(O)R, or —N(R)C(O)NR₂. In some embodiments, R^{TA} is —NR₂, —N(R)C(O)OR, or —N(R)C(O)R. In some embodiments, R^{TA} is —NR₂, —N(R)C(O)OR, —N(R)C(O)R, or —N(R)S(O)₂R.

In some embodiments, R^{TA} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is a C₁₋₆ aliphatic chain substituted by r³ instances of R^{TC}. In some embodiments, R^{TA} is phenyl substituted by r³ instances of R^{TC}. In some embodiments, R^{TA} is naphthyl substituted by r³ instances of R^{TC}. In some embodiments, R^{TA} is a 5-6 membered monocyclic heteroaryl ring having 1-4 heteroatoms independently selected from nitrogen, oxygen, and sulfur; wherein said ring is substituted by r³ instances of R^{TC} In some embodiments, R^{TA} is an 8-10 membered bicyclic heteroaryl ring having 1-4 heteroatoms independently selected from nitrogen, oxygen, and sulfur; wherein said ring is substituted by r³ instances of R^{TC}. In some embodiments, R^{TA} is a 3-7 membered saturated or partially unsaturated monocyclic carbocyclic ring substituted by r³ instances of R^{TC}. In some embodiments, R^{TA} is a 5-12 membered saturated or partially unsaturated bicyclic carbocyclic ring substituted by r³ instances of R^{TC}. In some embodiments, R^{TA} is a 3-7 membered saturated or partially unsaturated monocyclic heterocyclic ring having 1-2 heteroatoms independently selected from nitrogen, oxygen, and sulfur; wherein said ring is substituted by r³ instances of R^{TC}. In some embodiments, R^{TA} is a 7-12 membered saturated or partially unsaturated bicyclic heterocyclic ring having 1-4 heteroatoms independently selected from nitrogen, oxygen, and sulfur; wherein said ring is substituted by r³ instances of R^{TC}.

In some embodiments, R^{TA} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{TC} In some embodiments, R^{TA} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or naphthyl; each of which is substituted by r³ instances of R^{TC}. In some embodiments, R^{TA} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring or a 5-12 membered saturated or partially unsaturated bicyclic carbocyclic ring; each of which is substituted by r³ instances of R^{T}. In some embodiments, R^{TA} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or a 5-6 membered monocyclic heteroaryl ring having 1-4 heteroatoms independently selected from nitrogen, oxygen, and sulfur; each of which is substituted by r³ instances of R^{TC}. In some embodiments, R^{TA} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is naphthyl or an 8-10 membered bicyclic heteroaryl ring having 1-4 heteroatoms independently selected from nitrogen, oxygen, and sulfur; each of which is substituted by r³ instances of R^{TC}. In some embodiments, R^{TA} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is phenyl or a 3-7 membered saturated or partially unsaturated monocyclic carbocyclic ring; each of which is substituted by r³ instances of R^{TC}. In some embodiments, R^{TA} is naphthyl or a 5-12 membered saturated or partially unsaturated bicyclic carbocyclic ring; each of which is substituted by r³ instances of R^{TC}. In some embodiments, R^{TA} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r³ instances of R^{TC}. In some embodiments, R^{TA} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}.

In some embodiments, R^{TA} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r³ instances of R^{TC}. In some embodiments, R^{TA} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r³ instances of R^{TC}. In some embodiments, R^{TA} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r³ instances of R^{TC}.

In some embodiments, R^{TA} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r³ instances of R^{TC}. In some embodiments, R^{TA} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r³ instances of R^{TC} In some embodiments, R^{TA} is a C₁₋₆ aliphatic chain, phenyl, or a 3-7 membered saturated or partially unsaturated monocyclic carbocyclic ring; each of which is substituted by r³ instances of R^{TC}.

In some embodiments, R^{TA} is selected from the groups depicted in the compounds in Table 1.

As defined generally above, each R^{L} is independently R^{A} or R^{B} substituted by r⁴ instances of R^{LC}. In some embodiments, each R^{L} is independently R^{A}. In some embodiments, each R^{L} is independently R^{B} substituted by r⁴ instances of R^{LC}.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or deuterium.

In some embodiments, R^{L} is oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is oxo. In some embodiments, R^{L} is halogen. In some embodiments, R^{L} is —CN. In some embodiments, R^{L} is —NO₂. In some embodiments, R^{L} is —OR. In some embodiments, R^{L} is —SR. In some embodiments, R^{L} is —NR₂. In some embodiments, R^{L} is —S(O)₂R. In some embodiments, R^{L} is —S(O)₂NR₂. In some embodiments, R^{L} is —S(O)₂F. In some embodiments, R^{L} is —S(O)R. In some embodiments, R^{L} is —S(O)NR₂. In some embodiments, R^{L} is —S(O)(NR)R. In some embodiments, R^{L} is —C(O)R. In some embodiments, R^{L} is —C(O)OR. In some embodiments, R^{L} is —C(O)NR₂. In some embodiments, R^{L} is —C(O)N(R)OR. In some embodiments, R^{L} is —OC(O)R. In some embodiments, R^{L} is —OC(O)NR₂. In some embodiments, R^{L} is —N(R)C(O)OR. In some embodiments, R^{L} is —N(R)C(O)R. In some embodiments, R^{L} is —N(R)C(O)NR₂. In some embodiments, R^{L} is —N(R)C(NR)NR₂. In some embodiments, R^{L} is —N(R)S(O)₂NR₂. In some embodiments, R^{L} is —N(R)S(O)₂R. In some embodiments, R^{L} is —P(O)R₂. In some embodiments, R^{L} is —P(O)(R)OR. In some embodiments, R^{L} is —B(OR)₂. In some embodiments, R^{L} is deuterium.

In some embodiments, R^{L} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{L} is halogen, —CN, or —NO₂. In some embodiments, R^{L} is —OR, —SR, or —NR₂. In some embodiments, R^{L} is —S(O)₂R, —S(O)₂NR₂, —S(O)₂F, —S(O)R, —S(O)NR₂, or —S(O)(NR)R. In some embodiments, R^{L} is —C(O)R, —C(O)OR, —C(O)NR₂, or —C(O)N(R)OR. In some embodiments, R^{L} is —OC(O)R or —OC(O)NR₂. In some embodiments, R^{L} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{L} is —P(O)R₂ or —P(O)(R)OR.

In some embodiments, R^{L} is —OR, —OC(O)R, or —OC(O)NR₂. In some embodiments, R^{L} is —SR, —S(O)₂R, —S(O)₂NR₂, —S(O)₂F, —S(O)R, —S(O)NR₂, or —S(O)(NR)R. In some embodiments, R^{L} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{L} is —S(O)₂R, —S(O)₂NR₂, or —S(O)₂F. In some embodiments, R^{L} is —S(O)R, —S(O)NR₂, or —S(O)(NR)R. In some embodiments, R^{L} is —SR, —S(O)₂R, or —S(O)R. In some embodiments, R^{L} is —S(O)₂NR₂, —S(O)NR₂, or —S(O)(NR)R. In some embodiments, R^{L} is —S(O)₂NR₂ or —S(O)NR₂. In some embodiments, R^{L} is —SR, —S(O)₂R, —S(O)₂NR₂, or —S(O)R.

In some embodiments, R^{L} is —N(R)C(O)OR, —N(R)C(O)R, or —N(R)C(O)NR₂. In some embodiments, R^{L} is —N(R)S(O)₂NR₂ or —N(R)S(O)₂R. In some embodiments, R^{L} is —N(R)C(O)OR or —N(R)C(O)R. In some embodiments, R^{L} is —N(R)C(O)NR₂ or —N(R)S(O)₂NR₂. In some embodiments, R^{L} is —N(R)C(O)OR, —N(R)C(O)R, or —N(R)S(O)₂R.

In some embodiments, R^{L} is —NR₂, —N(R)C(O)OR, —N(R)C(O)R, or —N(R)C(O)NR₂. In some embodiments, R^{L} is —NR₂, —N(R)C(O)OR, or —N(R)C(O)R. In some embodiments, R^{L} is —NR₂, —N(R)C(O)OR, —N(R)C(O)R, or —N(R)S(O)₂R.

In some embodiments, R^{L} is a C₁₋₆ aliphatic chain; phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is a C₁₋₆ aliphatic chain substituted by r⁴ instances of R^{LC}. In some embodiments, R^{L} is phenyl substituted by r⁴ instances of R^{LC}. In some embodiments, R^{L} is naphthyl substituted by r⁴ instances of R^{LC}. In some embodiments, R^{L} is a 5-6 membered monocyclic heteroaryl ring having 1-4 heteroatoms independently selected from nitrogen, oxygen, and sulfur; wherein said ring is substituted by r⁴ instances of R^{LC}. In some embodiments, R^{L} is an 8-10 membered bicyclic heteroaryl ring having 1-4 heteroatoms independently selected from nitrogen, oxygen, and sulfur; wherein said ring is substituted by r⁴ instances of R^{LC}. In some embodiments, R^{L} is a 3-7 membered saturated or partially unsaturated monocyclic carbocyclic ring substituted by r instances of R^{LC}. In some embodiments, R^{L} is a 5-12 membered saturated or partially unsaturated bicyclic carbocyclic ring substituted by r⁴ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; wherein said ring is substituted by r⁴ instances of R^{LC}. In some embodiments, R^{L} is a 7-12 membered saturated or partially unsaturated bicyclic heterocyclic ring having 1-4 heteroatoms independently selected from nitrogen, oxygen, and sulfur; wherein said ring is substituted by r⁴ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or naphthyl; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring or a 5-12 membered saturated or partially unsaturated bicyclic carbocyclic ring; each of which is substituted by r⁴ instances of R^{LC}. In some embodiments, R^{L} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or a 5-6 membered monocyclic heteroaryl ring having 1-4 heteroatoms independently selected from nitrogen, oxygen, and sulfur; each of which is substituted by r⁴ instances of R^{LC}. In some embodiments, R^{L} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is naphthyl or an 8-10 membered bicyclic heteroaryl ring having 1-4 heteroatoms independently selected from nitrogen, oxygen, and sulfur; each of which is substituted by r⁴ instances of R^{LC}. In some embodiments, R^{L} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is phenyl or a 3-7 membered saturated or partially unsaturated monocyclic carbocyclic ring; each of which is substituted by r⁴ instances of R^{LC}. In some embodiments, R^{L} is naphthyl or a 5-12 membered saturated or partially unsaturated bicyclic carbocyclic ring; each of which is substituted by r⁴ instances of R^{LC}. In some embodiments, R^{L} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}. In some embodiments, R^{L} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}.

In some embodiments, R^{L} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur; each of which is substituted by r⁴ instances of R^{LC}. In some embodiments, R^{L} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring; each of which is substituted by r⁴ instances of R^{LC}. In some embodiments, R^{L} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur; each of which is substituted by r⁴ instances of R^{LC}.

In some embodiments, R^{L} is a C₁₋₆ aliphatic chain, a 3-7 membered saturated or partially unsaturated monocyclic carbocyclic ring, or a 5-12 membered saturated or partially unsaturated bicyclic carbocyclic ring; each of which is substituted by r⁴ instances of R^{LC} In some embodiments, R^{L} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur; each of which is substituted by r⁴ instances of R^{LC} In some embodiments, R^{L} is a C₁₋₆ aliphatic chain, phenyl, or a 3-7 membered saturated or partially unsaturated monocyclic carbocyclic ring; each of which is substituted by r⁴ instances of R^{LC}.

In some embodiments, R^{L} is selected from the groups depicted in the compounds in Table 1.

As defined generally above, each instance of R^{A} is independently oxo, deuterium, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{A} is independently oxo, halogen, —CN, —NO₂, —OR, —SF₅, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is oxo. In some embodiments, R^{A} is halogen. In some embodiments, R^{A} is —CN. In some embodiments, R^{A} is —NO₂. In some embodiments, R^{A} is —OR. In some embodiments, R^{A} is —SF₅. In some embodiments, R^{A} is —SR. In some embodiments, R^{A} is —NR₂. In some embodiments, R^{A} is —S(O)₂R. In some embodiments, R^{A} is —S(O)₂NR₂. In some embodiments, R^{A} is —S(O)₂F. In some embodiments, R^{A} is —S(O)R. In some embodiments, R^{A} is —S(O)NR₂. In some embodiments, R^{A} is —S(O)(NR)R. In some embodiments, R^{A} is —C(O)R. In some embodiments, R^{A} is —C(O)OR. In some embodiments, R^{A} is —C(O)NR₂. In some embodiments, R^{A} is —C(O)N(R)OR. In some embodiments, R^{A} is —OC(O)R. In some embodiments, R^{A} is —OC(O)NR₂. In some embodiments, R^{A} is —N(R)C(O)OR. In some embodiments, R^{A} is —N(R)C(O)R. In some embodiments, R^{A} is —N(R)C(O)NR₂. In some embodiments, R^{A} is —N(R)C(NR)NR₂. In some embodiments, R^{A} is —N(R)S(O)₂NR₂. In some embodiments, R^{A} is —N(R)S(O)₂R. In some embodiments, R^{A} is —P(O)R₂. In some embodiments, R^{A} is —P(O)(R)OR. In some embodiments, R^{A} is —B(OR)₂. In some embodiments, R^{A} is deuterium.

In some embodiments, R^{A} is halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, R^{A} is halogen, —CN, or —NO₂. In some embodiments, R^{A} is —OR, —SR, or —NR₂. In some embodiments, R^{A} is —S(O)₂R, —S(O)₂NR₂, —S(O)₂F, —S(O)R, —S(O)NR₂, or —S(O)(NR)R. In some embodiments, R^{A} is —C(O)R, —C(O)OR, —C(O)NR₂, or —C(O)N(R)OR. In some embodiments, R^{A} is —OC(O)R or —OC(O)NR₂. In some embodiments, R^{A} is —N(R)C(O)OR, —N(R)C(O)R, —N(R)C(O)NR₂, —N(R)C(NR)NR₂, —N(R)S(O)₂NR₂, or —N(R)S(O)₂R. In some embodiments, R^{A} is —P(O)R₂ or —P(O)(R)OR.

In some embodiments, R^{A} is —OR, —OC(O)R, or —OC(O)NR₂. In some embodiments, R^{A} is —SR, —S(O)₂R, —S(O)₂NR₂, —S(O)₂F, —S(O)R, —S(O)NR₂, or —S(O)(NR)R. In some embodiments, R^{A} is —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, R^{A} is —S(O)₂R, —S(O)₂NR₂, or —S(O)₂F. In some embodiments, R^{A} is —S(O)R, —S(O)NR₂, or —S(O)(NR)R. In some embodiments, R^{A} is —SR, —S(O)₂R, or —S(O)R. In some embodiments, R^{A} is —S(O)₂NR₂, —S(O)NR₂, or —S(O)(NR)R. In some embodiments, R^{A} is —S(O)₂NR₂ or —S(O)NR₂. In some embodiments, R^{A} is —SR, —S(O)₂R, —S(O)₂NR₂, or —S(O)R.

In some embodiments, R^{A} is —N(R)C(O)OR, —N(R)C(O)R, or —N(R)C(O)NR₂. In some embodiments, R^{A} is —N(R)S(O)₂NR₂ or —N(R)S(O)₂R. In some embodiments, RA is —N(R)C(O)OR or —N(R)C(O)R. In some embodiments, R^{A} is —N(R)C(O)NR₂ or —N(R)S(O)₂NR₂. In some embodiments, R^{A} is —N(R)C(O)OR, —N(R)C(O)R, or —N(R)S(O)₂R.

In some embodiments, R^{A} is —NR₂, —N(R)C(O)OR, —N(R)C(O)R, or —N(R)C(O)NR₂. In some embodiments, R^{A} is —NR₂, —N(R)C(O)OR, or —N(R)C(O)R. In some embodiments, R^{A} is —NR₂, —N(R)C(O)OR, —N(R)C(O)R, or —N(R)S(O)₂R.

In some embodiments, R^{A} is selected from the groups depicted in the compounds in Table 1.

As defined generally above, each instance of R^{B} is independently a C₁₋₆ aliphatic chain; phenyl; naphthyl; cubanyl; adamantyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain. In some embodiments, R^{B} is phenyl. In some embodiments, R^{B} is naphthyl. In some embodiments, R^{B} is cubanyl. In some embodiments, R^{B} is adamantyl. In some embodiments, R^{B} is a 5-6 membered monocyclic heteroaryl ring having 1-4 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring. In some embodiments, R^{B} is a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur. In some embodiments, R^{B} is a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; an 8-10 membered bicyclic heteroaryl ring having 1-4 heteroatoms independently selected from nitrogen, oxygen, and sulfur; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur. In some embodiments, R^{B} is naphthyl; an 8-10 membered bicyclic heteroaryl ring having 1-4 heteroatoms independently selected from nitrogen, oxygen, and sulfur; a 5-12 membered saturated or partially unsaturated bicyclic carbocyclic ring; or a 7-12 membered saturated or partially unsaturated bicyclic heterocyclic ring having 1-4 heteroatoms independently selected from nitrogen, oxygen, and sulfur.

In some embodiments, R^{B} is phenyl or naphthyl. In some embodiments, R^{B} is a 5-6 membered monocyclic heteroaryl ring having 1-4 heteroatoms independently selected from nitrogen, oxygen, and sulfur; or an 8-10 membered bicyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 5-12 membered saturated or partially unsaturated bicyclic carbocyclic ring. In some embodiments, R^{B} is a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is phenyl or a 5-6 membered monocyclic heteroaryl ring having 1-4 heteroatoms independently selected from nitrogen, oxygen, and sulfur. In some embodiments, R^{B} is a 3-7 membered saturated or partially unsaturated monocyclic carbocyclic ring or a 3-7 membered saturated or partially unsaturated monocyclic heterocyclic ring having 1-2 heteroatoms independently selected from nitrogen, oxygen, and sulfur. In some embodiments, R^{B} is naphthyl or an 8-10 membered bicyclic heteroaryl ring having 1-4 heteroatoms independently selected from nitrogen, oxygen, and sulfur. In some embodiments, R^{B} is a 5-12 membered saturated or partially unsaturated bicyclic carbocyclic ring or a 7-12 membered saturated or partially unsaturated bicyclic heterocyclic ring having 1-4 heteroatoms independently selected from nitrogen, oxygen, and sulfur.

In some embodiments, R^{B} is phenyl or a 3-7 membered saturated or partially unsaturated monocyclic carbocyclic ring. In some embodiments, R^{B} is naphthyl or a 5-12 membered saturated or partially unsaturated bicyclic carbocyclic ring. In some embodiments, R^{B} is a 5-6 membered monocyclic heteroaryl ring having 1-4 heteroatoms independently selected from nitrogen, oxygen, and sulfur; or a 3-7 membered saturated or partially unsaturated monocyclic heterocyclic ring having 1-2 heteroatoms independently selected from nitrogen, oxygen, and sulfur. In some embodiments, R^{B} is an 8-10 membered bicyclic heteroaryl ring having 1-4 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur.

In some embodiments, R^{B} is a C₁₋₆ aliphatic chain; a 3-7 membered saturated or partially unsaturated monocyclic carbocyclic ring; a 5-12 membered saturated or partially unsaturated bicyclic carbocyclic ring; a 3-7 membered saturated or partially unsaturated monocyclic heterocyclic ring having 1-2 heteroatoms independently selected from nitrogen, oxygen, and sulfur; or a 7-12 membered saturated or partially unsaturated bicyclic heterocyclic ring having 1-4 heteroatoms independently selected from nitrogen, oxygen, and sulfur. In some embodiments, R^{B} is a C₁₋₆ aliphatic chain; phenyl; naphthyl; a 3-7 membered saturated or partially unsaturated monocyclic carbocyclic ring; or a 5-12 membered saturated or partially unsaturated bicyclic carbocyclic ring. In some embodiments, R^{B} is a C₁₋₆ aliphatic chain; phenyl; a 5-6 membered monocyclic heteroaryl ring having 1-4 heteroatoms independently selected from nitrogen, oxygen, and sulfur; a 3-7 membered saturated or partially unsaturated monocyclic carbocyclic ring; or a 3-7 membered saturated or partially unsaturated monocyclic heterocyclic ring having 1-2 heteroatoms independently selected from nitrogen, oxygen, and sulfur.

In some embodiments, R^{B} is a C₁₋₆ aliphatic chain, a 3-7 membered saturated or partially unsaturated monocyclic carbocyclic ring, or a 5-12 membered saturated or partially unsaturated bicyclic carbocyclic ring. In some embodiments, R^{B} is a C₁₋₆ aliphatic chain, a 3-7 membered saturated or partially unsaturated monocyclic carbocyclic ring, or a 3-7 membered saturated or partially unsaturated monocyclic heterocyclic ring having 1-2 heteroatoms independently selected from nitrogen, oxygen, and sulfur. In some embodiments, R^{B} is a C₁₋₆ aliphatic chain, phenyl, or a 3-7 membered saturated or partially unsaturated monocyclic carbocyclic ring.

In some embodiments, R^{B} is selected from the groups depicted in the compounds in Table 1.

As defined generally above, each instance of R^{1C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{1C} is oxo. In some embodiments, R^{1C} is deuterium. In some embodiments, each instance of R^{1C} is independently halogen. In some embodiments, R^{1C} is —CN. In some embodiments, R^{1C} is —NO₂. In some embodiments, R^{1C} is —OR. In some embodiments, R^{1C} is —SR. In some embodiments, R^{1C} is —NR₂. In some embodiments, R^{1C} is —S(O)₂R. In some embodiments, R^{1C} is —S(O)₂NR₂. In some embodiments, R^{1C} is —S(O)₂F. In some embodiments, R^{1C} is —S(O)R. In some embodiments, R^{1C} is —S(O)NR₂. In some embodiments, R^{1C} is —S(O)(NR)R. In some embodiments, R^{1C} is —C(O)R. In some embodiments, R^{1C} is —C(O)OR. In some embodiments, R^{1C} is —C(O)NR₂. In some embodiments, R^{1C} is —C(O)N(R)OR. In some embodiments, R^{1C} is —OC(O)R. In some embodiments, R^{1C} is —OC(O)NR₂. In some embodiments, R^{1C} is —N(R)C(O)OR. In some embodiments, R^{1C} is —N(R)C(O)R. In some embodiments, R^{1C} is —N(R)C(O)NR₂. In some embodiments, R^{1C} is —N(R)C(NR)NR₂. In some embodiments, R^{1C} is —N(R)S(O)₂NR₂. In some embodiments, R^{1C} is —N(R)S(O)₂R. In some embodiments, R^{1C} is —P(O)R₂. In some embodiments, R^{1C} is —P(O)(R)OR. In some embodiments, R^{1C} is —B(OR)₂.

In some embodiments, each instance of R^{1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{1C} is independently halogen, —CN, or —NO₂. In some embodiments, each instance of R^{1C} is independently —OR, —SR, or —NR₂. In some embodiments, each instance of R^{1C} is independently —S(O)₂R, —S(O)₂NR₂, —S(O)₂F, —S(O)R, —S(O)NR₂, or —S(O)(NR)R. In some embodiments, each instance of R^{1C} is independently —C(O)R, —C(O)OR, —C(O)NR₂, or —C(O)N(R)OR. In some embodiments, each instance of R^{1C} is independently —OC(O)R or —OC(O)NR₂. In some embodiments, each instance of R^{1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{1C} is independently —P(O)R₂ or —P(O)(R)OR.

In some embodiments, each instance of R^{1C} is independently —OR, —OC(O)R, or —OC(O)NR₂. In some embodiments, each instance of R^{1C} is independently —SR, —S(O)₂R, —S(O)₂NR₂, —S(O)₂F, —S(O)R, —S(O)NR₂, or —S(O)(NR)R. In some embodiments, each instance of R^{1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{1C} is independently —S(O)₂R, —S(O)₂NR₂, or —S(O)₂F. In some embodiments, each instance of R^{1C} is independently —S(O)R, —S(O)NR₂, or —S(O)(NR)R. In some embodiments, each instance of R^{1C} is independently —SR, —S(O)₂R, or —S(O)R. In some embodiments, each instance of R^{1C} is independently —S(O)₂NR₂, —S(O)NR₂, or —S(O)(NR)R. In some embodiments, each instance of R^{1C} is independently —S(O)₂NR₂ or —S(O)NR₂. In some embodiments, each instance of R^{1C} is independently —SR, —S(O)₂R, —S(O)₂NR₂, or —S(O)R.

In some embodiments, each instance of R^{1C} is independently —N(R)C(O)OR, —N(R)C(O)R, or —N(R)C(O)NR₂. In some embodiments, each instance of R^{1C} is independently —N(R)S(O)₂NR₂ or —N(R)S(O)₂R. In some embodiments, each instance of R^{1C} is independently —N(R)C(O)OR or —N(R)C(O)R. In some embodiments, each instance of R^{1C} is independently —N(R)C(O)NR₂ or —N(R)S(O)₂NR₂. In some embodiments, each instance of Ric is independently —N(R)C(O)OR, —N(R)C(O)R, or —N(R)S(O)₂R.

In some embodiments, each instance of R^{1C} is independently —NR₂, —N(R)C(O)OR, —N(R)C(O)R, or —N(R)C(O)NR₂. In some embodiments, each instance of R^{1C} is independently —NR₂, —N(R)C(O)OR, or —N(R)C(O)R. In some embodiments, each instance of Ric is independently —NR₂, —N(R)C(O)OR, —N(R)C(O)R, or —N(R)S(O)₂R.

In some embodiments, each instance of R^{1C} is independently an optionally substituted C₁₋₆ aliphatic. In some embodiments, each instance of R^{1C} is independently an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted C₁₋₆ aliphatic or an optionally substituted phenyl. In some embodiments, each instance of R^{1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic. In some embodiments, R^{1C} is phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur. In some embodiments, each instance of R^{1C} is independently a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{1C} is independently phenyl or a 5-6 membered monocyclic heteroaryl ring having 1-4 heteroatoms independently selected from nitrogen, oxygen, and sulfur.

In some embodiments, each instance of R^{1C} is independently a C₁₋₆ aliphatic or phenyl. In some embodiments, each instance of R^{1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of RC is independently halogen, —CN, —O-(optionally substituted C₁₋₆ aliphatic), or an optionally substituted C₁₋₆ aliphatic. In some embodiments, each instance of R^{1C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{1C} is independently halogen or C₁₋₃ aliphatic optionally substituted with 1-3 halogen. In some embodiments, each instance of R^{1C} is independently fluorine, chlorine, —CH₃, —CHF₂, or —CF₃.

In some embodiments, each instance of R^{1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{1C} is independently selected from the groups depicted in the compounds in Table 1.

As defined generally above, each instance of R^{2C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{2C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{2C} is oxo. In some embodiments, R^{2C} is deuterium. In some embodiments, each instance of R^{2C} is independently halogen. In some embodiments, R^{2C} is —CN. In some embodiments, R^{2C} is —NO₂. In some embodiments, R^{2C} is —OR. In some embodiments, R^{2C} is —SR. In some embodiments, R^{2C} is —NR₂. In some embodiments, R^{2C} is —S(O)₂R. In some embodiments, R^{2C} is —S(O)₂NR₂. In some embodiments, R^{2C} is —S(O)₂F. In some embodiments, R^{2C} is —S(O)R. In some embodiments, R^{2C} is —S(O)NR₂. In some embodiments, R^{2C} is —S(O)(NR)R. In some embodiments, R^{2C} is —C(O)R. In some embodiments, R^{2C} is —C(O)OR. In some embodiments, R^{2C} is —C(O)NR₂. In some embodiments, R^{2C} is —C(O)N(R)OR. In some embodiments, R^{2C} is —OC(O)R. In some embodiments, R^{2C} is —OC(O)NR₂. In some embodiments, R^{2C} is —N(R)C(O)OR. In some embodiments, R^{2C} is —N(R)C(O)R. In some embodiments, R^{2C} is —N(R)C(O)NR₂. In some embodiments, R^{2C} is —N(R)C(NR)NR₂. In some embodiments, R^{2C} is —N(R)S(O)₂NR₂. In some embodiments, R^{2C} is —N(R)S(O)₂R. In some embodiments, R^{2C} is —P(O)R₂. In some embodiments, R^{2C} is —P(O)(R)OR. In some embodiments, R^{2C} is —B(OR)₂.

In some embodiments, each instance of R^{2C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{2C} is independently halogen, —CN, or —NO₂. In some embodiments, each instance of R^{2C} is independently —OR, —SR, or —NR₂. In some embodiments, each instance of R^{2C} is independently —S(O)₂R, —S(O)₂NR₂, —S(O)₂F, —S(O)R, —S(O)NR₂, or —S(O)(NR)R. In some embodiments, each instance of R^{2C} is independently —C(O)R, —C(O)OR, —C(O)NR₂, or —C(O)N(R)OR. In some embodiments, each instance of R^{2C} is independently —OC(O)R or —OC(O)NR₂. In some embodiments, each instance of R^{2C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{2C} is independently —P(O)R₂ or —P(O)(R)OR.

In some embodiments, each instance of R^{2C} is independently —OR, —OC(O)R, or —OC(O)NR₂. In some embodiments, each instance of R^{2C} is independently —SR, —S(O)₂R, —S(O)₂NR₂, —S(O)₂F, —S(O)R, —S(O)NR₂, or —S(O)(NR)R. In some embodiments, each instance of R^{2C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{2C} is independently —S(O)₂R, —S(O)₂NR₂, or —S(O)₂F. In some embodiments, each instance of R^{2C} is independently —S(O)R, —S(O)NR₂, or —S(O)(NR)R. In some embodiments, each instance of R^{2C} is independently —SR, —S(O)₂R, or —S(O)R. In some embodiments, each instance of R^{2C} is independently —S(O)₂NR₂, —S(O)NR₂, or —S(O)(NR)R. In some embodiments, each instance of R^{2C} is independently —S(O)₂NR₂ or —S(O)NR₂. In some embodiments, each instance of R^{2C} is independently —SR, —S(O)₂R, —S(O)₂NR₂, or —S(O)R.

In some embodiments, each instance of R^{2C} is independently —N(R)C(O)OR, —N(R)C(O)R, or —N(R)C(O)NR₂. In some embodiments, each instance of R^{2C} is independently —N(R)S(O)₂NR₂ or —N(R)S(O)₂R. In some embodiments, each instance of R^{2C} is independently —N(R)C(O)OR or —N(R)C(O)R. In some embodiments, each instance of R^{2C} is independently —N(R)C(O)NR₂ or —N(R)S(O)₂NR₂. In some embodiments, each instance of R^{2C} is independently —N(R)C(O)OR, —N(R)C(O)R, or —N(R)S(O)₂R.

In some embodiments, each instance of R^{2C} is independently —NR₂, —N(R)C(O)OR, —N(R)C(O)R, or —N(R)C(O)NR₂. In some embodiments, each instance of R^{2C} is independently —NR₂, —N(R)C(O)OR, or —N(R)C(O)R. In some embodiments, each instance of R^{2C} is independently —NR₂, —N(R)C(O)OR, —N(R)C(O)R, or —N(R)S(O)₂R.

In some embodiments, each instance of R^{2C} is independently an optionally substituted C₁₋₆ aliphatic. In some embodiments, each instance of R^{2C} is independently an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{2C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted C₁₋₆ aliphatic or an optionally substituted phenyl. In some embodiments, each instance of R^{2C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{2C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic. In some embodiments, R^{2C} is phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur. In some embodiments, each instance of R^{2C} is independently a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{2C} is independently phenyl or a 5-6 membered monocyclic heteroaryl ring having 1-4 heteroatoms independently selected from nitrogen, oxygen, and sulfur.

In some embodiments, each instance of R^{2C} is independently a C₁₋₆ aliphatic or phenyl. In some embodiments, each instance of R^{2C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{2C} is independently halogen, —CN, —O-(optionally substituted C₁₋₆ aliphatic), or an optionally substituted C₁₋₆ aliphatic. In some embodiments, each instance of R^{2C} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{2C} is independently halogen or C₁₋₃ aliphatic optionally substituted with 1-3 halogen. In some embodiments, each instance of R^{2C} is independently fluorine, chlorine, —CH₃, —CHF₂, or —CF₃.

In some embodiments, each instance of R^{2C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{2C} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{2C} is independently a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 halogen atoms. In some embodiments, each instance of R^{2C} is independently a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{2C} is independently a C₁₋₆ aliphatic optionally substituted with 1, 2, or 3 atoms independently selected from halogen and deuterium. In some embodiments, each instance of R^{2C} is independently a C₁₋₆ aliphatic optionally substituted with 1, 2, or 3 atoms independently selected from halogen.

In some embodiments, each instance of R^{2C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{2C} is independently oxo, deuterium, halogen, or —CN. In some embodiments, each instance of R^{2C} is independently oxo, —OH, —O—(C₁₋₃ aliphatic), or C₁₋₃ aliphatic, wherein each C₁₋₃ aliphatic is optionally substituted with one or more halogen atoms. In some embodiments, each instance of R^{2C} is independently —O—(C₁₋₃ aliphatic) or C₁₋₃ aliphatic, wherein each C₁₋₃ aliphatic is optionally substituted with one or more halogen atoms. In some embodiments, each instance of R^{2C} is independently —O—(C₁₋₃ aliphatic) or C₁₋₃ aliphatic.

In some embodiments, each instance of R^{2C} is independently selected from the groups depicted in the compounds in Table 1.

As defined generally above, each instance of R^{TC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{TC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TC} is oxo. In some embodiments, R^{TC} is deuterium. In some embodiments, each instance of R^{TC} is independently halogen. In some embodiments, R^{TC} is —CN. In some embodiments, R^{TC} is —NO₂. In some embodiments, R^{TC} is —OR. In some embodiments, R^{TC} is —SR. In some embodiments, R^{TC} is —NR₂. In some embodiments, R^{TC} is —S(O)₂R. In some embodiments, R^{TC} is —S(O)₂NR₂. In some embodiments, R^{TC} is —S(O)₂F. In some embodiments, R^{TC} is —S(O)R. In some embodiments, R^{TC} is —S(O)NR₂. In some embodiments, R^{TC} is —S(O)(NR)R. In some embodiments, R^{TC} is —C(O)R. In some embodiments, R^{TC} is —C(O)OR. In some embodiments, R^{TC} is —C(O)NR₂. In some embodiments, R^{TC} is —C(O)N(R)OR. In some embodiments, R^{TC} is —OC(O)R. In some embodiments, R^{TC} is —OC(O)NR₂. In some embodiments, R^{TC} is —N(R)C(O)OR. In some embodiments, R^{TC} is —N(R)C(O)R. In some embodiments, R^{TC} is —N(R)C(O)NR₂. In some embodiments, R^{TC} is —N(R)C(NR)NR₂. In some embodiments, R^{TC} is —N(R)S(O)₂NR₂. In some embodiments, R^{TC} is —N(R)S(O)₂R. In some embodiments, R^{TC} is —P(O)R₂. In some embodiments, R^{TC} is —P(O)(R)OR. In some embodiments, R^{TC} is —B(OR)₂.

In some embodiments, each instance of R^{TC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TC} is independently halogen, —CN, or —NO₂. In some embodiments, each instance of R^{TC} is independently —OR, —SR, or —NR₂. In some embodiments, each instance of R^{TC} is independently —S(O)₂R, —S(O)₂NR₂, —S(O)₂F, —S(O)R, —S(O)NR₂, or —S(O)(NR)R. In some embodiments, each instance of R^{TC} is independently —C(O)R, —C(O)OR, —C(O)NR₂, or —C(O)N(R)OR. In some embodiments, each instance of R^{TC} is independently —OC(O)R or —OC(O)NR₂. In some embodiments, each instance of R^{TC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TC} is independently —P(O)R₂ or —P(O)(R)OR.

In some embodiments, each instance of R^{TC} is independently —OR, —OC(O)R, or —OC(O)NR₂. In some embodiments, each instance of R^{TC} is independently —SR, —S(O)₂R, —S(O)₂NR₂, —S(O)₂F, —S(O)R, —S(O)NR₂, or —S(O)(NR)R. In some embodiments, each instance of R^{TC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TC} is independently —S(O)₂R, —S(O)₂NR₂, or —S(O)₂F. In some embodiments, each instance of R^{TC} is independently —S(O)R, —S(O)NR₂, or —S(O)(NR)R. In some embodiments, each instance of R^{TC} is independently —SR, —S(O)₂R, or —S(O)R. In some embodiments, each instance of R^{TC} is independently —S(O)₂NR₂, —S(O)NR₂, or —S(O)(NR)R. In some embodiments, each instance of R^{TC} is independently —S(O)₂NR₂ or —S(O)NR₂. In some embodiments, each instance of R^{TC} is independently —SR, —S(O)₂R, —S(O)₂NR₂, or —S(O)R.

In some embodiments, each instance of R^{TC} is independently —N(R)C(O)OR, —N(R)C(O)R, or —N(R)C(O)NR₂. In some embodiments, each instance of R^{TC} is independently —N(R)S(O)₂NR₂ or —N(R)S(O)₂R. In some embodiments, each instance of R^{TC} is independently —N(R)C(O)OR or —N(R)C(O)R. In some embodiments, each instance of R^{TC} is independently —N(R)C(O)NR₂ or —N(R)S(O)₂NR₂. In some embodiments, each instance of R^{TC} is independently —N(R)C(O)OR, —N(R)C(O)R, or —N(R)S(O)₂R.

In some embodiments, each instance of R^{TC} is independently —NR₂, —N(R)C(O)OR, —N(R)C(O)R, or —N(R)C(O)NR₂. In some embodiments, each instance of R^{TC} is independently —NR₂, —N(R)C(O)OR, or —N(R)C(O)R. In some embodiments, each instance of R^{TC} is independently —NR₂, —N(R)C(O)OR, —N(R)C(O)R, or —N(R)S(O)₂R.

In some embodiments, each instance of R^{TC} is independently an optionally substituted C₁₋₆ aliphatic. In some embodiments, each instance of R^{TC} is independently an optionally substituted phenyl. In some embodiments, each instance of R^{TC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TC} is independently an optionally substituted 5-6 membered monocyclic heteroaryl ring having 1-4 heteroatoms independently selected from nitrogen, oxygen, and sulfur.

In some embodiments, each instance of R^{TC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TC} is independently an optionally substituted C₁₋₆ aliphatic or an optionally substituted phenyl. In some embodiments, each instance of R^{TC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic. In some embodiments, R^{TC} is phenyl. In some embodiments, each instance of R^{TC} is independently a 3-7 membered saturated or partially unsaturated monocyclic heterocyclic ring having 1-2 heteroatoms independently selected from nitrogen, oxygen, and sulfur. In some embodiments, each instance of R^{TC} is independently a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TC} is independently phenyl or a 5-6 membered monocyclic heteroaryl ring having 1-4 heteroatoms independently selected from nitrogen, oxygen, and sulfur.

In some embodiments, each instance of R^{TC} is independently a C₁₋₆ aliphatic or phenyl. In some embodiments, each instance of R^{TC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TC} is independently halogen, —CN, —O-(optionally substituted C₁₋₆ aliphatic), or an optionally substituted C₁₋₆ aliphatic. In some embodiments, each instance of R^{TC} is independently halogen, —CN, —O—(C₁₋₆ aliphatic), or C₁₋₆ aliphatic; wherein each C₁₋₆ aliphatic is optionally substituted with one or more halogen atoms. In some embodiments, each instance of R^{TC} is independently halogen or C₁₋₃ aliphatic optionally substituted with 1-3 halogen. In some embodiments, each instance of R^{TC} is independently fluorine, chlorine, —CH₃, —CHF₂, or —CF₃.

In some embodiments, each instance of R^{TC} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 atoms independently selected from halogen and deuterium. In some embodiments, each instance of R^{TC} is a C₁₋₆ aliphatic optionally substituted with (i) 1 or 2 groups independently selected from —O—(C₁₋₆ aliphatic), —OH, —N(C₁₋₆ aliphatic)₂, and —CN, and (ii) 1, 2, or 3 halogen atoms. In some embodiments, each instance of R^{TC} is a C₁₋₆ aliphatic optionally substituted with 1 or 2 groups independently selected from —O—(C₁₋₆ aliphatic), —OH, —N(C₁₋₆ aliphatic)₂, and —CN. In some embodiments, each instance of R^{TC} is a C₁₋₆ aliphatic optionally substituted with 1, 2, or 3 atoms independently selected from halogen and deuterium. In some embodiments, each instance of R^{TC} is a C₁₋₆ aliphatic optionally substituted with 1, 2, or 3 atoms independently selected from halogen.

In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{T}c is independently oxo, deuterium, halogen, or —CN. In some embodiments, each instance of R^{TC} is independently oxo, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently —O—(C₁₋₃ aliphatic) or C₁₋₃ aliphatic, wherein each C₁₋₃ aliphatic is optionally substituted with one or more halogen atoms. In some embodiments, each instance of R^{TC} is is independently —O—(C₁₋₃ aliphatic) or C₁₋₃ aliphatic.

In some embodiments, each instance of R^{TC} is independently halogen, —CN, —OH, —O-(optionally substituted C₁₋₃ aliphatic), or an optionally substituted C₁₋₃ aliphatic. In some embodiments, each instance of R^{TC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently fluorine, chlorine, —OH, —OCH₃, —OCF₃, —CH₃, —CHF₂, or —CF₃. In some embodiments, each instance of R^{TC} is independently fluorine or —OH.

In some embodiments, each instance of R^{TC} is independently oxo, deuterium, halogen, —CN, —OH, —O-(optionally substituted C₁₋₃ aliphatic), or an optionally substituted C₁₋₃ aliphatic. In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently oxo, deuterium, fluorine, chlorine, —CN, —OH, —OCH₃, —OCF₃, —CH₃, —CHF₂, or —CF₃. In some embodiments, each instance of R^{TC} is independently oxo, deuterium, —CN, fluorine, or —OH. In some embodiments, each instance of R^{TC} is independently oxo, deuterium, —CN, —CH₃, or —CHF₂. In some embodiments, each instance of R^{TC} is independently deuterium, —CN, —CH₃, or —CHF₂.

In some embodiments, each instance of R^{TC} is independently oxo, halogen, —CN, —OH, —O-(optionally substituted C₁₋₃ aliphatic), or an optionally substituted C₁₋₃ aliphatic. In some embodiments, each instance of R^{TC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TC} is independently oxo, fluorine, chlorine, —CN, —OH, —OCH₃, —OCF₃, —CH₃, —CHF₂, or —CF₃. In some embodiments, each instance of R^{TC} is independently oxo, —CN, fluorine, or —OH. In some embodiments, each instance of R^{TC} is independently oxo, —CN, —CH₃, or —CHF₂. In some embodiments, each instance of R^{TC} is independently —CN, —CH₃, or —CHF₂.

In some embodiments, each instance of R^{TC} is independently selected from the groups depicted in the compounds in Table 1.

As defined generally above, each instance of R^{TTC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{TTC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TTC} is oxo. In some embodiments, R^{TTC} is deuterium. In some embodiments, each instance of R^{TTC} is independently halogen. In some embodiments, R^{TTC} is —CN. In some embodiments, R^{TTC} is —NO₂. In some embodiments, R^{TTC} is —OR. In some embodiments, R^{TTC} is —SR. In some embodiments, R^{TTC} is —NR₂. In some embodiments, R^{TTC} is —S(O)₂R. In some embodiments, R^{TTC} is —S(O)₂NR₂. In some embodiments, R^{TTC} is —S(O)₂F. In some embodiments, R^{TTC} is —S(O)R. In some embodiments, R^{TTC} is —S(O)NR₂. In some embodiments, R^{TTC} is —S(O)(NR)R. In some embodiments, R^{TTC} is —C(O)R. In some embodiments, R^{TTC} is —C(O)OR. In some embodiments, R^{TTC} is —C(O)NR₂. In some embodiments, R^{TTC} is —C(O)N(R)OR. In some embodiments, R^{TTC} is —OC(O)R. In some embodiments, R^{TTC} is —OC(O)NR₂. In some embodiments, R^{TTC} is —N(R)C(O)OR. In some embodiments, R^{TTC} is —N(R)C(O)R. In some embodiments, R^{TTC} is —N(R)C(O)NR₂. In some embodiments, R^{TTC} is —N(R)C(NR)NR₂. In some embodiments, R^{TTC} is —N(R)S(O)₂NR₂. In some embodiments, R^{TTC} is —N(R)S(O)₂R. In some embodiments, R^{TTC} is —P(O)R₂. In some embodiments, R^{TTC} is —P(O)(R)OR. In some embodiments, R^{TTC} is —B(OR)₂.

In some embodiments, each instance of R^{TTC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TTC} is independently halogen, —CN, or —NO₂. In some embodiments, each instance of R^{TTC} is independently —OR, —SR, or —NR₂. In some embodiments, each instance of R^{TTC} is independently —S(O)₂R, —S(O)₂NR₂, —S(O)₂F, —S(O)R, —S(O)NR₂, or —S(O)(NR)R. In some embodiments, each instance of R^{TTC} is independently —C(O)R, —C(O)OR, —C(O)NR₂, or —C(O)N(R)OR. In some embodiments, each instance of R^{TTC} is independently —OC(O)R or —OC(O)NR₂. In some embodiments, each instance of R^{TTC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TTC} is independently —P(O)R₂ or —P(O)(R)OR.

In some embodiments, each instance of R^{TTC} is independently —OR, —OC(O)R, or —OC(O)NR₂. In some embodiments, each instance of R^{TTC} is independently —SR, —S(O)₂R, —S(O)₂NR₂, —S(O)₂F, —S(O)R, —S(O)NR₂, or —S(O)(NR)R. In some embodiments, each instance of R^{TTC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TTC} is independently —S(O)₂R, —S(O)₂NR₂, or —S(O)₂F. In some embodiments, each instance of R^{TTC} is independently —S(O)R, —S(O)NR₂, or —S(O)(NR)R. In some embodiments, each instance of R^{TTC} is independently —SR, —S(O)₂R, or —S(O)R. In some embodiments, each instance of R^{TTC} is independently —S(O)₂NR₂, —S(O)NR₂, or —S(O)(NR)R. In some embodiments, each instance of R^{TTC} is independently —S(O)₂NR₂ or —S(O)NR₂. In some embodiments, each instance of R^{TTC} is independently —SR, —S(O)₂R, —S(O)₂NR₂, or —S(O)R.

In some embodiments, each instance of R^{TTC} is independently —N(R)C(O)OR, —N(R)C(O)R, or —N(R)C(O)NR₂. In some embodiments, each instance of R^{TTC} is independently —N(R)S(O)₂NR₂ or —N(R)S(O)₂R. In some embodiments, each instance of R^{TTC} is independently —N(R)C(O)OR or —N(R)C(O)R. In some embodiments, each instance of R^{TTC} is independently —N(R)C(O)NR₂ or —N(R)S(O)₂NR₂. In some embodiments, each instance of R^{TTC} is independently —N(R)C(O)OR, —N(R)C(O)R, or —N(R)S(O)₂R.

In some embodiments, each instance of R^{TTC} is independently —NR₂, —N(R)C(O)OR, —N(R)C(O)R, or —N(R)C(O)NR₂. In some embodiments, each instance of R^{TTC} is independently —NR₂, —N(R)C(O)OR, or —N(R)C(O)R. In some embodiments, each instance of R^{TTC} is independently —NR₂, —N(R)C(O)OR, —N(R)C(O)R, or —N(R)S(O)₂R.

In some embodiments, each instance of R^{TTC} is independently an optionally substituted C₁₋₆ aliphatic. In some embodiments, each instance of R^{TTC} is independently an optionally substituted phenyl. In some embodiments, each instance of R^{TTC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TTC} is independently an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TTC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted C₁₋₆ aliphatic or an optionally substituted phenyl. In some embodiments, each instance of R^{TTC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TTC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic. In some embodiments, R^{TTC} is phenyl. In some embodiments, each instance of R^{TTC} is independently a 3-7 membered saturated or partially unsaturated monocyclic heterocyclic ring having 1-2 heteroatoms independently selected from nitrogen, oxygen, and sulfur. In some embodiments, each instance of R^{TTC} is independently a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TTC} is independently phenyl or a 5-6 membered monocyclic heteroaryl ring having 1-4 heteroatoms independently selected from nitrogen, oxygen, and sulfur.

In some embodiments, each instance of R^{TTC} is independently a C₁₋₆ aliphatic or phenyl. In some embodiments, each instance of R^{TTC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TTC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TTC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{TTC} is independently halogen, —CN, —OH, —O-(optionally substituted C₁₋₃ aliphatic), or an optionally substituted C₁₋₃ aliphatic. In some embodiments, each instance of R^{TTC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently fluorine, chlorine, —OH, —OCH₃, —OCF₃, —CH₃, —CHF₂, or —CF₃. In some embodiments, each instance of R^{TTC} is independently fluorine or —OH.

In some embodiments, each instance of R^{TTC} is independently oxo, deuterium, halogen, —CN, —OH, —O-(optionally substituted C₁₋₃ aliphatic), or an optionally substituted C₁₋₃ aliphatic. In some embodiments, each instance of R^{TTC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TTC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently oxo, deuterium, fluorine, chlorine, —CN, —OH, —OCH₃, —OCF₃, —CH₃, —CHF₂, or —CF₃. In some embodiments, each instance of R^{TTC} is independently oxo, deuterium, —CN, fluorine, or —OH. In some embodiments, each instance of R^{TTC} is independently oxo, deuterium, —CN, —CH₃, or —CHF₂. In some embodiments, each instance of R^{TTC} is independently deuterium, —CN, —CH₃, or —CHF₂.

In some embodiments, each instance of R^{TTC} is independently oxo, halogen, —CN, —OH, —O-(optionally substituted C₁₋₃ aliphatic), or an optionally substituted C₁₋₃ aliphatic. In some embodiments, each instance of R^{TTC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TTC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TTC} is independently oxo, fluorine, chlorine, —CN, —OH, —OCH₃, —OCF₃, —CH₃, —CHF₂, or —CF₃. In some embodiments, each instance of R^{TTC} is independently oxo, —CN, fluorine, or —OH. In some embodiments, each instance of R^{TTC} is independently oxo, —CN, —CH₃, or —CHF₂. In some embodiments, each instance of R^{TTC} is independently —CN, —CH₃, or —CHF₂.

In some embodiments, each instance of R^{TTC} is independently selected from the groups depicted in the compounds in Table 1.

As defined generally above, each instance of R^{11C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{11C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{11C} is oxo. In some embodiments, R^{11C} is deuterium. In some embodiments, each instance of R^{11C} is independently halogen. In some embodiments, R^{11C} is —CN. In some embodiments, R^{11C} is —NO₂. In some embodiments, R^{11C} is —OR. In some embodiments, R^{11C} is —SR. In some embodiments, R^{11C} is —NR₂. In some embodiments, R^{11C} is —S(O)₂R. In some embodiments, R^{11C} is —S(O)₂NR₂. In some embodiments, R^{11C} is —S(O)₂F. In some embodiments, R^{11C} is —S(O)R. In some embodiments, R^{11C} is —S(O)NR₂. In some embodiments, R^{11C} is —S(O)(NR)R. In some embodiments, R^{11C} is —C(O)R. In some embodiments, R^{11C} is —C(O)OR. In some embodiments, R^{11C} is —C(O)NR₂. In some embodiments, R^{11C} is —C(O)N(R)OR. In some embodiments, R^{11C} is —OC(O)R. In some embodiments, R^{11C} is —OC(O)NR₂. In some embodiments, R^{11C} is —N(R)C(O)OR. In some embodiments, R^{11C} is —N(R)C(O)R. In some embodiments, R^{11C} is —N(R)C(O)NR₂. In some embodiments, R^{11C} is —N(R)C(NR)NR₂. In some embodiments, R^{11C} is —N(R)S(O)₂NR₂. In some embodiments, R^{11C} is —N(R)S(O)₂R. In some embodiments, R^{11C} is —P(O)R₂. In some embodiments, R^{11C} is —P(O)(R)OR. In some embodiments, R^{11C} is —B(OR)₂.

In some embodiments, each instance of R^{11C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{11C} is independently halogen, —CN, or —NO₂. In some embodiments, each instance of R^{11C} is independently —OR, —SR, or —NR₂. In some embodiments, each instance of R^{11C} is independently —S(O)₂R, —S(O)₂NR₂, —S(O)₂F, —S(O)R, —S(O)NR₂, or —S(O)(NR)R. In some embodiments, each instance of R^{11C} is independently —C(O)R, —C(O)OR, —C(O)NR₂, or —C(O)N(R)OR. In some embodiments, each instance of R^{11C} is independently —OC(O)R or —OC(O)NR₂. In some embodiments, each instance of R^{11C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{11C} is independently —P(O)R₂ or —P(O)(R)OR.

In some embodiments, each instance of R^{11C} is independently —OR, —OC(O)R, or —OC(O)NR₂. In some embodiments, each instance of R^{11C} is independently —SR, —S(O)₂R, —S(O)₂NR₂, —S(O)₂F, —S(O)R, —S(O)NR₂, or —S(O)(NR)R. In some embodiments, each instance of R^{11C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{11C} is independently —S(O)₂R, —S(O)₂NR₂, or —S(O)₂F. In some embodiments, each instance of R^{11C} is independently —S(O)R, —S(O)NR₂, or —S(O)(NR)R. In some embodiments, each instance of R^{11C} is independently —SR, —S(O)₂R, or —S(O)R. In some embodiments, each instance of R^{11C} is independently —S(O)₂NR₂, —S(O)NR₂, or —S(O)(NR)R. In some embodiments, each instance of R^{11C} is independently —S(O)₂NR₂ or —S(O)NR₂. In some embodiments, each instance of R^{11C} is independently —SR, —S(O)₂R, —S(O)₂NR₂, or —S(O)R.

In some embodiments, each instance of R^{11C} is independently —N(R)C(O)OR, —N(R)C(O)R, or —N(R)C(O)NR₂. In some embodiments, each instance of R^{11C} is independently —N(R)S(O)₂NR₂ or —N(R)S(O)₂R. In some embodiments, each instance of R^{11C} is independently —N(R)C(O)OR or —N(R)C(O)R. In some embodiments, each instance of R^{11C} is independently —N(R)C(O)NR₂ or —N(R)S(O)₂NR₂. In some embodiments, each instance of R^{11C} is independently —N(R)C(O)OR, —N(R)C(O)R, or —N(R)S(O)₂R.

In some embodiments, each instance of R^{11C} is independently —NR₂, —N(R)C(O)OR, —N(R)C(O)R, or —N(R)C(O)NR₂. In some embodiments, each instance of R^{11C} is independently —NR₂, —N(R)C(O)OR, or —N(R)C(O)R. In some embodiments, each instance of R^{11C} is independently —NR₂, —N(R)C(O)OR, —N(R)C(O)R, or —N(R)S(O)₂R.

In some embodiments, each instance of R^{11C} is independently an optionally substituted C₁₋₆ aliphatic. In some embodiments, each instance of R^{11C} is independently an optionally substituted phenyl. In some embodiments, each instance of R^{11C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{11C} is independently an optionally substituted 5-6 membered monocyclic heteroaryl ring having 1-4 heteroatoms independently selected from nitrogen, oxygen, and sulfur.

In some embodiments, each instance of R^{11C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{11C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{11C} is independently an optionally substituted C₁₋₆ aliphatic or an optionally substituted phenyl. In some embodiments, each instance of R^{11C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{11C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic. In some embodiments, R^{11C} is phenyl. In some embodiments, each instance of R^{11C} is independently a 3-7 membered saturated or partially unsaturated monocyclic heterocyclic ring having 1-2 heteroatoms independently selected from nitrogen, oxygen, and sulfur. In some embodiments, each instance of R^{11C} is independently a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{11C} is independently phenyl or a 5-6 membered monocyclic heteroaryl ring having 1-4 heteroatoms independently selected from nitrogen, oxygen, and sulfur.

In some embodiments, each instance of R^{11C} is independently a C₁₋₆ aliphatic or phenyl. In some embodiments, each instance of R^{11C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{11C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{11C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{11C} is independently halogen, —CN, —OH, —O-(optionally substituted C₁₋₃ aliphatic), or an optionally substituted C₁₋₃ aliphatic. In some embodiments, each instance of R^{11C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{11C} is independently fluorine, chlorine, —OH, —OCH₃, —OCF₃, —CH₃, —CHF₂, or —CF₃. In some embodiments, each instance of R^{11C} is independently fluorine or —OH.

In some embodiments, each instance of R^{11C} is independently oxo, deuterium, halogen, —CN, —OH, —O-(optionally substituted C₁₋₃ aliphatic), or an optionally substituted C₁₋₃ aliphatic. In some embodiments, each instance of R^{11C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{11C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{11C} is independently oxo, deuterium, fluorine, chlorine, —CN, —OH, —OCH₃, —OCF₃, —CH₃, —CHF₂, or —CF₃. In some embodiments, each instance of R^{11C} is independently oxo, deuterium, —CN, fluorine, or —OH. In some embodiments, each instance of R^{11C} is independently oxo, deuterium, —CN, —CH₃, or —CHF₂. In some embodiments, each instance of R^{11C} is independently deuterium, —CN, —CH₃, or —CHF₂.

In some embodiments, each instance of R^{11C} is independently oxo, halogen, —CN, —OH, —O-(optionally substituted C₁₋₃ aliphatic), or an optionally substituted C₁₋₃ aliphatic. In some embodiments, each instance of R^{11C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{11C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{11C} is independently oxo, fluorine, chlorine, —CN, —OH, —OCH₃, —OCF₃, —CH₃, —CHF₂, or —CF₃. In some embodiments, each instance of R^{11C} is independently oxo, —CN, fluorine, or —OH. In some embodiments, each instance of R^{11C} is independently oxo, —CN, —CH₃, or —CHF₂. In some embodiments, each instance of R^{11C} is independently —CN, —CH₃, or —CHF₂.

In some embodiments, each instance of R^{11C} is independently selected from the groups depicted in the compounds in Table 1.

As defined generally above, each instance of R^{22C} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{22C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{22C} is oxo. In some embodiments, R^{22C} is deuterium. In some embodiments, each instance of R^{22C} is independently halogen. In some embodiments, R^{22C} is —CN. In some embodiments, R^{22C} is —NO₂. In some embodiments, R^{22C} is —OR. In some embodiments, R^{22C} is —SR. In some embodiments, R^{22C} is —NR₂. In some embodiments, R^{22C} is —S(O)₂R. In some embodiments, R^{22C} is —S(O)₂NR₂. In some embodiments, R^{22C} is —S(O)₂F. In some embodiments, R^{22C} is —S(O)R. In some embodiments, R^{22C} is —S(O)NR₂. In some embodiments, R^{22C} is —S(O)(NR)R. In some embodiments, R^{22C} is —C(O)R. In some embodiments, R^{22C} is —C(O)OR. In some embodiments, R^{22C} is —C(O)NR₂. In some embodiments, R^{22C} is —C(O)N(R)OR. In some embodiments, R^{22C} is —OC(O)R. In some embodiments, R^{22C} is —OC(O)NR₂. In some embodiments, R^{22C} is —N(R)C(O)OR. In some embodiments, R^{22C} is —N(R)C(O)R. In some embodiments, R^{22C} is —N(R)C(O)NR₂. In some embodiments, R^{22C} is —N(R)C(NR)NR₂. In some embodiments, R^{22C} is —N(R)S(O)₂NR₂. In some embodiments, R^{22C} is —N(R)S(O)₂R. In some embodiments, R^{22C} is —P(O)R₂. In some embodiments, R^{22C} is —P(O)(R)OR. In some embodiments, R^{22C} is —B(OR)₂.

In some embodiments, each instance of R^{22C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{22C} is independently halogen, —CN, or —NO₂. In some embodiments, each instance of R^{22C} is independently —OR, —SR, or —NR₂. In some embodiments, each instance of R^{22C} is independently —S(O)₂R, —S(O)₂NR₂, —S(O)₂F, —S(O)R, —S(O)NR₂, or —S(O)(NR)R. In some embodiments, each instance of R^{22C} is independently —C(O)R, —C(O)OR, —C(O)NR₂, or —C(O)N(R)OR. In some embodiments, each instance of R^{22C} is independently —OC(O)R or —OC(O)NR₂. In some embodiments, each instance of R^{22C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{22C} is independently —P(O)R₂ or —P(O)(R)OR.

In some embodiments, each instance of R^{22C} is independently —OR, —OC(O)R, or —OC(O)NR₂. In some embodiments, each instance of R^{22C} is independently —SR, —S(O)₂R, —S(O)₂NR₂, —S(O)₂F, —S(O)R, —S(O)NR₂, or —S(O)(NR)R. In some embodiments, each instance of R^{22C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{22C} is independently —S(O)₂R, —S(O)₂NR₂, or —S(O)₂F. In some embodiments, each instance of R^{22C} is independently —S(O)R, —S(O)NR₂, or —S(O)(NR)R. In some embodiments, each instance of R^{22C} is independently —SR, —S(O)₂R, or —S(O)R. In some embodiments, each instance of R^{22C} is independently —S(O)₂NR₂, —S(O)NR₂, or —S(O)(NR)R. In some embodiments, each instance of R^{22C} is independently —S(O)₂NR₂ or —S(O)NR₂. In some embodiments, each instance of R^{22C} is independently —SR, —S(O)₂R, —S(O)₂NR₂, or —S(O)R.

In some embodiments, each instance of R^{22C} is independently —N(R)C(O)OR, —N(R)C(O)R, or —N(R)C(O)NR₂. In some embodiments, each instance of R^{22C} is independently —N(R)S(O)₂NR₂ or —N(R)S(O)₂R. In some embodiments, each instance of R^{22C} is independently —N(R)C(O)OR or —N(R)C(O)R. In some embodiments, each instance of R^{22C} is independently —N(R)C(O)NR₂ or —N(R)S(O)₂NR₂. In some embodiments, each instance of R^{22C} is independently —N(R)C(O)OR, —N(R)C(O)R, or —N(R)S(O)₂R.

In some embodiments, each instance of R^{22C} is independently —NR₂, —N(R)C(O)OR, —N(R)C(O)R, or —N(R)C(O)NR₂. In some embodiments, each instance of R^{22C} is independently —NR₂, —N(R)C(O)OR, or —N(R)C(O)R. In some embodiments, each instance of R^{22C} is independently —NR₂, —N(R)C(O)OR, —N(R)C(O)R, or —N(R)S(O)₂R.

In some embodiments, each instance of R^{22C} is independently an optionally substituted C₁₋₆ aliphatic. In some embodiments, each instance of R^{22C} is independently an optionally substituted phenyl. In some embodiments, each instance of R^{22C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{22C} is independently an optionally substituted 5-6 membered monocyclic heteroaryl ring having 1-4 heteroatoms independently selected from nitrogen, oxygen, and sulfur.

In some embodiments, each instance of R^{22C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{22C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{22C} is independently an optionally substituted C₁₋₆ aliphatic or an optionally substituted phenyl. In some embodiments, each instance of R^{22C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{22C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic. In some embodiments, R^{22C} is phenyl. In some embodiments, each instance of R^{22C} is independently a 3-7 membered saturated or partially unsaturated monocyclic heterocyclic ring having 1-2 heteroatoms independently selected from nitrogen, oxygen, and sulfur. In some embodiments, each instance of R^{22C} is independently a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{22C} is independently phenyl or a 5-6 membered monocyclic heteroaryl ring having 1-4 heteroatoms independently selected from nitrogen, oxygen, and sulfur.

In some embodiments, each instance of R^{22C} is independently a C₁₋₆ aliphatic or phenyl. In some embodiments, each instance of R^{22C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{22C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{22C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{22C} is independently halogen, —CN, —OH, —O-(optionally substituted C₁₋₃ aliphatic), or an optionally substituted C₁₋₃ aliphatic. In some embodiments, each instance of R^{22C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently fluorine, chlorine, —OH, —OCH₃, —OCF₃, —CH₃, —CHF₂, or —CF₃. In some embodiments, each instance of R^{22C} is independently fluorine or —OH.

In some embodiments, each instance of R^{22C} is independently oxo, deuterium, halogen, —CN, —OH, —O-(optionally substituted C₁₋₃ aliphatic), or an optionally substituted C₁₋₃ aliphatic. In some embodiments, each instance of R^{22C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{22C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently oxo, deuterium, fluorine, chlorine, —CN, —OH, —OCH₃, —OCF₃, —CH₃, —CHF₂, or —CF₃. In some embodiments, each instance of R^{22C} is independently oxo, deuterium, —CN, fluorine, or —OH. In some embodiments, each instance of R^{22C} is independently oxo, deuterium, —CN, —CH₃, or —CHF₂. In some embodiments, each instance of R^{22C} is independently deuterium, —CN, —CH₃, or —CHF₂.

In some embodiments, each instance of R^{22C} is independently oxo, halogen, —CN, —OH, —O-(optionally substituted C₁₋₃ aliphatic), or an optionally substituted C₁₋₃ aliphatic. In some embodiments, each instance of R^{22C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{22C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{22C} is independently oxo, fluorine, chlorine, —CN, —OH, —OCH₃, —OCF₃, —CH₃, —CHF₂, or —CF₃. In some embodiments, each instance of R^{22C} is independently oxo, —CN, fluorine, or —OH. In some embodiments, each instance of R^{22C} is independently oxo, —CN, —CH₃, or —CHF₂. In some embodiments, each instance of R^{22C} is independently —CN, —CH₃, or —CHF₂.

In some embodiments, each instance of R^{22C} is independently selected from the groups depicted in the compounds in Table 1.

As defined generally above, each instance of R^{T}c is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{T1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{T1C} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{T1C} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{T1C} is oxo. In some embodiments, R^{T1C} is deuterium. In some embodiments, each instance of R^{T1C} is independently halogen. In some embodiments, R^{T1C} is —CN. In some embodiments, R^{T1C} is —NO₂. In some embodiments, R^{T1C} is —OR. In some embodiments, R^{T1C} is —SR. In some embodiments, R^{T1C} is —NR₂. In some embodiments, R^{T1C} is —S(O)₂R. In some embodiments, R^{T1C} is —S(O)₂NR₂. In some embodiments, R^{T1C} is —S(O)₂F. In some embodiments, R^{T1C} is —S(O)R. In some embodiments, R^{T1C} is —S(O)NR₂. In some embodiments, R^{T1C} is —S(O)(NR)R. In some embodiments, R^{T1C} is —C(O)R. In some embodiments, R^{T1C} is —C(O)OR. In some embodiments, R^{T1C} is —C(O)NR₂. In some embodiments, R^{T1C} is —C(O)N(R)OR. In some embodiments, R^{T1C} is —OC(O)R. In some embodiments, R^{T1C} is —OC(O)NR₂. In some embodiments, R^{T1C} is —N(R)C(O)OR. In some embodiments, R^{T1C} is —N(R)C(O)R. In some embodiments, R^{T1C} is —N(R)C(O)NR₂. In some embodiments, R^{T1C} is —N(R)C(NR)NR₂. In some embodiments, R^{T1C} is —N(R)S(O)₂NR₂. In some embodiments, R^{T1C} is —N(R)S(O)₂R. In some embodiments, R^{T1C} is —P(O)R₂. In some embodiments, R^{T1C} is —P(O)(R)OR. In some embodiments, R^{T1C} is —B(OR)₂.

In some embodiments, each instance of R^{T1C} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{T1C} is independently halogen, —CN, or —NO₂. In some embodiments, each instance of R^{T1C} is independently —OR, —SR, or —NR₂. In some embodiments, each instance of R^{T1C} is independently —S(O)₂R, —S(O)₂NR₂, —S(O)₂F, —S(O)R, —S(O)NR₂, or —S(O)(NR)R. In some embodiments, each instance of R^{T1C} is independently —C(O)R, —C(O)OR, —C(O)NR₂, or —C(O)N(R)OR. In some embodiments, each instance of R^{T1C} is independently —OC(O)R or —OC(O)NR₂. In some embodiments, each instance of R^{T1C} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{T1C} is independently —P(O)R₂ or —P(O)(R)OR.

In some embodiments, each instance of R^{T1C} is independently —OR, —OC(O)R, or —OC(O)NR₂. In some embodiments, each instance of R^{T1C} is independently —SR, —S(O)₂R, —S(O)₂NR₂, —S(O)₂F, —S(O)R, —S(O)NR₂, or —S(O)(NR)R. In some embodiments, each instance of R^{T1C} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{T1C} is independently —S(O)₂R, —S(O)₂NR₂, or —S(O)₂F. In some embodiments, each instance of R^{T1C} is independently —S(O)R, —S(O)NR₂, or —S(O)(NR)R. In some embodiments, each instance of R^{T1C} is independently —SR, —S(O)₂R, or —S(O)R. In some embodiments, each instance of R^{T1C} is independently —S(O)₂NR₂, —S(O)NR₂, or —S(O)(NR)R. In some embodiments, each instance of R^{T1C} is independently —S(O)₂NR₂ or —S(O)NR₂. In some embodiments, each instance of R^{T1C} is independently —SR, —S(O)₂R, —S(O)₂NR₂, or —S(O)R.

In some embodiments, each instance of R^{T1C} is independently —N(R)C(O)OR, —N(R)C(O)R, or —N(R)C(O)NR₂. In some embodiments, each instance of R^{T1C} is independently —N(R)S(O)₂NR₂ or —N(R)S(O)₂R. In some embodiments, each instance of R^{T1C} is independently —N(R)C(O)OR or —N(R)C(O)R. In some embodiments, each instance of R^{T1C} is independently —N(R)C(O)NR₂ or —N(R)S(O)₂NR₂. In some embodiments, each instance of R^{T1C} is independently —N(R)C(O)OR, —N(R)C(O)R, or —N(R)S(O)₂R.

In some embodiments, each instance of R^{T1C} is independently —NR₂, —N(R)C(O)OR, —N(R)C(O)R, or —N(R)C(O)NR₂. In some embodiments, each instance of R^{T1C} is independently —NR₂, —N(R)C(O)OR, or —N(R)C(O)R. In some embodiments, each instance of R^{T1C} is independently —NR₂, —N(R)C(O)OR, —N(R)C(O)R, or —N(R)S(O)₂R.

In some embodiments, each instance of R^{T1C} is independently an optionally substituted C₁₋₆ aliphatic. In some embodiments, each instance of R^{T1C} is independently an optionally substituted phenyl. In some embodiments, each instance of R^{T1C} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{T1C} is independently an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{T1C} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted C₁₋₆ aliphatic or an optionally substituted phenyl. In some embodiments, each instance of R^{T1C} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{T1C} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{T1C} is independently a C₁₋₆ aliphatic. In some embodiments, R^{T1C} is phenyl. In some embodiments, each instance of R^{T1C} is independently a 3-7 membered saturated or partially unsaturated monocyclic heterocyclic ring having 1-2 heteroatoms independently selected from nitrogen, oxygen, and sulfur. In some embodiments, each instance of R^{T1C} is independently a 5-6 membered monocyclic heteroaryl ring having 1-4 heteroatoms independently selected from nitrogen, oxygen, and sulfur.

In some embodiments, each instance of R^{T1C} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{T1C} is independently phenyl or a 5-6 membered monocyclic heteroaryl ring having 1-4 heteroatoms independently selected from nitrogen, oxygen, and sulfur.

In some embodiments, each instance of R^{T1C} is independently a C₁₋₆ aliphatic or phenyl. In some embodiments, each instance of R^{T1C} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{T1C} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{T1C} is independently halogen, —CN, —OH, —O-(optionally substituted C₁₋₃ aliphatic), or an optionally substituted C₁₋₃ aliphatic. In some embodiments, each instance of R^{T1C} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently fluorine, chlorine, —OH, —OCH₃, —OCF₃, —CH₃, —CHF₂, or —CF₃. In some embodiments, each instance of R^{T1C} is independently fluorine or —OH.

In some embodiments, each instance of R^{T1C} is independently oxo, deuterium, halogen, —CN, —OH, —O-(optionally substituted C₁₋₃ aliphatic), or an optionally substituted C₁₋₃ aliphatic. In some embodiments, each instance of R^{T1C} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{T1C} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently oxo, deuterium, fluorine, chlorine, —CN, —OH, —OCH₃, —OCF₃, —CH₃, —CHF₂, or —CF₃. In some embodiments, each instance of R^{T1C} is independently oxo, deuterium, —CN, fluorine, or —OH. In some embodiments, each instance of R^{T1C} is independently oxo, deuterium, —CN, —CH₃, or —CHF₂. In some embodiments, each instance of R^{T1C} is independently deuterium, —CN, —CH₃, or —CHF₂.

In some embodiments, each instance of R^{T1C} is independently oxo, halogen, —CN, —OH, —O-(optionally substituted C₁₋₃ aliphatic), or an optionally substituted C₁₋₃ aliphatic. In some embodiments, each instance of R^{T1C} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{T1C} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{T1C} is independently oxo, fluorine, chlorine, —CN, —OH, —OCH₃, —OCF₃, —CH₃, —CHF₂, or —CF₃. In some embodiments, each instance of R^{T1C} is independently oxo, —CN, fluorine, or —OH. In some embodiments, each instance of R^{T1C} is independently oxo, —CN, —CH₃, or —CHF₂. In some embodiments, each instance of R^{T1C} is independently —CN, —CH₃, or —CHF₂.

In some embodiments, each instance of R^{T1C} is independently selected from the groups depicted in the compounds in Table 1.

As defined generally above, each instance of R^{TLC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{TLC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{TLC} is oxo. In some embodiments, R^{TLC} is deuterium. In some embodiments, each instance of R^{TLC} is independently halogen. In some embodiments, R^{TLC} is —CN. In some embodiments, R^{TLC} is —NO₂. In some embodiments, R^{TLC} is —OR. In some embodiments, R^{TLC} is —SR. In some embodiments, R^{TLC} is —NR₂. In some embodiments, R^{TLC} is —S(O)₂R. In some embodiments, R^{TLC} is —S(O)₂NR₂. In some embodiments, R^{TLC} is —S(O)₂F. In some embodiments, R^{TLC} is —S(O)R. In some embodiments, R^{TLC} is —S(O)NR₂. In some embodiments, R^{TLC} is —S(O)(NR)R. In some embodiments, R^{TLC} is —C(O)R. In some embodiments, R^{TLC} is —C(O)OR. In some embodiments, R^{TLC} is —C(O)NR₂. In some embodiments, R^{TLC} is —C(O)N(R)OR. In some embodiments, R^{TLC} is —OC(O)R. In some embodiments, R^{TLC} is —OC(O)NR₂. In some embodiments, R^{TLC} is —N(R)C(O)OR. In some embodiments, R^{TLC} is —N(R)C(O)R. In some embodiments, R^{TLC} is —N(R)C(O)NR₂. In some embodiments, R^{TLC} is —N(R)C(NR)NR₂. In some embodiments, R^{TLC} is —N(R)S(O)₂NR₂. In some embodiments, R^{TLC} is —N(R)S(O)₂R. In some embodiments, R^{TLC} is —P(O)R₂. In some embodiments, R^{TLC} is —P(O)(R)OR. In some embodiments, R^{TLC} is —B(OR)₂.

In some embodiments, each instance of R^{TLC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{TLC} is independently halogen, —CN, or —NO₂. In some embodiments, each instance of R^{TLC} is independently —OR, —SR, or —NR₂. In some embodiments, each instance of R^{TLC} is independently —S(O)₂R, —S(O)₂NR₂, —S(O)₂F, —S(O)R, —S(O)NR₂, or —S(O)(NR)R. In some embodiments, each instance of R^{TLC} is independently —C(O)R, —C(O)OR, —C(O)NR₂, or —C(O)N(R)OR. In some embodiments, each instance of R^{TLC} is independently —OC(O)R or —OC(O)NR₂. In some embodiments, each instance of R^{TLC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{TLC} is independently —P(O)R₂ or —P(O)(R)OR.

In some embodiments, each instance of R^{TLC} is independently —OR, —OC(O)R, or —OC(O)NR₂. In some embodiments, each instance of R^{TLC} is independently —SR, —S(O)₂R, —S(O)₂NR₂, —S(O)₂F, —S(O)R, —S(O)NR₂, or —S(O)(NR)R. In some embodiments, each instance of R^{TLC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{TLC} is independently —S(O)₂R, —S(O)₂NR₂, or —S(O)₂F. In some embodiments, each instance of R^{TLC} is
independently —S(O)R, —S(O)NR₂, or —S(O)(NR)R. In some embodiments, each instance of R^{TLC} is independently —SR, —S(O)₂R, or —S(O)R. In some embodiments, each instance of R^{TLC} is independently —S(O)₂NR₂,
—S(O)NR₂, or —S(O)(NR)R. In some embodiments, each instance of R^{TLC} is independently —S(O)₂NR₂ or —S(O)NR₂. In some embodiments, each instance of R^{TLC} is independently —SR, —S(O)₂R, —S(O)₂NR₂, or —S(O)R.

In some embodiments, each instance of R^{TLC} is independently —N(R)C(O)OR, —N(R)C(O)R, or —N(R)C(O)NR₂. In some embodiments, each instance of R^{TLC} is independently —N(R)S(O)₂NR₂ or —N(R)S(O)₂R. In some embodiments, each instance of R^{TLC} is independently —N(R)C(O)OR or —N(R)C(O)R. In some embodiments, each instance of R^{TLC} is independently —N(R)C(O)NR₂ or —N(R)S(O)₂NR₂. In some embodiments, each instance of R^{TLC} is independently —N(R)C(O)OR, —N(R)C(O)R, or —N(R)S(O)₂R.

In some embodiments, each instance of R^{TLC} is independently —NR₂, —N(R)C(O)OR, —N(R)C(O)R, or —N(R)C(O)NR₂. In some embodiments, each instance of R^{TLC} is independently —NR₂, —N(R)C(O)OR, or —N(R)C(O)R. In some embodiments, each instance of R^{TLC} is independently —NR₂, —N(R)C(O)OR, —N(R)C(O)R, or —N(R)S(O)₂R.

In some embodiments, each instance of R^{TLC} is independently an optionally substituted C₁₋₆ aliphatic. In some embodiments, each instance of R^{TLC} is independently an optionally substituted phenyl. In some embodiments, each instance of R^{TLC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{TLC} is independently an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{TLC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted C₁₋₆ aliphatic or an optionally substituted phenyl. In some embodiments, each instance of R^{TLC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{TLC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic. In some embodiments, R^{TLC} is phenyl. In some embodiments, each instance of R^{TLC} is independently a 3-7 membered saturated or partially unsaturated monocyclic heterocyclic ring having 1-2 heteroatoms independently selected from nitrogen, oxygen, and sulfur. In some embodiments, each instance of R^{TLC} is independently a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{TLC} is independently phenyl or a 5-6 membered monocyclic heteroaryl ring having 1-4 heteroatoms independently selected from nitrogen, oxygen, and sulfur.

In some embodiments, each instance of R^{TLC} is independently a C₁₋₆ aliphatic or phenyl. In some embodiments, each instance of R^{TLC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TLC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{TLC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or optionally substituted C₁₋₆ aliphatic.

In some embodiments, each instance of R^{TLC} is independently halogen, —CN, —OH, —O-(optionally substituted C₁₋₃ aliphatic), or an optionally substituted C₁₋₃ aliphatic. In some embodiments, each instance of R^{TLC} is independently halogen, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently fluorine, chlorine, —OH, —OCH₃, —OCF₃, —CH₃, —CHF₂, or —CF₃. In some embodiments, each instance of R^{TLC} is independently fluorine or —OH.

In some embodiments, each instance of R^{TLC} is independently oxo, deuterium, halogen, —CN, —OH, —O-(optionally substituted C₁₋₃ aliphatic), or an optionally substituted C₁₋₃ aliphatic. In some embodiments, each instance of R^{TLC} is independently oxo, deuterium, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TLC} is independently oxo, deuterium, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently oxo, deuterium, fluorine, chlorine, —CN, —OH, —OCH₃, —OCF₃, —CH₃, —CHF₂, or —CF₃. In some embodiments, each instance of R^{TLC} is independently oxo, deuterium, —CN, fluorine, or —OH. In some embodiments, each instance of R^{TLC} is independently oxo, deuterium, —CN, —CH₃, or —CHF₂. In some embodiments, each instance of R^{TLC} is independently deuterium, —CN, —CH₃, or —CHF₂.

In some embodiments, each instance of R^{TLC} is independently oxo, halogen, —CN, —OH, —O-(optionally substituted C₁₋₃ aliphatic), or an optionally substituted C₁₋₃ aliphatic. In some embodiments, each instance of R^{TLC} is independently oxo, halogen, —CN, —OH, —O—(C₁₋₃ aliphatic), or C₁₋₃ aliphatic, wherein each C₁₋₃ aliphatic is optionally substituted with one or more halogen atoms. In some embodiments, each instance of R^{TLC} is independently oxo, halogen, —CN, —OH, —O—(C₁₋₃ aliphatic), or C₁₋₃ aliphatic, wherein each C₁₋₃ aliphatic is optionally substituted with 1-3 halogen. In some embodiments, each instance of R^{TLC} is independently oxo, fluorine, chlorine, —CN, —OH, —OCH₃, —OCF₃, —CH₃, —CHF₂, or —CF₃. In some embodiments, each instance of R^{TLC} is independently oxo, —CN, fluorine, or —OH. In some embodiments, each instance of R^{TLC} is independently oxo, —CN, —CH₃, or —CHF₂. In some embodiments, each instance of R^{TLC} is independently —CN, —CH₃, or —CHF₂.

In some embodiments, each instance of R^{TLC} is independently selected from the groups depicted in the compounds in Table 1.

As defined generally above, each instance of R^{LC} is independently oxo, deuterium, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, —B(OR)₂, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently oxo, halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂. In some embodiments, each instance of R^{LC} is independently an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{LC} is oxo. In some embodiments, R^{LC} is deuterium. In some embodiments, each instance of R^{LC} is independently halogen. In some embodiments, R^{LC} is —CN. In some embodiments, R^{LC} is —NO₂. In some embodiments, R^{LC} is —OR. In some embodiments, R^{LC} is —SR. In some embodiments, R^{LC} is —NR₂. In some embodiments, R^{LC} is —S(O)₂R. In some embodiments, R^{LC} is —S(O)₂NR₂. In some embodiments, R^{LC} is —S(O)₂F. In some embodiments, R^{LC} is —S(O)R. In some embodiments, R^{LC} is —S(O)NR₂. In some embodiments, R^{LC} is —S(O)(NR)R. In some embodiments, R^{LC} is —C(O)R. In some embodiments, R^{LC} is —C(O)OR. In some embodiments, R^{LC} is —C(O)NR₂. In some embodiments, R^{LC} is —C(O)N(R)OR. In some embodiments, R^{LC} is —OC(O)R. In some embodiments, R^{LC} is —OC(O)NR₂. In some embodiments, R^{LC} is —N(R)C(O)OR. In some embodiments, R^{LC} is —N(R)C(O)R. In some embodiments, R^{LC} is —N(R)C(O)NR₂. In some embodiments, R^{LC} is —N(R)C(NR)NR₂. In some embodiments, R^{LC} is —N(R)S(O)₂NR₂. In some embodiments, R^{LC} is —N(R)S(O)₂R. In some embodiments, R^{LC} is —P(O)R₂. In some embodiments, R^{LC} is —P(O)(R)OR. In some embodiments, R^{LC} is —B(OR)₂.

In some embodiments, each instance of R^{LC} is independently halogen, —CN, —NO₂, —OR, —SR, —NR₂, —S(O)₂R, —S(O)₂NR₂, —S(O)₂F, —S(O)R, —S(O)NR₂, —S(O)(NR)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, —P(O)R₂, —P(O)(R)OR, or —B(OR)₂.

In some embodiments, each instance of R^{LC} is independently halogen, —CN, or —NO₂. In some embodiments, each instance of R^{LC} is independently —OR, —SR, or —NR₂. In some embodiments, each instance of R^{LC} is independently —S(O)₂R, —S(O)₂NR₂, —S(O)₂F, —S(O)R, —S(O)NR₂, or —S(O)(NR)R. In some embodiments, each instance of R^{LC} is independently —C(O)R, —C(O)OR, —C(O)NR₂, or —C(O)N(R)OR. In some embodiments, each instance of R^{LC} is independently —OC(O)R or —OC(O)NR₂. In some embodiments, each instance of R^{LC} is independently —N(R)C(O)OR, —N(R)C(O)R, —N(R)C(O)NR₂, —N(R)C(NR)NR₂, —N(R)S(O)₂NR₂, or —N(R)S(O)₂R. In some embodiments, each instance of R^{LC} is independently —P(O)R₂ or —P(O)(R)OR.

In some embodiments, each instance of R^{LC} is independently —OR, —OC(O)R, or —OC(O)NR₂. In some embodiments, each instance of R^{LC} is independently —SR, —S(O)₂R, —S(O)₂NR₂, —S(O)₂F, —S(O)R, —S(O)NR₂, or —S(O)(NR)R. In some embodiments, each instance of R^{LC} is independently —NR₂, —N(R)C(O)OR, —N(R)C(O)R, —N(R)C(O)NR₂, —N(R)C(NR)NR₂, —N(R)S(O)₂NR₂, or —N(R)S(O)₂R.

In some embodiments, each instance of R^{LC} is independently —S(O)₂R, —S(O)₂NR₂, or —S(O)₂F. In some embodiments, each instance of R^{LC} is independently —S(O)R, —S(O)NR₂, or —S(O)(NR)R. In some embodiments, each instance of R^{LC} is independently —SR, —S(O)₂R, or —S(O)R. In some embodiments, each instance of R^{LC} is independently —S(O)₂NR₂, —S(O)NR₂, or —S(O)(NR)R. In some embodiments, each instance of R^{LC} is independently —S(O)₂NR₂ or —S(O)NR₂. In some embodiments, each instance of R^{LC} is independently —SR, —S(O)₂R, —S(O)₂NR₂, or —S(O)R.

In some embodiments, each instance of R^{LC} is independently —N(R)C(O)OR, —N(R)C(O)R, or —N(R)C(O)NR₂. In some embodiments, each instance of R^{LC} is independently —N(R)S(O)₂NR₂ or —N(R)S(O)₂R. In some embodiments, each instance of R^{LC} is independently —N(R)C(O)OR or —N(R)C(O)R. In some embodiments, each instance of R^{LC} is independently —N(R)C(O)NR₂ or —N(R)S(O)₂NR₂. In some embodiments, each instance of R^{LC} is independently —N(R)C(O)OR, —N(R)C(O)R, or —N(R)S(O)₂R.

In some embodiments, each instance of R^{LC} is independently —NR₂, —N(R)C(O)OR, —N(R)C(O)R, or —N(R)C(O)NR₂. In some embodiments, each instance of R^{LC} is independently —NR₂, —N(R)C(O)OR, or —N(R)C(O)R. In some embodiments, each instance of R^{LC} is independently —NR₂, —N(R)C(O)OR, —N(R)C(O)R, or —N(R)S(O)₂R.

In some embodiments, each instance of R^{LC} is independently an optionally substituted C₁₋₆ aliphatic. In some embodiments, each instance of R^{LC} is independently an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, each instance of R^{LC} is independently an optionally substituted phenyl or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted C₁₋₆ aliphatic or an optionally substituted phenyl. In some embodiments, each instance of R^{LC} is independently an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, each instance of R^{LC} is independently an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic. In some embodiments, R^{LC} is phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur. In some embodiments, each instance of R^{LC} is independently a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or a 3-7 membered saturated or partially unsaturated monocyclic heterocyclic ring having 1-2 heteroatoms independently selected from nitrogen, oxygen, and sulfur. In some embodiments, each instance of R^{LC} is independently phenyl or a 5-6 membered monocyclic heteroaryl ring having 1-4 heteroatoms independently selected from nitrogen, oxygen, and sulfur.

In some embodiments, each instance of R^{LC} is independently a C₁₋₆ aliphatic or phenyl. In some embodiments, each instance of R^{LC} is independently a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, each instance of R^{LC} is independently selected from the groups depicted in the compounds in Table 1.

As defined generally above, each instance of R is independently hydrogen, or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur; or two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen or an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is an optionally substituted group selected from C₁₋₆ aliphatic, phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen, C₁₋₆ aliphatic, phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is an optionally substituted C₁₋₆ aliphatic. In some embodiments, R is an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted 3-7 membered saturated or partially unsaturated monocyclic heterocyclic ring having 1-2 heteroatoms independently selected from nitrogen, oxygen, and sulfur. In some embodiments, R is an optionally substituted phenyl or an optionally substituted 5-6 membered monocyclic heteroaryl ring having 1-4 heteroatoms independently selected from nitrogen, oxygen, and sulfur.

In some embodiments, R is an optionally substituted C₁₋₆ aliphatic or an optionally substituted phenyl. In some embodiments, R is an optionally substituted 3-7 membered saturated or partially unsaturated monocyclic heterocyclic ring having 1-2 heteroatoms independently selected from nitrogen, oxygen, and sulfur, or an optionally substituted 5-6 membered monocyclic heteroaryl ring having 1-4 heteroatoms independently selected from nitrogen, oxygen, and sulfur.

In some embodiments, R is an optionally substituted group selected from phenyl, a 3-7 membered saturated or partially unsaturated monocyclic heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is a C₁₋₆ aliphatic. In some embodiments, R is phenyl. In some embodiments, R is a 3-7 membered saturated or partially unsaturated monocyclic heterocyclic ring having 1-2 heteroatoms independently selected from nitrogen, oxygen, and sulfur. In some embodiments, R is a 5-6 membered monocyclic heteroaryl ring having 1-4 heteroatoms independently selected from nitrogen, oxygen, and sulfur.

In some embodiments, R is a C₁₋₆ aliphatic or a 3-7 membered saturated or partially unsaturated monocyclic heterocyclic ring having 1-2 heteroatoms independently selected from nitrogen, oxygen, and sulfur. In some embodiments, R is phenyl or a 5-6 membered monocyclic heteroaryl ring having 1-4 heteroatoms independently selected from nitrogen, oxygen, and sulfur.

In some embodiments, R is a C₁₋₆ aliphatic or phenyl. In some embodiments, R is a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, R is phenyl, a 3-7 membered saturated or partially unsaturated monocyclic heterocyclic ring having 1-2 heteroatoms independently selected from nitrogen, oxygen, and sulfur, or a 5-6 membered monocyclic heteroaryl ring having 1-4 heteroatoms independently selected from nitrogen, oxygen, and sulfur.

In some embodiments, two R groups on the same nitrogen are taken together with their intervening atoms to form a 4-7 membered saturated, partially unsaturated, or heteroaryl ring having 1-3 heteroatoms, in addition to the nitrogen, independently selected from nitrogen, oxygen, and sulfur. In some embodiments, two R groups on the same nitrogen are taken together with their intervening atoms to form a 4-7 membered saturated, partially unsaturated, or heteroaryl ring having no additional heteroatoms other than said nitrogen.

In some embodiments, two R groups on the same nitrogen are taken together with their intervening atoms to form a 4-7 membered saturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 0-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 0-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered partially unsaturated ring having 1-3 heteroatoms, in addition to the nitrogen, independently selected from nitrogen, oxygen, and sulfur. In some embodiments, two R groups on the same nitrogen are taken together with their intervening atoms to form a 4-7 membered heteroaryl ring having 1-3 heteroatoms, in addition to the nitrogen, independently selected from nitrogen, oxygen, and sulfur.

In some embodiments, two R groups on the same nitrogen are taken together with their intervening atoms to form a 4-7 membered saturated ring having no additional heteroatoms other than said nitrogen. In some embodiments, two R groups on the same nitrogen are taken together with their intervening atoms to form a 4-7 membered partially unsaturated ring having no additional heteroatoms other than said nitrogen. In some embodiments, two R groups on the same nitrogen are taken together with their intervening atoms to form a 4-7 membered heteroaryl ring having no additional heteroatoms other than said nitrogen.

In some embodiments, R is selected from the groups depicted in the compounds in Table 1.

As defined generally above, n is 0, 1, 2, 3, 4, or 5. In some embodiments, n is 0. In some embodiments, n is 1. In some embodiments, n is 2. In some embodiments, n is 3. In some embodiments, n is 4. In some embodiments, n is 5. In some embodiments, n is 0 or 1. In some embodiments, n is 0, 1, or 2. In some embodiments, n is 0, 1, 2, or 3. In some embodiments, n is 0, 1, 2, 3, or 4. In some embodiments, n is 1 or 2. In some embodiments, n is 1, 2, or 3. In some embodiments, n is 1, 2, 3, or 4. In some embodiments, n is 1, 2, 3, 4, or 5. In some embodiments, n is 2 or 3. In some embodiments, n is 2, 3, or 4. In some embodiments, n is 2, 3, 4, or 5. In some embodiments, n is 3 or 4. In some embodiments, n is 3, 4, or 5. In some embodiments, n is 4 or 5. In some embodiments, n is selected from the values represented in the compounds in Table 1.

As defined generally above, m is 0, 1, 2, 3, 4, or 5. In some embodiments, m is 0. In some embodiments, m is 1. In some embodiments, m is 2. In some embodiments, m is 3. In some embodiments, m is 4. In some embodiments, m is 5. In some embodiments, m is 0 or 1. In some embodiments, m is 0, 1, or 2. In some embodiments, m is 0, 1, 2, or 3. In some embodiments, m is 0, 1, 2, 3, or 4. In some embodiments, m is 1 or 2. In some embodiments, m is 1, 2, or 3. In some embodiments, m is 1, 2, 3, or 4. In some embodiments, m is 1, 2, 3, 4, or 5. In some embodiments, m is 2 or 3. In some embodiments, m is 2, 3, or 4. In some embodiments, m is 2, 3, 4, or 5. In some embodiments, m is 3 or 4. In some embodiments, m is 3, 4, or 5. In some embodiments, m is 4 or 5. In some embodiments, m is selected from the values represented in the compounds in Table 1.

As defined generally above, q is 0, 1, 2, 3, 4, or 5. In some embodiments, q is 0. In some embodiments, q is 1. In some embodiments, q is 2. In some embodiments, q is 3. In some embodiments, q is 4. In some embodiments, q is 5. In some embodiments, q is 0 or 1. In some embodiments, q is 0, 1, or 2. In some embodiments, q is 0, 1, 2, or 3. In some embodiments, q is 0, 1, 2, 3, or 4. In some embodiments, q is 1 or 2. In some embodiments, q is 1, 2, or 3. In some embodiments, q is 1, 2, 3, or 4. In some embodiments, q is 1, 2, 3, 4, or 5. In some embodiments, q is 2 or 3. In some embodiments, q is 2, 3, or 4. In some embodiments, q is 2, 3, 4, or 5. In some embodiments, q is 3 or 4. In some embodiments, q is 3, 4, or 5. In some embodiments, q is 4 or 5. In some embodiments, q is selected from the values represented in the compounds in Table 1.

As defined generally above, p¹ is 0, 1, 2, 3, 4, or 5. In some embodiments, p¹ is 0. In some embodiments, p¹ is 1. In some embodiments, p¹ is 2. In some embodiments, p¹ is 3. In some embodiments, p¹ is 4. In some embodiments, p¹ is 5. In some embodiments, p¹ is 0 or 1. In some embodiments, p¹ is 0, 1, or 2. In some embodiments, p¹ is 0, 1, 2, or 3. In some embodiments, p¹ is 0, 1, 2, 3, or 4. In some embodiments, p¹ is 1 or 2. In some embodiments, p¹ is 1, 2, or 3. In some embodiments, p¹ is 1, 2, 3, or 4. In some embodiments, p¹ is 1, 2, 3, 4, or 5. In some embodiments, p¹ is 2 or 3. In some embodiments, p¹ is 2, 3, or 4. In some embodiments, p¹ is 2, 3, 4, or 5. In some embodiments, p¹ is 3 or 4. In some embodiments, p¹ is 3, 4, or 5. In some embodiments, p¹ is selected from the values represented in the compounds in Table 1.

As defined generally above, p² is 0, 1, 2, 3, 4, or 5. In some embodiments, p² is 0. In some embodiments, p² is 1. In some embodiments, p² is 2. In some embodiments, p² is 3. In some embodiments, p² is 4. In some embodiments, p² is 5. In some embodiments, p² is 0 or 1. In some embodiments, p² is 0, 1, or 2. In some embodiments, p² is 0, 1, 2, or 3. In some embodiments, p² is 0, 1, 2, 3, or 4. In some embodiments, p² is 1 or 2. In some embodiments, p² is 1, 2, or 3. In some embodiments, p² is 1, 2, 3, or 4. In some embodiments, p² is 1, 2, 3, 4, or 5. In some embodiments, p² is 2 or 3. In some embodiments, p² is 2, 3, or 4. In some embodiments, p² is 2, 3, 4, or 5. In some embodiments, p² is 3 or 4. In some embodiments, p² is 3, 4, or 5. In some embodiments, p² is selected from the values represented in the compounds in Table 1.

As defined generally above, p³ is 0, 1, 2, 3, 4, or 5. In some embodiments, p³ is 0. In some embodiments, p³ is 1. In some embodiments, p³ is 2. In some embodiments, p³ is 3. In some embodiments, p³ is 4. In some embodiments, p³ is 5. In some embodiments, p³ is 0 or 1. In some embodiments, p³ is 0, 1, or 2. In some embodiments, p³ is 0, 1, 2, or 3. In some embodiments, p³ is 0, 1, 2, 3, or 4. In some embodiments, p³ is 1 or 2. In some embodiments, p³ is 1, 2, or 3. In some embodiments, p³ is 1, 2, 3, or 4. In some embodiments, p³ is 1, 2, 3, 4, or 5. In some embodiments, p³ is 2 or 3. In some embodiments, p³ is 2, 3, or 4. In some embodiments, p³ is 2, 3, 4, or 5. In some embodiments, p³ is 3 or 4. In some embodiments, p³ is 3, 4, or 5. In some embodiments, p³ is selected from the values represented in the compounds in Table 1.

As defined generally above, p⁴ is 0, 1, 2, 3, 4, or 5. In some embodiments, p⁴ is 0. In some embodiments, p⁴ is 1. In some embodiments, p⁴ is 2. In some embodiments, p⁴ is 3. In some embodiments, p⁴ is 4. In some embodiments, p⁴ is 5. In some embodiments, p⁴ is 0 or 1. In some embodiments, p⁴ is 0, 1, or 2. In some embodiments, p⁴ is 0, 1, 2, or 3. In some embodiments, p⁴ is 0, 1, 2, 3, or 4. In some embodiments, p⁴ is 1 or 2. In some embodiments, p⁴ is 1, 2, or 3. In some embodiments, p⁴ is 1, 2, 3, or 4. In some embodiments, p⁴ is 1, 2, 3, 4, or 5. In some embodiments, p⁴ is 2 or 3. In some embodiments, p⁴ is 2, 3, or 4. In some embodiments, p⁴ is 2, 3, 4, or 5. In some embodiments, p⁴ is 3 or 4. In some embodiments, p⁴ is 3, 4, or 5. In some embodiments, p⁴ is selected from the values represented in the compounds in Table 1.

As defined generally above, r¹ is 0, 1, 2, 3, 4, or 5. In some embodiments, r¹ is 0. In some embodiments, r¹ is 1. In some embodiments, r¹ is 2. In some embodiments, r¹ is 3. In some embodiments, r¹ is 4. In some embodiments, r¹ is 5. In some embodiments, r¹ is 0 or 1. In some embodiments, r¹ is 0, 1, or 2. In some embodiments, r¹ is 0, 1, 2, or 3. In some embodiments, r¹ is 0, 1, 2, 3, or 4. In some embodiments, r¹ is 1 or 2. In some embodiments, r¹ is 1, 2, or 3. In some embodiments, r¹ is 1, 2, 3, or 4. In some embodiments, r¹ is 1, 2, 3, 4, or 5. In some embodiments, r¹ is 2 or 3. In some embodiments, r¹ is 2, 3, or 4. In some embodiments, r¹ is 2, 3, 4, or 5. In some embodiments, r¹ is 3 or 4. In some embodiments, r¹ is 3, 4, or 5. In some embodiments, r¹ is selected from the values represented in the compounds in Table 1.

As defined generally above, r² is 0, 1, 2, 3, 4, or 5. In some embodiments, r² is 0. In some embodiments, r² is 1. In some embodiments, r² is 2. In some embodiments, r² is 3. In some embodiments, r² is 4. In some embodiments, r² is 5. In some embodiments, r² is 0 or 1. In some embodiments, r² is 0, 1, or 2. In some embodiments, r² is 0, 1, 2, or 3. In some embodiments, r² is 0, 1, 2, 3, or 4. In some embodiments, r² is 1 or 2. In some embodiments, r² is 1, 2, or 3. In some embodiments, r² is 1, 2, 3, or 4. In some embodiments, r² is 1, 2, 3, 4, or 5. In some embodiments, r² is 2 or 3. In some embodiments, r² is 2, 3, or 4. In some embodiments, r² is 2, 3, 4, or 5. In some embodiments, r² is 3 or 4. In some embodiments, r² is 3, 4, or 5. In some embodiments, r² is selected from the values represented in the compounds in Table 1.

As defined generally above, r³ is 0, 1, 2, 3, 4, or 5. In some embodiments, r³ is 0. In some embodiments, r³ is 1. In some embodiments, r³ is 2. In some embodiments, r³ is 3. In some embodiments, r³ is 4. In some embodiments, r³ is 5. In some embodiments, r³ is 0 or 1. In some embodiments, r³ is 0, 1, or 2. In some embodiments, r³ is 0, 1, 2, or 3. In some embodiments, r³ is 0, 1, 2, 3, or 4. In some embodiments, r³ is 1 or 2. In some embodiments, r³ is 1, 2, or 3. In some embodiments, r³ is 1, 2, 3, or 4. In some embodiments, r³ is 1, 2, 3, 4, or 5. In some embodiments, r³ is 2 or 3. In some embodiments, r³ is 2, 3, or 4. In some embodiments, r³ is 2, 3, 4, or 5. In some embodiments, r³ is 3 or 4. In some embodiments, r³ is 3, 4, or 5. In some embodiments, r³ is selected from the values represented in the compounds in Table 1.

As defined generally above, r⁴ is 0, 1, 2, 3, 4, or 5. In some embodiments, r⁴ is 0. In some embodiments, r⁴ is 1. In some embodiments, r⁴ is 2. In some embodiments, r⁴ is 3. In some embodiments, r⁴ is 4. In some embodiments, r⁴ is 5. In some embodiments, r⁴ is 0 or 1. In some embodiments, r⁴ is 0, 1, or 2. In some embodiments, r⁴ is 0, 1, 2, or 3. In some embodiments, r⁴ is 0, 1, 2, 3, or 4. In some embodiments, r⁴ is 1 or 2. In some embodiments, r⁴ is 1, 2, or 3. In some embodiments, r⁴ is 1, 2, 3, or 4. In some embodiments, r⁴ is 1, 2, 3, 4, or 5. In some embodiments, r⁴ is 2 or 3. In some embodiments, r⁴ is 2, 3, or 4. In some embodiments, r⁴ is 2, 3, 4, or 5. In some embodiments, r⁴ is 3 or 4. In some embodiments, r⁴ is 3, 4, or 5. In some embodiments, r⁴ is selected from the values represented in the compounds in Table 1.

In some embodiments, the present disclosure provides a compound of formula I, wherein Cy¹ is phenyl substituted with n instances of R¹, forming a compound of formula II:

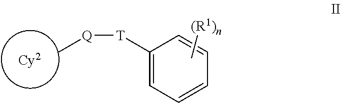

or a pharmaceutically acceptable salt thereof, wherein each of Cy², Q, R¹, T and n is as defined in embodiments and classes and subclasses herein.

In some embodiments, the present disclosure provides a compound of formula II wherein Q is —C(O)NH— or —NH—, forming a compound of formula III or IV:

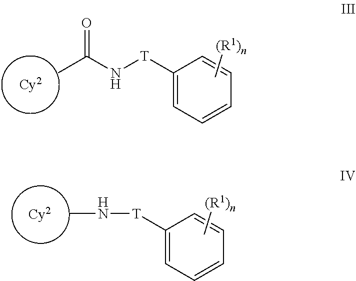

or a pharmaceutically acceptable salt thereof, wherein each of Cy², R¹, T and n is as defined in embodiments and classes and subclasses herein.

In some embodiments, the present disclosure provides a compound of formula III or IV, wherein T is selected from embodiments herein, forming a compound of formula V, VI, VII, VIII, IX, or X:

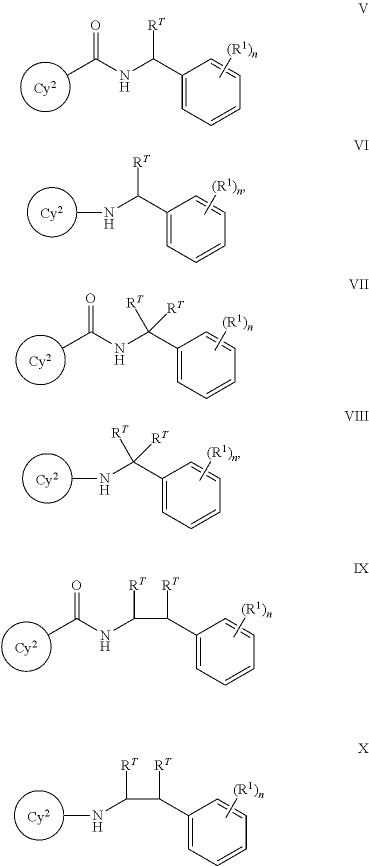

or a pharmaceutically acceptable salt thereof, wherein each of Cy², R¹, R^{T} and n is as defined in embodiments and classes and subclasses herein.

In some embodiments, the present disclosure provides a compound of formula V, wherein R^{T} is selected from embodiments herein, forming a compound of formula XI, XII, XIII, XIV, XV, XVI, XVII, XVIII, or XIX:

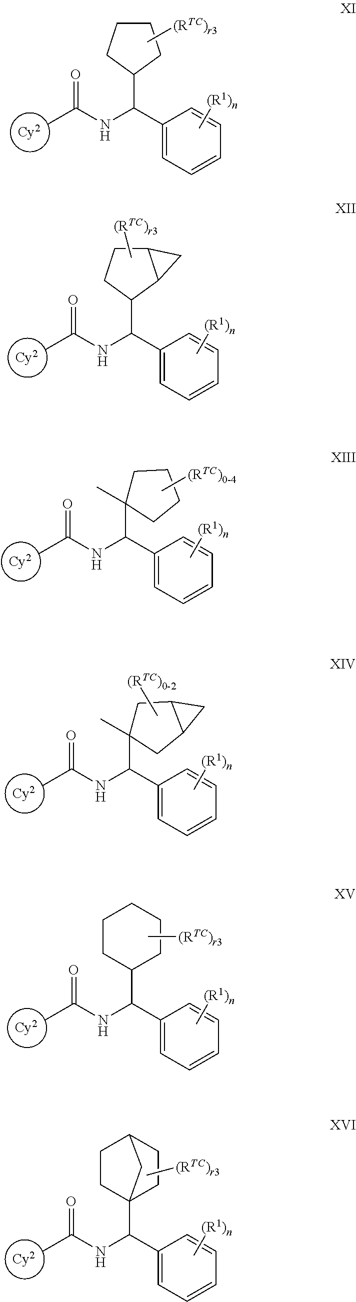

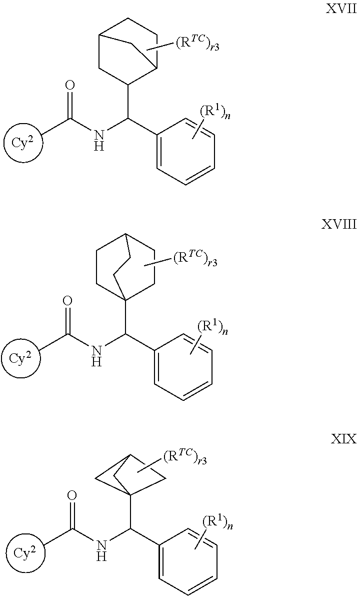

or a pharmaceutically acceptable salt thereof, wherein each of Cy², R¹, R^{TC}, n and r³ is as defined in embodiments and classes and subclasses herein.

In some embodiments, the present disclosure provides a compound of formula V, wherein Cy² is selected from embodiments herein, forming a compound of formula XX, XXI, XXII, XXIII, XXIV, or XXV:

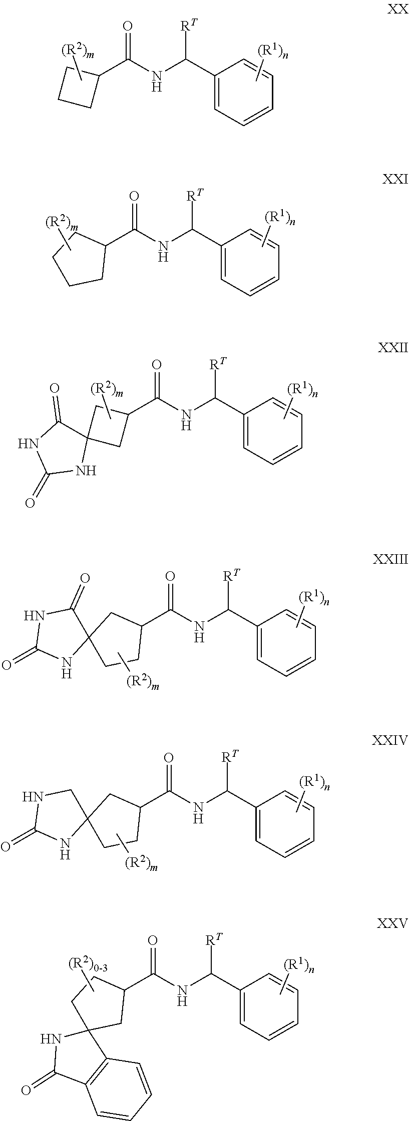

or a pharmaceutically acceptable salt thereof, wherein each of R¹, R², R^{T}, n and m is as defined in embodiments and classes and subclasses herein.

In some embodiments, the present disclosure provides a compound of formula V, wherein n and the position(s) of R¹ are selected from embodiments of Cy¹ herein, forming a compound of formulas XXVI, XXVII, XXVIII, XXIX, XXX, XXXI, XXXII, XXXIII, XXXIV, XXXV, and XXXVI:

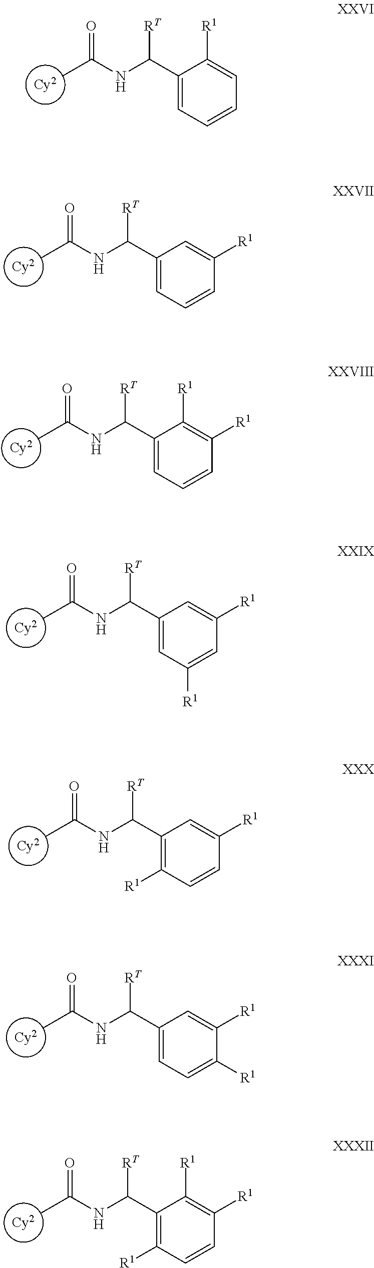

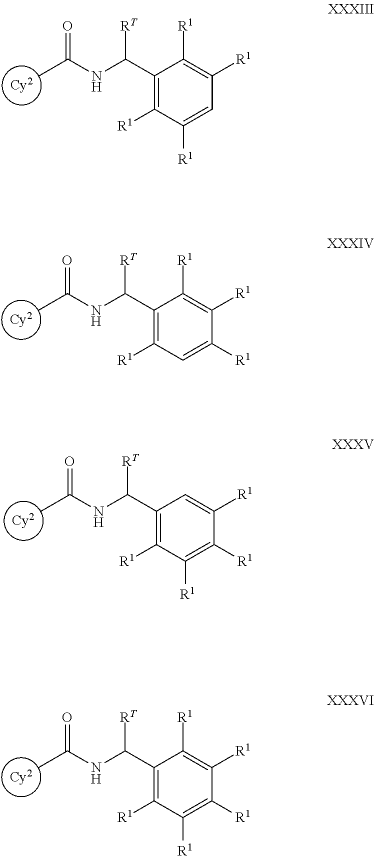

or a pharmaceutically acceptable salt thereof, wherein each of Cy², R¹, and R^{T} is as defined in embodiments and classes and subclasses herein.

In some embodiments, the present disclosure provides a compound of formula I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)C(O)—R^{2A}, —N(H)—R^{2A}, —CH₂—R^{2A}, or —R^{2A}.

In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)C(O)—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —N(H)—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —CH₂—R^{2A}. In some embodiments, the present disclosure provides a compound of I, II, III, IV, V, VI, VII, VIII, IX, X, XI, XII, XIII, XIV, XV, XVI, XVII, XVIII, XIX, XX, XXI, XXI, XXII, XXIII, XXIV, XXV, XXVI, XXVII, XXVIII, XXIX, XXX, XXXI, XXXII, XXXIII, XXXIV, XXXV, or XXXVI, wherein L¹ is a covalent bond, and R² is —R^{2A}.

Examples of compounds of the present disclosure include those listed in the Tables and exemplification herein, or a pharmaceutically acceptable salt, stereoisomer, or mixture of stereoisomers thereof. In some embodiments, the present disclosure provides a compound selected from those depicted in Table 1, below, or a pharmaceutically acceptable salt, stereoisomer, or mixture of stereoisomers thereof. In some embodiments, the present disclosure provides a compound set forth in Table 1, below, or a pharmaceutically acceptable salt thereof. In some embodiments, the present disclosure provides a compound set forth in Table 1, below.

Lengthy table referenced here
US20260035335A1-20260205-T00001
Please refer to the end of the specification for access instructions.

In chemical structures in Table 1, above, and the Examples, below, stereogenic centers are described according to the Enhanced Stereo Representation format (MDL/Biovia, e.g., using labels “or1”, “or2”, “abs”, “and1”). (See, for example, the structures of Compounds I-21, I-23, I-29, I-30, etc.)

In some embodiments, the present disclosure provides a compound in Table 1, above, wherein the compound is denoted as having an ADP-Glo IC₅₀ of “A”. In some embodiments, the present disclosure provides a compound in Table 1, above, wherein the compound is denoted as having an ADP-Glo IC₅₀ of “A” or “B”. In some embodiments, the present disclosure provides a compound in Table 1, above, wherein the compound is denoted as having an ADP-Glo IC₅₀ of “A” or “B” or “C”. In some embodiments, the present disclosure provides a compound in Table 1, above, wherein the compound is denoted as having an ADP-Glo IC₅₀ of “A” or “B” or “C” or “D”.

In some embodiments, the present disclosure provides a compound in Table 1, above, wherein the compound is denoted as having an MCF10A IC₅₀ of “A”. In some embodiments, the present disclosure provides a compound in Table 1, above, wherein the compound is denoted as having an MCF10A IC₅₀ of “A” or “B”. In some embodiments, the present disclosure provides a compound in Table 1, above, wherein the compound is denoted as having an MCF10A IC₅₀ of “A” or “B” or “C”. In some embodiments, the present disclosure provides a compound in Table 1, above, wherein the compound is denoted as having an MCF10A IC₅₀ of “A” or “B” or “C” or “D”.

In some embodiments, the present disclosure comprises a compound of formula I selected from those depicted in Table 1, above, or a pharmaceutically acceptable salt, stereoisomer, or mixture of stereoisomers thereof. In some embodiments, the present disclosure provides a compound of formula I selected from those depicted in Table 1, above, or a pharmaceutically acceptable salt thereof. In some embodiments, the present disclosure provides a compound of formula I selected from those depicted in Table 1, above.

In some embodiments, the present disclosure comprises a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above, or a pharmaceutically acceptable salt, stereoisomer, or mixture of stereoisomers thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above, or a pharmaceutically acceptable salt thereof. In some embodiments, the present disclosure provides a compound of formula II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, or XXXVI selected from those depicted in Table 1, above.

### 4. Uses, Formulation, and Administration

### Pharmaceutically Acceptable Compositions

According to another embodiment, the disclosure provides a composition comprising a compound of this disclosure, or a pharmaceutically acceptable derivative thereof, and a pharmaceutically acceptable carrier, adjuvant, or vehicle. In some embodiments, the disclosure provides a pharmaceutical composition comprising a compound of this disclosure, and a pharmaceutically acceptable carrier. The amount of compound in compositions of this disclosure is such that it is effective to measurably inhibit a PI3Kα protein kinase, or a mutant thereof, in a biological sample or in a patient. In certain embodiments, the amount of compound in compositions of this disclosure is such that it is effective to measurably inhibit a PI3Kα protein kinase, or a mutant thereof, in a biological sample or in a patient. In certain embodiments, a composition of this disclosure is formulated for administration to a patient in need of such composition. In some embodiments, a composition of this disclosure is formulated for oral administration to a patient.

The terms “subject” and “patient,” as used herein, mean an animal (i.e., a member of the kingdom animal), preferably a mammal, and most preferably a human. In some embodiments, the subject is a human, mouse, rat, cat, monkey, dog, horse, or pig. In some embodiments, the subject is a human. In some embodiments, the subject is a mouse, rat, cat, monkey, dog, horse, or pig.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of a PI3Kα protein kinase, or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vagina to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Preferably, pharmaceutically acceptable compositions of this disclosure are formulated for oral administration. Such formulations may be administered with or without food. In some embodiments, pharmaceutically acceptable compositions of this disclosure are administered without food. In other embodiments, pharmaceutically acceptable compositions of this disclosure are administered with food.

The amount of compounds of the present disclosure that may be combined with the carrier materials to produce a composition in a single dosage form will vary depending upon the patient treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The precise dose to be employed in the compositions will also depend on the route of administration and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range and may be combined with any other dosage to constitute a dosage range comprising an upper and lower limit.

In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient.

Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Ale^{YA}nder T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which is hereby incorporated by reference in its entirety).

The pharmaceutically acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sufate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cationic surfactants, such as cetyltrimethylammonium bromide; amine salts of formula N⁺R′R″R′″, in which the R radicals are identical or different optionally hydroxylated hydrocarbon radicals; cationic surfactants, such as octadecylamine hydrochloride; non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide; and amphoteric surfactants, such as substituted lauryl compounds of betaine.

Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain wetting or emulsifying agents, or pH buffering agents.

Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

### Uses of Compounds and Pharmaceutically Acceptable Compositions

Compounds and compositions described herein are generally useful for the inhibition of a kinase or a mutant thereof. In some embodiments, the kinase inhibited by the compounds and compositions described herein is a phosphatidylinositol 3-kinase (PI3K). In some embodiments, the kinase inhibited by the compounds and compositions described herein is one or more of a PI3Kα, PI3Kδ, and PI3Kγ. In some embodiments, the kinase inhibited by the compounds and compositions described herein is a PI3Kα. In some embodiments, the kinase inhibited by the compounds and compositions described herein is a PI3Kα containing at least one of the following mutations: H1047R, E542K, and E545K.

Compounds or compositions of the disclosure can be useful in applications that benefit from inhibition of PI3K enzymes. For example, PI3K inhibitors of the present disclosure are useful for the treatment of cellular proliferative diseases generally. Compounds or compositions of the disclosure can be useful in applications that benefit from inhibition of PI3Kα enzymes. For example, PI3Kα inhibitors of the present disclosure are useful for the treatment of cellular proliferative diseases generally.

Aberrant regulation of PI3K, which often increases survival through Aid activation, is one of the most prevalent events in human cancer and has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the p110 alpha isoform, PIK3CA, and for Akt are amplified, and increased protein expression of their gene products has been demonstrated in several human cancers. Furthermore, mutations and translocation of p85 alpha that serve to up-regulate the p85-p110 complex have been described in human cancers. Finally, somatic missense mutations in PIK3CA that activate downstream signaling pathways have been described at significant frequencies in a wide diversity of human cancers (Kang et el., Proc. Natl. Acad. Sci. USA 102:802 (2005); Samuels et al., Science 304:554 (2004); Samuels et al., Cancer Cell 7:561-573 (2005)). These observations show that deregulation of phosphoinositol-3 kinase, and the upstream and downstream components of this signaling pathway, is one of the most common deregulations associated with human cancers and proliferative diseases (Parsons et al., Nature 436:792 (2005); Hennessey at el., Nature Rev. Drug Disc. 4:988-1004 (2005)).

The activity of a compound utilized in this disclosure as an inhibitor of a PI3K kinase, for example, a PI3Kα, or a mutant thereof, may be assayed in vitro, in vivo or in a cell line. In vitro assays include assays that determine inhibition of either the phosphorylation activity and/or the subsequent functional consequences, or ATPase activity of an activated PI3Kα, or a mutant thereof. Alternative in vitro assays quantitate the ability of the inhibitor to bind to a a PI3Kα. Inhibitor binding may be measured by radiolabeling the inhibitor prior to binding, isolating the inhibitor/PI3Kα complex and determining the amount of radiolabel bound. Alternatively, inhibitor binding may be determined by running a competition experiment where new inhibitors are incubated with a PI3Kα bound to known radioligands. Representative in vitro and in vivo assays useful in assaying a PI3Kα inhibitor include those described and disclosed in the patent and scientific publications described herein. Detailed conditions for assaying a compound utilized in this disclosure as an inhibitor of a PI3Kα, or a mutant thereof, are set forth in the Examples below.

### Treatment of Disorders

Provided compounds are inhibitors of PI3Kα and are therefore useful for treating one or more disorders associated with activity of PI3Kα or mutants thereof. Thus, in certain embodiments, the present disclosure provides a method of treating a PI3Kα-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a PI3Kα-mediated disorder in a subject comprising administering a therapeutically effective amount of a compound of the present disclosure, or a pharmaceutically acceptable composition thereof, to a subject in need thereof. In some embodiments, the subject has a mutant PI3Kα. In some embodiments, the subject has PI3Kα containing at least one of the following mutations: H1047R, E542K, and E545K. In some embodiments, the subject has PI3Kα containing at least one of the mutations in Table A:

TABLE A

M1*
M1I
M1L
M1R
M1T
M1V
P2H
P2L
P2S
P3A
P3L
P3S
P3T
R4*
R4G
R4L
R4P
R4Q
R4_P17del
R4_P18del
P5T
P5_R19del
S6L
S6_I13del
S7*
S7L
S7_P18del
S7_R19del
S7fs*3
G8C
G8D
G8R
G8S
G8V
G8_P17del
G8_P18del
G8_R19del
E9*
E9A
E9G
E9K
E9Q
E9_I20 > V
E9_L15 > G
E9_M16 > V
E9_M16del
E9_P17 > A
E9_P17del
E9_P18 > CPT
E9_P18 > GCPT
E9_P18del
E9_R19 > G
E9_R19del
E9fs*6
L10P
L10Q
L10R
L10V
L10_L15 > R
L10_L15del
L10_L21del
L10_M16del
L10_P17 > T
L10_P17del
L10_P18 > Q
L10_P18del
W11*
W11C
W11G
W11L
W11R
W11S
W11_H14del
W11_L15del
W11_L21del
W11_M16del
W11_P17 > S
W11_P18 > S
W11_P18 > CG
W11_P18del
W11_R19del
W11_V22del
W11del
G12C
G12D
G12S
G12V
G12_L21del
G12_P17 > A
G12_P18 > I
I13F
I13M
I13N
I13V
I13_P18del
I13del
H14D
H14N
H14Y
H14_I20del
H14_L21del
L15S
L15V
L15W
M16I
M16K
M16L
M16R
M16T
M16V
P17A
P17L
P17R
P17S
P18L
P18Q
P18S
P18T
P18fs*4
R19*
R19I
R19K
I20M
I20T
I20fs*3
L21I
L21R
L21V
V22G
V22I
V22L
E23K
E23Q
C24F
C24Y
L25*
L25F
L25S
P27S
G29K
G29R
M30I
M30V
M30fs*9
I31M
I31V
V32A
V32L
V32M
T33A
T33I
T33S
E35*
E35K
E35Q
C36*
L37F
L37I
R38C
R38G
R38H
R38L
R38S
R38_143 > L
E39*
E39A
E39D
E39K
E39Q
A40V
T41I
L42*
L42F
I43L
I43M
T44F
T441
I45L
I45T
I45V
K46M
H47L
H47Q
H47Y
E48*
E48K
E48Q
E48V
F50L
F50fs*22
K51R
E52*
E52K
E52Q
A53S
A53V
A53fs*19
R54I
R54K
K55I
K55Q
Y56*
Y56H
P57H
P57L
P57S
L58F
L58R
L58V
L58fs*13
L58fs*14
H59L
H59N
H59R
H59Y
Q60*
Q60E
Q60L
Q60P
L61F
L61I
L61R
L62F
L62I
L62del
Q63*
Q63E
Q63H
Q63L
Q63fs*9
D64G
D64H
D64N
D64V
D64Y
D64_S72 > VL
E65*
E65A
E65G
E65K
E65V
S66C
S66F
S66Y
S67A
S67C
S67F
S67Y
S67del
Y68C
Y68H
I69F
I69L
I69N
I69S
I69V
F70V
V71G
V71I
V71L
S72G
S72R
S72T
S72fs*27
V73A
V73I
T74A
T74I
T74S
Q75*
Q75E
Q75H
Q75L
Q75P
E76Q
E76del
A77G
A77P
E78*
E78G
E78K
E78Q
E78_R79insVSK
NTYLSKCYS
R79M
R79W
R79_E80 > K
E80K
E80_E81 > KK
E81*
E81A
E81D
E81G
E81K
E81Q
E81V
E81_F82 > V
E81del
F82I
F82L
F83C
F83I
F83K
F83L
F83S
F83V
F83Y
F83del
F83fs*17
D84H
D84N
D84Y
D84fs*1
E85*
E85K
E85Q
T86I
T86S
R87G
R871
R87S
R87T
R88*
R88G
R88L
R88P
R88Q
L89F
L89H
C90F
C90G
C90S
C90W
C90Y
C90fs*1
L92F
L92I
R93G
R93L
R93L
R93P
R93Q
R93W
L94F
F95C
F95S
F95fs*2
Q96*
Q96E
Q96H
Q96K
Q96R
Q96fs*4
P97F
P97H
P97L
P97S
F98C
F98L
F98V
L99*
L99F
L99V
L99fs*1
K100*
K100N
K100del
V101A
V101I
V101L
V101del
I102F
I102M
I102T
I102V
I102_E103 > K
I102_E103ins16
I102_P104 > K
I102_P104 > T
I102_P104del
I102_V105del
I102del
I102fs*6
E103*
E103D
E103G
E103K
E103Q
E103_E110del
E103_G106 > D
E103_N107del
E103_P104 > A
E103_P104 > S
E103_P104del
E103_R108 > YC
E103_V105 > A
E103_V105 > D
E103del
P104A
P104K
P104L
P104Q
P104R
P104S
P104T
P104_E110 > Q
P104_E110del
P104_G106 > R
P104_G106 > S
P104_G106del
P104_N107 > H
P104_N107del
P104_V105 > I
P104_V105 > L
P104_V105del
P104_V105insVGNREEKILNREIGMIQY
P104del
V105A
V105I
V105L
V105_E109 > A
V105_E109 > K
V105_E109 > EE
V105_E109del
V105_E110del
V105_G106del
V105_N107 > D
V105_N107 > Y
V105_N107 > AT
V105_N107 > GD
V105_N107del
V105_N107del
V105_R108del
V105del
V105del
G106A
G106C
G106D
G106E
G106F
G106L
G106R
G106S
G106V
G106_E109 > A
G106_E109 > E
G106_E109del
G106_E110 > K
G106_I112 > F
G106_K111 > E
G106_N107 > T
G106_N107del
G106_R108 > I
G106_R108 > V
G106_R108del
G106del
N107H
N107I
N107K
N107S
N107T
N107Y
N107_E109 > K
N107_E109del
N107_E109del
N107_E110del
N107_K111 > YRE
N107_R108 > S
N107_R108del
N107del
R108C
R108G
R108H
R108L
R108P
R108S
R108_E109 > C
R108_E109 > Q
R108_E109del
R108_E109insLKVIEPVGNR
R108_E110del
R108_I112 > L
R108_I112 > V
R108_I112del
R108_K111 > Q
R108_K111 > EA
R108_K111del
R108del
E109*
E109A
E109G
E109K
E109_E110 > A
E109_I112 > D
E109_I112 > V
E109_L113 > D
E109_L113 > V
E109_N114del
E109_R115 > G
E109fs*11
E110*
E110K
E110Q
E110_E116del
E110_I112 > D
E110_I112del
E110_K111 > D
E110_K111 > G
E110_K111 > M
E110_K111 > V
E110_K111del
E110_K111insE
E110_N114 > D
E110_R115 > G
E110del
K111E
K111M
K111N
K111Q
K111R
K111T
K111_E116del
K111_I112 > D
K111_I112 > F
K111_I112 > N
K111_I112del
K111_I112insK
K111_I112insEK
K111_L113 > I
K111_L113del
K111_N114 > D
K111_N114del
K111_R115del
K111del
I112D
I112F
I112L
I112N
I112S
I112T
I112V
I112_L113del
I112_L113insl
I112_L113insQI
I112 > MSM
I112del
L113F
L113H
L113I
L113R
L113V
L113_I117del
L113_N114 > H
L113_N114del
L113_N114insL
L113del
N114D
N114H
N114I
N114S
N114Y
N114_I117del
N114_R115 > K
N114_R115insLN
N114del
R115*
R115E
R115G
R115L
R115P
R115Q
R115_E116insDEEKILNR
R115del
E116*
E116G
E116K
E116Q
E116_I117insRE
I117F
I117V
I117_G118insEI
G118D
G118E
G118V
G118_F119insMIEPVGNREEKILNREIG
G118_F119insMIQEILNREIG
G118_F119insMIQYPQSILNREIG
F119I
F119L
A120S
A120T
I121L
I121N
G122A
G122C
G122D
G122S
G122V
M123I
M123K
M123L
M123V
P124A
P124L
P124Q
P124S
P124_V125 > Q
V125A
V125E
V125G
V125L
V125M
C126S
C126W
C126Y
C126fs*19
E127*
E127D
E127K
E127Q
F128Y
D129H
D129N
D129Y
M130I
M130T
V131F
V131I
K132T
D133G
D133H
D133Y
D133fs*12
P134L
P134S
E135*
E135K
E135Q
E135fs*3
Q137*
Q137H
Q137L
D138G
D138N
D138Y
F139I
F139L
F139S
F139Y
F139_R140 > S*
R140*
R140Q
R141I
R141K
R141T
R141fs*4
N142D
I143T
L144P
L144V
N145K
N145S
N145T
N145Y
N145_V146 > KI
V146A
V146G
V146I
C147F
C147S
C147Y
K148E
E149*
E149A
E149D
E149G
E149K
E149Q
E149V
A150G
A150S
A150V
V151A
V151L
V151M
D152H
D152N
L153F
R154K
R154M
R154S
R154W
D155H
D155V
D155Y
L156I
L156V
N157K
S158*
S158A
S158L
S158P
P159S
P159T
H160N
H160R
H160Y
H160fs*12
S161C
S161I
S161T
R162G
R162I
R162K
R162T
A163G
A163T
A163V
M164I
M164L
M164T
M164V
Y165C
Y165F
YV165_166*F
V166A
V166G
V166I
Y167*
Y167C
Y167H
P168F
P168H
P168L
P168R
P168S
P168T
P169A
P169L
P169Q
P169R
P169S
P169T
N170fs*2
V171E
V171L
E172D
E172G
E172Q
S173C
S173Y
S174L
P175L
P175Q
P175S
E176D
E176K
E176Q
L177*
L177F
P178S
K179N
K179Q
H180N
H180P
H180R
I181V
Y182F
Y182H
N183H
N183T
K184E
K184N
K184Q
K184R
K184T
L185*
L185F
L185S
D186A
D186E
D186G
D186N
D186Y
K187E
K187Q
K187R
G188R
Q189*
Q189fs*14
I190L
I190V
V192L
V192M
V193L
I194M
W195*
W195C
W195R
V196L
I197M
I197T
I197V
V198D
V198F
V198I
S199F
S199P
S199Y
P200A
P200L
P200R
P200S
N201D
N201S
N202D
N202fs*9
D203G
D203H
D203N
D203Y
K204T
Q205*
Q205H
Q205K
Q205P
Q205R
K206E
Y207S
T208A
L209V
K210R
I211M
I211V
N212S
H213P
H213Y
D214E
D214G
D214Y
D214_C215 > ER
C215S
C215T
C215Y
V216A
V216I
V216L
P217L
P217R
P217S
E218D
E218K
E218Q
E218fs*4
Q219H
Q219K
V220L
I221N
I221V
E223D
E223G
E223K
E223Q
A224G
A224S
A224T
A224V
I225M
I225S
I225T
I225V
R226G
R226S
R226T
K227*
K228N
T229A
T229P
T229S
T229fs*9
T229fs*11
R230*
R230G
R230L
R230Q
R230fs*7
S231R
M232I
M232L
M232V
L233F
L233S
L233V
L234fs*8
S235C
S235F
S235P
S235T
S236C
S236F
S236T
S236Y
E237K
E237Q
Q238K
Q238L
L239I
L239Q
L239R
L239V
K240N
K240Q
K240T
V243D
V243G
V243I
V243L
V243fs*15
E245K
Y246C
Q247*
Q247H
Q247L
Q247R
G248C
G248V
K249*
K249N
K249T
Y250C
Y250F
Y250H
Y250N
I251N
K253I
K253N
K253T
V254G
V254L
V254M
V254_C255 > LW
C255F
C255Y
C255fs*3
G256*
G256A
G256E
G256R
G256V
C257R
D258E
D258G
D258H
D258N
E259Q
Y260C
Y260H
F261L
F261V
F261Y
E263*
E263D
E263K
E263Q
E263fs*5
K264Q
K264T
K264fs*4
Y265*
Y265C
P266H
P266L
P266R
P266S
L267M
L267P
L267V
S268N
S268T
Q269*
Q269H
Q269L
Q269R
Q269fs*4
Y270F
Y270N
K271N
K271R
Y272C
Y272F
Y272H
I273L
I273M
I273T
R274G
R274I
R274K
R274T
S275C
S275I
S275N
C276*
C276F
C276G
I277L
I277T
I277V
I277fs*7
M278I
M278K
M278L
M278T
M278fs*23
L279F
L279H
L279I
L279R
L279V
G280A
G280E
G280K
G280R
G280V
G280W
R281M
R281S
R281fs*5
M282I
P283L
P283S
N284H
N284K
N284S
N284Y
L285F
L285M
M286I
L287F
M288I
M288T
A289T
A289V
K290N
E291*
E291K
E291Q
S292C
S292I
S292N
S292R
L293F
Y294*
Y294H
Y294fs*25
S295A
S295C
S295F
S295Y
Q296*
Q296E
Q296K
Q296R
L297P
L297R
P298A
P298Q
P298R
P298S
M299I
M299L
M299T
D300A
D300E
D300H
D300N
C301*
C301F
C301G
C301S
C301W
C301Y
F302C
F302Y
T303A
T303K
T303R
M304I
M304L
M304T
M304V
P305S
P305_N319 > H
S306A
S306C
S306F
S306P
S306Y
S306fs*13
Y307F
Y307H
S308A
S308C
S308F
S308Y
R309G
R309_R310 > S
R310C
R310H
R310L
I311F
I311N
S312C
S312F
S312fs*18
T313I
T313K
T315I
P316L
P316Q
P316S
P316T
Y317C
Y317F
Y317H
M318I
M318T
M318V
M318fs*15
N319S
N319T
G320*
G320A
G320E
G320R
G320V
E321A
E321K
E321Q
E321V
S323A
S323C
S323F
S323P
S323Y
T324I
K325E
K325Q
K325R
K325_I330del
K325fs*6
S326A
S326C
S326F
S326P
S326Y
L327F
L327H
L327I
L327fs*4
W328*
W328C
W328L
W328R
W328S
V329F
V329G
V329I
I330L
I330V
N331H
S332I
S332T
A333S
A333T
A333V
L334F
R335I
R335K
R335S
R335T
R335_I336ins18
R335fs*2
R335fs*17
R335fs*33
I336M
I336fs*8
K337Q
K337T
I338F
I338N
I338S
I338T
I338fs*7
L339F
L3391
L339R
L339V
C340F
C340R
C340_A341insVKILC
A341S
A341V
A341_T342insIKILCA
A341_T342insLRIKILCA
A341_T342insYKILCA
T342A
T342I
T342S
T342_N345 > H
T342_Y343ins37
T342_Y343insRIKILCAT
Y343C
Y343F
Y3431
Y343L
Y343S
Y343_N345del
Y343_V344 > L
Y343_V344insATY
Y343_V344insERIKILCATY
Y343_V344insLCATY
V344A
V344E
V344F
V344G
V344L
V344M
V344R
V344_N345 > RFSAFWLRSS
V344_N345insK
V344_N345insM
V344_N345insV
V344_N345insILCATYV
V344_N345insKV
V344_N345insKILCATYV
V344_N345insTTYV
V344_N345insTYV
V344_N347del
N345D
N345H
N345I
N345K
N345S
N345T
N345Y
N345_I348 > K
N345_I348del
N345_K353del
N345_V346 > K
N345_V346 > KL
N345_V346 > KATYVNV
N345_V346insATYVN
V346A
V346E
V346G
V346L
V346L
V346Q
V346_N347 > ERTYVNVN
V346_N347insK
V346_N347insV
V346_N347insEKIKKKKKK
V346_N347insKNV
V346_N347insMNV
V346_N347insVNV
V346 > GK
N347D
N347I
N347K
N347T
N347Y
N347_I348insR
N347_I348insVN
I348M
I348S
I348V
I348_R349insLNI
R349*
R349Q
R349_D350insIR
R349_D350insVR
D350G
D350H
D350K
D350N
D350Y
D350_I351insKKILCATYVNVNIRD
D350_I351insRIKILCATYVNVNIRD
D350del
I351F
I351S
I351_D352 > E
I351_D352 > KYLQ
I351_D352insGIKILCATYVNVNIRDI
I351_D352insVNVNIRDI
D352H
D352N
D352Y
D352 > RDIN
K353M
K353N
K353_I354insVVNVNIRDIDK
I354D
I354F
I354L
I354N
I354S
I354T
I354V
Y355C
Y355_V356insY
V356A
V356F
V356I
V356L
R357*
R357G
R357L
R357Q
R357fs*10
T358A
T358K
T358S
T358_G359insA
G359A
G359C
G359R
G359V
I360F
I360T
I360V
Y361C
Y361F
Y361H
Y361_H362insQIYVRTGIY
H362N
H362R
H362Y
G363A
G363E
G363V
G363_G364insYVRTGIYHG
G363 > YHR
G364E
G364K
G364R
G364_E365insVRTGIYHGG
E365D
E365K
E365Q
E365V
P366F
P366H
P366L
P366R
P366S
P366T
P366fs*5
C368Y
D369G
D369N
D369Y
N370D
N370K
N370S
N372S
T373I
T373P
T373S
Q374*
Q374E
Q374H
R375G
R375I
R375K
R375S
V376I
P377L
P377S
C378F
C378L
C378R
C378W
C378Y
S379C
S379F
N380K
N380Y
P381A
P381S
R382K
R382W
R382fs*6
W383*
W383C
W383L
E385K
W386R
L387Q
L387V
N388D
N388I
N388T
Y389C
Y389F
Y389S
D390A
D390H
D390N
D390Y
I391V
I391fs*36
Y392*
Y392H
P394S
P394_D395ins23
D395H
D395N
D395V
D395Y
L396F
L396I
L396P
L396V
P397A
P397R
P397S
P397T
R398C
R398H
R398L
A399D
A399G
A399S
A399T
A399V
R401*
R401L
R401Q
R401S
L402V
C403R
L404F
L404I
L404V
S405F
S405T
S405Y
I406F
I406M
I406V
C407F
C407R
C407W
C407Y
C407fs*21
S408C
S408P
V409F
V409I
K410_G411insGRKGAKEVKYFRRK
K410fs*6
G411D
G411R
G411S
G411V
R412*
R412L
R412Q
K413N
K413_G414insRK
G414A
G414D
G414R
G414S
G414V
G414fs*13
A415D
K416E
K416I
E417D
E417G
E417K
E417Q
E417V
E418*
E418A
E418K
E418Q
E418_C420 > D
E418_P421 > A
H419L
H419P
H419Q
H419R
H419Y
H419_C420 > R
H419_C420del
H419_L422 > T
H419_L422 > LM
H419_L422 > PW
H419_L422del
H419_L422del
H419_P421 > L
H419_P421 > P
H419_P421 > Q
H419_P421 > R
H419_P421 > T
H419_P421del
H419fs*11
C420R
C420S
C420Y
C420_P421del
C420_L422 > W
C420_A423 > W
C420_A423 > Y
C420_A423del
C420_I427 > WHGNV
P421A
P421L
P421R
P421S
P421T
P421_A423 > H
P421_A423 > H
P421_A423del
P421_L422del
P421 > RR
L422E
L422F
L422S
L422W
L422_A423 > F
A423E
A423S
A423T
A423V
W424*
W424C
W424G
W424L
W424R
W424_G425insF
W424_I427del
G425E
G425R
G425V
N426D
N426S
N426fs*6
I427K
I427M
I427T
I427V
N428K
N428S
N428Y
L429F
L429V
L429fs*2
F430C
F430L
Y432F
Y432H
Y432fs*5
T433A
T433R
T433_D434 > NTD
T433_D434insTLVSGKMALNLWPVPHGLE
D434E
D434H
D434fs*2
T435I
T435N
T435S
L436P
L436V
L436fs*1
L436fs*32
V437E
V437G
V437I
S438A
S438C
S438fs*30
G439A
G439E
G439K
G439R
G439fs*5
K440E
K440N
K440fs*45
M441I
M441V
E441fs*3
M441fs*3
M441fs*28
A442T
A442V
L443F
N444H
N444K
N444_G451 > K
N444_L455 > H
L445F
L445I
L445_W446insL
W446*
W446S
W446_D454del
W446_E453del
W446_G451del
W446_G460 > C
W446_H450 > R
W446_H450del
W446_I459del
W446_L456del
W446_P447insW
W446_P458del
W446_V461del
P447A
P447L
P447S
P447_L452del
P447_L455del
P447_V448ins24
P447_V448insLFDYTDTLVSGKMALNLWP
V448A
V448E
V448G
V448L
V448_D454del
V448_E453 > K
V448_E453 > P
V448_E453 > YK
V448_E453del
V448_G451del
V448_L452del
V448_L455del
V448_P449insSGKMALNLWPV
V448_P449insVSGKMALNLWPV
V448fs*14
P449A
P449H
P449L
P449R
P449S
P449T
P449_D454 > R
P449_D454del
P449_E453 > Q
P449_E453del
P449_H450insLVSGKMALNLWPVP
P449_H450insPVP
P449_H450insPVP
P449_I459del
P449_L452del
P449_L455del
P449_L456del
P449_P458del
H450D
H450N
H450Q
H450R
H450Y
H450_D454del
H450_E453del
H450_G451 > PRG
H450_G460 > R
H450_I459 > L
H450_I459del
H450_L452del
H450_L455 > P
H450_L455 > Q
H450_L455 > KM
H450_L455del
H450_L455del
H450_L455del
H450_L456 > P
H450_L456del
H450_N457del
H450_P458 > LIH
H450_P458del
H450_V461 > GS
G451A
G451E
G451K
G451R
G451V
G451_D454 > RR
G451_D454del
G451_E453del
G451_G460del
G451_I459 > A
G451_I459 > V
G451_L452 > KKKKK
G451_L452insFGKMALNLWPVPHG
G451_L455 > A
G451_L455 > V
G451_L455 > GTM
G451_L455del
G451_L456 > K
G451_L456 > V
G451_N457del
G451_P458 > V
G451_P458del
L452S
L452_E453del
L452_E453ins21
L452_E453insAGKMALNLWPVPHGL
L452_E453insVSGKMALNLWPVPHGL
L452_G460 > F
L452_G460del
L452_I459 > FRRF
L452_I459 > PLWARL
L452_I459del
L452_I459del
L452_L455 > W
L452_N457 > T
L452_N457 > Y
L452_P458 > F
L452_T462 > QKT
L452_V461 > F
E453*
E453A
E453D
E453G
E453K
E453Q
E453V
E453_D454 > KN
E453_D454del
E453_D454insGKMALNLWPVPHGLE
E453_D454insVSGKMALNLWPVPHGLE
E453_D454insVVSGKMALNLWPVPHGLE
E453_G460 > C
E453_G460del
E453_G463del
E453_I459 > G
E453_I459 > V
E453_I459del
E453_L455 > G
E453_L455 > V
E453_L455del
E453_L455del
E453_L456 > M
E453_L456 > V
E453_L456del
E453_P458del
E453_T462del
E453_T462del
E453 > GLK
E453del
D454E
D454G
D454H
D454K
D454N
D454Y
D454_1459del
D454_K468del
D454_L455 > V
D454_L455del
D454_N467 > VS
D454_P458 > Y
D454del
L455F
L455_G460 > C
L455_G460del
L455_I459 > C
L455_I459 > C
L455_I459 > F
L455_I459del
L455_L456 > FM
L455_L456insPGKMALNLWPVPHGLEDL
L455_N467 > SD
L455_N467del
L455_P458del
L455_T462del
L455_V461 > F
L456M
L456P
L456R
L456V
L456_I459del
L456_N457insKKKKKKREDLL
N457D
N457K
N457S
N457_G460 > S
N457_G463 > R
N457_I459 > K
N457_I459 > K
N457_P458 > TR
N457_T462del
N457_V461del
P458A
P458L
P458R
P458S
P458_G463 > R
P458_I459insMNLWPVPHGLEDLLNP
P458_K468del
P458_V461 > L
I459M
I459N
I459S
I459T
I459V
I459_T462del
G460A
G460C
G460D
G460R
G460V
V461A
V461_N465del
T462I
T462_N465del
T462_S464del
T462fs*12
G463*
G463A
G463E
G463R
G463V
G463_K468del
S464*
S464L
N465I
N465K
N465S
N465T
N465Y
N465_P466ins27
P466L
P466Q
P466S
P466_N467insKLLNPIGVTGSNP
N467H
N467K
N467T
K468*
K468R
K468T
K468_E469ins31
K468_E469ins35
K468_E469insVERLLNPIGVTGSNPNK
K468_E469insVLLNPIGVTGSNPNK
E469*
E469A
E469D
E469G
E469K
E469V
E469_T470 > D
E469 > DK
T470I
T470N
T470P
T470S
T470fs*4
P471A
P471I
P471L
P471Q
P471S
P471T
P471 > QTL
C472S
C472W
C472 > FF
L473I
L473V
L473_E474insL
L473_E474insACL
L473_L475 > FGVWSLEL
E474A
E474K
E474Q
E474V
L475*
L475F
E476G
E476K
E476Q
E476_F477insLE
D478A
D478E
D478G
D478H
D478N
D478Y
W479C
W479S
F480L
S481G
S481N
S481R
S481T
S482C
S482N
V483A
V483L
V483M
V484A
V484I
V484L
K485E
K485R
K485T
F486L
F486Y
P487L
P487Q
P487R
P487S
D488G
D488H
D488N
D488_S490del
M489I
M489V
S490P
V491G
V491L
V491M
I492F
I492M
I492T
E493K
E493Q
E494*
E494D
E494K
E494Q
E494V
H495L
H495N
H495Q
A496D
A496S
A496T
A496V
N497S
W498*
W498C
W498L
W498R
W498S
S499F
S499Y
V500L
V500fs*9
S501F
S501T
S501Y
R502*
R502G
R502Q
E503G
E503K
E503Q
E503del
G505A
G505E
G505R
F506C
F506L
F506V
S507G
S507I
S507R
S507T
Y508C
Y508H
Y508N
S509C
S509F
H510Q
H510R
H510Y
A511P
A511S
G512*
G512A
G512E
G512R
G512V
L513P
L513V
L513fs*5
S514C
S514N
S514R
N515Y
R516I
R516K
R516T
L517I
L517P
L517R
L517V
A518G
A518P
A518S
A518T
R519K
R519T
D520E
D520H
D520N
N521D
N521K
N521S
E522*
E522K
E522Q
L523F
L523V
L523fs*1
R524K
R524M
R524S
R524fs*36
E525*
E525A
E525G
E525K
E525fs*35
N526S
N526fs*34
D527E
D527G
D527H
D527N
K528E
E529K
E529Q
Q530*
Q530H
Q530K
Q530R
L531R
A533T
I534N
I534V
S535C
S535F
S535Y
T536A
T536K
T536S
R537*
R537L
R537Q
D538A
D538E
D538G
D538H
D538N
D538Y
D538_S541 > A
D538del
P539A
P539H
P539L
P539R
P539S
P539T
P539del
L540F
L540H
L540I
L540P
L540R
L540V
S541A
S541C
S541F
S541L
S541P
S541T
S541_E542insAISTRDRLS
S541fs*1
E542A
E542D
E542G
E542I
E542K
E542L
E542Q
E542R
E542V
I543V
I543_E545del
T544N
T544S
E545A
E545D
E545G
E545K
E545L
E545N
E545P
E545Q
E545R
E545S
E545T
E545V
E545W
E545_Q546 > DK
E545_Q546 > DL
Q546E
Q546H
Q546K
Q546L
Q546P
Q546R
E547*
E547D
E547G
E547K
E547Q
K548N
K548Q
K548R
D549G
D549H
D549N
D549Y
D549fs*21
F550C
F550L
F550V
L551I
L551P
L551V
L551fs*8
L551fs*9
W552*
W552C
W552G
W552R
S553C
S553G
S553N
S553R
S553T
H554D
H554Q
H554R
H554Y
R555G
R555K
R555T
H556D
Y557C
Y557S
C558F
C558S
V5591
V559L
T560I
T560P
T560S
I561M
I561V
P562L
P562S
E563D
E563K
I564S
P566L
P566S
K567Q
L5691
L570M
L570P
S571C
V572A
V572I
V572fs*9
K573R
W574L
N575I
N575K
S576F
S576T
S576Y
R577G
R577I
R577K
R577S
R577T
D578E
D578H
D578N
D578V
D578Y
E579*
E579D
E579K
E579Q
V580A
V580E
V580L
A581T
A581V
Q582*
Q582L
Q582R
M583I
Y584H
C585F
C585G
L586F
L586M
V587I
V587fs*10
K588N
K588fs*8
D589E
D589H
D589Y
W590*
P591T
P592A
P592L
P592S
P592T
P592fs*32
I593M
I593N
I593V
P595L
P595T
E596K
E596Q
E596V
E596fs*28
Q597H
Q597K
Q597R
Q597_A598 > HT
A598D
A598T
M599L
M599_E600 > IK
E600*
E600A
E600K
L602M
L602R
L602_D603insT
D603N
D603Y
C604R
C604Y
N605H
N605K
N605S
N605Y
P607A
P607L
D608Y
P609G
P609H
P609L
P609S
M610I
M610L
M610T
V611I
R612*
R612G
R612L
R612P
R612Q
R612fs*1
G613D
G613V
A615V
V616A
V616L
R617Q
R617W
C618R
C618W
L619S
L619V
K621*
K621I
K621N
K621Q
Y622*
L623F
L623I
L623V
T624I
T624P
T624S
D625F
D625H
D625V
D625Y
D626E
D626G
D626N
D626Y
K627N
K627Q
K627R
L628I
L628P
L628R
L628V
S629C
S629F
S629P
S629Y
Q630*
Q630E
Q630H
Q630P
Q630R
Y631C
L632*
L632F
I633L
Q634*
Q634E
Q634H
V636A
V636L
V636R
Q637*
Q637K
Q637L
V638A
V638I
K640Q
K640R
Y641*
E642K
E642Q
Q643H
Q643L
Q643R
Y644*
Y644C
L645F
D646E
D646H
N647K
N647S
N647T
L648F
L648V
L649F
V650A
R651K
R651S
L653*
L653fs*2
L653fs*8
L654V
K655N
K656N
K656T
A657P
A657S
A657V
L658F
T659A
T659N
T659S
N660D
N660S
Q661E
Q661K
Q661fs*11
R662K
R662M
R662S
I663S
I663del
G664E
G664W
H665fs*7
F666C
F666L
F667L
W669*
W669C
W669G
W669R
W669fs*6
H670N
H670Q
H670R
L671F
L671I
L671V
L671fs*1
K672I
K672N
S673C
S673F
S673T
E674A
E674D
E674G
E674K
E674Q
E674V
M675I
M675T
M675V
H676R
H676Y
H676fs*24
N677H
N677K
N677S
K678*
K678E
K678T
T679R
V680A
V680D
V680I
V680fs*19
S681C
S681I
S681N
S681R
S681T
Q682*
Q682H
Q682R
R683K
R683M
R683S
R683T
F684Y
G685S
G685V
L687F
L687I
L688M
E689K
E689Q
S690F
S690Y
Y691C
C692*
C692R
R693C
R693G
R693H
R693L
R693P
R693S
A694T
A694V
G696R
G696W
M697fs*3
Y698*
Y698C
Y698H
L699F
L699V
H701Q
L702V
R704S
R704T
R704W
Q705H
Q705L
V706F
V706I
E707*
E707D
E707K
E707Q
A708T
M709I
M709V
E710K
K711E
K711N
K711Q
K711R
K711T
L712F
L712H
L712I
L712V
N714D
N714H
N714I
N714K
N714Y
L715V
T716A
T716I
T716N
T716S
D717E
D717H
D717N
D717V
D717_D725del
I718L
I718V
I718_L719del
L719F
L719I
L719R
L719V
K720E
K720I
Q721E
Q721H
Q721K
Q721P
E722D
E722K
E722Q
E722del
K723N
K723R
K723T
K724T
K724del
D725A
D725E
D725G
D725H
D725V
D725Y
D725_E726del
D725_K729del
D725_Q728 > L
D725_Q728del
D725_T727del
D725del
E726*
E726A
E726D
E726G
E726K
E726Q
E726V
T727A
T727K
T727R
T727S
T727del
Q728K
Q728R
K729N
V730A
V730I
V730L
Q731E
Q731H
Q731K
Q731R
M732I
K733N
K733T
F734I
F734L
F734V
L735I
L735S
L735V
V736A
V736F
V736I
V736L
E737*
E737D
E737K
E737Q
Q738E
Q738R
Q738fs*2
M739I
R741*
R741Q
P742A
P742R
P742S
P742T
D743E
D743G
D743H
D743N
D743V
F744I
F744L
F744V
M745I
M745L
M745T
D746A
D746G
D746H
D746N
D746V
D746Y
L748V
L748fs*5
Q749*
Q749H
Q749K
G750A
G750C
G750D
G750E
G750S
F751S
F751V
L752R
L752V
S753F
S753T
S753Y
P754A
P754S
P754T
P754del
L755I
L755Q
L755V
L755_N756 > P
N756K
N756S
N756Y
P757L
P757S
P757fs*5
A758S
A758T
A758V
H759D
H759N
H759Q
H759Y
Q760*
Q760E
Q760L
Q760R
L761R
G762E
G762R
N763T
L764F
L7641
L764P
L764V
R765G
R765S
L766P
L766fs*10
E767G
E767K
E767Q
E768*
E768D
E768K
E768Q
C769W
R770*
R770L
R770Q
I771L
I771N
I771S
I771T
M772I
M772V
S773F
S773P
S774C
S774F
A775V
K776R
R777K
R777S
R777fs*5
R777fs*22
P778S
L779V
W780S
L781F
L781S
L781W
N782D
W783*
W783S
E784D
N785I
N785S
P786A
P786Q
P786S
P786T
D787G
D787V
I788F
I788V
I788del
M789I
M789S
M789T
M789V
S790*
S790A
S790P
E791*
E791D
E791Q
L792*
L792F
L793V
L793fs*5
Q795*
Q795E
Q795H
Q795R
N796H
N796I
E798D
E798K
E798Q
I799L
I799M
I799V
I800F
I800M
F801L
F801N
F801V
N803H
N803Y
G804R
D805G
D805N
D805Y
D806G
D806Y
L807S
L807V
R808L
R808W
Q809*
Q809H
Q809K
D810H
D810Y
M811I
T813A
L814V
Q815*
Q815R
I816N
I816S
I816T
I816V
I817F
I817V
I817_I819del
I817fs*14
R818C
R818G
R818H
R818L
I819N
I819V
M820I
M820L
E821G
E821K
N822D
N822I
I823F
I823M
I823T
I823V
W824*
W824F
W824G
Q825*
Q827E
Q827K
G828D
L829F
L829I
L829P
D830G
D830N
D830V
L831F
L831I
L831V
R832*
R832L
R832Q
M833I
M833V
M833fs*1
L834S
L834V
P835A
P835L
P835T
Y836C
G837C
G837D
G837N
G837S
G837fs*30
C838F
C838Y
L839V
S840*
S840L
S840fs*27
I841V
I841fs*3
G842C
G842S
D843G
D843N
C844F
C844R
C844S
C844Y
V845L
V845W
V845_G846insCV
V845fs*4
G846*
L847F
L847H
L847R
L847V
I848L
I848M
I848T
E849*
E849D
E849K
E849Q
V850A
V850G
V850M
V851E
R852*
R852G
R852Q
N853K
N853S
S854A
S854C
S854F
S854P
H855L
H855R
H855S
H855T
H855Y
T856S
I857L
I857V
I860M
I860V
Q861*
Q861E
Q861H
Q861K
C862W
K863I
G864S
G865D
G865S
L866F
L866V
L866W
K8671
K867N
K867R
G868A
G868C
G868S
A869T
A869V
Q871L
F872L
F872V
N873D
N873H
N873K
N873S
S874N
H875N
H875R
H875Y
T876A
T8761
T876K
L8771
L877V
H878Y
Q879*
Q879H
Q879K
Q879L
Q879R
L8811
L881V
K882T
D883E
D883G
D883H
D883N
D883V
D883Y
K884*
K884N
N885S
K886E
K886N
K886R
G887A
G887E
G887R
E888D
E888G
E888K
E888Q
I889L
I889M
I889T
Y890C
Y890N
A892V
A893S
A893T
A893V
A893fs*3
I894L
I894V
D895E
D895H
D895N
D895Y
L896M
L896P
F897L
F897fs*18
T898I
T898P
T898fs*19
R899C
R899G
R899H
C901F
C901S
C901W
C901fs*19
A902P
A902T
A902V
G903*
G903A
G903E
Y904*
C905S
V906I
V906L
A907V
T908fs*15
F909C
F909L
I910L
I910V
L911F
L911M
L911fs*9
G912R
G914E
G914R
D915N
R916C
R916H
S919G
S919T
N920S
I921N
I921V
I921del
M922I
V923L
V923M
K924R
D925E
D926N
G927R
G927V
Q928R
L929F
L929M
F930S
H931N
H931Y
I932L
D933N
F934C
F934fs*23
H936R
F937fs*1
L938M
L938fs*19
D939G
D939N
H940R
H940Y
K941N
K941R
K942M
K942N
K943N
K944I
K944R
K944del
F945C
F945I
F945L
F945fs*4
F945fs*12
G946C
G946D
G946V
Y947*
Y947F
R949*
R949Q
E950*
E950D
E950Q
R951C
R951H
R951L
V952A
V952L
F954Y
F954fs*7
F954fs*11
V955F
L956F
T957I
T957P
Q958*
Q958H
Q958K
Q958L
Q958R
D959Y
F960L
L961F
I962M
I962T
V963M
I964N
I964fs*2
I964fs*22
S965N
S965T
K966I
K966R
G967A
G967E
G967R
A968S
A968T
A968V
Q969*
Q969H
Q969K
Q969R
E970*
E970A
E970G
E970K
E970Q
C971G
T972R
K973N
T974K
T974R
T974fs*3
R975K
R975T
E976*
E976D
E976G
E976K
E976Q
F977C
F977L
E978D
E978K
E978Q
R979M
R979S
F980V
Q981*
Q981E
Q981H
Q981K
Q981L
Q981R
E982D
E982K
E982Q
M983I
M983L
C984R
C984S
Y985C
Y985D
Y985H
Y985N
Y985S
K986fs*5
A987T
A987V
Y988H
Y988N
Y988S
L989Q
L989R
L989V
A990G
A990_I991 > V*
I991V
I991fs*26
R992*
R992Q
A995V
N996I
N996K
L997F
L997H
L997I
L997V
F998L
F998fs*14
I999M
I999R
I999V
N1000H
N1000K
N1000S
L1001F
L1001I
L1001V
L1001fs*4
L1001fs*17
F1002C
F1002I
F1002L
F1002V
S1003K
S1003L
S1003fs*15
M1004I
M1004L
M1004R
M1004V
M1004del
M1005I
M1005V
L1006F
L1006H
L1006R
G1007C
G1007D
G1007R
S1008C
G1009A
G1009E
G1009R
M1010I
M1010I
M1010V
P1011A
P1011L
P1011S
E1012D
E1012Q
Q1014K
Q1014R
S1015C
S1015F
S1015Y
F1016I
F1016L
F1016S
F1016V
F1016Y
D1017G
D1017H
D1017V
D1017Y
D1018N
I1019M
I1019S
I1019T
A1020K
A1020S
A1020T
A1020V
Y1021C
Y1021F
Y1021H
Y1021N
Y1021S
I1022M
I1022T
I1022_R1023insFLYVCTIAYI
R1023*
R1023L
R1023P
R1023Q
K1024N
K1024T
T1025A
T1025I
T1025N
T1025S
L1026P
A1027T
A1027V
L1028F
L1028I
L1028S
L1028V
D1029E
D1029G
D1029H
D1029N
D1029Y
K1030*
K1030E
K1030R
T1031A
T1031N
T1031P
E1032*
E1032A
E1032D
E1032K
Q1033E
Q1033K
Q1033P
Q1033R
E1034G
E1034K
E1034Q
A1035T
A1035V
L1036F
L1036K
L1036S
E1037D
E1037K
E1037Q
Y1038C
Y1038F
Y1038H
Y1038N
Y1038S
F1039I
F1039S
M1040I
M1040K
M1040L
K1041*
Q1042R
Q1042fs*25
1043_1044MN > IY
M1043I
M1043L
M1043T
M1043V
M1043_N1044 > IK
M1043_N1044 > IR
N1044D
N1044H
N1044I
N1044K
N1044R
N1044S
N1044T
N1044Y
D1045A
D1045G
D1045H
D1045N
D1045V
D1045Y
A1046T
H1047A
H1047D
H1047I
H1047L
H1047N
H1047Q
H1047R
H1047Y
H1047_H1048 > RR
H1048L
H1048N
H1048R
G1049A
G1049C
G1049D
G1049R
G1049S
G1049W
G1050A
G1050D
G1050S
W1051C
W1051L
W1051fs*16
T1052A
T1052I
T1052K
T1052R
T1053fs*15
K1054T
K1054_M1055 > NGLDLPHN*TACIEM
M1055I
M1055L
M1055V
D1056H
D1056N
W1057*
W1057C
I1058F
I1058L
I1058M
H1060L
T1061I
T1061K
I1062L
I1062V
K1063*
Q1064H
Q1064_H1065insQWTTKMDWIFHTIKQ
Q1064fs*6
Q1064fs*7
Q1064fs*9
Q1064fs*24
Q1064fs*5+
H1065L
H1065Q
H1065R
H1065Y
H1065_*1069 > ?
H1065_A1066 > LKLK
H1065fs*?
H1065fs*4
H1065fs*5
H1065fs*8
H1065fs*8
H1065fs*1+
H1065fs*15
H1065fs*6+
A1066_*1069 > ?
A1066_L1067insCV
A1066fs*2+
A1066fs*4
A1066fs*5
A1066fs*5+
A1066fs*8
A1066I
A1066L
A1066L
L1067F
L1067V
L1067W
L1067_*1069 > FKLKKN*
L1067fs*4
L1067fs*5
L1067fs*6
L1067fs*7
L1067fs*7
L1067fs*11+
L1067fs*20
L1067fs*5+
N1068K
N1068fs*1
N1068fs*3
N1068fs*4
N1068fs*5
N1068fs*5
N1068fs*7
N1068fs*1+
N1068fs*10
N1068fs*11
N1068fs*21

As used herein, the term “PI3Kα-mediated” disorders, diseases, and/or conditions means any disease or other deleterious condition in which PI3Kα or a mutant thereof is known to play a role. Accordingly, another embodiment of the present disclosure relates to treating or lessening the severity of one or more diseases in which PI3Kα, or a mutant thereof, is known to play a role. Such PI3Kα-mediated disorders include, but are not limited to, cellular proliferative disorders (e.g., cancer). In some embodiments, the PI3Kα-mediated disorder is a disorder mediated by a mutant PI3Kα. In some embodiments, the PI3Kα-mediated disorder is a disorder mediated by a PI3Kα containing at least one of the following mutations: H1047R, E542K, and E545K.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof, a therapeutically effective amount of a compound of the present disclosure, or a pharmaceutically acceptable composition thereof.

In some embodiments, the method of treatment comprises the steps of: (i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

In some embodiments, the method of treatment comprises the steps of: (i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant PI3Kα. In some embodiments, the subject has PI3Kα containing at least one of the following mutations: H1047R, E542K, and E545K.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric; glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; chronic myelogenous leukemia; lymphocytic leukemia; myeloid leukemia; brain; a carcinoma of the brain; oral cavity and pharynx; larynx; small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma; multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cancer is ovarian cancer. In some embodiments, the ovarian cancer is clear cell ovarian cancer.

In some embodiments, the cellular proliferative disease has mutant PI3Kα. In some embodiments, the cancer has mutant PI3Kα. In some embodiments, the breast cancer has mutant PI3Kα. In some embodiments, the ovarian cancer has mutant PI3Kα.

In some embodiments, the cellular proliferative disease has PI3Kα containing at least one of the following mutations: H1047R, E542K, and E545K. In some embodiments, the cancer has PI3Kα containing at least one of the following mutations: H1047R, E542K, and E545K. In some embodiments, the breast cancer has PI3Kα containing at least one of the following mutations: H1047R, E542K, and E545K. In some embodiments, the ovarian cancer has PI3Kα containing at least one of the following mutations: H1047R, E542K, and E545K.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma. In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of PI3Kα, somatic mutation of PIK3CA, germline mutations or somatic mutation of PTEN, or mutations and translocation of p85α that serve to up-regulate the p85-p110 complex. In some embodiments, the cellular proliferative disease displays overexpression or amplification of PI3Kα. In some embodiments, the cellular proliferative disease displays somatic mutation of PIK3CA. In some embodiments, the cellular proliferative disease displays germline mutations or somatic mutation of PTEN. In some embodiments, the cellular proliferative disease displays mutations and translocation of p85α that serve to up-regulate the p85-p110 complex.

### Additional Disorders

In some embodiments, the PI3Kα-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g., haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the PI3Kα-mediated disorder is polycythemia vera, essential thrombocythemia, or myelofibrosis with myeloid metaplasia. In some embodiments, the PI3Kα-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the PI3Kα-mediated disorder is polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, or scleroderma. In some embodiments, the PI3Kα-mediated disorder is vitiligo, hypersensitivity angiitis, urticaria, bullous pemphigoid, lupus erythematosus, pemphisus, epidermolysis bullosa acquisita, or autoimmune haematogical disorders (e.g., haemolytic anaemia, aplastic anaemia, pure red cell anaemia and idiopathic thrombocytopenia). In some embodiments, the PI3Kα-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g., ulcerative colitis and Crohn's disease).

In some embodiments, the PI3Kα-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the PI3Kα-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the PI3Kα-mediated disorder is retinopathy, such as diabetic retinopathy or hyperbaric oxygen-induced retinopathy, and conditions characterized by elevated intraocular pressure or secretion of ocular aqueous humor, such as glaucoma.

### Routes of Administration and Dosage Forms

The compounds and compositions, according to the methods of the present disclosure, may be administered using any amount and any route of administration effective for treating or lessening the severity of the disorder (e.g., a proliferative disorder). The e^{YA}ct amount required will vary from subject to subject, depending on the species, age, and general condition of the subject, the severity of the infection, the particular agent, its mode of administration, and the like. Compounds of the disclosure are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds of the disclosure may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this disclosure with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years, or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the disclosure relates to a method of inhibiting protein kinase activity in a biological sample comprising the step of contacting said biological sample with a compound of this disclosure, or a composition comprising said compound. According to another embodiment, the disclosure relates to a method of inhibiting activity of a PI3K, or a mutant thereof, in a biological sample comprising the step of contacting said biological sample with a compound of this disclosure, or a composition comprising said compound. According to another embodiment, the disclosure relates to a method of inhibiting activity of PI3Kα, or a mutant thereof, in a biological sample comprising the step of contacting said biological sample with a compound of this disclosure, or a composition comprising said compound. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting PI3Kα over one or both of PI3Kδ and PI3Kγ. In some embodiments, a compound of the present disclosure is more than 5-fold selective over PI3Kδ and PI3Kγ. In some embodiments, a compound of the present disclosure is more than 10-fold selective over PI3Kδ and PI3Kγ. In some embodiments, a compound of the present disclosure is more than 50-fold selective over PI3Kδ and PI3Kγ. In some embodiments, a compound of the present disclosure is more than 100-fold selective over PI3Kδ and PI3Kγ. In some embodiments, a compound of the present disclosure is more than 200-fold selective over PI3Kδ and PI3Kγ. In some embodiments, the PI3Kα is a mutant PI3Kα. In some embodiments, the PI3Kα contains at least one of the following mutations: H1047R, E542K, and E545K.

In another embodiment, the disclosure provides a method of selectively inhibiting a mutant PI3Kα over a wild-type PI3Kα. In some embodiments, a compound of the present disclosure is more than 5-fold selective for mutant PI3Kα over wild-type PI3Kα. In some embodiments, a compound of the present disclosure is more than 10-fold selective for mutant PI3Kα over wild-type PI3Kα. In some embodiments, a compound of the present disclosure is more than 50-fold selective for mutant PI3Kα over wild-type PI3Kα. In some embodiments, a compound of the present disclosure is more than 100-fold selective for mutant PI3Kα over wild-type PI3Kα. In some embodiments, a compound of the present disclosure is more than 200-fold selective for mutant PI3Kα over wild-type PI3Kα. In some embodiments, the mutant PI3Kα contains at least one of the following mutations: H1047R, E542K, and E545K.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of activity of a PI3K (for example, PI3Kα, or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound of the present disclosure, or a composition comprising said compound.

According to another embodiment, the disclosure relates to a method of inhibiting activity of a PI3K, or a mutant thereof, in a patient comprising the step of administering to said patient a compound of the present disclosure, or a composition comprising said compound. In some embodiments, the disclosure relates to a method of inhibiting activity of PI3Kα, or a mutant thereof, in a patient comprising the step of administering to said patient a compound of the present disclosure, or a composition comprising said compound. In some embodiments, the PI3Kα is a mutant PI3Kα. In some embodiments, the PI3Kα contains at least one of the following mutations: H1047R, E542K, and E545K.

According to another embodiment, the present disclosure provides a method for treating a disorder mediated by a PI3K, or a mutant thereof, in a patient in need thereof, comprising the step of administering to said patient a compound according to the present disclosure or pharmaceutically acceptable composition thereof. In some embodiments, the present disclosure provides a method for treating a disorder mediated by PI3Kα, or a mutant thereof, in a patient in need thereof, comprising the step of administering to said patient a compound according to the present disclosure or pharmaceutically acceptable composition thereof. In some embodiments, the PI3Kα is a mutant PI3Kα. In some embodiments, the PI3Kα contains at least one of the following mutations: H1047R, E542K, and E545K.

According to another embodiment, the present disclosure provides a method of inhibiting signaling activity of PI3Kα, or a mutant thereof, in a subject, comprising administering a therapeutically effective amount of a compound according to the present disclosure, or a pharmaceutically acceptable composition thereof, to a subject in need thereof. In some embodiments, the present disclosure provides a method of inhibiting PI3Kα signaling activity in a subject, comprising administering a therapeutically effective amount of a compound according to the present disclosure, or a pharmaceutically acceptable composition thereof, to a subject in need thereof. In some embodiments, the PI3Kα is a mutant PI3Kα. In some embodiments, the PI3Kα contains at least one of the following mutations: H1047R, E542K, and E545K. In some embodiments, the subject has a mutant PI3Kα. In some embodiments, the subject has PI3Kα containing at least one of the following mutations: H1047R, E542K, and E545K.

The compounds described herein can also inhibit PI3Kα function through incorporation into agents that catalyze the destruction of PI3Kα. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of PI3Kα to the E3 ligase will thus result in the destruction of the PI3Kα protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions of this disclosure. As used herein, additional therapeutic agents that are normally administered to treat a particular disease, or condition, are known as “appropriate for the disease, or condition, being treated.”

Additionally, PI3K serves as a second messenger node that integrates parallel signaling pathways, and evidence is emerging that the combination of a PI3K inhibitor with inhibitors of other pathways will be useful in treating cancer and cellular proliferative diseases.

Accordingly, in certain embodiments, the method of treatment comprises administering the compound or composition of the disclosure in combination with one or more additional therapeutic agents. In certain other embodiments, the methods of treatment comprise administering the compound or composition of the disclosure as the only therapeutic agent.

Approximately 20-30% of human breast cancers overexpress Her-2/neu-ErbB2, the target for the drug trastuzumab. Although trastuzumab has demonstrated durable responses in some patients expressing Her2/neu-ErbB2, only a subset of these patients respond. Recent work has indicated that this limited response rate can be substantially improved by the combination of trastuzumab with inhibitors of PI3K or the PI13K/AKT pathway (Chan et al., Breast Can. Res. Treat. 91:187 (2005), Woods Ignatoski et al., Brit. J. Cancer 82:666 (2000), Nagata et al., Cancer Cell 6:117 (2004)). Accordingly, in certain embodiments, the method of treatment comprises administering the compound or composition of the disclosure in combination with trastuzumab. In certain embodiments, the cancer is a human breast cancer that overexpresses Her-2/neu-ErbB2.

A variety of human malignancies express activating mutations or increased levels of Her1/EGFR and a number of antibody and small molecule inhibitors have been developed against this receptor tyrosine kinase including tarceva, gefitinib and erbitux. However, while EGFR inhibitors demonstrate anti-tumor activity in certain human tumors (e.g., NSCLC), they fail to increase overall patient survival in all patients with EGFR-expressing tumors. This may be rationalized by the fact that many downstream targets of Her1/EGFR are mutated or deregulated at high frequencies in a variety of malignancies, including the PI3K/Akt pathway.

For example, gefitinib inhibits the growth of an adenocarcinoma cell line in in vitro assays. Nonetheless, sub-clones of these cell lines can be selected that are resistant to gefitinib that demonstrate increased activation of the PI3/Akt pathway. Down-regulation or inhibition of this pathway renders the resistant sub-clones sensitive to gefitinib (Kokubo et al., Brit. J. Cancer 92:1711 (2005)). Furthermore, in an in vitro model of breast cancer with a cell line that harbors a PTEN mutation and over-expresses EGFR inhibition of both the PI3K/Akt pathway and EGFR produced a synergistic effect (She et al., Cancer Cell 8:287-297 (2005)). These results indicate that the combination of gefitinib and PI3K/Akt pathway inhibitors would be an attractive therapeutic strategy in cancer.

Accordingly, in certain embodiments, the method of treatment comprises administering the compound or composition of the disclosure in combination with an inhibitor of Her1/EGFR. In certain embodiments, the method of treatment comprises administering the compound or composition of the disclosure in combination with one or more of tarceva, gefitinib, and erbitux. In certain embodiments, the method of treatment comprises administering the compound or composition of the disclosure in combination with gefitinib. In certain embodiments, the cancer expresses activating mutations or increased levels of Her1/EGFR.

The combination of AEE778 (an inhibitor of Her-2/neu/ErbB2, VEGFR and EGFR) and RAD001 (an inhibitor of mTOR, a downstream target of Akt) produced greater combined efficacy that either agent alone in a glioblastoma xenograft model (Goudar et al., Mol. Cancer. Ther. 4:101-112 (2005)).

Anti-estrogens, such as tamoxifen, inhibit breast cancer growth through induction of cell cycle arrest that requires the action of the cell cycle inhibitor p27Kip. Recently, it has been shown that activation of the Ras-Raf-MAP Kinase pathway alters the phosphorylation status of p27Kip such that its inhibitory activity in arresting the cell cycle is attenuated, thereby contributing to anti-estrogen resistance (Donovan, et al, J. Biol. Chem. 276:40888, (2001)). As reported by Donovan et al., inhibition of MAPK signaling through treatment with MEK inhibitor reversed the aberrant phosphorylation status of p27 in hormone refractory breast cancer cell lines and in so doing restored hormone sensitivity. Similarly, phosphorylation of p27Kip by Aid also abrogates its role to arrest the cell cycle (Viglietto et al., Nat. Med. 8:1145 (2002)).

Accordingly, in certain embodiments, the method of treatment comprises administering the compound or composition of the disclosure in combination with a treatment for a hormone-dependent cancer. In certain embodiments, the method of treatment comprises administering the compound or composition of the disclosure in combination with tamoxifen. In certain embodiments, the cancer is a hormone dependent cancer, such as breast and prostate cancers. By this use, it is aimed to reverse hormone resistance commonly seen in these cancers with conventional anticancer agents.

In hematological cancers, such as chronic myelogenous leukemia (CML), chromosomal translocation is responsible for the constitutively activated BCR-Ab1 tyrosine kinase. The afflicted patients are responsive to imatinib, a small molecule tyrosine kinase inhibitor, as a result of inhibition of Ab1 kinase activity. However, many patients with advanced stage disease respond to imatinib initially, but then relapse later due to resistance-conferring mutations in the Ab1 kinase domain. In vitro studies have demonstrated that BCR-Ab1 employs the Ras-Raf kinase pathway to elicit its effects. In addition, inhibiting more than one kinase in the same pathway provides additional protection against resistance-conferring mutations.

Accordingly, in another aspect, the compounds and compositions of the disclosure are used in combination with at least one additional agent selected from the group of kinase inhibitors, such as imatinib, in the treatment of hematological cancers, such as chronic myelogenous leukemia (CML). By this use, it is aimed to reverse or prevent resistance to said at least one additional agent.

Because activation of the PI3K/Akt pathway drives cell survival, inhibition of the pathway in combination with therapies that drive apoptosis in cancer cells, including radiotherapy and chemotherapy, will result in improved responses (Ghobrial et al., CA Cancer J. Clin 55:178-194 (2005)). As an example, combination of PI3 kinase inhibitor with carboplatin demonstrated synergistic effects in both in vitro proliferation and apoptosis assays as well as in in vivo tumor efficacy in a xenograft model of ovarian cancer (Westfall and Skinner, Mol. Cancer Ther. 4:1764-1771 (2005)).

In some embodiments, the one or more additional therapeutic agents is selected from antibodies, antibody-drug conjugates, kinase inhibitors, immunomodulators, and histone deacetylase inhibitors. Synergistic combinations with PIK3CA inhibitors and other therapeutic agents are described in, for example, Castel et al., Mol. Cell Oncol. (2014)1(3) e963447.

In some embodiments, the one or more additional therapeutic agent is selected from the following agents, or a pharmaceutically acceptable salt thereof: BCR-ABL inhibitors (see, e.g., Ultimo et al. Oncotarget (2017) 8 (14) 23213-23227.): e.g., imatinib, inilotinib, nilotinib, dasatinib, bosutinib, ponatinib, bafetinib, danusertib, saracatinib, PF03814735; ALK inhibitors (see, e.g., Yang et al. Tumour Biol. (2014) 35 (10) 9759-67): e.g., crizotinib, NVP-TAE684, ceritinib, alectinib, brigatinib, entrecinib, lorlatinib; BRAF inhibitors (see, e.g., Silva et al. Mol. Cancer Res. (2014) 12, 447-463): e.g., vemurafenib, dabrafenib; FGFR inhibitors (see, e.g., Packer et al. Mol. Cancer Ther. (2017) 16(4) 637-648): e.g., infigratinib, dovitinib, erdafitinib, TAS-120, pemigatinib, BLU-554, AZD4547; FLT3 inhibitors: e.g., sunitinib, midostaurin, tanutinib, sorafenib, lestaurtinib, quizartinib, and crenolanib; MEK Inhibitors (see, e.g., Jokinen et al. Ther. Adv. Med. Oncol. (2015) 7(3) 170-180): e.g., trametinib, cobimetinib, binimetinib, selumetinib; ERK inhibitors: e.g., ulixertinib, MK 8353, LY 3214996; KRAS inhibitors: e.g., AMG-510, MRTX849, ARS-3248; Tyrosine kinase inhibitors (see, e.g., Makhov et al. Mol. Cancer. Ther. (2012) 11(7) 1510-1517): e.g., erlotinib, linifanib, sunitinib, pazopanib; Epidermal growth factor receptor (EGFR) inhibitors (see, e.g., She et al. BMC Cancer (2016) 16, 587): gefitnib, osimertinib, cetuximab, panitumumab; HER2 receptor inhibitors (see, e.g., Lopez et al. Mol. Cancer Ther. (2015) 14(11) 2519-2526): e.g., trastuzumab, pertuzumab, neratinib, lapatinib, lapatinib; MET inhibitors (see, e.g., Hervieu et al. Front. Mol. Biosci. (2018) 5, 86): e.g., crizotinib, cabozantinib; CD20 antibodies: e.g., rituximab, tositumomab, ofatumumab; DNA Synthesis inhibitors: e.g., capecitabine, gemcitabine, nelarabine, hydroxycarbamide; Antineoplastic agents (see, e.g., Wang et al. Cell Death & Disease (2018) 9, 739): e.g., oxaliplatin, carboplatin, cisplatin; Immunomodulators: e.g., afutuzumab, lenalidomide, thalidomide, pomalidomide; CD40 inhibitors: e.g., dacetuzumab; Pro-apoptotic receptor agonists (PARAs): e.g., dulanermin; Heat Shock Protein (HSP) inhibitors (see, e.g., Chen et al. Oncotarget (2014) 5 (9). 2372-2389): e.g., tanespimycin; Hedgehog antagonists (see, e.g., Chaturvedi et al. Oncotarget (2018) 9 (24), 16619-16633): e.g., vismodegib; Proteasome inhibitors (see, e.g., Lin et al. Int. J. Oncol. (2014) 44 (2), 557-562): e.g., bortezomib; PI3K inhibitors: e.g., pictilisib, dactolisib, alpelisib, buparlisib, taselisib, idelalisib, duvelisib, umbralisib; SHP2 inhibitors (see, e.g., Sun et al. Am. J. Cancer Res. (2019) 9 (1), 149-159: e.g., SHP099, RMC-4550, RMC-4630); BCL-2 inhibitors (see, e.g., Bojarczuk et al. Blood (2018) 133 (1), 70-80): e.g., venetoclax; Aromatase inhibitors (see, e.g., Mayer et al. Clin. Cancer Res. (2019) 25 (10), 2975-2987): exemestane, letrozole, anastrozole, fulvestrant, tamoxifen; mTOR inhibitors (see, e.g., Woo et al. Oncogenesis (2017) 6, e385): e.g., temsirolimus, ridaforolimus, everolimus, sirolimus; CTLA-4 inhibitors (see, e.g., O'Donnell et al. (2018) 48, 91-103): e.g., tremelimumab, ipilimumab; PD1 inhibitors (see O'Donnell, supra): e.g., nivolumab, pembrolizumab; an immunoadhesin; Other immune checkpoint inhibitors (see, e.g., Zappasodi et al. Cancer Cell (2018) 33, 581-598, where the term “immune checkpoint” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta): e.g., pidilizumab, AMP-224; PDL1 inhibitors (see e.g., O'Donnell supra): e.g., MSB0010718C; YW243.55.570, MPDL3280A; MEDI-4736, MSB-0010718C, or MDX-1105; Histone deacetylase inhibitors (HDI, see, e.g., Rahmani et al. Clin. Cancer Res. (2014) 20(18), 4849-4860): e.g. vorinostat; Androgen Receptor inhibitors (see e.g., Thomas et al. Mol. Cancer Ther. (2013) 12(11), 2342-2355): e.g., enzalutamide, abiraterone acetate, orteronel, galeterone, seviteronel, bicalutamide, flutamide; Androgens: e.g., fluoxymesterone; CDK4/6 inhibitors (see, e.g., Gul et al. Am. J. Cancer Res. (2018) 8(12), 2359-2376): e.g., alvocidib, palbociclib, ribociclib, trilaciclib, abemaciclib.

In some embodiments, the one or more additional therapeutic agent is selected from the following agents: anti-FGFR antibodies; FGFR inhibitors, cytotoxic agents; Estrogen Receptor-targeted or other endocrine therapies, immune-checkpoint inhibitors, CDK inhibitors, Receptor Tyrosine Kinase inhibitors, BRAF inhibitors, MEK inhibitors, other PI3K inhibitors, SHP2 inhibitors, and SRC inhibitors. (See Katoh, Nat. Rev. Clin. Oncol. (2019), 16:105-122; Chae, et al. Oncotarget (2017), 8:16052-16074; Formisano et al., Nat. Comm. (2019), 10:1373-1386; and references cited therein.)

The structure of the active compounds identified by code numbers, generic or trade names may be taken from the actual edition of the standard compendium The Merck Index or from databases, e.g., Patents International (e.g., IMS World Publications).

A compound of the current disclosure may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current disclosure can be administered alone or in combination with one or more other therapeutic compounds, possible combination therapy taking the form of fixed combinations or the administration of a compound of the disclosure and one or more other therapeutic compounds being staggered or given independently of one another, or the combined administration of fixed combinations and one or more other therapeutic compounds. A compound of the current disclosure can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this disclosure in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of the current disclosure, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. In some embodiments, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In such compositions, a dosage of between 0.01-1,000 μg/kg body weight/day of the additional therapeutic agent can be administered.

The amount of additional therapeutic agent present in the compositions of this disclosure will be no more than the amount that would normally be administered in a composition comprising that therapeutic agent as the only active agent. In some embodiments, the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this disclosure are another embodiment of the present disclosure.

Any of the compounds and/or compositions of the disclosure may be provided in a kit comprising the compounds and/or compositions. Thus, in some embodiments, the compound and/or composition of the disclosure is provided in a kit.

The disclosure is further described by the following non-limiting Examples.

### EXAMPLES

Examples are provided herein to facilitate a more complete understanding of the disclosure. The following examples serve to illustrate the exemplary modes of making and practicing the subject matter of the disclosure. However, the scope of the disclosure is not to be construed as limited to specific embodiments disclosed in these examples, which are illustrative only.

As depicted in the Examples and General Schemes below, in certain exemplary embodiments, compounds are prepared according to the following general procedures. It will be appreciated that, although the general methods depict the synthesis of certain compounds of the present disclosure, the following general methods, and other methods known to one of ordinary skill in the art, can be applied to other classes and subclasses and species of each of these compounds, as described herein. Additional compounds of the disclosure were prepared by methods substantially similar to those described herein in the Examples and methods known to one skilled in the art.

In the description of the synthetic methods described below, unless otherwise stated, it is to be understood that all reaction conditions (for example, reaction solvent, atmosphere, temperature, duration, and workup procedures) are selected from the standard conditions for that reaction, unless otherwise indicated. The starting materials for the Examples are either commercially available or are readily prepared by standard methods from known materials.

### List of Abbreviations

aq: aqueous
Ac: acetyl
ACN or MeCN: acetonitrile
AmF: ammonium formate
anhyd.: anhydrous
BINAP: (±)-2,2′-Bis(diphenylphosphino)-1,1′-binaphthalene
Bn: Benzyl
conc.: concentrated
DBU: 1,8-Diazabicyclo[5.4.0]undec-7-ene
DCE: Dichloroethane
DCM: Dichloromethane
DIPEA: Diisopropylamine
DMF: N,N-dimethylformamide
DMP: Dess-Martin periodinane
DMPU: N,N′-Dimethylpropyleneurea
DMSO: dimethylsulfoxide
DIPEA: diisopropylethylamine
EA or EtOAc: ethyl acetate
EDCI, EDC, or EDAC: 1-Ethyl-3-(3-dimethylaminopropyl)carbodiimide
equiv or eq: molar equivalents
Et: ethyl
HATU: 1-[Bis(dimethylamino)methylene]-1H-1,2,3-triazolo[4,5-b]pyridinium 3-oxid He^{YA}fluorophosphate
HPLC: high pressure liquid chromatography
LCMS or LC-MS: liquid chromatography-mass spectrometry
Ms: methanesulfonyl
NBS: N-bromosuccinimide
NMR: nuclear magnetic resonance
PE: petroleum ether
PMB: p-methoxybenzyl
rt or RT: room temperature
sat: saturated
TBS: tert-butyldimethylsilyl
TEA: triethylamine
Tf: trifluoromethanesulfonate
TFA: trifluoroacetic acid
THF: tetrahydrofuran
TLC: thin layer chromatography
Tol: toluene
UV: ultra violet

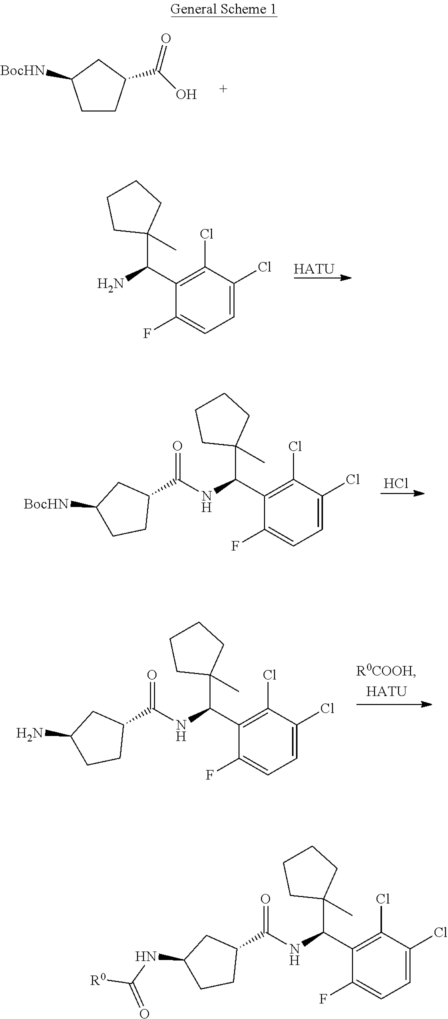

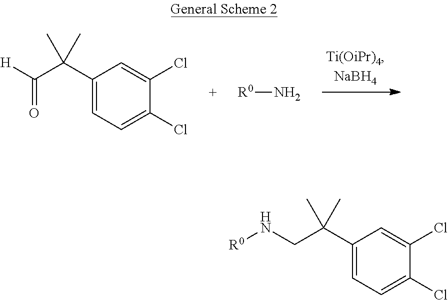

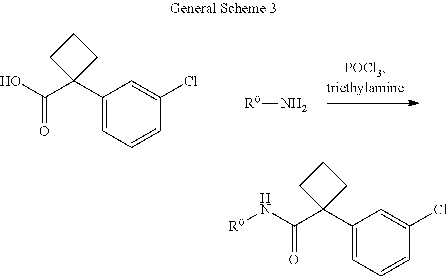

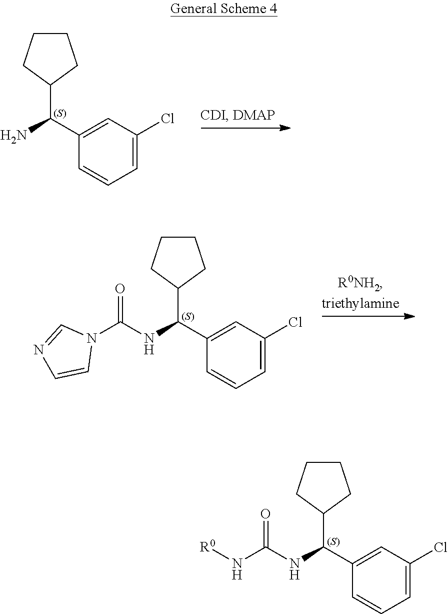

In some examples, compounds of the present disclosure were synthesized in accordance with the exemplary procedures shown in General Schemes 1, 2, 3, or 4. For the purposes of these schemes, R⁰ is an illustrative variable which, when taken together with its contiguous atoms in each instance, represents a commercially available compound, a compound disclosed herein, or other starting materials readily ascertained by one of skill in the art that result in the compounds of the present dislosure. It will be appreciated by one of skill in the art that certain reagents depicted in the General Schemes can be substituted with an appropriate reagent to accomplish an equivalent or substantially similar reaction.

### LC-MS and GC-MS Methods:

Method A: The analytical LC-MS system is equipped with Shimadzu LCMS-2020, PDA detector (operating at 254 nm), ELSD detector, and ESI-source operating in positive ion mode. LC conditions: the column is HALO C18 30*3.0 mm, 2 μm, operating at 40° C. with 1.5 mL/min of a binary gradient consisting of water+0.1% formic acid (A) and acetonitrile+0.1% formic acid (B). The retention time is expressed in minutes based on UV-trace at 254 nm.

| | | | |
|---|---|---|---|
| Gradient: | 0.01 | min | 5% B |
| | 1.00 | min | 100% B |
| | 1.40 | min | 100% B |
| | 1.42 | min | 5% B |
| Total run time: | 1.5 | min | |

Method B: The analytical LC-MS system is equipped with Shimadzu LCMS-2020, PDA detector (operating at 254 nm), ELSD detector, and ESI-source operating in positive ion mode. LC conditions: the column is Shim-pack Scepter C18-120, 33*3.0 mm, 3 μm, operating at 30° C. with 1.5 mL/min of a binary gradient consisting of water+5 mM NH₄HCO₃ (A) and acetonitrile (B). The retention time is expressed in minutes based on UV-trace at 254 nm.

| | | | |
|---|---|---|---|
| Gradient: | 0.01 | min | 10% B |
| | 1.20 | min | 95% B |
| | 1.80 | min | 95% B |
| | 1.82 | min | 10% B |
| Total run time: | 2.0 | min | |

Method C: The analytical LC-MS system is equipped with Shimadzu LCMS-2020, PDA detector (operating at 254 nm), ELSD detector, and ESI-source operating in positive ion mode. LC conditions: the column is HALO C18 30*3.0 mm, 2 μm, operating at 40° C. with 1.5 mL/min of a binary gradient consisting of water+0.05% trifluoroacetic acid (A) and acetonitrile+0.05% trifluoroacetic acid (B). The retention time is expressed in minutes based on UV-trace at 254 nm.

| | | | |
|---|---|---|---|
| Gradient: | 0.01 | min | 5% B |
| | 1.20 | min | 100% B |
| | 1.80 | min | 100% B |
| | 1.82 | min | 5% B |
| Total run time: | 2.0 | min | |

Method D: The analytical LC-MS system is equipped with Shimadzu LCMS-2020, PDA detector (operating at 254 nm), ELSD detector, and ESI-source operating in positive ion mode. LC conditions: the column is Shim-pack ScepterC18-120, 33*3.0 mm, 3 μm, operating at 30° C. with 1.5 mL/min of a binary gradient consisting of water+6.5 mM NH₄HCO₃+ammonia (pH=10) (A) and acetonitrile (B). The retention time is expressed in minutes based on UV-trace at 254 nm.

| | | | |
|---|---|---|---|
| Gradient: | 0.01 | min | 10% B |
| | 1.20 | min | 95% B |
| | 1.80 | min | 95% B |
| | 1.82 | min | 10% B |
| Total run time: | 2.0 | min | |

Method E: The analytical LC-MS system is equipped with Shimadzu LCMS-2020, PDA detector (operating at 254 nm), ELSD detector, and ESI-source operating in positive ion mode. LC conditions: the column is Kinetex EVO C18 50*3.0 mm, 2.6 μm, operating at 40° C. with 1.2 mL/min of a binary gradient consisting of water+0.04% NH₄OH (A) and acetonitrile (B). The retention time is expressed in minutes based on UV-trace at 254 nm.

| | | | |
|---|---|---|---|
| Gradient: | 0.01 | min | 10% B |
| | 1.20 | min | 95% B |
| | 1.80 | min | 95% B |
| | 1.82 | min | 10% B |
| Total run time: | 2.0 | min | |

Method F: The analytical LC-MS system is equipped with Shimadzu LCMS-2020, PDA detector (operating at 254 nm), ELSD detector, and ESI-source operating in positive ion mode. LC conditions: the column is Kinetex EVO C18 50*3.0 mm, 2.6 μm, operating at 40° C. with 1.2 mL/min of a binary gradient consisting of water+0.04% NH₄OH (A) and acetonitrile (B). The retention time is expressed in minutes based on UV-trace at 254 nm.

| | | |
|---|---|---|
| Gradient: | 0.01 min | 10% B |
| | 2.00 min | 95% B |
| | 2.60 min | 95% B |
| | 2.70 min | 10% B |
| Total run time: | 2.80 min | |

Method G: The analytical LC-MS system is equipped with Shimadzu LCMS-2020, PDA detector (operating at 254 nm), ELSD detector, and ESI-source operating in positive ion mode. LC conditions: the column is Poroshell HPH-C18 50*3.0 mm, 4 μm, operating at 40° C. with 1.5 mL/min of a binary gradient consisting of water+5 mM NH₄HCO₃ (A) and acetonitrile (B). The retention time is expressed in minutes based on UV-trace at 254 nm.

| | | |
|---|---|---|
| Gradient: | 0.01 min | 10% B |
| | 1.20 min | 95% B |
| | 1.80 min | 95% B |
| | 1.85 min | 10% B |
| Total run time: | 2.0 min | |

Method H: The analytical LC-MS system is equipped with Shimadzu LCMS-2020, PDA detector (operating at 254 nm), ELSD detector, and ESI-source operating in positive ion mode. LC conditions: the column is Shim-Pack-Scepter C18 33*3.0 mm, 3.0 μm, operating at 40° C. with 1.2 mL/min of a binary gradient consisting of water+0.1% Formic acid (A) and acetonitrile+0.07% Formic acid (B). The retention time is expressed in minutes based on UV-trace at 254 nm.

| | | |
|---|---|---|
| Gradient: | 0.01 min | 5% B |
| | 1.30 min | 95% B |
| | 1.75 min | 95% B |
| | 1.80 min | 5% B |
| Total run time: | 1.85 min | |

Method I: The analytical LC-MS system is equipped with Shimadzu LCMS-2020, PDA detector (operating at 220/254 nm), and ESI-source operating in positive ion mode. LC conditions: the column is Kinetex EVO C18 30*2.1 mm, 5 μm, operating at 50° C. with 1.5 mL/min of a binary gradient consisting of water+0.0375% TFA (A) and acetonitrile+0.01875% TFA (B). The retention times (t_{R}) are expressed in minutes based on UV-trace at 254 nm.

| | | |
|---|---|---|
| Gradient: | 0.01 min | 5% B |
| | 0.80 min | 95% B |
| | 1.20 min | 95% B |
| | 1.21 min | 5% B |
| | 1.55 min | 5% B |
| Total run time: | 1.55 min | |

Method J: The analytical LC-MS system is equipped with Shimadzu LCMS-2020, PDA detector (operating at 220/254 nm), and ESI-source operating in positive ion mode. LC conditions: the column is HALO C18 30*3.0 mm, 5 μm, operating at 50° C. with 2.0 mL/min of a binary gradient consisting of water+0.0375% TFA (A) and acetonitrile+0.01875% TFA (B). The retention times (t_{R}) are expressed in minutes based on UV-trace at 254 nm.

| | | |
|---|---|---|
| Gradient: | 0.01 min | 5% B |
| | 0.40 min | 95% B |
| | 0.75 min | 95% B |
| | 0.76 min | 5% B |
| | 1.05 min | 5% B |
| Total run time: | 1.05 min | |

Method K: Column: Waters Acquity UPLC CSH C18, 1.8 μm, 2.1×30 mm at 40° C.; Gradient: 5% to 100% B in 2.0 minutes; hold 100% B for 0.7 minute; run time: 2.7 min; flow 0.9 mL/min; Eluents: A=Milli-Q H₂O+10 mM ammonium formate; pH: 3.8; Eluent B: acetonitrile (no additive); Waters UPLC system equipped with: UV Detector=Waters Acquity PDA (198-360 nm), 20 pts/see, 220 and 254 nm. MS Detector Waters SQD, ESI (ES+/ES−, 120-1200 amu).

Method L: HPLC-MS method: Waters Alliance UPLC CSH C18, 3.5 μm, 4.6×30 mm at 40° C.; 5% B for 0.2 min, 5% to 100% B in 1.8 minutes; hold 100% B for 1 minute, run time=3.0 min, flow 3 mL/min; Eluents: A=Milli-Q H₂O+10 mM ammonium formate pH=3.8; B=acetonitrile. Waters Alliance HPLC system. UV Detector=Waters 2996 PDA, 198-360 nm. MS Detector=Waters ZQ 2000.

Method M: HPLC-MS method: Waters Alliance UPLC CSH C18, 3.5 μm, 4.6×30 mm at 40° C.; 5% B for 0.5 min, 5% to 100% B in 5.0 minutes; hold 100% B for 0.7 minute, 100% B for 1.5 min, run time=7.0 min, flow 3 mL/min; Eluents: A=Milli-Q H₂O+10 mM ammonium formate, pH 3.8; B=MeCN. Waters Alliance HPLC system. UV Detector=Waters 2996 PDA, 198-360 nm. MS Detector=Waters ZQ 2000.

GCMS method (method Z): The GC-MS system consists of Agilent GCMS 7890B and Detector Channel FID.

### The MS Detector of Acquisition Mode:

Start Time: 2.00 min; End Time: 11.75 min; Acquisition Mode: Scan; Interface Type: EI
Threshold: 150; Scan Speed: 1562; Start m/z: 50.00; End m/z: 600.00; MS Source: 230.00° C.; MS Quad: 150.00° C.; Solvent Cut Time: 2.00 min.

### GC Parameters:

Column: HP-5MS, 30 m×0.25 mm×0.25 μm; Column Oven Temp: 50.0° C.; injection volume: 1 μL;
Column Flow: 1.0 ml/min; Injection temperature: 300° C.; Injection Mode: Split; Split Ratio: 20:1;
Detector temperature: 300° C.; Initial temperature: 50° C. for 0.5 min then 40° C./min to 300° C. for 11.75 min.
Makeup Gas: He; Makeup Flow: 25.0 mL/min; H₂; Flow: 30.0 mL/min; Air Flow: 400.0 mL/min;
Final temperature: 325° C.

### Example 1

### (S)-(2,3-dichloro-6-fluorophenyl)(1-methylcyclopentyl)methanamine

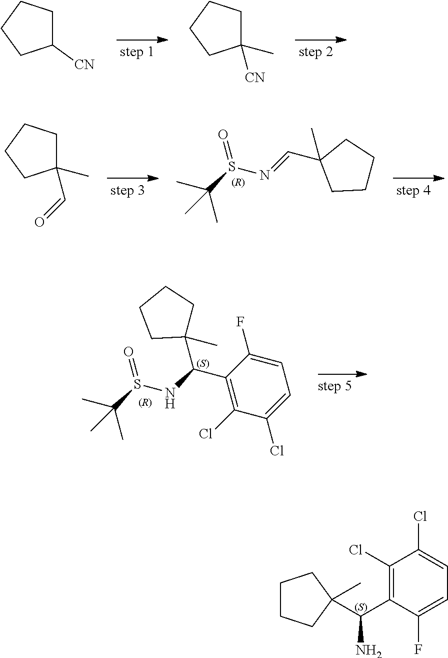

### Step 1. Synthesis of 1-methylcyclopentane-1-carbonitrile

To a solution of LiHMDS (1.00 M, 1.50 L) was a solution of cyclopentanecarbonitrile (130 g, 1.37 mol) in THF (650 mL) added dropwise at −60° C. under N₂. After the addition, the reaction mixture was stirred at −60° C. for 1 hour. Then Mel (111 mL, 1.78 mol) was added dropwise at −60° C. The reaction mixture was allowed to warm to 20° C. and stirred at 20° C. for 12 hours. The mixture was poured into saturated aqueous NH₄Cl solution (2.00 L) and extracted with ethyl acetate (1.50 L*2). The combined organic layer was washed with 1N HCl (1.00 L) and brine (1.50 L*2), dried over Na₂SO₄, filtered and concentrated under reduced pressure to give 1-methylcyclopentane-1-carbonitrile (150 g, crude) as a yellow oil. ¹H NMR: (400 MHz, CDCl₃) δ 2.16-2.14 (m, 2H), 1.85-1.77 (m, 4H), 1.63-1.59 (m, 2H), 1.41 (s, 3H).

### Step 2. Synthesis of 1-methylcyclopentane-1-carbaldehyde

To a solution of DIBAL-H (1.00 M in THF, 1.51 L) was added dropwise a solution of 1-methylcyclopentane-1-carbonitrile (150 g, 1.37 mol) in DCM (150 mL) at −65° C. under N₂. The mixture was stirred at −65° C. for 1 hour and poured into saturated aqueous NH₄Cl solution (5.00 L) under stirring. The pH was adjusted to ˜3 with HCl (6 N, 1.20 L), then extracted with DCM (2.00 L*2). The combined organic layer was washed with brine (1.50 L*2), dried over Na₂SO₄, filtered and concentrated (15° C.) under reduced pressure to give 1-methylcyclopentane-1-carbaldehyde (130 g, crude) as a colorless liquid. The crude product was used in the next step without purification.

### Step 3. Synthesis of (R)-2-methyl-N-((1-methylcyclopentyl)methylene)propane-2-sulfinamide

To a mixture of 1-methylcyclopentane-1-carbaldehyde (120 g, 1.07 mol) in THF (600 mL) was added (R)-2-methylpropane-2-sulfinamide (156 g, 1.28 mol), Ti(OⁱPr)₄ (608 g, 2.14 mol) at 20° C. The mixture was heated to 75° C. and stirred at 75° C. for 2 hours. The mixture was poured into brine (4.00 L). The mixture was filtered, and the filtrate was washed with ethyl acetate (1.50 L*3). The filtrate was washed with brine (2.00 L) and concentrated to give a residue. The residue was purified by column chromatography (SiO₂, petroleum ether/ethyl acetate=10/1 to 5/1) to give (R)-2-methyl-N-((1-methylcyclopentyl)methylene)propane-2-sulfinamide (120 g, 557 mmol) as a yellow oil. ¹H NMR: (400 MHz, CDCl₃) δ 7.95 (s, 1H), 1.94-1.92 (m, 2H), 1.75-1.68 (m, 4H), 1.50-1.46 (m, 2H), 1.22 (s, 3H), 1.19 (s, 9H).

### Step 4. Synthesis of (R)—N—((S)-(2,3-dichloro-6-fluorophenyl)(1-methylcyclopentyl)methyl)-2-methylpropane-2-sulflnamide

To a mixture of 1,2-dichloro-4-fluorobenzene (99.6 g, 604 mmol) in THF (1.00 L) was added dropwise n-BuLi (2.50 M in hexanes, 241 mL) at −65° C. under N₂, then the mixture was stirred at −65° C. for 0.5 hour. To the mixture was added (R)-2-methyl-N-((1-methylcyclopentyl)methylene)propane-2-sulfinamide (100 g, 464 mmol) in THF (100 mL). It was stirred at −65° C. for 1 hour. The mixture was poured into saturated aqueous NH₄Cl solution (10%, 2.00 L) and extracted with ethyl acetate (1.00 L*2). The combined organic layers were washed with brine (1.00 L*2) and concentrated under reduced pressure to give a residue. The residue was purified by preparative HPLC (column: Phenomenex luna C18 250 mm*100 mm, 10 μm; mobile phase A: water (formic acid) B: acetonitrile; B: 60%-80% over 25 min) to give (R)—N—((S)-(2,3-dichloro-6-fluorophenyl)(1-methylcyclopentyl)methyl)-2-methylpropane-2-sulfinamide (120 g, 316 mmol) as a yellow oil. ¹H NMR: (400 MHz, DMSO-d₆) δ 7.66-7.62 (m, 1H), 7.32-7.27 (m, 1H), 5.13 (d, J=8.4 Hz, 1H), 4.91 (d, J=8.4 Hz, 1H), 1.74-1.62 (m, 6H), 1.39-1.36 (m, 1H), 1.26-1.22 (m, 1H), 0.97-0.95 (m, 12H).

### Step 5. Synthesis of (S)-(2,3-dichloro-6-fluorophenyl)(1-methylcyclopentyl)methanamine

To a mixture of (R)—N—((S)-(2,3-dichloro-6-fluorophenyl)(1-methylcyclopentyl)methyl)-2-methylpropane-2-sulfinamide (110 g, 289 mmol) in ethyl acetate (1.10 L) was added HCl in EtOAc (4 M, 275 mL). The mixture was stirred at 20° C. for 1 hour. The mixture was concentrated. To the residue was added H₂O (1.50 L), and it was washed with ethyl acetate (1.00 L*2). To the aqueous layer was added saturated aqueous NaHCO₃ solution (800 mL) until pH=9. The mixture was extracted with ethyl acetate (1.00 L*2) and the combined organic layers were washed with brine (500 mL*2), dried over Na₂SO₄, filtered and concentrated under reduced pressure to give (S)-(2,3-dichloro-6-fluorophenyl)(1-methylcyclopentyl)methanamine (52.23 g, 188 mmol) as a yellow oil. ¹H NMR: (400 MHz, DMSO-d₆) δ 7.59-7.56 (m, 1H), 7.27-7.22 (m, 1H), 4.32 (s, 1H), 2.10 (s, 2H), 1.79-1.59 (m, 6H), 1.29-1.26 (m, 1H), 1.14-1.11 (m, 1H), 0.86 (d, J=2.4 Hz, 3H).

### Example 2

### (2r,4r)-6,8-dioxo-5,7-diazaspiro[3.4]octane-2-carboxylic acid

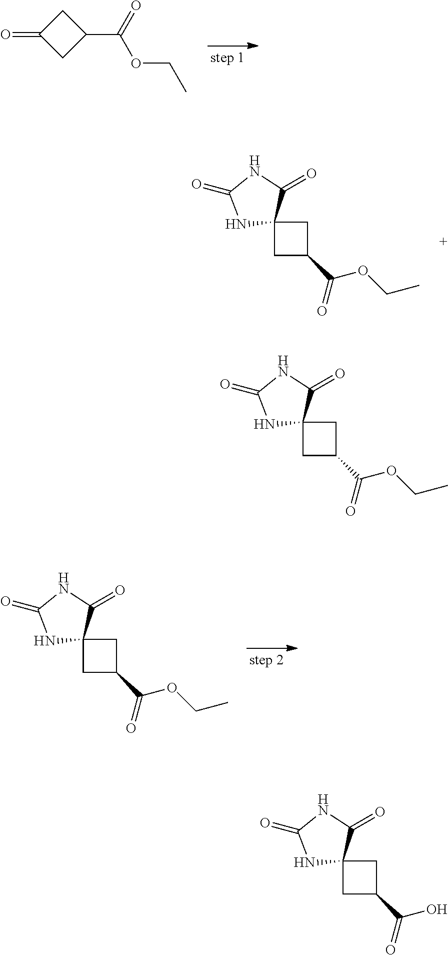

### Step 1. Synthesis of Ethyl 6,8-dioxo-5,7-diazaspiro[3.4]octane-2-carboxylate

To a mixture of ethyl 3-oxocyclobutane-1-carboxylate (113 g, 791 mmol), (NH₄)₂CO₃ (114 g, 1.19 mol) in EtOH (1.50 L) and H₂O (500 mL) was added NaCN (38.8 g, 791 mmol) at 20° C. The mixture was heated to 35° C. and stirred at 35° C. for 12 hours. Four such batches were combined. The reaction mixture was extracted with ethyl acetate (2.00 L*3). The combined organic layers were dried over anhydrous Na₂SO₄, filtered and concentrated to give a white solid. It was purified by preparative HPLC (column: Phenomenex luna C18 250*50 mm*10 μm; mobile phase A: water (0.1% TFA) B: acetonitrile; gradient: B % 10%-35% over 21 minutes). The eluent was concentrated to remove most of acetonitrile and extracted with ethyl acetate (5.00 L*6). The combined organic layer was dried over anhydrous sodium sulfate, filtered and concentrated under vacuum. Ethyl (2r,4r)-6,8-dioxo-5,7-diazaspiro[3.4]octane-2-carboxylate (90.0 g, 411 mmol) was obtained as a white solid. ¹H NMR: (400 MHz, DMSO-d6) δ 10.6 (s, 1H), 8.48 (s, 1H), 8.46 (s, 1H), 4.12-4.05 (m, 2H), 3.25-3.18 (m, 1H), 2.69-2.65 (m, 2H), 2.41-2.34 (m, 2H), 1.19 (t, J=6.8 Hz, 3H).

Ethyl (2s,4s)-6,8-dioxo-5,7-diazaspiro[3.4]octane-2-carboxylate (150 g, 66% purity, containing 30% ethyl (2r,4r)-6,8-dioxo-5,7-diazaspiro[3.4]octane-2-carboxylate) was also obtained.

### Step 2. Synthesis of (2r,4r)-6,8-dioxo-5,7-diazaspiro[3.4]octane-2-carboxylic acid

To a solution of ethyl (2r,4r)-6,8-dioxo-5,7-diazaspiro[3.4]octane-2-carboxylate (90.0 g, 424 mmol) in MeOH (450 mL) and H₂O (200 mL) was added LiOH·H₂O (44.5 g, 1.06 mol) at 20° C. The mixture was stirred at 20° C. for 1 hour. The mixture was adjusted to pH=1˜2 with 3 N HCl. The precipitate was collected by filtration. The filter cake was triturated with ethyl acetate (300 mL) at 25° C. for 2 hours and filtered. The filter cake was dried over vacuum to afford (2r,4r)-6,8-dioxo-5,7-diazaspiro[3.4]octane-2-carboxylic acid (52.0 g, 279 mmol) as a white solid. ¹H NMR: (400 MHz, DMSO-d₆) δ 12.33 (s, 1H), 10.60 (s, 1H), 8.48 (s, 1H), 3.16-3.13 (m, 1H), 2.70-2.64 (m, 2H), 2.35-2.29 (m, 2H).

### Example 3

### (2r,4S)—N—((S)-(2,3-dichloro-6-fluorophenyl)(1-methylcyclopentyl)methyl)-6,8-dioxo-5,7-diazaspiro[3.4]octane-2-carboxamide

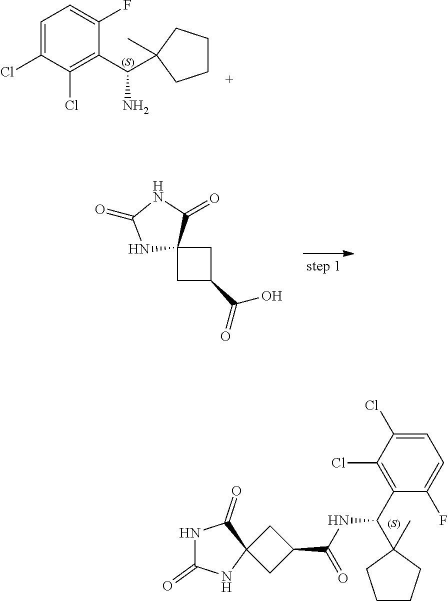

### Step 1. Synthesis of (2r,4S)—N—((S)-(2,3-dichloro-6-fluorophenyl)(1-methylcyclopentyl)methyl)-6,8-dioxo-5,7-diazaspiro[3.4]octane-2-carboxamide

A mixture of (S)-(2,3-dichloro-6-fluorophenyl)(1-methylcyclopentyl)methanamine (40 mg, 0.14 mmol), (2r,4r)-6,8-dioxo-5,7-diazaspiro[3.4]octane-2-carboxylic acid (27 mg, 0.14 mmol), TEA (44 mg, 0.43 mmol) and T₃P (0.14 g, 50% wt, 0.22 mmol) in DMF (1 mL) was stirred at 25° C. for 1 h. The reaction was quenched with water (5 ml) and extracted with ethyl acetate (10 ml*3). The combined organic layers were washed with brine (5 ml). The mixture was dried over Na₂SO₄ and concentrated. The residue was purified by C18 flash chr omatography (CH₃CN/water, 25%-60% CH₃CN over 20 min) to afford (2r,4S)—N—((S)-(2,3-dichloro-6-fluorophenyl)(1-methylcyclopentyl)methyl)-6,8-dioxo-5,7-diazaspiro[3.4]octane-2-carboxamide (43.1 mg, 97.4 μmol) as a white solid. ¹H NMR (400 MHz, DMSO-d6) δ 10.57 (s, 1H), 8.62 (s, 1H), 8.23 (d, J=8.6 Hz, 1H), 7.61 (dd, J=8.9, 5.0 Hz, 1H), 7.25 (dd, J=10.8, 9.0 Hz, 1H), 5.50 (d, J=8.5 Hz, 1H), 3.30-3.26 (m, 1H), 2.71-2.53 (m, 2H), 2.21 (dd, J=24.5, 11.2 Hz, 2H), 1.59 (s, 6H), 1.37 (s, 1H), 1.27 (s, 1H), 0.96 (d, J=2.8 Hz, 3H). LC MS RT 0.928 min, [M+H]⁺ 442, LCMS method C.

### Example 4

### (S)-(3-chlorophenyl)(cyclopentyl)methanamine

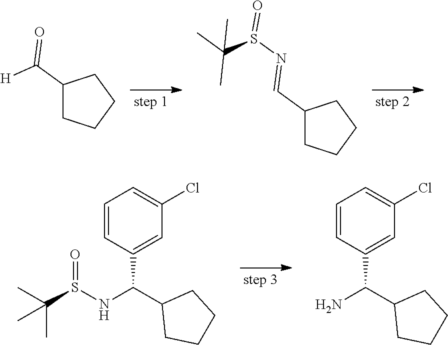

### Step 1. Synthesis of (R)—N-(cyclopentylmethylene)-2-methylpropane-2-sulfinamide

To a solution of cyclopentanecarbaldehyde (112 g, 1.15 mol) and (R)-2-methylpropane-2-sulfinamide (167 g, 1.38 mol) in THF (560 mL) was added Ti(OⁱPr)₄ (651 g, 2.29 mol) under N₂ atmosphere at 25° C. The mixture was heated to 75° C. and stirred at 75° C. for 2 hours. Two batches were carried out in parallel and combined in the workup. After cooling to room temperature, to the mixture was added brine (3.00 L). The suspension was filtered. The filter cake was washed with ethyl acetate (5.00 L*2). The organic phase was separated and the aqueous phase was extracted with ethyl acetate (3.00 L). The combined organic phase was washed with brine (3.00 L), dried over Na₂SO₄, filtered and concentrated. The residue was purified by column chromatography (SiO₂, petroleum ether:ethyl acetate 1:0 to 10:1). (R)—N-(cyclopentylmethylene)-2-methylpropane-2-sulfinamide (357 g, 1.77 mol) was obtained as a yellow oil. ¹H NMR: (400 MHz, CDCl₃) δ 8.00 (d, J=5.6 Hz, 1H), 2.98-2.94 (m, 1H), 1.94-1.83 (m, 2H), 1.77-1.62 (m, 6H), 1.19 (s, 9H)

### Step 2. Synthesis of (R)—N—((S)-(3-chlorophenyl)(cyclopentyl)methyl)-2-methylpropane-2-sulfinamide

Two batches were carried out in parallel and combined in the workup. To a solution of (R)—N-(cyclopentylmethylene)-2-methylpropane-2-sulfinamide (160 g, 795 mmol) and 1-bromo-3-chlorobenzene (228 g, 1.19 mol) in THF (800 mL) was added n-BuLi (2.50 M in hexanes, 477 mL) dropwise at −70˜−60° C. under N₂. The reaction was stirred at −70˜−60° C. for 2 hours.

The mixture was poured into saturated NH₄Cl solution (5.00 L) and extracted with ethyl acetate (2.00 L*3). The combined organic phase was washed with brine (2.00 L), dried over Na₂SO₄, filtered and concentrated to give a yellow oil (563 g). The crude product was used in the next step without purification. LCMS: RT 1.030 min, [M+H]⁺ 314.1, LCMS method I.

### Step 3. Synthesis of (S)-(3-chlorophenyl)(cyclopentyl)methanamine

Two equal batches were carried out in parallel. To a solution of (R)—N—((S)-(3-chlorophenyl)(cyclopentyl)methyl)-2-methylpropane-2-sulfinamide (264 g, 757 mmol) in ethyl acetate (2.60 L) was added HCl in EtOAc (4.00 M, 473 mL) at 25° C. The mixture was stirred at 25° C. for 1 hour. A large amount of white solid was formed. The two batches of reaction mixture were combined. The suspension was concentrated to 4.0 L and the suspension was filtered. The filter cake was washed with ethyl acetate (200 mL*2). The filter cake was partitioned between ethyl acetate (2.00 L) and saturated NaHCO₃ solution (2.50 L). The suspension was stirred for 10 minutes until the solid disappeared. The organic phase was separated and the aqueous phase was extracted with ethyl acetate (1.00 L*2). The combined organic phase was washed with brine (2.00 L), dried over Na₂SO₄, filtered and concentrated to give (S)-(3-chlorophenyl)(cyclopentyl)methanamine (220 g, crude) as a yellow oil. To a solution of (2R,3R)-2,3-dihydroxysuccinic acid (80.0 g, 535 mmol) in MeOH (1.30 L) was added crude (S)-(3-chlorophenyl)(cyclopentyl)methanamine (110 g, 525 mmol) at 25° C. The reaction was stirred at 25° C. for 10 minutes, and a white solid was formed. The reaction mixture was set aside for 3 hours. The suspension was filtered. The filter cake was washed with methanol (100 mL*2), dried under vacuum to give a solid (260 g, ee %=98.0%). The product was diluted with methanol (1.00 L) and heated at 80° C. for 1.0 hour until the solid fully dissolved. The reaction was set aside for 72 hours. A white solid precipitated. The reaction mixture was filtered and the filter cake was washed with methanol (100 mL*2). The filter cake was partitioned between saturated aq. NaHCO₃ (2.50 L) and ethyl acetate (2.00 L). The organic phase was separated. The aqueous phase was extracted with ethyl acetate:methanol=10:1 (2.00 L*2). The combined organic layers were dried over Na₂SO₄, filtered and concentrated under reduced pressure to give (S)-(3-chlorophenyl)(cyclopentyl)methanamine (94.0 g, ee %=100%). ¹H NMR: (400 MHz DMSO-d₆) δ 7.40 (s, 1H), 7.32-7.22 (m, 3H), 3.54 (d J=8.4 Hz, 1H), 2.18 (s, 2H), 2.00-1.90 (m, 1H), 1.79-1.71 (m, 1H), 1.60-1.45 (m, 3H), 1.42-1.30 (m, 2H), 1.25-1.17 (m, 1H), 1.11-1.02 (m, 1H).

### Example 5

### (S)-(3-chloro-2,6-difluorophenyl)(4-fluorobicyclo[2.2.1]heptan-1-yl)methanamine hydrochloride

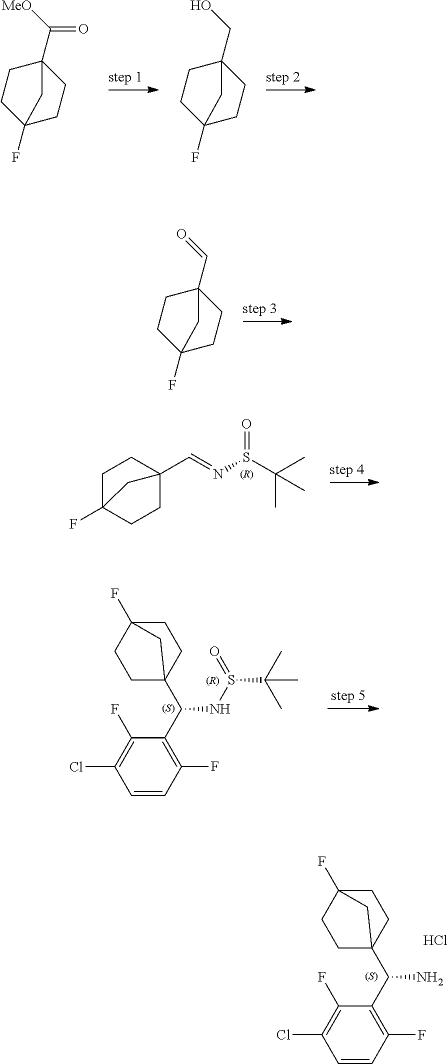

### Step 1. Synthesis of (4-fluorobicyclo[2.2.1]heptan-1-yl)methanol

To a solution of methyl 4-fluorobicyclo[2.2.1]heptane-1-carboxylate (165 g, 958 mmol) in THF (1.65 L) was added dropwise LiAlH₄ (2.5 M in THF, 460 mL) at 0˜10° C. The mixture was warmed to 20° C. and stirred at 20° C. for 2 hours. The reaction mixture was slowly poured into 1 M aqueous HCl (5.00 L) and extracted with ethyl acetate (5.00 L*2). The organic phase was washed with brine (5.00 L), dried over anhydrous Na₂SO₄, filtered and concentrated under vacuum to give (4-fluorobicyclo[2.2.1]heptan-1-yl)methanol (146 g, crude) as a light yellow oil. ¹H NMR (400 MHz, CDCl₃) δ 3.61 (s, 2H), 2.05-1.93 (m, 2H), 1.85-1.72 (m, 4H), 1.52-1.39 (m, 4H).

### Step 2. Synthesis of 4-fluorobicyclo[2.2.1]heptane-1-carbaldehyde

To a solution of (4-fluorobicyclo[2.2.1]heptan-1-yl)methanol (150 g, 1.04 mol) in DCM (1.13 L) was added DMSO (244 mL, 3.12 mol), TEA (724 mL, 5.20 mol) at 20° C. The mixture was cooled to 0˜5° C. SO₃·Py (745 g, 4.68 mol) was added to the mixture at 0˜5° C. The mixture was warmed to 20° C. and stirred at 20° C. for 2 hours. The mixture was poured into water (5.00 L) and extracted with DCM (5.00 L*2). The organic phase was dried over anhydrous Na₂SO₄, filtered and concentrated under vacuum at 25° C. The residue was dissolved in ethyl acetate (2.00 L). The organic phase was washed with 1 N aqueous HCl (1.50 L*2), brine (1.50 L), dried over anhydrous Na₂SO₄, filtered and concentrated under vacuum to give 4-fluorobicyclo[2.2.1]heptane-1-carbaldehyde (112 g, crude) as a yellow oil. ¹H NMR (400 MHz, CDCl₃) δ 9.72 (s, 1H), 2.21-2.10 (m, 2H), 2.04-1.95 (m, 2H), 1.90-1.82 (m, 4H), 1.65-1.58 (m, 2H).

### Step 3. Synthesis of (R)—N-((4-fluorobicyclo[2.2.1]heptan-1-yl)methylene)-2-methylpropane-2-sulfinamide

To a solution of 4-fluorobicyclo[2.2.1]heptane-1-carbaldehyde (110 g, 774 mmol), (R)-2-methylpropane-2-sulfinamide (93.8 g, 774 mmol) in THF (1.10 L) was added, followed by Ti(OⁱPr)₄ (440 g, 1.55 mol) at 25° C. The mixture was heated to 75° C. and stirred at 75° C. for 2 hours. The mixture was cooled to 25° C., diluted with ethyl acetate (4.00 L) and poured into water (4.00 L). The mixture was filtered. The filtrate was separated, and the organic phase was washed with brine (3.00 L), dried over anhydrous Na₂SO₄, filtered and concentrated under vacuum. The residue was combined with another batch (which started with 20.0 g of the aldehyde) and purified by column chromatography (SiO₂, petroleum ether:ethyl acetate 1:0 to 10:1) to give (R)—N—((S)-(3-chloro-2,6-difluorophenyl)(4-fluorobicyclo[2.2.1]heptan-1-yl)methyl)-2-methylpropane-2-sulfinamide (150 g, 599 mmol) as a white solid. ¹H NMR (400 MHz, CDCl₃) δ 8.09 (s, 1H), 2.13-1.94 (m, 4H), 1.91-1.80 (m, 4H), 1.72-1.63 (m, 2H), 1.19 (s, 9H); ¹⁹F NMR (376 MHz, CDCl₃) δ −176.81

### Step 4. Synthesis of (R)—N—((S)-(3-chloro-2,6-difluorophenyl)(4-fluorobicyclo[2.2.1]heptan-1-yl)methyl)-2-methylpropane-2-sulfinamide

To a solution of 1-chloro-2,4-difluorobenzene (8.97 g, 60.4 mmol) in THF (114 mL) was added n-BuLi (2.50 M in hexanes, 24.1 mL) at −65° C. The mixture was stirred at −65° C. for 0.5 hour, then a solution of (R)—N-((4-fluorobicyclo[2.2.1]heptan-1-yl)methylene)-2-methylpropane-2-sulfinamide (11.4 g, 46.4 mmol) in THF (114 mL) was added at −65° C. The mixture was stirred at −65° C. for 2 hours. The reaction mixture was poured into saturated NH₄Cl solution (500 mL). The aqueous phase was extracted with ethyl acetate (300 mL*2). The combined organic phase was washed with saturated brine (500 mL*2), dried over anhydrous Na₂SO₄, filtered and concentrated in vacuum to give (R)—N—((S)-(3-chloro-2,6-difluorophenyl)(4-fluorobicyclo[2.2.1]heptan-1-yl)methyl)-2-methylpropane-2-sulfinamide (20.0 g, crude) as a yellow solid. LCMS RT 0.598 min, [M+H]⁺ 394.1, LCMS method J.

### Step 5. Synthesis of (S)-(3-chloro-2,6-difluorophenyl)(4-fluorobicyclo[2.2.1]heptan-1-yl)methanamine hydrochloride

To a solution of (R)—N—((S)-(3-chloro-2,6-difluorophenyl)(4-fluorobicyclo[2.2.1]heptan-1-yl)methyl)-2-methylpropane-2-sulfinamide (20.0 g, 50.7 mmol) in MeOH (100 mL) was added HCl (4.00 N in MeOH, 100 mL) at 25° C. The mixture was stirred at 25° C. for 1 hour. The reaction mixture was concentrated in vacuum to give the crude product. The residue was triturated with ethyl acetate (100 mL) at 20° C. for 30 minutes, filtered and the filter cake was dried under vacuum at 50° C. to give (S)-(3-chloro-2,6-difluorophenyl)(4-fluorobicyclo[2.2.1]heptan-1-yl)methanamine hydrochloride (10.0 g, 30.6 mmol) as a white solid. LCMS: RT 0.423 min, [M+H]⁺ 290.1, LCMS method J; ¹H NMR (400 MHz, MeOH-d4) δ 7.69-7.64 (m, 1H), 7.22-7.17 (m, 1H), 3.32-3.31 (m, 1H), 1.99-1.81 (m, 8H), 1.77-1.57 (m, 2H).

### Example 6

### (1S,3S,4R)-3-((tert-butoxycarbonyl)amino)-4-hydroxycyclopentane-1-carboxylic acid

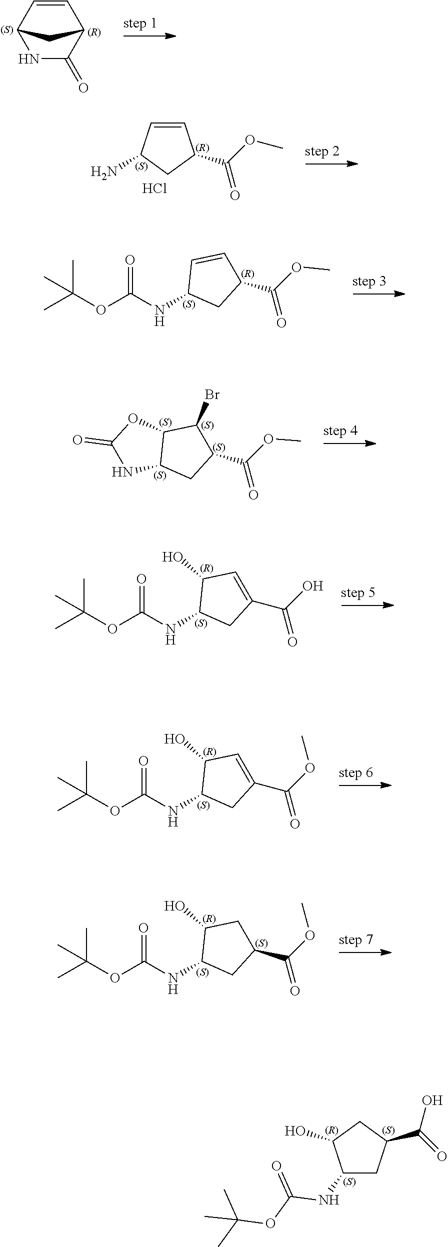

### Step 1. Synthesis of methyl (1R,4S)-4-aminocyclopent-2-ene-1-carboxylate hydrochloride

To a solution of (1S,4R)-2-azabicyclo[2.2.1]hept-5-en-3-one (535 g, 4.90 mol) in MeOH (1605 mL) was added SOCl₂ (213 mL, 2.94 mol) at 0° C. and the solution was stirred at 0° C. for 2 hours. The solution was concentrated under vacuum. The crude product was triturated with MTBE (1000 mL) at 20° C. for 30 minutes to give methyl (1R,4S)-4-aminocyclopent-2-ene-1-carboxylate hydrochloride (860 g, 4.84 mol). ¹HNMR: (400 MHz DMSO-d6) δ 8.37 (s, 3H), 6.05-6.07 (m, 1H), 5.87-5.89 (m, 1H), 4.16 (s, 1H), 3.68-3.70 (m, 1H), 3.64 (m, 3H), 2.53-2.59 (m, 1H), 1.90-1.97 (m, 1H).

### Step 2. Synthesis of methyl (1R,4S)-4-((tert-butoxycarbonyl)amino)cyclopent-2-ene-1-carboxylate

To a solution of methyl (1R,4S)-4-aminocyclopent-2-ene-1-carboxylate hydrochloride (750 g, 4.22 mol) and Boc₂O (919 g, 4.22 mol) in DCM (4.5 L) was added TEA (728 mL, 4.22 mol) at 0° C. and the solution was stirred at 25° C. for 12 hours. The reaction reaction was quenched by water (1000 mL) and extracted with dichloromethane (500 mL*2). The combined organic layers were washed with brine (500 mL), dried over sodium sulfate, filtered and concentrated under reduced pressure to give methyl (1R,4S)-4-((tert-butoxycarbonyl)amino)cyclopent-2-ene-1-carboxylate (1.00 kg, 4.13 mol). ¹HNMR: (400 MHz CDCl₃) b 5.83-5.87 (m, 1H), 4.88 (s, 1H), 4.77 (s, 1H), 3.69 (s, 3H), 3.45-3.47 (m, 1H), 2.45-2.53 (m, 1H), 1.83-1.87 (m, 1H), 1.25 (s, 9H).

### Step 3. Synthesis of methyl (3aS,5S,6S,6aS)-6-bromo-2-oxohexahydro-2H-cyclopenta[d]oxazole-5-carboxylate

To a solution of methyl (1R,4S)-4-((tert-butoxycarbonyl)amino)cyclopent-2-ene-1-carboxylate (630 g, 2.61 mol) in THF (2.0 L) and H₂O (189 mL) was added NBS (511 g, 2.87 mol) at 0° C. and the solution was stirred at 25° C. for 12 hours. The reaction was concentrated under reduced pressure. The residue was dissolved in dichloromethane (2000 mL) and washed sequentially with HCl (500 mL, 1 M), saturated Na₂SO₃ (aq., 1000 mL) and brine (500 mL) before drying over MgSO₄. The organic phase was concentrated under reduced pressure to give methyl (3aS,5S,6S,6aS)-6-bromo-2-oxohexahydro-2H-cyclopenta[d]oxazole-5-carboxylate (854 g, 3.23 mol) as a white solid. ¹HNMR: (400 MHz CDCl₃) δ 6.27 (s, 1H), 5.13-5.15 (d, J=8 Hz, 1H), 4.76 (s, 1H), 4.40-4.43 (m, 1H), 3.74 (s, 1H), 3.19-3.23 (m, 1H), 2.23-2.54 (m, 2H).

### Step 4. Synthesis of (3R,4S)-4-((tert-butoxycarbonyl)amino)-3-hydroxycyclopent-1-ene-1-carboxylic acid

Two reactions were run in parallel. To a solution of methyl (3aS,5S,6S,6aS)-6-bromo-2-oxohexahydro-2H-cyclopenta[d]oxazole-5-carboxylate (375 g, 1.42 mol) in H₂O (1.6 L) and MeOH (1.6 L) was added KOH (318 g, 5.68 mol) at 0° C. and the solution was stirred at 90° C. for 12 hours. The above solution was concentrated and dissolved in THF (700 mL) and Boc₂O (309 g, 1.42 mol) was added. The solution was stirred at 20° C. for 4 h. The two reactions were combined for work up. The resulting mixture was concentrated in vacuum and then ethyl acetate (500 mL) and H₂O (400 mL) were added. The aqueous phase was separaaed and the pH was adjusted to 3 with HCl (1 M). The solution was extracted with ethyl acetate (1000 mL*2). The combined organic layers were washed with brine (500 mL), dried over sodium sulfate, filtered and concentrated under reduced pressure to give (3R,4S)-4-((tert-butoxycarbonyl)amino)-3-hydroxycyclopent-1-ene-1-carboxylic acid (600 g, 2.47 mol) as a white solid. ¹HNMR (400 MHz DMSO-d6) δ 12.49 (s, 1H), 6.51 (s, 1H), 6.31-6.33 (m, 1H), 5.03 (s, 1H), 4.50 (s, 1H), 3.97-4.00 (m, 1H), 2.55-2.61 (m, 1H), 2.31-2.37 (m, 1H), 1.45 9s,9H).

### Step 5. Synthesis of methyl (3R,4S)-4-((tert-butoxycarbonyl)amino)-3-hydroxycyclopent-1-ene-1-carboxylate

To a solution of (3R,4S)-4-((tert-butoxycarbonyl)amino)-3-hydroxycyclopent-1-ene-1-carboxylic acid (400 g, 1.64 mol) in MeOH (2.8 L) was added TEA (389 mL, 2.80 mol). The mixture was cooled to 0° C. and methyl chloroformate (216 mL, 2.80 mol) was added dropwise. The mixture was stirred at 0° C. for 1 hour, then stirred at 15° C. for 12 hours. The reaction mixture was concentrated under reduced pressure. The residue was diluted with DCM (1000 mL). The organic layer was washed with 1 M potassium hydrogen sulfate (aq) (500 ml*2), saturated NaHCO₃ solution (500 ml*2), dried over Na₂SO₄, filtered and concentrated under reduced pressure to give methyl (3R,4S)-4-((tert-butoxycarbonyl)amino)-3-hydroxycyclopent-1-ene-1-carboxylate (300 g, 1.10 mol) as a white solid. ¹HNMR: (400 MHz CDCl₃) δ 6.69 (s, 1H), 5.134 (d, J=8 Hz, 1H), 4.76 (s, 1H), 4.24 (s, 1H), 3.75 (s, 3H), 2.88-2.94 (m, 1H), 2.03-2.51 (m, 2H), 1.26 (s, 9H).

### Step 6. Synthesis of methyl (1S,3S,4R)-3-((tert-butoxycarbonyl)amino)-4-hydroxycyclopentane-1-carboxylate

Three reactions were run in parallel. [(S,S)-(Me-DuPHOS)—Rh(COD)]BF₄ (2.36 g, 3.89 mmol) was added to a degassed solution of methyl (3R,4S)-4-((tert-butoxycarbonyl)amino)-3-hydroxycyclopent-1-ene-1-carboxylate (50 g, 194 mmol) in MeOH (250 mL). The reaction mixture was transferred to a hydrogenation bomb and after purging with N₂ and then H₂, an H₂ pressure of 2 MPa was applied and the reaction was stirred for 14 hours at 25° C. The pressure was released and the bomb was purged with N₂. Concentration of the reaction mixture gave a residue which was dissolved in DCM (500 mL). Addition of silica (150 g) with stirring removed the catalyst from the reaction and filtration and concentration of the organic solution gave methyl (1S,3S,4R)-3-((tert-butoxycarbonyl)amino)-4-hydroxycyclopentane-1-carboxylate (150 g, 501 mmol). HNMR: (400 MHz CDCl₃) δ 4.81 (s, 1H), 4.28 (s, 1H), 3.67 (s, 3H), 3.08-3.15 (m, 1H), 2.07-2.27 (m, 1H), 2.02-2.05 (m, 2H), 1.84-1.86 (m, 2H), 1.44 (s, 9H).

### Step 7. Synthesis of Synthesis of (1S,3S,4R)-3-((tert-butoxycarbonyl)amino)-4-hydroxycyclopentane-1-carboxylic acid

To a solution of methyl (1S,3S,4R)-3-((tert-butoxycarbonyl)amino)-4-hydroxycyclopentane-1-carboxylate (170 g, 655 mmol) in MeOH (200 mL) and H₂O (200 mL) was added LiOH·H₂O (33.0 g, 786 mmol) at 20° C. and the suspension was stirred at 20° C. for 12 hours. After concentration in vacuo, the pH of the solution was adjusted to 4 with citric acid and it was extracted with ethyl acetate (100.0 mL*3). The combined organic layers were washed with brine (10.0 mL), dried over sodium sulfate, filtered and concentrated under reduced pressure. The crude product was triturated with petroleum ether:MTBE 3:1 (200 mL) at 20° C. for 30 minutes to give (1S,3S,4R)-3-((tert-butoxycarbonyl)amino)-4-hydroxycyclopentane-1-carboxylic acid (93.5 g, 379 mmol). The mother liquor was purified by column chromatography (SiO₂, petroleum ether/ethyl acetate 5/1 to 0/1) to give more (1S,3S,4R)-3-((tert-butoxycarbonyl)amino)-4-hydroxycyclopentane-1-carboxylic acid (18.0 g, 72.9 mmol). ¹HNMR: (400 MHz CDCl₃) δ 5.11 (s, 1H), 4.25 (s, 1H), 4.00 (s, 1H), 3.11 (s, 1H), 2.24-2.32 (m, 1H), 1.91-2.09 (m, 2H), 1.82-1.88 (s, 1H), 1.43 (s, 9H).

### Example 7

### (3S,4R)-3-((tert-butoxycarbonyl)amino)-4-hydroxycyclopentane-1-carboxylic acid

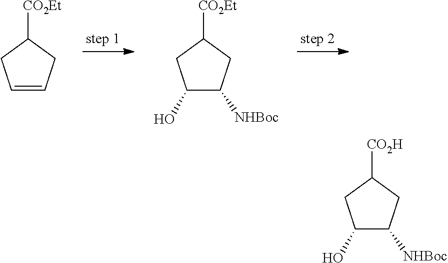 [SMILES: CCOC(=O)C1CC=CC1 ; CCOC(=O)C1C[C@@H](O)[C@@H](NC(=O)OC(C)(C)C)C1 ; CC(C)(C)OC(=O)N[C@H]1CC(C(=O)O)C[C@H]1O]

### Step 1. Synthesis of ethyl (3S,4R)-3-((tert-butoxycarbonyl)amino)-4-hydroxycyclopentane-1-carboxylate

To a mixture of BocNH₂ (98.4 g, 840 mmol) in n-propanol (690 mL) was added NaOH (0.5 M in water, 949 mL), tert-butyl hypochlorite (88.23 g, 813 mmol) at 25° C. The mixture was stirred at 25° C. for 30 minutes. A solution of ethyl cyclopent-3-ene-1-carboxylate (38.0 g, 271 mmol) and (DHQD)₂AQN (4.45 g, 5.42 mmol) in n-propanol (450 mL) was added, followed by K₂[OsO₂(OH)₄](2.00 g, 5.42 mmol) in aqueous NaOH solution (0.5 M, 152 mL) at 25° C. The mixture was stirred at 25° C. for 12 hours. The mixture was poured into H₂O (2.00 L) with stirring. The aqueous phase was extracted with ethyl acetate (1.00 L*3). The combined organic phase was dried over Na₂SO₄, filtered and concentrated under reduced pressure. The residue was purified by column chromatography (SiO₂, petroleum ether:ethyl acetate 30:1 to 0:1) and the fraction was concentrated under reduced pressure. The crude product was purified by preparative HPLC. The combined fractions were concentrated and the pH was adjusted to 7 with saturated aqueous NaHCO₃ solution. The solution was extracted with ethyl acetate (1.00 L*3). The combined organic layer was washed with brine (500 mL*2), dried over Na₂SO₄, filtered and concentrated under reduced pressure to give ethyl (3S,4R)-3-((tert-butoxycarbonyl)amino)-4-hydroxycyclopentane-1-carboxylate (42.0 g, 152 mmol) as a yellow oil. ¹H NMR (400 MHz, CDCl₃) δ 4.89 (d, J=6.4 Hz, 1H), 4.28 (s, 1H), 4.20-4.08 (m, 2H), 4.05-3.90 (m, 1H), 3.14-3.01 (m, 1H), 2.27-2.20 (m, 1H), 2.15-1.98 (m, 2H), 1.89-1.81 (m, 1H), 1.45 (s, 9H), 1.32-1.21 (m, 3H).

### Step 2. Synthesis of (3S,4R)-3-((tert-butoxycarbonyl)amino)-4-hydroxycyclopentane-1-carboxylic acid

This step was done similarly to step 7 in Example 6.

### Example 8

### (2s,4r)-6-oxo-5-azaspiro[3.4]octane-2-carboxylic acid and (2r,4s)-6-oxo-5-azaspiro[3.4]octane-2-carboxylic acid

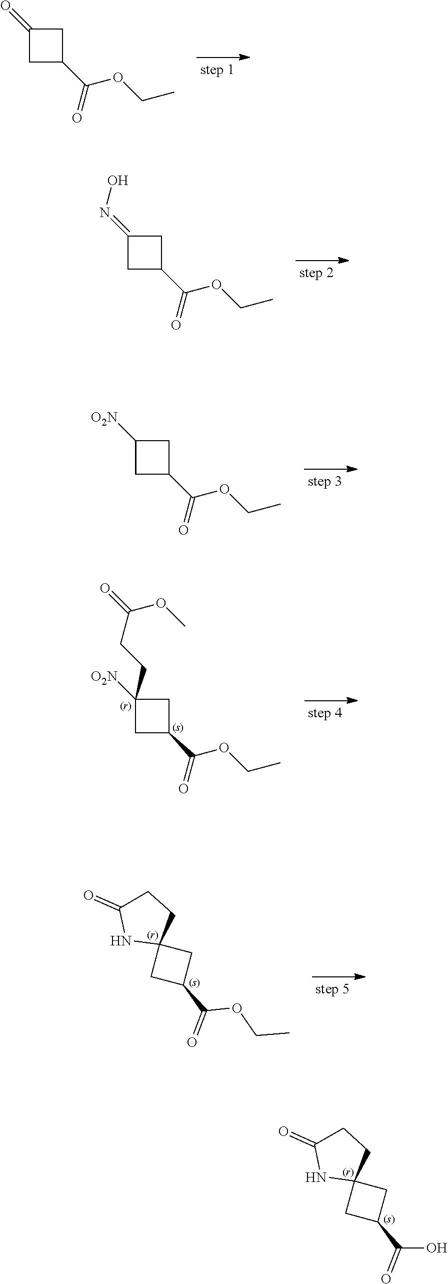

### Step 1. Synthesis of ethyl 3-(hydroxyimino)cyclobutane-1-carboxylate

To a solution of ethyl 3-oxocyclobutane-1-carboxylate (150 g, 1.06 mol) in EtOH (1.50 L) was added NH₂OH·HCl (90.0 g, 1.30 mol) and NaOAc (106 g, 1.29 mol) at 20° C. The reaction mixture was heated to 90° C. and stirred at 90° C. for 15 hours. The reaction mixture was filtered and the filtrate was concentrated under reduced pressure. The residue was diluted with H₂O (1.00 L) and extracted with ethyl acetate (1.00 L*3). The combined organic layers were washed with brine (500 mL*3), dried over Na₂SO₄ and concentrated under reduced pressure. The residue was purified by column chromatography (silica gel, petroleum ether:ethyl acetate 30:1 to 1:1) to give ethyl 3-(hydroxyimino)cyclobutane-1-carboxylate (155 g, 986 mmol) as a colorless oil. ¹H NMR: (400 MHz, CDCl₃) δ 8.36 (br s, 1H), 4.18 (q, J=7.2 Hz, 2H), 3.21-3.16 (m, 5H), 1.28 (t, J=7.2 Hz, 3H).

### Step 2. Synthesis of ethyl 3-nitrocyclobutane-1-carboxylate

Two batches were carried out in parallel. To a mixture of ethyl 3-(hydroxyimino)cyclobutane-1-carboxylate (100 g, 636 mmol) in acetonitrile (600 mL) were Na₂HPO₄ (452 g, 3.18 mol) and urea-hydrogen peroxide (91.5 g, 973 mmol) added at 20° C., then to the mixture was added dropwise a solution of TFAA (266 mL, 1.91 mol) in acetonitrile (400 mL) at 30˜40° C. The mixture was heated to 80° C. and stirred at 80° C. for 1 hour. The two batches were worked up together. The reaction mixture was poured into H₂O (3.00 L) and extracted with ethyl acetate (1.50 L*2). The combined organic layers were washed by Na₂SO₃ solution (10%, 1.50 L*2), brine (1.00 L*2), concentrated under reduced pressure to give ethyl 3-nitrocyclobutane-1-carboxylate (130 g, 751 mmol) as a yellow oil. ¹H NMR: (400 MHz, CDCl₃) δ 5.11-4.82 (m, 1H), 4.22-4.16 (m, 2H), 3.44-3.25 (m, 1H), 2.97-2.80 (m, 4H), 1.32-1.25 (m, 3H).

### Step 3. Synthesis of ethyl (1s,3r)-3-(3-methoxy-3-oxopropyl)-3-nitrocyclobutane-1-carboxylate

To a mixture of ethyl 3-nitrocyclobutane-1-carboxylate (120 g, 693 mmol) in acetonitrile (1.20 L) was added methyl acrylate (246 mL, 2.73 mol) and DBU (104 mL, 693 mmol) dropwise at 0˜10° C. The mixture was warmed to 20° C. and stirred for 2 hours. The reaction mixture was quenched with aqueous NH₄Cl solution (10%, 3.00 L) and extracted with ethyl acetate (2.00 L*2). The combined organic layers were washed by brine (1.50 L*2) and concentrated under reduced pressure The residue was purified by preparative HPLC (column: Welch Ultimate XB-CN 250*50 mm, 10 μm; mobile phase A: hexane, mobile phase B: EtOH; gradient: 7% B isocratic) to give ethyl (1s,3r)-3-(3-methoxy-3-oxopropyl)-3-nitrocyclobutane-1-carboxylate

(38.0 g) as a yellow oil. ¹H NMR: (400 MHz, CDCl₃) δ 4.21-4.12 (m, 2H), 3.69 (s, 3H), 3.26-3.18 (m, 1H), 3.09-3.03 (m, 2H), 2.64-2.59 (m, 2H), 2.48-2.44 (m, 2H), 2.31-2.27 (m, 2H), 1.28 (t, J=7.2 Hz, 3H).

### Step 4. Synthesis of ethyl (2s,4r)-6-oxo-5-azaspiro[3.4]octane-2-carboxylate

To a mixture of compound ethyl (1s,3r)-3-(3-methoxy-3-oxopropyl)-3-nitrocyclobutane-1-carboxylate (38.0 g, 147 mmol) in EtOH (570 mL) was added acetic acid (83.8 mL, 1.47 mol) at 20° C., then iron powder (40.9 g, 733 mmol) was added to the mixture in portions at 50° C. The mixture was stirred at 50° C. for 12 hours. The mixture was cooled to 25° C. To the mixture was added H₂O (500 mL) and it was filtered. The filtrate was concentrated to remove EtOH. Then the mixture was extracted with ethyl acetate (500 mL*2). The combined organic layers were washed with brine (500 mL*2), aqueous NaHCO₃ solution (10%, 500 mL), brine (500 mL*2), concentrated under reduced pressure to give ethyl (2s,4r)-6-oxo-5-azaspiro[3.4]octane-2-carboxylate (25.0 g, 127 mmol) as a yellow solid. ¹H NMR: (400 MHz, DMSO-d₆) δ 8.15 (s, 1H), 4.10-4.02 (m, 2H), 3.04-2.99 (m, 1H), 2.41-2.32 (m, 4H), 2.11-2.09 (m, 2H), 2.04-1.91 (m, 2H), 1.20-1.16 (m, 3H).

### Step 5. Synthesis of (2s,4r)-6-oxo-5-azaspiro[3.4]octane-2-carboxylic acid

To a mixture of compound ethyl (2s,4r)-6-oxo-5-azaspiro[3.4]octane-2-carboxylate (25.0 g, 127 mmol) in MeOH (225 mL) was added a solution of NaOH (15.2 g, 380 mmol) in H₂O (75.0 mL). The mixture was stirred at 20° C. for 12 hours. The mixture's pH was adjusted to 4 with HCl (4 N). The solution was concentrated to remove MeOH, then the mixture was filtered and the filter cake was dried over vacuum (part 1). The filtrate was concentrated. To the residue was added MeOH (100 mL) and the suspension was filtered. The filtrate was concentrated and the residue was purified by prep-HPLC (column: Phenomenex Luna C18 (250*80 mm*15 μm); mobile phase A: water; mobile phase B: acetonitrile; gradient: 1%-20% B over 20 minutes) to give more product (part 2). Part 1 and part 2 were combined and to give (2s,4r)-6-oxo-5-azaspiro[3.4]octane-2-carboxylic acid (15.5 g, 91.4 mmol) as a yellow amorphous solid. ¹H NMR: (400 MHz, DMSO-d₆) δ 12.17 (s, 1H), 7.98 (s, 1H), 2.79-2.70 (m, 1H), 2.29-2.20 (m, 4H), 2.14-2.08 (m, 4H).

### Example 9

### (2r,4r)-6-oxo-7-oxa-5-azaspiro[3.4]octane-2-carboxylic acid

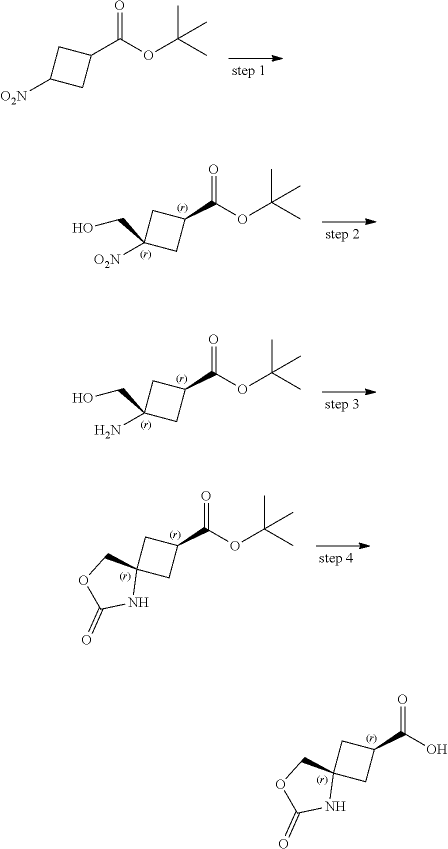 [SMILES: CC(C)(C)OC(=O)C1CC([N+](=O)[O-])C1 ; CC(C)(C)OC(=O)[C@H]1C[C@](CO)([N+](=O)[O-])C1 ; CC(C)(C)OC(=O)[C@H]1C[C@@](N)(CO)C1 ; CC(C)(C)OC(=O)[C@H]1C[C@@]2(COC(=O)N2)C1 ; O=C1N[C@]2(CO1)C[C@@H](C(=O)O)C2]

### Step 1. Synthesis of tert-butyl (1r,3r)-3-(hydroxymethyl)-3-nitrocyclobutane-1-carboxylate

To a solution of tert-butyl 3-nitrocyclobutane-1-carboxylate (81.0 g, 403 mmol) in acetonitrile (810 mL) was added (HCHO)n (48.6 g) at 25° C. To the mixture was added TEA (57.2 mL, 411 mmol) dropwise at 0° C. The mixture was stirred at 25° C. for 12 hours. The mixture was poured into water (2.00 L) and extracted with ethyl acetate (1.00 L*3). The combined organic layer was washed with brine (1.00 L), dried over Na₂SO₄, filtered and concentrated. The residue was purified by column chromatography (silica gel, petroleum ether/ethyl acetate 1/0 to 10/1) to give tert-butyl (1r,3r)-3-(hydroxymethyl)-3-nitrocyclobutane-1-carboxylate (22.4 g, 96.8 mmol) as a white solid. The other isomer is tert-butyl (1s,3s)-3-(hydroxymethyl)-3-nitrocyclobutane-1-carboxylate (49.2 g, 213 mmol).

tert-butyl (1r,3r)-3-(hydroxymethyl)-3-nitrocyclobutane-1-carboxylate ¹H NMR: (400 MHz, CDCl₃) δ 4.10 (d, J=6.4 Hz, 2H), 3.27-3.16 (m, 1H), 3.03-2.93 (m, 2H), 2.68-2.58 (m, 2H), 2.26 (t, J=6.6 Hz, 1H), 1.47 (s, 9H).

tert-butyl (1s,3s)-3-(hydroxymethyl)-3-nitrocyclobutane-1-carboxylate ¹H NMR: (400 MHz, CDCl₃) δ 4.03 (d, J=6.4 Hz, 2H), 3.00-2.91 (m, 2H), 2.88-2.78 (m, 1H), 2.64-2.56 (m, 2H), 2.41 (t, J=6.6 Hz, 1H), 1.46 (s, 9H).

### Step 2. Synthesis of tert-butyl (1r,3r)-3-amino-3-(hydroxymethyl)cyclobutane-1-carboxylate

To a mixture of tert-butyl (1r,3r)-3-(hydroxymethyl)-3-nitrocyclobutane-1-carboxylate (38.5 g, 166 mmol) in isopropanol (400 mL) was added Raney Ni (8.00 g) under N₂ at 25° C. The mixture was degassed under vacuum and purged with H₂ 3 times. The mixture was heated to 70° C. and stirred at 70° C. under H₂ (50 psi) for 12 hours. The mixture was filtered and the filtrate was concentrated to give tert-butyl (1r,3r)-3-amino-3-(hydroxymethyl)cyclobutane-1-carboxylate (33.0 g, crude) as an off-white solid. ¹H NMR: (400 MHz, CDCl₃) δ 3.47 (s, 2H), 3.13-2.98 (m, 1H), 2.32-2.27 (m, 2H), 2.09-1.96 (m, 2H), 1.43 (s, 9H).

### Step 3. Synthesis of tert-butyl (2r,4r)-6-oxo-7-oxa-5-azaspiro[3.4]octane-2-carboxylate

To a solution of compound tert-butyl (1r,3r)-3-amino-3-(hydroxymethyl)cyclobutane-1-carboxylate (33.0 g, 164 mmol) in THF (330 mL) was added TEA (48.7 mL, 350 mmol) at 25° C. To the mixture was added a solution of triphosgene (17.3 g, 58.4 mmol) in THF (120 mL) dropwise at −10° C. and the mixture was stirred at −10° C. for 0.5 hour. The reaction mixture was warmed to 25° C. and stirred for 2 hours. The mixture was poured into cold water (1.50 L) and extracted with ethyl acetate (1.00 L*3). The combined organic layer was washed with brine (1.00 L), dried over Na₂SO₄, filtered and concentrated. The residue was purified by column chromatography (silica gel, petroleum ether/ethyl acetate 1/0 to 5/1) to give tert-butyl (2r,4r)-6-oxo-7-oxa-5-azaspiro[3.4]octane-2-carboxylate (27.6 g, 121 mmol) as a light yellow solid. ¹H NMR: (400 MHz, CDCl₃) δ 6.78 (s, 1H), 4.47 (s, 2H), 2.90-2.84 (m, 1H), 2.66-2.56 (m, 2H), 2.51-2.44 (m, 2H), 1.46 (s, 9H).

### Step 4. Synthesis of (2r,4r)-6-oxo-7-oxa-5-azaspiro[3.4]octane-2-carboxylic acid

To tert-butyl (2r,4r)-6-oxo-7-oxa-5-azaspiro[3.4]octane-2-carboxylate (25.6 g, 113 mmol) was added TFA (250 mL, 3.38 mol) at 0° C. The mixture was stirred at 25° C. for 6 hours. The reaction mixture was concentrated. The residue was triturated with petroleum ether/ethyl acetate 1/1 (100 mL) at 25° C. for 0.5 hour. The mixture was filtered and the filter cake was dried under vacuum. To the solid was added water (250 mL) and it was lyophilized to give (2r,4r)-6-oxo-7-oxa-5-azaspiro[3.4]octane-2-carboxylic acid (18.0 g, 99.6 mmol) an off-white amorphous solid. ¹H NMR: (400 MHz, DMSO-d₆) δ 12.27 (br s, 1H), 8.21 (s, 1H), 4.28 (s, 2H), 2.94-2.80 (m, 1H), 2.48-2.36 (m, 4H).

### Example 10

### (2r,4S)—N—((S)-(3-chlorophenyl)(cyclopentyl)methyl)-5-oxo-6-azaspiro[3.4]octane-2-carboxamide and (2s,4R)—N—((S)-(3-chlorophenyl)(cyclopentyl)methyl)-5-oxo-6-azaspiro[3.4]octane-2-carboxamide

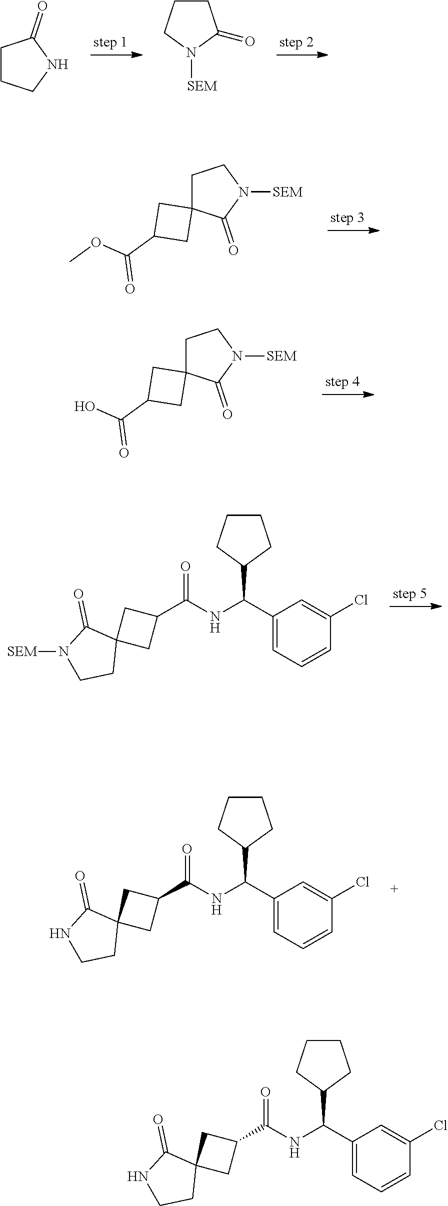

### Step 1. Synthesis of 1-((2-(trimethylsilyl)ethoxy)methyl)pyrrolidin-2-one

To a mixture of pyrrolidin-2-one (5 g, 0.059 mol) in THF (100 mL) was added NaH (1.69 g, 0.07 mol) in portions at 0° C. under a nitrogen atmosphere. The mixture was stirred for 1 h at 0° C. prior to the addition of (2-(chloromethoxy)ethyl)trimethylsilane (11.8 g, 0.07 mol) dropwise at 0° C. The mixture was stirred for 1 h at room temperature. The reaction was quenched with saturated NH₄Cl (aq.) and the aqueous phase was extracted with ethyl acetate (150 mL) three times. The combined organic layers were washed with brine, dried over sodium sulfate, filtered and concentrated in vacuo. The resulting crude material was purified by silica gel chromatography (100 g column; eluting with petroleum ether:ethyl acetate 5:1) to give 1-((2-(trimethylsilyl) ethoxy)methyl) pyrrolidin-2-one (4.0 g, 0.019 mol) as a yellow oil. ¹H NMR (400 MHz, DMSO-d6) δ 4.59 (s, 2H), 3.50-3.34 (m, 4H), 2.28 (t, J=8.0 Hz, 2H), 2.04-1.82 (m, 2H), 0.92-0.79 (m, 2H), 0.00 (s, 9H).

### Step 2. Synthesis of methyl 5-oxo-6-((2-(trimethylsilyl)ethoxy)methyl)-6-azaspiro[3.4]octane-2-carboxylate

To a mixture of 1-((2-(trimethylsilyl) ethoxy)methyl) pyrrolidin-2-one (5.0 g, 0.023 mol) in THF (100 mL) was added LDA (24.4 mL, 2 M in THF, 0.049 mol) dropwise at −78° C. under a nitrogen atmosphere. The mixture was stirred for 1 h at −78° C. prior to the addition of methyl 3-bromo-2-(bromomethyl) propanoate (6.0 g, 0.023 mol) dropwise at −78° C. The mixture was then stirred for 1 h at room temperature. The reaction was quenched with saturated NH₄Cl (aq.) and the aqueous phase was extracted with ethyl acetate (100 mL) three times. The combined organic layers were washed with brine, dried over sodium sulfate, filtered and concentrated in vacuo. The residue was purified by reverse phase flash chromatography (column: C18 silica gel; mobile phase A: water, B: Acetonitrile; gradient: 30% to 80% B in 10 min; detector: UV 220 nm to afford methyl 5-oxo-6-((2-(trimethylsilyl) ethoxy)methyl)-6-azaspiro [3.4]octane-2-carboxylate (200 mg, 0.64 mmol). LCMS RT 1.202 min, [M+H]⁺ 314.2, LCMS method C.

### Step 3. Synthesis of 5-oxo-6-((2-(trimethylsilyl)ethoxy)methyl)-6-azaspiro[3.4]octane-2-carboxylic acid

A mixture of methyl 5-oxo-6-((2-(trimethylsilyl) ethoxy)methyl)-6-azaspiro[3.4]octane-2-carboxylate (190 mg, 0.61 mmol) and NaOH (72.7 mg, 1.82 mmol) in MeOH/H₂O (1:1, 3 mL) was stirred for 1 h at room temperature. Concentration in vacuo gave the sodium salt of 5-oxo-6-((2-(trimethylsilyl)ethoxy)methyl)-6-azaspiro[3.4]octane-2-carboxylic acid (160 mg, 0.50 mmol) as a yellow oil. LCMS RT 0.655 min, [M+H]⁺ 300.2, LCMS method B.

### Step 4. Synthesis of (S)—N-((3-chlorophenyl)(cyclopentyl)methyl)-5-oxo-6-((2-(trimethylsilyl)ethoxy)methyl)-6-azaspiro[3.4]octane-2-carboxamide

A mixture of 5-oxo-6-((2-(trimethylsilyl) ethoxy)methyl)-6-azaspiro [3.4]octane-2-carboxylic acid (150 mg, 501 μmol), (S)-(3-chlorophenyl) (cyclopentyl)methanamine (105 mg, 501 μmol), DIEA (194 mg, 1.50 mmol) and HATU (381 mg, 1.00 mmol) in DMF (3 mL) was stirred for 1 h at room temperature. The reaction mixture was diluted with water (20 mL), and the aqueous phase was extracted with ethyl acetate (30 mL) three times. The combined organic layers were washed with brine, dried over sodium sulfate, filtered and concentrated in vacuo. The resulting crude material was purified by preparative HPLC (column: Xselect CSH C18 OBD Column 30*150 mm, 5 μm; mobile phase A: water (0.05% TFA), mobile phase B: acetonitrile; flow rate: 60 mL/min; gradient: 66% B to 75% B in 9 min, then 75% B; wavelength: 254/220 nm; RT: 7.15 min) to give (S)—N-((3-chlorophenyl) (cyclopentyl)methyl)-5-oxo-6-((2-(trimethylsilyl) ethoxy)methyl)-6-azaspiro [3.4]octane-2-carboxamide (70 mg, 0.14 mmol) as an off-white amorphous solid. LCMS RT 1.402 min, [M+H]⁺ 491.40, LCMS method B.

### Step 5. Synthesis of (2r,4S)—N—((S)-(3-chlorophenyl)(cyclopentyl)methyl)-5-oxo-6-azaspiro[3.4]octane-2-carboxamide and (2s,4R)—N—((S)-(3-chlorophenyl)(cyclopentyl)methyl)-5-oxo-6-azaspiro[3.4]octane-2-carboxamide

A mixture of (S)—N-((3-chlorophenyl) (cyclopentyl)methyl)-5-oxo-6-((2-(trimethylsilyl) ethoxy)methyl)-6-azaspiro [3.4]octane-2-carboxamide (50 mg, 0.10 mmol) in TFA (2 mL) was stirred for 1 h at room temperature. The mixture was concentrated in vacuo. Then the residue was dissolved in MeOH (1 mL). Ethylenediamine (61 mg, 1.0 mmol) was added, and the solution was stirred for 2 h at 80° C. After concentration in vacuo, the resulting crude material was purified by preparative HPLC (column: CHIRALPAK IC, 2*25 cm, 5 μm; mobile phase A: hexane, mobile phase B: EtOH; flow rate: 20 mL/min; gradient: 20% B; wavelength: 220/254 nm; RT1 (min): 9.15; RT2 (min): 14.23; sample solvent: EtOH; injection volume: 1.35 mL), then further purified by chiral preparative HPLC (column: DZ-CHIRALPAK ID-3, 4.6*50 mm, 3.0 μm; mobile phase: Hexane:EtOH 80:20; flow rate: 1 mL/min; gradient: isocratic; injection volume: 5 mL) to give one isomer (1 mg, 3 μmol) as an off-white amorphous solid. LCMS RT 1.496 min, [M+H]⁺ 361.2, LCMS method F; 1H NMR (400 MHz, DMSO) δ 1.08 (dq, J=17.0, 8.1 Hz, 1H), 1.27 (d, J=24.3 Hz, 3H), 1.37-1.67 (m, 5H), 1.80 (s, 1H), 1.95-2.07 (m, 2H), 2.31 (p, J=8.8 Hz, 2H), 2.50 (s, 1H), 2.52 (s, 1H), 2.74 (dd, J=14.4, 3.8 Hz, 1H), 3.04-3.17 (m, 2H), 4.58 (dd, J=10.5, 8.6 Hz, 1H), 5.36 (s, 1H), 5.59 (s, 1H), 7.24-7.38 (m, 3H), 7.45 (d, J=1.9 Hz, 1H), 7.61 (s, 1H), 8.49 (d, J=8.7 Hz, 1H). The other isomer (1 mg, 3 μmol) was also obtained as an off-white amorphous solid. LCMS RT 1.497 min, [M+H]⁺ 361.2, LCMS method F; 1H NMR (400 MHz, DMSO) δ 1.10 (dd, J=12.5, 8.3 Hz, 1H), 1.27 (dt, J=20.7, 6.5 Hz, 2H), 1.37-1.76 (m, 5H), 1.83 (s, 1H), 2.01 (td, J=15.5, 14.9, 10.7 Hz, 2H), 2.13-2.4 (m, 2H), 2.50 (s, 1H), 2.52 (s, 1H), 2.73 (dd, J=14.4, 3.8 Hz, 1H), 3.01-3.17 (m, 2H), 4.55 (dd, J=10.5, 8.6 Hz, 1H), 5.35 (s, 1H), 5.61 (s, 1H), 7.23-7.38 (m, 3H), 7.44 (d, J=1.7 Hz, 1H), 7.60 (s, 1H), 8.50 (d, J=8.6 Hz, 1H).

### Example 11

### N—((R)-(3-chlorophenyl)(cyclopentyl)methyl)-7-fluoro-6-oxo-5-azaspiro[3.4]octane-2-carboxamide

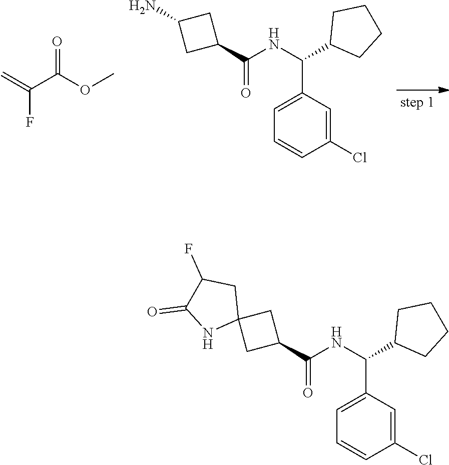

### Step 1. Synthesis of N—((R)-(3-chlorophenyl)(cyclopentyl)methyl)-7-fluoro-6-oxo-5-azaspiro[3.4]octane-2-carboxamide

To a 4-mL vial there was added (1r,3R)-3-amino-N—((R)-(3-chlorophenyl)(cyclopentyl)methyl)cyclobutane-1-carboxamide (50 mg, 0.16 mmol), tetrabutylammonium azide (4.6 mg, 16 μmol), 4CzIPN (1.3 mg, 1.6 μmol), and Cs₂CO₃ (53 mg, 0.16 mmol). The vial was capped and purged with nitrogen. Acetonitrile (1.1 mL) was added. The vial was sparged with nitrogen and while sparging, methyl 2-fluoroacrylate (15 μL, 0.16 mmol) was added via syringe. The reaction was then placed in the Merch photoreactor for 16 hours at 100% light intensity. The solution was concentrated and placed on the AccQ prep system eluting with 30-60% water with 0.1% formic acid to give N—((R)-(3-chlorophenyl)(cyclopentyl)methyl)-7-fluoro-6-oxo-5-azaspiro[3.4]octane-2-carboxamide (3.3 mg, 8.7 μmol) as an off-white solid. LCMS RT 1.44 min, [M+H]+379.23, LCMS method K.

### Example 12

### (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide

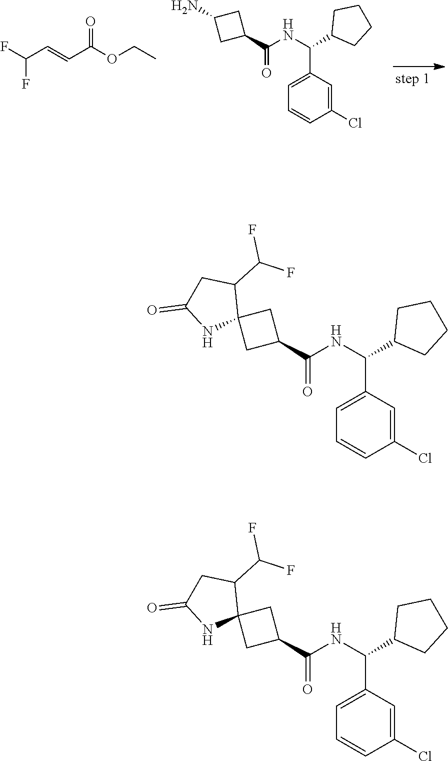

### Step 1. Synthesis of (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide

To a 8-mL vial there was added (1r,3R)-3-amino-N—((R)-(3-chlorophenyl)(cyclopentyl)methyl)cyclobutane-1-carboxamide (150 mg, 489 μmol) which was stirred with Cs₂CO₃ (159 mg, 489 μmol) in MeOH for 1 hour before filtering off the Cs₂CO₃ to convert the material to the free base. Tetrabutylammonium azide (13.9 mg, 48.9 μmol) and 4CzIPN (3.86 mg, 4.89 μmol) were added. The vial was capped and purged with nitrogen and dissolved in acetonitrile (2 mL). The vial was sparged with nitrogen and while sparging ethyl (E)-4,4-difluorobut-2-enoate (66.5 μL, 489 μmol) was added via syringe. The reaction was then placed in the Merch photoreactor for 8 hours at 100% light intensity. Reaction was concentrated and the vial was placed on the AccQ prep system, eluting with 20-50% water with 0.1% formic acid to give (2r,4R)—N—((R)-(3-chlorophenyl)(cyclopentyl)methyl)-8-(difluoromethyl)-6-oxo-5-azaspiro[3.4]octane-2-carboxamide and (2s,4S)—N—((R)-(3-chlorophenyl)(cyclopentyl)methyl)-8-(difluoromethyl)-6-oxo-5-azaspiro[3.4]octane-2-carboxamide, both as an off-white solid. Peak 1: 4.2 mg, LCMS RT 1.51 min, [M+H]⁺ 411.34, LCMS method K.

Peak 2: 5 mg, LCMS RT 1.53 min, [M+H]⁺ 411.34, LCMS method K.

### Example 13

### N-((1S,2R,4S)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide

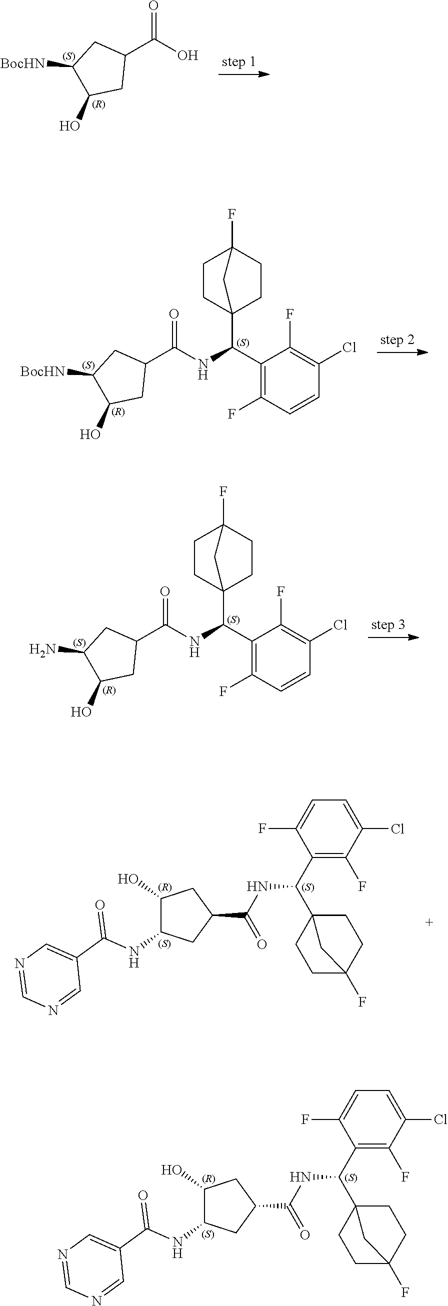

### Step 1. Synthesis of (3S,4R)-3-((tert-butoxycarbonyl)amino)-4-hydroxycyclopentane-1-carboxylic acid

A round bottomed flask was charged with (3S,4R)-3-((tert-butoxycarbonyl)amino)-4-hydroxycyclopentane-1-carboxylic acid (110 mg, 449 μmol), (S)-(3-chloro-2,6-difluorophe nyl)(4-fluorobicyclo[2.2.1]heptan-1-yl)methanamine (130 mg, 449 μmol), T3P (256 mg, 67 3 μmol), TEA (113 mg, 1.35 mmol) and a stirbar. DMF (1 mL) was added, and the solution was stirred for 1 h at 25° C. The reaction mixture was diluted with water (50 mL), and the a queous phase was extracted with ethyl acetate three times. The combined organic layers wer e washed with brine, dried over sodium sulfate, filtered and concentrated in vacuo. The resid ue was purified by reverse phase flash chromatography with the following condition: colum n: C18 silica gel; mobile phase A: water, mobile phase B: acetonitrile; gradient: 0% to 100% B in 10 min; detector: UV 220 nm. Lyophilization yielded tert-butyl ((1S,2R)-4-(((S)-(3-chloro-2,6-difluorophenyl) (4-fluorobicyclo[2.2.1]heptan-1-yl)methyl)carbamoyl)-2-hydroxycy clopentyl)carbamate (150 mg, 290 μmol) as an amorphous off-white solid. LCMS RT 1.094 min, [M+H]⁺ 517, LCMS method D.

### Step 2. Synthesis of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide

A round bottomed flask was charged with tert-butyl ((1S,2R)-4-(((S)-(3-chloro-2,6-difluorophenyl)(4-fluorobicyclo[2.2.1]heptan-1-yl)methyl)carbamoyl)-2-hydroxycyclopentyl)carbamate (1.5 g, 2.9 mmol) and a stirbar. HCl (15 mL, 4 molar in MeOH, 60 mmol) was added, and the solution was stirred for 30 minutes at 25° C. Concentration in vacuo resulted in (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (1.0 g, 2 mmol, crude) as a white solid. No workup was performed. LCMS RT 0.898 min, [M+H]⁺ 417.25, LCMS method D.

### Step 3. Synthesis of N-((1S,2R,4S)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-hydroxycyclopentyl)pyrimidine-5-carboxamide

A mixture of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (350 mg, 840 μmol), pyr imidine-5-carboxylic acid (104 mg, 840 μmol), NaHCO₃ (212 mg, 2.52 mmol) and HATU (638 mg, 1.68 mmol) in DMF (5 mL) was stirred for 1 hour at 25° C. The reaction mixture w as diluted with water (10 mL), and the aqueous phase was extracted with ethyl acetate (10 m L) three times. The combined organic layers were washed with brine, dried over sodium sulfate, filtered and concentrated in vacuo. The residue was purified by reverse phase flash chro matography (column: C18 silica gel; mobile phase A: water; mobile phase B: acetonitrile. G radient: 40% to 60% B in 10 min; detector: UV 220 nm, which afforded N-((1S,2R)-4-(((S)-(3-chloro-2,6-difluorophenyl)(4-fluorobicyclo[2.2.1]heptan-1-yl)methyl)carbamoyl)-2-hydr oxycyclopentyl)pyrimidine-5-carboxamide (335 mg, 76.3%) as an off-white amorphous sol id. LCMS RT 0.842 min, [M+H]⁺523, LCMS method C.

The resulting material was purified by chiral preparative HPLC (column: CHIRALPAKIF3; mobile phase A: hexane (0.2% DEA); B: MeOH:DCM 1:1) gradient: 75:25 isocratic; flow rate: 1 mL/min; injection volume: 3 mL). Lyophilization yielded one isomer (12 mg, 23 μmol) as an off-white amorphous solid and the other isomer (15 mg, 29 μmol, 60%), also as an off-white amorphous solid. Peak 1: ¹H NMR (400 MHz, DMSO-d₆) δ 9.29 (d, J=1.2 Hz, 1H), 9.17 (d, J=1.1 Hz, 2H), 8.43 (d, J=7.5 Hz, 1H), 8.26 (d, J=8.2 Hz, 1H), 7.57 (td, J=8.7, 5.4 Hz, 1H), 7.15 (t, J=9.1 Hz, 1H), 5.29 (d, J=8.1 Hz, 1H), 4.88 (d, J=3.4 Hz, 1H), 4.20-4.12 (m, 1H), 4.10 (d, J=3.9 Hz, 1H), 3.17 (dt, J=13.0, 6.4 Hz, 1H), 2.06-1.88 (m, 2H), 1.86-1.61 (d, J=8.4 Hz, 10H), 1.47 (d, J=8.3 Hz, 2H). LCMS RT 1.398 min, [M+H]⁺ 523, LCMS method D. Peak 2: ¹H NMR (400 MHz, DMSO-d₆) δ 9.28 (s, 1H), 9.15 (s, 2H), 8.42 (d, J=7.2 Hz, 1H), 8.26 (d, J=8.3 Hz, 1H), 7.57 (td, J=8.6, 5.4 Hz, 1H), 7.15 (td, J=9.5, 1.6 Hz, 1H), 5.28 (d, J=8.1 Hz, 1H), 4.88 (d, J=3.2 Hz, 1H), 4.19-4.08 (m, 2H), 3.21-3.13 (m, 1H), 1.96-1.68 (m, 11H), 1.61 (d, J=8.5 Hz, 1H), 1.47 (d, J=8.9 Hz, 2H). LCMS RT 1.404 min, [M+H]⁺ 523, LCMS method D.

### Example 14

### (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide

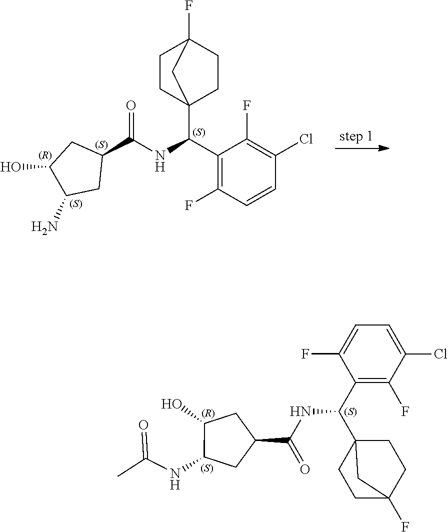

### Step 1. Synthesis of (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide

A round bottomed flask was charged with (1S,3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (1.45 g, 3.48 mmol), acetic acid (209 mg, 3.48 mmol), NaHCO₃ (1.46 g, 17.4 mmol), HATU (2.64 g, 6.96 mmol) and a stir bar. DMF (15 mL) was added, and the solution was stirred for 1 hour at room temperature. The resulting crude material was purified by preparative HPLC (column: LuxCellulose-34.6*100 mm, 3 μm; mobile phase A: water, mobile phase B: MeOH (0.5% 2 M NH₃ in MeOH); flow rate: 4 mL/min; gradient: 20% B isocratic) to give (1S,3S,4R)-3-acetamido-N—((S)-(3-chloro-2,6-difluorophenyl)(4-fluorobicyclo[2.2.1]heptan-1-yl)methyl)-4-hydroxycyclopentane-1-carboxamide (1.34 g, 2.93 mmol) as an off-white amorphous solid. ¹H NMR (400 MHz, DMSO-d₆) δ 8.22 (d, J=8.2 Hz, 1H), 7.61-7.48 (m, 2H), 7.19-7.09 (m, 1H), 5.26 (d, J=8.1 Hz, 1H), 4.78 (d, J=3.3 Hz, 1H), 3.96-3.85 (m, 2H), 3.15-3.03 (m, 1H), 1.82 (d, J=5.0 Hz, 2H), 1.81 (s, 3H), 1.75 (ddd, J=21.1, 11.5, 8.1 Hz, 8H), 1.59 (d, J=8.8 Hz, 2H), 1.45 (d, J=9.6 Hz, 2H). LCMS RT 0.833 min, [M+H]⁺ 459.05, LCMS method C.

### Example 15

### N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide

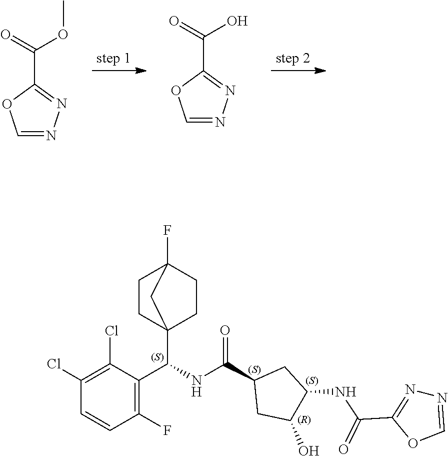

### Step 1. Synthesis of 1,3,4-oxadiazole-2-carboxylic acid

To a stirred mixture of methyl 1,3,4-oxadiazole-2-carboxylate (200 mg, 1.56 mmol) in THF (1 mL) and H₂O (1 mL) was added LiOH (74.8 mg, 3.12 mmol) at 25° C. under a nitrogen atmosphere. The resulting mixture was stirred for 1 hour at 25° C. under nitrogen. The mixture was acidified to pH 7 with HCl (aq. 1 M). The resulting mixture was concentrated under reduced pressure to afford 1,3,4-oxadiazole-2-carboxylic acid (260 mg, 2.28 mmol, crude) as a white solid. LCMS RT 0.177 min, [M−H]⁻ 113.0, LCMS method E.

### Step 2. Synthesis of N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide

To a stirred mixture of (1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide (150 mg, 346 μmol) and 1,3,4-oxadiazole-2-carboxylic acid (47.4 mg, 415 μmol) in DMF (2 mL) was added sodium bicarbonate (145 mg, 1.73 mmol) and HATU (395 mg, 1.04 mmol) at 25° C. under a nitrogen atmosphere. The resulting mixture was stirred for 2 hours at 25° C. The resulting mixture was filtered and purified by preparative HPLC (column: Xbridge Prep OBD C18 Column, 50*250 mm, 10 μm; mobile phase A: water (10 mM NH₄HCO₃+0.05% NH₄OH), mobile phase B: acetonitrile; flow rate: 100 mL/min; gradient: 25% B to 55% B in 8 min; wavelength: 254 nm/220 nm; RT (min): 9.58) to give a white solid. This was further purified by prep chiral HPLC (column: CHIRALPAK IG, 3*25 cm, 5 μm; mobile phase A: hexane:MTBE 1:1 (0.5% 2 M NH₃ in MeOH), mobile phase B: MeOH; flow rate: 40 mL/min; gradient: 20% B isocratic; wavelength: 212/230 nm; RT1 (min): 4.62; RT2 (min): 6.96; sample solvent: MeOH; injection volume: 0.9 mL) to give N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)-1,3,4-oxadiazole-2-carboxamide (14.6 mg, 26.9 μmol) as a white solid. LCMS RT 1.838 min, [M−H]⁻ 527.10, LCMS method E. ¹H NMR (300 MHz, DMSO-d6) δ 9.44 (s, 1H), 8.50 (d, J=6.8 Hz, 1H), 8.26 (d, J=8.2 Hz, 1H), 7.63 (dd, J=9.0, 5.1 Hz, 1H), 7.28 (dd, J=10.6, 8.9 Hz, 1H), 5.52 (d, J=8.0 Hz, 1H), 5.12 (d, J=3.0 Hz, 1H), 4.11 (s, 2H), 3.16 (t, J=7.1 Hz, 1H), 2.03-1.46 (m, 14H). ¹⁹F NMR (282 MHz, DMSO-d6) δ −109.304, −173.540.

### Example 16

### (1S,3S,4R)-3-((1R,2S)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide and (1S,3S,4R)-3-((1S,2R)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide

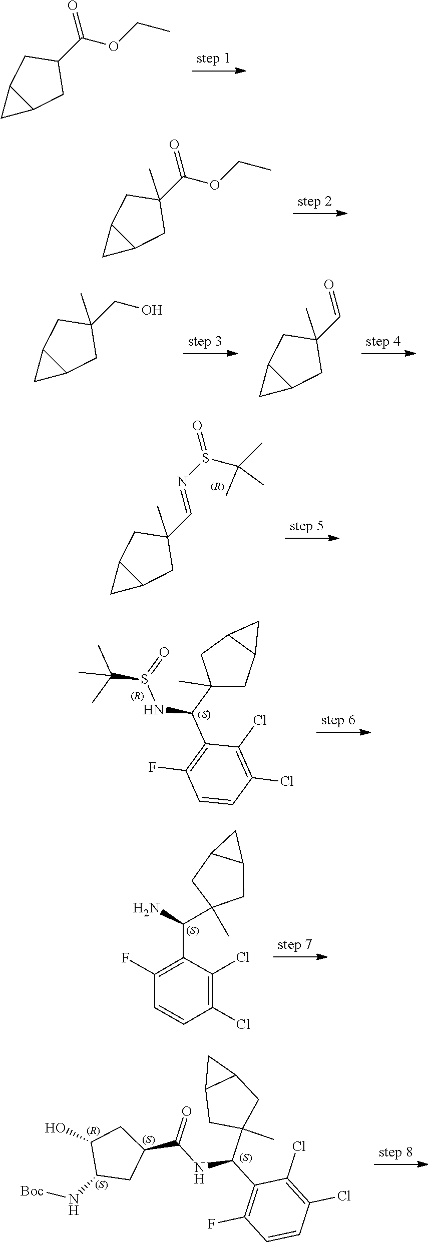

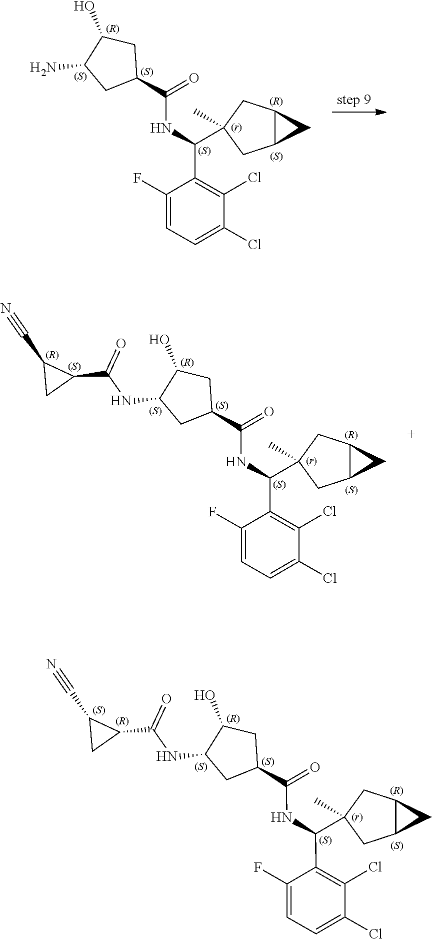

### Step 1. Synthesis of ethyl 3-methylbicyclo[3.1.0]hexane-3-carboxylate

To a mixture of ethyl bicycle [3.1.0]hexane-3-carboxylate (9.0 g, 0.058 mol) in THF (120 mL) was added LDA (45 ml, 2 M in THF, 0.09 mol) dropwise at −78° C. under a nitrogen atmosphere. The mixture was stirred for 1 h at −78° C. prior to addition of Mel (5 mL, 0.09 mol). The mixture was stirred for 2 h at 25° C. The reaction was quenched with saturated NH₄Cl (aq., 30 ml). The reaction mixture was diluted with water (100 mL), and the aqueous phase was extracted with ethyl acetate (100 mL*3). The combined organic layers were washed with brine, dried over sodium sulfate, filtered and concentrated in vacuo to give ethyl 3-methylbicyclo[3.1.0]hexane-3-carboxylate (9.0 g, 0.053 mol) as a yellow oil. GCMS RT 4.141 min, [M] 168.1, GC Method Z.

### Step 2. Synthesis of (3-methylbicyclo[3.1.0]hexan-3-yl)methanol

To a mixture of ethyl 3-methylbicyclo[3.1.0]hexane-3-carboxylate (10 g, 59 mmol) in THF (120 mL) was added LiAlH₄ (2.3 g, 59 mmol) in portions at 0° C. under a nitrogen atmosphere. The mixture was stirred for 2 hours at 25° C. The reaction was quenched with water (2.3 mL), NaOH (15%, 4.6 mL) and water (2.3 mL). The reaction mixture was filtered through a pad of Celite. The pad was washed with THF (100 mL), and the filtrate was concentrated in vacuo to give (3-methylbicyclo[3.1.0]hexan-3-yl) methanol (7.0 g) as a yellow oil. GCMS RT 3.760 min, [M]126.0, GC Method Z.

### Step 3. Synthesis of 3-methylbicyclo[3.1.0]hexane-3-carbaldehyde

To a mixture of (3-methylbicyclo[3.1.0]hexan-3-yl) methanol (7.0 g, 55.47 mmol) in DCM (90 mL) was added PCC (13.15 g, 61.01 mmol) in portions at 0° C. under a nitrogen atmosphere. The mixture was stirred for 2 hours at 25° C. The reaction mixture was filtered (through pad of silica gel), the pad was washed with DCM. The filtrate was concentrated under reduced pressure to afford 3-methylbicyclo[3.1.0]hexane-3-carbaldehyde (6.0 g) as a brown oil. GCMS RT 3.484 min, [M]124.1, GC Method Z.

### Step 4. Synthesis of (R)-2-methyl-N—((E)-(3-methylbicyclo[3.1.0]hexan-3-yl)methylene)propane-2-sulfinamide

To a solution of (R)-2-methylpropane-2-sulfinamide (6000 mg, 1 Eq, 49.50 mmol) and 3-methylbicyclo[3.1.0]hexane-3-carbaldehyde (6.762 g, 1.1 Eq, 54.46 mmol) in THF (75 mL) was added titanium(IV) isopropoxide (15.48 g, 16.5 mL, 1.1 Eq, 54.46 mmol). The mixture was heated at 50° C. for 16 hours. The reaction was quenched with water (100 mL). The reaction mixture was filtered (through a pad of Celite), the pad was washed with ethyl acetate (150 mL), and the filtrate was concentrated in vacuo. The residue was purified by reverse phase flash chromatography (column: C18 silica gel; mobile phase A: water, mobile phase B: acetonitrile; 10% to 50% gradient in 10 min; detector: UV 220 nm. This resulted in (R)-2-methyl-N—((E)-(3-methylbicyclo[3.1.0]hexan-3-yl)methylene) propane-2-sulfinamide (9.2 g, 40 mmol, 82%) as a white solid. LCMS RT 0.997 min, [M+H]⁺ 228.15, LCMS method C.

### Step 5. Synthesis of (R)—N-((1S)-(2,3-dichloro-6-fluorophenyl)(3-methylbicyclo[3.1.0]hexan-3-yl)methyl)-2-methylpropane-2-sulfinamide

To a mixture of 1,2-dichloro-4-fluorobenzene (1.742 g, 10.56 mmol) in THF (25 mL) was added LDA (6.6 ml, 2 M in THF, 13.2 mmol) dropwise at −78° C. under a nitrogen atmosphere. The mixture was stirred for 1 h at −78° C. prior to the addition of (R)-2-methyl-N—((E)-(3-methylbicyclo[3.1.0]hexan-3-yl)methylene) propane-2-sulfinamide (2.0 g, 8.796 mmol). The mixture was stirred for 2 h at 25° C. The reaction was quenched with saturated NH₄Cl (aq., 15 ml). The reaction mixture was diluted with water (20 mL), and the aqueous phase was extracted with ethyl acetate (50 mL*3). The combined organic layers were washed with brine, dried over sodium sulfate, filtered, and concentrated in vacuo. The residue was purified by reverse phase flash chromatography (column: C18 silica gel; mobile phase A: water, mobile phase B: acetonitrile; gradient: 10% to 50% B in 10 min; detector: UV 220 nm) to give (R)—N-((1S)-(2,3-dichloro-6-fluorophenyl) (3-methylbicyclo[3.1.0]hexan-3-yl)methyl)-2-methylpropane-2-sulfinamide (2.5 g, 6.4 mmol) as a yellow oil. LCMS RT 1.183 min, [M+H]⁺ 392, LCMS method A.

### Step 6. Synthesis of (1S)-(2,3-dichloro-6-fluorophenyl)(3-methylbicyclo[3.1.0]hexan-3-yl)methanamine

A mixture of (R)—N-((1S)-(2,3-dichloro-6-fluorophenyl) (3-methylbicyclo[3.1.0]hexan-3-yl)methyl)-2-methylpropane-2-sulfinamide (5.5 g, 14 mmol) and HCl (14 mL, 4 molar in MeOH, 56 mmol) was stirred for 1 h at 25° C. The mixture's pH was adjusted to 7-8 with saturated NaHCO₃ solution. The mixture was extracted with ethyl acetate (70 mL) three times. The combined organic layers were washed with brine, dried over sodium sulfate, filtered and concentrated in vacuo. This resulted in (1S)-(2,3-dichloro-6-fluorophenyl) (3-methylbicyclo[3.1.0]hexan-3-yl) methanamine (3.8 g, 13 mmol) as a yellow oil. LCMS RT 0.738 min, [M+H]⁺ 288.0, LCMS method C.

### Step 7. Synthesis of tert-butyl ((1S,2R,4S)-4-(((1S)-(2,3-dichloro-6-fluorophenyl)(3-methylbicyclo[3.1.0]hexan-3-yl)methyl)carbamoyl)-2-hydroxycyclopentyl)carbamate

A mixture of (1S)-(2,3-dichloro-6-fluorophenyl) (3-methylbicyclo[3.1.0]hexan-3-yl) methanamine (4 g, 0.01 mol), (1S,3S,4R)-3-((tert-butoxycarbonyl)amino)-4-hydroxycyclopentane-1-carboxylic acid (3 g, 0.01 mol), HATU (8 g) and NaHCO₃ (3 g) in DMF (40 mL) was stirred for 1 h at 25° C. The reaction mixture was diluted with water (50 mL), and the aqueous phase was extracted with ethyl acetate (60 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 220 nm) to give tert-butyl ((1S,2R,4S)-4-(((1S)-(2,3-dichloro-6-fluorophenyl) (3-methylbicyclo[3.1.0]hexan-3-yl)methyl) carbamoyl)-2-hydroxycyclopentyl) carbamate (5.1 g, 9.9 mmol) as an off-white solid. LCMS RT 1.080 min, [M+H]⁺ 515, LCMS method C.

### Step 8. Synthesis of (1S,3S,4R)-3-amino-N—((S)-(2,3-dichloro-6-fluorophenyl)((1R,3r,5S)-3-methylbicyclo[3.1.0]hexan-3-yl)methyl)-4-hydroxycyclopentane-1-carboxamide

A mixture of tert-butyl ((1S,2R,4S)-4-(((1S)-(2,3-dichloro-6-fluorophenyl) (3-methylbicyclo[3.1.0]hexan-3-yl)methyl) carbamoyl)-2-hydroxycyclopentyl) carbamate (2.0 g, 3.9 mmol) and HCl (19.40 mL, 4 N in MeOH, 77.60 mmol) in MeOH (20 mL) was stirred for 1 h at 25° C. The mixture's pH was adjusted to 7-8 with saturated NaHCO₃ solution. The reaction mixture was diluted with water (50 mL), and the aqueous phase was extracted with ethyl acetate (70 mL) three times. The combined organic layers were washed with brine, dried over sodium sulfate, filtered and concentrated in vacuo to give (1S,3S,4R)-3-amino-N-((1S)-(2,3-dichloro-6-fluorophenyl) (3-methylbicyclo[3.1.0]hexan-3-yl)methyl)-4-hydroxycyclopentane-1-carboxamide (1.5 g, 3.6 mmol) as a white amorphous solid. LCMS RT 0.780 min, [M+H]⁺ 415, LCMS method C.

### Step 9. Synthesis of (1S,3S,4R)-3-((1R,2S)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide and (1S,3S,4R)-3-((1S,2R)-2-cyanocyclopropane-1-carboxamido)-N—((S)-(2,3-dichloro-6-fluorophenyl)((1R,3r,5S)-3-methylbicyclo[3.1.0]hexan-3-yl)methyl)-4-hydroxycyclopentane-1-carboxamide

A mixture of (1S,3S,4R)-3-amino-N—((S)-(2,3-dichloro-6-fluorophenyl)((1S,3r,5R)-3-methylbicyclo[3.1.0]hexan-3-yl)methyl)-4-hydroxycyclopentanecarboxamide (45 mg, 0.11 mmol), (±)-(1S,2R)-2-cyanocyclopropane-1-carboxylic acid (12 mg, 0.11 mmol), HATU (62 mg, 0.16 mmol) and NaHCO₃ (36 mg, 0.43 mmol) in DMF (1 mL) was stirred at room temperature for 1 hour. The reaction mixture was diluted with water (10 mL), and the aqueous phase was extracted with ethyl acetate (10 mL*3). The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃)+0.05% NH₄OH, mobile phase B: acetonitrile; flow rate: 60 mL/min; gradient: 35% B to 62% B in 7 min; wavelength: 254 nm/220 nm; RT (min): 7.64) to give (1S,3S,4R)-3-((1S,2R)-2-cyanocyclopropane-1-carboxamido)-N—((S)-(2,3-dichloro-6-fluorophenyl) ((1R,3r,5S)-3-methylbicyclo[3.1.0]hexan-3-yl)methyl)-4-hydroxycyclopentane-1-carboxamide (51 mg, 0.10 mmol) as an off white amorphous solid. LCMS RT 1.118 min, [M+H]⁺ 508, LCMS method B. This material was further purified by chiral preparative HPLC(column: CHIRALPAK IE3; mobile phase A: hexane (0.2% diethylamine):(EtOH:DCM 1:1) 60:40; flow rate: 1 mL/min; gradient: isocratic; injection volume: 8 mL) to give (1S,3S,4R)-3-((1S,2R)-2-cyanocyclopropane-1-carboxamido)-N—((S)-(2,3-dichloro-6-fluorophenyl) ((1R,3r,5S)-3-methylbicyclo[3.1.0]hexan-3-yl)methyl)-4-hydroxycyclopentane-1-carboxamide and (1S,3S,4R)-3-((1R,2S)-2-cyanocyclopropane-1-carboxamido)-N—((S)-(2,3-dichloro-6-fluorophenyl) ((1R,3r,5S)-3-methylbicyclo[3.1.0]hexan-3-yl)methyl)-4-hydroxycyclopentane-1-carboxamide, both as an off white amorphous solid. One isomer is 10.5 mg (20.3 μmol), and the other is 12.5 mg (24.0 μmol). Isomer 1: ¹H NMR (400 MHz, DMSO-d₆) δ 8.12 (d, J=7.8 Hz, 1H), 8.01 (d, J=8.7 Hz, 1H), 7.60 (dd, J=9.0, 5.0 Hz, 1H), 7.25 (dd, J=10.7, 8.9 Hz, 1H), 5.41 (d, J=8.6 Hz, 1H), 4.88 (d, J=3.5 Hz, 1H), 3.98 (dt, J=14.8, 4.6 Hz, 2H), 3.13 (dt, J=14.0, 6.9 Hz, 1H), 2.23 (td, J=7.9, 6.3 Hz, 1H), 2.02 (td, J=8.6, 6.6 Hz, 1H), 1.86-1.59 (m, 5H), 1.52 (dd, J=12.8, 5.8 Hz, 1H), 1.44-1.21 (m, 6H), 1.14-1.02 (m, 3H), 0.85 (td, J=7.9, 4.0 Hz, 1H), 0.11 (q, J=3.9 Hz, 1H). LCMS RT 1.096 min, [M+H]⁺ 508, LCMS method B; isomer 2: ¹H NMR (400 MHz, DMSO-d₆) 8.07 (d, J=8.0 Hz, 1H), 7.98 (d, J=8.7 Hz, 1H), 7.60 (dd, J=9.0, 5.0 Hz, 1H), 7.24 (t, J=9.8 Hz, 1H), 5.41 (d, J=8.6 Hz, 1H), 4.93 (d, J=3.5 Hz, 1H), 4.10-3.91 (m, 2H), 3.14 (d, J=9.0 Hz, 1H), 2.26 (q, J=7.5 Hz, 1H), 2.03 (q, J=7.9 Hz, 1H), 1.80 (q, J=10.6, 7.9 Hz, 2H), 1.74-1.58 (m, 3H), 1.57-1.45 (m, 1H), 1.44-1.18 (m, 6H), 1.07 (s, 3H), 0.85 (s, 1H), 0.10 (d, J=4.1 Hz, 1H). LCMS RT 1.115 min, [M+H]⁺ 508, LCMS method B.

### Example 17

### (1S,3R)-3-acetamido-N—((S)-(2,3-dichloro-6-fluorophenyl)(1-methylcyclopentyl)methyl)-1-methylcyclopentane-1-carboxamide, (1R,3R)-3-acetamido-N—((S)-(2,3-dichloro-6-fluorophenyl)(1-methylcyclopentyl)methyl)-1-methylcyclopentane-1-carboxamide, (1S,3S)-3-acetamido-N—((S)-(2,3-dichloro-6-fluorophenyl)(1-methylcyclopentyl)methyl)-1-methylcyclopentane-1-carboxamide and (1R,3S)-3-acetamido-N—((S)-(2,3-dichloro-6-fluorophenyl)(1-methylcyclopentyl)methyl)-1-methylcyclopentane-1-carboxamide

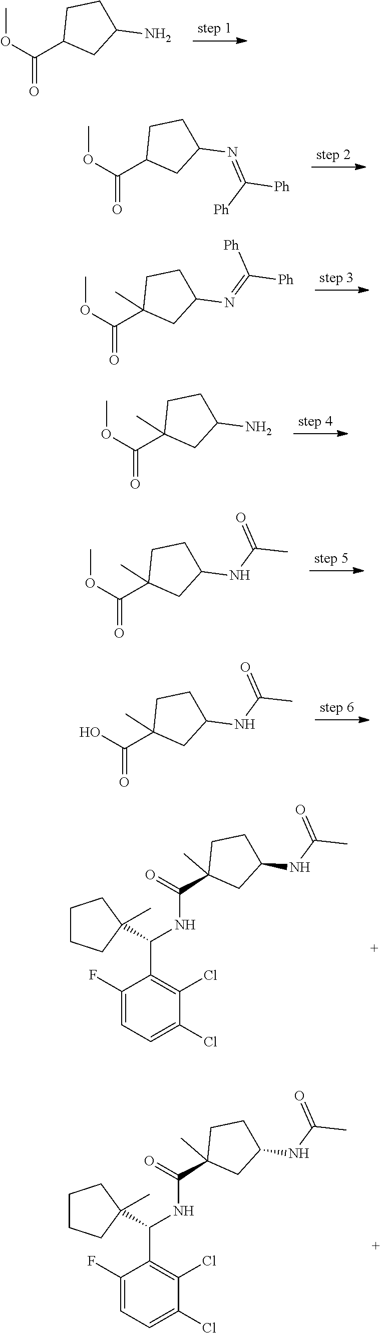

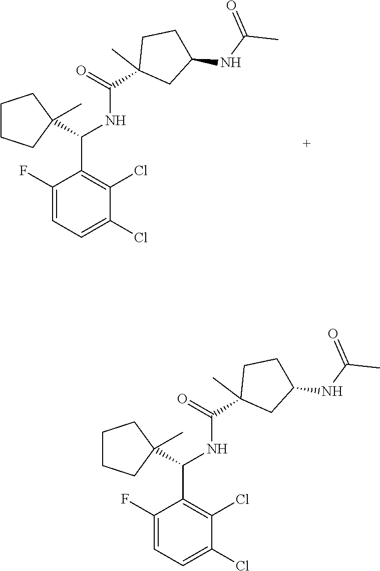 [SMILES: CC(=O)N[C@@H]1CC[C@@](C)(C(=O)N[C@H](c2c(F)ccc(Cl)c2Cl)C2(C)CCCC2)C1 ; CC(=O)N[C@H]1CC[C@@](C)(C(=O)N[C@H](c2c(F)ccc(Cl)c2Cl)C2(C)CCCC2)C1]

### Step 1. Synthesis of methyl 3-((diphenylmethylene)amino)cyclopentane-1-carboxylate

To a mixture of methyl 3-aminocyclopentane-1-carboxylate (4.6 g, 32 mmol) and T EA (18 mL, 0.13 mol) in DCM (50 mL) was added diphenylmethanimine (5.8 g, 32 mmol). The mixture was stirred at room temperature for 1 hour. The reaction mixture was filtered through a pad of Celite and the pad was washed with DCM (20 mL*3). The filtrate was conce ntrated in vacuo. The residue was purified by reverse phase flash chromatography (column: C18 silica gel; mobile phase A: water, mobile phase B: acetonitrile, gradient: 0% to 100% B in 20 min; detector: UV 254 nm) to give methyl 3-((diphenylmethylene)amino) cyclopenta ne-1-carboxylate (6.0 g) as a yellow oil. LCMS RT 0.670 min, [M+H]⁺ 308, LCMS method C.

### Step 2. Synthesis of methyl 3-((diphenylmethylene)amino)-1-methylcyclopentane-1-carboxylate

To a mixture of methyl 3-((diphenylmethylene)amino)cyclopentane-1-carboxylate (2.0 g, 6.51 mmol) in THF (30 mL) was added lithium diisopropylamide (3.9 mL, 2 molar, 7.8 mmol) dropwise at −78° C. under a nitrogen atmosphere. The mixture was stirred at −78° C. for 30 min prior to the addition of iodomethane (1.02 g, 7.16 mmol) dropwise at −78° C. The mixture was stirred at 25° C. for 1 hour. The reaction was quenched with saturated NH₄Cl (aq., 6 mL). The reaction mixture was diluted with water (40 mL), and the aqueous phase was extracted with ethyl acetate (50 mL*3). The combined organic layers were washed with brin e, dried over sodium sulfate, filtered and concentrated in vacuo. The residue was purified by reverse phase flash chromatography (column: C18 silica gel; mobile phase A: water, mobile phase B: acetonitrile; gradient: 0% to 100% in 20 min; detector: UV 254 nm) to give methyl 3-((diphenylmethylene)amino)-1-methylcyclopentane-1-carboxylate (1.5 g, 4.7 mmol) as a yellow oil. LCMS RT 0.712 min, [M+H]⁺ 322, LCMS method C.

### Step 3. Synthesis of methyl 3-amino-1-methylcyclopentane-1-carboxylate

A mixture of methyl 3-((diphenylmethylene)amino)-1-methylcyclopentane-1-carbox ylate (1.5 g, 4.7 mmol) in HCl (20 ml, 4 N) was stirred at 80° C. for 1 hour. The mixture was concentrated under reduced pressure to give methyl 3-amino-1-methylcyclopentane-1-carbo xylate (0.7 g, 4 mmol) as a yellow oil which was used in the next step directly without purifi cation. LCMS RT 0.479 min, [M+H]⁺ 158, LCMS method C.

### Step 4. Synthesis of methyl 3-acetamido-1-methylcyclopentane-1-carboxylate

To a mixture of methyl 3-amino-1-methylcyclopentane-1-carboxylate (700 mg, 4.45 mmol) and TEA (3.72 mL, 26.7 mmol) in DCM (10 mL) was added acetyl chloride (315 mg, 4.01 mmol) dropwise at 0° C. The mixture was stirred at room temperature for 1 hour. The reaction was quenched with MeOH (3 mL). The solution was concentrated under reduced pressure. The residue was purified by reverse phase flash chromatography (column: C18 silica gel; mobile phase A: water, mobile phase B: acetonitrile; gradient: 0% to 100% B in 15 min; detector: UV 254 nm) to give methyl 3-acetamido-1-methylcyclopentane-1-carboxylate (590 mg, 2.96 mmol) as a yellow oil. LCMS RT 0.612 min, [M+H]⁺ 200, LCMS method C.

### Step 5. Synthesis of 3-acetamido-1-methylcyclopentane-1-carboxylic acid

A mixture of methyl 3-acetamido-1-methylcyclopentane-1-carboxylate (590 mg, 2.9 6 mmol) and NaOH (5 mL, 4 N, aq.) in MeOH (5 mL) was stirred at room temperature for 1 hour. The solution was concentrated under reduced pressure. The mixture was acidified to p H of 4-6 with HCl (4 N). The solution was concentrated under reduced pressure. The residu e was purified by reverse phase flash chromatography (column: C18 silica gel; mobile phase A: water, mobile phase B: acetonitrile; gradient: 0% to 50% B in 10 min; detector: UV 254 nm) to give 3-acetamido-1-methylcyclopentane-1-carboxylic acid (510 mg, 2.75 mmol) as a yellow oil. LCMS RT 0.496 min, [M+H]⁺ 185, LCMS method C.

### Step 6. Synthesis of (1S,3R)-3-acetamido-N—((S)-(2,3-dichloro-6-fluorophenyl)(1-methylcyclopentyl)methyl)-1-methylcyclopentane-1-carboxamide, (1R,3R)-3-acetamido-N—((S)-(2,3-dichloro-6-fluorophenyl)(1-methylcyclopentyl)methyl)-1-methylcyclopentane-1-carboxamide, (1S,3S)-3-acetamido-N—((S)-(2,3-dichloro-6-fluorophenyl)(1-methylcyclopentyl)methyl)-1-methylcyclopentane-1-carboxamide and (1R,3S)-3-acetamido-N—((S)-(2,3-dichloro-6-fluorophenyl)(1-methylcyclopentyl)methyl)-1-methylcyclopentane-1-carboxamide

A mixture of (S)-(2,3-dichloro-6-fluorophenyl)(1-methylcyclopentyl)methanamine (500 mg, 1.81 mmol), 3-acetamido-1-methylcyclopentane-1-carboxylic acid (671 mg, 3.62 mmol), HATU (1.38 g, 3.62 mmol) and NaHCO₃ (0.61 g, 7.24 mmol) in DMF (5 mL) was stirred at room temperature for 1 hour. The reaction mixture was diluted with water (6 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 20 min; detector: UV 254 nm) to give 3-acetamido-N—((S)-(2,3-dichloro-6-fluorophenyl) (1-methylcyclopentyl)methyl)-1-methylcyclopentane-1-carboxamide (570 mg, 1.29 mmol) as a yellow oil. LCMS RT 1.146 min, [M+H]⁺ 443, LCMS method C.

The material was further purified by chiral preparative HPLC (column: (R, R)-WHELK-O1-Kromasi, 5*25 cm, 5 μm; mobile phase A: hexane (0.5% 2 M NH3-MeOH), mobile phase B: EtOH; flow rate: 20 mL/min; gradient: 35% B isocratic; wavelength: 220/254 nm; RT1 (min): 5.41; RT2 (min): 7.55; sample solvent: EtOH; injection volume: 0.4 mL) to give 3 peaks, then the peak that was still a mixture was purified by chiral preparative HPLC again (column: CHIRALPAK IH, 2*25 cm, 5 μm; mobile phase A: hexane (0.5% 2 M NH3-MeOH), mobile phase B: EtOH; flow rate: 20 mL/min; gradient: 50% B isocratic; wavelength: 220/254 nm; RT1 (min): 3.65; RT2 (min): 37.12; sample solvent: EtOH; injection volume: 2.65 mL) to give 4 compounds in total, all as a white amorphous solid. Product 1: 15 mg, 34 μmol. ¹H NMR (400 MHz, DMSO-d₆) δ 7.77 (d, J=7.2 Hz, 1H), 7.63 (dd, J=9.0, 5.1 Hz, 1H), 7.29 (dd, J=11.0, 8.9 Hz, 1H), 7.17 (d, J=8.8 Hz, 1H), 5.53 (d, J=8.6 Hz, 1H), 4.07 (h, J=7.4 Hz, 1H), 2.11-2.00 (m, 1H), 1.89 (dt, J=16.1, 7.1 Hz, 2H), 1.80 (dd, J=13.1, 8.3 Hz, 1H), 1.73 (s, 3H), 1.61 (s, 6H), 1.61-1.51 (m, 1H), 1.45-1.26 (m, 2H), 1.24 (s, 2H), 1.18 (s, 3H), 0.99 (d, J=2.9 Hz, 3H). LCMS RT 1.042 min, [M+H]⁺ 443, LC Method C. Product 2: 4.9 mg, 11 μmol. ¹H NMR (400 MHz, DMSO-d₆) δ 7.76 (d, J=7.4 Hz, 1H), 7.63 (dd, J=9.0, 5.1 Hz, 1H), 7.30 (dd, J=11.0, 9.0 Hz, 1H), 7.13 (d, J=9.0 Hz, 1H), 5.57 (d, J=8.8 Hz, 1H), 4.08 (h, J=7.6 Hz, 1H), 2.11 (ddd, J=12.6, 8.8, 6.0 Hz, 1H), 1.88 (ddd, J=24.6, 12.7, 6.9 Hz, 2H), 1.73 (s, 3H), 1.76-1.64 (m, 1H), 1.61 (s, 6H), 1.39 (ddt, J=36.1, 20.2, 7.5 Hz, 2H), 1.31 (s, 2H), 1.20 (s, 3H), 0.99 (d, J=2.9 Hz, 3H). LCMS RT 1.042 min, [M+H]⁺ 443, LCMS method C. Product 3: 80 mg, 0.18 mmol. ¹H NMR (400 MHz, DMSO-d₆) δ 7.82 (d, J=7.3 Hz, 1H), 7.62 (dd, J=9.0, 5.1 Hz, 1H), 7.32-7.19 (m, 2H), 5.51 (d, J=8.7 Hz, 1H), 3.92 (h, J=7.7 Hz, 1H), 2.34 (dd, J=13.2, 8.1 Hz, 1H), 2.00 (dt, J=12.6, 7.6 Hz, 1H), 1.84-1.71 (m, 1H), 1.75 (s, 3H), 1.60 (s, 6H), 1.61-1.49 (m, 1H), 1.42 (dq, J=15.3, 7.4 Hz, 2H), 1.30 (s, 1H), 1.25 (s, 3H), 1.21 (dd, J=13.1, 7.6 Hz, 1H), 0.99 (d, J=2.9 Hz, 3H). LCMS RT 1.452 min, [M+H]⁺ 443, LCMS method B. Product 4: 64 mg, 0.14 mmol. ¹H NMR (400 MHz, DMSO-d₆) δ 7.82 (d, J=7.3 Hz, 1H), 7.62 (dd, J=8.9, 5.0 Hz, 1H), 7.37-7.19 (m, 2H), 5.50 (d, J=8.5 Hz, 1H), 3.92 (p, J=7.6, 7.0 Hz, 1H), 2.40 (dd, J=13.2, 8.0 Hz, 1H), 1.96 (dt, J=12.4, 7.4 Hz, 1H), 1.75 (s, 3H), 1.78-1.67 (m, 1H), 1.61 (s, 6H), 1.56-1.34 (m, 3H), 1.25 (s, 3H), 1.33-1.18 (m, 2H), 0.99 (d, J=2.9 Hz, 3H). LCMS RT 1.425 min, [M+H]⁺ 443, LCMS method B.

### Example 18

### (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide

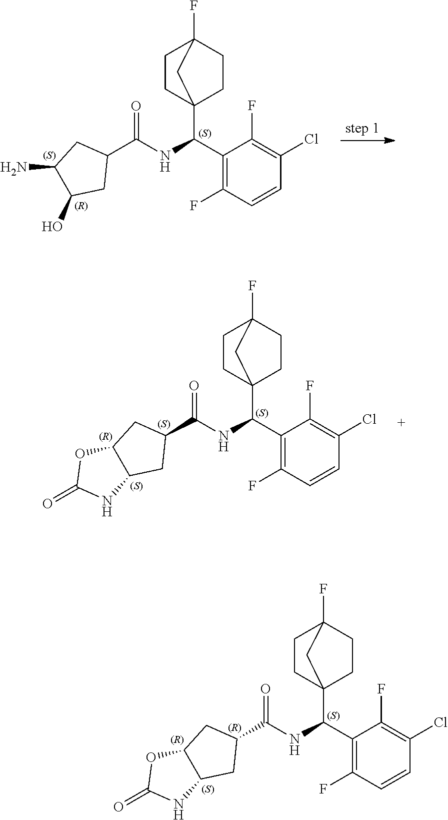

### Step 1. Synthesis of (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide

To a mixture of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (50 mg, 0.12 mmol) and pyridine (47 mg, 0.60 mmol) in DCM (5 mL) was added a solution of triphosgene (18 mg, 60 μmol) in DCM (0.5 mL) dropwise at 0° C. The mixture was stirred for 12 hours at 25° C. The reaction was quenched with saturated NH₄Cl (aq.). The reaction mixture was diluted with water (10 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give (3aS,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide (35 mg, 79 μmol) as a white amorphous solid. LCMS RT 0.951 min, [M+H]⁺ 443.1, LCMS method C.

The material was purified by prep chiral-HPLC (column: CHIRALPAK-IG3; mobile phase A: hexane (0.2% diethylamine), mobile phase B: EtOH:DCM 1:1, gradient: 40% B isocratic; flow rate: 1 mL/min; injection volume: 3 mL) to give (3aS,5S,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide and (3aS,5R,6aR)—N—((S)-(3-chloro-2,6-difluorophenyl)(4-fluorobicyclo[2.2.1]heptan-1-yl)methyl)-2-oxohexahydro-2H-cyclopenta[d]oxazole-5-carboxamide, both as a white amorphous solid. One isomer is 5.3 mg, 12 μmol. ¹HNMR (400 MHz, CDCl₃) δ 7.49-7.26 (m, 1H), 6.88 (t, J=9.5 Hz, 1H), 6.74 (d, J=9.7 Hz, 1H), 5.64 (d, J=9.8 Hz, 1H), 5.45 (s, 1H), 5.26-4.93 (m, 1H), 4.42 (s, 1H), 3.00 (d, J=12.9 Hz, 1H), 2.38-2.16 (m, 1H), 2.10-1.31 (m, 13H). LCMS RT 0.882 min, [M+H]⁺ 443.1, LCMS method C; the other isomer is 11.5 mg, 26.0 μmol. ¹H NMR (400 MHz, DMSO-d6) 8.48 (d, J=8.5 Hz, 1H), 7.57 (td, J=8.6, 5.4 Hz, 2H), 7.32-7.00 (m, 1H), 5.27 (d, J=8.1 Hz, 1H), 5.14-4.81 (m, 1H), 4.19 (t, J=6.5 Hz, 1H), 3.11 (tt, J=12.0, 6.1 Hz, 1H), 2.09-1.87 (m, 1H), 1.88-1.31 (m, 13H). LCMS RT 0.879 min, [M+H]⁺ 443.1, LCMS method C.

### Example 19

### (1S,2R,4S)-4-acetamido-N—((S)-(2,3-dichloro-6-fluorophenyl)(1-methylcyclopentyl)methyl)-2-(hydroxymethyl)cyclopentane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide

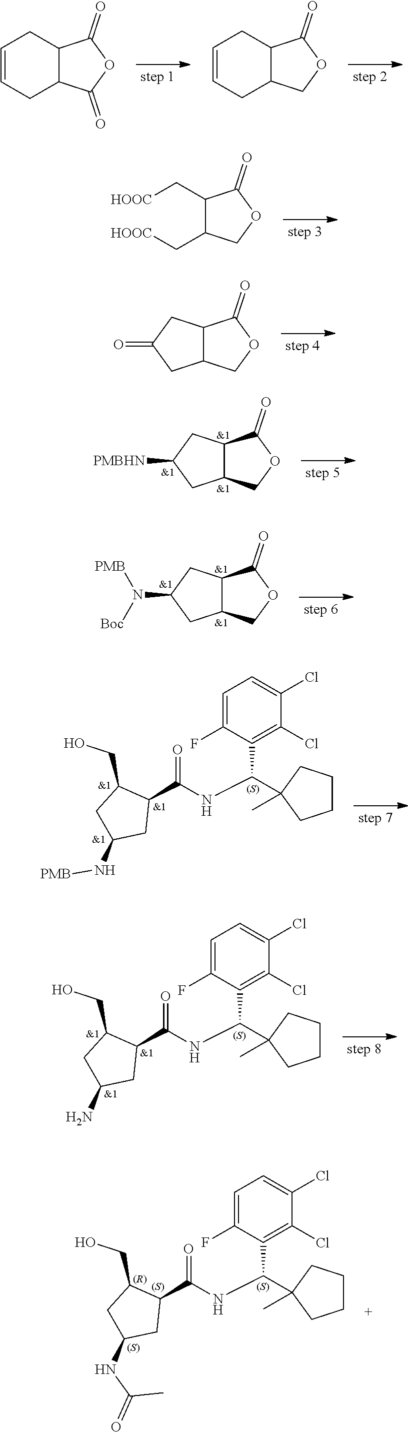

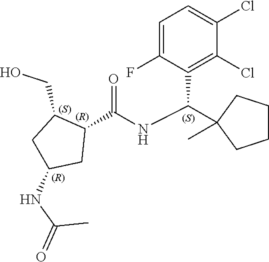

### Step 1. Synthesis of 3a,4,7,7a-tetrahydroisobenzofuran-1(3H)-one

To a mixture of 3a,4,7,7a-tetrahydroisobenzofuran-1,3-dione (20 g, 0.13 mol) in THF (200 mL) was added LiAlH₄ (5.0 g, 0.13 mol) in portions at 0° C. The mixture was stirred for 3 hours at room temperature. The resulting mixture was poured into 25 g of ice (mixed with 50 mL of 6% HCl in water) and extracted three times with ethyl acetate (200 ml*3). The combined organic layers were washed with brine and dried over anhydrous MgSO₄. The crude product was purified by silica gel chromatography (200 g column; eluting with petroleum ether/ethyl acetate; ratio: 10/1) to give 3a,4,7,7a-tetrahydroisobenzofuran-1(3H)-one (7 g, 0.05 mol) as a yellow oil. ¹H NMR (400 MHz, DMSO-d₆) δ 5.77-5.64 (m, 2H), 4.29 (dd, J=8.6, 4.9 Hz, 1H), 3.98 (dd, J=8.6, 1.5 Hz, 1H), 3.17 (d, J=5.2 Hz, 1H), 2.93 (td, J=7.3, 3.6 Hz, 1H), 2.64-2.52 (m, 1H), 2.44-2.01 (m, 3H).

### Step 2. Synthesis of 2,2′-(2-oxotetrahydrofuran-3,4-diyl)diacetic acid

To a mixture of KMnO₄ (15 g, 98 mmol) in H₂O (180 mL) was added a solution of 3a,4,7,7a-tetrahydroisobenzofuran-1(3H)-one (4.5 g, 33 mmol) in acetone (36 mL) dropwise at 0° C. The brown slurry was stirred for 1 h at 0° C., warmed to room temperature and stirre d overnight. The reaction was quenched with NaHSO₃. The resulting slurry was filtered thro ugh a pad of Celite and the Celite was washed with water/THF (1/1, 250 mL). The combine d filtrate was acidified to pH 2. The mixture was diluted with saturated NaCl (aq.) and extra cted with tert-butyl methyl ether/THF (⅔, 6×120 mL). The combined organic layers were dried over Na₂SO₄, filtered and concentrated under reduced pressure (the bath temperature not exceeding 30° C.) to give 2,2′-(2-oxotetrahydrofuran-3,4-diyl)diacetic acid (5.5 g, 27 mmol) as an off-white solid. LCMS RT 0.238 min, [M+H]⁺ 203.05. LCMS method B.

### Step 3. Synthesis of tetrahydro-1H-cyclopenta[c]furan-1,5(3H)-dione

A mixture of 2,2′-(2-oxotetrahydrofuran-3,4-diyl)diacetic acid (7.3 g, 36 mmol) in acetic anhydride (50 mL) was stirred for 1 h at 130° C. After cooling to room temperature, the mixture was diluted with THF (10 mL) before K₂CO₃ (5.0 g, 36 mmol) was added. The resulting mixture was stirred at 60° C. overnight. After cooling to 0° C., the reaction was quenched with MeOH (5 mL) and the mixture was stirred for 30 min at 0° C. Saturated NH₄Cl solution (10 ml, aq.) and DCM (10 mL) were added and stirring continued for 20 min at 0° C. Phase separation followed by extraction of the aqueous layer with DCM (3×200 mL) gave a combined organic phase, which was dried over Na₂SO₄. The crude product was purified by silica gel chromatography (10 g column; eluting with petroleum ether/ethyl acetate; ratio: 1/1) to give tetrahydro-1H-cyclopenta[c]furan-1,5(3H)-dione (3.5 g, 25 mmol) as a pale yellow solid. ¹H NMR (400 MHz, Chloroform-d) δ 4.54 (dd, J=9.6, 5.9 Hz, 1H), 4.25 (dd, J=9.6, 1.9 Hz, 1H), 3.44-3.23 (m, 2H), 2.82-2.54 (m, 3H), 2.35-2.14 (m, 1H).

### Step 4. Synthesis of (±)-(3aS,5R,6aR)-5-((4-methoxybenzyl)amino)hexahydro-1H-cyclopenta[c]furan-1-one

To a mixture of tetrahydro-1H-cyclopenta[c]furan-1,5(3H)-dione (3.5 g, 25 mmol) a nd (4-methoxyphenyl) methanamine (4.1 g, 30 mmol) in MeOH (20 mL) was added NaBH₃ CN (2.4 g, 37 mmol) in portions at 0° C. The resulting mixture was stirred for 1 h at room temperature. The reaction mixture was diluted with water (120 mL), and the aqueous phase w as extracted with ethyl acetate (150 mL) three times. The combined organic layers were washed with brine, dried over sodium sulfate, filtered and concentrated in vacuo. The resulting c rude material was purified by flash chromatography (acetonitrile/water) to give (±)-(3aS,5R,6aR)-5-((4-methoxybenzyl)amino)hexahydro-1H-cyclopenta[c]furan-1-one (850 mg, 3.25 m mol) as colorless oil. LCMS RT 0.451 min, [M+H]⁺ 262, LCMS method C.

### Step 5. Synthesis of (±)-tert-butyl (4-methoxybenzyl)((3aS,5R,6aR)-1-oxohexahydro-1H-cyclopenta[c]furan-5-yl)carbamate

To a mixture of (3aS,5R,6aR)-5-((4-methoxybenzyl)amino) hexahydro-1H-cyclopenta[c]furan-1-one (630 mg, 2.41 mmol) and triethylamine (732 mg, 7.23 mmol) in DCM (10 mL) was added di-tert-butyl dicarbonate (789 mg, 3.62 mmol) dropwise at 0° C. The mixture was stirred for 2 hours at room temperature. The reaction mixture was diluted with water (20 mL), and the aqueous phase was extracted with ethyl acetate (30 mL) three times. The combined organic layers were washed with brine, dried over sodium sulfate, filtered and concentrated in vacuo. The resulting crude material was purified by flash chromatography (acetonitrile/water) to give (±)-tert-butyl (4-methoxybenzyl) ((3aS,5R,6aR)-1-oxohexahydro-1H-cyclopenta[c]furan-5-yl) carbamate (500 mg, 1.38 mmol, 57.4%) as an off-white solid. LCMS RT 1.178 min), [M+H]⁺=361, LCMS method C.

### Step 6. Synthesis of (±)-(1S,2R,4S)—N—((S)-(2,3-dichloro-6-fluorophenyl)(1-methylcyclopentyl)methyl)-2-(hydroxymethyl)-4-((4-methoxybenzyl)amino)cyclopentane-1-carboxamide

To a mixture of (+)-tert-butyl (4-methoxybenzyl)((3aS,5R,6aR)-1-oxohexahydro-1H-cyclopenta[c]furan-5-yl)carbamate (450 mg, 1.25 mmol) in THF (5 mL) was added trimeth ylaluminum (359 mg, 4.98 mmol) dropwise at 0° C. under a nitrogen atmosphere. The mixtu re was stirred for 15 min at 0° C. prior to the addition of (S)-(2,3-dichloro-6-fluorophenyl) (1-methylcyclopentyl) methanamine (1.38 g, 4.98 mmol). The mixture was stirred for 2 h at 50° C. The reaction mixture was diluted with water (10 mL), and the aqueous phase was extrac ted with ethyl acetate (15 mL) three times. The combined organic layers were washed with b rine, dried over sodium sulfate, filtered and concentrated in vacuo. The residue was purified by reverse phase flash chromatography (column: C18 silica gel; mobile phase A: water, mob ile phase B: acetonitrile; gradient: 0% to 100% B in 10 min; detector: UV 220 nm) to give (±)-(1S,2R,4S)—N—((S)-(2,3-dichloro-6-fluorophenyl)(1-methylcyclopentyl)methyl)-2-(hydro xymethyl)-4-((4-methoxybenzyl)amino)cyclopentane-1-carboxamide (150 mg, 279 μmol) as a yellow oil. LCMS RT 0.909 min, [M+H]⁺ 537.20, LCMS method C.

### Step 7. Synthesis of (±)-(1S,2R,4S)-4-amino-N—((S)-(2,3-dichloro-6-fluorophenyl)(1-methylcyclopentyl)methyl)-2-(hydroxymethyl)cyclopentane-1-carboxamide

A mixture of (±)-(1S,2R,4S)—N—((S)-(2,3-dichloro-6-fluorophenyl)(1-methylcyclope ntyl)methyl)-2-(hydroxymethyl)-4-((4-methoxybenzyl)amino)cyclopentane-1-carboxamide (120 mg, 223 μmol) and Ce(NH₄)₂(NO₃)₆ (1.22 g, 2.23 mmol) in acetonitrile (10 mL) was sti rred for 12 h at room temperature. The mixture was concentrated. The resulting crude materi al was purified by C18 flash (acetonitrile/water) to give (±)-(1S,2R,4S)-4-amino-N—((S)-(2,3-dichloro-6-fluorophenyl)(1-methylcyclopentyl)methyl)-2-(hydroxymethyl)cyclopentane-1-carboxamide (50 mg, 0.12 mmol) as a colorless oil. LCMS RT 0.750 min, [M+H]⁺ 417, LC MS method C.

### Step 8. Synthesis of (1S,2R,4S)-4-acetamido-N—((S)-(2,3-dichloro-6-fluorophenyl)(1-methylcyclopentyl)methyl)-2-(hydroxymethyl)cyclopentane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide

A mixture of (±)-(1S,2R,4S)-4-amino-N—((S)-(2,3-dichloro-6-fluorophenyl)(1-methy lcyclopentyl)methyl)-2-(hydroxymethyl)cyclopentane-1-carboxamide (45 mg, 0.11 mmol), TEA (45 μL, 0.32 mmol), acetic acid (13 mg, 0.22 mmol) and T₃P (51 mg, 0.16 mmol) in D MF (2 mL) was stirred for 1 h at room temperature. The reaction mixture was diluted with w ater (10 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Shield RP18 OBD Column, 30*150 mm, 5 μm; mobile phase A: water (10 mM NH₄HCO₃+0.1% NH₄OH), mobile phase B: acetonitrile; flow rate: 60 mL/min; gradient 31% B to 51% B in 8 min, then 51% B; wavelength: 220/254 nm; RT1 (min): 7.40) to give (±)-(1R,2S,4R)-4-acetamido-N—((S)-(2,3-dichloro-6-fluorophenyl)(1-methylcyclopentyl)me thyl)-2-(hydroxymethyl)cyclopentane-1-carboxamide (17 mg, 37 μmol) as a colorless oil. L CMS RT 1.077 min, [M+H]⁺ 459, LCMS method C. The material was further purified by c hiral preparative HPLC (column: CHIRALPAK IC, 2*25 cm, 5 μm; mobile phase A: hexan e (0.5% 2 M NH₃ in MeOH), mobile phase B: EtOH:DCM 1:1; flow rate: 20 mL/min; gra dient: 15% B isocratic; wavelength: 220/254 nm; RT1 (min): 15.95; RT2 (min): 21.01; sam ple solvent: EtOH:DCM 1:1; injection volume: 1 mL) to give (1S,2R,4S)-4-acetamido-N—((S)-(2,3-dichloro-6-fluorophenyl)(1-methylcyclopentyl)methyl)-2-(hydroxymethyl)cyclope ntane-1-carboxamide and (1R,2S,4R)-4-acetamido-N—((S)-(2,3-dichloro-6-fluorophenyl)(1-methylcyclopentyl)methyl)-2-(hydroxymethyl)cyclopentane-1-carboxamide, both as an off-white amorphous solid. Isomer 1 is 1 mg, 2 μmol. ¹HNMR (400 MHz, DMSO-d₆) δ 8.08 (d, J=8.7 Hz, 1H), 7.79 (d, J=7.1 Hz, 1H), 7.61 (dd, J=9.0, 4.9 Hz, 1H), 7.24 (t, J=9.9 Hz, 1H), 5.49 (d, J=8.5 Hz, 1H), 4.22 (t, J=5.1 Hz, 1H), 4.17 (s, 1H), 3.10 (d, J=7.4 Hz, 1H), 3.06-2.99 (m, 1H), 2.77 (q, J=6.7, 4.7 Hz, 1H), 2.29 (q, J=9.5, 8.7 Hz, 1H), 2.07 (dt, J=13.8, 6.7 Hz, 1H), 1.75 (s, 3H), 1.71 (t, J=7.0 Hz, 1H), 1.59 (s, 6H), 1.54-1.44 (m, 2H), 1.36 (s, 1H), 1.25 (s, 1H), 0.95 (s, 3H). LCMS RT 0.911 min, [M+H]⁺459, LCMS method C. Isomer 22 is 2 mg, 4 μmol. ¹HNMR (400 MHz, DMSO-d₆) δ 8.11 (d, J=8.5 Hz, 1H), 7.80 (d, J=7.0 Hz, 1H), 7.61 (dd, J=9.0, 5.0 Hz, 1H), 7.25 (t, J=9.9 Hz, 1H), 5.46 (d, J=8.4H z, 1H), 4.50 (t, J=5.2 Hz, 1H), 4.14 (s, 1H), 3.44-3.36 (m, 1H), 3.21 (td, J=9.6, 5.6 Hz, 1H), 3.03 (q, J=8.0, 7.5 Hz, 1H), 2.42-2.32 (m, 1H), 1.93 (dd, J=13.1, 6.9 Hz, 1H), 1.74 (s, 3H), 1.61 (s, 6H), 1.53 (d, J=8.9 Hz, 2H), 1.40 (t, J=6.8 Hz, 2H), 1.24 (s, 1H), 1.00-0.91 (m, 3H). LCMS RT 0.933 min, [M+H]⁺ 459, LCMS method C.

### Example 20

### (1S,3R,4S)—N—((S)-(3-chloro-2,6-difluorophenyl)(4-fluorobicyclo[2.2.1]heptan-1-yl)meth yl)-3-hydroxy-4-isopropoxycyclopentane-1-carboxamide, (1R,3S,4R)—N—((S)-(3-chloro-2,6-difluorophenyl)(4-fluorobicyclo[2.2.1]heptan-1-yl)methyl)-3-hydroxy-4-isopropoxy cyclopentane-1-carboxamide, (1R,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1S,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide

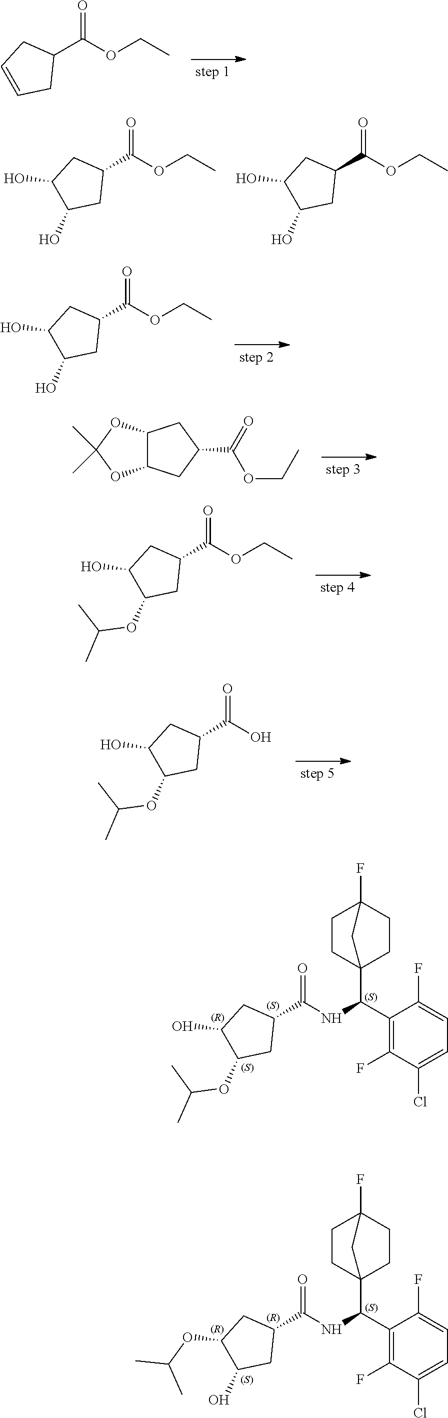

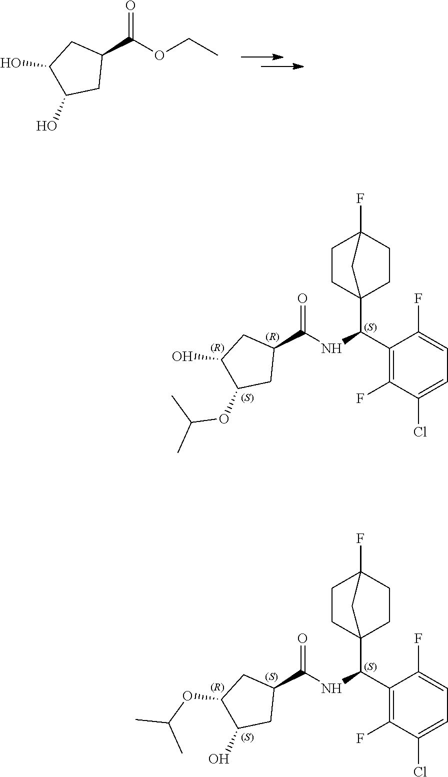

### Step 1. Synthesis of ethyl (1r,3R,4S)-3,4-dihydroxycyclopentane-1-carboxylate and ethyl (1s,3R,4S)-3,4-dihydroxycyclopentane-1-carboxylate

To a stirred mixture of ethyl cyclopent-3-ene-1-carboxylate (5 g, 0.04 mol) and NMO (5 g, 0.04 mol) in acetone (10 mL) and H₂O (10 mL) was added K₂OsO₂(OH)₄ (3 g, 7 mmol) at room temperature under a nitrogen atmosphere. The resulting mixture was stirred overnight at room temperature. The mixture was extracted with DCM (3×250 mL). The combined organic layers were washed with brine (1×100 mL), dried over anhydrous Na₂SO₄. After filtration, the filtrate was concentrated under reduced pressure. The residue was purified by silica gel column chromatography, eluting with petroleum ether:ethyl acetate (1:5) to afford ethyl (3R,4S)-3,4-dihydroxycyclopentane-1-carboxylate (4.14 g, 23.8 mmol, including 1.5 g isomer 1, 420 mg isomer 2, and 2.2 g mixture of the two) as a yellow oil. Isomer 1: ¹H NMR (400 MHz, DMSO-d6) δ 4.47 (d, J=4.2 Hz, 2H), 4.04 (q, J=7.1 Hz, 2H), 3.88 (h, J=4.0 Hz, 2H), 2.95 (tt, J=9.6, 6.7 Hz, 1H), 1.90-1.70 (m, 4H), 1.17 (t, J=7.1 Hz, 3H). Isomer 2: ¹H NMR (400 MHz, DMSO-d6) δ 4.37 (d, J=4.3 Hz, 2H), 4.04 (dd, J=7.1, 3.2 Hz, 2H), 3.76 (dp, J=7.5, 4.5 Hz, 2H), 2.67 (tt, J=9.3, 8.0 Hz, 1H), 1.95 (tdd, J=9.4, 4.8, 1.7 Hz, 2H), 1.83-1.76 (m, 2H), 1.17 (t, J=7.1 Hz, 3H).

### Step 2. Synthesis of ethyl (3aR,5r,6aS)-2,2-dimethyltetrahydro-4H-cyclopenta[d][1,3]dioxole-5-carboxylate

To a stirred mixture of ethyl (1r,3R,4S)-3,4-dihydroxycyclopentane-1-carboxylate (500 mg, 2.87 mmol, isomer 2) and 2,2-dimethoxypropane (299 mg, 2.87 mmol) in acetone (1 mL) was added 4-methylbenzene-1-sulfonic acid (98.9 mg, 574 μmol) at 25° C. under a nitrogen atmosphere. The resulting mixture was stirred for 16 hours at 25° C. under nitrogen. The resulting mixture was extracted with EtOAc (3×20 mL). The combined organic layers were washed with water (1×20 mL) and brine (1×20 mL), and dried over anhydrous Na₂SO₄. After filtration, the filtrate was concentrated under reduced pressure to afford ethyl (3aR,5r,6aS)-2,2-dimethyltetrahydro-4H-cyclopenta[d][1,3]dioxole-5-carboxylate (649 mg, 3.03 mmol, crude) as a colorless oil. ¹H NMR (400 MHz, DMSO-d6) δ 4.62 (dd, J=3.7, 1.6 Hz, 2H), 4.06 (q, J=7.1 Hz, 2H), 2.89-2.80 (m, 1H), 1.98-1.87 (m, 2H), 1.67-1.59 (m, 2H), 1.34 (s, 3H), 1.23-1.12 (m, 6H).

### Step 3. Synthesis of (±)-ethyl (1S,3R,4S)-3-hydroxy-4-isopropoxycyclopentane-1-carboxylate

To a stirred mixture of ethyl (3aR,5r,6aS)-2,2-dimethyltetrahydro-4H-cyclopenta[d][1,3]dioxole-5-carboxylate (200 mg, 0.93 mmol) and triethylsilane (139 mg, 1.20 mmol) in DCM (5 mL) was added TiCl₄ (1.02 mL, 1 M in DCM, 1.02 mmol) dropwise at −40° C. under a nitrogen atmosphere. The resulting mixture was stirred at −40° C. for 1 hour under nitrogen. The reaction was quenched with water/ice at 0° C. The resulting mixture was extracted with DCM (3×50 mL). The combined organic layers were washed with brine (1×100 mL) and NaHCO₃ (1×100 mL), and dried over anhydrous Na₂SO₄. After filtration, the filtrate was concentrated under reduced pressure. The residue was purified by silica gel column chromatography, eluting with petroleum ether:ethyl acetate (5:1) to afford (±)-ethyl (1S,3R,4S)-3-hydroxy-4-isopropoxycyclopentane-1-carboxylate (120 mg, 555 μmol) as a colorless oil. ¹H NMR (400 MHz, DMSO-d6) δ 4.16 (d, J=4.4 Hz, 1H), 4.05 (q, J=7.1 Hz, 2H), 3.98 (q, J=4.3 Hz, 1H), 3.77 (td, J=6.9, 3.7 Hz, 1H), 3.70-3.64 (m, 1H), 3.00-2.87 (m, 1H), 1.94-1.76 (m, 4H), 1.17 (t, J=7.1 Hz, 3H), 1.09 (t, J=6.3 Hz, 6H).

### Step 4. Synthesis of (±)-(1S,3R,4S)-3-hydroxy-4-isopropoxycyclopentane-1-carboxylic acid

To a stirred mixture of (±)-ethyl (1S,3R,4S)-3-hydroxy-4-isopropoxycyclopentane-1-carboxylate (120 mg, 555 μmol) in MeOH (2 mL) and H₂O (2 mL) was added NaOH (44.4 mg, 1.11 mmol) at 25° C. under a nitrogen atmosphere. The resulting mixture was stirred for 1 hour at 25° C. under nitrogen. The mixture was acidified to pH 4 with conc. HCl. The resulting mixture was extracted with DCM (3×250 mL). The combined organic layers were washed with brine (1×100 mL) and dried over anhydrous Na₂SO₄. After filtration, the filtrate was concentrated under reduced pressure to afford (±)-(1S,3R,4S)-3-hydroxy-4-isopropoxycyclopentane-1-carboxylic acid (110 mg, 584 μmol). 1H NMR (300 MHz, DMSO-d6) δ 12.05 (s, 1H), 4.13 (d, J=4.4 Hz, 1H), 3.97 (p, J=4.3 Hz, 1H), 3.76 (td, J=7.0, 3.7 Hz, 1H), 3.70-3.62 (m, 1H), 2.87 (qd, J=8.6, 5.5 Hz, 1H), 1.95-1.74 (m, 4H), 1.09 (dd, J=6.1, 4.7 Hz, 6H).

### Step 5. Synthesis of (1S,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1R,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide

To a stirred mixture of (±)-(1S,3R,4S)-3-hydroxy-4-isopropoxycyclopentane-1-carboxylic acid (100 mg, 531 μmol) and (S)-(3-chloro-2,6-difluorophenyl)(4-fluorobicyclo[2.2.1]heptan-1-yl)methanamine (169 mg, 584 μmol) in DMF (5 mL) was added T3P (507 mg, 50% wt. in EtOAc, 797 μmol) and TEA (69.9 mg, 691 μmol) at 25° C. under a nitrogen atmosphere. The resulting mixture was stirred for 1 hour at 25° C. under nitrogen. The resulting mixture was purified by preparative HPLC with the following conditions (column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 35% B to 65% B in 30 min; wavelength: 254 nm/220 nm; RT (min): 9.58) to afford the desired product (140 mg, 304 μmol) as a white solid, which was further purified by preparative chiral HPLC (column: CHIRAL ART Cellulose-SZ, 3*25 cm, 5 μm; mobile phase A: hexane (0.5% of 2 M NH₃ in MeOH), mobile phase B: EtOH; flow rate: 40 mL/min; gradient: 10% B isocratic; wavelength: 254/220 nm; RT1 (min): 8.63; RT2 (min): 10.525; sample solvent: EtOH:DCM 1:1) to give (1S,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1R,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide, both as a white solid. Isomer 1: 34.6 mg, 73.4 μmol, LCMS RT 1.700 min, [M+H]⁺ 460.20, LCMS method F, ¹H NMR (400 MHz, DMSO-d6) δ 8.20 (d, J=8.4 Hz, 1H), 7.58 (td, J=8.7, 5.4 Hz, 1H), 7.16 (td, J=9.4, 1.6 Hz, 1H), 5.27 (d, J=8.3 Hz, 1H), 4.44 (d, J=5.1 Hz, 1H), 3.89 (p, J=4.7 Hz, 1H), 3.72-3.62 (m, 2H), 2.66 (qd, J=8.9, 6.2 Hz, 1H), 2.05-1.39 (m, 14H), 1.09 (dd, J=12.0, 6.0 Hz, 6H). Isomer 2: 46.0 mg, 97.5 μmol, LCMS RT 1.696 min, [M+H]⁺ 460.15, LCMS method F. ¹H NMR (300 MHz, DMSO-d6) δ 8.20 (d, J=8.3 Hz, 1H), 7.58 (td, J=8.7, 5.5 Hz, 1H), 7.16 (td, J=9.5, 1.7 Hz, 1H), 5.26 (d, J=8.2 Hz, 1H), 4.45 (d, J=5.2 Hz, 1H), 3.90 (p, J=4.6 Hz, 1H), 3.73-3.59 (m, 2H), 2.68 (qd, J=8.9, 6.1 Hz, 1H), 2.06-1.54 (m, 12H), 1.46 (s, 2H), 1.08 (dd, J=13.2, 6.1 Hz, 6H).

Similarly, (1R,3R,4S)—N—((S)-(3-chloro-2,6-difluorophenyl)(4-fluorobicyclo[2.2.1]heptan-1-yl)methyl)-3-hydroxy-4-isopropoxycyclopentane-1-carboxamide and (1S,3S,4R)—N—((S)-(3-chloro-2,6-difluorophenyl)(4-fluorobicyclo[2.2.1]heptan-1-yl)methyl)-3-hydroxy-4-isopropoxycyclopentane-1-carboxamide, both as a white solid, can be prepared from ethyl (1s,3R,4S)-3,4-dihydroxycyclopentane-1-carboxylate after chiral separation by preparative chiral HPLC with the following conditions (column: Lux Cellulose-4, 2.12*25 cm, 5 μm; mobile phase A: hexane (0.5% of 2 M NH₃ in MeOH), mobile phase B: EtOH; flow rate: 20 mL/min; gradient: 3% B isocratic; wavelength: 210/220 nm; RT1 (min): 5.49; RT2 (min): 7.80; sample solvent: EtOH; injection volume: 0.4 mL). Isomer 3: 22.2 mg, 47.4 μmol. LCMS RT 1.534 min, [M+H]⁺ 460.20, LCMS method F, ¹H NMR (400 MHz, DMSO-d6) δ 8.22 (d, J=8.2 Hz, 1H), 7.56 (td, J=8.7, 5.5 Hz, 1H), 7.15 (td, J=9.5, 1.6 Hz, 1H), 5.24 (d, J=8.1 Hz, 1H), 4.07 (d, J=4.3 Hz, 1H), 3.95 (p, J=4.2 Hz, 1H), 3.72-3.58 (m, 2H), 3.06 (ddd, J=15.8, 8.9, 6.2 Hz, 1H), 1.84-1.54 (m, 12H), 1.45 (d, J=9.2 Hz, 2H), 1.06 (dd, J=8.9, 6.1 Hz, 6H). Isomer 4: 34.2 mg, 72.0 μmol, LCMS RT 1.662 min, [M+H]⁺ 460.20, LCMS method F, ¹H NMR (400 MHz, DMSO-d6) δ 8.21 (d, J=8.1 Hz, 1H), 7.56 (td, J=8.7, 5.4 Hz, 1H), 7.19-7.08 (m, 1H), 5.24 (d, J=8.1 Hz, 1H), 4.06 (d, J=4.1 Hz, 1H), 3.92 (p, J=4.1 Hz, 1H), 3.73 (td, J=6.8, 3.6 Hz, 1H), 3.64 (h, J=6.1 Hz, 1H), 3.14-3.00 (m, 1H), 1.87-1.66 (m, 10H), 1.59 (d, J=8.5 Hz, 1H), 1.48 (ddd, J=20.7, 10.3, 6.7 Hz, 3H), 1.08 (t, J=5.7 Hz, 6H).

### Example 21

### Synthesis of N-((1S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide

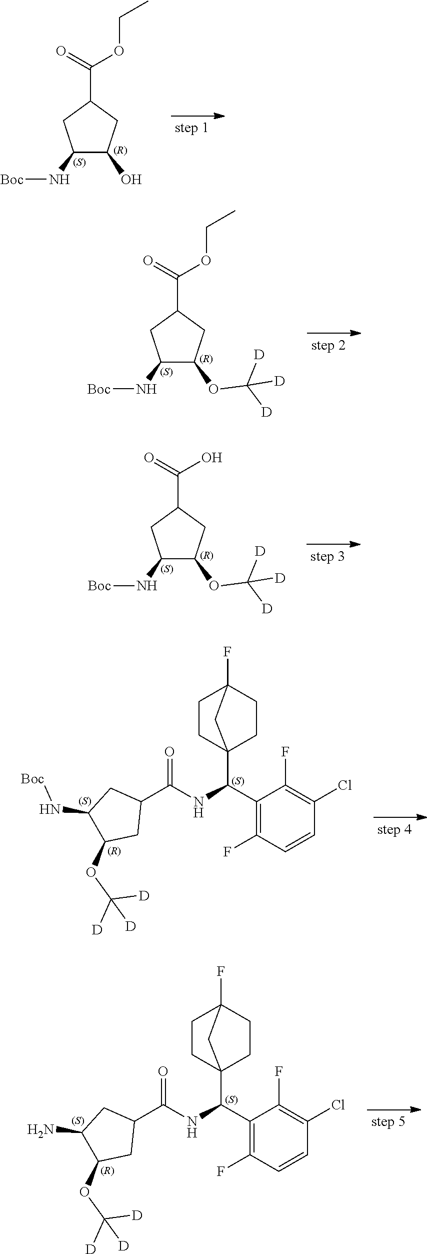

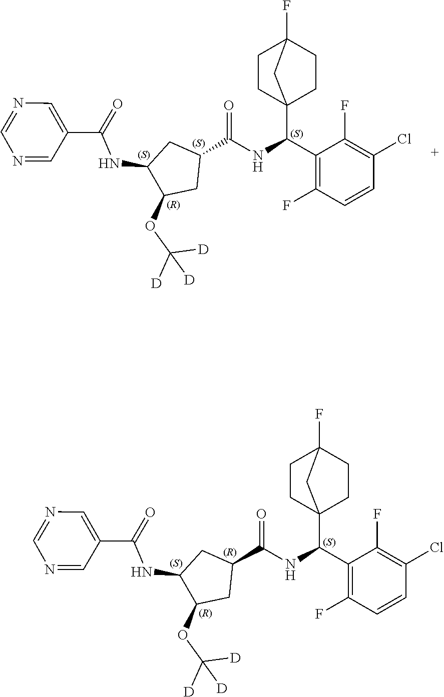

### Step 1. Synthesis of ethyl (3S,4R)-3-((tert-butoxycarbonyl)amino)-4-(methoxy-d3)cyclopentane-1-carboxylate

To a stirred solution of ethyl (3S,4R)-3-((tert-butoxycarbonyl)amino)-4-hydroxycyclopentane-1-carboxylate (600 mg, 2.20 mmol) and Ag₂O (5.09 g, 22.00 mmol) in DCE (30 mL) was added iodomethane-d₃ (1.59 g, 11.00 mmol) dropwise at room temperature under a nitrogen atmosphere. The resulting mixture was stirred for 12 hours at 80° C. under nitrogen. The mixture was cooled to room temperature and filtered. The filter cake was washed with CH₂Cl₂ (3×100 mL). The filtrate was concentrated under reduced pressure. The residue was purified by silica gel column chromatography, eluting with petroleum ether/EtOAc (0% to 50% of EtOAc over 30 min) to afford ethyl (3S,4R)-3-((tert-butoxycarbonyl)amino)-4-(methoxy-d3)cyclopentane-1-carboxylate (400 mg, 1.30 mmol) as a light yellow solid. ¹H NMR (400 MHz, DMSO-d6) δ 6.49 (d, J=8.2 Hz, 1H), 4.05 (q, J=7.1 Hz, 2H), 3.91-3.80 (m, 1H), 3.68-3.61 (m, 1H), 2.90-2.76 (m, 1H), 2.04-1.90 (m, 2H), 1.79-1.72 (m, 2H), 1.38 (s, 9H), 1.17 (t, J=7.1 Hz, 3H).

### Step 2. Synthesis of (3S,4R)-3-((tert-butoxycarbonyl)amino)-4-(methoxy-d3)cyclopentane-1-carboxylic acid

To a stirred solution of ethyl (3S,4R)-3-((tert-butoxycarbonyl)amino)-4-(methoxy-d3)cyclopentane-1-carboxylate (290 mg, 999 μmol) in THF (3 mL) and H₂O (1 mL) was added LiOH (71 mg, 3.00 mmol) at room temperature under a nitrogen atmosphere. The resulting mixture was stirred for 4 hours at 30° C. under nitrogen. The mixture was acidified to pH 5 with HCl (1 N, aq.). The resulting mixture was extracted with EtOAc (3×100 mL). The combined organic layers were washed with brine (3×50 mL), and dried over anhydrous Na₂SO₄. After filtration, the filtrate was concentrated under reduced pressure. The crude product (3S,4R)-3-((tert-butoxycarbonyl)amino)-4-(methoxy-d3)cyclopentane-1-carboxylic acid (200 mg, 762 μmol) was used in the next step directly without purification. LCMS RT 0.539 min, [M+H]⁺=263.1, LCMS method G.

### Step 3. Synthesis of tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate

To a stirred solution of (3S,4R)-3-((tert-butoxycarbonyl)amino)-4-(methoxy-d3)cyclopentane-1-carboxylic acid (80 mg, 0.30 mmol) and (S)-(3-chloro-2,6-difluorophenyl)(4-fluorobicyclo[2.2.1]heptan-1-yl)methanamine (88 mg, 0.30 mmol) in DMF (2 mL) was added sodium bicarbonate (77 mg, 0.91 mmol) and HATU (170 mg, 0.46 mmol) at room temperature under a nitrogen atmosphere. The resulting mixture was stirred for 3 hours at 30° C. under nitrogen. After concentration in vacuo, the residue was purified by reversed-phase flash chromatography (column: C18 gel; mobile phase A: water (0.1% NH₄OH), mobile phase B: acetonitrile; gradient: 10% to 90% B in 40 min; detector: UV 254/220 nm) to give tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate (80 mg, 0.14 mmol) as a white solid. LCMS RT 1.291 min, m/z [M−H]⁻ 532.2, LCMS method G.

### Step 4. Synthesis of (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride

To a stirred solution of tert-butyl ((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)carbamate (40 mg, 75.00 μmol) in 1,4-dioxane (1 mL) was added HCl in 1,4-dioxane (4 M, 1 mL) dropwise at room temperature under a nitrogen atmosphere. The resulting mixture was stirred for 2 hours at 30° C. under nitrogen. The mixture was concentrated and triturated with Et₂O (2 mL) twice. The crude product (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride (40 mg, crude) was used in the next step directly without further purification. LCMS RT 0.985 min, [M+H]⁺ 434.2, LCMS method G.

### Step 5. Synthesis of N-((1S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide

To a stirred solution of (3S,4R)-3-amino-N—((S)-(3-chloro-2,6-difluorophenyl)(4-fluorobicyclo[2.2.1]heptan-1-yl)methyl)-4-(methoxy-d3)cyclopentane-1-carboxamide hydrochloride (30 mg, 69 μmol) and pyrimidine-5-carboxylic acid (9 mg, 69 μmol) in DMF (1 mL) was added sodium bicarbonate (17 mg, 0.21 mmol) and HATU (39 mg, 0.10 mmol) at room temperature under a nitrogen atmosphere. The resulting mixture was stirred for 3 hours at 30° C. under nitrogen. After concentration under reduced pressure, the residue was purified by reversed-phase flash chromatography (column: C18 gel; mobile phase A: water (0.1% NH₄OH), mobile phase B: acetonitrile; gradient: 10% to 90% B in 40 min; detector: UV 254/220 nm) to give N-((1S,2R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide (20 mg, 51%) as a white solid. LCMS RT 1.177 min, [M+H]⁺ 540.2, LCMS method. It was further purified by preparative chiral HPLC with the following conditions (column: CHIRALPAK IA, 2*25 cm, 5 μm; mobile phase A: hexane (0.5% of 2 M NH₃ in MeOH), mobile phase B: EtOH; flow rate: 20 mL/min; gradient: 50% B isocratic; wavelength: 200/215 nm; RT1 (min): 6.3; RT2 (min): 17.48; sample solvent: EtOH:CH₂Cl₂ 1:1; injection volume: 1 mL) to give N-((1S,2R,4S)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide and N-((1S,2R,4R)-4-(((S)-(3-chloro-2,6-difluorophenyl)(4-fluorobicyclo[2.2.1]heptan-1-yl)methyl)carbamoyl)-2-(methoxy-d3)cyclopentyl)pyrimidine-5-carboxamide, both as a white solid. Isomer 1: 7.1 mg, 13 μmol, LCMS RT 1.875 min, [M+H]⁺ 540.25, LCMS method F, ¹H NMR (400 MHz, DMSO-d6) δ 9.30 (d, J=3.2 Hz, 1H), 9.17-9.08 (m, 2H), 8.57 (d, J=7.7 Hz, 1H), 8.28 (d, J=8.1 Hz, 1H), 7.58 (td, J=8.7, 5.4 Hz, 1H), 7.16 (t, J=9.5 Hz, 1H), 5.30 (d, J=8.2 Hz, 1H), 4.30 (dt, J=12.6, 6.2 Hz, 1H), 3.77 (s, 1H), 3.16-3.02 (m, 1H), 2.07-1.93 (m, 3H), 1.86-1.56 (m, 9H), 1.46 (s, 2H). ¹⁹F NMR (282 MHz, DMSO) δ −111.047, −113.597, −173.563. Isomer 2: 3.0 mg, 5.5 μmol, LCMS RT 1.875 min, [M+H]⁺ 540.25, LCMS method F. ¹H NMR (400 MHz, DMSO-d6) δ 9.30 (s, 1H), 9.14 (d, J=1.4 Hz, 2H), 8.56 (d, J=8.0 Hz, 1H), 8.28 (d, J=8.3 Hz, 1H), 7.57 (td, J=8.7, 5.5 Hz, 1H), 7.23-7.09 (m, 1H), 5.29 (d, J=8.2 Hz, 1H), 4.35-4.18 (m, 1H), 3.80 (td, J=4.5, 2.4 Hz, 1H), 3.10 (d, J=10.5 Hz, 1H), 2.12 (ddd, J=13.6, 8.8, 2.5 Hz, 1H), 1.94 (q, J=10.5 Hz, 1H), 1.85-1.68 (m, 9H), 1.61 (d, J=8.4 Hz, 1H), 1.47 (d, J=8.0 Hz, 2H). ¹⁹F NMR (282 MHz, DMSO) δ −111.635, −113.401, −173.565.

### Example 22

### (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide

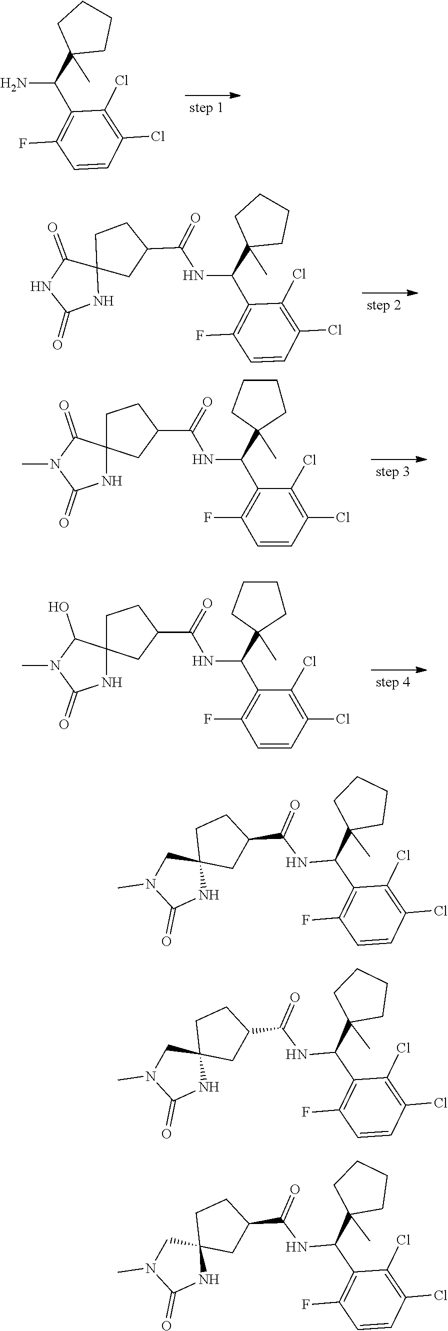

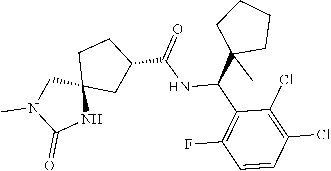 [SMILES: CN1C[C@@]2(CC[C@H](C(=O)N[C@H](c3c(F)ccc(Cl)c3Cl)C3(C)CCCC3)C2)NC1=O]

### Step 1. Synthesis of N—((S)-(2,3-dichloro-6-fluorophenyl)(1-methylcyclopentyl)methyl)-2,4-dioxo-1,3-diazaspiro[4.4]nonane-7-carboxamide

To a mixture of 2,4-dioxo-1,3-diazaspiro[4.4]nonane-7-carboxylic acid (300 mg, 1.51 mmol), (S)-(2,3-dichloro-6-fluorophenyl)(1-methylcyclopentyl)methanamine (418 mg, 1.51 mmol) and TEA (917 mg, 9.08 mmol) in DMF (3 mL) was added T₃P (1.93 g, 50% wt, 3.03 mmol). The mixture was stirred for 2 hours at 25° C. The reaction mixture was diluted with water (20 mL), and the aqueous phase was extracted with ethyl acetate (20 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm). Concentration in vacuo resulted in N—((S)-(2,3-dichloro-6-fluorophenyl)(1-methylcyclopentyl)methyl)-2,4-dioxo-1,3-diazaspiro[4.4]nonane-7-carboxamide (0.23 g, 33%) as a colorless oil. LCMS RT 0.981 min, [M+H]⁺ 456, LC method C.

### Step 2. Synthesis of N—((S)-(2,3-dichloro-6-fluorophenyl)(1-methylcyclopentyl)methyl)-3-methyl-2,4-dioxo-1,3-diazaspiro[4.4]nonane-7-carboxamide

A mixture of N—((S)-(2,3-dichloro-6-fluorophenyl)(1-methylcyclopentyl)methyl)-2,4-dioxo-1,3-diazaspiro[4.4]nonane-7-carboxamide (220 mg, 482 μmol) and 1,1-dimethoxy-N, N-dimethylethan-1-amine (193 mg, 1.45 mmol) in toluene (2 mL) was stirred for 2 hours at 110° C. The reaction mixture was concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm) to give N—((S)-(2,3-dichloro-6-fluorophenyl)(1-methylcyclopentyl)methyl)-3-methyl-2,4-dioxo-1,3-diazaspiro[4.4]nonane-7-carboxamide (0.19 g, 0.40 mmol) as a colorless oil. LCMS RT 0.994 min, [M+H]⁺ 470, LCMS method C.

### Step 3. Synthesis of N—((S)-(2,3-dichloro-6-fluorophenyl)(1-methylcyclopentyl)methyl)-4-hydroxy-3-methyl-2-oxo-1,3-diazaspiro[4.4]nonane-7-carboxamide

To a mixture of N—((S)-(2,3-dichloro-6-fluorophenyl)(1-methylcyclopentyl)methyl)-3-methyl-2,4-dioxo-1,3-diazaspiro[4.4]nonane-7-carboxamide (180 mg, 383 μmol) in THF (3 mL) was added LiAlH₄ (22 mg, 574 μmol) in portions at 0° C. under a nitrogen atmosphere. The mixture was stirred for 2 hours at 25° C. The reaction was then cooled to 0° C. and quenched with water (0.18 mL), sodium hydroxide (0.36 mL, 4 M) and then water (0.18 mL). The mixture was filtered through a pad of Celite. The pad was washed with ethyl acetate, and the filtrate was concentrated in vacuo. The residue was purified by reverse phase flash chromatography (column: C18 silica gel; mobile phase A: water, mobile phase B: acetonitrile; gradient: 0% to 100% in 25 min; detector: UV 254 nm) to give N—((S)-(2,3-dichloro-6-fluorophenyl)(1-methylcyclopentyl)methyl)-3-methyl-2,4-dioxo-1,3-diazaspiro[4.4]nonane-7-carboxamide (0.15 g, 0.32 mmol) as a colorless oil. LCMS RT 0.941 min, [M+H]⁺ 472, LCMS method C.

### Step 4. Synthesis of (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide

To a mixture of N—((S)-(2,3-dichloro-6-fluorophenyl)(1-methylcyclopentyl)methyl)-3-methyl-2,4-dioxo-1,3-diazaspiro[4.4]nonane-7-carboxamide (140 mg, 335 μmol) in THF (2 mL) was added Et₃SiH (78.3 mg, 669 μmol) dropwise at room temperature, and then TFA (76.3 mg, 669 μmol) was added. The mixture was stirred for 2 hours at 70° C. The reaction mixture was concentrated in vacuo. The resulting crude material was purified by preparative HPLC (mobile phase A: water with 0.1% formic acid, mobile phase B: acetonitrile) to give N—((S)-(2,3-dichloro-6-fluorophenyl)(1-methylcyclopentyl)methyl)-3-methyl-2-oxo-1,3-diazaspiro[4.4]nonane-7-carboxamide (0.10 g) as a colorless oil. LCMS RT 1.020 min, [M+H]⁺ 456, LCMS method C.

The product was further purified by chiral preparative HPLC (column: CHIRALPAK IG, 2*25 cm, 5 μm; mobile phase A: hexane, mobile phase B: EtOH; flow rate: 20 mL/min; gradient: 15% B isocratic; wavelength: 220/254 nm; RT1 (min): 9.259; RT2 (min): 11.358; sample solvent: EtOH:DCM 1:1; injection volume: 1.5 mL) to (5R,7R)—N—((R)-(2,3-dichloro-6-fluorophenyl)(1-methylcyclopentyl)methyl)-3-methyl-2-oxo-1,3-diazaspiro[4.4]nonane-7-carboxamide, (5R,7S)—N—((R)-(2,3-dichloro-6-fluorophenyl)(1-methylcyclopentyl)methyl)-3-methyl-2-oxo-1,3-diazaspiro[4.4]nonane-7-carboxamide, (5S,7S)—N—((R)-(2,3-dichloro-6-fluorophenyl)(1-methylcyclopentyl)methyl)-3-methyl-2-oxo-1,3-diazaspiro[4.4]nonane-7-carboxamide and (5S,7R)—N—((R)-(2,3-dichloro-6-fluorophenyl)(1-methylcyclopentyl)methyl)-3-methyl-2-oxo-1,3-diazaspiro[4.4]nonane-7-carboxamide, all as an off-white amorphous solid.

Isomer 1: 6.8 mg, 15 μmol. ¹H NMR (400 MHz, DMSO-d6) δ 8.12 (d, J=8.6 Hz, 1H), 7.61 (dd, J=9.0, 5.1 Hz, 1H), 7.26 (dd, J=10.8, 9.0 Hz, 1H), 6.63 (s, 1H), 5.48 (d, J=8.5 Hz, 1H), 3.17-3.06 (m, 2H), 3.06-2.98 (m, 1H), 2.56 (s, 3H), 1.92 (td, J=9.0, 8.5, 5.6 Hz, 1H), 1.82-1.54 (m, 4H), 1.60 (s, 7H), 1.37 (s, 1H), 1.26 (t, J=9.2 Hz, 1H), 0.97 (d, J=2.8 Hz, 3H), LCMS RT 1.205 min, [M+H]⁺ 456.10, LC method B

Isomer 2: 7.4 mg, 16 μmol. ¹H NMR (400 MHz, DMSO-d6) δ 8.11 (d, J=8.6 Hz, 1H), 7.61 (dd, J=8.9, 5.1 Hz, 1H), 7.26 (dd, J=10.7, 9.0 Hz, 1H), 6.65 (s, 1H), 5.48 (d, J=8.5 Hz, 1H), 3.22 (d, J=8.8 Hz, 1H), 3.12 (d, J=8.8 Hz, 1H), 3.02 (p, J=7.7 Hz, 1H), 2.58 (s, 3H), 1.89 (dd, J=13.2, 9.3 Hz, 1H), 1.78 (dt, J=13.0, 5.9 Hz, 2H), 1.72-1.44 (m, 9H), 1.38 (d, J=6.7 Hz, 1H), 1.31-1.22 (m, 1H), 0.97 (d, J=2.8 Hz, 3H), LCMS RT 1.195 min, [M+H]⁺ 456.10, LCMS method B

Isomer 3: 27.4 mg, 60.0 μmol. ¹H NMR (400 MHz, DMSO-d6) δ 8.08 (d, J=8.7 Hz, 1H), 7.61 (dd, J=8.9, 5.0 Hz, 1H), 7.31-7.21 (m, 1H), 6.45 (s, 1H), 5.50 (d, J=8.5 Hz, 1H), 3.14 (q, J=8.5 Hz, 2H), 2.94 (p, J=8.2 Hz, 1H), 2.51 (p, J=1.8 Hz, 3H), 1.85-1.71 (m, 3H), 1.71-1.50 (m, 9H), 1.41-1.33 (m, 1H), 1.27 (d, J=8.1 Hz, 1H), 0.97 (d, J=2.8 Hz, 3H), LCMS RT 1.210 min, [M+H]⁺, 456.10, LCMS method B

Isomer 4: 27.4 mg, 60.0 μmol. ¹H NMR (400 MHz, DMSO-d6) δ 8.09 (d, J=8.7 Hz, 1H), 7.62 (dd, J=8.9, 5.1 Hz, 1H), 7.26 (dd, J=10.7, 8.9 Hz, 1H), 6.49 (s, 1H), 5.52 (d, J=8.6 Hz, 1H), 3.19 (d, J=8.6 Hz, 1H), 3.13 (d, J=8.5 Hz, 1H), 2.98-2.90 (m, 1H), 2.61 (s, 3H), 1.89 (dd, J=12.4, 7.8 Hz, 1H), 1.79-1.49 (m, 10H), 1.40-1.33 (m, 1H), 1.27 (d, J=8.1 Hz, 1H), 0.96 (d, J=2.8 Hz, 3H), LCMS RT 1.198 min, [M+H]⁺ 456.10, LCMS method B.

### Example 23

### (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide

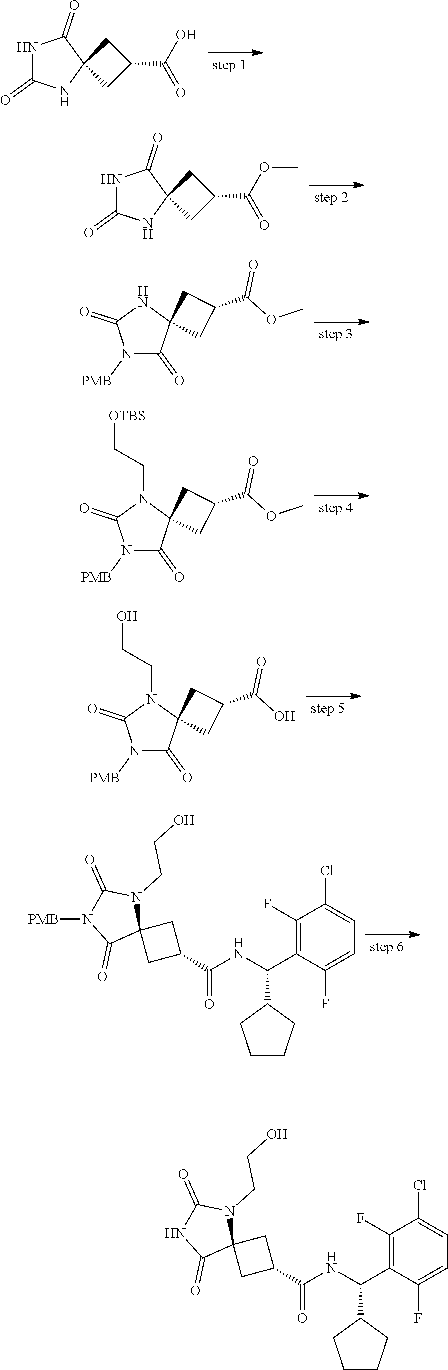

### Step 1. Synthesis of methyl (2r,4r)-6,8-dioxo-5,7-diazaspiro[3.4]octane-2-carboxylate

To a mixture of (2r,4r)-6,8-dioxo-5,7-diazaspiro[3.4]octane-2-carboxylic acid (1 g, 5 mmol) in DCM/MeOH (2:1, 10 mL) was added TMSCHN₂ (8 mL, 2 M, 16 mmol) dropwise at 0° C. under a nitrogen atmosphere. The mixture was stirred for 2 h at room temperature. The reaction was quenched with saturated NH₄Cl (aq.) and the aqueous phase was extracted with DCM (20 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 5% to 40% B in 10 min; detector: UV 220 nm) to afford methyl (2r,4r)-6,8-dioxo-5,7-diazaspiro[3.4]octane-2-carboxylate (500 mg, 2.52 mmol) as a colorless oil. LCMS RT 0.535 min, [M+H]⁺ 199, LCMS method C.

### Step 2. Synthesis of methyl (2s,4s)-7-(4-methoxybenzyl)-6,8-dioxo-5,7-diazaspiro[3.4]octane-2-carboxylate

To a mixture of methyl (2r,4r)-6,8-dioxo-5,7-diazaspiro[3.4]octane-2-carboxylate (1.7 g, 8.6 mmol), Cs₂CO₃ (5.6 g, 17 mmol) in DMF (20 mL) was added 1-(chloromethyl)-4-m ethoxybenzene (1.5 g, 9.4 mmol) dropwise at 0° C. under a nitrogen atmosphere. The mixtur e was stirred for 16 hours at 0° C. The reaction mixture was diluted with water (100 mL), an d the aqueous phase was extracted with ethyl acetate (100 mL) three times. The combined or ganic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 25 min; detector: UV 254 nm) to give methyl (2s,4s)-7-(4-methoxybenzyl)-6,8-dioxo-5,7-diazaspiro[3.4]octane-2-carboxylate (1 g, 3 mmol) as a colorless oil. LCMS RT 0.696 min, [M+H]⁺ 31 9, LCMS method A.

### Step 3. Synthesis of methyl (2s,4s)-5-(2-((tert-butyldimethylsilyl)oxy)ethyl)-7-(4-methoxybenzyl)-6,8-dioxo-5,7-diazaspiro[3.4]octane-2-carboxylate

To a mixture of methyl (2s,4s)-7-(4-methoxybenzyl)-6,8-dioxo-5,7-diazaspiro[3.4]octane-2-carboxylate (290 mg, 911 μmol) and Cs₂CO₃ (594 mg, 1.82 mmol) in DMF (5 mL) was added (2-bromoethoxy)(tert-butyl)dimethylsilane (262 mg, 1.09 mmol) at −78° C. The mixture was stirred for 2 h at room temperature. The reaction mixture was diluted with water (10 ml) and extracted with ethyl acetate (50 mL*3). The combined organic layers were washed with brine, dried over sodium sulfate, filtered and concentrated in vacuo. The resulting crude material was purified by C18 flash (acetonitrile/water) to give methyl (2s,4s)-5-(2-((tert-butyldimethylsilyl)oxy)ethyl)-7-(4-methoxybenzyl)-6,8-dioxo-5,7-diazaspiro[3.4]octane-2-carboxylate (275 mg, 577 μmol) as a colorless oil. LCMS RT 1.456 min, [M+H]⁺ 477, LCMS method C.

### Step 4. Synthesis of (2s,4s)-5-(2-hydroxyethyl)-7-(4-methoxybenzyl)-6,8-dioxo-5,7-diazaspiro[3.4]octane-2-carboxylic acid

A mixture of methyl (2s,4s)-5-{2-[(tert-butyldimethylsilyl)oxy]ethyl}-7-[(4-methoxyphenyl)methyl]-6,8-dioxo-5,7-diazaspiro[3.4]octane-2-carboxylate (1 g, 2.098 mmol) and NaOH (0.25 g, 6.294 mmol) in MeOH (10 mL) was stirred for 1 h at 25° C. The mixture was acidified to pH 5 with HCl (1 N). The precipitated solids were collected by filtration and washed with MeOH to give (2s,4s)-5-(2-hydroxyethyl)-7-[(4-methoxyphenyl)methyl]-6,8-dioxo-5,7-diazaspiro[3.4]octane-2-carboxylic acid (530 mg,) as an off-white solid. LCMS RT 0.640 min, [M+H]⁺ 349, LCMS method C.

### Step 5. Synthesis of (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide

A mixture of (2s,4s)-5-(2-hydroxyethyl)-7-[(4-methoxyphenyl)methyl]-6,8-dioxo-5,7-diazaspiro[3.4]octane-2-carboxylic acid (530 mg, 1.521 mmol), (1S)-1-(3-chloro-2,6-difluorophenyl)-1-cyclopentylmethanamine (373.82 mg, 1.521 mmol), T₃P (726.14 mg, 2.281 mmol) and TEA (461.88 mg, 4.563 mmol) in DCM (8 mL) was stirred for 1 h at 25° C. The reaction mixture was diluted with water (10 mL), and the aqueous phase was extracted with DCM (10 mL) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% B in 10 min; detector: UV 220 nm) to give (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide (540 mg) as an off-white solid. LCMS RT 1.227 min, [M+H]⁺ 576, LCMS method C.

### Step 6. Synthesis of (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide

A mixture of (2r,4S)—N—((S)-(3-chloro-2,6-difluorophenyl)(cyclopentyl)methyl)-5-(2-hydroxyethyl)-7-(4-methoxybenzyl)-6,8-dioxo-5,7-diazaspiro[3.4]octane-2-carboxamide (540 mg, 0.937 mmol) and Ce(NH₄)₂(NO₃)₆ (515.81 mg, 0.937 mmol) in acetonitrile/H₂O (10 mL, 4:1) was stirred for 1 h at 70° C. The reaction mixture was diluted with water (20 mL), and the aqueous phase was extracted with ethyl acetate (20 mL) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% B in 10 min; detector: UV 220 nm) to give (2r,4S)—N—((S)-(3-chloro-2,6-difluorophenyl)(cyclopentyl)methyl)-5-(2-hydroxyethyl)-6,8-dioxo-5,7-diazaspiro[3.4]octane-2-carboxamide (10 mg) as an off-white solid. ¹H NMR (400 MHz, DMSO-d6) δ 10.62 (s, 1H), 8.38 (d, J=7.4 Hz, 1H), 7.53 (td, J=8.6, 5.4 Hz, 1H), 7.21-7.03 (m, 1H), 4.97-4.72 (m, 2H), 3.61 (q, J=5.9 Hz, 2H), 3.42 (t, J=6.1 Hz, 2H), 3.19 (q, J=9.3 Hz, 1H), 2.62 (ddd, J=21.6, 12.6, 8.7 Hz, 3H), 2.47-2.33 (m, 2H), 1.88 (dt, J=12.4, 5.1 Hz, 1H), 1.69-1.42 (m, 4H), 1.32 (ddd, J=26.8, 12.2, 6.2 Hz, 2H), 1.02 (d, J=9.8 Hz, 1H). LCMS RT 1.078 min, [M+H]⁺ 456.10, LCMS method B.

### Example 24

### (1R,3R)-3-acetamido-N—((S)-(2,3-dichloro-6-fluorophenyl)(1-(2-hydroxyethyl)cyclopentyl)methyl)cyclopentane-1-carboxamide

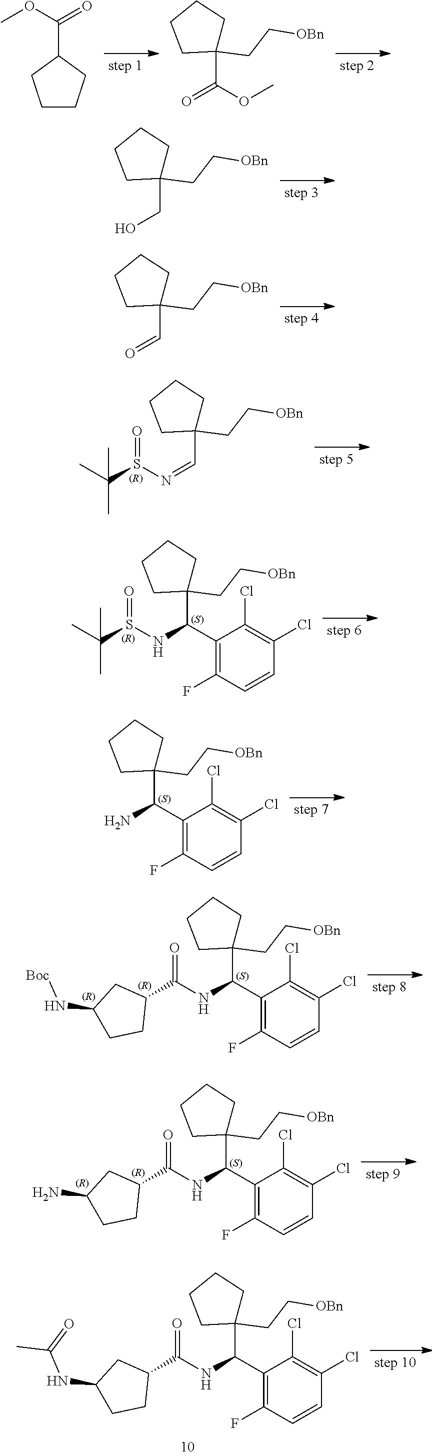

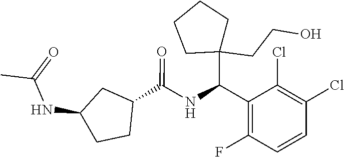 [SMILES: CC(=O)N[C@@H]1CC[C@@H](C(=O)N[C@H](c2c(F)ccc(Cl)c2Cl)C2(CCO)CCCC2)C1]

### Step 1. Synthesis of methyl 1-(2-(benzyloxy)ethyl)cyclopentane-1-carboxylate

To a mixture of methyl cyclopentanecarboxylate (5 g, 0.04 mol) in THF (70 mL) was added LDA (30 mL, 2 molar, 0.06 mol) dropwise at −78° C. under a nitrogen atmosphere. The mixture was stirred for 1 h at −78° C. prior to the addition of ((2-bromoethoxy)methyl) benzene (10 g, 0.05 mol) dropwise at −78° C. The mixture was stirred for 16 h at room temperature. The reaction was quenched with saturated NH₄Cl (aq.) and the aqueous phase was extracted with ethyl acetate (250 mL) three times. The combined organic layers were washed with brine, dried over sodium sulfate, filtered and concentrated in vacuo. The resulting crude material was purified by C18 flash chromatography (mobile phase A: water, mobile phase B: acetonitrile) to give methyl 1-(2-(benzyloxy)ethyl) cyclopentane-1-carboxylate (7.2 g, 27 mmol) as a colorless oil. LCMS RT 1.123 min, [M+H]⁺ 263, LCMS method C.

### Step 2. Synthesis of (1-(2-(benzyloxy)ethyl)cyclopentyl)methanol

To a mixture of methyl 1-(2-(benzyloxy)ethyl) cyclopentane-1-carboxylate (7.1 g, 27 mmol) in THF (100 mL) was added LiAlH₄ (1.2 g, 32 mmol) in portions at 0° C. The mixture was stirred for 2 h at room temperature. The reaction was cooled to 0° C. and quenched with water (1.5 mL), sodium hydroxide (3 mL, 4 N) and water (1.5 mL). The mixture was filtered through a pad of Celite. The pad was washed with DCM, and the filtrate was concentrated in vacuo resulted in (1-(2-(benzyloxy)ethyl)cyclopentyl)methanol (5 g, 0.02 mol) as a colorless oil. LCMS RT 0.988 min, [M+H]⁺ 235, LCMS method C.

### Step 3. Synthesis of 1-(2-(benzyloxy)ethyl)cyclopentane-1-carbaldehyde

To a mixture of (1-(2-(benzyloxy)ethyl)cyclopentyl)methanol (4.9 g, 21 mmol) and molecule sieve 4 Å activated powder (500 mg) in DCM (100 mL) was added PCC (5.4 g, 25 mmol) at 0° C. The mixture was stirred at 0° C. for 2 h. The mixture was diluted with ether/pentane (1:1, 500 mL). The mixture was then filtered through Celite (50 g). The pad was washed with ether. The combined filtrate was concentrated (water bath temperature <15° C.) to ˜2 mL to give 1-(2-(benzyloxy)ethyl)cyclopentane-1-carbaldehyde (5 g, 0.02 mol, crude). LCMS RT 1.107 min, [M+Na]⁺ 255, LCMS method C.

### Step 4. Synthesis of (R)—N-((1-(2-(benzyloxy)ethyl)cyclopentyl)methylene)-2-methylpropane-2-sulfinamide

A mixture of 1-(2-(benzyloxy)ethyl) cyclopentane-1-carbaldehyde (5.5 g, 24 mmol), (R)-2-methylpropane-2-sulfinamide (3.2 g, 26 mmol) and Ti(OⁱPr)₄ (6.7 g, 24 mmol) in THF (100 mL) was stirred for 2 h at 50° C. The reaction mixture was diluted with water (300 mL) and filtrated. The filtrate was extracted with ethyl acetate (350 mL) three times. The combined organic layers were washed with brine, dried over sodium sulfate, filtered and concentrated in vacuo. The resulting crude material was purified by C18 flash chromatography (acetonitrile/water) to give (R)—N-((1-(2-(benzyloxy)ethyl)cyclopentyl)methylene)-2-methylpropane-2-sulfinamide (2.9 g, 8.6 mmol) as a colorless oil. LCMS RT1.210 min, [M+H]⁺ 336, LCMS method C.

### Step 5. Synthesis of (R)—N—((S)-(1-(2-(benzyloxy)ethyl)cyclopentyl)(2,3-dichloro-6-fluorophenyl)methyl)-2-methylpropane-2-sulflnamide

To a mixture of 1,2-dichloro-4-fluorobenzene (1.7 g, 10 mmol) in THF (50 mL) was added LDA (6.3 mL, 2 molar, 13 mmol) dropwise at −78° C. under a nitrogen atmosphere. The mixture was stirred for 1 h at −78° C. prior to the addition of (R)—N-((1-(2-(benzyloxy)ethyl)cyclopentyl)methylene)-2-methylpropane-2-sulfinamide (2.8 g, 8.3 mmol) at −78° C. The mixture was stirred for 16 h at room temperature. The reaction was quenched with saturated NH₄Cl (aq.) and the aqueous phase was extracted with ethyl acetate (300 mL) three times. The combined organic layers were washed with brine, dried over sodium sulfate, filtered and concentrated in vacuo. The resulting crude material was purified by C18 flash chromatography (acetonitrile/water) to give (R)—N—((S)-(1-(2-(benzyloxy)ethyl)cyclopentyl)(2,3-dichloro-6-fluorophenyl)methyl)-2-methylpropane-2-sulfinamide (2.5 g, 5.0 mmol) as a yellow oil. LCMS RT1.342 min, [M+H]⁺ 500, LCMS method C.

### Step 6. Synthesis of (S)-(1-(2-(benzyloxy)ethyl)cyclopentyl)(2,3-dichloro-6-fluorophenyl)methanamine

A mixture of (R)—N—((S)-(1-(2-(benzyloxy)ethyl)cyclopentyl)(2,3-dichloro-6-fluorophenyl)methyl)-2-methylpropane-2-sulfinamide (2.5 g, 5.0 mmol) in HCl (30 ml, 4 N in dioxane) was stirred for 1 h at room temperature. The mixture was concentrated in vacuo to afford (S)-(1-(2-(benzyloxy)ethyl)cyclopentyl)(2,3-dichloro-6-fluorophenyl)methanamine (1.9 g, 4.8 mmol) as a yellow oil. LCMS RT 0.896 min, [M+H]⁺ 396, LCMS method C.

### Step 7. Synthesis of tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate

To a mixture of (S)-(1-(2-(benzyloxy)ethyl)cyclopentyl)(2,3-dichloro-6-fluorophenyl)methanamine (400 mg, 1.01 mmol), (1R,3R)-3-((tert-butoxycarbonyl)amino)cyclopentane-1-carboxylic acid (231 mg, 1.01 mmol), TEA (306 mg, 3.03 mmol) in DMF (8 mL) was added T₃P (642 mg, 2.02 mmol). The mixture was stirred for 1 h at room temperature. The reaction mixture was diluted with water (50 mL), and the aqueous phase was extracted with ethyl acetate (60 mL) three times. The combined organic layers were washed with brine, dried over sodium sulfate, filtered and concentrated in vacuo. The resulting crude material was purified by C18 flash chromatography (acetonitrile/water) to give tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate (360 mg, 593 μmol) as a yellow oil. LCMS RT1.469 min, [M+H]⁺ 607, LCMS method C.

### Step 8. Synthesis of (1R,3R)-3-amino-N—((S)-(1-(2-(benzyloxy)ethyl)cyclopentyl)(2,3-dichloro-6-fluorophenyl)methyl)cyclopentane-1-carboxamide

A mixture of tert-butyl ((1R,3R)-3-(((S)-(1-(2-(benzyloxy)ethyl)cyclopentyl)(2,3-dichloro-6-fluorophenyl)methyl)carbamoyl)cyclopentyl)carbamate (340 mg, 560 μmol) in HCl (5 ml, 4 N in dioxane) was stirred for 1 h at room temperature. The mixture was concentrated in vacuo to afford (1R,3R)-3-amino-N—((S)-(1-(2-(benzyloxy)ethyl)cyclopentyl)(2,3-dichloro-6-fluorophenyl)methyl)cyclopentane-1-carboxamide (230 mg, 453 μmol) as a yellow oil. LCMS RT 0.921 min, [M+H]⁺ 507, LCMS method C.

### Step 9. Synthesis of (1R,3R)-3-acetamido-N—((S)-(1-(2-(benzyloxy)ethyl)cyclopentyl)(2,3-dichloro-6-fluorophenyl)methyl)cyclopentane-1-carboxamide

To a mixture of (1R,3R)-3-amino-N—((S)-(1-(2-(benzyloxy)ethyl)cyclopentyl)(2,3-dichloro-6-fluorophenyl)methyl)cyclopentane-1-carboxamide (200 mg, 394 μmol) and TEA (119 mg, 1.18 mmol) in DCM (5 mL) was added acetyl chloride (30.9 mg, 394 μmol) dropwise at 0° C. The solution was stirred for 1 h at room temperature. The reaction was quenched with water. The aqueous phase was extracted with ethyl acetate (50 mL) three times. The combined organic layers were washed with brine, dried over sodium sulfate, filtered and concentrated in vacuo. The resulting crude material was purified by C18 flash chromatography (acetonitrile/water) to give (1R,3R)-3-acetamido-N—((S)-(1-(2-(benzyloxy)ethyl)cyclopentyl)(2,3-dichloro-6-fluorophenyl)methyl)cyclopentane-1-carboxamide (190 mg, 346 μmol) as an off-white amorphous solid. LCMS RT 1.129 min, [M+H]⁺ 549, LCMS method C.

### Step 10. Synthesis of (1R,3R)-3-acetamido-N—((S)-(2,3-dichloro-6-fluorophenyl)(1-(2-hydroxyethyl)cyclopentyl)methyl)cyclopentane-1-carboxamide

A mixture of (1R,3R)-3-acetamido-N—((S)-(1-(2-(benzyloxy)ethyl)cyclopentyl)(2,3-dichloro-6-fluorophenyl)methyl)cyclopentane-1-carboxamide (100 mg, 182 μmol) and Ce(NH₄)₂(NO₃)₆ (997 mg, 1.82 mmol) in acetonitrile/H₂O (2:1, 10 mL) was stirred for 16 h at room temperature. The mixture was diluted with water. The mixture was extracted with ethyl acetate (100 mL) three times. The combined organic layers were washed with brine, dried over sodium sulfate, filtered and concentrated in vacuo. The resulting crude material was purified by preparative HPLC (column: Xselect CSH C18 OBD Column 30*150 mm 5 μm; mobile phase A: water (0.1% formic acid), mobile phase B: acetonitrile; flow rate: 60 mL/min; gradient: 36% B to 47% B in 7 min, then 47% B; wavelength: 254/220 nm; RT1 (min): 6.29) to give (1R,3R)-3-acetamido-N—((S)-(2,3-dichloro-6-fluorophenyl)(1-(2-hydroxyethyl)cyclopentyl)methyl)cyclopentane-1-carboxamide (16.3 mg, 35.5 μmol) as an off-white amorphous solid. ¹H NMR (400 MHz, DMSO-d6) δ 8.15 (d, J=8.6 Hz, 1H), 7.77 (d, J=7.0 Hz, 1H), 7.61 (dd, J=9.0, 5.0 Hz, 1H), 7.25 (dd, J=10.8, 8.9 Hz, 1H), 5.51 (d, J=8.5 Hz, 1H), 4.40 (t, J=4.7 Hz, 1H), 4.00 (q, J=6.5 Hz, 1H), 3.43 (s, 2H), 2.99-2.91 (m, 1H), 1.94-1.72 (m, 7H), 1.62 (dt, J=14.0, 8.2 Hz, 3H), 1.56-1.30 (m, 8H), 1.15 (t, J=10.6 Hz, 1H). LCMS RT 0.817 min, [M+H]⁺ 459, LCMS method C.

### Example 25

### (1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide and (1R,3R,4R)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide

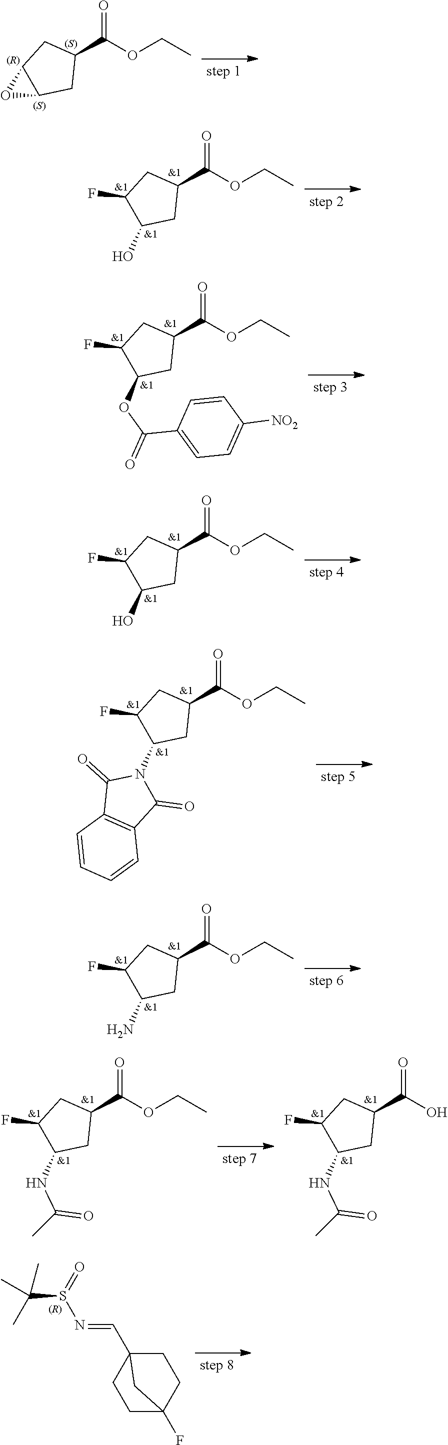

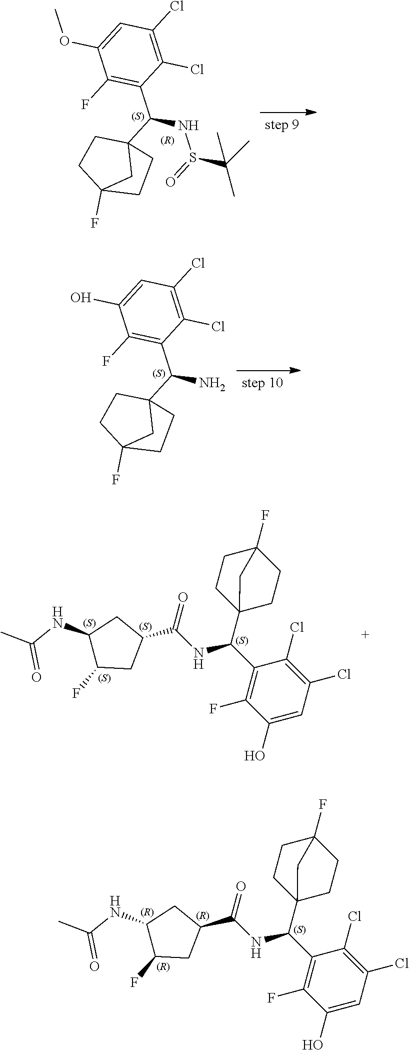

### Step 1. Synthesis of (±)-ethyl (1S,3S,4S)-3-fluoro-4-hydroxycyclopentane-1-carboxylate

A mixture of ethyl (1R,3s,5S)-6-oxabicyclo[3.1.0]hexane-3-carboxylate (9.5 g, 61 mmol) and Et₃N—(HF)₃ (20 g, 0.12 mol) was stirred for 5 h at 110° C. After cooling to room temperature the reaction was quenched by the addition of water (100 mL). The resulting mixture was extracted with ethyl acetate (3×100 mL). The combined organic layers were washed with water (1×100 mL) and dried over anhydrous Na₂SO₄. After filtration, the filtrate was concentrated under reduced pressure. The crude product was purified by silica gel chromatography (120 g column; eluting with petroleum ether/ethyl acetate; ratio: 10/1) to give (±)-ethyl (1S,3S,4S)-3-fluoro-4-hydroxycyclopentane-1-carboxylate (8.0 g, 0.05 mol) as a yellow oil. ¹H NMR (400 MHz, Chloroform-d) δ 4.85 (dddd, J=51.5, 5.6, 3.8, 1.6 Hz, 1H), 4.39 (ddt, J=10.8, 5.2, 2.4 Hz, 1H), 4.16 (q, J=7.1 Hz, 2H), 3.09 (dtd, J=10.0, 8.4, 6.1 Hz, 1H), 2.43 (dddd, J=29.4, 15.4, 10.0, 5.6 Hz, 1H), 2.34-2.08 (m, 2H), 1.97 (ddd, J=14.1, 8.4, 2.7 Hz, 1H), 1.27 (t, J=7.1 Hz, 3H).

### Step 2. Synthesis of (±)-(1R,2S,4S)-4-(ethoxycarbonyl)-2-fluorocyclopentyl 4-nitrobenzoate

To a mixture of (±)-ethyl (1S,3S,4S)-3-fluoro-4-hydroxycyclopentane-1-carboxylate (7.5 g, 43 mmol), 4-nitrobenzoic acid (8.5 g, 51 mmol) and triphenylphosphine (26 g, 98 mmol) was DIAD (20 g, 98 mmol) adde dropwise at 0° C. under N₂. The solution was stirred for 12 h at 25° C. The reaction was quenched by the addition of water (100 mL) at room temperature. The resulting mixture was extracted with ethyl acetate (3×150 mL). The combined organic layers were washed with water (1×100 mL) and dried over anhydrous Na₂SO₄. After filtration, the filtrate was concentrated under reduced pressure. The crude product was purified by silica gel chromatography (120 g column; eluting with petroleum ether/ethyl acetate; ratio: 15/1) to give (±)-(1R,2S,4S)-4-(ethoxycarbonyl)-2-fluorocyclopentyl 4-nitrobenzoate (11.5 g, 35.4 mmol) as a yellow oil. ¹H NMR (400 MHz, DMSO-d6) δ 8.44-8.32 (m, 2H), 8.26-8.13 (m, 2H), 5.37-5.08 (m, 2H), 4.11 (q, J=7.1 Hz, 2H), 3.05 (dtd, J=10.3, 8.5, 6.0 Hz, 1H), 2.40 (tdd, J=22.3, 9.5, 5.7 Hz, 2H), 2.22 (dddd, J=23.6, 15.5, 6.9, 2.7 Hz, 2H), 1.22 (dt, J=14.3, 6.3 Hz, 3H).

### Step 3. Synthesis of (±)-ethyl (1S,3S,4R)-3-fluoro-4-hydroxycyclopentane-1-carboxylate

A mixture of (±)-(1R,2S,4S)-4-(ethoxycarbonyl)-2-fluorocyclopentyl 4-nitrobenzoate (9.5 g, 29 mmol) and lithium hydroxide (0.77 g, 32 mmol) in THF/EtOH/H₂O (30 ml, 4/1/1) was stirred for 2 hours at 25° C. The mixture was concentrated and the aqueous solution's pH was adjusted to 6. The reaction mixture was extracted with ethyl acetate (150 mL) three times. The organic layers were combined, dried over Na₂SO₄ and concentrated. The residue was purified by silica gel chromatography, eluting with petroleum ether/ethyl acetate 3/1 to give (±)-ethyl (1S,3S,4R)-3-fluoro-4-hydroxycyclopentane-1-carboxylate (4 g, 0.02 mol) as a colorless oil. ¹H NMR (400 MHz, Chloroform-d) δ 4.87 (dq, J=54.3, 3.9 Hz, 1H), 4.17 (q, J=7.1 Hz, 2H), 4.11-3.97 (m, 1H), 2.90-2.71 (m, 1H), 2.53 (s, 1H), 2.36 (dt, J=6.2, 3.1 Hz, 3H), 2.31-2.13 (m, 1H), 1.27 (t, J=7.0 Hz, 3H).

### Step 4. Synthesis of (±)-ethyl (1S,3S,4S)-3-(1,3-dioxoisoindolin-2-yl)-4-fluorocyclopentane-1-carboxylate

To a mixture of (±)-ethyl (1S,3S,4R)-3-fluoro-4-hydroxycyclopentane-1-carboxylate (5.7 g, 32 mmol), triphenylphosphane (10 g, 39 mmol) and isoindoline-1,3-dione (5.7 g, 39 mmol) in THF (100 mL) was DIAD (7.9 g, 39 mmol) added dropwise. The solution was stirred for 12 hours at 25° C. The reaction was quenched with water and extracted with ethyl acetate (200 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give (±)-ethyl (1S,3S,4S)-3-(1,3-dioxoisoindolin-2-yl)-4-fluorocyclopentane-1-carboxylate (3 g, 0.01 mol) as a white amorphous solid. ¹H NMR (400 MHz, DMSO-d₆) δ 7.98-7.75 (m, 4H), 5.45 (ddt, J=53.7, 6.6, 4.7 Hz, 1H), 4.70 (dtd, J=24.9, 8.6, 4.4 Hz, 1H), 4.11 (qd, J=7.1, 1.4 Hz, 2H), 3.23 (td, J=8.5, 4.2 Hz, 1H), 2.71-2.50 (m, 1H), 2.36 (ddd, J=14.1, 9.5, 4.8 Hz, 1H), 2.26-2.04 (m, 2H), 1.21 (t, J=7.1 Hz, 3H).

### Step 5. Synthesis of (±)-ethyl (1S,3S,4S)-3-amino-4-fluorocyclopentane-1-carboxylate

A mixture of (±)-ethyl (1S,3S,4S)-3-(1,3-dioxoisoindolin-2-yl)-4-fluorocyclopentane-1-carboxylate (500 mg, 1.64 mmol) and N₂H₄·H₂O (164 mg, 3.28 mmol) in EtOH (20 mL) was stirred for 2 hours at 70° C. The reaction mixture was filtered, the pad was washed with EtOH, and the filtrate was concentrated in vacuo to give (±)-ethyl (1S,3S,4S)-3-amino-4-fluorocyclopentane-1-carboxylate (235 mg, 1.1 mmol) as a yellow oil. LCMS RT 0.481 min, [M+H]⁺ 176, LCMS method B.

### Step 6. Synthesis of (±)-ethyl (1S,3S,4S)-3-acetamido-4-fluorocyclopentane-1-carboxylate

To a mixture of (±)-ethyl (1S,3S,4S)-3-amino-4-fluorocyclopentane-1-carboxylate (235 mg, 1.34 mmol) and triethylamine (407 mg, 4.02 mmol) in DCM (5 mL) was added acetyl chloride (158 mg, 2.01 mmol) dropwise. The solution was stirred for 2 hours at 0° C. The reaction was quenched with water (10 mL) and extracted with ethyl acetate (50 mL*3). The combined organic layers were washed with brine, dried over sodium sulfate, filtered and concentrated in vacuo to give (±)-ethyl (1S,3S,4S)-3-acetamido-4-fluorocyclopentane-1-carboxylate (200 mg, 921 μmol) as a yellow oil. LCMS RT 0.542 min, [M+H]⁺ 218, LCMS method C.

### Step 7. Synthesis of (±)-(1S,3S,4S)-3-acetamido-4-fluorocyclopentane-1-carboxylic acid

A mixture of (±)-ethyl (1S,3S,4S)-3-acetamido-4-fluorocyclopentane-1-carboxylate (240 mg, 1.10 mmol) and LiOH (79.4 mg, 3.31 mmol) was dissolved in MeOH/H₂O (4 ml, 3/1). The solution was stirred at 25° C. for 3 hours. The mixture was concentrated and the residue's pH was adjusted to 6. The solution was concentrated in vacuo to give (±)-(1S,3S,4S)-3-acetamido-4-fluorocyclopentane-1-carboxylic acid (200 mg, 1.06 mmol) as a white amorphous solid, which was used in the next step without purification. LCMS RT 0.278 min, [M+H]⁺ 190, LCMS method A.

### Step 8. Synthesis of (R)—N—((S)-(2,3-dichloro-6-fluoro-5-methoxyphenyl) (4-fluorobicyclo[2.2.1]heptan-1-yl)methyl)-2-methylpropane-2-sulfinamide

To a solution of 1,2-dichloro-4-fluoro-5-methoxybenzene (1 g, 6 mmol) in THF (80 mL) was added LDA (2 M in THF, 5.5 mL, 11 mmol) dropwise at −78° C. under a N₂ atmosphere. The reaction mixture was stirred at −78° C. for 1 hour prior to the addition of a solution of (R)—N-((4-fluorobicyclo[2.2.1]heptan-1-yl)methylene)-2-methylpropane-2-sulfinamide (1 g, 4 mmol) in THF (5 mL) at −78° C. under N₂. The mixture was stirred for 2 hours at −78° C. The reaction was quenched with saturated NH₄Cl solution (100 mL), and the mixture was extracted with EtOAc (3*100 mL). The combined organic extracts were washed with brine (100 mL) and dried over anhydrous Na₂SO₄. The resulting crude material was purified by flash chromatography (acetonitrile/water) to give (R)—N—((S)-(2,3-dichloro-6-fluoro-5-methoxyphenyl) (4-fluorobicyclo[2.2.1]heptan-1-yl)methyl)-2-methylpropane-2-sulfinamide (880 mg, 2.00 mmol) as a yellow oil. LCMS RT 1.10 min, [M+H]⁺ 440, LCMS method C.

### Step 9. Synthesis of (S)-3-(amino(4-fluorobicyclo[2.2.1]heptan-1-yl)methyl)-4,5-dichloro-2-fluorophenol

To (R)—N—((S)-(2,3-dichloro-6-fluoro-5-methoxyphenyl) (4-fluorobicyclo[2.2.1]heptan-1-yl)methyl)-2-methylpropane-2-sulfinamide (940 mg, 2.13 mmol) was added HBr (40 ml, 33% in AcOH). The solution was stirred for 24 hours at 100° C. The resulting mixture was concentrated under reduced pressure. The mixture was adjusted to pH 7 with NaOH (4 N, aq.). The resulting mixture was extracted with ethyl acetate (3×50 mL). The combined organic layers were washed with water (50 mL) and dried over anhydrous Na₂SO₄. After filtration, the filtrate was concentrated under reduced pressure. The resulting crude material was purified by flash chromatography (acetonitrile/water) to give (S)-3-(amino(4-fluorobicyclo[2.2.1]heptan-1-yl)methyl)-4,5-dichloro-2-fluorophenol (630 mg, 1.96 mmol) as a colorless oil. LCMS RT 0.66 min, [M+H]⁺ 322, LCMS method D.

### Step 10. Synthesis of (1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide and (1R,3R,4R)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide

To a mixture of (S)-3-(amino(4-fluorobicyclo[2.2.1]heptan-1-yl)methyl)-4,5-dichloro-2-fluorophenol (50 mg, 0.16 mmol), (±)-(1S,3S,4S)-3-acetamido-4-fluorocyclopentane-1-carboxylic acid (29 mg, 0.16 mmol) and NaHCO₃ (39 mg, 0.47 mmol) in DMF (1 mL) was added HATU (88 mg, 0.23 mmol). The mixture was stirred for 1 h at 25° C. The reaction mixture was diluted with water (50 mL), and the aqueous phase was extracted with ethyl acetate (50 ml) three times. The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; 0% to 100% gradient in 10 min; detector: UV 220 nm. The resulting crude material was purified by chiral preparative HPLC (column: Sunfire prep C18 column, 30*150 mm, 5 μm; mobile phase A: water (0.1% formic acid), mobile phase B: acetonitrile; flow rate: 60 mL/min; gradient: 40% B to 51% B in 7 min, then 51% B; wavelength: 254/220 nm; RT1 (min): 6.5) to give (±)-(1S,3S,4S)-3-acetamido-N—((S)-(2,3-dichloro-6-fluoro-5-hydroxyphenyl)(4-fluorobicyclo[2.2.1]heptan-1-yl)methyl)-4-fluorocyclopentane-1-carboxamide (40 mg, 81 μmol) as an off-white solid.

The product was further purified by chiral preparative HPLC (column: CHIRALPAK IE, 2*25 cm, 5 μm; mobile phase A: hexane (0.5% 2 M NH₃-MeOH), mobile phase B: EtOH; flow rate: 20 mL/min; gradient: 20% B isocratic; wavelength: 220/254 nm; RT1 (min): 6.18; RT2 (min): 7.67; sample solvent: EtOH; injection volume: 0.35 mL) to give (1S,3S,4S)-3-acetamido-N—((S)-(2,3-dichloro-6-fluoro-5-hydroxyphenyl) (4-fluorobicyclo[2.2.1]heptan-1-yl)methyl)-4-fluorocyclopentane-1-carboxamide and (1R,3R,4R)-3-acetamido-N—((S)-(2,3-dichloro-6-fluoro-5-hydroxyphenyl) (4-fluorobicyclo[2.2.1]heptan-1-yl)methyl)-4-fluorocyclopentane-1-carboxamide, both as an off-white amorphous solid.

Isomer 1: 5.2 mg, 10 μmol. ¹H NMR (400 MHz, DMSO-d6) δ 8.14 (d, J=8.4 Hz, 1H), 7.93 (d, J=6.9 Hz, 1H), 7.08 (d, J=8.2 Hz, 1H), 5.51-5.35 (m, 1H), 4.83 (dq, J=53.3, 5.0 Hz, 1H), 4.07 (d, J=11.8 Hz, 1H), 3.00 (p, J=7.9 Hz, 1H), 2.34-2.15 (m, 1H), 1.99 (dt, J=14.0, 7.7 Hz, 1H), 1.80 (d, J=4.6 Hz, 6H), 1.75-1.61 (m, 5H), 1.60-1.35 (m, 4H). LCMS RT 0.958 min, [M+H]⁺ 493.15, LCMS method B.

Isomer 2: 5.7 mg, 11 μmol. ¹H NMR (400 MHz, DMSO-d6) b 10.60 (s, 1H), 8.36-8.11 (m, 1H), 7.93 (d, J=6.7 Hz, 1H), 7.11 (d, J=8.2 Hz, 1H), 5.51-5.29 (m, 1H), 4.86 (dq, J=53.2, 4.6 Hz, 1H), 4.28-3.97 (m, 1H), 3.08-2.89 (m, 1H), 2.39-2.24 (m, 1H), 2.04-1.83 (m, 4H), 1.80 (s, 5H), 1.74-1.60 (m, 4H), 1.54 (dq, J=21.6, 10.1, 9.0 Hz, 3H). LCMS RT 1.522 min, [M+H]⁺ 493.10, LCMS method B.

### Example 26

### (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide

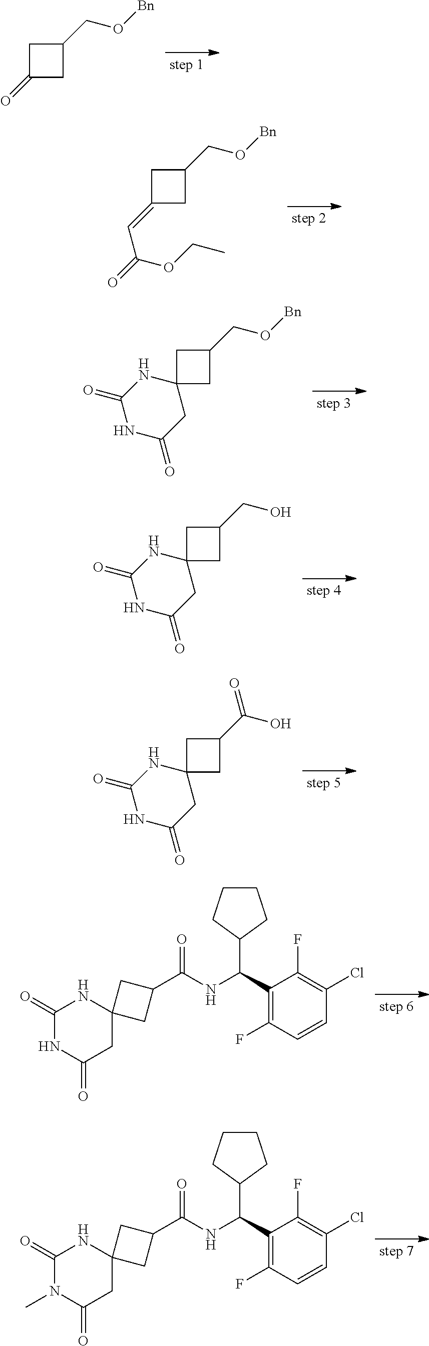

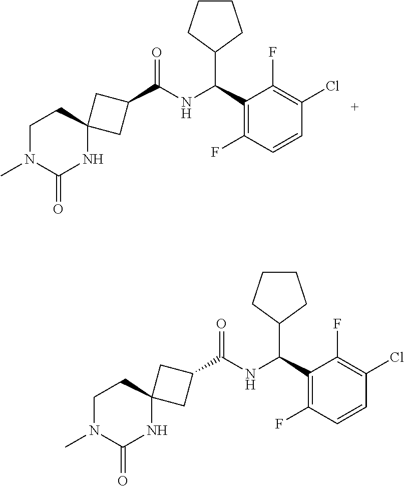 [SMILES: CN1CC[C@]2(C[C@@H](C(=O)N[C@H](c3c(F)ccc(Cl)c3F)C3CCCC3)C2)NC1=O ; CN1CC[C@]2(C[C@H](C(=O)N[C@H](c3c(F)ccc(Cl)c3F)C3CCCC3)C2)NC1=O]

### Step 1. Synthesis of ethyl 2-(3-((benzyloxy)methyl)cyclobutylidene)acetate

To a mixture of 3-((benzyloxy)methyl)cyclobutan-1-one (10 g, 53 mmol) and ethyl 2-(diethoxyphosphoryl)acetate (14 g, 63 mmol) in THF (100 mL) was added NaH (1.3 g, 53 mmol) in portions at 0° C. The mixture was stirred for 1 hour at room temperature. The reaction was quenched with saturated NH₄Cl (aq.) (30 ml) and the aqueous phase was extracted with ethyl acetate (100 mL) three times. The combined organic layers were washed with brine, dried over sodium sulfate, filtered and concentrated in vacuo. The resulting crude material was purified by silica gel chromatography (100 g column; eluting with petroleum ether:ethyl acetate 5:1) to give ethyl 2-(3-((benzyloxy)methyl)cyclobutylidene)acetate (13.46 g, 51.70 mmol) as a colorless oil. LCMS RT 1.082 min, [M+H]n 261, LCMS method C.

### Step 2. Synthesis of 2-((benzyloxy)methyl)-5,7-diazaspiro[3.5]nonane-6,8-dione

To a mixture of ethyl 2-(3-((benzyloxy)methyl)cyclobutylidene)acetate (11 g, 42 mmol) and urea (15 g, 0.2 mol) in NMP (120 mL) was added DBU (25 mL, 0.17 mol) at room temperature. The mixture was stirred for 16 h at 140° C. After cooling to room temperature, the reaction mixture was diluted with water (150 mL), and the aqueous phase was extracted with ethyl acetate (150 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 60% B in 10 min; detector: UV 220 nm) to give 2-((benzyloxy)methyl)-5,7-diazaspiro[3.5]nonane-6,8-dione (7.07 g, 25.8 mmol) as a yellow oil. LCMS RT 0.902 min, [M+H]⁺ 275, LCMS method C.

### Step 3. Synthesis of 2-(hydroxymethyl)-5,7-diazaspiro[3.5]nonane-6,8-dione

A mixture of 2-((benzyloxy)methyl)-5,7-diazaspiro[3.5]nonane-6,8-dione (5.5 g, 20 mmol) and Pd/C (0.21 g) in MeOH (60 mL) was treated with H₂ (20 atm) and stirred at room temperature overnight. The reaction mixture was filtered through a pad of Celite, the pad was washed with MeOH (200 mL), and the filtrate was concentrated in vacuo. The resulting crude material was purified by silica gel chromatography (100 g column; eluting with DCM:MeOH 25:1) to give 2-(hydroxymethyl)-5,7-diazaspiro[3.5]nonane-6,8-dione (3.22 g, 17.5 mmol) as a white solid. LCMS RT 0.202 min, [M+H]⁺ 185, LCMS method C.

### Step 4. Synthesis of 6,8-dioxo-5,7-diazaspiro[3.5]nonane-2-carboxylic acid

To a mixture of 2-(hydroxymethyl)-5,7-diazaspiro[3.5]nonane-6,8-dione (400 mg, 2.17 mmol) in H₂O (5 mL) was added a solution of KMnO₄ (343 mg, 2.17 mmol) in H₂O (5 mL) at 0° C. The mixture was stirred for 3 hours at room temperature. The reaction mixture was filtered through a pad of Celite, the pad was washed with MeOH (50 mL), and the filtrate was concentrated in vacuo to afford 6,8-dioxo-5,7-diazaspiro[3.5]nonane-2-carboxylic acid (400 mg, 2.02 mmol) as a brown solid. LCMS RT 0.119 min, [M+H]⁺ 199, LCMS method D.

### Step 5. Synthesis of (S)—N-((3-chloro-2,6-difluorophenyl)(cyclopentyl)methyl)-6,8-dioxo-5,7-diazaspiro[3.5]nonane-2-carboxamide

To a mixture of 6,8-dioxo-5,7-diazaspiro[3.5]nonane-2-carboxylic acid (400 mg, 2.02 mmol), (S)-(3-chloro-2,6-difluorophenyl) (cyclopentyl)methanamine (496 mg, 2.02 mmol) and TEA (612 mg, 6.0 m mol) in DMF (4 mL) was added T₃P (1.93 g, 6.06 mmol) at room temperature. The mixture was stirred for 1 h at room temperature. The reaction mixture was diluted with water (20 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XB ridge Prep OBD C18 Column, 30*150 mm, 5 μm; mobile phase A: water (10 mM NH₄HCO₃), mobile phase B: acetonitrile; flow rate: 60 mL/min; gradient: 25% B to 55% B in 8 min, then 55% B; wavelength: 220 nm; RT1 (min): 7.68) to give (S)—N-((3-chloro-2,6-difluorophenyl)(cyclopentyl)methyl)-6,8-dioxo-5,7-diazaspiro[3.5]nonane-2-carboxamide[3.5]nonane-2-carboxamide (380 mg, 892 μmol) as a white amorphous solid. LCMS RT 1.060 min, [M+H]⁺ 390, LCMS method C.

### Step 6. Synthesis of (S)—N-((3-chloro-2,6-difluorophenyl)(cyclopentyl)methyl)-7-methyl-6,8-dioxo-5,7-diazaspiro[3.5]nonane-2-carboxamide

To a mixture of (S)—N-((3-chloro-2,6-difluorophenyl) (cyclopentyl)methyl)-6,8-dioxo-5,7-diazaspiro[3.5]nonane-2-carboxamide (140 mg, 329 μmol) in toluene (2 mL) was added 1,1-dimethoxy-N,N-dimethylethan-1-amine (131 mg, 986 μmol). The mixture was stirred for 2 h at 110° C. The mixture was concentrated in vacuo. The residue was purified by reverse phase flash chromatography (column: C18 silica gel; mobile phase A: water, mobile phase B: acetonitrile; gradient: 45% to 65% B in 15 min; detector: UV 220 nm) to give (S)—N-((3-chloro-2,6-difluorophenyl)(cyclopentyl)methyl)-7-methyl-6,8-dioxo-5,7-diazaspiro[3.5]nonane-2-carboxamide (100 mg, 227 μmol) as a colorless oil. LCMS RT 1.135 min, [M+H]⁺ 440, LCMS method C.

### Step 7. Synthesis of (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide

To a mixture of (S)—N-((3-chloro-2,6-difluorophenyl) (cyclopentyl)methyl)-7-methyl-6,8-dioxo-5,7-diazaspiro[3.5]nonane-2-carboxamide (80 mg, 0.18 mmol) in THF (2 mL) were added BF₃-Et₂O (31 mg, 0.22 mmol) and NaBH₄ (6.9 mg, 0.18 mmol) at 0° C. The mixture was stirred for 1 h at room temperature. The reaction mixture was diluted with water (10 mL), and the aqueous phase was extracted with ethyl acetate (10 mL) three times. The combined organic layers were washed with brine, dried over sodium sulfate, filtered and concentrated in vacuo. The resulting crude material was purified by preparative HPLC (column: YMC-Actus Triart C18 Ex RS, 30*150 mm, 5 μm; mobile phase A: water (10 mM NH₄HCO₃+0.1% NH₄OH), mobile phase B: acetonitrile; flow rate: 60 mL/min; gradient: 30% B to 55% B in 9 min, 55% B to 60% B in 9.5 min, then 60% B; wavelength: 220 nm; RT1 (min): 9.13) to give (S)—N-((3-chloro-2,6-difluorophenyl) (cyclopentyl)methyl)-7-methyl-6-oxo-5,7-diazaspiro[3.5]nonane-2-carboxamide (35 mg, 82 μmol) as an off-white solid. LCMS RT 1.503 min, [M+H]⁺ 426, LCMS method D.

The product was purified by preparative chiral HPLC (column: DZ-CHIRALPAK IC-3, 4.6*50 mm, 3.0 μm; mobile phase A: hexane:EtOH 70:30; flow rate: 1 mL/min; gradient: 0% B isocratic; injection volume: 0.5 mL). Lyophilization yielded (2r,4S)—N—((S)-(3-chloro-2,6-difluorophenyl)(cyclopentyl)methyl)-7-methyl-6-oxo-5,7-diazaspiro[3.5]nonane-2-carboxamide and (2s,4R)—N—((S)-(3-chloro-2,6-difluorophenyl)(cyclopentyl)methyl)-7-methyl-6-oxo-5,7-diazaspiro[3.5]nonane-2-carboxamide, both as an off-white amorphous solid.

Isomer 1: 2.3 mg, 5.4 μmol. ¹H NMR (400 MHz, DMSO-d₆) δ 8.37 (d, J=7.4 Hz, 1H), 7.53 (td, J=8.7, 5.5 Hz, 1H), 7.12 (t, J=9.6 Hz, 1H), 6.70 (s, 1H), 4.82 (dd, J=11.1, 7.4 Hz, 1H), 3.04 (t, J=6.0 Hz, 2H), 2.94 (dq, J=10.0, 4.9 Hz, 1H), 2.71 (s, 3H), 2.41 (d, J=9.1 Hz, 1H), 2.25 (t, J=11.0 Hz, 1H), 2.15 (q, J=14.3, 12.8 Hz, 2H), 2.03 (d, J=12.6 Hz, 1H), 1.90 (d, J=8.2 Hz, 1H), 1.74 (dd, J=7.4, 4.7 Hz, 2H), 1.59 (s, 3H), 1.51 (dt, J=16.6, 9.3 Hz, 1H), 1.38-1.30 (m, 1H), 1.24 (s, 1H), 1.00 (s, 1H). LCMS RT 1.537 min, [M+H]⁺ 426, LCMS method D;

Isomer 2: 3.1 mg, 7.3 μmol. ¹H NMR (400 MHz, DMSO-d₆) δ 8.27 (d, J=7.5 Hz, 1H), 7.53 (td, J=8.7, 5.5 Hz, 1H), 7.12 (t, J=9.3 Hz, 1H), 6.54 (s, 1H), 4.83 (dd, J=11.2, 7.5 Hz, 1H), 3.14 (t, J=5.9 Hz, 2H), 2.72 (s, 3H), 2.41 (d, J=9.0 Hz, 1H), 2.26 (t, J=11.0 Hz, 1H), 2.16 (t, J=10.1 Hz, 1H), 2.11-2.01 (m, 2H), 2.00 (s, 1H), 1.94-1.85 (m, 1H), 1.80 (t, J=5.9 Hz, 2H), 1.64-1.51 (m, 4H), 1.49 (dd, J=15.5, 7.5 Hz, 1H), 1.38-1.30 (m, 1H), 1.00 (s, 1H). LCMS RT 1.537 min, [M+H]⁺ 426, LCMS method D.

### Example 27

### (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

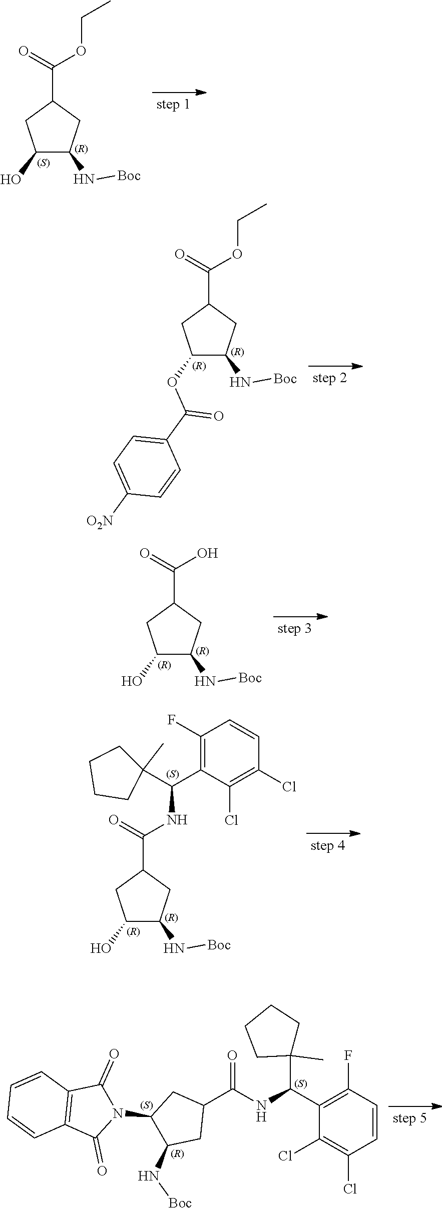

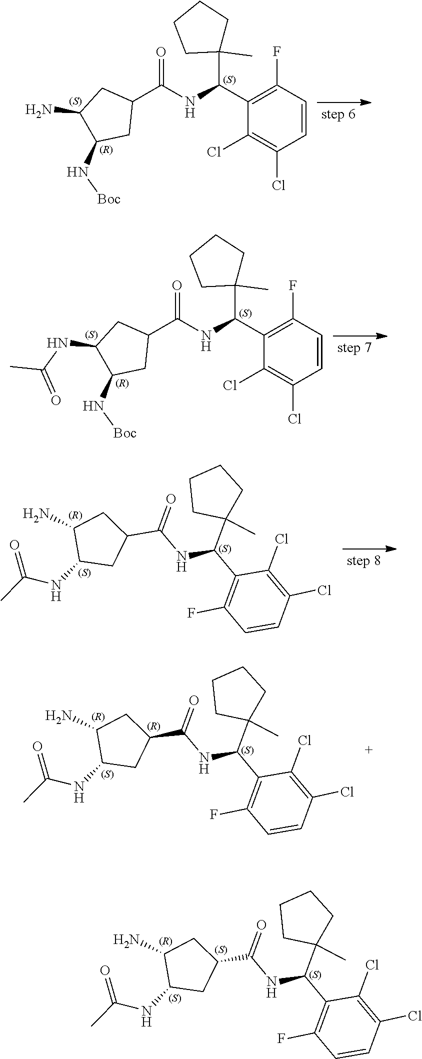

### Step 1. Synthesis of (1R,2R)-2-((tert-butoxycarbonyl)amino)-4-(ethoxycarbonyl)cyclopentyl 4-nitrobenzoate

To a stirred solution of ethyl (3R,4S)-3-((tert-butoxycarbonyl)amino)-4-hydroxycycl opentane-1-carboxylate (1.91 g, 7 mmol), 4-nitrobenzoic acid (1.17 g, 7 mmol) and tripheny lphosphine (1.83 g, 7 mmol) in THF (20 mL) was added DIAD (1.41 g, 7 mmol) dropwise a t 0° C. The resulting mixture was stirred for 2 h at 25° C. The reaction mixture was diluted with water (30 mL), and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 100% B in 30 min; detector: UV 254 nm) to afford (1R,2R)-2-((tert-butoxycarbonyl)amino)-4-(ethoxycarbonyl)cyclopentyl 4-nitrobenzoate (1.2 g, 2.8 mmol) as a white solid. LCMS RT=1.23 min, [M+H]⁺ 423, LCMS method A.

### Step 2. Synthesis of (3R,4R)-3-((tert-butoxycarbonyl)amino)-4-hydroxycyclopentane-1-carboxylic acid

To a solution of (1R,2R)-2-((tert-butoxycarbonyl)amino)-4-(ethoxycarbonyl)cyclo pentyl 4-nitrobenzoate (500 mg, 1.18 mmol) in MeOH (4 mL) was added lithium hydroxide (142 mg, 5.92 mmol) in H₂O (1 mL). The mixture was stirred at 25° C. for 1 hour. The solution was concentrated under reduced pressure to remove MeOH. The residue was acidified to pH 5-6 with HCl (2 N). The solution was concentrated to dryness under reduced pressure to give (3R,4R)-3-((tert-butoxycarbonyl)amino)-4-hydroxycyclopentane-1-carboxylic acid (270 mg, 1.10 mmol) as a white amorphous solid. LCMS RT 0.388 min, [M−H]⁻ 244, LCMS method B.

### Step 3. Synthesis of tert-butyl ((1R,2R)-4-(((S)-(2,3-dichloro-6-fluorophenyl) (1-methylcyclopentyl)methyl) carbamoyl)-2-hydroxycyclopentyl) carbamate

To a mixture of (3R,4R)-3-((tert-butoxycarbonyl)amino)-4-hydroxycyclopentane-1-carboxylic acid (270 mg, 1.10 mmol), (S)-(2,3-dichloro-6-fluorophenyl) (1-methylcyclopent yl) methanamine (365 mg, 1.32 mmol) and NaHCO₃ (370 mg, 4.40 mmol) in DMF (5 mL) was added HATU (837 mg, 2.20 mmol). The mixture was stirred at room temperature for 1 hour. The reaction was quenched with water (10 ml) and extracted with ethyl acetate (20 ml*3). The combined organic layers were washed with brine, dried over Na₂SO₄ and concentra ted. The residue was purified by reverse phase flash chromatography (column: C18 silica gel; mobile phase A: water, mobile phase B: acetonitrile; gradient: 10% to 50% B in 10 min; detector: UV 254 nm) to give tert-butyl ((1R,2R)-4-(((S)-(2,3-dichloro-6-fluorophenyl) (1-methylcyclopentyl)methyl) carbamoyl)-2-hydroxycyclopentyl) carbamate (300 mg, 596 μmol) as a yellow oil. LCMS RT 1.129 min, m/z [M−56+H]⁺446, LCMS method C.

### Step 4. Synthesis of tert-butyl ((1R,2S)-4-(((S)-(2,3-dichloro-6-fluorophenyl)(1-methylcyclopentyl)methyl)carbamoyl)-2-(1,3-dioxoisoindolin-2-yl)cyclopentyl)carbamate

To a mixture of tert-butyl ((1R,2R)-4-(((S)-(2,3-dichloro-6-fluorophenyl) (1-methylcyclopentyl)methyl) carbamoyl)-2-hydroxycyclopentyl) carbamate (105 mg, 715 μmol) and triphenylphosphine (234 mg, 894 μmol) in THF (6 mL) was added DIAD (174 μL, 894 μmol) dropwise at 0° C. under a nitrogen atmosphere. The mixture was stirred for 16 hours at 25° C. The reaction mixture was diluted with water (10 mL), and the aqueous phase was extract ed with ethyl acetate (40 mL) three times. The combined organic layers were washed with b rine, dried over sodium sulfate, filtered and concentrated in vacuo. The residue was purified by reverse phase flash chromatography (column: C18 silica gel; mobile phase A: water, mob ile phase B: acetonitrile; gradient: 0% to 100% B in 20 min; detector: UV 254 nm) to give tert-butyl ((1R,2S)-4-(((S)-(2,3-dichloro-6-fluorophenyl) (1-methylcyclopentyl)methyl) carba moyl)-2-(1,3-dioxoisoindolin-2-yl)cyclopentyl) carbamate (240 mg, 379 μmol) as a yellow oil. LCMS RT 1.279 min, [M−56+H]⁺576, LCMS method C.

### Step 5. Synthesis of tert-butyl ((1R,2S)-2-amino-4-(((S)-(2,3-dichloro-6-fluorophenyl)(1-methylcyclopentyl)methyl)carbamoyl)cyclopentyl)carbamate

To a solution of tert-butyl ((1R,2S)-4-(((S)-(2,3-dichloro-6-fluorophenyl) (1-methyl cyclopentyl)methyl) carbamoyl)-2-(1,3-dioxoisoindolin-2-yl)cyclopentyl) carbamate (220 mg, 348 μmol) in EtOH (4 mL) was added hydrazine hydrate (34.8 mg, 696 μmol). The mixture was heated at 70° C. for 2 hours. The reaction mixture was filtered, the collected solid w as washed with EtOH, and the filtrate was concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give ter t-butyl ((1R,2S)-2-amino-4-(((S)-(2,3-dichloro-6-fluorophenyl)(1-methylcyclopentyl)methyl)carbamoyl)cyclopentyl)carbamate (130 mg, 259 μmol) as a white amorphous solid. LCMS RT 1.035 min, [M+H]⁺ 502, LCMS method C.

### Step 6. Synthesis of tert-butyl ((1R,2S)-2-acetamido-4-(((S)-(2,3-dichloro-6-fluorophenyl)(1-methylcyclopentyl)methyl)carbamoyl)cyclopentyl)carbamate

To a mixture of tert-butyl ((1R,2S)-2-amino-4-(((S)-(2,3-dichloro-6-fluorophenyl)(1-methylcyclopentyl)methyl)carbamoyl)cyclopentyl)carbamate (120 mg, 239 μmol), acetic acid (17.2 mg, 287 μmol) and NaHCO₃ (80.2 mg, 955 μmol) in DMF (4 mL) was added HATU (182 mg, 478 μmol). The mixture was stirred at room temperature for 1 hour. The reaction was quenched with water (10 ml) and extracted with ethyl acetate (20 mL*3). The combined organic layers were washed with brine, dried over Na₂SO₄ and concentrated. The residue w as purified by reverse phase flash chromatography (column: C18 silica gel; mobile phase A: water, mobile phase B: acetonitrile; gradient: 10% to 50% B in 10 min; detector: UV 254 n m) to give tert-butyl ((1R,2S)-2-acetamido-4-(((S)-(2,3-dichloro-6-fluorophenyl)(1-methylcyclopentyl)methyl)carbamoyl)cyclopentyl)carbamate (100 mg, 184 μmol) as a yellow oil. L CMS RT 1.238 min, [M−100+H]⁺444, LCMS method B.

### Step 7. Synthesis of (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

A solution of tert-butyl ((1R,2S)-2-acetamido-4-(((S)-(2,3-dichloro-6-fluorophenyl)(1-methylcyclopentyl)methyl)carbamoyl)cyclopentyl)carbamate (100 mg, 184 μmol) in HCl (4 mL, 4 N in MeOH) was stirred at 25° C. for 1 hour. The solution was concentrated under r educed pressure. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-4-amino-N—((S)-(2,3-dichloro-6-fluorophenyl) (1-methylcyclopentyl)methyl)cyclopentane-1-carboxamide (50 mg, 0.11 mmol) as a white amorphous solid. LCMS RT 0.927 min, [M+H]⁺ 444, LCMS method C.

### Step 8. Synthesis of (1R,3S,4R)-3-acetamido-4-amino-N—((S)-(2,3-dichloro-6-fluorophenyl)(1-methylcyclopentyl)methyl)cyclopentane-1-carboxamide and (1S,3S,4R)-3-acetamido-4-amino-N—((S)-(2,3-dichloro-6-fluorophenyl)(1-methylcyclopentyl)methyl)cyclopentane-1-carboxamide

(3S,4R)-3-acetamido-4-amino-N—((S)-(2,3-dichloro-6-fluorophenyl)(1-methylcyclop entyl)methyl)cyclopentane-1-carboxamide (50 mg, 0.11 mmol) was purified by chiral prepa rative HPLC (column: CHIRALPAK IH, 2*25 cm, 5 μm; mobile phase A: hexane (0.5% 2 M NH₃ in MeOH), mobile phase B: EtOH:DCM 1:1; flow rate: 20 mL/min; gradient: 15% B isocratic; wavelength: 220/254 nm; RT1 (min): 6.23; RT2 (min): 7.94; sample solvent: EtOH:DCM 1:1; injection volume: 0.25 mL) to give (1R,3S,4R)-3-acetamido-4-amino-N—((S)-(2,3-dichloro-6-fluorophenyl)(1-methylcyclopentyl)methyl)cyclopentane-1-carboxamid e and (1S,3S,4R)-3-acetamido-4-amino-N—((S)-(2,3-dichloro-6-fluorophenyl)(1-methylcyclopentyl)methyl)cyclopentane-1-carboxamide, both as a white amorphous solid.

Isomer 1: 5.7 mg, 13 μmol. ¹H NMR (400 MHz, DMSO-d₆) δ 8.08 (d, J=8.9 Hz, 1H), 7.59 (s, 2H), 7.25 (t, J=9.9 Hz, 1H), 5.47 (d, J=8.5 Hz, 1H), 3.90 (s, 1H), 3.31 (s, 1H), 3.12-3.04 (m, 2H), 2.31 (s, 1H), 1.82 (d, J=6.8 Hz, 3H), 1.59 (s, 9H), 1.37 (s, 1H), 1.23 (s, 3H), 0.96 (s, 3H). LCMS RT 1.298 min, [M+H]⁺ 444.15, LCMS method C

Isomer 2: 4.8 mg, 11 μmol. ¹H NMR (400 MHz, DMSO-d₆) δ 8.10 (s, 1H), 7.61 (dd, J=9.0, 4.9 Hz, 2H), 7.25 (t, J=9.9 Hz, 1H), 5.46 (d, J=8.4 Hz, 1H), 3.89 (s, 1H), 1.82 (s, 3H), 1.70 (d, J=9.6 Hz, 4H), 1.59 (s, 9H), 1.36 (d, J=10.6 Hz, 1H), 1.25 (d, J=11.8 Hz, 2H), 0.99-0.93 (m, 3H). LCMS RT 0.938 min, [M+H]⁺ 444, LCMS method D.

### Example 28

### (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide

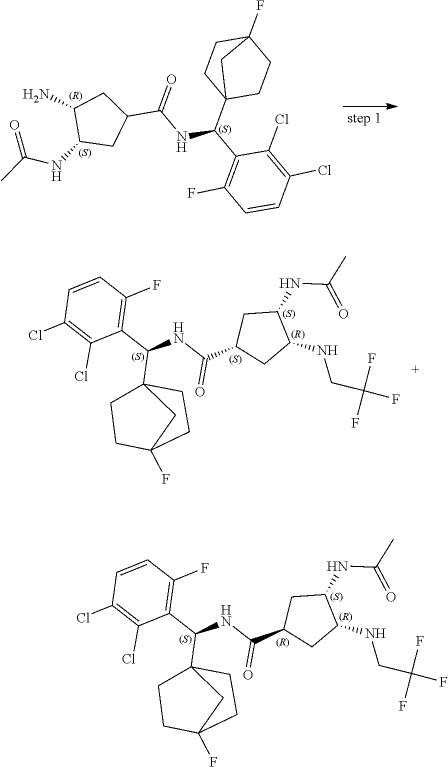

### Step 1. Synthesis of (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide

To a mixture of (3S,4R)-3-acetamido-4-amino-N—((S)-(2,3-dichloro-6-fluorophenyl)(4-fluorobicyclo[2.2.1]heptan-1-yl)methyl)cyclopentane-1-carboxamide (70 mg, 0.15 mmol) and 4 Å molecular sieves (200 mg) in MeOH (4 mL) was added 2,2,2-trifluoroacetaldehyde (22 mg, 0.22 mmol). The mixture was stirred at 25° C. for 30 min prior to the addition of NaBH₃CN (28 mg, 0.44 mmol). The mixture was stirred for 16 hours at 25° C. The reaction mixture was diluted with water (5 mL), and the aqueous phase was extracted with ethyl acetate (10 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give a yellow oil, which w as further purified by chiral preparative HPLC (column: (R, R)-WHELK-O1-Kromasil, 2.11*25 cm, 5 μm; mobile phase A: hexane (0.5% 2 M NH₃ in MeOH), mobile phase B: isoprop-anol:DCM 1:1; flow rate: 20 mL/min; gradient: 40% B isocratic; wavelength: 220/254 nm RT1 (min): 14.62; RT2 (min): 22.08; sample solvent: EtOH:DCM 1:1; injection volume: 0.7 mL) to give (1S,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide a nd (1R,3S,4R)-3-acetamido-N—((S)-(2,3-dichloro-6-fluorophenyl)(4-fluorobicyclo[2.2.1]heptan-1-yl)methyl)-4-((2,2,2-trifluoroethyl)amino)cyclopentane-1-carboxamide, both as a whit e amorphous solid.

Isomer 1: 10 mg, 0.022 mmol. LCMS RT 1.078 mm, [M+H]⁺ 556, LCMS method D. ¹H NMR (400 MHz, DMSO-d₆) δ 8.13 (d, J=8.2 Hz, 1H), 7.61 (dd, J=8.9, 5.0 Hz, 1H), 7.48 (d, J=7.4 Hz, 1H), 7.25 (dd, J=10.7, 9.0 Hz, 1H), 5.48 (d, J=7.9 Hz, 1H), 4.12-3.84 (m, 1H), 3.28-3.11 (m, 3H), 2.96 (d, J=6.3 Hz, 1H), 2.11 (q, J=7.4 Hz, 1H), 1.95-1.43 (m, 16H).

Isomer 2: 7 mg, 0.016 mmol. LCMS RT 1.078 min, [M+H]⁺ 556, LCMS method D. ¹H NMR (400 MHz, DMSO-d₆) δ 8.14 (d, J=8.2 Hz, 1H), 7.62 (dd, J=9.0, 5.1 Hz, 1H), 7.49 (d, J=7.5 Hz, 1H), 7.26 (dd, J=10.6, 9.0 Hz, 1H), 5.50 (d, J=8.1 Hz, 1H), 4.06 (p, J=6.0 Hz, 1H), 3.24-3.04 (m, 1H), 3.00-2.92 (m, 3H), 2.09 (q, J=7.3 Hz, 1H), 1.84 (s, 4H), 1.91-1.75 (m, 4H), 1.68 (qd, J=18.8, 17.1, 7.3 Hz, 5H), 1.57 (d, J=8.1 Hz, 2H), 0.06 (s, 2H)

### Example 29

### (1R,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide

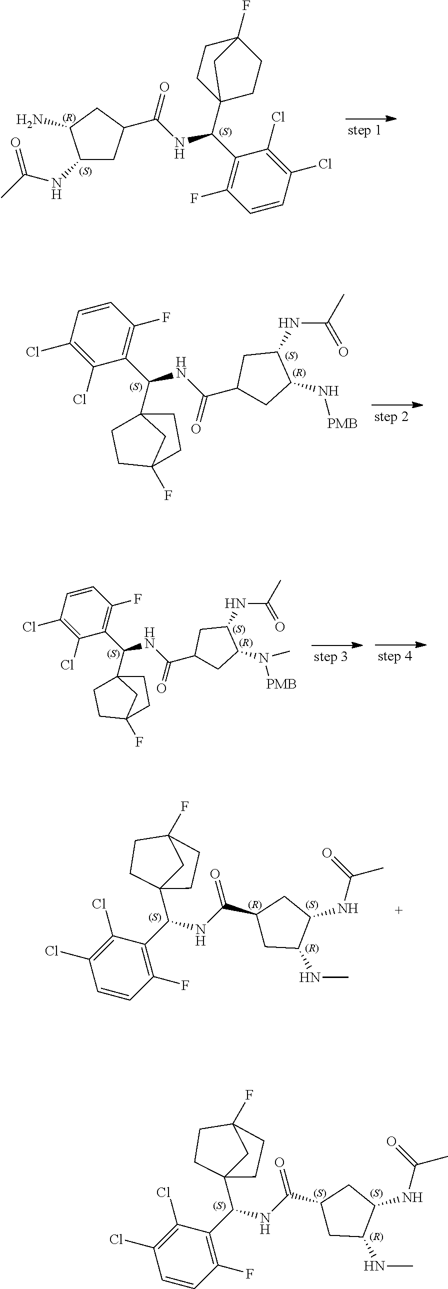

### Step 1. Synthesis of (3S,4R)-3-acetamido-N—((S)-(2,3-dichloro-6-fluorophenyl)(4-fluorobicyclo[2.2.1]heptan-1-yl)methyl)-4-((4-methoxybenzyl)amino)cyclopentane-1-carboxamide

To a mixture of (3S,4R)-3-acetamido-4-amino-N—((S)-(2,3-dichloro-6-fluorophenyl)(4-fluorobicyclo[2.2.1]heptan-1-yl)methyl)cyclopentane-1-carboxamide (70 mg, 0.15 mmol) in MeOH (5 mL) was added 4-methoxybenzaldehyde (30 mg, 0.22 mmol). The mixture was stirred at room temperature for 2 hours prior to the addition of NaBH₃CN (28 mg, 0.44 mmol) in portions at 0° C. under a nitrogen atmosphere. The mixture was stirred for 16 hours at room temperature. The mixture was diluted with water (5 mL), and the aqueous phase was extracted with ethyl acetate (10 mL*3). The combined organic layers were washed with brine, dried over sodium sulfate, filtered and concentrated in vacuo. The residue was purified by re verse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl) (4-fluorobicyclo[2.2.1]heptan-1-yl)me thyl)-4-((4-methoxybenzyl)amino)cyclopentane-1-carboxamide (50 mg, 84 μmol) as a yellow oil. LCMS RT 0.847 min, [M+H]⁺ 594, LCMS method C.

### Step 2. Synthesis of (3S,4R)-3-acetamido-N—((S)-(2,3-dichloro-6-fluorophenyl)(4-fluorobicyclo[2.2.1]heptan-1-yl)methyl)-4-((4-methoxybenzyl)(methyl)amino)cyclopentane-1-carboxamide

To a mixture of (3S,4R)-3-acetamido-N—((S)-(2,3-dichloro-6-fluorophenyl)(4-fluorobicyclo[2.2.1]heptan-1-yl)methyl)-4-((4-methoxybenzyl)amino)cyclopentane-1-carboxamid e (50 mg, 84 μmol) and paraformaldehyde (3.8 mg, 0.13 mmol) in MeOH (4 mL) was added NaBH₃CN (16 mg, 0.25 mmol) in portions at 0° C. under a nitrogen atmosphere. The mixture was stirred for 16 hours at room temperature. The mixture was diluted with water (5 mL), and the aqueous phase was extracted with ethyl acetate (10 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl)(4-fluorobicyclo[2.2.1]heptan-1-yl)methyl)-4-((4-methoxybenzyl)(methyl)amino)cyclopentane-1-carboxamide (40 mg, 66 μmol) as a yellow oil. LCMS RT 0.896 min, [M+H]⁺ 608, LCMS method C.

### Step 3. Synthesis of (3S,4R)-3-acetamido-N—((S)-(2,3-dichloro-6-fluorophenyl) (4-fluorobicyclo[2.2.1]heptan-1-yl)methyl)-4-(methylamino)cyclopentane-1-carboxamide

To a mixture of (3S,4R)-3-acetamido-N—((S)-(2,3-dichloro-6-fluorophenyl)(4-fluorobicyclo[2.2.1]heptan-1-yl)methyl)-4-((4-methoxybenzyl)(methyl)amino)cyclopentane-1-carboxamide (40 mg, 66 μmol) in acetonitrile/H₂O (2.2 ml, 10:1) was added ceric ammonium nitrate (0.36 g, 0.66 mmol). The mixture was stirred at 20° C. for 3 hours. The reaction was quenched with water and extracted with ethyl acetate. The organic layer was washed with brine, dried over Na₂SO₄ and evaporated. The residue was purified by reverse phase flash chromatography (column: C18 silica gel; mobile phase A: water, mobile phase B: acetonitrile; gradient: 10% to 50% B in 10 min; detector: UV 254 nm) to give (3S,4R)-3-acetamido-N—((S)-(2,3-dichloro-6-fluorophenyl)(4-fluorobicyclo[2.2.1]heptan-1-yl)methyl)-4-(methylamino)cy-clopentane-1-carboxamide (24 mg, 49 μmol) as a yellow oil. LCMS RT 0.755 min, [M+H]⁺ 488, LCMS method C.

### Step 4. Synthesis of (1R,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide

(3S,4R)-3-acetamido-N—((S)-(2,3-dichloro-6-fluorophenyl)(4-fluorobicyclo[2.2.1]heptan-1-yl)methyl)-4-(methylamino)cyclopentane-1-carboxamide (24 mg, 49 μmol) was purified by chiral preparative HPLC (column: CHIRALPAK IE, 2*25 cm, 5 μm; mobile phase A: hexane (0.5% 2 M NH₃ in MeOH), mobile phase B: EtOH:DCM 1:1; flow rate: 20 mL/m in; gradient: 30% B isocratic; wavelength: 220/254 nm; RT1 (min): 8.86; RT2 (min): 10.32; sample solvent: EtOH:DCM 1:1; injection volume: 0.5 mL) to give (1R,3S,4R)-3-acetami-do-N—((S)-(2,3-dichloro-6-fluorophenyl)(4-fluorobicyclo[2.2.1]heptan-1-yl)methyl)-4-(meth ylamino)cyclopentane-1-carboxamide and (1S,3S,4R)-3-acetamido-N—((S)-(2,3-dichloro-6-fluorophenyl)(4-fluorobicyclo[2.2.1]heptan-1-yl)methyl)-4-(methylamino)cyclopentane-1-ca rboxamide, both as a white amorphous solid.

Isomer 1: 2 mg, 4 μmol. LCMS RT 1.772 min). ¹HNMR (400 MHz, DMSO-d₆) δ 8.07 (d, J=8.4 Hz, 1H), 7.82 (d, J=7.2 Hz, 1H), 7.62 (dd, J=9.0, 5.1 Hz, 1H), 7.26 (t, J=9.8 Hz, 1H), 5.52 (d, J=8.1 Hz, 1H), 3.92 (td, J=7.4, 3.6 Hz, 1H), 3.57 (p, J=6.0 Hz, 1H), 3.21 (d, J=13.8 Hz, 3H), 2.90 (p, J=8.4 Hz, 1H), 2.25-2.05 (m, 1H), 1.92 (dt, J=13.6, 8.2 Hz, 1H), 1.78 (d, J=3.5 Hz, 5H), 1.73 (s, 4H), 1.71 (dd, J=12.4, 8.7 Hz, 1H), 1.70-1.55 (m, 3H), 1.50 (s, 1H).

Isomer 2: 2.9 mg, 5.9 μmol. ¹H NMR (400 MHz, DMSO-d₆) δ 8.07 (d, J=8.3 Hz, 1H), 7.82 (d, J=7.3 Hz, 1H), 7.62 (dd, J=9.0, 5.1 Hz, 1H), 7.26 (t, J=9.8 Hz, 1H), 5.52 (d, J=8.2 Hz, 1H), 3.92 (tq, J=10.6, 5.4 Hz, 1H), 3.57 (p, J=6.1 Hz, 1H), 3.19 (s, 2H), 2.90 (p, J=8.4 Hz, 1H), 2.15 (ddt, J=28.7, 14.6, 7.6 Hz, 1H), 1.98-1.82 (m, 2H), 1.78 (d, J=3.5 Hz, 5H), 1.72 (s, 2H), 1.70-1.56 (m, 5H), 1.58-1.40 (m, 1H).

### Example 30

### (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide

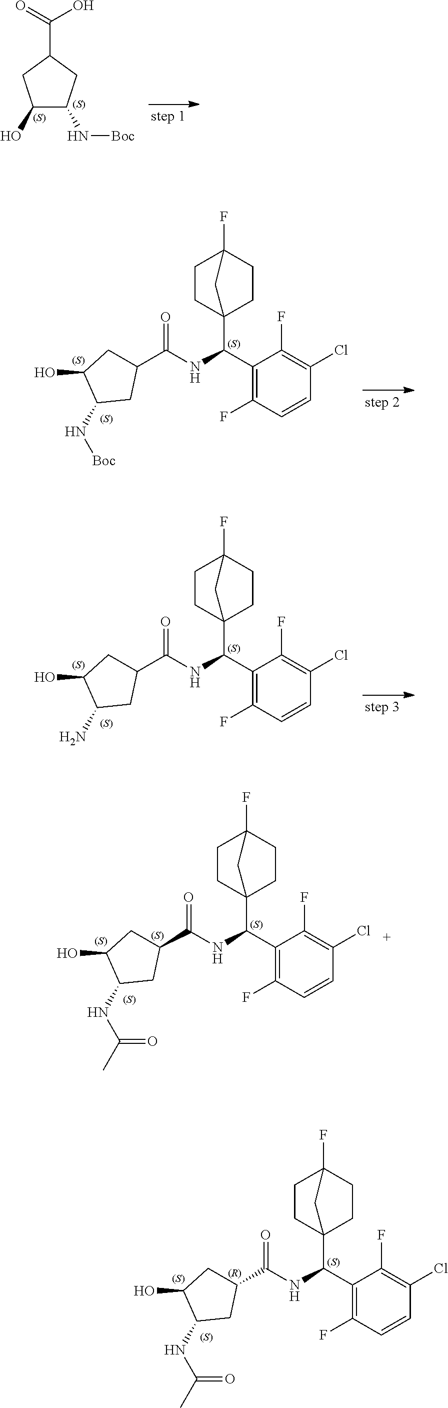

### Step 1. Synthesis of tert-butyl ((1S,2S)-4-(((S)-(3-chloro-2,6-difluorophenyl) (4-fluorobicyclo[2.2.1]heptan-1-yl)methyl) carbamoyl)-2-hydroxycyclopentyl)

(3S,4S)-3-((tert-butoxycarbonyl)amino)-4-hydroxycyclopentane-1-carboxylic acid was prepared using the same procedure in Example 31, from ethyl (3S,4R)-3-((tert-butoxycarbonyl)amino)-4-hydroxycyclopentane-1-carboxylate. To a mixture of (3S,4S)-3-((tert-butoxycarbonyl)amino)-4-hydroxycyclopentane-1-carboxylic acid (0.98 g, 4.0 mmol), (S)-(3-chloro-2,6-difluorophenyl)(4-fluorobicyclo[2.2.1]heptan-1-yl)methanamine (1.16 g, 4 mmol), NaHCO₃ (0.84 g, 0.01 mol) in DMF (10 mL) was added HATU (2.28 g, 6 mmol). The mixture was stirred for 1 h at 25° C. The reaction mixture was diluted with water (50 mL). The aqueous phase was extracted with ethyl acetate (50 ml) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 10 min; detector: UV 220 nm) to give tert-butyl ((1S,2S)-4-(((S)-(3-chloro-2,6-difluorophenyl)(4-fluorobicyclo[2.2.1]heptan-1-yl)methyl)carbamoyl)-2-hydroxycyclopentyl)carbamate (1.03 g, 2 mmol) as an off-white amorphous solid. LCMS RT 0.972 min, [M+H]⁺ 517.40, LCMS method C.

### Step 2. Synthesis of (3S,4S)-3-amino-N—((S)-(3-chloro-2,6-difluorophenyl) (4-fluorobicyclo[2.2.1]heptan-1-yl)methyl)-4-hydroxycyclopentane-1-carboxamide

A mixture of tert-butyl ((1S,2S)-4-(((S)-(3-chloro-2,6-difluorophenyl)(4-fluorobicyclo[2.2.1]heptan-1-yl)methyl)carbamoyl)-2-hydroxycyclopentyl)carbamate (500 mg, 967 μmol) in HCl (5 mL, 4 N in MeOH) was stirred for 30 min at 25° C. Concentration in vacuo gave (3S,4S)-3-amino-N—((S)-(3-chloro-2,6-difluorophenyl)(4-fluorobicyclo[2.2.1]heptan-1-yl)methyl)-4-hydroxycyclopentane-1-carboxamide (400 mg, 960 μmol) as a white solid. LCMS RT 0.918 min, [M+H]⁺ 417.15, LCMS method B.

### Step 3. Synthesis of (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide

To mixture of (3S,4S)-3-amino-N—((S)-(3-chloro-2,6-difluorophenyl)(4-fluorobicyclo[2.2.1]heptan-1-yl)methyl)-4-hydroxycyclopentane-1-carboxamide (390 mg, 936 μmol), acetic acid (169 mg, 2.81 mmol), TEA (283 mg, 2.81 mmol) in DMF (1 mL) was added T₃P (446 mg, 1.40 mmol). The mixture was stirred for 1 h at 25° C. The reaction mixture was diluted with water (10 mL), and the aqueous phase was extracted with ethyl acetate (20 ml) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 10 min; detector: UV 220 nm) to give an amorphous off-white solid. LCMS RT 0.721 min, [M+H]⁺ 517, LCMS method C.

The product was further purified by chiral preparative HPLC (column: CHIRALPAK ID, 2*25 cm, 5 μm; mobile phase A: hexane (0.5% 2 M NH₃ in MeOH), mobile phase B: EtOH:DCM 1:1; flow rate: 20 mL/min; gradient: 15% B isocratic; wavelength: 220/254 nm; RT1 (min): 7.41; RT2 (min): 9.34; sample solvent: EtOH:DCM 1:1; injection volume: 0.6 mL) to give (1S,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide and (1R,3S,4S)-3-acetamido-N—((S)-(3-chloro-2,6-difluorophenyl)(4-fluorobicyclo[2.2.1]heptan-1-yl)methyl)-4-hydroxycyclopentane-1-carboxamide, both as an off-white amorphous solid.

Isomer 1: 27.0 mg, 58.6 μmol. ¹H NMR (400 MHz, DMSO-d6) δ 8.24 (d, J=8.3 Hz, 1H), 7.79 (d, J=6.9 Hz, 1H), 7.57 (td, J=8.6, 5.4 Hz, 1H), 7.20-7.11 (m, 1H), 5.26 (d, J=8.3 Hz, 1H), 5.13 (d, J=4.6 Hz, 1H), 3.89-3.70 (m, 2H), 2.88 (p, J=8.1 Hz, 1H), 2.12-2.01 (m, 1H), 1.88 (dt, J=14.4, 7.6 Hz, 1H), 1.78 (s, 6H), 1.71 (d, J=13.5 Hz, 4H), 1.56 (ddd, J=13.7, 11.1, 6.7 Hz, 3H), 1.44 (q, J=7.4, 5.2 Hz, 2H). LCMS RT 0.878 min, [M+H]⁺ 459.35, LCMS method D.

Isomer 2: 15.5 mg, 33.4 μmol. ¹H NMR (400 MHz, DMSO-d6) δ 8.23 (d, J=8.3 Hz, 1H), 7.79 (d, J=6.7 Hz, 1H), 7.57 (td, J=8.7, 5.4 Hz, 1H), 7.16 (t, J=9.4 Hz, 1H), 5.26 (d, J=8.2 Hz, 1H), 5.10 (d, J=4.7 Hz, 1H), 3.79 (dp, J=19.7, 5.9 Hz, 2H), 2.88 (p, J=8.3 Hz, 1H), 2.06-1.95 (m, 2H), 1.79 (s, 7H), 1.71 (d, J=9.3 Hz, 4H), 1.63-1.54 (m, 2H), 1.45 (p, J=6.6 Hz, 3H). LCMS RT 0.888 min, [M+H]⁺ 459.35, LCMS method D.

### Example 31

### (1S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide

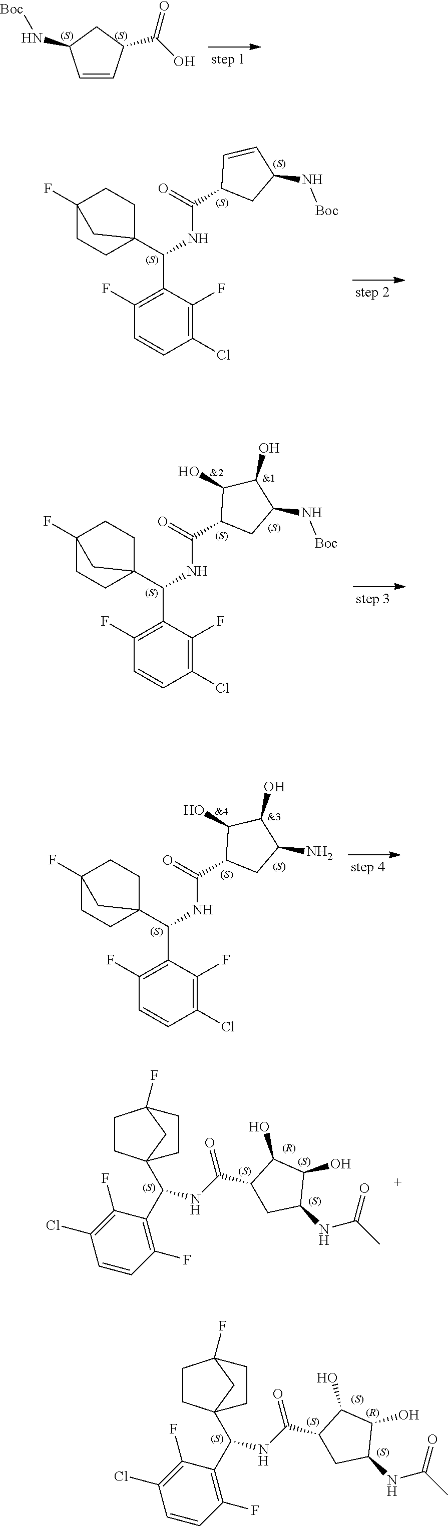

### Step 1. Synthesis of tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate

To a solution of (1S,4S)-4-((tert-butoxycarbonyl)amino)cyclopent-2-ene-1-carboxylic acid (150 mg, 660 μmol), (S)-(3-chloro-2,6-difluorophenyl)(4-fluorobicyclo[2.2.1]heptan-1-yl)methanamine (191 mg, 660 μmol) and NaHCO₃ (277 mg, 3.30 mmol) in DMF (2 mL) was added HATU (318 mg, 1.32 mmol). The mixture was stirred for 1 hour at 25° C. The reaction mixture was diluted with water (10 mL), and the aqueous phase was extracted with ethyl acetate (15 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 56% B to 79% B in 8 min, then 79% B; wavelength: 254 nm; RT1 (min): 7.63; injection volume: 0.8 mL). Lyophilization yielded tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate (190 mg, 381 μmol) as an off-white amorphous solid. LCMS RT 1.217 min, [M+H]⁺ 499.10, LCMS method B.

### Step 2. Synthesis of tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate

A mixture of tert-butyl ((1S,4S)-4-(((S)-(3-chloro-2,6-difluorophenyl)(4-fluorobicyclo[2.2.1]heptan-1-yl)methyl)carbamoyl)cyclopent-2-en-1-yl)carbamate (180 mg, 361 μmol), NMO (10.8 mg, 361 μmol), K₂OsO₄·2H₂O (11.1 mg, 36.1 μmol) in DCM (2 mL) was stirred for 1 hour at 25° C. The reaction mixture was diluted with water (10 mL), and the aqueous phase was extracted with ethyl acetate (15 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 56% B to 79% B in 8 min, then 79% B; wavelength: 254 nm; RT (min): 7.63; injection volume: 0.8 mL) to give tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate (140 mg, 263 μmol) as an off-white amorphous solid. LCMS RT 1.105 min, [M+H]⁺ 533.10, LCMS method C.

### Step 3. Synthesis of (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide

A mixture of tert-butyl ((1S,2RS,3SR,4S)-4-(((S)-(3-chloro-2,6-difluorophenyl)(4-fluorobicyclo[2.2.1]heptan-1-yl)methyl)carbamoyl)-2,3-dihydroxycyclopentyl)carbamate (130 mg, 244 μmol) in HCl (3 mL, 4 N in MeOH) was stirred for 2 hours at 25° C. The mixture was concentrated in vacuo to give (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide (150 mg) as a white amorphous solid. LCMS RT 0.913 min, [M+H]⁺ 433.30, LCMS method C.

### Step 4. Synthesis of (1S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide

To a solution of (1S,2RS,3SR,4S)-4-amino-N—((S)-(3-chloro-2,6-difluorophenyl)(4-fluorobicyclo[2.2.1]heptan-1-yl)methyl)-2,3-dihydroxycyclopentane-1-carboxamide (140 mg, 323 μmol), acetic acid (25.2 mg, 420 μmol) and NaHCO₃ (136 mg, 1.62 mmol) in DMF (2 mL) was added HATU (160 mg, 420 μmol). The mixture was stirred for 12 hours at 25° C. The reaction mixture was diluted with water (10 mL), and the aqueous phase was extracted with ethyl acetate (20 mL*3). The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 80% B in 25 min; detector: UV 254 nm) to give a white amorphous solid. LCMS RT 0.681 min, [M+H]⁺ 475.15, LCMS method B.

The material was further purified by preparative chiral HPLC (Column: CHIRALPAK IH3; mobile phase A: hexane (0.2% diethylamine), mobile phase B: EtOH:DCM 1:1); gradient: A:B 80:20 isocratic; flow rate: 1 mL/min; injection volume: 3 mL) to give (1S,2R,3S,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide and (1S,2S,3R,4S)-4-acetamido-N—((S)-(3-chloro-2,6-difluorophenyl)(4-fluorobicyclo[2.2.1]heptan-1-yl)methyl)-2,3-dihydroxycyclopentane-1-carboxamide, both as a white amorphous solid.

Isomer 1: 23.7 mg, 49.9 μmol. LCMS RT 0.950 min, [M+H]⁺ 475.10, LCMS method B. ¹HNMR (400 MHz, DMSO-d6) δ 8.16 (d, J=8.5 Hz, 1H), 7.87 (d, J=7.7 Hz, 1H), 7.69-7.48 (m, 1H), 7.16 (t, J=9.4 Hz, 1H), 5.31 (d, J=8.4 Hz, 1H), 4.93-4.52 (m, 2H), 3.86 (dd, J=7.9, 4.2 Hz, 2H), 3.56 (d, J=4.9 Hz, 1H), 2.94-2.63 (m, 1H), 2.05 (dt, J=13.2, 8.6 Hz, 1H), 1.91-1.53 (m, 11H), 1.44 (d, J=10.8 Hz, 2H), 1.27-1.07 (m, 1H).

Isomer 2: 2.8 mg, 5.9 μmol. LCMS RT 0.806 mm, [M+H]⁺ 475.00, LCMS method C. ¹H NMR (400 MHz, DMSO-d6) δ 8.53 (d, J=8.4 Hz, 1H), 7.58 (t, J=5.8 Hz, 2H), 7.16 (t, J=9.3 Hz, 1H), 5.32 (d, J=8.3 Hz, 1H), 5.13 (d, J=7.9 Hz, 1H), 4.97 (d, J=5.3 Hz, 1H), 4.14 (d, J=8.6 Hz, 1H), 4.04-3.89 (m, 1H), 3.67-3.59 (m, 1H), 2.96 (q, J=8.4 Hz, 1H), 1.75 (d, J=31.7 Hz, 13H), 1.51-1.34 (m, 1H).

### Example 32

### (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide, (1R,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide, (1S,4R)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide and (1R,4R)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide

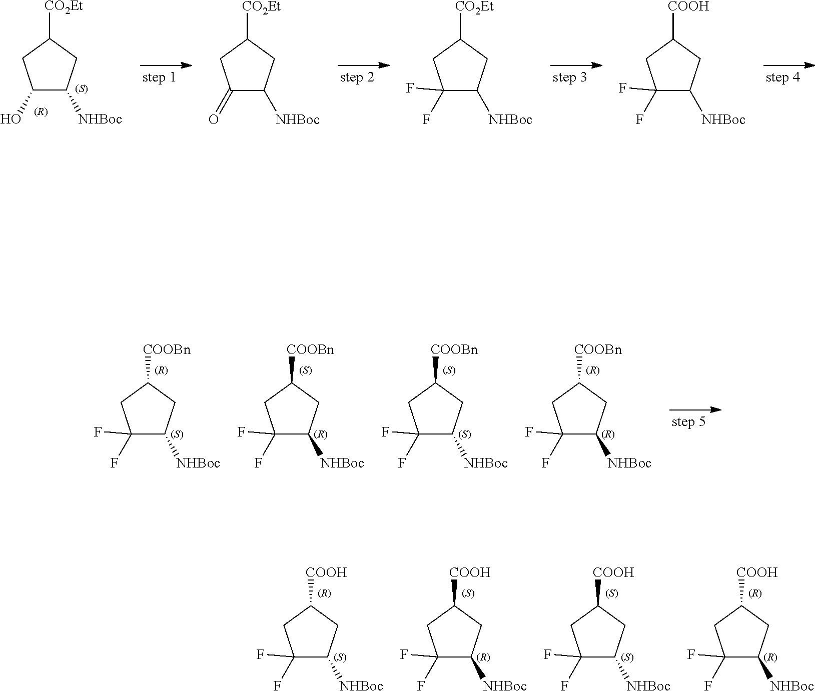

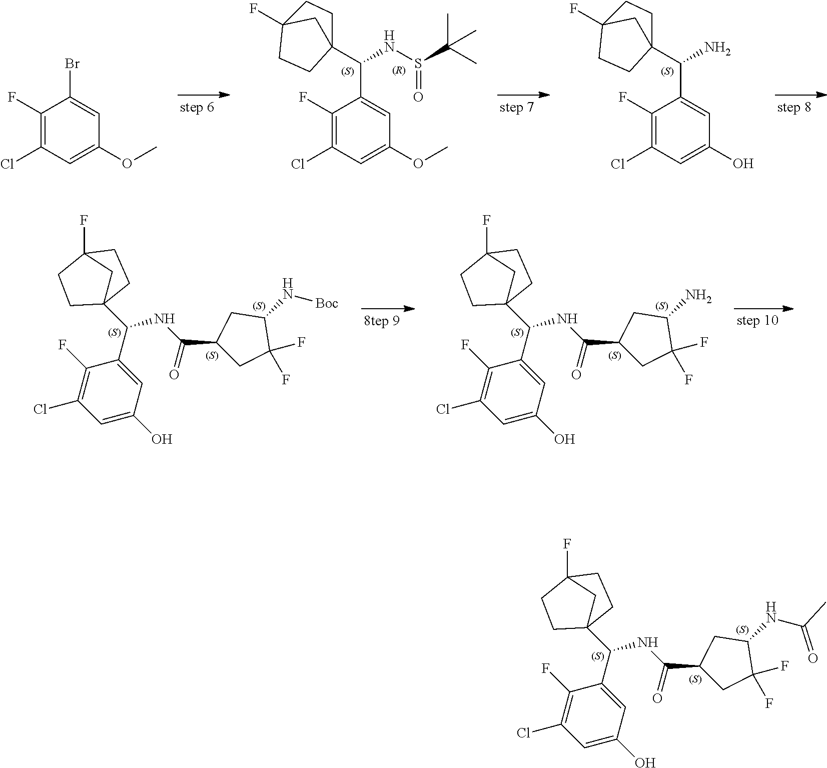

### Step 1. Synthesis of ethyl 3-((tert-butoxycarbonyl)amino)-4-oxocyclopentane-1-carboxylate

To a mixture of ethyl (3S,4R)-3-((tert-butoxycarbonyl)amino)-4-hydroxycyclopentane-1-carboxylate (130 g, 475 mmol) and 4 Å molecular sieves (40.0 g) in DCM (1.30 L) was added PCC (133 g, 618 mmol) at 25° C. The mixture was stirred at 25° C. for 1 hour. The mixture was diluted with MTBE (4.50 L) and filtered through celite under reduced pressure. The filtrate was concentrated under reduced pressure. The residue was purified by column chromatography (SiO₂, petroleum ether:ethyl acetate 20:1 to 0:1) to give ethyl 3-((tert-butoxycarbonyl)amino)-4-oxocyclopentane-1-carboxylate (71.1 g, 262 mmol) as a yellow oil. ¹H NMR: (400 MHz, DMSO-d₆) (7.09 (dd, J=8.0, 20.0 Hz, 1H), 4.10 (q, J=6.8 Hz, 2H), 3.99-3.77 (m, 1H), 3.26-3.01 (m, 1H), 2.48-2.40 (m, 1H), 2.39-2.20 (m, 2H), 2.12-1.81 (m, 1H), 1.37 (s, 9H), 1.19 (t, J=7.2 Hz, 3H).

### Step 2. Synthesis of ethyl 4-((tert-butoxycarbonyl)amino)-3,3-difluorocyclopentane-1-carboxylate

To a mixture of ethyl 3-((tert-butoxycarbonyl)amino)-4-oxocyclopentane-1-carboxylate (31.7 g, 117 mmol) in DCM (317 mL) was added DAST (77.3 mL, 585 mmol) at 0° C. under N₂. The mixture was warmed to 25° C. and stirred at 25° C. for 2 hours. The mixture was cooled to 0° C. and quenched with MeOH (150 mL). The mixture was stirred at 25° C. for 12 hours and concentrated under reduced pressure. The residue was purified by column chromatography (SiO₂, petroleum ether:ethyl acetate 50:1 to 3:1) to give ethyl 4-((tert-butoxycarbonyl)amino)-3,3-difluorocyclopentane-1-carboxylate (27.5 g, 93.8 mmol) as a brown oil. ¹H NMR: (400 MHz, DMSO-d₆) δ 7.19 (dd, J=9.2 Hz, 12.8 Hz, 1H), 4.20-3.98 (m, 3H), 3.13-2.95 (m, 1H), 2.45-2.08 (m, 3H), 1.95-1.69 (m, 1H), 1.39 (s, 9H), 1.21-1.16 (m, 3H).

### Step 3. Synthesis of 4-((tert-butoxycarbonyl)amino)-3,3-difluorocyclopentane-1-carboxylic acid

To a mixture of ethyl 4-((tert-butoxycarbonyl)amino)-3,3-difluorocyclopentane-1-carboxylate (28.5 g, 97.2 mmol) in MeOH (427 mL) and H₂O (140 mL) was added LiOH·H₂O (20.4 g, 486 mmol) at 0° C. The mixture was warmed to 25° C. and stirred at 25° C. for 2 hours. The mixture was concentrated under reduced pressure to remove most of MeOH. The residue was diluted with H₂O (300 mL). The mixture's pH was adjusted to 4 with saturated citric acid aqueous solution and extracted with DCM (300 mL*3). The combined organic layer was washed with brine (100 mL), dried over Na₂SO₄, filtered and concentrated under reduced pressure to obtain 4-((tert-butoxycarbonyl)amino)-3,3-difluorocyclopentane-1-carboxylic acid (25.7 g, crude) as a brown oil.

### Step 4. Synthesis of benzyl (1R,4S)-4-((tert-butoxycarbonyl)amino)-3,3-difluorocyclopentane-1-carboxylate, benzyl (1S,4S)-4-((tert-butoxycarbonyl)amino)-3,3-difluorocyclopentane-1-carboxylate, benzyl (1S,4R)-4-((tert-butoxycarbonyl)amino)-3,3-difluorocyclopentane-1-carboxylate and benzyl (1R,4R)-4-((tert-butoxycarbonyl)amino)-3,3-difluorocyclopentane-1-carboxylate

To a mixture of 4-((tert-butoxycarbonyl)amino)-3,3-difluorocyclopentane-1-carboxylic acid (25.7 g, 96.9 mmol) in DMF (260 mL) was added K₂CO₃ (26.8 g, 194 mmol) and BnBr (19.9 g, 116 mmol) at 25° C. The mixture was stirred at 25° C. for 2 hours. The mixture was poured into H₂O (1.00 L) under stirring. The mixture was extracted with ethyl acetate (500 mL*3), then combined organic phase was dried with Na₂SO₄, filtered and concentrated under reduced pressure. The residue was purified by column chromatography (SiO₂, petroleum ether:ethyl acetate 10:1 to 0:1) to give the product (34.0 g, crude) as a white solid.

The product (33.0 g) was purified by reverse phase HPLC (mobile phase A: 0.1% NH₄OH in water, mobile phase B: acetonitrile). The collected fractions were concentrated under reduced pressure to remove acetonitrile. The remaining aqueous solution was extracted with ethyl acetate (500 mL*3). The combined organic phase was dried over Na₂SO₄, filtered and concentrated under reduced pressure to give a solid (30.0 g). The residue was purified by chiral SFC (column: Daicel Chiralcel OJ 250 mm*50 mm, 10 μm; mobile phase: 12% isopropanol in hexane) to obtain peak 1 and peak 2.

Peak 1, after concentration, was further purified by chiral SFC (column: Daicel Chiralpak IG 250 mm*50 mm, 10 μm; mobile phase: 20% MeOH in 0.1% NH₄OH]) to obtain peak 3 and peak 4.

Peak 3 was concentrated under reduced pressure to give a white solid (5.20 g, 14.6 mmol). ¹⁹F NMR (376 MHz, DMSO-d₆) δ −101.23 ppm, −101.83 ppm, −107.06 ppm, −107.66 ppm. ¹H NMR: (400 MHz, DMSO-d₆) δ 7.41-7.31 (m, 5H), 7.22 (d, J=7.6 Hz, 1H), 5.12 (s, 2H), 4.26-4.05 (m, 1H), 3.27-3.09 (m, 1H), 2.46-2.32 (m, 2H), 2.26-2.12 (m, 1H), 1.98-1.83 (m, 1H), 1.39 (s, 9H).

Peak 4 was concentrated under reduced pressure to give a yellow solid (9.00 g, 25.3 mmol). ¹⁹F NMR: (376 MHz, DMSO-d₆) δ −101.23 ppm, −101.83 ppm, −107.07 ppm, −107.67 ppm. ¹H NMR: (400 MHz, DMSO-d₆) δ 7.41-7.31 (m, 5H), 7.22 (d, J=7.2 Hz, 1H), 5.12 (s, 2H), 4.26-4.05 (m, 1H), 3.27-3.09 (m, 1H), 2.46-2.32 (m, 2H), 2.26-2.12 (m, 1H), 1.98-1.83 (m, 1H), 1.39 (s, 9H).

Peak 2, after concentration, was further purified by chiral SFC (column: Daicel Chiralpak IG (250 mm*50 mm, 10 μm); mobile phase: 15% MeOH in 0.1% NH₄OH) to obtain peak 5 and peak 6.

Peak 5 was concentrated under reduced pressure to give a white solid (4.30 g, 12.1 mmol). ¹⁹F NMR: (376 MHz, DMSO-d₆) δ −100.03 ppm, −100.62 ppm, −103.25 ppm, −103.85 ppm. ¹H NMR (400 MHz, DMSO-d₆) δ 7.43-7.30 (m, 5H), 7.19 (d, J=8.8 Hz, 1H), 5.12 (s, 2H), 4.24-4.06 (m, 1H), 3.11 (br s, 1H), 2.46-2.16 (m, 3H), 1.85-7.30 (m, 1H), 1.38 (s, 9H).

Peak 6 was concentrated under reduced pressure to give a yellow solid (8.90 g, 25.0 mmol). ¹⁹F NMR: (376 MHz, DMSO-d₆) δ −100.03 ppm, −100.62 ppm, −103.25 ppm, −103.85 ppm

¹H NMR: (400 MHz, DMSO-d₆) δ 7.43-7.30 (m, 5H), 7.19 (d, J=9.2 Hz, 1H), 5.12 (s, 2H), 4.25-4.02 (m, 1H), 3.10-3.00 (m, 1H), 2.48-2.17 (m, 3H), 1.88-1.74 (m, 1H), 1.38 (s, 9H).

### Step 5. Synthesis of (1R,4S)-4-((tert-butoxycarbonyl)amino)-3,3-difluorocyclopentane-1-carboxylic acid, (1S,4S)-4-((tert-butoxycarbonyl)amino)-3,3-difluorocyclopentane-1-carboxylic acid, (1S,4R)-4-((tert-butoxycarbonyl)amino)-3,3-difluorocyclopentane-1-carboxylic acid and (1R,4R)-4-((tert-butoxycarbonyl)amino)-3,3-difluorocyclopentane-1-carboxylic acid

To a solution of peak 4 in step 4 (9.00 g, 25.3 mmol) in MeOH (135 mL) was added Pd/C (1.80 g, 10%) at 25° C. under N₂. The mixture was degassed and purged with H₂ 3 times. The mixture was stirred at 25° C. for 4 hours under H₂ (50 psi). The mixture was filtered through celite under reduced pressure. The filtrate was concentrated under reduced pressure to give a white solid (6.36 g, 25.6 mmol) as a white solid. ¹⁹F NMR: (376 MHz, DMSO-d₆) δ −101.09 ppm, −101.69 ppm, −106.70 ppm, −107.30 ppm. ¹H NMR: (400 MHz, DMSO-d₆) δ 12.58 (s, 1H), 7.19 (d, J=8.8 Hz, 1H), 4.30-3.96 (m, 1H), 3.08-2.88 (m, 1H), 2.38-2.28 (m, 2H), 2.17-2.11 (m, 1H), 1.90-1.80 (m, 1H), 1.39 (s, 9H).

The other 3 isomers were synthesized similarly.

### Step 6. Synthesis of (R)—N—((S)-(3-chloro-2-fluoro-5-methoxyphenyl) (4-fluorobicyclo[2.2.1]heptan-1-yl)methyl)-2-methylpropane-2-sulfinamide

To a mixture of 1-bromo-3-chloro-2-fluoro-5-methoxybenzene (1.2 g, 5.0 mmol) in THF (12 mL) was added n-butyllithium (2.4 mL, 2.5 M in THF, 6.0 mmol) dropwise at −78° C. under a nitrogen atmosphere. The mixture was stirred for 1 h at −78° C. prior to the addition of (R)—N-((4-fluorobicyclo[2.2.1]heptan-1-yl)methylene)-2-methylpropane-2-sulfinamide (981 mg, 4.0 mmol) at −78° C. The mixture was stirred for 1 hour at −78° C. The reaction was quenched with saturated NH₄Cl (aq.) and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 0% to 100% B in 20 min; detector: UV 220 nm) to afford (R)—N—((S)-(3-chloro-2-fluoro-5-methoxyphenyl)(4-fluorobicyclo[2.2.1]heptan-1-yl)methyl)-2-methylprop-ane-2-sulfinamide (1.21 g, 2.98 mmol) as a colorless oil. LCMS RT 1.118 min, [M+H]⁺ 405.90, LCMS method B.

### Step 7. (S)-3-(amino(4-fluorobicyclo[2.2.1]heptan-1-yl)methyl)-5-chloro-4-fluorophenol

A mixture of (R)—N—((S)-(3-chloro-2-fluoro-5-methoxyphenyl)(4-fluorobicyclo[2.2.1]heptan-1-yl)methyl)-2-methylpropane-2-sulfinamide (1.2 g, 3.0 mmol) in HBr (5 ml, 33% in AcOH) was stirred at 100° C. for 4 hours. The mixture was concentrated. The resulting crude material was purified by reverse phase flash chromatography (column: C18 silica gel; mobile phase A: water, mobile phase B: acetonitrile; gradient: 0% to 100% B in 10 min; detector: UV 220 nm) to give (S)-3-(amino(4-fluorobicyclo[2.2.1]heptan-1-yl)methyl)-5-chloro-4-fluorophenol (700 mg, 2.43 mmol) as an off-white solid. LCMS RT 0.917 min, [M+H]⁺ 288.05, LCMS method D.

### Step 8. Synthesis of tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate

To a solution of (S)-3-(amino(4-fluorobicyclo[2.2.1]heptan-1-yl)methyl)-5-chloro-4-fluorophenol (100 mg, 348 μmol), (1S,4S)-4-((tert-butoxycarbonyl)amino)-3,3-difluorocyclopentane-1-carboxylic acid (111 mg, 417 μmol), TEA (145 μL, 1.04 mmol) in DMF (1 mL) was added T₃P (166 mg, 521 μmol) at room temperature. The resulting mixture was stirred for 2 hours at room temperature. The reaction mixture was diluted with water (15 mL), and the aqueous phase was extracted with DCM (20 mL) three times. The combined organic layers were washed with brine, dried over sodium sulfate, filtered and concentrated in vacuo. The resulting crude material was purified by reverse phase flash chromatography (acetonitrile/water) to give tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate (130 mg, 69.9%) as a colorless oil. LCMS RT 0.892 min, [M+H]⁺ 535.00. LCMS method C.

### Step 9. Synthesis of (1S,4S)-4-amino-N—((S)-(3-chloro-2-fluoro-5-hydroxyphenyl)(4-fluorobicyclo[2.2.1]heptan-1-yl)methyl)-3,3-difluorocyclopentane-1-carboxamide

A mixture of tert-butyl ((1S,4S)-4-(((S)-(3-chloro-2-fluoro-5-hydroxyphenyl)(4-fluorobicyclo[2.2.1]heptan-1-yl)methyl)carbamoyl)-2,2-difluorocyclopentyl)carbamate (125 mg, 234 μmol) in HCl (4 N in MeOH, 3 mL) was stirred at room temperature for 2 hours. The mixture was concentrated. The resulting crude material was purified by reverse phase flash chromatography (column: C18 silica gel; mobile phase A: water, mobile phase B: acetonitrile; gradient: 0% to 100% B in 10 min; detector: UV 220 nm) to give (1S,4S)-4-amino-N—((S)-(3-chloro-2-fluoro-5-hydroxyphenyl)(4-fluorobicyclo[2.2.1]heptan-1-yl)methyl)-3,3-difluor-ocyclopentane-1-carboxamide (100 mg, 230 μmol) as a colorless oil. LCMS RT 0.717 min, [M+H]⁺ 435.00, LCMS method C.

### Step 10. Synthesis of (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide

To a mixture of (1S,4S)-4-amino-N—((S)-(3-chloro-2-fluoro-5-hydroxyphenyl)(4-fluorobicyclo[2.2.1]heptan-1-yl)methyl)-3,3-difluorocyclopentane-1-carboxamide (50 mg, 0.11 mmol), NaHCO₃ (48 mg, 0.57 mmol) and acetic acid (8.3 mg, 0.14 mmol) in DMF (1 mL) was added HATU (66 mg, 0.17 mmol) at room temperature. The resulting mixture was stirred for 2 hours at room temperature. The reaction mixture was diluted with water (10 mL), a nd the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Pr ep OBD C18 Column, 30*150 mm, 5 μm; flow rate: 60 mL/min; gradient: 23% B to 50% B in 8 min, then 50% B; wavelength: 220 nm; RT1 (min): 7.58) to give (1S,4S)-4-acetamido-N—((S)-(3-chloro-2-fluoro-5-hydroxyphenyl)(4-fluorobicyclo[2.2.1]heptan-1-yl)methyl)-3,3-difluorocyclopentane-1-carboxamide (29.7 mg, 62.3 μmol) as an off-white amorphous solid.

¹H NMR (400 MHz, DMSO-d6) δ 9.81 (s, 1H), 8.37 (d, J=8.6 Hz, 1H), 8.08 (d, J=8.8 Hz, 1 H), 6.79 (dd, J=5.8, 2.9 Hz, 1H), 6.65 (dd, J=5.4, 2.9 Hz, 1H), 5.20 (d, J=8.7 Hz, 1H), 4.48 (dt, J=18.1, 9.2 Hz, 11H), 3.08 (s, 11H), 2.37-2.17 (m, 2H), 2.04-1.94 (m, 11H), 1.86 (s, 4H), 1.81-1.64 (m, 6H), 1.62-1.52 (m, 2H), 1.36 (s, 2H). LCMS RT 0.899 min [M−H]⁻ 475.15, LCMS method D.

Additional compounds prepared according to the methods of Examples 1-32 are listed in Table 2 below. Corresponding ¹H NMR and mass spectrometry characterization for these compounds are described in Table 1. Certain compounds in Table 2 below were prepared with other compounds whose preparation is described further below in the Examples.

TABLE 2
Additional exemplary compounds

| | | | | |
|---|---|---|---|---|
| I-83 | I-247 | I-248 | I-252 | I-253 |
| I-257 | I-258 | I-259 | I-260 | I-263 |
| I-266 | I-325 | I-327 | I-333 | I-334 |
| I-337 | I-338 | I-339 | I-340 | I-341 |
| I-342 | I-343 | I-344 | I-348 | I-349 |
| I-351 | I-352 | I-353 | I-354 | I-369 |
| I-370 | I-371 | I-372 | I-378 | I-384 |
| I-403 | I-406 | I-424 | I-427 | I-432 |
| I-445 | I-446 | I-454 | I-455 | I-460 |
| I-461 | I-466 | I-467 | I-469 | I-479 |
| I-494 | I-507 | I-510 | I-621 | I-630 |
| I-634 | I-635 | I-666 | I-670 | I-691 |
| I-692 | I-693 | I-694 | I-695 | I-696 |
| I-700 | I-701 | I-702 | I-703 | I-704 |
| I-709 | I-710 | I-711 | I-712 | I-717 |
| I-718 | I-719 | I-724 | I-725 | I-730 |
| I-794 | I-802 | I-803 | I-804 | I-806 |
| I-808 | I-817 | I-823 | I-825 | I-830 |
| I-838 | I-841 | I-842 | I-843 | I-844 |
| I-846 | I-847 | I-848 | I-849 | I-850 |
| I-851 | I-852 | I-858 | I-859 | I-860 |
| I-861 | I-862 | I-863 | I-872 | I-877 |
| I-878 | I-879 | I-882 | I-883 | I-887 |
| I-891 | I-897 | I-898 | I-900 | I-901 |
| I-902 | I-903 | I-904 | I-905 | I-906 |
| I-907 | I-908 | I-909 | I-910 | I-911 |
| I-912 | I-913 | I-914 | I-915 | I-916 |
| I-917 | I-918 | I-919 | I-925 | I-927 |
| I-929 | I-931 | I-932 | I-934 | I-935 |
| I-936 | I-937 | I-941 | I-942 | I-943 |
| I-944 | I-945 | I-946 | I-948 | I-949 |
| I-950 | I-951 | I-952 | I-953 | I-954 |
| I-955 | I-956 | I-957 | I-958 | I-959 |
| I-960 | I-961 | I-962 | I-963 | I-964 |
| I-965 | I-966 | I-967 | I-968 | I-980 |
| I-981 | I-982 | I-983 | I-984 | I-985 |
| I-986 | I-987 | I-988 | I-989 | I-992 |
| I-993 | I-996 | I-997 | I-998 | I-999 |
| I-1000 | I-1001 | I-1002 | I-1003 | I-1004 |
| I-1005 | I-1011 | I-1012 | I-1013 | I-1014 |
| I-1017 | I-1018 | I-1019 | I-1025 | I-1026 |
| I-1028 | I-1029 | I-1030 | I-1031 | I-1032 |
| I-1033 | I-1034 | I-1035 | I-1036 | I-1037 |
| I-1038 | I-1039 | I-1040 | I-1041 | I-1045 |
| I-1046 | I-1047 | I-1048 | I-1049 | I-1050 |
| I-1051 | I-1052 | I-1053 | I-1054 | I-1055 |
| I-1056 | I-1057 | I-1058 | I-1059 | I-1060 |
| I-1061 | I-1062 | I-1063 | I-1064 | I-1065 |
| I-1066 | I-1067 | I-1068 | I-1069 | I-1070 |
| I-1071 | I-1072 | I-1073 | I-1074 | I-1075 |
| I-1076 | I-1077 | I-1078 | I-1079 | I-1080 |
| I-1081 | I-1082 | I-1083 | I-1084 | I-1085 |
| I-1086 | I-1087 | I-1088 | I-1089 | I-1090 |
| I-1091 | I-1092 | I-1093 | I-1094 | I-1095 |
| I-1096 | I-1097 | I-1098 | I-1099 | I-1100 |
| I-1101 | I-1102 | I-1103 | I-1104 | I-1105 |
| I-1106 | I-1107 | I-1108 | I-1109 | I-1110 |
| I-1111 | I-1115 | I-1120 | I-1121 | I-1122 |
| I-1123 | I-1124 | I-1125 | I-1126 | I-1127 |
| I-1128 | I-1129 | I-1130 | I-1131 | I-1132 |
| I-1133 | I-1134 | I-1135 | I-1136 | I-1137 |
| I-1138 | I-1139 | I-1140 | I-1141 | I-1142 |
| I-1143 | I-1144 | I-1145 | I-1146 | I-1147 |
| I-1148 | I-1149 | I-1150 | I-1151 | I-1152 |
| I-1153 | I-1154 | I-1155 | I-1156 | I-1157 |
| I-1158 | I-1159 | I-1160 | I-1161 | I-1162 |
| I-1163 | I-1164 | I-1165 | I-1166 | I-1167 |
| I-1168 | I-1169 | I-1170 | I-1171 | I-1172 |
| I-1173 | I-1174 | I-1175 | I-1176 | I-1177 |
| I-1178 | I-1179 | I-1180 | I-1181 | I-1182 |
| I-1183 | I-1184 | I-1185 | I-1186 | I-1187 |
| I-1188 | I-1189 | I-1190 | I-1191 | I-1192 |
| I-1193 | I-1194 | I-1195 | I-1196 | I-1197 |
| I-1198 | I-1200 | I-1201 | I-1203 | I-1204 |
| I-1205 | I-1206 | I-1207 | I-1208 | I-1209 |
| I-1210 | I-1211 | I-1212 | I-1213 | I-1217 |
| I-1218 | I-1219 | I-1221 | I-1226 | I-1227 |
| I-1228 | I-1229 | I-1230 | I-1231 | I-1232 |
| I-1233 | I-1234 | I-1235 | I-1236 | I-1237 |
| I-1238 | I-1239 | I-1240 | I-1241 | I-1242 |
| I-1243 | I-1244 | I-1245 | I-1246 | I-1247 |
| I-1248 | I-1249 | I-1250 | I-1251 | I-1252 |
| I-1253 | I-1254 | I-1255 | I-1256 | I-1257 |
| I-1258 | I-1261 | I-1262 | I-1263 | I-1264 |
| I-1265 | I-1266 | I-1267 | I-1268 | I-1269 |
| I-1270 | I-1271 | I-1272 | I-1273 | I-1274 |
| I-1279 | I-1280 | I-1281 | I-1282 | I-1283 |
| I-1284 | I-1285 | I-1286 | I-1287 | I-1288 |
| I-1289 | I-1290 | I-1291 | I-1292 | I-1293 |
| I-1294 | I-1295 | I-1296 | I-1297 | I-1298 |
| I-1299 | I-1300 | I-1301 | I-1302 | I-1303 |
| I-1304 | I-1305 | I-1306 | I-1307 | I-1308 |
| I-1309 | I-1310 | I-1311 | I-1312 | I-1313 |
| I-1314 | I-1315 | I-1316 | I-1317 | I-1318 |
| I-1319 | I-1320 | I-1321 | I-1322 | I-1323 |
| I-1324 | I-1325 | I-1326 | I-1327 | I-1336 |
| I-1337 | I-1338 | I-1339 | I-1340 | I-1341 |
| I-1342 | I-1344 | I-1345 | I-1346 | I-1347 |
| I-1348 | I-1349 | I-1350 | I-1351 | I-1352 |
| I-1353 | I-1354 | I-1355 | I-1356 | I-1357 |
| I-1358 | I-1359 | I-1360 | I-1361 | I-1362 |
| I-1364 | I-1365 | I-1366 | I-1367 | I-1368 |
| I-1369 | I-1370 | I-1371 | I-1372 | I-1373 |
| I-1374 | I-1375 | I-1376 | I-1377 | I-1378 |
| I-1379 | I-1380 | I-1381 | I-1382 | I-1383 |
| I-1384 | I-1385 | I-1386 | I-1387 | I-1388 |
| I-1389 | I-1390 | I-1391 | I-1392 | I-1393 |
| I-1394 | I-1395 | I-1396 | I-1397 | I-1398 |
| I-1399 | I-1400 | I-1401 | I-1402 | I-1403 |
| I-1404 | I-1405 | I-1406 | I-1407 | I-1408 |
| I-1409 | I-1410 | I-1411 | I-1412 | I-1413 |
| I-1414 | I-1415 | I-1416 | I-1417 | I-1418 |
| I-1419 | I-1420 | I-1421 | I-1422 | I-1423 |
| I-1424 | I-1425 | I-1426 | I-1427 | I-1428 |
| I-1429 | I-1430 | I-1431 | I-1432 | I-1433 |
| I-1434 | I-1435 | I-1436 | I-1437 | I-1438 |
| I-1439 | I-1440 | I-1441 | I-1442 | I-1443 |
| I-1444 | I-1445 | I-1446 | I-1447 | I-1448 |
| I-1449 | I-1450 | I-1451 | I-1452 | I-1453 |
| I-1454 | I-1455 | I-1456 | I-1457 | I-1458 |
| I-1459 | I-1460 | I-1461 | I-1462 | I-1463 |
| I-1464 | I-1465 | I-1466 | I-1467 | I-1468 |
| I-1469 | I-1470 | I-1471 | I-1472 | I-1473 |
| I-1474 | I-1475 | I-1476 | I-1477 | I-1478 |
| I-1479 | I-1480 | I-1481 | I-1482 | I-1483 |
| I-1484 | I-1485 | I-1486 | I-1487 | I-1488 |
| I-1489 | I-1490 | I-1491 | I-1492 | I-1494 |
| I-1495 | I-1496 | I-1497 | I-1498 | I-1499 |
| I-1500 | I-1501 | I-1502 | I-1503 | I-1504 |
| I-1505 | I-1517 | I-1518 | I-1519 | I-1520 |
| I-1521 | I-1522 | I-1523 | I-1524 | I-1525 |
| I-1526 | I-1527 | I-1528 | I-1529 | I-1530 |
| I-1531 | I-1532 | I-1533 | I-1534 | I-1535 |
| I-1536 | I-1537 | I-1538 | I-1539 | I-1540 |
| I-1541 | I-1542 | I-1543 | I-1544 | I-1545 |
| I-1546 | I-1547 | I-1548 | I-1549 | I-1550 |
| I-1551 | I-1552 | I-1553 | I-1554 | I-1555 |
| I-1556 | I-1557 | I-1558 | I-1559 | I-1560 |
| I-1561 | I-1562 | I-1563 | I-1564 | I-1565 |
| I-1566 | I-1567 | I-1568 | I-1569 | I-1570 |
| I-1571 | I-1572 | I-1573 | I-1574 | I-1575 |
| I-1576 | I-1577 | I-1578 | I-1579 | I-1580 |
| I-1581 | I-1582 | I-1583 | I-1584 | I-1585 |
| I-1586 | I-1587 | I-1588 | I-1589 | I-1590 |
| I-1591 | I-1592 | I-1593 | I-1594 | I-1595 |
| I-1596 | I-1597 | I-1598 | I-1599 | I-1600 |
| I-1601 | I-1602 | I-1603 | I-1604 | I-1605 |
| I-1606 | I-1607 | I-1608 | I-1609 | I-1610 |
| I-1611 | I-1612 | I-1613 | I-1614 | I-1615 |
| I-1616 | I-1617 | I-1618 | I-1619 | I-1620 |
| I-1621 | I-1622 | I-1623 | I-1624 | I-1625 |
| I-1626 | I-1627 | I-1628 | I-1629 | I-1630 |
| I-1631 | I-1632 | I-1633 | I-1634 | I-1635 |
| I-1636 | I-1637 | I-1638 | I-1639 | I-1643 |
| I-1644 | I-1645 | I-1646 | I-1647 | I-1648 |
| I-1649 | I-1650 | I-1652 | I-1653 | I-1654 |
| I-1655 | I-1656 | I-1657 | I-1658 | I-1659 |
| I-1660 | I-1661 | I-1662 | I-1663 | I-1664 |
| I-1665 | I-1666 | I-1667 | I-1668 | I-1669 |
| I-1670 | I-1671 | I-1672 | I-1673 | I-1674 |
| I-1675 | I-1676 | I-1677 | I-1679 | I-1680 |
| I-1681 | I-1682 | I-1683 | I-1684 | I-1686 |
| I-1687 | I-1688 | I-1689 | I-1690 | I-1691 |
| I-1692 | I-1693 | I-1694 | I-1695 | I-1696 |
| I-1697 | I-1698 | I-1699 | I-1700 | I-1701 |
| I-1702 | I-1703 | I-1704 | I-1705 | I-1706 |
| I-1707 | I-1708 | I-1709 | I-1710 | I-1711 |
| I-1712 | I-1713 | I-1714 | I-1715 | I-1716 |
| I-1717 | I-1718 | I-1719 | I-1720 | I-1721 |
| I-1722 | I-1723 | I-1724 | I-1725 | I-1726 |
| I-1727 | I-1728 | I-1729 | I-1730 | I-1731 |
| I-1732 | I-1733 | I-1734 | I-1735 | I-1736 |
| I-1737 | I-1738 | I-1739 | I-1740 | I-1741 |
| I-1742 | I-1743 | I-1744 | I-1745 | I-1746 |
| I-1747 | I-1748 | I-1749 | I-1750 | I-1751 |
| I-1752 | I-1753 | I-1754 | I-1755 | I-1756 |
| I-1757 | I-1758 | I-1759 | I-1760 | I-1761 |
| I-1762 | I-1763 | I-1764 | I-1765 | I-1766 |
| I-1767 | I-1768 | I-1769 | I-1770 | I-1771 |
| I-1772 | I-1773 | I-1774 | I-1775 | I-1776 |
| I-1777 | I-1778 | I-1779 | I-1780 | I-1781 |
| I-1782 | I-1783 | I-1784 | I-1785 | I-1786 |
| I-1787 | I-1788 | I-1789 | I-1791 | I-1792 |
| I-1793 | I-1794 | I-1795 | I-1796 | I-1797 |
| I-1798 | I-1799 | I-1800 | I-1801 | I-1814 |
| I-1815 | I-1828 | I-1829 | I-1833 | I-1834 |
| I-1835 | I-1836 | I-1837 | I-1838 | I-1840 |
| I-1841 | I-1842 | I-1843 | I-1845 | I-1846 |
| I-1847 | I-1848 | I-1849 | I-1850 | I-1851 |
| I-1852 | I-1854 | I-1855 | I-1856 | I-1857 |
| I-1858 | I-1859 | I-1860 | I-1861 | I-1862 |
| I-1863 | I-1864 | I-1865 | I-1866 | I-1867 |
| I-1868 | I-1869 | I-1870 | I-1871 | I-1873 |
| I-1874 | I-1875 | I-1876 | I-1877 | I-1878 |
| I-1879 | I-1880 | I-1881 | I-1882 | I-1883 |
| I-1884 | I-1885 | I-1886 | I-1887 | I-1888 |
| I-1889 | I-1890 | I-1891 | I-1892 | I-1893 |
| I-1895 | I-1896 | I-1897 | I-1898 | I-1899 |
| I-1900 | I-1901 | I-1902 | I-1903 | I-1904 |
| I-1906 | I-1907 | I-1908 | I-1909 | I-1910 |
| I-1911 | I-1912 | I-1913 | I-1914 | I-1916 |
| I-1917 | I-1918 | I-1921 | I-1922 | I-1923 |
| I-1924 | I-1925 | I-1926 | I-1927 | I-1928 |
| I-1929 | I-1930 | I-1931 | I-1932 | I-1934 |
| I-1935 | I-1936 | I-1937 | I-1938 | I-1939 |
| I-1940 | I-1941 | I-1942 | I-1943 | I-1944 |
| I-1945 | I-1946 | I-1947 | I-1948 | I-1949 |
| I-1950 | I-1951 | I-1952 | I-1953 | I-1954 |
| I-1955 | I-1956 | I-1957 | I-1961 | I-1962 |
| I-1963 | I-1964 | I-1965 | I-1966 | I-1967 |
| I-1968 | I-1970 | I-1971 | I-1972 | I-1973 |
| I-1974 | I-1975 | I-1976 | I-1977 | I-1978 |
| I-1979 | I-1980 | I-1981 | I-1984 | I-1985 |
| I-1986 | I-1987 | I-1988 | I-1989 | I-1990 |
| I-1991 | I-1992 | I-1993 | I-1994 | I-1995 |
| I-1996 | I-1997 | I-1998 | I-1999 | I-2000 |
| I-2001 | I-2002 | I-2003 | I-2004 | I-2005 |
| I-2006 | I-2007 | I-2008 | I-2010 | I-2011 |
| I-2012 | I-2013 | I-2018 | I-2019 | I-2020 |
| I-2021 | I-2030 | I-2031 | I-2032 | I-2033 |
| I-2034 | I-2035 | I-2036 | I-2040 | I-2041 |
| I-2042 | I-2043 | I-2044 | I-2045 | I-2046 |
| I-2047 | I-2048 | I-2049 | I-2050 | I-2051 |
| I-2052 | I-2053 | I-2054 | I-2056 | I-2057 |
| I-2059 | I-2060 | I-2061 | I-2062 | I-2063 |
| I-2064 | I-2065 | I-2066 | I-2067 | I-2068 |
| I-2069 | I-2070 | I-2071 | I-2072 | I-2073 |
| I-2074 | I-2075 | I-2076 | I-2077 | I-2078 |
| I-2079 | I-2080 | I-2081 | I-2082 | I-2083 |
| I-2084 | I-2085 | I-2086 | I-2087 | I-2088 |
| I-2089 | I-2090 | I-2091 | I-2092 | I-2095 |
| I-2096 | I-2097 | I-2098 | I-2099 | I-2100 |
| I-2101 | I-2102 | I-2103 | I-2104 | I-2105 |
| I-2106 | I-2107 | I-2109 | I-2110 | I-2112 |
| I-2113 | I-2114 | I-2115 | I-2116 | I-2117 |
| I-2118 | I-2119 | I-2129 | I-2131 | I-2133 |
| I-2134 | I-2136 | I-2137 | I-2138 | I-2139 |
| I-2140 | I-2141 | I-2142 | I-2143 | I-2144 |
| I-2145 | I-2146 | I-2147 | I-2148 | I-2149 |
| I-2150 | I-2154 | I-2155 | I-2157 | I-2158 |
| I-2161 | I-2162 | I-2163 | I-2164 | I-2165 |
| I-2166 | I-2167 | I-2168 | I-2169 | I-2170 |
| I-2171 | I-2172 | I-2173 | I-2174 | I-2175 |
| I-2176 | I-2177 | I-2178 | I-2179 | I-2180 |
| I-2181 | I-2182 | I-2183 | I-2184 | I-2185 |
| I-2186 | I-2187 | I-2188 | I-2189 | I-2190 |
| I-2191 | I-2192 | I-2193 | I-2194 | I-2195 |
| I-2196 | I-2197 | I-2198 | I-2199 | I-2200 |
| I-2201 | I-2202 | I-2203 | I-2204 | I-2205 |
| I-2206 | I-2207 | I-2208 | I-2209 | I-2210 |
| I-2211 | I-2212 | I-2213 | I-2214 | I-2215 |
| I-2216 | I-2217 | I-2218 | I-2219 | I-2220 |
| I-2222 | I-2223 | I-2224 | I-2225 | I-2226 |
| I-2227 | I-2228 | I-2229 | I-2230 | I-2231 |
| I-2232 | I-2233 | I-2234 | I-2235 | I-2236 |
| I-2237 | I-2238 | I-2239 | I-2240 | I-2241 |
| I-2242 | I-2243 | I-2244 | I-2245 | I-2246 |
| I-2247 | I-2248 | I-2249 | I-2250 | I-2251 |
| I-2252 | I-2253 | I-2254 | I-2255 | I-2256 |
| I-2257 | I-2258 | I-2259 | I-2260 | I-2261 |
| I-2262 | I-2263 | I-2264 | I-2265 | I-2266 |
| I-2267 | I-2268 | I-2269 | I-2271 | I-2272 |
| I-2274 | I-2275 | I-2276 | I-2277 | I-2278 |
| I-2279 | I-2280 | I-2281 | I-2282 | I-2283 |
| I-2284 | I-2285 | I-2286 | I-2287 | I-2288 |
| I-2289 | I-2290 | I-2291 | I-2292 | I-2293 |
| I-2294 | I-2295 | I-2296 | I-2297 | I-2298 |
| I-2299 | I-2300 | I-2301 | I-2302 | I-2303 |
| I-2304 | I-2305 | I-2306 | I-2307 | I-2308 |
| I-2309 | I-2310 | I-2311 | I-2313 | I-2314 |
| I-2316 | I-2317 | I-2318 | I-2319 | I-2320 |
| I-2322 | I-2323 | I-2325 | I-2326 | I-2327 |
| I-2328 | I-2329 | I-2330 | I-2331 | I-2332 |
| I-2333 | I-2334 | I-2335 | I-2336 | I-2337 |
| I-2338 | I-2339 | I-2340 | I-2341 | I-2342 |
| I-2344 | I-2345 | I-2346 | I-2348 | I-2349 |
| I-2350 | I-2351 | I-2352 | I-2353 | I-2354 |
| I-2355 | I-2356 | I-2357 | I-2358 | I-2359 |
| I-2360 | I-2361 | I-2362 | I-2363 | I-2364 |
| I-2365 | I-2366 | I-2367 | I-2368 | I-2369 |
| I-2370 | I-2371 | I-2372 | I-2373 | I-2374 |
| I-2375 | I-2376 | I-2377 | I-2378 | I-2379 |
| I-2380 | I-2381 | I-2382 | I-2383 | I-2384 |
| I-2385 | I-2386 | I-2387 | I-2388 | I-2389 |
| I-2390 | I-2391 | I-2392 | I-2393 | I-2394 |
| I-2395 | I-2396 | I-2397 | I-2398 | I-2399 |
| I-2400 | I-2401 | I-2402 | I-2403 | I-2404 |
| I-2405 | I-2406 | I-2407 | I-2408 | I-2409 |
| I-2410 | I-2411 | I-2412 | I-2413 | I-2414 |
| I-2417 | I-2418 | I-2419 | I-2420 | I-2421 |
| I-2422 | I-2423 | I-2424 | I-2425 | I-2426 |
| I-2427 | I-2428 | I-2429 | I-2430 | I-2431 |
| I-2432 | I-2433 | I-2434 | I-2435 | I-2436 |
| I-2437 | I-2438 | I-2439 | I-2440 | I-2441 |
| I-2442 | I-2443 | I-2444 | I-2445 | I-2446 |
| I-2447 | I-2448 | I-2449 | I-2450 | I-2451 |
| I-2452 | I-2453 | I-2454 | I-2455 | I-2456 |
| I-2457 | I-2458 | I-2459 | I-2460 | I-2461 |
| I-2462 | I-2463 | I-2464 | I-2465 | I-2466 |
| I-2467 | I-2468 | I-2469 | I-2470 | I-2471 |
| I-2472 | I-2473 | I-2474 | I-2475 | I-2476 |
| I-2477 | I-2478 | I-2479 | I-2480 | I-2481 |
| I-2482 | I-2483 | I-2484 | I-2485 | I-2486 |
| I-2487 | I-2488 | I-2489 | I-2490 | I-2491 |
| I-2492 | I-2493 | I-2494 | I-2495 | I-2496 |
| I-2497 | I-2498 | I-2499 | I-2500 | I-2501 |
| I-2502 | I-2503 | I-2504 | I-2505 | I-2506 |
| I-2507 | I-2508 | I-2509 | I-2510 | I-2511 |
| I-2512 | I-2513 | I-2514 | I-2515 | I-2516 |
| I-2517 | I-2518 | I-2519 | I-2520 | I-2521 |
| I-2522 | I-2523 | I-2524 | I-2525 | I-2526 |
| I-2527 | I-2528 | I-2529 | I-2530 | I-2531 |
| I-2532 | I-2533 | I-2534 | I-2535 | I-2536 |
| I-2537 | I-2538 | I-2539 | I-2540 | I-2541 |
| I-2542 | I-2543 | I-2544 | I-2545 | I-2546 |
| I-2547 | I-2548 | I-2549 | I-2550 | I-2551 |
| I-2552 | I-2553 | I-2554 | I-2555 | I-2556 |
| I-2557 | I-2558 | I-2559 | I-2560 | I-2561 |
| I-2562 | I-2563 | I-2564 | I-2565 | I-2566 |
| I-2567 | I-2568 | I-2569 | I-2570 | I-2571 |
| I-2572 | I-2573 | I-2574 | I-2575 | I-2576 |
| I-2577 | I-2578 | I-2579 | I-2580 | I-2581 |
| I-2582 | I-2583 | I-2584 | I-2585 | I-2586 |
| I-2587 | I-2588 | I-2589 | I-2590 | I-2591 |
| I-2592 | I-2593 | I-2594 | I-2595 | I-2596 |
| I-2597 | I-2598 | I-2599 | I-2600 | I-2601 |
| I-2602 | I-2603 | I-2604 | I-2605 | I-2606 |
| I-2607 | I-2608 | I-2609 | I-2610 | I-2611 |
| I-2612 | I-2613 | I-2614 | I-2615 | I-2616 |
| I-2617 | I-2618 | I-2619 | I-2620 | I-2621 |
| I-2622 | I-2623 | I-2624 | I-2625 | I-2626 |
| I-2627 | I-2628 | I-2629 | I-2630 | I-2631 |
| I-2632 | I-2633 | I-2634 | I-2635 | I-2636 |
| I-2637 | I-2638 | I-2639 | I-2640 | I-2641 |
| I-2642 | I-2643 | I-2644 | I-2645 | I-2646 |
| I-2647 | I-2648 | I-2649 | I-2650 | I-2651 |
| I-2652 | I-2653 | I-2654 | I-2655 | I-2656 |
| I-2657 | I-2658 | I-2659 | I-2660 | I-2661 |
| I-2662 | I-2663 | I-2664 | I-2665 | I-2666 |
| I-2667 | I-2668 | I-2669 | I-2670 | I-2671 |
| I-2672 | I-2673 | I-2674 | I-2675 | I-2676 |
| I-2677 | I-2678 | I-2679 | I-2680 | I-2682 |
| I-2684 | I-2685 | I-2686 | I-2687 | I-2688 |
| I-2689 | I-2690 | I-2691 | I-2692 | I-2693 |
| I-2696 | I-2697 | I-2698 | I-2699 | I-2700 |
| I-2708 | I-2709 | I-2710 | I-2711 | I-2712 |
| I-2713 | I-2714 | I-2715 | I-2716 | I-2717 |
| I-2718 | I-2719 | I-2720 | I-2721 | I-2722 |
| I-2723 | I-2724 | I-2725 | I-2726 | I-2727 |
| I-2728 | I-2729 | I-2730 | I-2731 | I-2732 |
| I-2733 | I-2734 | I-2735 | I-2736 | I-2737 |
| I-2738 | I-2739 | I-2740 | I-2741 | I-2742 |
| I-2743 | I-2744 | I-2745 | I-2746 | I-2747 |
| I-2752 | I-2753 | I-2754 | I-2755 | I-2757 |
| I-2758 | I-2759 | I-2760 | I-2761 | I-2762 |
| I-2763 | I-2770 | I-2771 | I-2772 | I-2773 |
| I-2774 | I-2775 | I-2777 | I-2778 | I-2779 |
| I-2780 | I-2781 | I-2782 | I-2783 | I-2784 |
| I-2785 | I-2786 | I-2787 | I-2788 | I-2789 |
| I-2790 | I-2791 | I-2792 | I-2793 | I-2794 |
| I-2795 | I-2796 | I-2797 | I-2798 | I-2799 |
| I-2801 | I-2802 | I-2803 | I-2804 | I-2805 |
| I-2808 | I-2809 | I-2810 | I-2811 | I-2812 |
| I-2813 | I-2814 | I-2815 | I-2816 | I-2817 |
| I-2818 | I-2823 | I-2824 | I-2825 | I-2826 |
| I-2827 | I-2828 | I-2829 | I-2830 | I-2831 |
| I-2832 | I-2833 | I-2834 | I-2835 | I-2836 |
| I-2837 | I-2838 | I-2839 | I-2840 | I-2843 |
| I-2844 | I-2845 | I-2846 | I-2857 | I-2858 |
| I-2859 | I-2860 | I-2861 | I-2862 | I-2863 |
| I-2864 | I-2865 | I-2866 | I-2867 | I-2868 |
| I-2869 | I-2870 | I-2871 | I-2872 | I-2873 |
| I-2874 | I-2875 | I-2876 | I-2877 | I-2878 |
| I-2879 | I-2880 | I-2881 | I-2884 | I-2885 |
| I-2886 | I-2887 | I-2888 | I-2889 | I-2890 |
| I-2891 | I-2892 | I-2893 | I-2894 | I-2895 |
| I-2902 | I-2903 | I-2904 | I-2905 | I-2906 |
| I-2915 | I-2916 | I-2917 | I-2918 | I-2919 |
| I-2920 | I-2921 | I-2922 | I-2923 | I-2925 |
| I-2926 | I-2927 | I-2928 | I-2929 | I-2942 |
| I-2943 | I-2944 | I-2945 | I-2946 | I-2947 |
| I-2948 | I-2949 | I-2950 | I-2951 | I-2952 |
| I-2953 | I-2954 | I-2955 | I-2956 | I-2957 |
| I-2958 | I-2959 | I-2960 | I-2961 | I-2962 |
| I-2963 | I-2964 | I-2965 | I-2966 | I-2967 |
| I-2973 | I-2974 | I-2975 | I-2976 | I-2977 |
| I-2978 | I-2979 | I-2980 | I-2981 | I-2982 |
| I-2983 | I-2984 | I-2985 | I-2989 | I-2990 |
| I-2991 | I-2996 | I-2997 | I-2998 | I-2999 |
| I-3000 | I-3001 | I-3002 | I-3003 | I-3004 |
| I-3005 | I-3006 | I-3007 | I-3008 | I-3009 |
| I-3010 | I-3011 | I-3012 | I-3013 | I-3014 |
| I-3015 | I-3016 | I-3017 | I-3018 | I-3019 |
| I-3020 | I-3021 | I-3022 | I-3023 | I-3024 |
| I-3025 | I-3026 | I-3027 | I-3028 | I-3029 |
| I-3044 | I-3045 | I-3046 | I-3047 | I-3048 |
| I-3049 | I-3050 | I-3051 | I-3052 | I-3053 |
| I-3054 | I-3055 | I-3056 | I-3057 | I-3058 |
| I-3059 | I-3060 | I-3061 | I-3062 | I-3063 |
| I-3064 | I-3065 | I-3067 | I-3068 | I-3069 |
| I-3070 | I-3071 | I-3073 | I-3081 | I-3082 |
| I-3083 | I-3084 | I-3085 | I-3086 | I-3087 |
| I-3088 | I-3089 | I-3091 | I-3092 | I-3093 |
| I-3094 | I-3095 | I-3096 | I-3098 | I-3099 |
| I-3100 | I-3101 | I-3102 | I-3103 | I-3104 |
| I-3105 | I-3106 | I-3109 | I-3110 | I-3111 |
| I-3112 | I-3113 | I-3115 | I-3118 | I-3119 |
| I-3120 | I-3121 | I-3122 | I-3123 | I-3124 |
| I-3125 | I-3126 | I-3127 | I-3128 | I-3129 |
| I-3130 | I-3131 | I-3132 | I-3134 | I-3137 |
| I-3138 | I-3139 | I-3140 | I-3141 | I-3144 |
| I-3145 | I-3146 | I-3147 | I-3148 | I-3149 |
| I-3150 | I-3151 | I-3152 | I-3153 | I-3154 |
| I-3156 | I-3157 | I-3160 | I-3161 | I-3164 |
| I-3166 | I-3167 | I-3168 | I-3169 | I-3170 |
| I-3171 | I-3172 | I-3173 | I-3174 | I-3175 |
| I-3176 | I-3177 | I-3178 | I-3179 | I-3180 |
| I-3181 | I-3182 | I-3183 | I-3184 | I-3185 |
| I-3186 | I-3187 | I-3188 | I-3189 | I-3190 |
| I-3191 | I-3192 | I-3193 | I-3194 | I-3195 |
| I-3202 | I-3203 | I-3204 | I-3212 | I-3213 |
| I-3214 | I-3215 | I-3216 | I-3217 | I-3218 |
| I-3219 | I-3220 | I-3221 | I-3222 | I-3223 |
| I-3224 | I-3225 | I-3226 | I-3227 | I-3228 |
| I-3232 | I-3233 | I-3234 | I-3235 | I-3238 |
| I-3239 | I-3240 | I-3241 | I-3242 | I-3243 |
| I-3244 | I-3245 | I-3246 | I-3247 | I-3248 |
| I-3249 | I-3250 | I-3251 | I-3252 | I-3253 |
| I-3254 | I-3255 | I-3256 | I-3257 | I-3258 |
| I-3259 | I-3260 | I-3261 | I-3262 | I-3263 |
| I-3264 | I-3265 | I-3266 | I-3267 | I-3268 |
| I-3269 | I-3273 | I-3274 | I-3276 | I-3277 |
| I-3278 | I-3279 | I-3280 | I-3281 | I-3282 |
| I-3283 | I-3284 | I-3285 | I-3286 | I-3287 |
| I-3288 | I-3289 | I-3290 | I-3291 | I-3292 |
| I-3293 | I-3294 | I-3297 | I-3298 | I-3316 |
| I-3317 | I-3318 | I-3319 | I-3320 | I-3321 |
| I-3324 | I-3325 | I-3326 | I-3327 | I-3328 |
| I-3329 | I-3331 | I-3332 | I-3333 | I-3334 |
| I-3335 | I-3336 | I-3337 | I-3338 | I-3339 |
| I-3340 | I-3341 | I-3342 | I-3350 | I-3351 |
| I-3352 | I-3353 | I-3354 | I-3355 | I-3356 |
| I-3357 | I-3358 | I-3359 | I-3367 | I-3373 |
| I-3374 | I-3375 | I-3376 | I-3377 | I-3378 |
| I-3379 | I-3380 | I-3381 | I-3382 | I-3383 |
| I-3384 | I-3385 | I-3386 | I-3387 | I-3388 |
| I-3389 | I-3390 | I-3391 | I-3392 | I-3393 |
| I-3394 | I-3395 | I-3396 | I-3398 | I-3401 |
| I-3402 | I-3403 | I-3404 | I-3405 | I-3406 |
| I-3408 | I-3409 | I-3410 | I-3411 | I-3414 |
| I-3415 | I-3416 | I-3417 | I-3423 | I-3425 |
| I-3426 | I-3427 | I-3428 | I-3429 | I-3430 |
| I-3431 | I-3432 | I-3433 | I-3434 | I-3435 |
| I-3436 | I-3437 | I-3438 | I-3441 | I-3442 |
| I-3443 | I-3444 | I-3449 | I-3450 | I-3451 |
| I-3479 | I-3487 | I-3488 | I-3489 | I-3490 |
| I-3491 | I-3492 | I-3493 | I-3494 | I-3495 |
| I-3496 | I-3497 | I-3498 | I-3499 | I-3500 |
| I-3501 | I-3502 | I-3503 | I-3504 | I-3505 |
| I-3506 | I-3507 | I-3508 | I-3509 | I-3510 |
| I-3511 | I-3512 | I-3513 | I-3514 | I-3515 |
| I-3516 | I-3518 | I-3519 | I-3521 | I-3522 |
| I-3526 | I-3527 | I-3528 | I-3529 | I-3530 |
| I-3531 | I-3532 | I-3533 | I-3535 | I-3536 |
| I-3537 | I-3538 | I-3539 | I-3540 | I-3541 |
| I-3544 | I-3545 | I-3546 | I-3547 | I-3548 |
| I-3586 | I-3587 | I-3588 | I-3589 | I-3590 |
| I-3591 | I-3592 | I-3593 | I-3595 | I-3596 |
| I-3597 | I-3598 | I-3599 | I-3600 | I-3605 |
| I-3606 | I-3607 | I-3608 | I-3609 | I-3610 |
| I-3615 | I-3616 | I-3617 | I-3618 | I-3619 |
| I-3620 | I-3621 | I-3622 | I-3624 | I-3626 |
| I-3627 | I-3629 | I-3630 | I-3634 | I-3635 |
| I-3636 | I-3637 | I-3638 | I-3639 | I-3640 |
| I-3641 | I-3642 | I-3643 | I-3644 | I-3645 |
| I-3646 | I-3647 | I-3648 | I-3649 | I-3650 |
| I-3671 | I-3673 | I-3678 | I-3679 | I-3680 |
| I-3682 | I-3683 | I-3688 | I-3689 | I-3691 |
| I-3692 | I-3693 | I-3694 | I-3695 | I-3697 |
| I-3698 | I-3699 | I-3700 | I-3701 | I-3702 |
| I-3703 | I-3704 | I-3705 | I-3706 | I-3707 |
| I-3709 | I-3710 | I-3712 | I-3713 | I-3717 |
| I-3718 | I-3721 | I-3723 | I-3724 | I-3727 |
| I-3728 | I-3729 | I-3730 | I-3731 | I-3732 |
| I-3733 | I-3734 | I-3735 | I-3736 | I-3737 |
| I-3738 | I-3739 | I-3740 | | |

### Example 33

### rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid

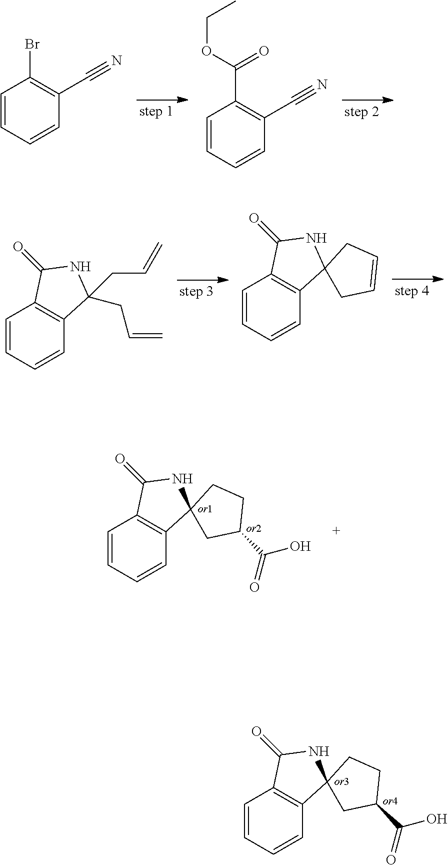 [SMILES: N#Cc1ccccc1Br ; CCOC(=O)c1ccccc1C#N ; C=CCC1(CC=C)NC(=O)c2ccccc21 ; O=C1NC2(CC=CC2)c2ccccc21 ; O=C1N[C@]2(CC[C@H](C(=O)O)C2)c2ccccc21 ; O=C1N[C@]2(CC[C@@H](C(=O)O)C2)c2ccccc21]

### Step 1. Synthesis of ethyl 2-cyanobenzoate

A 50 mL flame dried flask was charged with 2-bromobenzonitrile (1.00 g, 5.49 mmol). Dry THF (50 mL) was added under a N₂ atmosphere and the reaction media was cooled down to −78° C. n-butyllithium (2.64 mL, 2.5 molar in THF, 6.59 mmol) was added dropwise and the reaction was stirred for 15 minutes. Ethyl carbonocyanidate (651 μL, 6.59 mmol) was added and the reaction was slowly warmed up to room temperature. After 1 hour the reaction was quenched with saturated NH₄Cl solution. The aqueous layer was extracted twice with EtOAc. The organic layers were combined and washed once with water, then dried over Na₂SO₄ and concentrated under reduced pressure. The material was purified by normal phase column chromatography (hexanes:EtOAc 100:0 to 80:20) to give ethyl 2-cyanobenzoate (396 mg) as a viscous colorless oil. 1H NMR: (400 MHz, CDCl₃) δ 8.12-8.08 (m, 1H), 7.76 (dd, J=7.0, 1.6 Hz, 1H), 7.67-7.59 (m, 2H), 4.42 (q, J=7.1 Hz, 2H), 1.40 (t, J=7.1 Hz, 3H).

### Step 2. Synthesis of 3,3-diallylisoindolin-1-one

To a suspension of zinc dust (224 mg, 3.42 mmol) in THF (5 mL) were successively added ethyl 2-cyanobenzoate (200 mg, 1.14 mmol) in THF (0.5 mL) and allyl bromide (294 μL, 3.42 mmol). The solution was heated to reflux and cooled down to room temperature after 15 min of heating. HCl (1 N, 5 mL) was added to the reaction and the aqueous layer was extracted with EtOAc (2×5 mL). The combined organic layers were washed with 2 N NaOH and brine, dried over anhydrous Na₂SO₄ and filtered. The solvent was removed in vacuo to afford 3,3-diallylisoindolin-1-one (240 mg, crude) as a yellow viscous oil. LCMS RT 1.39 min, [M+H]⁺ 214.1, LCMS method L.

### Step 3. Synthesis of spiro[cyclopentane-1,1′-isoindolin]-3-en-3′-one

A 150 mL flame-dried flask was charged with 3,3-diallylisoindolin-1-one (240 mg, 1.13 mmol). Dry toluene (40 mL) was added under a N₂ atmosphere and the reaction was heated to 70° C. Benzylidene(dichloro)(1,3-dimesityl-2-imidazolidinylidene)ruthenium-tricyclohexylphosphine (1:1) (47.8 mg, 56.3 μmol) was added and the reaction was kept at this temperature for 90 min. After cooling to room temperature, the reaction media was concentrated under reduced pressure and the crude material was purified using normal phase column chromatography (DCM:EtOAc 100:0 to 50:50) to give spiro[cyclopentane-1,1′-isoindolin]-3-en-3′-one (98 mg) as a colorless oil. ¹H NMR (400 MHz, CDCl₃) δ 7.79 (d, J=7.5 Hz, 1H), 7.60-7.51 (m, 2H), 7.50-7.40 (m, 2H), 5.90 (d, J=8.2 Hz, 1H), 5.88 (d, J=8.3 Hz, 1H), 2.89 (d, J=15.8 Hz, 1H), 2.89 (d, J=15.8 Hz, 1H), 2.77 (d, J=15.7 Hz, 1H). LCMS RT 1.23 min, [M+H]⁺ 186.1, LCMS method L.

### Step 4. Synthesis of rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid

To a flame dried 10 mL flask was added palladium diacetate (6.06 mg, 27.0 μmol) and 4,5-bis(diphenylphosphino)-9,9-dimethyl xanthene (15.6 mg, 27.0 μmol). Dry PhMe (0.2 mL) was added under a N₂ atmosphere followed by spiro[cyclopentane-1,1′-isoindolin]-3-en-3′-one (100 mg, 540 μmol), formic acid (41.2 μL, 1.08 mmol), and acetic anhydride (10.2 μL, 108 μmol) successively via syringe. The vial was purged with N₂ and tightly sealed with a septum cap. The reaction mixture was stirred at 70° C. for 24 hours. The reaction media was cooled down to room temperature and diluted with DCM and HCl (1 N). The aqueous layer was extracted with DCM 3 times. The organic layers were dried over Na₂SO₄ and concentrated under reduced pressure. The crude material was diluted in DMF and purified on a 30 g C18 column (mobile phase A: 10 mM ammonium formate in water, mobile phase B: acetonitrile, gradient: A:B 95:5 to 70:30) to give rac-(1R,3R)-3′-oxospiro[cyclopentane-1,1′-isoindoline]-3-carboxylic acid and rac-(1R,3S)-3′-oxospiro[cyclopentane-1,1′-isoindoline]-3-carboxylic acid. Isomer 1: 28 mg, LCMS RT 1.04 min, [M+H]⁺ 232.0, LCMS method L. Isomer 2: 29 mg, LCMS RT 1.09 min, [M+H]⁺ 232.0, LCMS method L.

Additional compounds prepared according to the methods of Example 33 are listed in Table 3 below. Corresponding ¹H NMR and mass spectrometry characterization for these compounds are described in Table 1. Certain compounds in Table 3 below were prepared with other compounds whose preparation is described further below in the Examples.

TABLE 3
Additional exemplary compounds

| | | | | |
|---|---|---|---|---|
| I-1898 | I-1905 | I-1982 | I-1983 | I-2022 |
| I-2023 | I-2024 | I-2025 | I-2026 | I-2027 |
| I-2028 | I-2029 | I-2120 | I-2121 | I-2122 |
| I-2123 | I-2124 | I-2125 | I-2126 | I-2127 |

### Example 34

### (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide

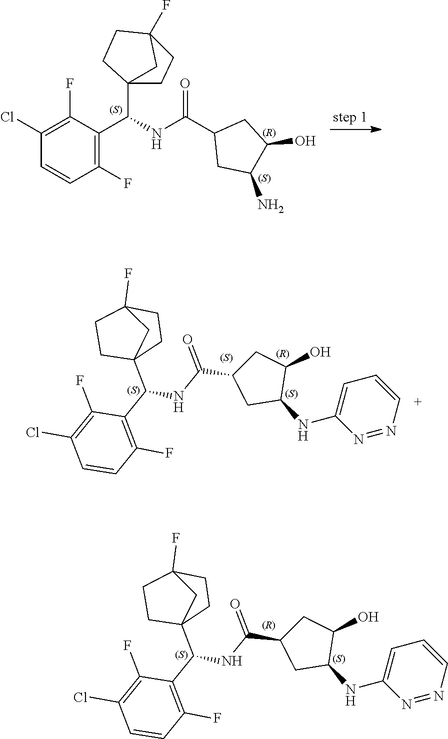

### Step 1. Synthesis of (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide

A mixture of (3S,4R)-3-amino-N—((S)-(3-chloro-2,6-difluorophenyl)(4-fluorobicyclo[2.2.1]heptan-1-yl)methyl)-4-hydroxycyclopentane-1-carboxamide (100 mg, 240 μmol), 3-bromopyridazine (45.8 mg, 288 μmol), Cs₂CO₃ (235 mg, 720 μmol) and Pd-PEPPSI-IHept-Cl (46.7 mg, 48.0 μmol) in 1,4-dioxane (5 mL) was stirred for 6 hours at 90° C. under a N₂ atmosphere. The resulting crude material was purified by preparative HPLC (column: XBridge Prep OBD C18 Column, 30*150 mm, 5 μm; mobile phase A: water (10 mmol/L NH₄HCO₃+0.05% NH₄OH), mobile phase B: acetonitrile; flow rate: 60 mL/min; gradient: 42% B to 62% B in 8 min; wavelength: 220 nm; RT (min): 8.52) to give an off-white solid (50 mg, 42%). The solid was further purified by chiral HPLC (column: CHIRALPAKIG3; mobile phase A: hexane (0.2% diethylamine); mobile phase B: (EtOH:DCM 1:1); flow rate: 1 mL/min; gradient: isocratic; injection volume: 3 mL) to give (1S,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide and (1R,3R,4S)—N—((S)-(3-chloro-2,6-difluorophenyl)(4-fluorobicyclo[2.2.1]heptan-1-yl)methyl)-3-hydroxy-4-(pyridazin-3-ylamino)cyclopentane-1-carboxamide, both as an amorphous off-white solid. Peak 1: 10.9 mg (22.0 μmol). ¹H NMR (400 MHz, DMSO-d6) δ 8.37 (dd, J=4.3, 1.4 Hz, 1H), 8.24 (d, J=8.2 Hz, 1H), 7.56 (td, J=8.7, 5.4 Hz, 1H), 7.22-7.10 (m, 2H), 6.87 (dd, J=9.0, 1.5 Hz, 1H), 6.47 (d, J=7.0 Hz, 1H), 5.27 (d, J=8.2 Hz, 1H), 4.88 (d, J=3.7 Hz, 1H), 4.17 (d, J=5.5 Hz, 2H), 3.15 (qd, J=8.6, 6.0 Hz, 1H), 1.93-1.85 (m, 2H), 1.84-1.74 (m, 6H), 1.71 (d, J=12.3 Hz, 3H), 1.60 (d, J=8.4 Hz, 1H), 1.46 (d, J=9.1 Hz, 2H). LCMS RT 0.94 min, [M+H]⁺ 495, LCMS method D; Peak 2: 20.0 mg (40.4 μmol)¹H NMR (400 MHz, DMSO-d6) δ 8.38 (dd, J=4.4, 1.4 Hz, 1H), 8.24 (d, J=8.2 Hz, 1H), 7.57 (td, J=8.7, 5.4 Hz, 1H), 7.30-7.04 (m, 2H), 6.90 (dd, J=9.1, 1.4 Hz, 1H), 6.50 (d, J=7.5 Hz, 1H), 5.29 (d, J=8.2 Hz, 1H), 4.88 (d, J=3.6 Hz, 1H), 4.28-4.10 (m, 2H), 3.14 (dd, J=12.7, 7.9 Hz, 1H), 2.02 (ddd, J=12.7, 8.1, 4.8 Hz, 1H), 1.90-1.78 (m, 4H), 1.77-1.65 (m, 6H), 1.60 (d, J=8.4 Hz, 1H), 1.46 (d, J=8.6 Hz, 2H). LCMS RT 0.94 min, [M+H]⁺ 495, LCMS method D.

Additional compounds prepared according to the methods of Example 34 are listed in Table 4 below. Corresponding ¹H NMR and mass spectrometry characterization for these compounds are described in Table 1. Certain compounds in Table 4 below were prepared with other compounds whose preparation is described further below in the Examples.

TABLE 4
Additional exemplary compounds

| | | | | |
|---|---|---|---|---|
| I-2015 | I-2016 | I-2094 | I-2108 | I-2111 |
| I-2128 | I-2130 | I-2132 | I-2135 | I-2156 |
| I-2159 | I-2160 | I-2221 | I-2312 | I-2315 |
| I-2321 | I-2324 | I-2343 | I-2800 | I-2806 |
| I-2807 | I-2841 | I-2842 | I-2924 | I-3439 |
| I-3440 | I-3542 | I-3543 | I-3623 | I-3625 |
| I-3628 | I-3674 | I-3675 | I-3690 | I-3696 |
| I-3711 | I-3719 | I-3725 | I-3726 | |

### Example 35

### (S)-1-(1-acetyl-4-methylpiperidin-4-yl)-3-((3-chlorophenyl)(cyclopentyl)methyl)urea

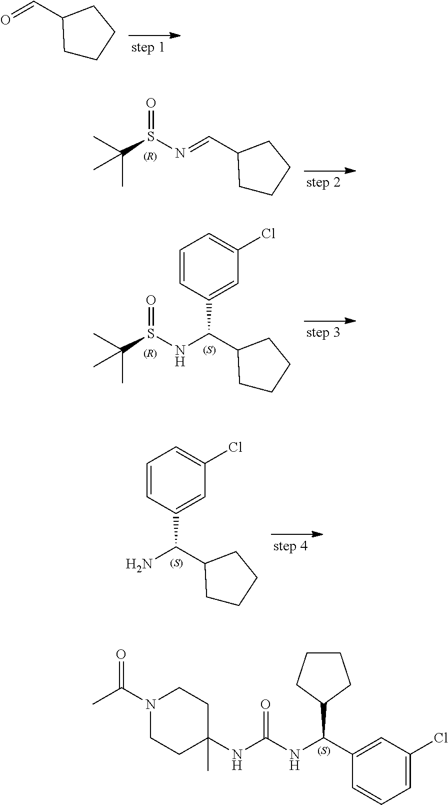

### Step 1. Synthesis of (R)—N-(cyclopentylmethylene)-2-methylpropane-2-sulfinamide

To a solution of cyclopentanecarbaldehyde (112 g, 1.15 mol) and (R)-2-methylpropane-2-sulfinamide (167 g, 1.38 mol) in THF (560 mL) was added Ti(OⁱPr)₄ (651 g, 2.29 mol) under a N₂ atmosphere at 25° C. The mixture was heated to 75° C. and stirred for 2 hours. After cooling to room temperature, to the mixture was added brine (3.00 L). The suspension was filtered. The filter cake was washed with ethyl acetate (5.00 L*2). The organic phase in the filtrate was separated and the aqueous phase was extracted with ethyl acetate (3.00 L). The combined organic phase was washed with brine (3.0 L), dried over Na₂SO₄, filtered and concentrated. The residue was purified by column chromatography (silica gel, petroleum ether:ethyl acetate 1:0 to 10:1) to give (R)—N-(cyclopentylmethylene)-2-methylpropane-2-sulfinamide (357 g, 1.77 mol) as a yellow oil. ¹H NMR (400 MHz CDCl₃)

δ 8.00 (d, J=5.6 Hz, 1H), 2.98-2.94 (m, 1H), 1.94-1.83 (m, 2H), 1.77-1.62 (m, 6H), 1.19 (s, 9H).

### Step 2. Synthesis of (R)—N—((S)-(3-chlorophenyl)(cyclopentyl)methyl)-2-methylpropane-2-sulfinamide

Two batches were executed. To a solution of (R)—N-(cyclopentylmethylene)-2-methylpropane-2-sulfinamide (160 g, 795 mmol) and 1-bromo-3-chlorobenzene (140 mL, 1.19 mol) in THF (800 mL) was added n-BuLi (2.50 M in THF, 477 mL) dropwise at −60˜−70° C. under N₂. The reaction was stirred between −70 and −60° C. for 2 hours. Two batches of mixture were combined. The mixture was poured into saturated NH₄Cl solution (5.0 L) and extracted with ethyl acetate (2.00 L*3). Then the combined organic phase was washed with brine (2.00 L), dried over Na₂SO₄, filtered and concentrated to give the crude product (R)—N—((S)-(3-chlorophenyl)(cyclopentyl)methyl)-2-methylpropane-2-sulfinamide as a yellow oil (563 g), which was used in the next step without purification.

### Step 3. Synthesis of (S)-(3-chlorophenyl)(cyclopentyl)methanamine

Two batches were carried out in parallel. To a solution of (R)—N—((S)-(3-chlorophenyl)(cyclopentyl)methyl)-2-methylpropane-2-sulfinamide (264 g, 757 mmol) in ethyl acetate (2.60 L) was added HCl (4 N in EtOAc, 473 mL) at 25° C. The mixture was stirred at 25° C. for 1 hour. After 1 hour of stirring, a large amount of white solid was formed. Two batches of reaction mixture were combined. The suspension was concentrated to 4.0 L. The suspension was filtered and the filter cake was washed with ethyl acetate (200 mL*2). Then the filter cake was partitioned between ethyl acetate (2.00 L) and saturated NaHCO₃ solution (2.50 L). The suspension was stirred for 10 minutes until the solid disappeared. The organic phase was separated and the aqueous phase was extracted with ethyl acetate (1.00 L*2). The combined organic phase was washed with brine (2.00 L), dried over Na₂SO₄, filtered and concentrated to give the crude product (S)-(3-chlorophenyl)(cyclopentyl)methanamine (220 g) as a yellow oil, which was used in the next step without purification.

### Step 4. Synthesis of (S)-1-(1-acetyl-4-methylpiperidin-4-yl)-3-((3-chlorophenyl)(cyclopentyl)methyl)urea

(3-chlorophenyl)(cyclopentyl)methanamine (100 mg, 477 μmol) was dissolved in DCM (5 mL). The solution was cooled to 0° C. CDI (92.8 mg, 572 μmol) was added, followed by DMAP (5.83 mg, 47.7 μmol). The solution was stirred at 0° C. for 1 hour. 1-(4-amino-4-methylpiperidin-1-yl)ethan-1-one hydrochloride (91.9 mg, 477 μmol) and triethylamine (199 μL, 1.43 mmol) were added, and the solution was stirred at 40° C. for 1.5 h, and then at room temperature overnight. It was then heated at 40° C. for 4.5 h, concentrated and purified by HPLC to give the product 1-(1-acetyl-4-methylpiperidin-4-yl)-3-((3-chlorophenyl)(cyclopentyl)methyl)urea (53.1 mg, 135 μmol) as a colorless solid. LCMS: RT 1.410 min, [M+H]⁺392.46, LCMS method I.

### Example 36

### (1R,3R)—N—((R)-(2,3-dichloro-6-fluorophenyl)(1-methylcyclopentyl)methyl)-3-(3-methylureido)cyclopentane-1-carboxamide

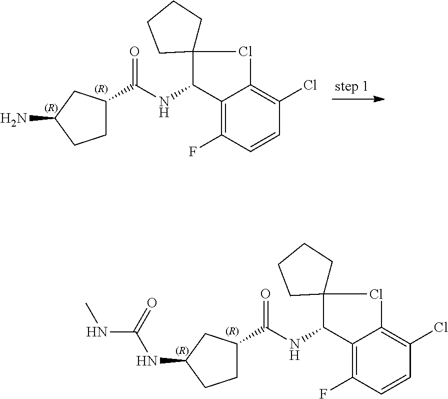

### Step 1. Synthesis of (1R,3R)—N—((R)-(2,3-dichloro-6-fluorophenyl)(1-methylcyclopentyl)methyl)-3-(3-methylureido)cyclopentane-1-carboxamide

A mixture of (1R,3R)-3-amino-N—((R)-(2,3-dichloro-6-fluorophenyl)(1-methylcyclopentyl)methyl)cyclopentane-1-carboxamide (50 mg, 0.13 mmol), N-methyl-1H-imidazole-1-carboxamide (16 mg, 0.13 mmol) and TEA (26 mg, 0.26 mmol) in CH₃CN (1 mL) was stirred for 2 h at 25° C. The reaction was quenched with MeOH (1 mL) and concentrated. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 35% B to 65% B in 8 min, then 65% B; wavelength: 220 nm; RT1 (min): 7.53) to give (1R,3R)—N—((R)-(2,3-dichloro-6-fluorophenyl)(1-methylcyclopentyl)methyl)-3-(3-methylureido)cyclopentane-1-carboxamide (17.4 mg, 39.2 μmol) as an off-white solid. ¹H NMR (400 MHz, DMSO-d₆) δ 8.05 (d, J=8.7 Hz, 1H), 7.60 (d, J=9.6 Hz, 1H), 7.25 (t, J=9.8 Hz, 1H), 5.81 (d, J=7.1 Hz, 1H), 5.56 (s, 1H), 5.49 (d, J=8.6 Hz, 1H), 3.88 (q, J=6.5 Hz, 1H), 2.99-2.91 (m, 1H), 2.53 (s, 3H), 1.88-1.70 (m, 3H), 1.68-1.57 (m, 7H), 1.44 (dd, J=12.6, 7.5 Hz, 1H), 1.37 (s, 1H), 1.35-1.24 (m, 2H), 0.96 (d, J=2.8 Hz, 3H). LCMS RT 1.158 min, [M+H]⁺ 444, LCMS method C.

### Example 37

### N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide

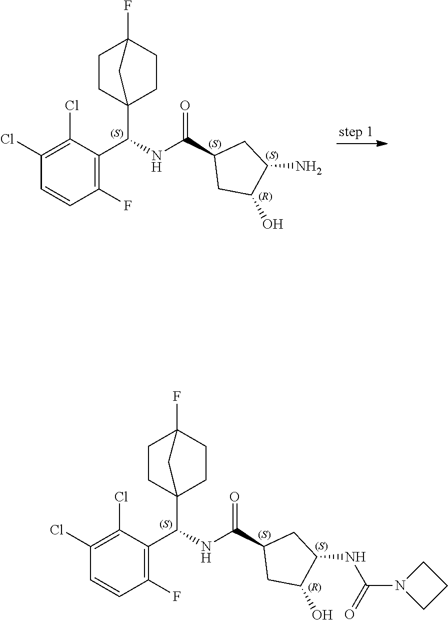

### Step 1. Synthesis of N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide

(1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide was synthesized similarly as example 5. To a stirred solution of azetidine (13 mg, 231 μmol) and TEA (70 mg, 692 μmol) in CH₂Cl₂ (3 mL) was added triphosgene (20 mg, 0.30 Eq, 69.2 μmol) dropwise at 0° C. under a nitrogen atmosphere. The resulting mixture was stirred for 1 hour at 30° C. under nitrogen. To the above mixture was added (1S,3S,4R)-3-amino-N—((S)-(2,3-dichloro-6-fluorophenyl)(4-fluorobicyclo[2.2.1]heptan-1-yl)methyl)-4-hydroxycyclopentane-1-carboxamide (100 mg, 231 μmol) at room temperature. The resulting mixture was stirred for 1 hour at room temperature. The resulting mixture was purified by reversed-phase flash chromatography (column: XBridge Prep OBD C18 Column, 30*150 mm, 10 μm; mobile phase A: water (10 mM NH₄HCO₃+0.05% NH₄OH), mobile phase B: Acetonitrile; flow rate: 60 mL/min; gradient: 32% B to 49% B in 8 minutes; wavelength: 254 nm/220 nm; RT (min): 9.48) to give N-((1S,2R,4S)-4-(((S)-(2,3-dichloro-6-fluorophenyl)(4-fluorobicyclo[2.2.1]heptan-1-yl)methyl)carbamoyl)-2-hydroxycyclopentyl)azetidine-1-carboxamide (4.3 mg, 8.0 μmol) as a white solid.

LCMS RT 1.389 min, [M+H]⁺ 516.20. LCMS Method F. ¹H NMR (300 MHz, DMSO-d6) δ 8.17 (d, J=8.2 Hz, 1H), 7.62 (dd, J=8.9, 5.1 Hz, 1H), 7.26 (dd, J=10.6, 9.0 Hz, 1H), 5.49 (d, J=7.7 Hz, 2H), 4.77 (d, J=3.5 Hz, 1H), 3.91-3.85 (m, 1H), 3.78 (dd, J=9.9, 5.1 Hz, 4H), 3.12-2.98 (m, 1H), 2.15-2.07 (m, 2H), 1.85-1.49 (in, 15H). ¹⁹F NMR (282 MHz, DMSO) δ −109.27, −173.53, −173.77.

Additional compounds prepared according to the methods of Examples 35-37 are listed in Table 5 below. Corresponding ¹H NMR and mass spectrometry characterization for these compounds are described in Table 1. Certain compounds in Table 5 below were prepared with other compounds whose preparation is described further below in the Examples.

TABLE 5
Additional Exemplary Compounds

| | | | | |
|---|---|---|---|---|
| I-1 | I-2 | I-4 | I-5 | I-7 |
| I-8 | I-9 | I-10 | I-11 | I-12 |
| I-13 | I-14 | I-15 | I-16 | I-17 |
| I-18 | I-19 | I-20 | I-21 | I-22 |
| I-23 | I-24 | I-25 | I-26 | I-27 |
| I-28 | I-29 | I-30 | I-31 | I-32 |
| I-33 | I-34 | I-35 | I-36 | I-37 |
| I-38 | I-39 | I-40 | I-41 | I-42 |
| I-43 | I-44 | I-45 | I-46 | I-47 |
| I-48 | I-49 | I-50 | I-51 | I-52 |
| I-53 | I-54 | I-55 | I-56 | I-57 |
| I-58 | I-59 | I-60 | I-61 | I-62 |
| I-63 | I-64 | I-65 | I-66 | I-67 |
| I-68 | I-69 | I-70 | I-71 | I-72 |
| I-73 | I-74 | I-75 | I-76 | I-77 |
| I-78 | I-79 | I-80 | I-81 | I-82 |
| I-84 | I-85 | I-86 | I-87 | I-88 |
| I-89 | I-90 | I-91 | I-92 | I-93 |
| I-94 | I-95 | I-96 | I-97 | I-98 |
| I-99 | I-100 | I-101 | I-102 | I-103 |
| I-104 | I-105 | I-106 | I-107 | I-108 |
| I-109 | I-110 | I-111 | I-112 | I-113 |
| I-114 | I-115 | I-116 | I-117 | I-118 |
| I-119 | I-120 | I-121 | I-122 | I-123 |
| I-124 | I-125 | I-126 | I-127 | I-128 |
| I-129 | I-130 | I-131 | I-132 | I-133 |
| I-134 | I-135 | I-136 | I-137 | I-138 |
| I-139 | I-140 | I-141 | I-142 | I-143 |
| I-144 | I-145 | I-146 | I-147 | I-148 |
| I-149 | I-150 | I-151 | I-152 | I-153 |
| I-154 | I-155 | I-156 | I-157 | I-158 |
| I-159 | I-160 | I-161 | I-162 | I-163 |
| I-164 | I-165 | I-166 | I-167 | I-168 |
| I-169 | I-170 | I-171 | I-172 | I-173 |
| I-174 | I-175 | I-176 | I-177 | I-178 |
| I-179 | I-182 | I-184 | I-185 | I-186 |
| I-187 | I-188 | I-189 | I-190 | I-195 |
| I-196 | I-197 | I-204 | I-205 | I-206 |
| I-207 | I-215 | I-219 | I-220 | I-221 |
| I-223 | I-225 | I-230 | I-231 | I-232 |
| I-233 | I-235 | I-254 | I-255 | I-256 |
| I-261 | I-262 | I-264 | I-265 | I-267 |
| I-268 | I-274 | I-275 | I-276 | I-277 |
| I-278 | I-279 | I-280 | I-281 | I-282 |
| I-283 | I-284 | I-285 | I-286 | I-287 |
| I-288 | I-289 | I-290 | I-291 | I-292 |
| I-293 | I-299 | I-300 | I-301 | I-302 |
| I-303 | I-304 | I-305 | I-306 | I-307 |
| I-308 | I-309 | I-310 | I-311 | I-312 |
| I-313 | I-314 | I-315 | I-316 | I-317 |
| I-318 | I-319 | I-320 | I-321 | I-322 |
| I-323 | I-324 | I-326 | I-328 | I-329 |
| I-330 | I-331 | I-332 | I-335 | I-336 |
| I-345 | I-346 | I-347 | I-350 | I-355 |
| I-356 | I-357 | I-359 | I-361 | I-363 |
| I-365 | I-368 | I-374 | I-379 | I-380 |
| I-381 | I-382 | I-383 | I-385 | I-386 |
| I-387 | I-388 | I-389 | I-390 | I-391 |
| I-392 | I-393 | I-394 | I-395 | I-396 |
| I-397 | I-398 | I-399 | I-400 | I-401 |
| I-402 | I-404 | I-405 | I-407 | I-408 |
| I-409 | I-410 | I-412 | I-413 | I-414 |
| I-415 | I-416 | I-417 | I-418 | I-419 |
| I-420 | I-421 | I-422 | I-423 | I-425 |
| I-426 | I-428 | I-431 | I-433 | I-434 |
| I-435 | I-436 | I-443 | I-444 | I-447 |
| I-448 | I-449 | I-450 | I-451 | I-452 |
| I-453 | I-456 | I-457 | I-458 | I-459 |
| I-462 | I-463 | I-464 | I-465 | I-468 |
| I-472 | I-473 | I-474 | I-480 | I-481 |
| I-482 | I-483 | I-486 | I-487 | I-495 |
| I-496 | I-497 | I-499 | I-500 | I-501 |
| I-502 | I-503 | I-516 | I-517 | I-518 |
| I-519 | I-520 | I-523 | I-529 | I-530 |
| I-531 | I-532 | I-533 | I-534 | I-535 |
| I-548 | I-565 | I-584 | I-585 | I-586 |
| I-587 | I-588 | I-589 | I-590 | I-591 |
| I-592 | I-593 | I-594 | I-595 | I-596 |
| I-597 | I-599 | I-601 | I-602 | I-603 |
| I-604 | I-605 | I-617 | I-618 | I-623 |
| I-624 | I-625 | I-626 | I-627 | I-628 |
| I-629 | I-631 | I-632 | I-633 | I-636 |
| I-637 | I-638 | I-646 | I-647 | I-648 |
| I-649 | I-653 | I-654 | I-655 | I-656 |
| I-662 | I-663 | I-664 | I-677 | I-697 |
| I-698 | I-699 | I-705 | I-706 | I-707 |
| I-708 | I-713 | I-714 | I-715 | I-716 |
| I-720 | I-721 | I-722 | I-723 | I-726 |
| I-727 | I-728 | I-729 | I-731 | I-732 |
| I-733 | I-734 | I-735 | I-736 | I-737 |
| I-738 | I-739 | I-740 | I-741 | I-742 |
| I-743 | I-749 | I-750 | I-751 | I-752 |
| I-753 | I-754 | I-759 | I-779 | I-805 |
| I-807 | I-810 | I-813 | I-815 | I-687 |
| I-880 | I-881 | I-884 | I-885 | I-886 |
| I-888 | I-889 | I-890 | I-892 | I-893 |
| I-894 | I-895 | I-896 | I-899 | I-926 |
| I-928 | I-930 | I-933 | I-969 | I-970 |
| I-971 | I-972 | I-991 | I-994 | I-995 |
| I-1006 | I-1008 | I-1016 | I-1112 | I-1113 |
| I-1114 | I-1199 | I-1678 | I-1802 | I-1803 |
| I-1804 | I-1805 | I-1806 | I-1807 | I-1808 |
| I-1809 | I-1810 | I-1811 | I-1812 | I-1813 |
| I-1818 | I-1819 | I-1820 | I-1821 | I-1822 |
| I-1823 | I-1824 | I-1825 | I-1826 | I-1830 |
| I-1831 | I-1832 | I-1894 | I-1915 | I-1919 |
| I-1920 | I-1933 | I-1958 | I-1960 | I-2014 |
| I-2017 | I-2038 | I-2039 | I-2093 | I-2415 |
| I-2416 | I-2756 | I-2764 | I-2765 | I-2776 |
| I-3197 | I-3271 | I-3272 | I-3275 | I-3278 |
| I-3397 | I-3681 | I-3708 | | |

A mixture of (S)—N-((3-chloro-2,6-difluorophenyl)(cyclopentyl)methyl)-1-((2-(trimethylsilyl)ethoxy)methyl)-1H-benzo[d]imidazol-2-amine (150 mg, 305 μmol) in TFA (2 mL) was stirred for 1 hour at 25° C. The reaction mixture was concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 40% B to 70% B in 8 min, then 70% B; wavelength: 220 nm; RT (min): 7.83). Concentration in vacuo gave (S)—N-((3-chloro-2,6-difluorophenyl)(cyclopentyl)methyl)-1H-benzo[d]imidazol-2-amine (20 mg, 55 μmol) as an off-white amorphous solid. ¹H NMR (400 MHz, DMSO-d₆) δ 10.31 (s, 1H), 7.53 (td, J=8.6, 5.4 Hz, 1H), 7.20-7.09 (m, 3H), 7.07 (d, J=8.9 Hz, 1H), 6.84 (s, 2H), 5.13 (t, J=9.7 Hz, 1H), 2.51 (s, 1H), 1.96-1.88 (m, 1H), 1.64 (s, 2H), 1.57 (dt, J=15.2, 8.1 Hz, 2H), 1.44 (td, J=12.5, 6.6 Hz, 2H), 1.17-1.09 (m, 1H). LCMS RT 0.815 min, [M+H]⁺ 362.05, LCMS method C.

### Example 38

### (1RS,3RS)—N—((S)-(3-chloro-2,6-difluorophenyl)(cyclopentyl)methyl)-3-(1H-imidazol-2-yl)cyclopentane-1-carboxamide and (1RS,3SR)—N—((S)-(3-chloro-2,6-difluorophenyl)(cyclopentyl)methyl)-3-(1H-imidazol-2-yl)cyclopentane-1-carboxamide

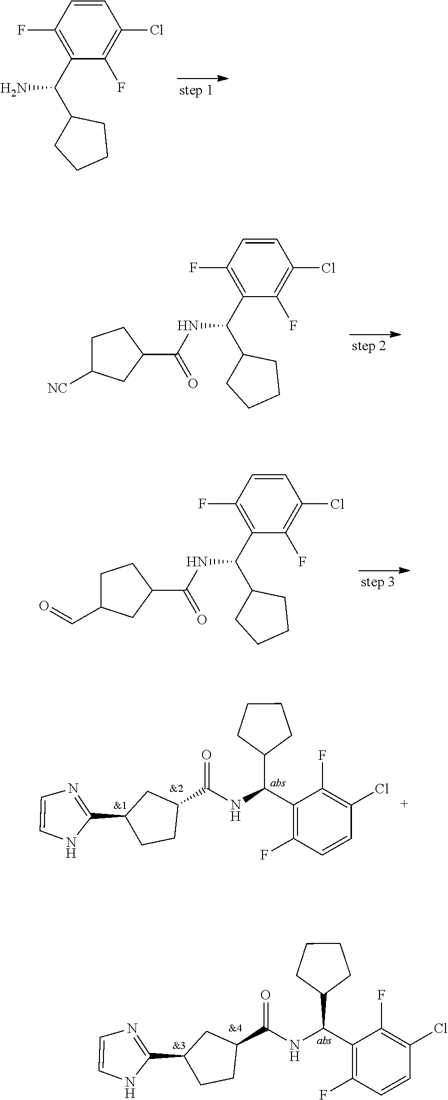 [SMILES: N[C@H](c1c(F)ccc(Cl)c1F)C1CCCC1 ; N#CC1CCC(C(=O)N[C@H](c2c(F)ccc(Cl)c2F)C2CCCC2)C1 ; O=CC1CCC(C(=O)N[C@H](c2c(F)ccc(Cl)c2F)C2CCCC2)C1 ; O=C(N[C@H](c1c(F)ccc(Cl)c1F)C1CCCC1)[C@@H]1CC[C@@H](c2ncc[nH]2)C1 ; O=C(N[C@H](c1c(F)ccc(Cl)c1F)C1CCCC1)[C@H]1CC[C@@H](c2ncc[nH]2)C1]

### Step 1. Synthesis of N—((S)-(3-chloro-2,6-difluorophenyl)(cyclopentyl)methyl)-3-cyanocyclopentane-1-carboxamide

A flask equipped with a magnetic stirrer bar was charged with 3-cyanocyclopentane-1-carboxylic acid (160 mg, 1.15 mmol) and (S)-(3-chloro-2,6-difluorophenyl)(cyclopentyl)methanamine (324 mg, 1.15 mmol). DMF (2 mL) was added, followed by DIPEA (601 μL, 3.45 mmol) and T3P (1.10 g, 50% wt, 1.72 mmol) dropwise. The reaction mixture stirred at ambient temperature for 30 minutes. The reaction was diluted with EtOAc (10 mL) and H₂O (30 mL). The organic layer was washed twice with water, then saturated NH₄Cl solution, and finally brine. The organic layer was dried over Na₂SO₄ and concentrated under reduced pressure. The crude material was purified over a reverse phase column chromatography (mobile phase A: 10 mM ammonium formate in water, mobile phase B: acetonitrile; gradient: A:B 90:10 to 30:70) to give N—((S)-(3-chloro-2,6-difluorophenyl)(cyclopentyl)methyl)-3-cyanocyclopentane-1-carboxamide (320 mg). LCMS RT 1.79 min, [M+H]⁺ 367.2, RT 1.82 min, [M+H]⁺ 367.2, LCMS method L.

### Step 2. Synthesis of N—((S)-(3-chloro-2,6-difluorophenyl)(cyclopentyl)methyl)-3-formylcyclopentane-1-carboxamide

A flame-dried microwave vial equipped with a magnetic stirrer bar was charged with N—((S)-(3-chloro-2,6-difluorophenyl)(cyclopentyl)methyl)-3-cyanocyclopentane-1-carboxamide (100 mg, 273 μmol). DCM (2 mL) was added under a N₂ atmosphere and the reaction was cooled down to −78° C. Diisobutylaluminum hydride (654 μL, 1 M in DCM, 654 μmol) was added dropwise and the reaction was stirred for 40 mins at −78° C. The reaction was warmed up to room temperature, diluted with DCM and quenched with Rochelle salt solution (10 mL). The biphasic mixture was allowed to stir for 15 minutes and the aqueous layer was extracted with DCM twice. The organic layers were combined, dried over Na₂SO₄ and concentrated under reduced pressure to afford N—((S)-(3-chloro-2,6-difluorophenyl)(cyclopentyl)methyl)-3-formylcyclopentane-1-carboxamide as a colorless viscous oil (100 mg), which was used in the next step without purification. LCMS RT 1.81 min, [M+H]⁺ 370.2, LCMS method L.

### Step 3. Synthesis of (1RS,3RS)—N—((S)-(3-chloro-2,6-difluorophenyl)(cyclopentyl)methyl)-3-(1H-imidazol-2-yl)cyclopentane-1-carboxamide and (1RS,3SR)—N—((S)-(3-chloro-2,6-difluorophenyl)(cyclopentyl)methyl)-3-(1H-imidazol-2-yl)cyclopentane-1-carboxamide

To a solution of N—((S)-(3-chloro-2,6-difluorophenyl)(cyclopentyl)methyl)-3-formylcyclopentane-1-carboxamide (40.0 mg, 108 μmol) in ethanol (0.5 mL) at 0° C. was added a solution of glyoxal (18.6 μL, 40% wt. in water, 162 μmol) and NH₄OH (145 μL, 29% wt, 1.08 mmol). The reaction mixture was allowed to warm up to room temperature and stirred for 5 hours. The reaction mixture was diluted with EtOAc and water. The aqueous phase was extracted twice with EtOAc. The organic phases were combined, dried over Na₂SO₄ and concentrated in vacuo. The crude mixture was purified on a reverse phase column (30 g), eluent: 10 mM ammonium formate in water:acetonitrile 95:5 to 50:50 in 16 minutes to give the two racemates. Racemate 1: 5.3 mg; LCMS RT 2.79 min, [M+H]⁺ 408.3, LCMS method M.; ¹HNMR (400 MHz, DMSO-d6) δ 8.37 (d, J=7.4 Hz, 1H), 8.18 (s, 1H), 7.48 (app. td, J=8.5, 4.3 Hz, 1H), 7.08 (app. t, J=9.3 Hz, 1H), 6.80 (s, 1H), 6.79 (s, 1H), 4.78 (dd, J=10.8, 7.7 Hz, 1H), 3.18-3.05 (m, 1H), 2.89-2.79 (m, 1H), 2.42-2.34 (m, 1H), 2.03-1.62 (m, 6H), 1.62-1.36 (m, 5H), 1.35-1.16 (m, 2H), 1.01-0.87 (m, 1H). Racemate 2: 4.1 mg, 80:20 mixture of racemate 2: racemate 1. LCMS RT 2.97 min, [M+H]⁺ 408.3, LCMS method M. ¹HNMR (400 MHz, DMSO-d6) δ 8.87 (overlapping d, J=6.3 Hz, 1H), 8.86 (overlapping d, J=6.7 Hz, 1H), 8.28 (br. ss, 1H), 7.56-7.47 (m, 1H), 7.16-7.08 (m, 1H), 6.85 (overlapping br. s, 1H), 6.83 (overlapping br. s, 1H), 4.84 (br. dd, J=10.9, 7.6 Hz, 1H), 3.14 (overlapping m, 1H), 2.83-2.72 (m, 1H), 2.44 (overlapping m, 1H), 2.24-2.13 (m, 0.5H), 2.13-2.03 (m, 0.5H), 1.99-1.67 (m, 6H), 1.65-1.41 (m, 4H), 1.39-1.20 (m, 2H), 1.07-0.95 (m, 1H).

Additional compounds prepared according to the methods of Example 38 are listed in Table 6 below. Corresponding ¹H NMR and mass spectrometry characterization for these compounds are described in Table 1. Certain compounds in Table 6 below were prepared with other compounds whose preparation is described further below in the Examples.

TABLE 6
Additional exemplary compounds

| | |
|---|---|
| I-2055 | I-2058 |

### Example 39

### (1r,3S)—N—((S)-(2,3-dichloro-6-fluoro-5-hydroxyphenyl)(4-fluorobicyclo[2.2.1]heptan-1-yl)methyl)-3-((pyridazin-3-ylmethyl)amino)cyclobutane-1-carboxamide

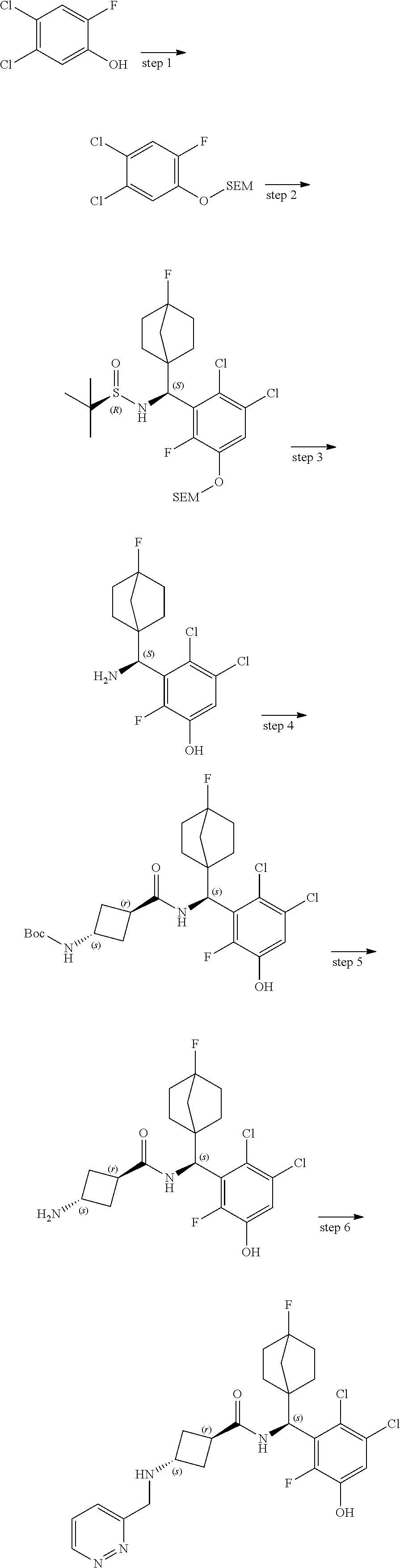

### Step 1. Synthesis of (2-((4,5-dichloro-2-fluorophenoxy)methoxy)ethyl)trimethylsilane

To a mixture of 4,5-dichloro-2-fluorophenol (7.5 g, 41.4 mmol) and K₂CO₃ (11.45 g, 82.9 mmol) in acetonitrile (75 mL) was added SEM-Cl (11.0 mL, 62.2 mmol) dropwise at 0° C. under a nitrogen atmosphere. The mixture was stirred for 1 hour at 25° C. The reaction was quenched with water (100 mL) and extracted with ethyl acetate (200 mL) three times. The combined organic layers were washed with brine, dried over sodium sulfate, filtered and concentrated in vacuo. The residue was purified by silica gel chromatography (30 g column; eluting with petroleum ether) to afford (2-((4,5-dichloro-2-fluorophenoxy)methoxy)ethyl)trimethylsilane (12 g, 39 mmol) as a colorless oil. ¹H NMR (400 MHz, DMSO-d₆) δ 7.77 (d, J=10.8 Hz, 1H), 7.58 (d, J=8.1 Hz, 1H), 5.39 (s, 2H), 3.81-3.72 (m, 2H), 0.96-0.83 (m, 2H), 0.03-0.01 (m, 9H).

### Step 2. Synthesis of (R)—N—((S)-(2,3-dichloro-6-fluoro-5-((2-(trimethylsilyl)ethoxy) methoxy)phenyl)(4-fluorobicyclo[2.2.1]heptan-1-yl)methyl)-2-methylpropane-2-sulfinamide

To a mixture of (2-((4,5-dichloro-2-fluorophenoxy)methoxy)ethyl)trimethylsilane (666 mg, 2.14 mmol) in THF (15 mL) was added LDA (1.53 mL, 2 M in THF, 3.06 mmol) dropwise at −60° C. under a nitrogen atmosphere. The mixture was stirred for 1 h at −60° C. prior to the addition of (R)—N-((4-fluorobicyclo[2.2.1]heptan-1-yl)methylene)-2-methylpropane-2-sulfinamide (500 mg, 2.04 mmol) at −60° C. The mixture was stirred for 1 h at room temperature. The reaction was quenched with saturated NH₄Cl (aq.). The reaction mixture was di luted with water (10 mL), and the aqueous phase was extracted with ethyl acetate (30 mL) three times. The combined organic layers were washed with brine, dried over sodium sulfate, filtered and concentrated in vacuo. The residue was purified by reverse phase flash chromatography (column: C18 silica gel; mobile phase A: water, mobile phase B: acetonitrile; gradient: 10% to 50% B in 10 min; detector: UV 254 nm) to give (R)—N—((S)-(2,3-dichloro-6-fluoro-5-((2-(trimethylsilyl)ethoxy)methoxy)phenyl)(4-fluorobicyclo[2.2.1]heptan-1-yl)methyl)-2-methylpropane-2-sulfinamide (990 mg, 1.78 mmol). LCMS RT 1.440 min, [M+H]⁺ 556.15, LCMS method C.

### Step 3. Synthesis of (S)-3-(amino(4-fluorobicyclo[2.2.1]heptan-1-yl)methyl)-4,5-dichloro-2-fluorophenol

A mixture of (R)—N—((S)-(2,3-dichloro-6-fluoro-5-((2-(trimethylsilyl)ethoxy)methox-y)phenyl) (4-fluorobicyclo[2.2.1]heptan-1-yl)methyl)-2-methylpropane-2-sulfinamide (990 mg, 1.78 mmol) in HCl (10 mL, 4 N in 1,4-dioxane) was stirred for 1 h at room temperature. The mixture was concentrated to afford (S)-3-(amino(4-fluorobicyclo[2.2.1]heptan-1-yl)me thyl)-4,5-dichloro-2-fluorophenol (543 mg, 1.69 mmol) as a yellow oil. LCMS RT 0.730 mi n, [M+H]⁺ 322.0, LCMS method C.

### Step 4. Synthesis of tert-butyl ((1S,3r)-3-(((S)-(2,3-dichloro-6-fluoro-5-hydroxyph-enyl)(4-fluorobicyclo[2.2.1]heptan-1-yl)methyl)carbamoyl)cyclobutyl)carbamate

To a mixture of (S)-3-(amino(4-fluorobicyclo[2.2.1]heptan-1-yl)methyl)-4,5-dichloro-2-fluorophenol (150 mg, 466 μmol), (1r,3r)-3-((tert-butoxycarbonyl)amino)cyclobutane-1-carboxylic acid (100 mg, 466 μmol) and NaHCO₃ (117 mg, 1.40 mmol) in DMF (2 mL) was added HATU (266 mg, 698 μmol). The mixture was stirred for 1 h at room temperature. The reaction mixture was diluted with water (30 mL), and the aqueous phase was extracted with ethyl acetate (50 mL) three times. The combined organic layers were washed with brine, dried over sodium sulfate, filtered and concentrated in vacuo. The resulting crude material was purified by C18 flash to afford tert-butyl ((1S,3r)-3-(((S)-(2,3-dichloro-6-fluoro-5-hydrox-yphenyl)(4-fluorobicyclo[2.2.1]heptan-1-yl)methyl)carbamoyl)cyclobutyl)carbamate (178 mg, 343 μmol) as a colorless oil. LCMS RT 1.127 min, [M+H]⁺ 463, LCMS method C.

### Step 5. Synthesis of (1r,3S)-3-amino-N—((S)-(2,3-dichloro-6-fluoro-5-hydroxyphen-yl)(4-fluorobicyclo[2.2.1]heptan-1-yl)methyl)cyclobutane-1-carboxamide

A mixture of ((1S,3r)-3-(((S)-(2,3-dichloro-6-fluoro-5-hydroxyphenyl)(4-fluorobicyclo[2.2.1]heptan-1-yl)methyl)carbamoyl)cyclobutyl)carbamate (158 mg, 304 μmol) in HCl (3 mL, 4 N in dioxane) was stirred for 1 h at 25° C. The mixture was concentrated and the residue was diluted with saturated NaHCO₃ solution. The reaction mixture was extracted with ethyl acetate (50 mL) three times. The combined organic layers were washed with brine, dried over sodium sulfate, filtered and concentrated in vacuo to afford (1r,3S)-3-amino-N—((S)-(2,3-dichloro-6-fluoro-5-hydroxyphenyl)(4-fluorobicyclo[2.2.1]heptan-1-yl)methyl)cyclobut-ane-1-carboxamide (90 mg, 0.21 mmol) as an off-white solid. LCMS RT 0.431 min, [M+H]⁺419. LCMS method C.

### Step 6. Synthesis of (1r,3S)—N—((S)-(2,3-dichloro-6-fluoro-5-hydroxyphenyl)(4-flu orobicyclo[2.2.1]heptan-1-yl)methyl)-3-((pyridazin-3-ylmethyl)amino)cyclobutane-1-carboxamide

A mixture of pyridazine-3-carbaldehyde (7.7 mg, 72 μmol) and (1r,3S)-3-amino-N—((S)-(2,3-dichloro-6-fluoro-5-hydroxyphenyl)(4-fluorobicyclo[2.2.1]heptan-1-yl)methyl)cycl-obutane-1-carboxamide (30 mg, 72 μmol) in MeOH (1 mL) was stirred for 0.5 h at 2° C. prio r to the addition of NaBH₃CN (5.4 mg, 85 μmol). The mixture was stirred for 1 h at 25° C. The mixture was diluted with water (20 mL) and extracted with ethyl acetate (50 mL) three ti mes. The combined organic layers were washed with brine, dried over sodium sulfate, filtere d and concentrated in vacuo. The resulting crude material was purified by preparative HPLC (column: XBridge Shield RP18 OBD Column, 30*150 mm, 5 μm; mobile phase A: water (10 mM NH₄HCO₃+0.1% NH₄OH), mobile phase B: MeOH; flow rate: 60 mL/min; gradient: 38% B to 56% B in 11 min; wavelength: 220/254 nm; RT (min): 10.6; injection volume: 0.475 mL) to give (1r,3S)—N—((S)-(2,3-dichloro-6-fluoro-5-hydroxyphenyl)(4-fluorobicyclo[2.2.1]heptan-1-yl) methyl)-3-((pyridazin-3-ylmethyl)amino)cyclobutane-1-carboxamide (16 m g, 31 μmol) as an off-white amorphous solid. ¹H NMR (400 MHz, DMSO-d₆) δ 9.11 (dd, J=4.7, 1.9 Hz, 1H), 8.04 (d, J=8.4 Hz, 1H), 7.70 (dd, J=8.5, 1.9 Hz, 1H), 7.65 (dd, J=8.5, 4.7 Hz, 1H), 7.10 (d, J=8.1 Hz, 1H), 6.53 (s, 1H), 6.29 (s, 1H), 5.51-5.33 (m, 1H), 3.93 (s, 2H), 3.32 (t, J=7.4 Hz, 1H), 3.11-3.02 (m, 1H), 2.18 (dq, J=7.8, 4.1, 3.2 Hz, 1H), 2.10-1.88 (m, 3H), 1.81-1.52 (dd, J=22.7, 10.1 Hz, 10H). LCMS RT 0.872 min, [M+H]⁺ 511.15, LCMS method B.

Additional compounds prepared according to the methods of Example 39 are listed in Table 7 below. Corresponding ¹H NMR and mass spectrometry characterization for these compounds are described in Table 1. Certain compounds in Table 7 below were prepared with other compounds whose preparation is described further below in the Examples.

TABLE 7
Additional exemplary compounds

| | | | |
|---|---|---|---|
| I-2992 | I-2993 | I-2994 | I-2995 |

### Example 40

### (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide

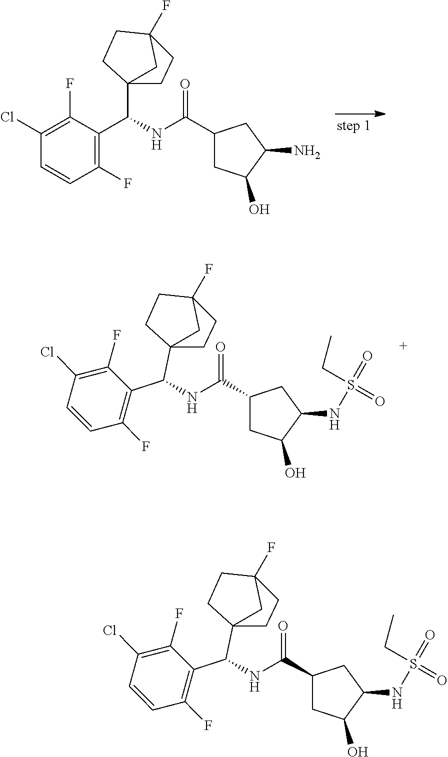 [SMILES: N[C@@H]1CC(C(=O)N[C@H](c2c(F)ccc(Cl)c2F)C23CCC(F)(CC2)C3)C[C@@H]1O ; CCS(=O)(=O)N[C@@H]1C[C@@H](C(=O)N[C@H](c2c(F)ccc(Cl)c2F)C23CCC(F)(CC2)C3)C[C@@H]1O ; CCS(=O)(=O)N[C@@H]1C[C@H](C(=O)N[C@H](c2c(F)ccc(Cl)c2F)C23CCC(F)(CC2)C3)C[C@@H]1O]

### Step 1. Synthesis of (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide

To a mixture of (3R,4S)-3-amino-N—((S)-(3-chloro-2,6-difluorophenyl)(4-fluorobicyclo[2.2.1]heptan-1-yl)methyl)-4-hydroxycyclopentane-1-carboxamide (50 mg, 0.12 mmol) and DIEA (63 μL, 0.36 mmol) in DCM (2 mL) was added ethanesulfonyl chloride (19 mg, 0.14 mmol) dropwise at 0° C. The mixture was stirred for 1 h at 25° C. The mixture was concentrated in vacuum. The resulting crude material was purified by preparative HPLC (column: XBridge Prep OBD C18 Column, 30*150 mm, 5 μm; mobile phase A: water (10 mM NH₄HCO₃+0.05% NH₄OH), mobile phase B: acetonitrile; flow rate: 60 mL/min; gradient: 38% B to 52% B in 7 min; wavelength: 254/220 nm nm; RT (min): 7.45) to afford (3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide (40 mg, 65 μmol) as a white amorphous solid. LCMS RT 1.098 min, [M+H]⁺ 509.05, LCMS method B.

The product was further purified by preparative chiral HPLC (column: CHIRALPAK ID, 2*25 cm, 5 μm; mobile phase A: hexane (0.5% 2 M NH₃ in MeOH), mobile phase B: EtOH:DCM 1:1; flow rate: 20 mL/min; gradient: 30% isocratic; wavelength: 220/254 nm; RT1 (min): 6.94; RT2 (min): 11.38; sample solvent: EtOH:DCM 1:1; injection volume: 1.9 mL) to afford (1R,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide and (1S,3R,4S)—N—((S)-(3-chloro-2,6-difluorophenyl)(4-fluorobicyclo[2.2.1]heptan-1-yl)methyl)-3-(ethylsulfonamido)-4-hydroxycyclopentane-1-carboxamide, both as a white amorphous solid.

Isomer 1: 4 mg, 8 μmol. LCMS RT 1.094 min, [M+H]⁺ 509.10, LCMS method B.

Isomer 2: 5.2 mg, 10 μmol. LCMS RT 1.092 min, [M+H]⁺ 509.10, LCMS method B.

### Example 41

### (S)—N-((3-chloro-2,6-difluorophenyl)(cyclopentyl)methyl)-1-phenylmethanesulfonamide

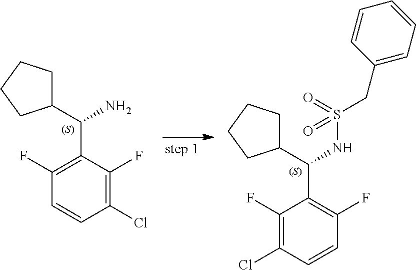

### Step 1. Synthesis of (S)—N-((3-chloro-2,6-difluorophenyl) (cyclopentyl)methyl)-1-phenylmethanesulfonamide

To a mixture of (S)-(3-chloro-2,6-difluorophenyl) (cyclopentyl)methanamine (100 mg, 407 μmol) and TEA (206 mg, 2.04 mmol) in DCM (1 mL) was added phenylmethanesulfonyl chloride (93.1 mg, 488 μmol) at room temperature. The mixture was stirred for 1 hour at room temperature. The reaction was quenched with MeOH (1 ml) and concentrated. The residue was first purified by reverse phase flash chromatography (column: C18 silica gel; mobile phase A: water, mobile phase B: acetonitrile; gradient: 10% to 60% B in 10 min; detector: UV 220 nm), then purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 50% B to 80% B in 7 min, then 80% B; wavelength: 220 nm; RT1 (min): 6.6) to give (S)—N-((3-chloro-2,6-difluorophenyl) (cyclopentyl)methyl)-1-phenylmethanesulfonamide (32.1 mg, 80 mol) as an off-white amorphous solid. ¹H NMR (400 MHz, DMSO-d6) δ 7.91 (d, J=7.8 Hz, 1H), 7.57 (td, J=8.7, 5.5 Hz, 1H), 7.30-7.21 (m, 3H), 7.19-7.10 (m, 3H), 4.42 (dd, J=10.7, 7.7 Hz, 1H), 4.26-4.07 (m, 2H), 2.39 (p, J=8.6 Hz, 1H), 1.92 (dp, J=12.3, 6.8, 5.8 Hz, 1H), 1.69-1.34 (m, 5H), 1.32-1.19 (m, 1H), 0.92 (dq, J=12.2, 8.0 Hz, 1H). LCMS RT 1.692 min [M+Na]⁺ 422, LCMS method C.

Additional compounds prepared according to the methods of Examples 40-41 are listed in Table 8 below. Corresponding ¹H NMR and mass spectrometry characterization for these compounds are described in Table 1. Certain compounds in Table 8 below were prepared with other compounds whose preparation is described further below in the Examples.

TABLE 8
Additional exemplary compounds

| | | | | |
|---|---|---|---|---|
| I-1816 | I-1827 | I-1959 | I-2009 | I-2151 |
| I-2152 | I-2153 | I-2681 | I-2683 | I-2694 |
| I-2695 | I-2701 | I-2702 | I-2703 | I-2704 |
| I-2705 | I-2706 | I-2707 | I-2748 | I-2749 |
| I-2750 | I-2751 | I-2766 | I-2767 | I-2768 |
| I-2769 | I-2819 | I-2820 | I-2821 | I-2822 |
| I-2847 | I-2848 | I-2849 | I-2850 | I-2851 |
| I-2852 | I-2853 | I-2854 | I-2855 | I-2856 |
| I-2882 | I-2883 | I-2896 | I-2897 | I-2898 |
| I-2899 | I-2900 | I-2901 | I-2907 | I-2908 |
| I-2909 | I-2910 | I-2911 | I-2912 | I-2913 |
| I-2914 | I-2930 | I-2931 | I-2932 | I-2933 |
| I-2934 | I-2935 | I-2936 | I-2937 | I-2938 |
| I-2939 | I-2940 | I-2941 | I-2968 | I-2969 |
| I-2970 | I-2971 | I-2972 | I-2986 | I-2987 |
| I-2988 | I-3030 | I-3031 | I-3032 | I-3033 |
| I-3034 | I-3035 | I-3036 | I-3037 | I-3038 |
| I-3039 | I-3040 | I-3041 | I-3042 | I-3043 |
| I-3066 | I-3072 | I-3074 | I-3075 | I-3076 |
| I-3077 | I-3078 | I-3079 | I-3080 | I-3090 |
| I-3097 | I-3107 | I-3108 | I-3114 | I-3116 |
| I-3117 | I-3133 | I-3135 | I-3136 | I-3142 |
| I-3143 | I-3155 | I-3158 | I-3159 | I-3162 |
| I-3163 | I-3165 | I-3199 | I-3200 | I-3201 |
| I-3205 | I-3206 | I-3207 | I-3208 | I-3209 |
| I-3210 | I-3211 | I-3229 | I-3230 | I-3231 |
| I-3236 | I-3237 | I-3295 | I-3296 | I-3299 |
| I-3300 | I-3301 | I-3302 | I-3303 | I-3304 |
| I-3305 | I-3306 | I-3307 | I-3308 | I-3309 |
| I-3310 | I-3311 | I-3312 | I-3313 | I-3314 |
| I-3315 | I-3322 | I-3323 | I-3330 | I-3343 |
| I-3344 | I-3345 | I-3346 | I-3347 | I-3348 |
| I-3349 | I-3360 | I-3361 | I-3362 | I-3363 |
| I-3364 | I-3365 | I-3366 | I-3368 | I-3369 |
| I-3370 | I-3371 | I-3372 | I-3399 | I-3400 |
| I-3407 | I-3412 | I-3413 | I-3418 | I-3419 |
| I-3420 | I-3421 | I-3422 | I-3424 | I-3445 |
| I-3446 | I-3447 | I-3448 | I-3452 | I-3453 |
| I-3454 | I-3455 | I-3456 | I-3457 | I-3458 |
| I-3459 | I-3460 | I-3461 | I-3462 | I-3463 |
| I-3464 | I-3465 | I-3466 | I-3467 | I-3468 |
| I-3469 | I-3470 | I-3471 | I-3472 | I-3473 |
| I-3474 | I-3475 | I-3476 | I-3477 | I-3478 |
| I-3480 | I-3481 | I-3482 | I-3483 | I-3484 |
| I-3485 | I-3486 | I-3517 | I-3520 | I-3523 |
| I-3524 | I-3525 | I-3549 | I-3550 | I-3551 |
| I-3552 | I-3553 | I-3554 | I-3555 | I-3556 |
| I-3557 | I-3558 | I-3559 | I-3560 | I-3561 |
| I-3562 | I-3563 | I-3564 | I-3565 | I-3566 |
| I-3567 | I-3568 | I-3569 | I-3570 | I-3571 |
| I-3572 | I-3573 | I-3574 | I-3575 | I-3576 |
| I-3577 | I-3578 | I-3579 | I-3580 | I-3581 |
| I-3582 | I-3583 | I-3584 | I-3585 | I-3594 |
| I-3601 | I-3602 | I-3603 | I-3604 | I-3611 |
| I-3612 | I-3613 | I-3614 | I-3631 | I-3632 |
| I-3633 | I-3651 | I-3652 | I-3653 | I-3654 |
| I-3655 | I-3656 | I-3657 | I-3658 | I-3659 |
| I-3660 | I-3661 | I-3662 | I-3663 | I-3664 |
| I-3665 | I-3666 | I-3667 | I-3668 | I-3669 |
| I-3670 | I-3672 | I-3684 | I-3685 | I-3686 |
| I-3687 | I-3714 | I-3715 | I-3716 | I-3741 |
| I-3742 | I-3743 | I-3744 | I-3745 | |

### Example 42

### N-(2-(3,4-dichlorophenyl)-2-methylpropyl)quinolin-2-amine

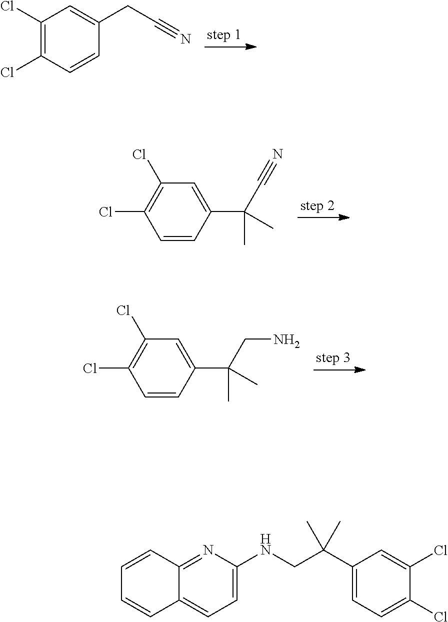 [SMILES: N#CCc1ccc(Cl)c(Cl)c1 ; CC(C)(C#N)c1ccc(Cl)c(Cl)c1 ; CC(C)(CN)c1ccc(Cl)c(Cl)c1 ; CC(C)(CNc1ccc2ccccc2n1)c1ccc(Cl)c(Cl)c1]

### Step 1. Synthesis of 2-(3,4-dichlorophenyl)-2-methylpropanenitrile

To a mixture of 2-(3,4-dichlorophenyl)acetonitrile (1000 mg, 5.38 mmol) in THF (15 mL) was added LiHMDS (13.4 mL, 1 M in THF, 13.4 mmol) dropwise at 0° C. under a nitrogen atmosphere. The mixture was stirred for 1 h at 0° C. prior to the addition of Mel (1.91 g, 13.4 mmol). The mixture was stirred for 1 hour at 25° C. The reaction was quenched with saturated NH₄Cl (aq.). The mixture was diluted with water (20 mL), and the aqueous phase was extracted with ethyl acetate (50 mL) three times. The combined organic layers were washed with brine, dried over sodium sulfate, filtered and concentrated in vacuo. The residue was purified by preparative TLC (petroleum ether/ethyl acetate, ratio 5/1) to afford 2-(3,4-dichlorophenyl)-2-methylpropanenitrile (1.0 g, 4.67 mmol) as a yellow oil. ¹HNMR (400 MHz, DMSO-d6) 7.78 (d, J=2.4 Hz, 1H), 7.71 (d, J=8.5 Hz, 1H), 7.54 (dd, J=8.5, 2.3 Hz, 1H), 1.70 (s, 6H).

### Step 2. Synthesis of 2-(3,4-dichlorophenyl)-2-methylpropan-1-amine

To a mixture of 2-(3,4-dichlorophenyl)-2-methylpropanenitrile (1.0 g, 4.67 mmol) in THF (10 mL) was added LiAlH₄ (213 mg, 5.60 mmol) in portions at 0° C. under a nitrogen atmosphere. The mixture was stirred for 2 hours at 80° C. The reaction was then cooled to 0° C. and quenched with water (3 mL), sodium hydroxide (6 mL, 4 N in water) and water (3 m L). The reaction mixture was filtered through a pad of Celite, the pad was washed with ethyl acetate, and the filtrate was concentrated in vacuo to give 2-(3,4-dichlorophenyl)-2-methylp-ropan-1-amine (900 mg, 1.38 mmol) as a yellow oil. LCMS RT 0.526 min, [M+H]⁺ 218, LC-MS method C.

### Step 3. Synthesis of N-(2-(3,4-dichlorophenyl)-2-methylpropyl)quinolin-2-amine

A mixture of 2-chloroquinoline (200 mg, 1.22 mmol), 2-(3,4-dichlorophenyl)-2-methylpropan-1-amine (266 mg, 1.22 mmol), Pd₂(dba)₃ (111 mg, 122 μmol), BINAP (151 mg, 244 μmol) and t-BuONa (117 mg, 1.22 mmol) in toluene (4 mL) was stirred at 80° C. for 1 h. The mixture was diluted with water (20 ml) and extracted with ethyl acetate (20 ml*3). The combined organic layers were washed with brine, dried over Na₂SO₄ and concentrated. The residue was purified first by preparative TLC (MeOH:DCM 1:10) and then by preparative HPLC (column: XBridge Shield RP18 OBD Column, 30*150 mm, 5 μm; mobile phase A: water (10 mM NH₄HCO₃), mobile phase B: acetonitrile; flow rate: 60 mL/min; gradient: 55% B to 85% B in 7 min, then 85% B; wavelength: 254 nm; RT (min): 7.48) to give N-[2-(3,4-dichlorophenyl)-2-methylpropyl]quinolin-2-amine (91.3 mg, 264 μmol) as a colorless oil. ¹H NMR (400 MHz, DMSO-d6) δ 7.78 (d, J=8.9 Hz, 1H), 7.68 (d, J=2.2 Hz, 1H), 7.61-7.51 (m, 2H), 7.51-7.40 (m, 3H), 7.12 (ddd, J=8.0, 6.6, 1.6 Hz, 1H), 6.84-6.72 (m, 2H), 3.71 (d, J=5.8 Hz, 2H), 1.36 (s, 6H). LCMS RT 0.855 min, [M+H]⁺ 345.00, LCMS method C.

### Example 43

### 2-(3-(((1-(3-chlorophenyl)cyclobutyl)methyl)amino)-1H-pyrazol-1-yl)-N-methylacetamide

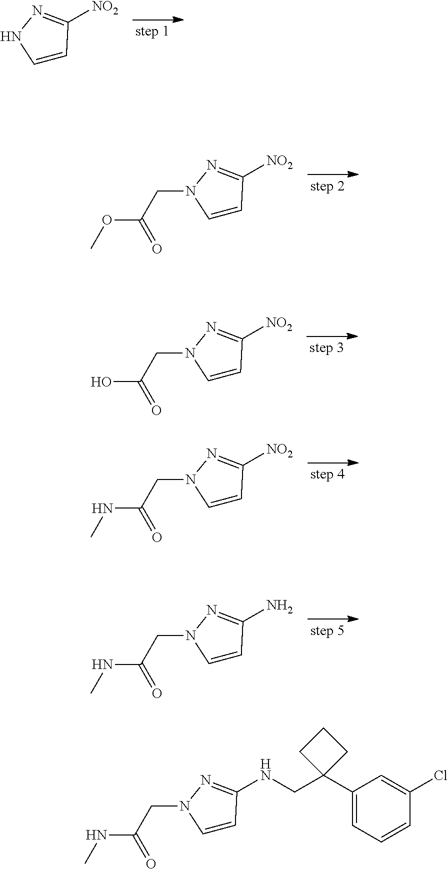 [SMILES: O=[N+]([O-])c1cc[nH]n1 ; COC(=O)Cn1ccc([N+](=O)[O-])n1 ; O=C(O)Cn1ccc([N+](=O)[O-])n1 ; CNC(=O)Cn1ccc([N+](=O)[O-])n1 ; CNC(=O)Cn1ccc(N)n1 ; CNC(=O)Cn1ccc(NCC2(c3cccc(Cl)c3)CCC2)n1]

### Step 1. Synthesis of methyl 2-(3-nitro-1H-pyrazol-1-yl)acetate

To a solution of 3-nitro-1H-pyrazole (5.00 g, 44 mmol) in DMF (30.0 mL) was added methyl 2-bromoacetate (4.18 mL, 44.2 mmol) and K₂CO₃ (12.2 g, 88.4 mmol). Then the mixture was stirred at 25° C. for 16 hours. The mixture was poured into water (30.0 mL) and extracted with ethyl acetate (30.0 mL*5). The combined organic layers were washed with brine (30.0 mL), dried over Na₂SO₄ and concentrated under vacuum. The residue was purified by column chromatography (SiO₂, petroleum ether/ethyl acetate 1/1) to give methyl 2-(3-nitro-1H-pyrazol-1-yl)acetate (6.28 g, 28.5 mmol) as a yellow oil. ¹H NMR (400 MHz, DMSO-d₆) δ 8.05 (d, J=2.6 Hz, 1H), 8.03-7.99 (m, 1H), 7.11-7.05 (m, 1H), 7.04-6.99 (m, 1H), 5.29 (s, 2H), 3.72 (s, 3H).

### Step 2. Synthesis of 2-(3-nitro-1H-pyrazol-1-yl)acetic acid

To a solution of methyl 2-(3-nitro-1H-pyrazol-1-yl)acetate (4.00 g, 18.1 mmol) in THF (40.0 mL) and H₂O (8.00 mL) was added LiOH·H₂O (3.81 g, 90.8 mmol). The mixture was stirred at 60° C. for 16 hours. Ethyl acetate (10.0 mL) and water (10.0 mL) were added and the layers were separated. The pH of the aqueous phase was adjusted to 2 with 1 N HCl, and the mixture was extracted with ethyl acetate (10.0 mL*3). Combined extracts were washed with brine (10.0 mL) and dried over Na₂SO₄. The mixture was filtered and concentrated under vacuum to give 2-(3-nitro-1H-pyrazol-1-yl)acetic acid (2.45 g, 14.3 mmol) as a yellow solid. ¹H NMR: (400 MHz, DMSO-d₆) δ 14.04-13.85 (m, 1H), 8.03 (s, 1H), 7.07 (d, J=2.4 Hz, 1H), 5.15 (s, 2H).

### Step 3. Synthesis of N-methyl-2-(3-nitro-1H-pyrazol-1-yl)acetamide

To a solution of 2-(3-nitro-1H-pyrazol-1-yl)acetic acid (2.00 g, 11.7 mmol) in DMF (20.0 mL) was added methanamine HCl salt (1.58 g, 23.3 mmol), HATU (5.78 g, 15.1 mmol) and DIEA (10.1 mL, 58.4 mmol). The mixture was stirred at 25° C. for 16 hours. The combined mixture was poured into water (20.0 mL) and extracted with ethyl acetate (20.0 mL*3). The combined organic layers were washed with brine (20.0 mL*3), dried over Na₂SO₄ and concentrated under vacuum. The residue was purified by silica gel column chromatography (petroleum ether/ethyl acetate 1/1) to give N-methyl-2-(3-nitro-1H-pyrazol-1-yl)acetamide (900 mg, 4.89 mmol) as a yellow oil. ¹H NMR: (400 MHz, DMSO-d₆) δ 8.21-8.13 (m, 1H), 7.99 (d, J=2.4 Hz, 1H), 7.05 (d, J=2.4 Hz, 1H), 4.95-4.93 (m, 2H), 2.64 (d, J=4.4 Hz, 3H).

### Step 4. Synthesis of 2-(3-amino-1H-pyrazol-1-yl)-N-methylacetamide

To a solution of N-methyl-2-(3-nitro-1H-pyrazol-1-yl)acetamide (800 mg, 4.34 mmol) in MeOH (10.0 mL) was added Pd/C (0.10 g, 10%) under a N₂ atmosphere. The suspension was degassed and purged with H₂ three times. The mixture was stirred under H₂ (15 psi) at 25° C. for 3 hours. The mixture was filtered and the filtrate was concentrated to give 2-(3-amino-1H-pyrazol-1-yl)-N-methylacetamide (544 mg, 3.53 mmol) as a colorless oil. ¹H NMR: (400 MHz, DMSO-d₆) δ 7.71 (br s, 1H), 7.30 (d, J=2.4 Hz, 1H), 5.40 (d, J=2.0 Hz, 1H), 4.42 (s, 2H), 2.59 (d, J=4.4 Hz, 3H).

### Step 5. Synthesis of 2-(3-(((1-(3-chlorophenyl)cyclobutyl)methyl)amino)-1H-pyrazol-1-yl)-N-methylacetamide

To a solution of 2-(3-amino-1H-pyrazol-1-yl)-N-methylacetamide (540 mg, 3.50 mmol) in MeOH (5.0 mL) was added 1-(3-chlorophenyl)cyclobutane-1-carbaldehyde (681 mg, 3.50 mmol). The mixture was stirred at 20° C. for 1 hour. NaBH₃CN (1.10 g, 17 mmol) was added at 0° C., and the mixture was stirred at 20° C. for 15 hours. The mixture was concentrated and the residue was purified by preparative HPLC (column: waters Xbridge 150*25 mm, 5 μm; mobile phase A: water (0.05% ammonia hydroxide v/v), mobile phase B: acetonitrile; gradient: 30%-60% B over 9 min) to give 2-(3-(((1-(3-chlorophenyl)cyclobutyl)methyl)amino)-1H-pyrazol-1-yl)-N-methylacetamide (281 mg, 838 umol) as a white solid. ¹H NMR: (400 MHz, DMSO-d₆)

δ 7.66 (br d, J=4.4 Hz, 1H), 7.36-7.28 (m, 2H), 7.24-7.17 (m, 2H), 7.17-7.12 (m, 1H), 5.37 (d, J=2.4 Hz, 1H), 4.86 (t, J=6.4 Hz, 1H), 4.42 (s, 2H), 3.31 (d, J=6.4 Hz, 2H), 2.58 (d, J=4.8 Hz, 3H), 2.31-2.14 (m, 4H), 2.10-1.97 (m, 1H), 1.84-1.71 (m, 1H).

### Example 44

### 6-(((1-(3,4-dichlorophenyl)cyclobutyl)methyl)amino)-N-methylpyridazine-3-carboxamide

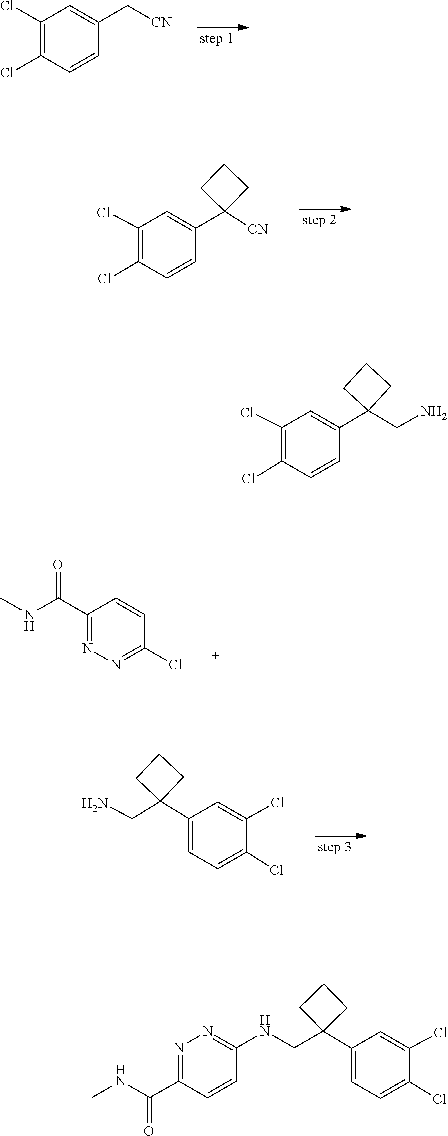 [SMILES: N#CCc1ccc(Cl)c(Cl)c1 ; N#CC1(c2ccc(Cl)c(Cl)c2)CCC1 ; NCC1(c2ccc(Cl)c(Cl)c2)CCC1 ; CNC(=O)c1ccc(Cl)nn1 ; NCC1(c2ccc(Cl)c(Cl)c2)CCC1 ; CNC(=O)c1ccc(NCC2(c3ccc(Cl)c(Cl)c3)CCC2)nn1]

### Step 1. Synthesis of 1-(3,4-dichlorophenyl)cyclobutane-1-carbonitrile

To a suspension of NaH (60% in mineral oil, 26.9 g, 672 mmol) in THF (100 mL) was added a solution of 2-(3,4-dichlorophenyl)acetonitrile (50.0 g, 269 mmol) in THF (200 mL) dropwise at 0° C. The mixture was stirred at 0° C. for 1 hour. Then 1,3-dibromopropane (57.0 g, 282 mmol) was added dropwise over 1.5 hours at 0° C. The mixture was warmed to 25° C. and stirred at 25° C. for 0.5 hr. The mixture was poured into saturated NH₄Cl solution (400 mL) and filtered. The filtrate was extracted with ethyl acetate (250 mL*3). The organic layers were washed with brine (250 mL), dried over Na₂SO₄, filtered and concentrated. The crude product was purified by silica gel column chromatography (petroleum ether:ethyl acetate 50:1 to 6:1) to give 1-(3,4-dichlorophenyl)cyclobutane-1-carbonitrile (22.1 g, 96.0 mmol) as a colorless oil. ¹H NMR: (400 MHz, CDCl₃) δ 7.55 (d, J=2.0 Hz, 1H), 7.52 (d, J=8.0 Hz, 1H), 7.32-7.29 (m, 1H), 2.90-2.85 (m, 2H), 2.64-2.62 (m, 2H), 2.61-2.47 (m, 1H), 2.15-2.09 (m, 1H).

### Step 2. Synthesis of (1-(3,4-dichlorophenyl)cyclobutyl)methanamine

To a suspension of LiAlH₄ (4.36 g, 115 mmol) in THF (100 mL) was added a solution of 1-(3,4-dichlorophenyl)cyclobutane-1-carbonitrile (20.0 g, 88.5 mmol) in THF (50.0 mL) dropwise at 0° C. The mixture was warmed to 25° C. and stirred at 25° C. for 1 hour. The stirring mixture was cooled to 10° C. Water (5.00 mL) was added, followed by 15% NaOH solution (5.00 mL), water (15.0 mL), and Na₂SO₄ (6.0 g). The mixture was filtered through celite. The filtrate was extracted with ethyl acetate (50.0 mL*2). The combined organic layers were washed with brine, dried over Na₂SO₄, filtered and concentrated to give (1-(3,4-dichlorophenyl)cyclobutyl)methanamine (11.0 g, 46.3 mmol) as a yellow oil. ¹H NMR: (400 MHz, CDCl₃) δ 7.28 (d, J=8.4 Hz, 1H), 7.19 (d, J=2.0 Hz, 1H), 6.96-6.94 (m, 1H), 2.93 (s, 2H), 2.31-2.26 (m, 2H), 2.16-2.11 (m, 2H), 2.09-2.02 (m, 1H), 1.92-1.84 (m, 1H).

### Step 3. Synthesis of 6-(((1-(3,4-dichlorophenyl)cyclobutyl)methyl)amino)-N-methylpyridazine-3-carboxamide

In a vial 6-chloro-N-methylpyridazine-3-carboxamide (25 mg, 0.15 mmol) and (1-(3,4-dichlorophenyl)cyclobutyl)methanamine (34 mg, 0.15 mmol) were dissolved in NMP (0.5 mL). DIEA (38 μL, 0.22 mmol) was added. The vial was sealed and heated at 100° C. over the weekend. After cooling to room temperature, the reaction was purified on AccQprep using 35-65% of acetonitrile (0.1% formic acid) in water to give 6-(((1-(3,4-dichlorophenyl)cyclobutyl)methyl)amino)-N-methylpyridazine-3-carboxamide (22 mg, 60 μmol). LCMS: RT 1.426 min, [M+H]⁺ 365.25. LCMS method K.

Additional compounds prepared according to the methods of Examples 42-44 are listed in Table 9 below. Corresponding ¹H NMR and mass spectrometry characterization for these compounds are described in Table 1. Certain compounds in Table 9 below were prepared with other compounds whose preparation is described further below in the Examples.

TABLE 9
Additional Exemplary Compounds that can
be synthesized similarly using
Buchwald, reductive amination, urea formation,
or amide coupling reactions

| | | | | |
|---|---|---|---|---|
| I-3 | I-6 | I-180 | I-181 | I-191 |
| I-192 | I-193 | I-194 | I-198 | I-199 |
| I-200 | I-201 | I-202 | I-203 | I-209 |
| I-210 | I-211 | I-212 | I-213 | I-214 |
| I-216 | I-217 | I-218 | I-222 | I-358 |
| I-360 | I-362 | I-364 | I-366 | I-367 |
| I-376 | I-377 | I-411 | I-429 | I-430 |
| I-437 | I-438 | I-439 | I-440 | I-441 |
| I-442 | I-470 | I-471 | I-475 | I-476 |
| I-477 | I-478 | I-488 | I-489 | I-490 |
| I-492 | I-504 | I-505 | I-506 | I-508 |
| I-509 | I-511 | I-512 | I-513 | I-514 |
| I-524 | I-526 | I-527 | I-528 | I-536 |
| I-537 | I-538 | I-539 | I-540 | I-541 |
| I-542 | I-543 | I-544 | I-545 | I-546 |
| I-547 | I-549 | I-550 | I-551 | I-559 |
| I-560 | I-561 | I-562 | I-569 | I-570 |
| I-576 | I-598 | I-600 | I-616 | I-619 |
| I-620 | I-622 | I-640 | I-641 | I-642 |
| I-643 | I-644 | I-659 | I-660 | I-661 |
| I-665 | I-678 | I-679 | I-680 | I-681 |
| I-682 | I-683 | I-684 | I-685 | I-686 |
| I-688 | I-744 | I-747 | I-755 | I-756 |
| I-758 | I-760 | I-761 | I-766 | I-771 |
| I-773 | I-778 | I-780 | I-781 | I-782 |
| I-783 | I-784 | I-785 | I-786 | I-788 |
| I-789 | I-792 | I-809 | I-811 | I-812 |
| I-814 | I-816 | I-820 | I-826 | I-827 |
| I-828 | I-829 | I-831 | I-832 | I-833 |
| I-836 | I-839 | I-840 | I-845 | I-864 |
| I-865 | I-866 | I-867 | I-868 | I-869 |
| I-870 | I-871 | I-921 | I-922 | I-923 |
| I-924 | I-375 | I-939 | I-940 | I-947 |
| I-974 | I-975 | I-990 | I-1007 | I-1009 |
| I-1010 | I-1015 | I-1020 | I-1021 | I-1022 |
| I-1023 | I-1024 | I-1027 | I-1042 | I-1043 |
| I-1044 | I-1117 | I-1118 | I-1119 | I-1202 |
| I-1214 | I-1215 | I-1216 | I-1220 | I-1222 |
| I-1223 | I-1224 | I-1225 | I-1259 | I-1260 |
| I-1275 | I-1276 | I-1277 | I-1278 | I-1328 |
| I-1329 | I-1330 | I-1331 | I-1332 | I-1333 |
| I-1334 | I-1335 | I-1343 | I-1363 | I-1493 |
| I-1506 | I-1507 | I-1508 | I-1509 | I-1510 |
| I-1511 | I-1512 | I-1513 | I-1514 | I-1515 |
| I-1516 | I-1640 | I-1641 | I-1642 | I-1685 |
| I-2270 | I-2273 | I-3196 | I-3198 | |

### Example 45

### (S)—N-((3-chloro-2,6-difluorophenyl)(cyclopentyl)methyl)-1H-benzo[d]imidazol-2-amine

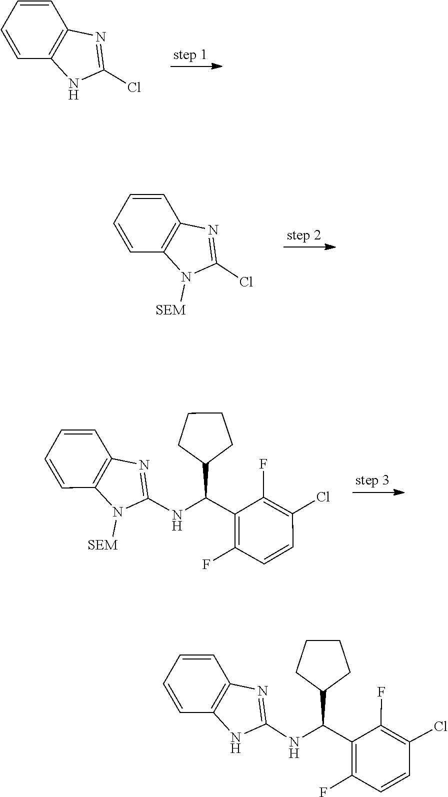 [SMILES: Clc1nc2ccccc2[nH]1 ; C[Si](C)(C)CCOCn1c(Cl)nc2ccccc21 ; C[Si](C)(C)CCOCn1c(N[C@H](c2c(F)ccc(Cl)c2F)C2CCCC2)nc2ccccc21 ; Fc1ccc(Cl)c(F)c1[C@@H](Nc1nc2ccccc2[nH]1)C1CCCC1]

### Step 1. Synthesis of 2-chloro-1-((2-(trimethylsilyl)ethoxy)methyl)-1H-benzo[d]imidazole

To a mixture of 2-chloro-1H-benzo[d]imidazole (800 mg, 5.24 mmol) and Cs₂CO₃ (5.12 g, 15.7 mmol) in DMF (5 mL) was added SEM-Cl (1.39 mL, 7.86 mmol) dropwise at 0° C. under a nitrogen atmosphere. The mixture was stirred for 1 hour at 25° C. The reaction mixture was diluted with water (30 mL), and the aqueous phase was extracted with ethyl acetate (50 mL) three times. The combined organic layers were washed with brine, dried over sodium sulfate, filtered and concentrated in vacuo. The resulting crude material was purified by C18 flash chromatography to afford 2-chloro-1-((2-(trimethylsilyl)ethoxy)methyl)-1H-benzo[d]imidazole (800 mg, 2.83 mmol) as an off-white solid. LCMS RT 0.987 min, [M+H]⁺ 283, LCMS method C.

### Step 2. Synthesis of (S)—N-((3-chloro-2,6-difluorophenyl)(cyclopentyl)methyl)-1-((2-(trimethylsilyl)ethoxy)methyl)-1H-benzo[d]imidazol-2-amine

A mixture of 2-chloro-1-((2-(trimethylsilyl)ethoxy)methyl)-1H-benzo[d]imidazole (300 mg, 1.06 mmol), (S)-(3-chloro-2,6-difluorophenyl)(cyclopentyl)methanamine (261 mg, 1.06 mmol), Cs₂CO₃ (1.04 g, 3.18 mmol), BINAP (66.0 mg, 106 μmol) and Pd₂(dba)₃ (110 mg, 106 μmol) in dioxane (3 mL) was stirred for 16 hours at 110° C. under a N₂ atmosphere. The reaction mixture was diluted with water (20 ml) and extracted with ethyl acetate (50 ml*3). The combined organic layers were washed with brine (10 ml), dried over sodium sulfat e, filtered and concentrated in vacuo. The resulting crude material was purified by C18 flash chromatography (CH₃CN/H₂O) to afford (S)—N-((3-chloro-2,6-difluorophenyl)(cyclopentyl)methyl)-1-((2-(trimethylsilyl)ethoxy)methyl)-1H-benzo[d]imidazol-2-amine (150 mg, 305 μmol) as a yellow oil. LCMS RT 1.604 min, [M+H]⁺ 492, LCMS method B.

### Step 3. Synthesis of (S)—N-((3-chloro-2,6-difluorophenyl)(cyclopentyl)methyl)-1H-benzo[d]imidazol-2-amine

A mixture of (S)—N-((3-chloro-2,6-difluorophenyl)(cyclopentyl)methyl)-1-((2-(trimet-hylsilyl)ethoxy)methyl)-1H-benzo[d]imidazol-2-amine (150 mg, 305 μmol) in TFA (2 mL) was stirred for 1 hour at 25° C. The reaction mixture was concentrated in vacuo. The resulting crude material was purified by preparative HPLC (column: XBridge Prep OBD C18 Column, 30*150 mm, 5 μm; mobile phase A: water (10 mM NH₄HCO₃), mobile phase B: acetonitrile; flow rate: 60 mL/min; gradient: 40% B to 70% B in 8 min, then 70% B; wavelength: 220 nm; RT (min): 7.83). Concentration in vacuo gave (S)—N-((3-chloro-2,6-difluorophenyl)(cyclopentyl)methyl)-1H-benzo[d]imidazol-2-amine (20 mg, 55 μmol) as an off-white amorphous solid. ¹H NMR (400 MHz, DMSO-d₆) δ 10.31 (s, 1H), 7.53 (td, J=8.6, 5.4 Hz, 1H), 7.20-7.09 (m, 3H), 7.07 (d, J=8.9 Hz, 1H), 6.84 (s, 2H), 5.13 (t, J=9.7 Hz, 1H), 2.51 (s, 1H), 1.96-1.88 (m, 1H), 1.64 (s, 2H), 1.57 (dt, J=15.2, 8.1 Hz, 2H), 1.44 (td, J=12.5, 6.6 Hz, 2H), 1.17-1.09 (m, 1H). LCMS RT 0.815 min, [M+H]⁺ 362.05, LCMS method C.

Additional compounds prepared according to the methods of Example 45 are listed in Table 10 below. Corresponding ¹H NMR and mass spectrometry characterization for these compounds are described in Table 1. Certain compounds in Table 10 below were prepared with other compounds whose preparation is described further below in the Examples.

TABLE 10
Additional exemplary compounds

| | | | | |
|---|---|---|---|---|
| I-183 | I-208 | I-224 | I-226 | I-227 |
| I-228 | I-229 | I-234 | I-236 | I-237 |
| I-238 | I-239 | I-240 | I-241 | I-242 |
| I-243 | I-244 | I-245 | I-246 | I-249 |
| I-250 | I-251 | I-269 | I-270 | I-271 |
| I-294 | I-295 | I-296 | I-373 | I-493 |
| I-515 | I-521 | I-522 | I-525 | I-552 |
| I-553 | I-554 | I-555 | I-556 | I-557 |
| I-558 | I-563 | I-564 | I-566 | I-567 |
| I-568 | I-571 | I-572 | I-573 | I-574 |
| I-575 | I-577 | I-578 | I-579 | I-580 |
| I-581 | I-582 | I-583 | I-606 | I-607 |
| I-608 | I-609 | I-610 | I-611 | I-612 |
| I-613 | I-614 | I-615 | I-639 | I-650 |
| I-651 | I-652 | I-657 | I-658 | I-667 |
| I-668 | I-669 | I-671 | I-672 | I-673 |
| I-674 | I-675 | I-676 | I-689 | I-690 |
| I-745 | I-746 | I-748 | I-757 | I-762 |
| I-763 | I-764 | I-765 | I-767 | I-768 |
| I-769 | I-770 | I-772 | I-774 | I-775 |
| I-776 | I-777 | I-787 | I-790 | I-791 |
| I-793 | I-795 | I-796 | I-797 | I-798 |
| I-799 | I-800 | I-801 | I-818 | I-819 |
| I-821 | I-822 | I-824 | I-834 | I-835 |
| I-837 | I-853 | I-854 | I-855 | I-856 |
| I-857 | I-873 | I-874 | I-875 | I-876 |
| I-920 | I-938 | I-976 | I-977 | I-978 |
| I-979 | I-491 | I-1651 | I-1872 | |

### Example 46

### (2r,4r)-N-(4-chloro-1-cyclopentyl-2,3-dihydro-1H-inden-1-yl)-6,8-dioxo-5,7-diazaspiro[3.4]octane-2-carboxamide

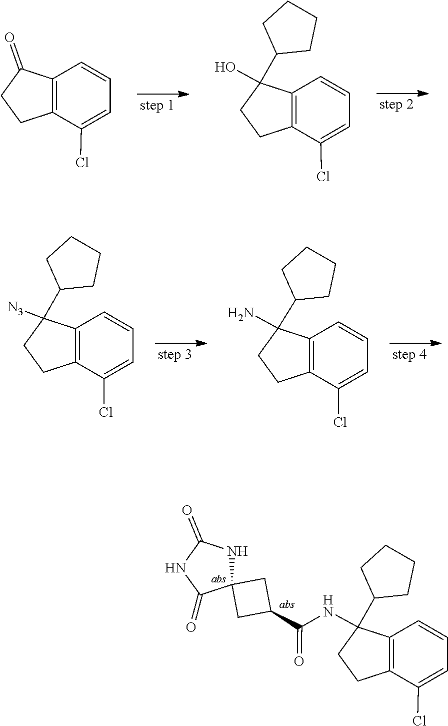 [SMILES: O=C1CCc2c(Cl)cccc21 ; OC1(C2CCCC2)CCc2c(Cl)cccc21 ; [N-]=[N+]=NC1(C2CCCC2)CCc2c(Cl)cccc21 ; NC1(C2CCCC2)CCc2c(Cl)cccc21 ; O=C1NC(=O)[C@]2(C[C@@H](C(=O)NC3(C4CCCC4)CCc4c(Cl)cccc43)C2)N1]

### Step 1. 4-chloro-1-cyclopentyl-2,3-dihydro-1H-inden-1-ol

A flame-dried round-bottomed flask equipped with a magnetic stirrer bar and capped with a rubber septum was charged with a solution of 4-chloro-2,3-dihydro-1H-inden-1-one (83 mg, 0.50 mmol) in THF (1.00 mL). This solution was added dropwise to a separate flame-dried round-bottomed flask containing a stirring solution of LaCl₃·2LiCl (0.83 mL, 0.6 M in THF, 0.50 mmol) at ambient temperature. The resulting mixture was stirred at ambient temperature for 1 hour, then cooled to 0° C. with stirring. A solution of cyclopentylmagnesium bromide (0.28 mL, 2.0 M in Et₂O, 0.55 mmol) was then added dropwise, and the reaction mixture was stirred at 0° C. for ca. 45 minutes. An additional portion of cyclopentylmagnesium bromide (0.14 mL, 2.0 M in Et₂O, 0.28 mmol) was added dropwise to the reaction mixture after this time, and the mixture was stirred at 0° C. for a further ca. 30 minutes. The reaction was then quenched at 0° C. by slow dropwise addition of saturated aqueous NH₄Cl solution (0.5 mL). Water (0.5 mL) was added to dissolve the precipitated inorganic salts, and the mixture was warmed to ambient temperature with vigorous stirring. The mixture was further diluted with water (20 mL) and the organics were extracted with diethyl ether (3×10 mL). The combined organics were washed with saturated aqueous NaCl solution and dried over MgSO₄, filtered and concentrated in vacuo to give the crude product. Purification by flash chromatography on silica gel (eluent: EtOAc in hexanes, 0:1 to 20:80) afforded 4-chloro-1-cyclopentyl-2,3-dihydro-1H-inden-1-ol (68 mg, 0.29 mmol) as a viscous colorless oil. LCMS RT 1.43 min, (M−OH)⁺ 219.1, LCMS method K. ¹H NMR (400 MHz, CDCl₃) δ 7.25-7.22 (m, 2H), 7.20-7.15 (m, 1H), 3.04 (ddd, J=16.8, 9.0, 4.5 Hz, 1H), 2.84 (ddd, J=16.5, 8.3, 6.5 Hz, 1H), 2.42-2.34 (m, 2H), 2.06 (ddd, J=13.5, 9.0, 6.5 Hz, 1H), 1.83-1.76 (m, 2H), 1.70-1.63 (m, 1H), 1.62-1.49 (m, 5H), 1.32-1.22 (m, 1H).

### Step 2. 1-azido-4-chloro-1-cyclopentyl-2,3-dihydro-1H-indene

A flame-dried round-bottomed flask equipped with a magnetic stirrer bar and capped with a rubber septum was charged with a solution of 4-chloro-1-cyclopentyl-2,3-dihydro-1H-inden-1-ol (58 mg, 0.24 mmol) in anhydrous chloroform (0.70 mL), and the solution was cooled to 0° C. with stirring. To the cooled solution was added solid sodium azide (32 mg, 0.49 mmol) in small portions, followed by slow dropwise addition of trifluoroacetic acid (0.12 mL, 1.60 mmol). The reaction mixture was then warmed to 30° C. with stirring for ca. 2 h. The reaction mixture was then cooled to ambient temperature and carefully quenched under nitrogen with a 10% aqueous solution of NH₄OH until the pH was approximately equal to 8-9. The mixture was then poured into a separatory funnel and extracted with chloroform (3×10 mL). The combined organics were then dried over MgSO₄, filtered and concentrated in vacuo to afford crude 1-azido-4-chloro-1-cyclopentyl-2,3-dihydro-1H-indene, which was utilized immediately in the next step assuming quantitative yield.

### Step 3. 4-chloro-1-cyclopentyl-2,3-dihydro-1H-inden-1-amine

The crude azide was dissolved in THF (2.40 mL) with stirring, and a solution of trimethylphosphine (0.26 mL, 1.0 M in THF, 0.26 mmol) was added dropwise at ambient temperature, followed by dropwise addition of water (0.24 mL). The reaction mixture was then heated to 30° C. with stirring for ca. 18 h. The reaction mixture was then cooled to ambient temperature and diluted with EtOAc (10 mL). The phases were separated, and the organic phase was washed with saturated aqueous NaHCO₃ solution (3×5 mL) and saturated aqueous NaCl solution. The organics were then dried over MgSO₄, filtered and concentrated in vacuo to afford crude 4-chloro-1-cyclopentyl-2,3-dihydro-1H-inden-1-amine, which was utilized immediately in the next step assuming quantitative yield. LCMS RT 0.89 min, [M−NH₂]⁺ 219.2, LCMS method K.

### Step 4. (2r,4r)-N-(4-chloro-1-cyclopentyl-2,3-dihydro-1H-inden-1-yl)-6,8-dioxo-5,7-diazaspiro[3.4]octane-2-carboxamide

The crude amine was dissolved in DMF (2.40 mL) in a round-bottomed flask equipped with a magnetic stirbar at ambient temperature, and (2r,4r)-6,8-dioxo-5,7-diazaspiro[3.4]octane-2-carboxylic acid (47 mg, 0.25 mmol) was added in one portion with stirring. To the mixture were then added dropwise DIPEA (0.17 mL, 0.98 mmol) and a solution of T3P (0.14 mL, 50 wt. % in EtOAc, 0.24 mmol) at ambient temperature, and the reaction mixture was stirred for ca. 1 h. An additional portion of T3P (0.07 mL, 50 wt. % in EtOAc, 0.12 mmol) was added after this time, and the mixture was stirred at ambient temperature for a further ca. 30 mins. The reaction mixture was then diluted with DCM (10 mL), quenched with saturated aqueous NaHCO₃ solution (10 mL), and stirred at ambient temperature overnight. The phases were then separated and the aqueous phase was extracted with DCM (3×10 mL). The combined organics were washed with water (10 ml) and saturated aqueous NaCl solution, dried over MgSO₄, filtered and concentrated in vacuo. The residue was then dissolved in a minimum volume of DMF, loaded onto a 12 g C18 cartridge, and purified by reverse-phase chromatography (mobile phase A: 10 mM ammonium formate in water, mobile phase B: acetonitrile; gradient: 40 to 60% B) to afford (2r,4r)-N-(4-chloro-1-cyclopentyl-2,3-dihydro-1H-inden-1-yl)-6,8-dioxo-5,7-diazaspiro[3.4]octane-2-carboxamide (4 mg) as an amorphous bright yellow solid. LCMS RT 1.21 min, [M+H]⁺ 402.3, LCMS method K. ¹H NMR (400 MHz, DMSO-d6) δ 10.55 (br. s, 1H), 8.61 (s, 1H), 7.86 (s, 1H), 7.24-7.19 (m, 1H), 7.19-7.15 (m, 2H), 3.11 (p, J=9.1 Hz, 1H), 2.99 (ddd, J=16.1, 9.6, 4.8 Hz, 1H), 2.79 (ddd, J=16.5, 9.3, 5.7 Hz, 1H), 2.71-2.43 (overlapping m, 3H), 2.36 (ddd, J=11.6, 8.8, 4.6 Hz, 1H), 2.17-2.06 (m, 3H), 1.80-1.71 (m, 1H), 1.57-1.37 (m, 4H), 1.32-1.13 (m, 2H), 1.08-0.98 (m, 1H).

### Example 47

### 7-(4-(3-chlorophenyl)-4-cyclopentyl-2-oxotetrahydropyrimidin-1(2H)-yl)imidazo[1,5-a]pyridine-3-carboxamide

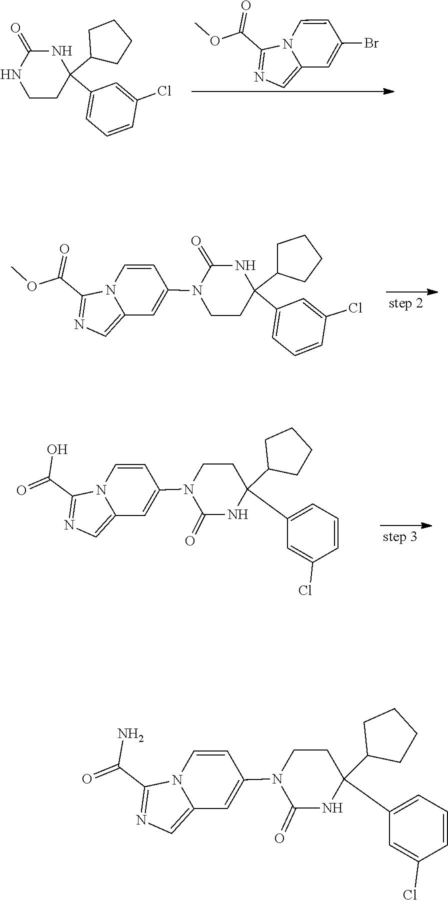 [SMILES: O=C1NCCC(c2cccc(Cl)c2)(C2CCCC2)N1 ; COC(=O)c1ncc2cc(Br)ccn12 ; COC(=O)c1ncc2cc(N3CCC(c4cccc(Cl)c4)(C4CCCC4)NC3=O)ccn12 ; O=C(O)c1ncc2cc(N3CCC(c4cccc(Cl)c4)(C4CCCC4)NC3=O)ccn12 ; NC(=O)c1ncc2cc(N3CCC(c4cccc(Cl)c4)(C4CCCC4)NC3=O)ccn12]

### Step 1. Synthesis of methyl 7-(4-(3-chlorophenyl)-4-cyclopentyl-2-oxotetrahydropyrimidin-1(2H)-yl)imidazo[1,5-a]pyridine-3-carboxylate

A round bottomed flask was charged with 4-(3-chlorophenyl)-4-cyclopentyltetrahydropyrimidin-2(1H)-one (100 mg, 359 μmol), methyl 7-bromoimidazo[1,5-a]pyridine-3-carboxylate (91.5 mg, 359 μmol), Pd-PEPPSI-IPentCl (105 mg, 108 μmol), Cs₂CO₃ (351 mg, 1.08 mmol) and a stirbar. 1,4-Dioxane (1 mL) was added, and the solution was stirred for 4 hours at 90° C. The residue was purified by reverse phase flash chromatography (column: C18 silica gel; mobile phase A: water, mobile phase B: acetonitrile; gradient: 0% to 100% in 10 minutes; detector: UV 220 nm) to give methyl 7-(4-(3-chlorophenyl)-4-cyclopentyl-2-oxotetrahydropyrimidin-1(2H)-yl)imidazo[1,5-a]pyridine-3-carboxylate (30 mg, 66 μmol) as a yellow amorphous solid. LCMS RT 0.988 min, [M+H]⁺ 453.20, LCMS method C.

### Step 2. Synthesis of 7-(4-(3-chlorophenyl)-4-cyclopentyl-2-oxotetrahydropyrimidin-1(2H)-yl)imidazo[1,5-a]pyridine-3-carboxylic acid

A round bottomed flask was charged with methyl 7-(4-(3-chlorophenyl)-4-cyclopentyl-2-oxotetrahydropyrimidin-1(2H)-yl)imidazo[1,5-a]pyridine-3-carboxylate (30 mg, 66 μmol), NaOH (0.33 mL, 2 molar, 0.66 mmol) and a stirbar. MeOH (1 mL) was added, and the solution was stirred for 1 hour at 25° C. The residue was purified by reverse phase flash chromatography:(column: C18 silica gel; mobile phase A: water, mobile phase B: acetonitrile; gradient: 0% to 100% in 10 minutes; detector: UV 220 nm) to give 7-(4-(3-chlorophenyl)-4-cyclopentyl-2-oxotetrahydropyrimidin-1(2H)-yl)imidazo[1,5-a]pyridine-3-carboxylic acid (25 mg, 57 μmol) as a yellow amorphous solid, which was used in the next step without purification.

### Step 3. Synthesis of 7-(4-(3-chlorophenyl)-4-cyclopentyl-2-oxotetrahydropyrimidin-1(2H)-yl)imidazo[1,5-a]pyridine-3-carboxamide

A round bottomed flask was charged with 7-(4-(3-chlorophenyl)-4-cyclopentyl-2-oxotetrahydropyrimidin-1(2H)-yl)imidazo[1,5-a]pyridine-3-carboxylic acid (25 mg, 57 μmol), NH₄Cl (3.0 mg, 57 μmol), HATU (32 mg, 85 μmol), NaHCO₃ (14 mg, 0.17 mmol) and a stirbar. DMF (1 mL) was added, and the solution was stirred for 1 hour at 25° C. The resulting crude material was purified by chiral Pre-HPLC (Column: (R, R) WHELK-01, 4.6*50 mm, 3.5 μm; Mobile Phase A: Hex(0.2% IPAmine): EtOH=80:20; Flow rate: 1 mL/min; Gradient: 0% B to 0% B; Injection Volume: 5 μl mL). Lyophilization yielded 7-(4-(3-chlorophenyl)-4-cyclopentyl-2-oxotetrahydropyrimidin-1(2H)-yl)imidazo[1,5-a]pyridine-3-carboxamide (7.8 mg, 18 μmol, 31%) as an off-white amorphous solid. LCMS RT 0.903 min, [M+H]⁺ 438.15, LCMS method C.

### Example 48

### (S)—N-((1S,3S)-3-acetamidocyclopentyl)-2-(3-chloro-2,6-difluorophenyl)-2-(4-fluorobicyclo[2.2.1]heptan-1-yl)acetamide

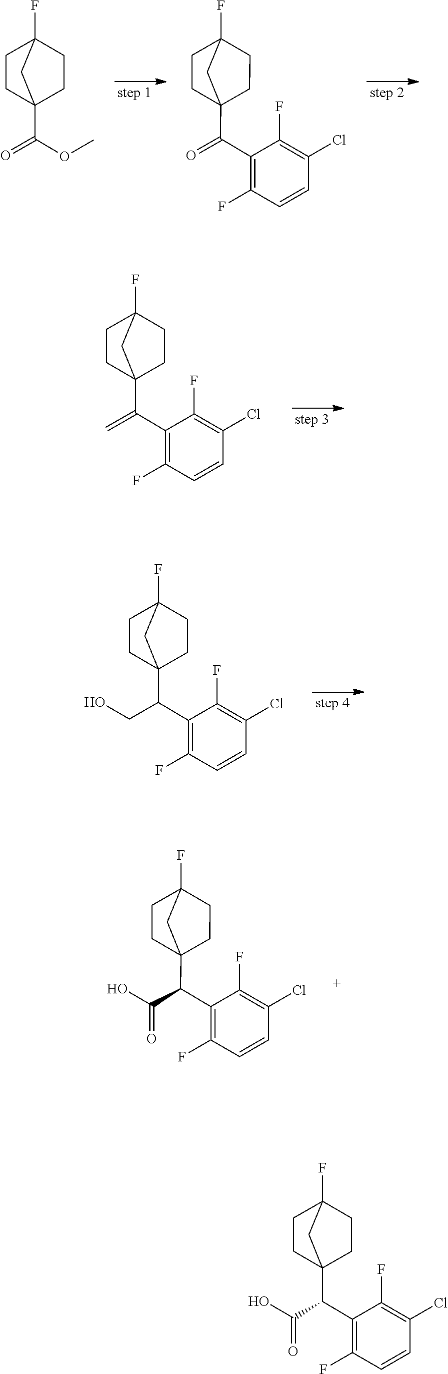 [SMILES: COC(=O)C12CCC(F)(CC1)C2 ; O=C(c1c(F)ccc(Cl)c1F)C12CCC(F)(CC1)C2 ; C=C(c1c(F)ccc(Cl)c1F)C12CCC(F)(CC1)C2 ; OCC(c1c(F)ccc(Cl)c1F)C12CCC(F)(CC1)C2 ; O=C(O)[C@H](c1c(F)ccc(Cl)c1F)C12CCC(F)(CC1)C2 ; O=C(O)[C@@H](c1c(F)ccc(Cl)c1F)C12CCC(F)(CC1)C2]

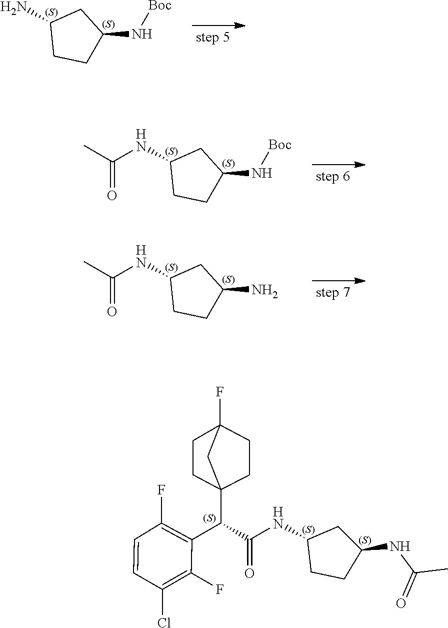

### Step 1. Synthesis of (3-chloro-2,6-difluorophenyl)(4-fluorobicyclo[2.2.1]heptan-1-yl)methanone

n-BuLi (2.5 M, 61.0 mL) was diluted with THF (175 mL). A solution of 1-chloro-2,4-difluorobenzene (18.1 g, 122 mmol) in THF (100 mL) was added dropwise at −78° C. under N₂. After stirring at −78° C. for 2 hours, a solution of methyl 4-fluorobicyclo[2.2.1]heptane-1-carboxylate (17.5 g, 102 mmol) in THF (175 mL) was added dropwise at −78° C. under N₂. The mixture was stirred at −78° C. for 4 hours. The reaction mixture was poured into sat. NH₄Cl solution (350 mL) and extracted with ethyl acetate (200 mL*2). The combined organic layers were washed with brine (200 mL), dried over Na₂SO₄, filtered and concentrated to give a residue. The residue was purified by column chromatography (silica gel, petroleum ether:ethyl acetate 1:0 to 0:1) to give (3-chloro-2,6-difluorophenyl)(4-fluorobicyclo[2.2.1]heptan-1-yl)methanone as a yellow oil. ¹H NMR: (400 MHz, CDCl₃) δ 7.43 (dt, J=5.6, 8.6 Hz, 1H), 6.93 (ddd, J=1.6, 7.8, 9.0 Hz, 1H), 2.29-2.15 (m, 2H), 2.06-1.93 (m, 4H), 1.92-1.77 (m, 4H).

### Step 2. Synthesis of 1-(1-(3-chloro-2,6-difluorophenyl)vinyl)-4-fluorobicyclo[2.2.1]heptane

To a solution of Ph₃PMeBr (16.3 g, 45.7 mmol) in THF (66.0 mL) was added t-BuOK (1.0 M, 45.7 mL) at 0° C. The mixture was warmed to 15° C. and stirred at 15° C. for 2 hours. Then a solution of (3-chloro-2,6-difluorophenyl)(4-fluorobicyclo[2.2.1]heptan-1-yl)methanone (6.60 g, 22.9 mmol) in THF (66.0 mL) was added at 0° C. The mixture was stirred at 0° C. for 2 hours, then warmed to 15° C. and stirred at 15° C. for 12 hours. The reaction was quenched by addition of water (6.00 mL) and filtered. The filtrate was concentrated under reduced pressure to give a residue. The residue was purified by column chromatography (silica gel, petroleum ether:ethyl acetate 1:0 to 10:1) to give 1-(1-(3-chloro-2,6-difluorophenyl)vinyl)-4-fluorobicyclo[2.2.1]heptane (5.60 g, 17.3 mmol) as a yellow oil. ¹H NMR: (400 MHz, CDCl₃)

δ 7.31 (dt, J=5.6, 8.4 Hz, 1H), 6.87 (br d, J=0.6 Hz, 1H), 5.43 (s, 1H), 5.06 (s, 1H), 2.02-1.89 (m, 4H), 1.83-1.74 (m, 4H), 1.70-1.61 (m, 2H).

### Step 3. Synthesis of 2-(3-chloro-2,6-difluorophenyl)-2-(4-fluorobicyclo[2.2.1]heptan-1-yl)ethan-1-ol

To a solution of 1-(1-(3-chloro-2,6-difluorophenyl)vinyl)-4-fluorobicyclo[2.2.1]heptane (5.50 g, 19.2 mmol) in THF (165 mL) was added BH₃-Me₂S (3.84 mL) at 25° C. under N₂. The mixture was heated to 50° C. and stirred at 50° C. 1 hour. MeOH (18.7 mL) was added dropwise at 0° C. After that NaOH (2 M, 28.8 mL) was added dropwise at 0° C., then H₂O₂ (30%, 9.28 mL, 96.6 mmol) was added at 0° C. slowly. The mixture was stirred at 0° C. for 1.5 hours. The mixture was poured into sat. Na₂S₂O₃ aqueous solution (200 mL) slowly, stirred for 10 minutes, and extracted with ethyl acetate (200 mL*2). The combined organic phases were washed with water (200 mL), brine (200 mL), dried over Na₂SO₄, filtered and concentrated. The residue was purified by column chromatography (silica gel, petroleum ether:ethyl acetate 1:0 to 0:1) to give 2-(3-chloro-2,6-difluorophenyl)-2-(4-fluorobicyclo[2.2.1]heptan-1-yl)ethan-1-ol (4.80 g, 15.8 mmol) as a yellow oil. ¹H NMR: (400 MHz, DMSO) δ 7.52 (dt, J=5.6, 8.6 Hz, 1H), 7.19-7.05 (m, 1H), 4.72-4.63 (m, 1H), 3.93-3.77 (m, 2H), 3.43-3.35 (m, 1H), 1.88-1.57 (m, 7H), 1.52-1.29 (m, 3H).

### Step 4. Synthesis of (R)-2-(3-chloro-2,6-difluorophenyl)-2-(4-fluorobicyclo[2.2.1]heptan-1-yl)acetic acid and (S)-2-(3-chloro-2,6-difluorophenyl)-2-(4-fluorobicyclo[2.2.1]heptan-1-yl)acetic acid

To a solution of 2-(3-chloro-2,6-difluorophenyl)-2-(4-fluorobicyclo[2.2.1]heptan-1-yl)ethan-1-ol (4.80 g, 15.8 mmol) in acetonitrile (76.0 mL) was added a solution of NaClO₂ (11.4 g, 126 mmol) in H₂O (14.0 mL) at 0° C. Then TEMPO (297 mg, 1.89 mmol), a solution of Na₂HPO₄ (0.67 M, 23.5 mL) and NaH₂PO₄ (0.67 M, 23.5 mL) in water, and a solution of NaClO (2.35 g, 1.89 mmol, 1.94 mL) in H₂O (14.0 mL) was added at 0° C. The mixture was warmed to 15° C. and stirred at 15° C. for 12 hours. The reaction mixture was cooled to 0° C. Water (200 mL) was added, followed by Na₂SO₃ (28.4 g) at 0° C. The mixture was stirred at 15° C. for 30 minutes. The pH was adjusted to 1-2 with H₃PO₄, and the solution was extracted with ethyl acetate (200 mL*2). The combined organic layers were washed with brine (200 mL), dried over Na₂SO₄, filtered and concentrated under reduced pressure. The residue was purified by column chromatography (silica gel, petroleum ether:ethyl acetate 1:0 to 0:1) to give 2-(3-chloro-2,6-difluorophenyl)-2-(4-fluorobicyclo[2.2.1]heptan-1-yl)acetic acid as a white solid. It was further purified by chiral SFC (column: DAICEL CHIRALPAK AS 250 mm*30 mm, 10 μm); mobile phase: [CO₂-ⁱPrOH]; gradient: 15% ⁱPrOH isocratic) to give (R)-2-(3-chloro-2,6-difluorophenyl)-2-(4-fluorobicyclo[2.2.1]heptan-1-yl)acetic acid and (S)-2-(3-chloro-2,6-difluorophenyl)-2-(4-fluorobicyclo[2.2.1]heptan-1-yl)acetic acid.

Isomer 1: 2.00 g, 6.28 mmol was obtained as a white solid. ¹H NMR: (400 MHz, CDCl₃) δ 7.37 (dt, J=5.6, 8.6 Hz, 1H), 6.93 (dt, J=1.6, 9.0 Hz, 1H), 4.21 (s, 1H), 2.07-1.91 (m, 3H), 1.91-1.80 (m, 3H), 1.80-1.68 (m, 2H), 1.67-1.56 (m, 2H).

Isomer 2: 2.01 g, 6.28 mmol was obtained as a white solid. ¹H NMR: (400 MHz, CDCl₃) δ 7.37 (dt, J=5.6, 8.6 Hz, 1H), 6.93 (dt, J=1.6, 9.0 Hz, 1H), 4.20 (s, 1H), 2.07-1.93 (m, 3H), 1.92-1.81 (m, 3H), 1.81-1.70 (m, 2H), 1.68-1.57 (m, 2H).

### Step 5. Synthesis of tert-butyl ((1S,3S)-3-acetamidocyclopentyl) carbamate

To a mixture of tert-butyl ((1S,3S)-3-aminocyclopentyl) carbamate (500 mg, 2.50 m mol) and TEA (1.04 mL, 7.49 mmol) in DCM (8 mL) was added Ac₂O (283 μL, 3.00 mmol) dropwise at 0° C. under a nitrogen atmosphere. The mixture was stirred for 2 hours at room temperature. The mixture was concentrated. The resulting crude material was purified by re verse phase flash chromatography (column: C18 silica gel; mobile phase A: water, mobile p hase B: acetonitrile; gradient: 0% to 100% B in 20 min; detector: UV 200 nm) to give tert-butyl ((1S,3S)-3-acetamidocyclopentyl) carbamate (300 mg, 1.24 mmol) as an off-white solid. LCMS RT 0.738 min, [M+H]⁺ 243.15, LCMS method B.

### Step 6. Synthesis of N-((1S,3S)-3-aminocyclopentyl) acetamide

A mixture of tert-butyl ((1S,3S)-3-acetamidocyclopentyl) carbamate (120 mg, 495 μmol) in DCM:TFA (2:1, 1 mL) was stirred for 2 hours at room temperature. The mixture was concentrated in vacuo to give N-((1S,3S)-3-aminocyclopentyl)acetamide (60 mg, 0.42 mmol as a colorless oil. LCMS RT 0.158 min, [M+H]⁺ 142.00, LCMS method B.

### Step 7. Synthesis of (S)—N-((1S,3S)-3-acetamidocyclopentyl)-2-(3-chloro-2,6-difluorophenyl)-2-(4-fluorobicyclo[2.2.1]heptan-1-yl) acetamide

To a mixture of (S)-2-(3-chloro-2,6-difluorophenyl)-2-(4-fluorobicyclo[2.2.1]heptan-1-yl) acetic acid (25 mg, 78 μmol), N-((1S,3S)-3-aminocyclopentyl) acetamide (13 mg, 94 μmol) and NaHCO₃ (33 mg, 0.39 mmol) in DMF (1 mL) was added HATU (60 mg, 0.16 mmol). The mixture was stirred for 6 hours at 25° C. The reaction mixture was diluted with water (10 mL), and the aqueous phase was extracted with ethyl acetate (20 mL) three times. The combined organic layers were washed with brine, dried over sodium sulfate, filtered and concentrated in vacuo. The resulting crude material was purified by preparative HPLC (column: XBridge Prep OBD C18 Column, 30*150 mm, 5 μm; mobile phase A: water (10 mM NH₄HCO₃)+0.05% NH₄OH, mobile phase B: acetonitrile; flow rate: 60 mL/min; gradient: 31% B to 58% B in 7 min; wavelength: 254/220 nm; RT (min): 7.62) to give (S)—N-((1S,3S)-3-acetamidocyclopentyl)-2-(3-chloro-2,6-difluorophenyl)-2-(4-fluorobicyclo[2.2.1]heptan-1-yl)acetamide (3.6 mg, 8.1 μmol) as an off-white amorphous solid. ¹HNMR (400 MHz, DMSO-d6) δ 7.83 (s, 1H), 7.69 (d, J=7.3 Hz, 1H), 7.58 (td, J=8.7, 5.5 Hz, 1H), 7.15 (td, J=9.3, 1.6 Hz, 1H), 4.13 (p, J=7.1 Hz, 1H), 3.97 (p, J=7.0 Hz, 1H), 3.86 (s, 1H), 1.94-1.76 (m, 5H), 1.70 (d, J=28.3 Hz, 9H), 1.62-1.38 (m, 3H), 1.38-1.14 (m, 2H). LCMS RT 1.008 min, [M+H]⁺ 443.25, LCMS method D.

Additional compounds prepared according to the methods of Example 48 are listed in Table 11 below. Corresponding ¹H NMR and mass spectrometry characterization for these compounds are described in Table 1. Certain compounds in Table 11 below were prepared with other compounds whose preparation is described further below in the Examples.

TABLE 11
Additional exemplary compounds

| | | | | |
|---|---|---|---|---|
| I-272 | I-273 | I-297 | I-298 | I-3720 |
| I-3722 | | | | |

### Example 49

### (2r,4S)—N—((S)-2-amino-1-(3-chlorophenyl)-1-cyclopentyl-2-oxoethyl)-6,8-dioxo-5,7-diazaspiro[3.4]octane-2-carboxamide

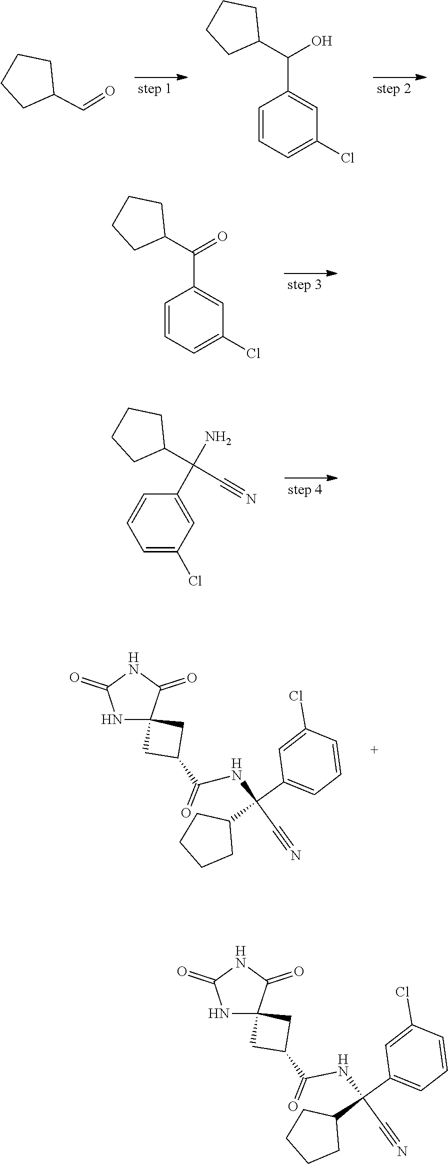 [SMILES: O=CC1CCCC1 ; OC(c1cccc(Cl)c1)C1CCCC1 ; O=C(c1cccc(Cl)c1)C1CCCC1 ; N#CC(N)(c1cccc(Cl)c1)C1CCCC1 ; N#C[C@@](NC(=O)[C@H]1C[C@@]2(C1)NC(=O)NC2=O)(c1cccc(Cl)c1)C1CCCC1 ; N#C[C@](NC(=O)[C@H]1C[C@@]2(C1)NC(=O)NC2=O)(c1cccc(Cl)c1)C1CCCC1]

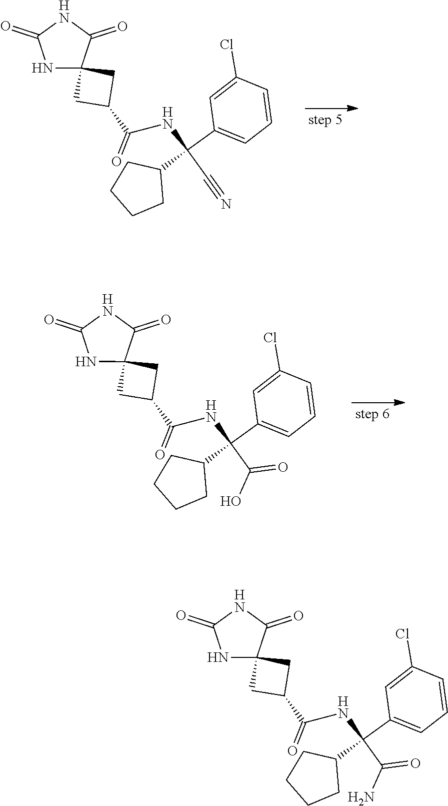 [SMILES: N#C[C@@](NC(=O)[C@H]1C[C@@]2(C1)NC(=O)NC2=O)(c1cccc(Cl)c1)C1CCCC1 ; O=C1NC(=O)[C@]2(C[C@@H](C(=O)N[C@](C(=O)O)(c3cccc(Cl)c3)C3CCCC3)C2)N1 ; NC(=O)[C@@](NC(=O)[C@H]1C[C@@]2(C1)NC(=O)NC2=O)(c1cccc(Cl)c1)C1CCCC1]

### Step 1. Synthesis of (3-chlorophenyl)(cyclopentyl)methanol

To a mixture of cyclopentanecarbaldehyde (3.92 g, 0.040 mol) in THF (30 mL) was added (3-chlorophenyl)magnesium bromide (1 M in THF, 40 ml, 0.040 mol) dropwise at −78° C. under a nitrogen atmosphere. The mixture was stirred for 2 hours at −78° C. The reaction was quenched with saturated NH₄Cl (aq.) and the aqueous phase was extracted with ethyl acetate (100 mL) three times. The combined organic layers were washed with brine, dried over sodium sulfate, filtered and concentrated in vacuo. The residue was purified by C₁₈ flash chromatography (CH₃CN/water) to afford (3-chlorophenyl)(cyclopentyl)methanol (2.74 g, 0.013 mol, 30%) as a yellow oil. LCMS RT 1.003 min, [M+H]⁺ not observed, LCMS method C.

### Step 2. Synthesis of (3-chlorophenyl)(cyclopentyl)methanone

To a mixture of (3-chlorophenyl)(cyclopentyl)methanol (1.05 g, 5.0 mmol) and molecular sieve 4 Å (5.0 g) in DCM (10 mL) was added PCC (1.29 g, 6.0 mmol) in portions at 0° C. under a nitrogen atmosphere. The mixture was stirred for 2 hours at 25° C. The reaction mixture was filtered through Celite, the pad was washed with DCM, and the filtrate was concentrated in vacuo to give (3-chlorophenyl)(cyclopentyl)methanone (1.22 g, 5.85 mmol) as a yellow oil. ¹H NMR 17 (400 MHz, DMSO-d₆) δ 7.93 (dt, J=6.0, 1.6 Hz, 2H), 7.76-7.65 (m, 1H), 7.56 (t, J=8.1 Hz, 1H), 3.83 (tt, J=8.8, 6.8 Hz, 1H), 1.88 (ddt, J=12.7, 8.8, 6.4 Hz, 2H), 1.78-1.66 (m, 2H), 1.70-1.54 (m, 4H).

### Step 3. Synthesis of 2-amino-2-(3-chlorophenyl)-2-cyclopentylacetonitrile

A mixture of (3-chlorophenyl)(cyclopentyl)methanone (1.04 g, 5.0 mmol), TMSCN (1.98 g, 0.02 mol) and NH₃ (10 mL, 7 N in MeOH) was stirred for 16 hours at 90° C. The reaction mixture was concentrated in vacuo. The residue was purified by C₁₈ flash chromatography (CH₃CN/H₂O) to afford 2-amino-2-(3-chlorophenyl)-2-cyclopentylacetonitrile (600 mg, 2.56 mmol) as a yellow oil. LCMS RT 0.870 min, [M+H]⁺ 235, LCMS method C.

### Step 4. Synthesis of (2r,4S)—N—((S)-(3-chlorophenyl)(cyano)(cyclopentyl)methyl)-6,8-dioxo-5,7-diazaspiro[3.4]octane-2-carboxamide and (2r,4R)—N—((R)-(3-chlorophenyl)(cyano)(cyclopentyl)methyl)-6,8-dioxo-5,7-diazaspiro[3.4]octane-2-carboxamide

A mixture of 2-amino-2-(3-chlorophenyl)-2-cyclopentylacetonitrile (500 mg, 2.13 m mol), (2r,4r)-6,8-dioxo-5,7-diazaspiro[3.4]octane-2-carboxylic acid (392 mg, 2.13 mmol), T EA (891 μL, 6.39 mmol) and T₃P (1.02 g, 3.20 mmol) in DMF (5 mL) was stirred for 1 hour at room temperature. The reaction mixture was diluted with water (30 mL), and the aqueous phase was extracted with ethyl acetate (50 mL) three times. The combined organic layers w ere washed with brine, dried over sodium sulfate, filtered and concentrated in vacuo. The resulting crude material was purified by C18 flash chromatography to afford (2r,4r)-N-((3-chlo-rophenyl)(cyano)(cyclopentyl)methyl)-6,8-dioxo-5,7-diazaspiro[3.4]octane-2-carboxamide as a white solid. LCMS RT 0.855 min, [M+H]⁺ 401, LCMS method D.

The product was further purified by chiral preparative HPLC (column: DZ-CHIRALPAK IH-3, 4.6*50 mm, 3.0 μm; mobile phase A: hexane; mobile phase B: EtOH; flow rate: 1 mL/min; gradient: 20% B isocratic; injection volume: 5 mL) to give (2r,4S)—N—((S)-(3-chlorophenyl)(cyano)(cyclopentyl)methyl)-6,8-dioxo-5,7-diazaspiro[3.4]octane-2-carboxamide and (2r,4R)—N—((R)-(3-chlorophenyl)(cyano)(cyclopentyl)methyl)-6,8-dioxo-5,7-diazaspiro[3.4]octane-2-carboxamide, both as an off-white amorphous solid.

Isomer 1: 10 mg. ¹H NMR (400 MHz, DMSO-d₆) δ 10.59 (s, 1H), 8.99 (s, 1H), 8.64 (s, 1H), 7.50-7.38 (m, 2H), 7.35 (dt, J=4.6, 1.9 Hz, 2H), 3.34 (s, 1H), 2.68 (t, J=10.9 Hz, 1H), 2.44 (t, J=8.6 Hz, 1H), 2.22 (dd, J=12.8, 9.0 Hz, 2H), 2.05 (dd, J=13.4, 8.0 Hz, 1H), 1.62-1.48 (m, 4H), 1.43-1.13 (m, 4H). LCMS RT 0.838 min, [M+H]⁺ 401.10, LCMS method C.

Isomer 2: 5 mg. LCMS 1.318 min, [M+H]⁺ 401.10, LCMS method B. ¹H NMR (400 MHz, DMSO-d₆) δ 10.60 (s, 1H), 8098 (s, 1H), 8.64 (s, 1H), 7.34-7.47 (m, 4H), 3.24 (t, J=8.8 Hz, 1H), 2.66-2.69 (m, 1H), 2.40-2.58 (m, 2H), 2.19-2.30 (m, 2H), 2.01-2.08 (m, 1H), 1.40-1.72 (m, 5H), 1.10-1.29 (m, 2H).

### Step 5. Synthesis of (S)-2-(3-chlorophenyl)-2-cyclopentyl-2-((2r,4S)-6,8-dioxo-5,7-diazaspiro[3.4]octane-2-carboxamido)acetic acid

A mixture of (2r,4S)—N—((S)-(3-chlorophenyl)(cyano)(cyclopentyl)methyl)-6,8-dioxo-5,7-diazaspiro[3.4]octane-2-carboxamide (85 mg, 0.21 mmol) and HCl (5 mL, 12 N) was stirred for 1 h at 40° C. After cooling to room temperature, the reaction mixture was extracted with dichloromethane (20 mL) three times. The combined organic layers were washed with brine, dried over sodium sulfate, filtered and concentrated in vacuo to afford (S)-2-(3-chlorophenyl)-2-cyclopentyl-2-((2r,4S)-6,8-dioxo-5,7-diazaspiro[3.4]octane-2-carboxamido)acetic acid (70 mg, 0.17 mmol) as a colorless oil. LCMS RT 0.820 min, [M+H]⁺ 420.0, LCMS method D.

### Step 6. Synthesis of (2r,4S)—N—((S)-2-amino-1-(3-chlorophenyl)-1-cyclopentyl-2-oxoethyl)-6,8-dioxo-5,7-diazaspiro[3.4]octane-2-carboxamide

A mixture of (S)-2-(3-chlorophenyl)-2-cyclopentyl-2-((2r,4S)-6,8-dioxo-5,7-diazaspiro[3.4]octane-2-carboxamido)acetic acid (75 mg, 0.18 mmol), DIEA (93 μL, 0.54 mmol), HATU (0.10 g, 0.27 mmol) and NH₄Cl (10 mg, 0.20 mmol) in DMF (2 mL) was stirred for 1 hour at room temperature. The reaction mixture was diluted with water (10 mL), and the aqueous phase was extracted with ethyl acetate (10 mL) three times. The combined organic layers were washed with brine, dried over sodium sulfate, filtered and concentrated in vacuo. The resulting crude material was purified by C18 flash chromatography (CH₃CN/H₂O) to give (2r,4S)—N—((S)-2-amino-1-(3-chlorophenyl)-1-cyclopentyl-2-oxoethyl)-6,8-dioxo-5,7-diazaspiro[3.4]octane-2-carboxamide (50 mg, 0.12 mmol) as colorless oil. ¹H NMR (400 MHz, DMSO-d₆) 10.59 (s, 1H), 8.58 (s, 1H), 7.95 (s, 1H), 7.54 (s, 1H), 7.37 (s, 1H), 7.28 (d, J=13.4 Hz, 2H), 7.18 (s, 1H), 7.10 (s, 1H), 2.72 (s, 2H), 2.62 (s, 1H), 2.28 (d, J=12.5 Hz, 2H), 1.58 (s, 1H), 1.42 (s, 8H). LCMS RT 0.715 min, [M+H]⁺ 419, LCMS method C.

### Example 50

### (S)-2-(2-(((3-chloro-2,6-difluorophenyl)(cyclopentyl)methyl)amino)phenyl)ethan-1-ol

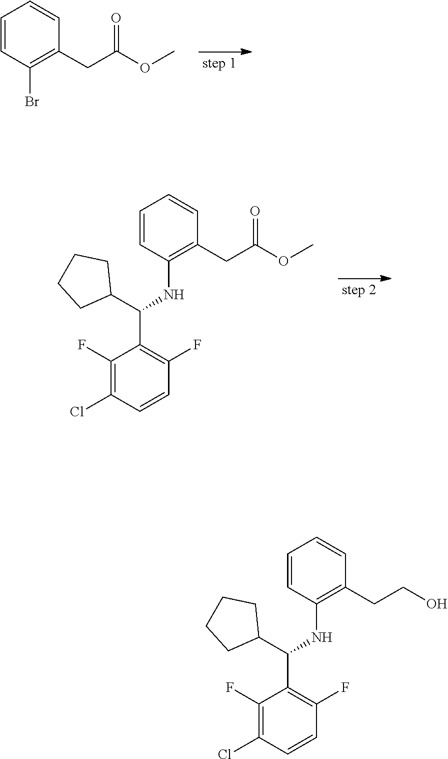 [SMILES: COC(=O)Cc1ccccc1Br ; COC(=O)Cc1ccccc1N[C@H](c1c(F)ccc(Cl)c1F)C1CCCC1 ; OCCc1ccccc1N[C@H](c1c(F)ccc(Cl)c1F)C1CCCC1]

### Step 1. Synthesis of methyl (S)-2-(2-(((3-chloro-2,6-difluorophenyl)(cyclopentyl)methyl)amino)phenyl)acetate

To a mixture of methyl 2-(2-bromophenyl)acetate (400 mg, 1.75 mmol), (S)-(3-chloro-2,6-difluorophenyl)(cyclopentyl)methanamine (429 mg, 1.75 mmol) and Cs₂cO₃ (1.70 g, 5.24 mmol) in toluene (1 mL) was added Pd-PEPPSI-IHept-Cl (CAS: 1814936-54-3) (170 mg, 175 μmol) under a N₂ atmosphere. The mixture was stirred for 16 h at 100° C. After cooling to room temperature, the reaction mixture was diluted with water (50 mL), and the aqueous phase was extracted with ethyl acetate (50 mL*3). The combined organic layers were washed with brine, dried over sodium sulfate, filtered and concentrated in vacuo. The residue was purified by reverse phase flash chromatography (column, C18 silica gel; mobile phase A: water, mobile phase B: acetonitrile; gradient: 0% to 100% in 20 min; detector: UV 220 nm) to give methyl (S)-2-(2-(((3-chloro-2,6-difluorophenyl)(cyclopentyl)methyl)amino)phenyl)acetate (380 mg, 965 μmol) as a yellow oil. LCMS RT 1.530 min, [M+H]⁺ 394.05, LCMS method B.

### Step 2. Synthesis of (S)-2-(2-(((3-chloro-2,6-difluorophenyl)(cyclopentyl)methyl)amino)phenyl)ethan-1-ol

To a mixture of methyl (S)-2-(2-(((3-chloro-2,6-difluorophenyl) (cyclopentyl)methyl) amino)phenyl) acetate (100 mg, 254 μmol) in THF (1 mL) was added LiAlH₄ (19 mg, 508 μmol) in portions at 0° C. The mixture was stirred for 1 h at 25° C. The reaction was quenched with H₂O (19 μl), NaOH (4 N, 38 μl), H₂O (19 μl). The mixture was filtered through a pad of Celite, the pad was washed with ethyl acetate, and the combined filtrate was concentrated in vacuo. The resulting crude material was purified by preparative HPLC (column: XBridge Prep Phenyl OBD Column, 19*250 mm, 5 μm; mobile phase A: water (10 mM NH₄HCO₃), mobile phase B: acetonitrile; flow rate: 25 mL/min; gradient: 55% B to 75% B in 10 min; wavelength: 220 nm; RT1 (min): 9.77) to give (S)-2-(2-(((3-chloro-2,6-difluorophenyl)(cyclopentyl)methyl)amino)phenyl)ethan-1-ol (5 mg, 0.01 mmol) as a colorless oil. ¹H NMR (400 MHz, DMSO-d6) δ 7.52 (td, J=8.8, 5.6 Hz, 1H), 7.14 (td, J=9.5, 1.6 Hz, 1H), 6.97-6.91 (m, 2H), 6.50 (td, J=7.4, 1.1 Hz, 1H), 6.41 (d, J=7.9 Hz, 1H), 5.34 (d, J=8.7 Hz, 1H), 4.85 (t, J=5.0 Hz, 1H), 4.49 (t, J=9.5 Hz, 1H), 3.59 (dt, J=7.1, 5.6 Hz, 2H), 2.70-2.55 (m, 3H), 2.13-2.03 (m, 1H), 1.71-1.34 (m, 6H), 1.11 (dq, J=16.2, 8.2, 6.8 Hz, 1H). LCMS RT 1.383 min, [M+H]⁺ 366.10, LCMS method B.

Additional compounds prepared according to the methods of Example 50 are listed in Table 12 below. Corresponding ¹H NMR and mass spectrometry characterization for these compounds are described in Table 1. Certain compounds in Table 12 below were prepared with other compounds whose preparation is described further below in the Examples.

TABLE 12
other exemplary compounds

| | | | | |
|---|---|---|---|---|
| I-1790 | I-1817 | I-1839 | I-1844 | I-1853 |
| I-1969 | | | | |

### Example 51

### (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea

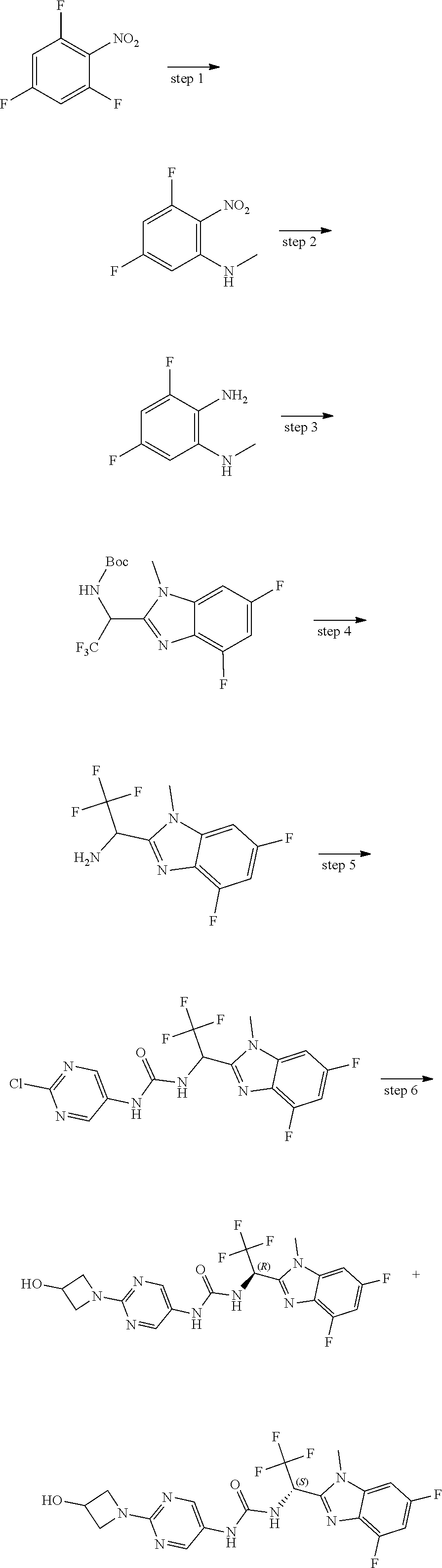

### Step 1. Synthesis of 3,5-difluoro-N-methyl-2-nitroaniline

To a stirred mixture of 1,3,5-trifluoro-2-nitrobenzene (50 mg, 0.28 mmol) and ° C. under a nitrogen atmosphere. The resulting mixture was stirred at 25° C. for 16 hours under nitrogen. The mixture was filtered, and the filter cake was washed with EtOAc (3×50 mL). The filtrate was concentrated under reduced pressure. The residue was purified by silica gel column chromatography, eluting with petroleum ether/EtOAc (10:1) to give 3,5-difluoro-N-methyl-2-nitroaniline (30 mg, 0.16 mmol) as a yellow oil. —H NMR (300 MHz, DMSO-d6) δ 7.73 (s, 1H), 6.70-6.49 (m, 2H), 2.86 (d, J=4.9 Hz, 3H).

### Step 2. Synthesis of 3,5-difluoro-N1-methylbenzene-1,2-diamine

To a stirred mixture of 3,5-difluoro-N-methyl-2-nitroaniline (200 mg, 1.06 mmol) and Zn powder (695 mg, 10.6 mmol) in MeOH (1 mL) was added saturated NH₄Cl solution (1 mL) at room temperature under a nitrogen atmosphere. The resulting mixture was stirred for 3 hours at 50° C. under nitrogen. After cooling to room temperature, the mixture was filtered, and the filter cake was washed with EtOH (3×50 mL). The filtrate was concentrated under reduced pressure. The residue was diluted with water (100 mL) and extracted with DCM (2×100 mL). The combined organic layers were washed with water (1×10 mL) and brine (1×100 mL), and dried over anhydrous Na₂SO₄. After filtration, the filtrate was concentrated under reduced pressure to afford 3,5-difluoro-N1-methylbenzene-1,2-diamine (120 mg, 759 μmol) as a black solid. LCMS RT 1.087 min [M−H]⁻ 157.00, LCMS method E. ¹H NMR (400 MHz, DMSO-d6) δ 6.27 (ddd, J=10.9, 9.1, 2.8 Hz, 1H), 6.08 (ddd, J=11.6, 2.8, 1.6 Hz, 1H), 5.32 (s, 1H), 4.19 (s, 2H), 2.72 (d, J=4.9 Hz, 3H).

### Step 3. Synthesis of tert-butyl (1-(4,6-difluoro-1-methyl-1H-benzo[d]imidazol-2-yl)-2,2,2-trifluoroethyl)carbamate

To a stirred mixture of 3,5-difluoro-N1-methylbenzene-1,2-diamine (200 mg, 1.26 mmol) and 2-((tert-butoxycarbonyl)amino)-3,3,3-trifluoropropanoic acid (308 mg, 1.26 mmol) in DMF (1 mL) were added HATU (736 mg, 1.39 mmol) and TEA (647 mg, 2.53 mmol) at room temperature under a nitrogen atmosphere. The resulting mixture was stirred at 60° C. overnight under nitrogen. After cooling to room temperature, water was added and the mixture was extracted with EtOAc (3×100 mL). The combined organic layers were washed with brine (1×100 mL) and dried over anhydrous Na₂SO₄. After filtration, the filtrate was concentrated under reduced pressure. The residue was purified by silica gel column chromatography, eluting with petroleum ether/EtOAc (10:1) to afford tert-butyl (3-((2,4-difluoro-6-(methylamino)phenyl)amino)-1,1,1-trifluoro-3-oxopropan-2-yl)carbamate (150 mg) as a yellow solid.

A solution of tert-butyl (3-((2,4-difluoro-6-(methylamino)phenyl)amino)-1,1,1-trifluoro-3-oxopropan-2-yl)carbamate (140 mg) in HOAc (2 mL) was stirred for 80° C. at 3 hours under a nitrogen atmosphere. The resulting mixture was concentrated under reduced pressure to afford tert-butyl (1-(4,6-difluoro-1-methyl-1H-benzo[d]imidazol-2-yl)-2,2,2-trifluoroethyl)carbamate (150 mg, 0.25 mmol) as a yellow solid, which was used in the next step without purification.

### Step 4. Synthesis of 1-(4,6-difluoro-1-methyl-1H-benzo[d]imidazol-2-yl)-2,2,2-trifluoroethan-1-amine

To a stirred mixture of tert-butyl (1-(4,6-difluoro-1-methyl-1H-benzo[d]imidazol-2-yl)-2,2,2-trifluoroethyl)carbamate (126 mg, 345 μmol) in DCM (1 mL) was added HCl in 1,4-dioxane (2 mL, 1 M, 2 mmol) at 25° C. under a nitrogen atmosphere. The resulting mixture was stirred for 2 hours at 25° C. under nitrogen. The mixture was concentrated under reduced pressure to afford 1-(4,6-difluoro-1-methyl-1H-benzo[d]imidazol-2-yl)-2,2,2-trifluoroethan-1-amine (140 mg, 528 μmol) as a yellow solid. LCMS RT 1.027 min, [M+H]⁺ 265.95, LCMS method E.

### Step 5. 1-(2-chloropyrimidin-5-yl)-3-(1-(4,6-difluoro-1-methyl-1H-benzo[d]imidazol-2-yl)-2,2,2-trifluoroethyl)urea

To a stirred mixture of 1-(4,6-difluoro-1-methyl-1H-benzo[d]imidazol-2-yl)-2,2,2-trifluoroethan-1-amine (155 mg, 584 μmol) in pyridine (2 mL) was added phenyl (2-chloropyrimidin-5-yl)carbamate (146 mg, 584 μmol) at 25° C. under a nitrogen atmosphere. The resulting mixture was stirred for 16 hours at 80° C. under nitrogen. The resulting mixture was extracted with DCM (2×100 mL). The combined organic layers were washed with brine (3×50 mL) and dried over anhydrous Na₂SO₄. After filtration, the filtrate was concentrated under reduced pressure. The residue was purified by silica gel column chromatography, eluted with petroleum ether/EtOAc (10:1) to afford 1-(2-chloropyrimidin-5-yl)-3-(1-(4,6-difluoro-1-methyl-1H-benzo[d]imidazol-2-yl)-2,2,2-trifluoroethyl)urea (80 mg, 0.19 mmol) as a yellow solid. LCMS RT 1.142 min, [M+H]⁺ 421.05, LCMS method E. H NMR (300 MHz, DMSO-d6) δ 9.35 (s, 1H), 8.82 (d, J=6.4 Hz, 2H), 8.10 (d, J=9.0 Hz, 1H), 7.65-7.45 (m, 1H), 7.27-7.11 (m, 1H), 6.28 (p, J=7.1 Hz, 1H), 3.91 (s, 3H).

### Step 6. Synthesis of (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea

To a stirred mixture of 1-(2-chloropyrimidin-5-yl)-3-(1-(4,6-difluoro-1-methyl-1H-benzo[d]imidazol-2-yl)-2,2,2-trifluoroethyl)urea (85 mg, 0.20 mmol) and DIEA (0.11 mL, 0.61 mmol) in NMP (3 mL) was added azetidin-3-ol (74 mg, 1.0 mmol) at 25° C. under a nitrogen atmosphere. The resulting mixture was stirred for 16 hours at 80° C. under nitrogen. The product was purified by preparative HPLC (Column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 24% B to 34% B in 8 min; wavelength: 254/220 nm; RT (min): 9.63) to afford 1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea (50 mg, 0.11 mmol) as a white solid. LCMS RT 1.132 min, [M+H]⁺ 458.10, LCMS method F.

1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea (50 mg) was further purified by preparative chiral HPLC (column: CHIRAL ART Cellulose-SZ, 2.0*25 cm, 5 μm; mobile phase A: hexane (0.5% 2 M NH₃ in MeOH), mobile phase B: EtOH; flow rate: 20 mL/min; gradient: 50% B isocratic; wavelength: 196/200 nm; RT1 (min): 4.5; RT2 (min): 6.6; sample solvent: MeOH:DCM 1:2; injection volume: 0.5 mL) to give (R)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea and (S)-1-(1-(4,6-difluoro-1-methyl-1H-benzo[d]imidazol-2-yl)-2,2,2-trifluoroethyl)-3-(2-(3-hydroxyazetidin-1-yl)pyrimidin-5-yl)urea, both as a white solid.

Isomer 1: 7 mg, LCMS RT 1.167 min, [M+H]⁺ 458.15, LCMS method F. ¹H NMR (300 MHz, DMSO-d6) δ 8.58 (s, 1H), 8.37 (s, 2H), 7.74 (d, J=9.1 Hz, 1H), 7.50 (dd, J=8.9, 2.3 Hz, 1H), 7.19 (td, J=10.6, 2.2 Hz, 1H), 6.28-6.18 (m, 1H), 5.65 (d, J=6.5 Hz, 1H), 4.59-4.49 (m, 1H), 4.18 (dd, J=9.1, 6.6 Hz, 2H), 3.90 (s, 3H), 3.80-3.71 (m, 2H).

Isomer 2: 5 mg, LCMS RT 1.180 min, [M+H]⁺ 458.10, LCMS method F. ¹H NMR (300 MHz, DMSO-d6) δ 8.58 (s, 1H), 8.37 (s, 2H), 7.74 (d, J=9.1 Hz, 1H), 7.50 (dd, J=8.7, 2.2 Hz, 1H), 7.19 (td, J=10.6, 2.2 Hz, 1H), 6.27-6.15 (m, 1H), 5.65 (d, J=6.5 Hz, 1H), 4.60-4.45 (m, 1H), 4.18 (dd, J=9.1, 6.6 Hz, 2H), 3.90 (s, 3H), 3.73 (dd, J=9.1, 4.6 Hz, 2H).

Additional compounds prepared according to the methods of Example 51 are listed in Table 13 below. Corresponding ¹H NMR and mass spectrometry characterization for these compounds are described in Table 1. Certain compounds in Table 13 below were prepared with other compounds whose preparation is described further below in the Examples.

TABLE 13
Additional exemplary compounds
I-3676

### Example 52

### (R)-1-(2-(azetidin-1-yl)pyrimidin-5-yl)-3-(1-(3-chloro-2,6-difluorophenyl)-2,2,2-trifluoroethyl)urea

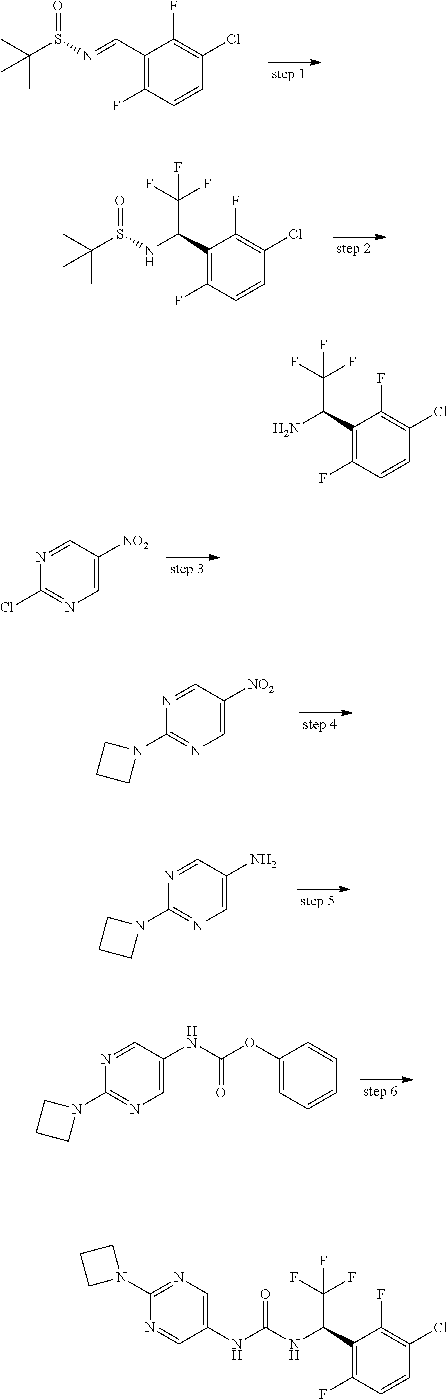

### Step 1. Synthesis of (S)—N—((R)-1-(3-chloro-2,6-difluorophenyl)-2,2,2-trifluoroethyl)-2-methylpropane-2-sulfinamide

A solution of (S,E)-N-(3-chloro-2,6-difluorobenzylidene)-2-methylpropane-2-sulfinamide (1.96 g, 7 mmol) and tetrabutylammoniumdifluorotriphenylsilicate (4.86 g, 9 mmol) in THF (15 mL) was stirred for 1 hour at −60° C. under a nitrogen atmosphere. Trifluoromethyltrimethylsilane (1.14 g, 8 mmol) was added at −60° C. The resulting mixture was stirred at −60° C. for 1 hour. After warming to room temperature water was added, and the solution was extracted with EtOAc (3×100 mL). The combined organic layers were washed with brine (3×50 mL) and dried over anhydrous Na₂SO₄. After filtration, the filtrate was concentrated under reduced pressure. The residue was purified by reverse phase flash chromatography (column, C18 gel; mobile phase, acetonitrile in water (0.1% NH₄OH), gradient: 10% to 90% acetonitrile in 40 min; detector: UV 254 nm) to give (S)—N—((R)-1-(3-chloro-2,6-difluorophenyl)-2,2,2-trifluoroethyl)-2-methylpropane-2-sulfinamide (600 mg, 1.6 mmol) as a white solid. LCMS RT 1.390 min, [M+H]⁺ 350, LCMS method E.

### Step 2. Synthesis of (R)-1-(3-chloro-2,6-difluorophenyl)-2,2,2-trifluoroethan-1-amine

To a stirred solution of (S)—N—((R)-1-(3-chloro-2,6-difluorophenyl)-2,2,2-trifluoroethyl)-2-methylpropane-2-sulfinamide (600 mg, 1.72 mmol) in 1,4-dioxane (10 mL) was added HCl (8.58 mL, 2 M in MeOH, 17.2 mmol) dropwise at room temperature under a nitrogen atmosphere. The resulting mixture was stirred for 2 hours at 30° C. under nitrogen. The solution was concentrated under reduced pressure. The residue was purified by trituration with Et₂O (3×5 mL). The crude product (R)-1-(3-chloro-2,6-difluorophenyl)-2,2,2-trifluoroethan-1-amine (400 mg, 1.5 mmol) was used in the next step directly without further purification. LCMS RT 1.390 min, [M+H]⁺ 246, LCMS method E.

### Step 3. Synthesis of 2-(azetidin-1-yl)-5-nitropyrimidine

To a stirred solution of 2-chloro-5-nitropyrimidine (0.96 g, 6 mmol) and azetidine (0.46 g, 8 mmol) in DMF (5 mL) was added K₂CO₃ (2.76 g, 0.02 mol) at room temperature under a nitrogen atmosphere. The resulting mixture was stirred for 1 hour at 90° C. under nitrogen. The mixture was allowed to cool down to room temperature and diluted with water. The solution was extracted with EtOAc (3×60 mL). The combined organic layers were washed with brine (5×10 mL) and dried over anhydrous Na₂SO₄. After filtration, the filtrate was concentrated under reduced pressure. The residue was purified by silica gel column chromatography, eluting with petroleum ether/EtOAc (1:1) to afford 2-(azetidin-1-yl)-5-nitropyrimidine (430 mg, 2.39 mmol) as a white solid. LCMS RT 0.360 min, [M+H]⁺ 181, LCMS method E.

### Step 4. Synthesis of 2-(azetidin-1-yl)pyrimidin-5-amine

To a stirred solution of 2-(azetidin-1-yl)-5-nitropyrimidine (430 mg, 2.39 mmol) in THF (8 mL) at room temperature was Pd/C (203 mg) added. The flask was purged with hydrogen and stirred for 12 hours under a hydrogen atmosphere. After filtration, the filtrate was concentrated under reduced pressure to afford 2-(azetidin-1-yl)pyrimidin-5-amine (220 mg, 1.46 mmol) as a white solid. LCMS RT 1.076 min, [M+H]⁺ 150.19. LCMS method E.

### Step 5. Synthesis of phenyl (2-(azetidin-1-yl)pyrimidin-5-yl)carbamate

To a stirred solution of 2-(azetidin-1-yl)pyrimidin-5-amine (60 mg, 0.40 mmol) and phenyl carbonochloridate (63 mg, 0.40 mmol) in DMF (2 mL) was added DIEA (0.21 mL, 1.2 mmol) dropwise at room temperature under a nitrogen atmosphere. The resulting mixture was stirred for 1 hour at 0° C. under nitrogen. The crude product was used in the next step directly without purification. LCMS RT 0.755 min, [M+H]⁺ 271, LCMS method E.

### Step 6. Synthesis of (R)-1-(2-(azetidin-1-yl)pyrimidin-5-yl)-3-(1-(3-chloro-2,6-difluorophenyl)-2,2,2-trifluoroethyl)urea

To a stirred solution of phenyl (2-(azetidin-1-yl)pyrimidin-5-yl)carbamate (50 mg, 0.18 mmol) and (R)-1-(3-chloro-2,6-difluorophenyl)-2,2,2-trifluoroethan-1-amine (45 mg, 0.18 mmol) in DMF (1 mL) was added DIEA (72 mg, 0.55 mmol) dropwise at room temperature under a nitrogen atmosphere. The resulting mixture was stirred for 3 hours at 30° C. under nitrogen. The mixture was allowed to cool down to room temperature. The resulting mixture was purified by reverse phase flash chromatography (Column: XBridge Prep OBD C18 Column, 30*150 mm, 10 μm; mobile phase A: water (10 mM NH₄HCO₃+0.05% NH₄OH), mobile phase B: acetonitrile; flow rate: 60 mL/min; gradient: 34% B to 49% B in 8 min; wavelength: 254/220 nm; RT(min): 9.32) to give (R)-1-(2-(azetidin-1-yl)pyrimidin-5-yl)-3-(1-(3-chloro-2,6-difluorophenyl)-2,2,2-trifluoroethyl)urea (4.5 mg, 11 μmol) as a white solid. LCMS RT 1.435 min, [M+H]⁺ 422.05, LCMS method F. ¹H NMR (300 MHz, DMSO-d6) δ 8.67 (s, 1H), 8.36 (s, 2H), 7.84 (td, J=8.8, 5.6 Hz, 1H), 7.60 (d, J=9.9 Hz, 1H), 7.39 (t, J=9.2 Hz, 1H), 6.09 (p, J=9.0 Hz, 1H), 3.99 (t, J=7.4 Hz, 4H), 2.28 (p, J=7.5 Hz, 2H).

### Example 43

Selected compounds of the present disclosure were tested in an ADP-Glo Biochemical PIK3CA Kinase Assay. Compounds to be assayed were plated in 16 doses of 1:2 serial dilutions (20 nL volume each well) on a 1536-well plate, and the plate warmed to room temperature. PIK3CA enzyme (e.g., H1047R, E542K, E545K, or wild-type) (1 μL of 2 nM solution in Enzyme Assay Buffer (comprising 50 mM HEPES pH 7.4, 50 mM NaCl, 6 mM MgCl₂, 5 mM DTT and 0.03% CHAPS)) was added and shaken for 10 seconds and preincubated for 30 minutes. To the well was added 1 μL of 200 μM ATP and 20 μM of diC8-PIP2 in Substrate Assay Buffer (50 mM HEPES pH7.4, 50 mM NaCl, 5 mM DT T and 0.03% CHAPS) to start the reaction, and the plate was shaken for 10 seconds, then spun briefly at 1500 rpm, and then incubated for 60 minutes at room temperature. The reaction was stopped by adding 2 μL of ADP-Glo reagent (Promega), and spinning briefly at 1500 rpm, and then incubating for 40 minutes. ADP-Glo Detection reagent (Promega) was added and the plate spun briefly at 1500 rpm, then incubated for 30 minutes. The plate was read on an Envision 2105 (Perkin Elmer), and the IC₅₀ values were calculated using Genedata software.

Results of the ADP-Glo Biochemical PIK3CA Kinase Assay using H1047R PIK3CA enzyme are presented in Table 1. Compounds having an IC₅₀ less than or equal to 100 nM are represented as “A”; compounds having an IC₅₀ greater than 100 nM but less than or equal to 500 nM are represented as “B”; compounds having an IC₅₀ greater than 500 nM but less than or equal to 1 μM are represented as “C”; compounds having an IC₅₀ greater than 1 μM but less than or equal to 10 μM are represented as “D”; and compounds having an IC₅₀ greater than 10 μM but less than or equal to 100 μM are represented as “E”.

### Example 44

Selected compounds of the present disclosure were tested in a MCF10A Cell-Based PIK3CA Kinase Assay, namely the CisBio Phospho-AKT (Ser473) HTRF assay, to measure the degree of PIK3CA-mediated AKT phosphorylation. MCF10A cells (immortalized non-transformed breast cell line) overexpressing hotspot PIK3CA mutations (including H1047R, E542K, and E545K mutations) were used. Cells were seeded at 5,000 cells per well in DMEM/F12 (Thermo Fisher Scientific) supplemented with 0.5 mg/mL hydrocortisone, 100 ng/mL Cholera Toxin, 10 μg/mL insulin, and 0.5% horse serum. Once plated, cells were placed in a 5% CO₂, 37° C. incubator to adhere overnight.

The following day, compounds were added to the cell plates in 12 doses of 1:3 serial dilutions. The dose response curves were run in duplicate. Compound addition was carried out utilizing an Echo 55 Liquid Handler acoustic dispenser (Labcyte). The cell plates were incubated for 2 hours in a 5% CO₂, 37° C. incubator. Following compound incubation, the cells were lysed for 60 min at room temperature. Finally, a 4-hour incubation with the HTRF antibodies was performed at room temperature. All reagents, both lysis buffer and antibodies, were used from the CisBio pAKT 5473 HTRF assay kit, as per the manufacturers protocol. Plates were read on an Envision 2105 (Perkin Elmer), and the IC₅₀ values were calculated using Genedata software.

Results of the MCF10A Cell-Based PIK3CA Kinase Assay are presented in Table 1. Compounds having an IC₅₀ less than or equal to 1 μM are represented as “A”; compounds having an IC₅₀ greater than 1 μM but less than or equal to 5 μM are represented as “B”; compounds having an IC₅₀ greater than 5 μM but less than or equal to 10 μM are represented as “C”; compounds having an IC₅₀ greater than 10 μM but less than or equal to 36 μM are represented as “D”; and compounds having an IC₅₀ greater than 36 μM but less than or equal to 100 μM are represented as “E”.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

LENGTHY TABLES
The patent application contains a lengthy table section. A copy of the table is available in electronic form from the USPTO web site (https://seqdata.uspto.gov/docdetail?docId=US20260035335A1). An electronic copy of the table will also be available from the USPTO upon request and payment of the fee set forth in 37 CFR 1.19(b)(3).

1-46. (canceled)

47. A compound, which is: 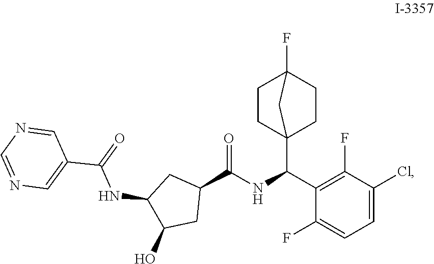 [SMILES: O=C(N[C@H]1C[C@@H](C(=O)N[C@H](c2c(F)ccc(Cl)c2F)C23CCC(F)(CC2)C3)C[C@H]1O)c1cncnc1]
or a pharmaceutically acceptable salt thereof.

48. A compound, which is: 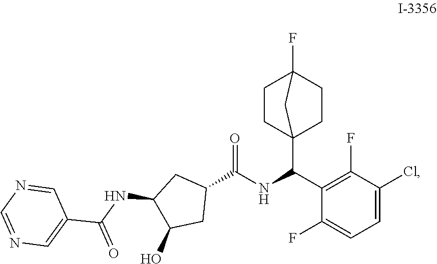
or a pharmaceutically acceptable salt thereof.

49. A pharmaceutical composition comprising the compound of claim 47, or a pharmaceutically acceptable salt thereof, and a pharmaceutically acceptable carrier, adjuvant, or vehicle.

50. A pharmaceutical composition comprising the compound of claim 48, or a pharmaceutically acceptable salt thereof, and a pharmaceutically acceptable carrier, adjuvant, or vehicle.

51. A method of inhibiting PI3Kα signaling activity in a subject in need thereof, comprising administering to the subject a therapeutically effective amount of the compound of claim 47, or a pharmaceutically acceptable salt thereof, or a pharmaceutical composition thereof.

52. A method of inhibiting PI3Kα signaling activity in a subject in need thereof, comprising administering to the subject a therapeutically effective amount of the compound of claim 48, or a pharmaceutically acceptable salt thereof, or a pharmaceutical composition thereof.

53. A method of treating a PI3Kα-mediated disorder in a subject, comprising administering to the subject a therapeutically effective amount of the compound of claim 47, or a pharmaceutically acceptable salt thereof, or a pharmaceutical composition thereof.

54. A method of treating a PI3Kα-mediated disorder in a subject, comprising administering to the subject a therapeutically effective amount of the compound of claim 48, or a pharmaceutically acceptable salt thereof, or a pharmaceutical composition thereof.

55. A method of treating a cellular proliferative disease in a subject, comprising administering to the subject a therapeutically effective amount of the compound of claim 47, or a pharmaceutically acceptable salt thereof, or a pharmaceutical composition thereof.

56. A method of treating a cellular proliferative disease in a subject, comprising administering to the subject a therapeutically effective amount of the compound of claim 48, or a pharmaceutically acceptable salt thereof, or a pharmaceutical composition thereof.

57. The method of claim 55, wherein the cellular proliferative disease is cancer.

58. The method of claim 57, wherein the cancer is breast cancer.

59. The method of claim 57, wherein the cancer is ovarian cancer.

60. The method of claim 59, wherein the ovarian cancer is clear cell ovarian cancer.

61. The method of claim 55, wherein the subject has PI3Kα containing at least one of the following mutations: H1047R, E542K, and E545K.

62. The method of claim 56, wherein the cellular proliferative disease is cancer.

63. The method of claim 62, wherein the cancer is breast cancer.

64. The method of claim 62, wherein the cancer is ovarian cancer.

65. The method of claim 64, wherein the ovarian cancer is clear cell ovarian cancer.

66. The method of claim 56, wherein the subject has PI3Kα containing at least one of the following mutations: H1047R, E542K, and E545K.
